(19) United States
(12) Patent Application Publication
(10) Pub. No.: US 2018/0318438 A1
(43) Pub. Date: Nov. 8, 2018

(54) ANTIBODY DRUG CONJUGATES OF KINESIN SPINDEL PROTEIN (KSP) INHIBITORS WITH ANTI-TWEAKR-ANTIBODIES

(71) Applicant: Bayer Pharma Aktiengesellschaft, Berlin (DE)

(72) Inventors: Hans-Georg LERCHEN, Leverkusen (DE); Anne-Sophie REBSTOCK, Champagne au Mont d'Or (FR); Yolanda CANCHO GRANDE, Leverkusen (DE); Sven WITTROCK, Berlin (DE); Beatrix STELTE-LUDWIG, Wülfrath (DE); Stephan MÄRSCH, Köln (DE); Uwe GRITZAN, Köln (DE); Christoph MAHLERT, Wuppertal (DE); Simone GREVEN, Dormagen (DE); Anette SOMMER, Berlin (DE); Sandra BERNDT, Hohen-Neuendorf (DE); Lisa DIETZ, Wuppertal (DE)

(73) Assignee: Bayer Pharma Aktiengesellschaft, Berlin (DE)

(21) Appl. No.: 15/739,136

(22) PCT Filed: Jun. 20, 2016
(86) PCT No.: PCT/EP2016/064128

§ 371 Date: Dec. 21, 2017

(30) Foreign Application Priority Data
Jun. 23, 2015 (EP) 15173477.9

(51) Int. Cl. A61K 47/68; C07K 16/28; A61K 31/40; A61K 31/4025; A61P 35/00

(52) U.S. Cl. CPC A61K 47/6803; C07K 16/2878; A61K 47/6849; A61K 31/40; A61K 31/4025; A61K 2039/572; C07K 2317/565; C07K 2317/56; C07K 2317/75; C07K 2317/76; C07K 2317/73; A61P 35/00

(57) ABSTRACT

The present application relates to novel binder drug conjugates (ADCs), to active metabolites of these ADCs, to processes for preparing these ADCs, to the use of these ADCs for the treatment and/or prophylaxis of diseases and to the use of these ADCs for preparing medicaments for treatment and/or prophylaxis of diseases, in particular hyperproliferative and/or angiogenic disorders such as, for example, cancer diseases. Such treatments can be effected as monotherapy or else in combination with other medicaments or further therapeutic measures.

### INTRODUCTION AND STATE OF THE ART

The invention relates to binder drug conjugates (ADCs) of kinesin spindle protein inhibitors, to active metabolites of these ADCs, to processes for preparing these ADCs, to the use of these ADCs for the treatment and/or prophylaxis of diseases and to the use of these ADCs for preparing medicaments for treatment and/or prevention of diseases, in particular hyperproliferative and/or angiogenic disorders such as, for example, cancer diseases. Such treatments can be effected as monotherapy or else in combination with other medicaments or further therapeutic measures.

Cancers are the consequence of uncontrolled cell growth of the most diverse tissues. In many cases the new cells penetrate into existing tissue (invasive growth), or they metastasize into remote organs. Cancers occur in a wide variety of different organs and often have tissue-specific courses. The term “cancer” as a generic term therefore describes a large group of defined diseases of different organs, tissue and cell types.

Some tumours at early stages can be removed by surgical and radiotherapy measures. Metastased tumours as a rule can only be treated palliatively by chemotherapeutics. The aim here is to achieve the optimum combination of an improvement in the quality of life and prolonging of life.

Conjugates of binder proteins with one or more active compound molecules are known, in particular in the form of antibody drug conjugates (ADCs) in which an internalising antibody directed against a tumour-associated antigen is covalently attached via a linker to a cytotoxic agent. Following introduction of the ADCs into the tumour cell and subsequent dissociation of the conjugate, either the cytotoxic agent itself or a cytotoxic metabolite formed therefrom is released within the tumour cell and can unfold its action therein directly and selectively. In this manner, in contrast to conventional chemotherapy, damage to normal tissue is contained in significantly narrower limits [see, for example, J. M. Lambert, Curr. Opin. Pharmacol. 5, 543-549 (2005); A. M. Wu and P. D. Senter, Nat. Biotechnol. 23, 1137-1146 (2005); P. D. Senter, Curr. Opin. Chem. Biol. 13, 235-244 (2009); L. Ducry and B. Stump, Bioconjugate Chem. 21, 5-13 (2010)]. Thus, WO2012/171020 describes ADCs in which a plurality of toxophor molecules are attached via a polymeric linker to an antibody. As possible toxophors, WO2012/171020 mentions, among others, the substances SB 743921, SB 715992 (Ispinesib), MK-0371, AZD8477, AZ3146 and ARRY-520.

The substances mentioned last are kinesin spindle protein inhibitors. Kinesin spindle protein (KSP, also known as Eg5, HsEg5, KNSL1 or KIF11) is a kinesin-like motorprotein which is essential for the bipolar mitotic spindle to function. Inhibition of KSP leads to mitotic arrest and, over a relatively long term, to apoptosis (Tao et al., Cancer Cell 2005 Jul. 8(1), 39-59). After the discovery of the first cell-permeable KSP inhibitor, monastrol, KSP inhibitors have established themselves as a class of novel chemotherapeutics (Mayer et al., Science 286: 971-974, 1999) and have been the subject of a number of patent applications (e.g. WO2006/044825; WO2006/002236; WO2005/051922; WO2006/060737; WO03/060064; WO03/040979; and WO03/049527). However, since KSP is active only during a relatively short period of time during the mitosis phase, KSP inhibitors have to be present in a sufficiently high concentration during this phase. WO2014/151030 discloses ADCs including certain KSP inhibitors.

### SUMMARY OF THE INVENTION

Against this background it is an object of the present invention to provide substances which, after administration at a relatively low concentration, unfold apoptotic action and may therefore be of benefit for cancer therapy.

To achieve this object, the invention provides conjugates of a moderately agonistically or non-agonistically acting anti-TWEAKR antibody, such as the ITEM-4 antibody originating from the mouse, and chimeric or humanized variants of this antibody, with compounds of the formula (I) below, where one or more of the compounds of the formula (I) are attached to the antibody via a linker L.

The invention shows that a significantly improved tumour selectivity is to be observed with conjugates using an anti-TWEAKR antibody with reduced agonistic action.

ITEM-4 is an anti-TWEAKR antibody described by Nakayama et al. (Nakayama, et al., 2003, Biochem Biophy Res Comm, 306:819-825). Humanized variants of this antibody based on CDR grafting are described by Zhou et al. (Zhou et al., 2013, J Invest Dermatol. 133(4):1052-62) and in WO 2009/020933.

The antibody is preferably a humanized or chimeric monoclonal anti-TWEAKR antibody. Particular preference is given to the humanized or chimeric ITEM-4 variants: TPP-7005, TPP-7006, TPP-7007, TPP-7053, TPP-7073, TPP-7074, TPP-7075 and TPP-7076.

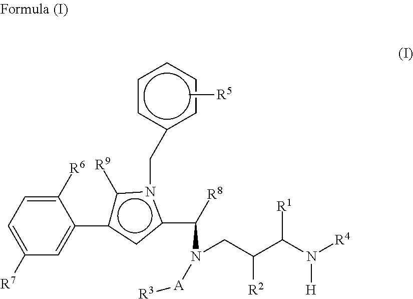

where
R¹ represents H, -L-#1, -MOD or —(CH₂)₀₋₃Z, where Z represents —H, —NHY³, —OY³, —SY³, halogen, —CO—NY¹Y² or —CO—OY³,
where Y¹ and Y² independently of one another represent H, NH₂, —(CH₂CH₂O)₀₋₃—(CH₂)₀₋₃Z′ (e.g. —(CH₂)₀₋₃Z′) or —CH(CH₂W)Z′, and Y³ represents H or —(CH₂)₀₋₃Z′, where Z′ represents H, NH₂, SO₃H, COOH, —NH—CO—CH₂—CH₂—CH(NH₂)COOH or —(CO—NH—CHY⁴)₁₋₃COOH, where W represents H or OH,
where Y⁴ represents straight-chain or branched C₁₋₆ alkyl which is optionally substituted by —NHCONH₂, or represents aryl or benzyl which are optionally substituted by —NH₂;
R² represents H, -MOD, —CO—CHY⁴—NHY⁵ or —(CH₂)₀₋₃Z,
where Z represents —H, halogen, —OY³, —SY³, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where Y⁴ represents straight-chain or branched C₁₋₆-alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl;
R⁴ represents H, -L-#1, -SG_{lys}-(CO)₀₋₁—R^{4′}, —CO—CHY⁴—NHY⁵ or —(CH₂)₀₋₃Z,
where SG_{lys} represents a group which can be cleaved by a lysosomal enzyme, in particular a group consisting of a di- or tripeptide, R^{4′} represents a C₁₋₁₀-alkyl, C₅₋₁₀-aryl or C₆₋₁₀-aralkyl, C₅₋₁₀-heteroalkyl, C₁₋₁₀-alkyl-O—C₆₋₁₀-aryl, C₅₋₁₀-heterocycloalkyl, heteroaryl, heteroarylalkyl, heteroarylalkoxy, C₁₋₁₀-alkoxy, C₆₋₁₀-aryloxy or C₆₋₁₀-aralkoxy, C₅₋₁₀-heteroaralkoxy, C₁₋₁₀-alkyl-O—C₆₋₁₀-aryloxy, C₅₋₁₀-heterocycloalkoxy group which may be mono- or polysubstituted by —NH₂, —NH-alkyl, —N(alkyl)₂, NH—CO-alkyl, N(alkyl)-COalkyl, —SO₃H, —SO₂NH₂, —SO₂—N(alkyl)₂, —COOH, —CONH₂, —CON(alkyl)₂ or —OH, —H or a group —Oₓ—(CH₂CH₂O)_{y}—R^{4″} (where x represents 0 or 1 and v represents a number from 1 to 10 and R^{4″} represents —H, -alkyl (preferably C₁₋₁₂-alkyl), —CH₂—COOH, —CH₂—CH₂—COOH or —CH₂—CH₂—NH₂), where after cleavage a primary amine group is present (corresponds to R⁴=H);
where Z represents —H, halogen, —OY³, —SY³, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where Y⁴ represents straight-chain or branched C₁₋₆-alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl;
or R² and R⁴ together (with formation of a pyrrolidine ring) represent —CH₂—CHR¹¹— or —CHR¹¹—CH₂—, where R¹¹ represents —H, —NH₂, —SO₃H, —COOH, —SH, halogen (in particular F or Cl), C₁₋₄-alkyl, C₁₋₄-haloalkyl, C₁₋₄-alkoxy, hydroxyl-substituted C₁₋₄-alkyl, COO(C₁₋₄-alkyl) or OH;
A represents CO, SO, SO₂, SO₂NH or CNNH₂;
R³ represents -L-#1, -MOD or an optionally substituted alkyl, cycloalkyl, aryl, heteroaryl, heteroalkyl, heterocycloalkyl group, preferably -L-#1 or a C₁₋₁₀-alkyl, C₆₋₁₀-aryl or C₆₋₁₀-aralkyl, C₅₋₁₀-heteroalkyl, C₁₋₁₀-alkyl-O—C₆₋₁₀-aryl or C₅₋₁₀-heterocycloalkyl group which may be substituted by 1-3 —OH groups, 1-3 halogen atoms, 1-3 halogenated alkyl groups (each having 1-3 halogen atoms), 1-3 O-alkyl groups, 1-3 —SH groups, 1-3 —S-alkyl groups, 1-3 —O—CO-alkyl groups, 1-3 —O—CO—NH-alkyl groups, 1-3 —NH—CO-alkyl groups, 1-3 —NH—CO—NH-alkyl groups, 1-3 —S(O)ₙ-alkyl groups, 1-3 —SO₂—NH-alkyl groups, 1-3 —NH-alkyl groups, 1-3 —N(alkyl)₂ groups, 1-3 —NH₂ groups or 1-3 —(CH₂)₀₋₃Z groups, where n represents 0, 1 or 2, Z represents —H, halogen, —OY³, —SY³, —NHY³, —CO—NY¹Y² or —CO—OY³, where Y¹ and Y² independently of one another represent H, NH₂ or —(CH₂)₀₋₃Z′ and Y³ represents H, —(CH₂)₀₋₃—CH(NHCOCH₃)Z′, —(CH₂)₀₋₃—CH(NH₂)Z′ or —(CH₂)₀₋₃Z′, where Z′ represents H, SO₃H, NH₂ or COOH (where “alkyl” preferably represents C₁₋₁₀-alkyl);
R⁵ represents H, NH₂, NO₂, halogen (in particular F, Cl, Br), —CN, CF₃, —OCF₃, —CH₂F, —CH₂F, SH or —(CH₂)₀₋₃Z, where Z represents —H, —OY³, —SY³, halogen, NHY³, —CO—NY¹Y² or —CO—OY³, where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
R⁶ and R⁷ independently of one another represent H, cyano, (optionally fluorinated) C₁₋₁₀-alkyl, (optionally fluorinated) C₂₋₁₀-alkenyl, (optionally fluorinated) C₂₋₁₀-alkynyl, hydroxy, NO₂, NH₂, COOH or halogen (in particular F, Cl, Br),
R⁸ represents (optionally fluorinated) C₁₋₁₀-alkyl, (optionally fluorinated) C₂₋₁₀-alkenyl, (optionally fluorinated) C₂₋₁₀-alkynyl, (optionally fluorinated) C₄₋₁₀-cycloalkyl or —(CH₂)₀₋₂—(HZ²), where HZ² represents a 4- to 7-membered heterocycle having up to two heteroatoms selected from the group consisting of N, O and S, where each of these groups may be substituted by —OH, CO₂H or NH₂;
R⁹ represents H, F, CH₃, CF₃, CH₂F or CHF₂;
where one of the substituents R¹, R³ or R⁴ represents or (in the case of R⁸) contains -L-#1,
L represents the linker and #1 represents the bond to the binder or derivative thereof,
where -MOD represents —(NR¹⁰)ₙ-(G1)ₒ-G2-G3, where
R¹⁰ represents H or C₁-C₃-alkyl;
G1 represents —NHCO— or —CONH— (where, if G1 represents —NHCO—, R¹⁰ does not represent NH₂);
n represents 0 or 1;
o represents 0 or 1; and
G2 represents a straight-chain and/or branched hydrocarbon group which has 1 to 10 carbon atoms and which may be interrupted once or more than once by one or more of the groups —O—, —S—, —SO—, SO₂, -NRy-, -NRyCO—, CONRy-, -NRyNRy-, —SO₂NRyNRy-, -CONRyNRy- (where R^{y} represents H, phenyl, C1-C10-alkyl, C2-C10-alkenyl or C2-C10-alkynyl, each of which may be substituted by NHCONH₂, —COOH, —OH, —NH₂, NH—CNNH₂, sulphonamide, sulphone, sulphoxide or sulphonic acid), —CO—, or —CR^{x}═N—O— (where Rx represents H, C1-C3-alkyl or phenyl), where the hydrocarbon chain including any side chains may be substituted by —NHCONH₂, —COOH, —OH, —NH₂, NH—CNNH₂, sulphonamide, sulphone, sulphoxide or sulphonic acid, G3 represents —H or —COOH, and where the group -MOD preferably has at least one group —COOH;
and the salts, solvates, salts of the solvates and epimers thereof.

The conjugates according to the invention can have chemically labile linkers, enzymatically labile linkers or stable linkers. Particular preference is given to stable linkers and linkers which can be cleaved by a protease.

The invention furthermore provides processes for preparing the conjugates according to the invention, and also precursors and intermediates for the preparation.

The preparation of the conjugates according to the invention regularly comprises the following steps:

preparation of a linker precursor which optionally carries protective groups and has a reactive group which is capable of coupling to the antibody;
conjugation of the linker precursor to the derivative, which optionally carries protective groups, of a KSP inhibitor of the formula (I), where in these formulae there is as yet no bond to a linker, giving a reactive KSP inhibitor/linker conjugate which optionally carries protective groups;
removal of any protective groups present in the KSP inhibitor/linker conjugate and
conjugation of the antibody to the KSP inhibitor/linker conjugate, giving the antibody/KSP inhibitor conjugate according to the invention.

Attachment of the reactive group may also take place after the construction of an optionally protected KSP inhibitor/linker precursor conjugate.

Depending on the linker, succinimide-linked ADCs may, after conjugation, be converted according to Scheme 26 into the open-chain succinamides, which have an advantageous stability profile.

As illustrated above, conjugation of the linker precursor to a low-molecular-weight KSP inhibitor can be by substitution of a hydrogen atom at R¹, R³ or R⁴ in formula (I) by the linker. In the synthesis steps prior to the conjugation, any functional groups present may also be present in protected form. Prior to the conjugation step, these protective groups are removed by known methods of peptide chemistry. The conjugation can take place chemically by various routes, as shown in an exemplary manner in Schemes 20 to 31 in the examples. In particular, it is optionally possible to modify the low-molecular weight KSP inhibitor for conjugation to the linker, for example by introduction of protective groups or leaving groups to facilitate substitution.

In particular, the invention provides novel low-molecular-weight KSP inhibitors conjugated to a moderately agonistically or non-agonistically acting anti-TWEAKR antibody such as e.g. ITEM-4. These KSP inhibitors or their antibody conjugates have the following general formula (II):

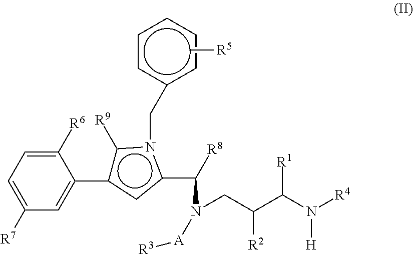

where
R¹ represents H, -L-BINDER, -MOD or —(CH₂)₀₋₃Z, where Z represents —H, —NHY³, —OY³, —SY³, halogen, —CO—NY¹Y² or —CO—OY³,
   where
   Y¹ and Y² independently of one another represent H, NH₂, —(CH₂CH₂O)₀₋₃ —(CH₂)₀₋₃Z′ (e.g. —(CH₂)₀₋₃Z′) or —CH(CH₂W)Z′,
   Y³ represents H or —(CH₂)₀₋₃Z′,
   Z′ represents H, NH₂, SO₃H, COOH, —NH—CO—CH₂—CH₂—CH(NH₂)COOH or —(CO—NH—CHY⁴)₁₋₃COOH;
   W represents H or OH,
   Y⁴ represents straight-chain or branched C₁₋₆ alkyl which is optionally substituted by —NH—C(═O)—NH₂, or represents aryl or benzyl which are optionally substituted by —NH₂;
R² represents H, -MOD, —C(═O)—CHY⁴—NHY⁵ or —(CH₂)₀₋₃Z,
   or
R² and R⁴ together (with formation of a pyrrolidine ring) represent —CH₂—CHR¹¹— or —CHR¹¹—CH₂—,
   where
   R¹¹ represents —H, —NH₂, —SO₃H, —COOH, —SH, halogen (in particular F or Cl), C₁₋₄-alkyl, C₁₋₄-haloalkyl, C₁₋₄-alkoxy, hydroxyl-substituted C₁₋₄-alkyl, COO(C₁₋₄-alkyl) or OH;
   Z represents —H, halogen, —OY³, —SY³, NHY³, —CO—NY¹Y² or —CO—OY³,
      Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH; where Y⁴ represents straight-chain or branched C₁₋₆-alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl;
R⁴ represents H, -L-BINDER, -SG_{lys}-(CO)₀₋₁—R^{4′}, —CO—CHY⁴—NHY⁵ or —(CH₂)₀₋₃Z,
where SG_{lys} represents a group which can be cleaved by a lysosomal enzyme, in particular a group consisting of a di- or tripeptide, R^{4′} represents a C₁₋₁₀-alkyl, C₅₋₁₀-aryl or C₆₋₁₀-aralkyl, C₅₋₁₀-heteroalkyl, C₁₋₁₀-alkyl-O—C₆₋₁₀-aryl, C₅₋₁₀-heterocycloalkyl, heteroaryl, heteroarylalkyl, heteroarylalkoxy, C₁₋₁₀-alkoxy, C₆₋₁₀-aryloxy or C₆₋₁₀-aralkoxy, C₅₋₁₀-heteroaralkoxy, C₁₋₁₀-alkyl-O—C₆₋₁₀-aryloxy, C₅₋₁₀-heterocycloalkoxy group which may be mono- or polysubstituted by —NH₂, —NH-alkyl, —N(alkyl)₂, NH—CO-alkyl, N(alkyl)-COalkyl, —SO₃H, —SO₂NH₂, —SO₂—N(alkyl)₂, —COOH, —CONH₂, —CON(alkyl)₂ or —OH, —H or a group —Oₓ—(CH₂CH₂O)_{y}—R^{4″} (where x represents 0 or 1 and v represents a number from 1 to 10 and R^{4″} represents —H, -alkyl (preferably C₁₋₁₂-alkyl), —CH₂—COOH, —CH₂—CH₂—COOH or —CH₂—CH₂—NH₂), where after cleavage a primary amine group is present (corresponds to R⁴=H);
where Z represents —H, halogen, —OY³, —SY³, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where Y⁴ represents straight-chain or branched C₁₋₆-alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl;
or R² and R⁴ together (with formation of a pyrrolidine ring) represent —CH₂—CHR¹¹— or —CHR¹¹—CH₂—, where R¹¹ represents H, NH₂, SO₃H, COOH, SH, halogen (in particular F or Cl), C₁₋₄-alkyl, C₁₋₄-haloalkyl, C₁₋₄-alkoxy, hydroxyl-substituted C₁₋₄-alkyl, COO(C₁₋₄-alkyl) or OH;
A represents —C(═O)—, —S(═O)—, —S(═O)₂—, —S(═O)₂—NH— or —CNNH₂—;
R³ represents -L-BINDER, -MOD or an optionally substituted alkyl, cycloalkyl, aryl, heteroaryl, heteroalkyl, heterocycloalkyl group, preferably -L-BINDER or a C₁₋₁₀-alkyl, C₆₋₁₀-aryl or C₆₋₁₀-aralkyl, C₅₋₁₀-heteroalkyl, C₁₋₁₀-alkyl-O—C₆₋₁₀-aryl or C₅₋₁₀-heterocycloalkyl group which may be substituted by 1-3 —OH groups, 1-3 halogen atoms, 1-3 halogenated alkyl groups (each having 1-3 halogen atoms), 1-3 O-alkyl groups, 1-3 —SH groups, 1-3 —S-alkyl groups, 1-3 —O—CO-alkyl groups, 1-3 —O—CO—NH-alkyl groups, 1-3 —NH—CO-alkyl groups, 1-3 —NH—CO—NH-alkyl groups, 1-3 —S(O)ₙ-alkyl groups, 1-3 —SO₂—NH-alkyl groups, 1-3 —NH-alkyl groups, 1-3 —N(alkyl)₂ groups, 1-3 —NH₂ groups or 1-3 —(CH₂)₀₋₃Z groups, where Z represents —H, halogen, —OY³, —SY³, —NHY³, —CO—NY¹Y² or —CO—OY³, where Y¹ and Y² independently of one another represent H, NH₂ or —(CH₂)₀₋₃Z′ and Y³ represents H, —(CH₂)₀₋₃—CH(NHCOCH₃)Z′, —(CH₂)₀₋₃—CH(NH₂)Z′ or —(CH₂)₀₋₃Z′, where Z′ represents H, SO₃H, NH₂ or COOH
(where “alkyl” preferably represents C₁₋₁₀-alkyl);
n represents 0, 1 or 2,
R⁵ represents H, NH₂, NO₂, halogen (in particular F, Cl, Br), —CN, CF₃, —OCF₃, —CH₂F, —CH₂F, SH or —(CH₂)₀₋₃Z, where Z represents —H, —OY³, —SY³, halogen, NHY³, —CO—NY¹Y² or —CO—OY³, where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
   R⁸ represents (optionally fluorinated) C₁₋₁₀-alkyl, (optionally fluorinated) C₂₋₁₀-alkenyl, (optionally fluorinated) C₂₋₁₀-alkynyl, (optionally fluorinated) C₄₋₁₀-cycloalkyl or —(CH₂)₀₋₂—(HZ²), where HZ² represents a 4- to 7-membered heterocycle having up to two heteroatoms selected from the group consisting of N, O and S (preferably oxetane), where each of these groups may be substituted by —OH, CO₂H or NH₂;
R⁹ represents H, F, CH₃, CF₃, CH₂F or CHF₂;
where L represents a linker and BINDER represents a moderately agonistically or non-agonistically acting anti-TWEAKR antibody, such as ITEM-4 and chimeric or humanized variants of ITEM-4, where the binder may optionally be attached to a plurality of active compound molecules,
where one representative of R¹, R³ and R⁴ represents -L-BINDER;
R⁶ and R⁷ independently of one another represent H, cyano, (optionally fluorinated) C₁₋₁₀-alkyl, (optionally fluorinated) C₂₋₁₀-alkenyl, (optionally fluorinated) C₂₋₁₀-alkynyl, hydroxy, NO₂, NH₂, COOH or halogen (in particular F, Cl, Br),
where -MOD represents —(NR¹⁰)ₙ-(G1)ₒ-G2-G3, where
R¹⁰ represents H or C₁-C₃-alkyl;
G1 represents —NHCO— or —CONH— (where, if G1 represents —NHCO—, R¹⁰ does not represent NH₂);
n represents 0 or 1;
o represents 0 or 1; and
G2 represents a straight-chain and/or branched hydrocarbon group which has 1 to 10 carbon atoms and which may be interrupted once or more than once by one or more of the groups —O—, —S—, —SO—, SO₂, -NRy-, -NRyCO—, CONRy-, -NRyNRy-, —SO₂NRyNRy-, -CONRyNRy- (where R^{y} represents H, phenyl, C1-C10-alkyl, C2-C10-alkenyl or C2-C10-alkynyl, each of which may be substituted by NHCONH₂, —COOH, —OH, —NH₂, NH—CNNH₂, sulphonamide, sulphone, sulphoxide or sulphonic acid), —CO—, —CR^{x}═N—O— (where Rx represents H, C1-C3-alkyl or phenyl), where the hydrocarbon chain including any side chains may be substituted by —NHCONH₂, —COOH, —OH, —NH₂, NH—CNNH₂, sulphonamide, sulphone, sulphoxide or sulphonic acid, G3 represents —H or —COOH, where the group -MOD preferably has at least one group —COOH;
and the salts, solvates, salts of the solvates and epimers thereof.

### DESCRIPTION OF THE FIGURES

FIG. 1: Annotated sequences of the antibodies. For each of the antibodies or antibody fragments, the CDR regions (HCDR1, HCDR2, HCDR3, LCDR1, LCDR2, LCDR3) and the variable regions (VH, VL) are emphasized.

FIG. 2: Sequence protocol.

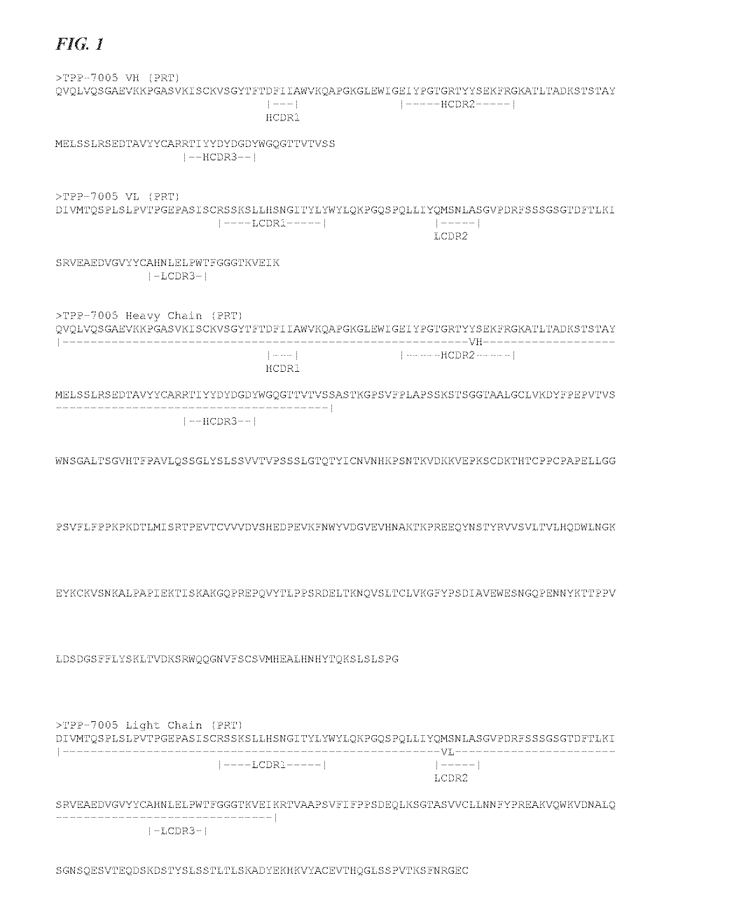

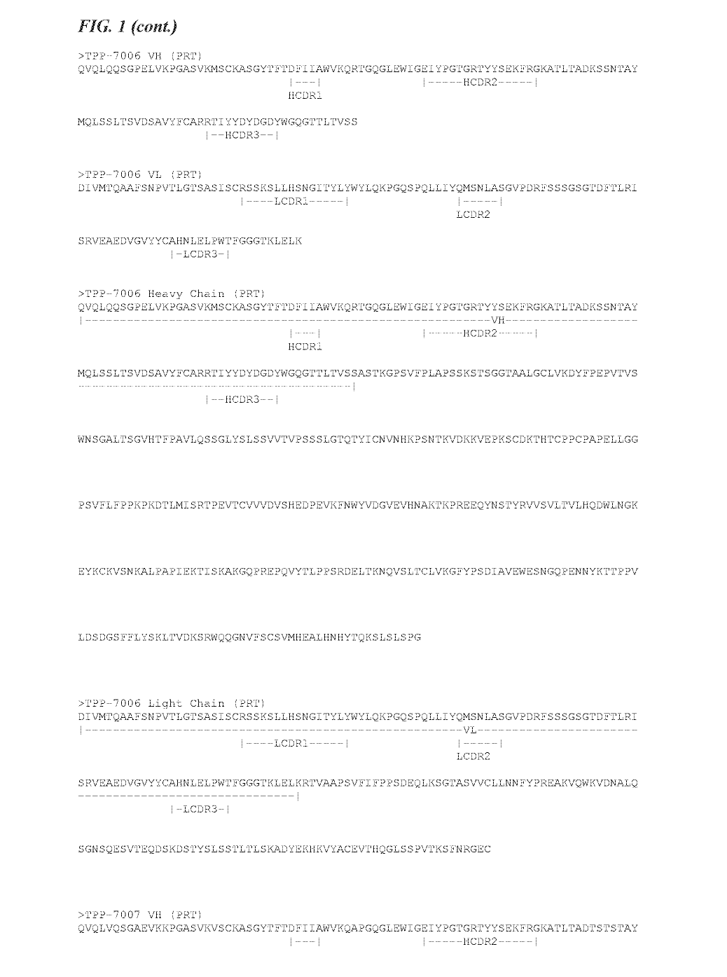

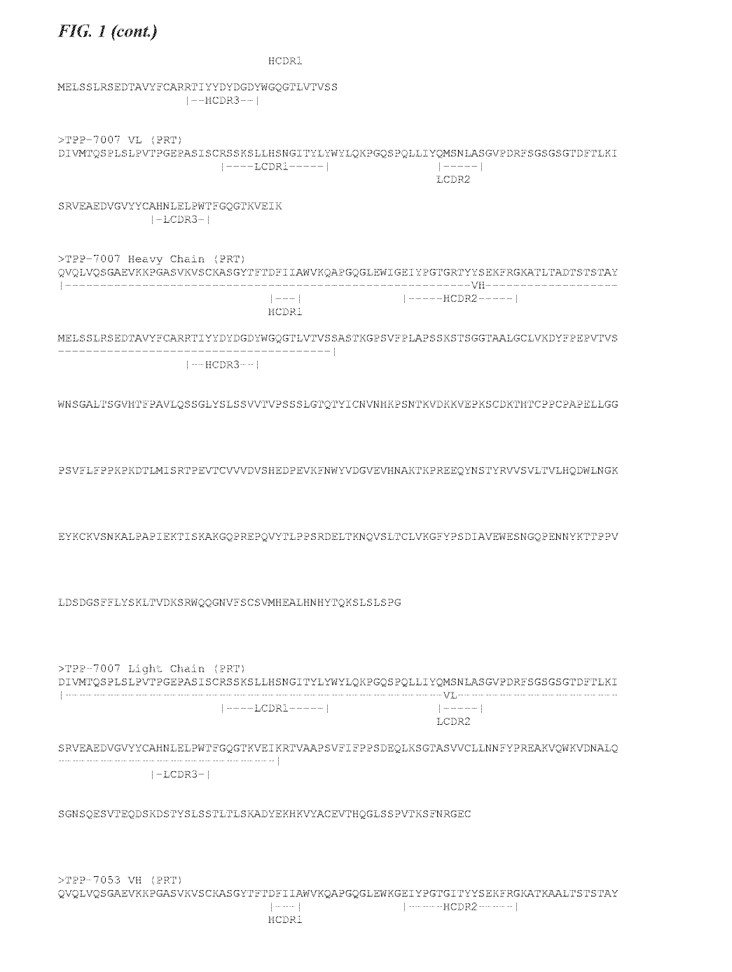

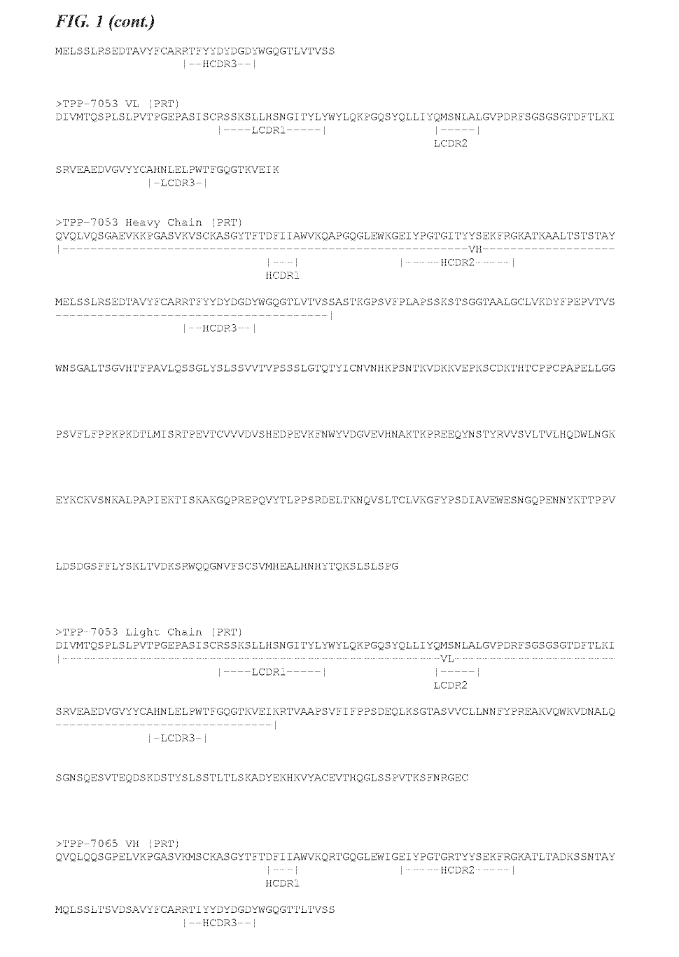

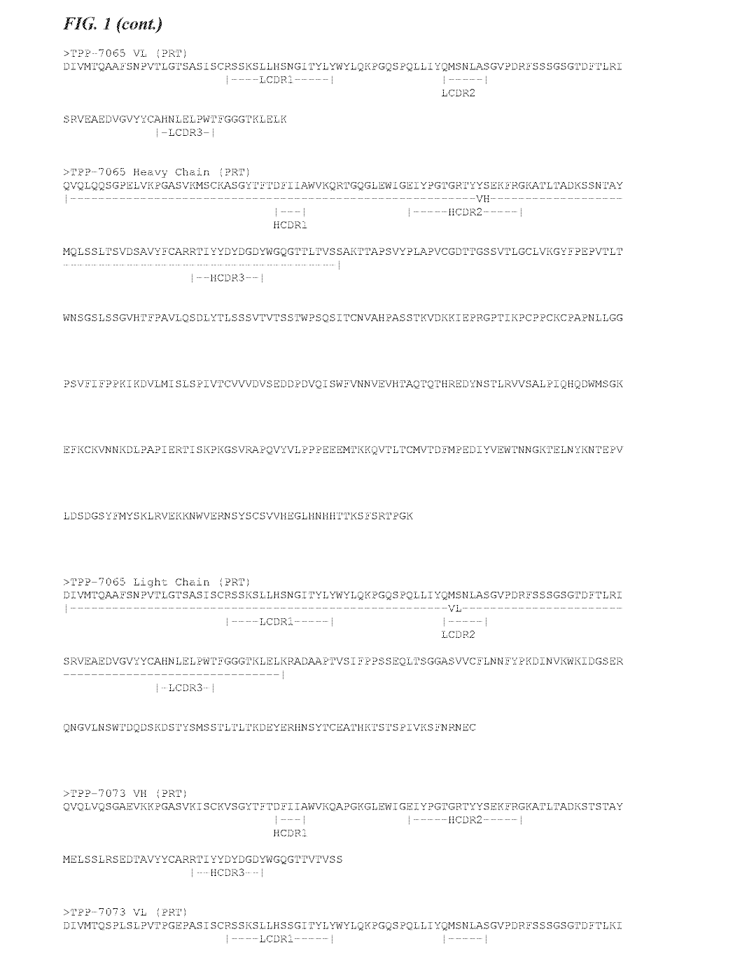

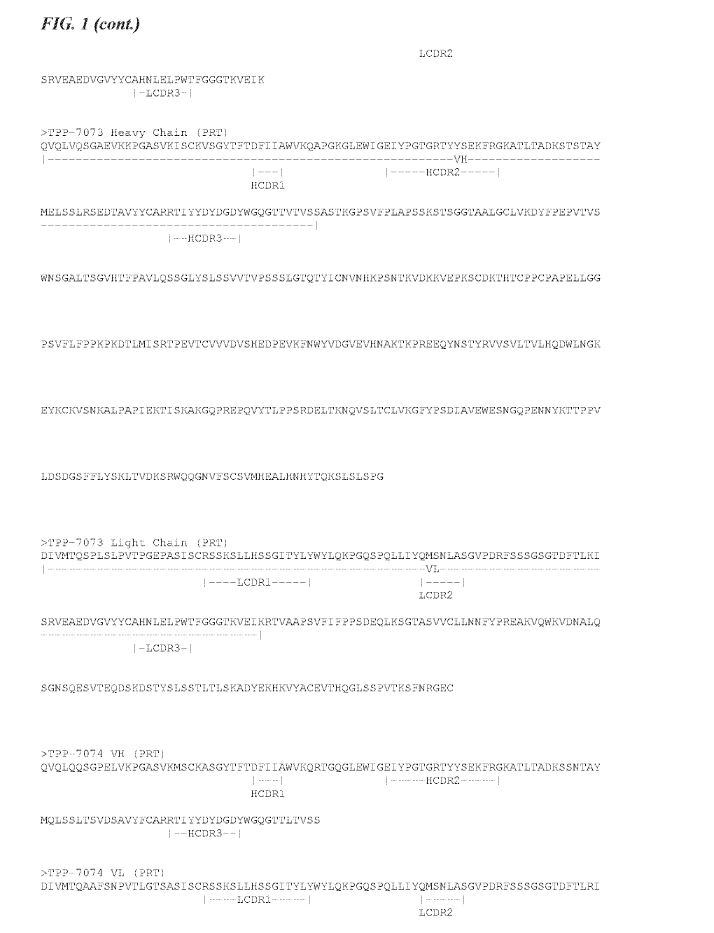

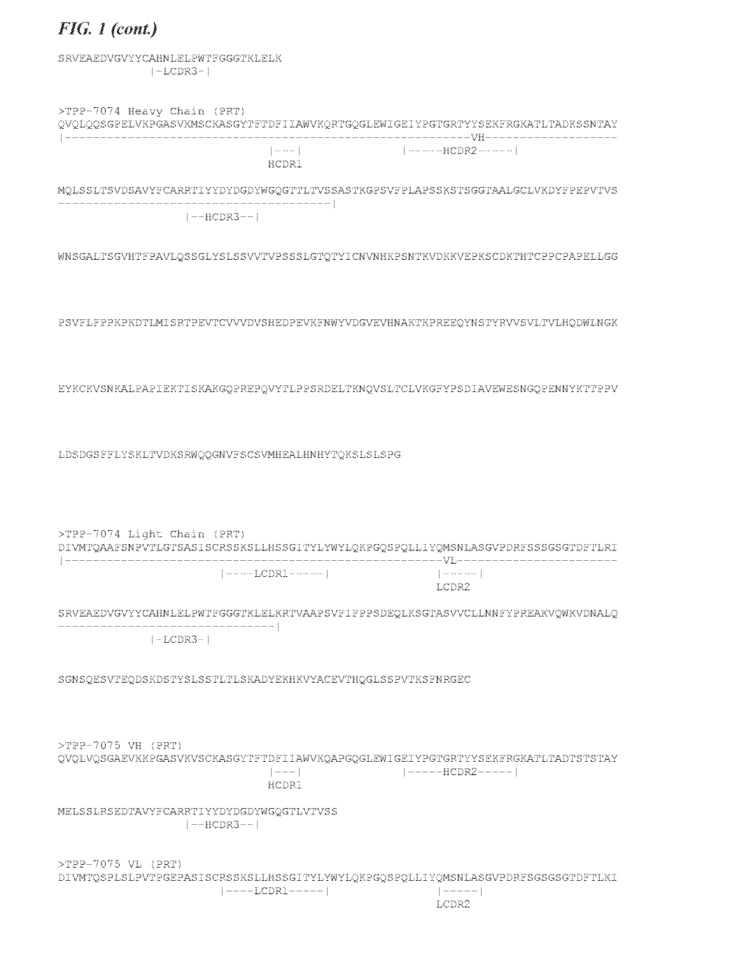

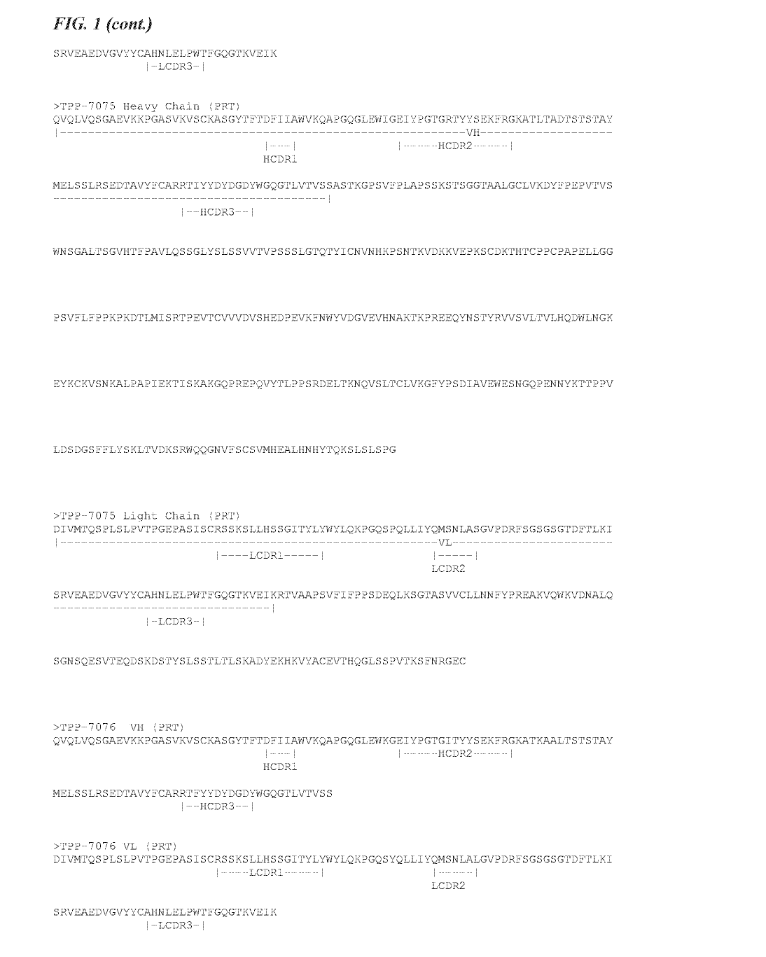

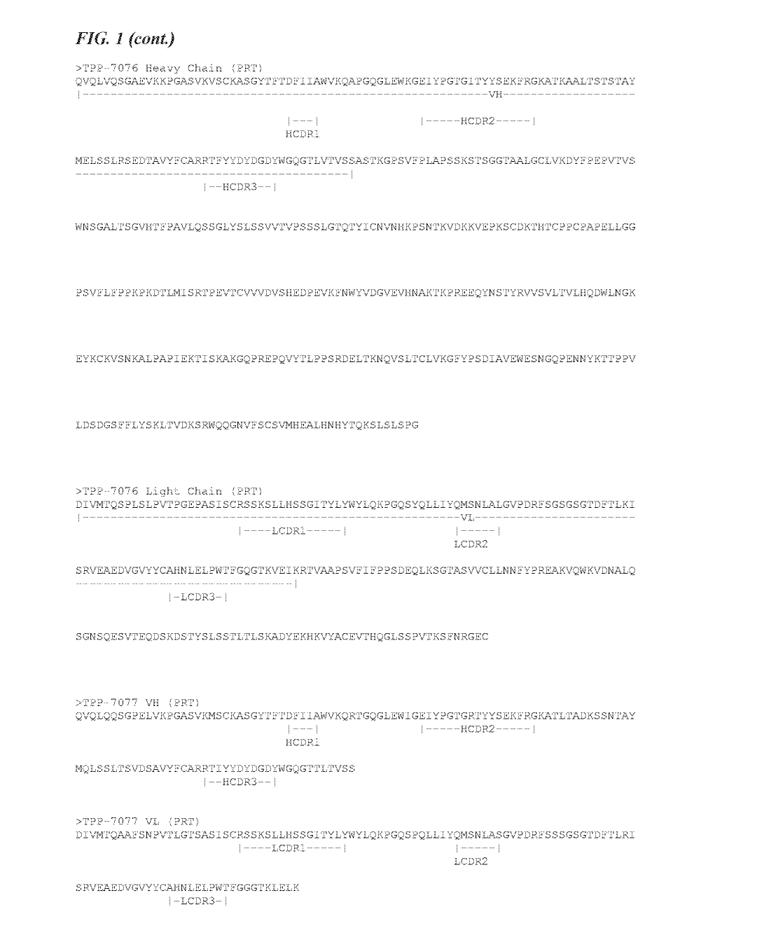

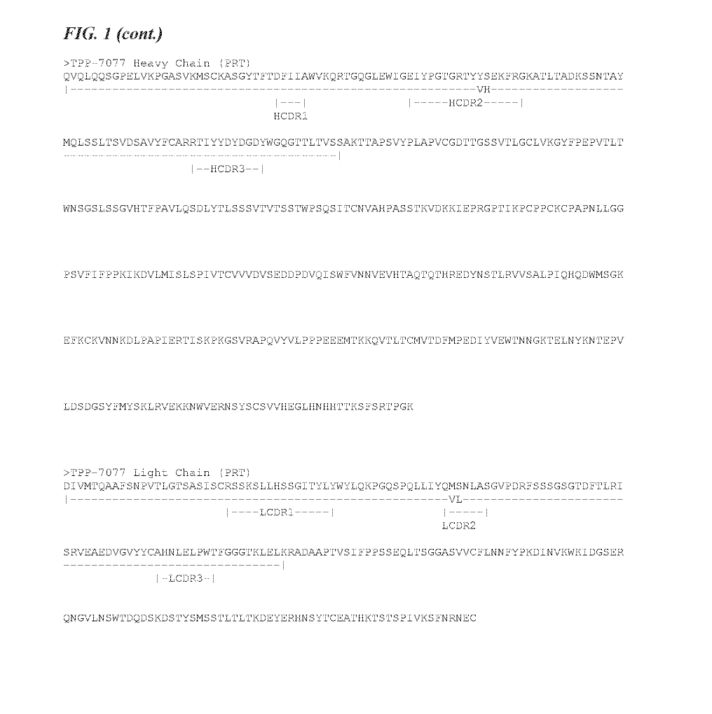

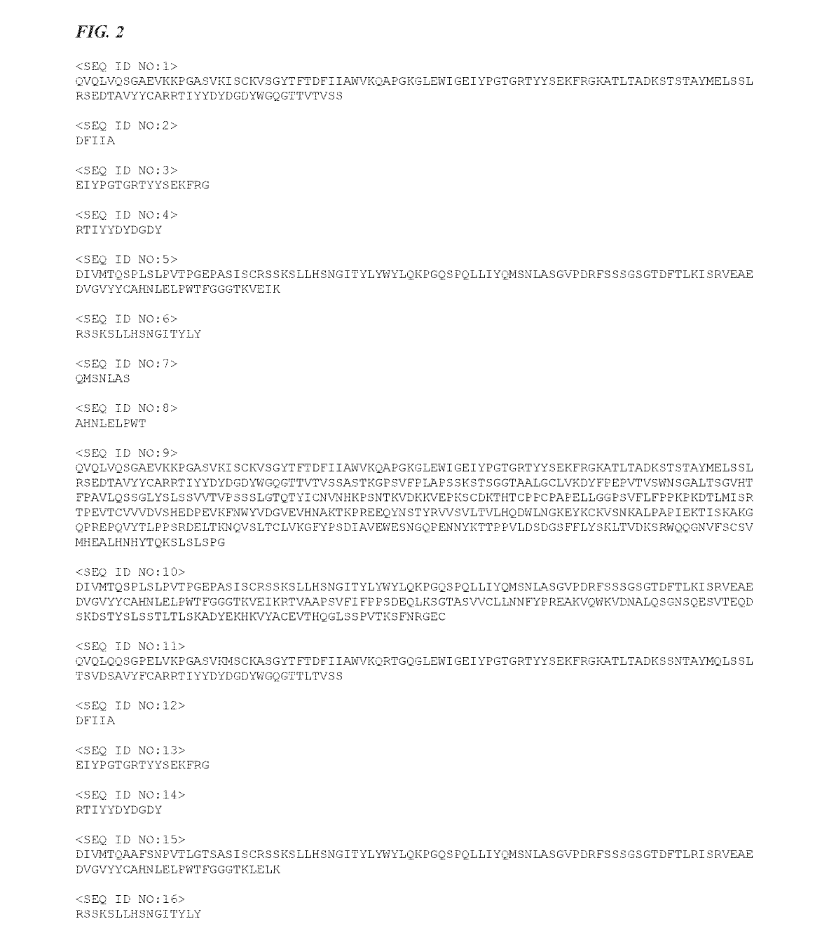

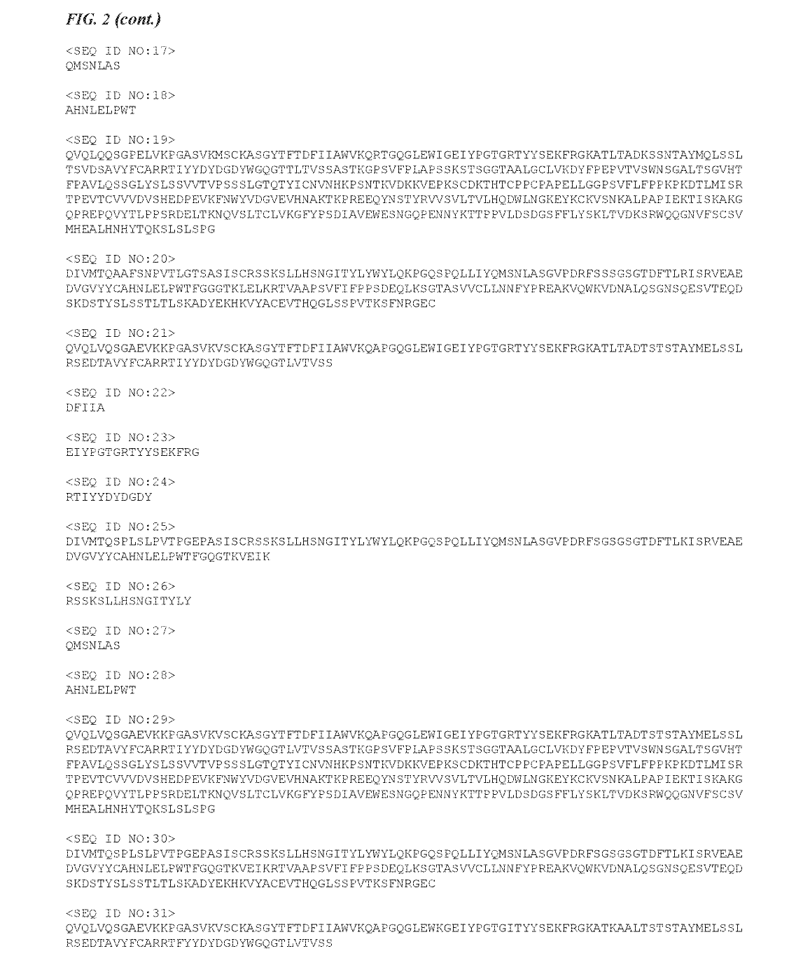

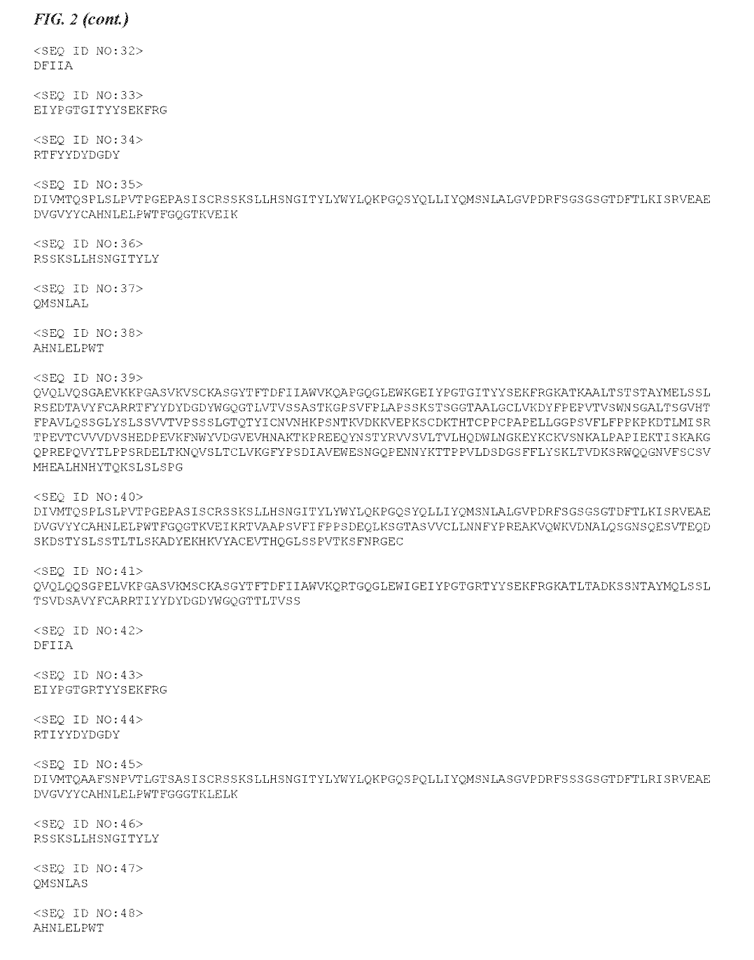

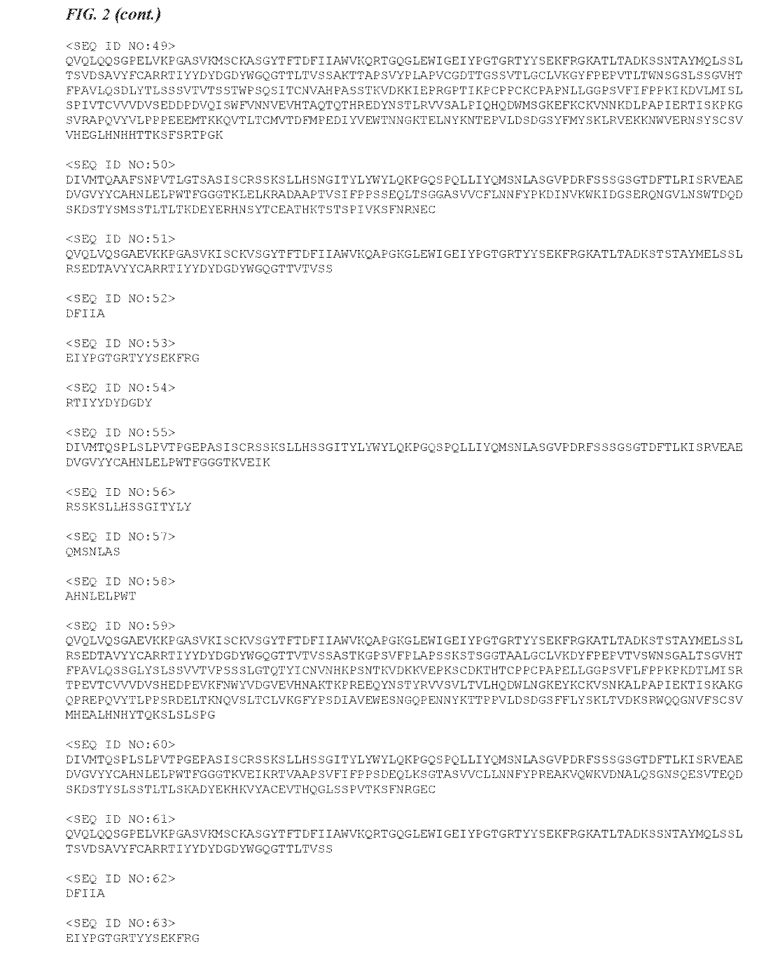

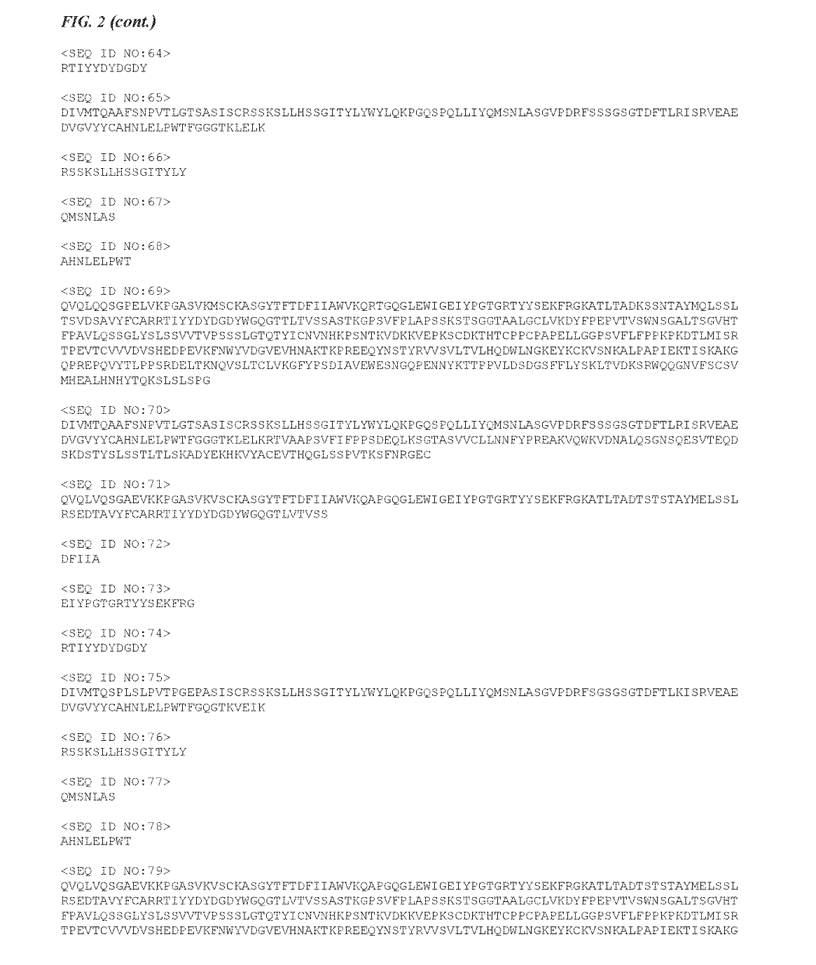

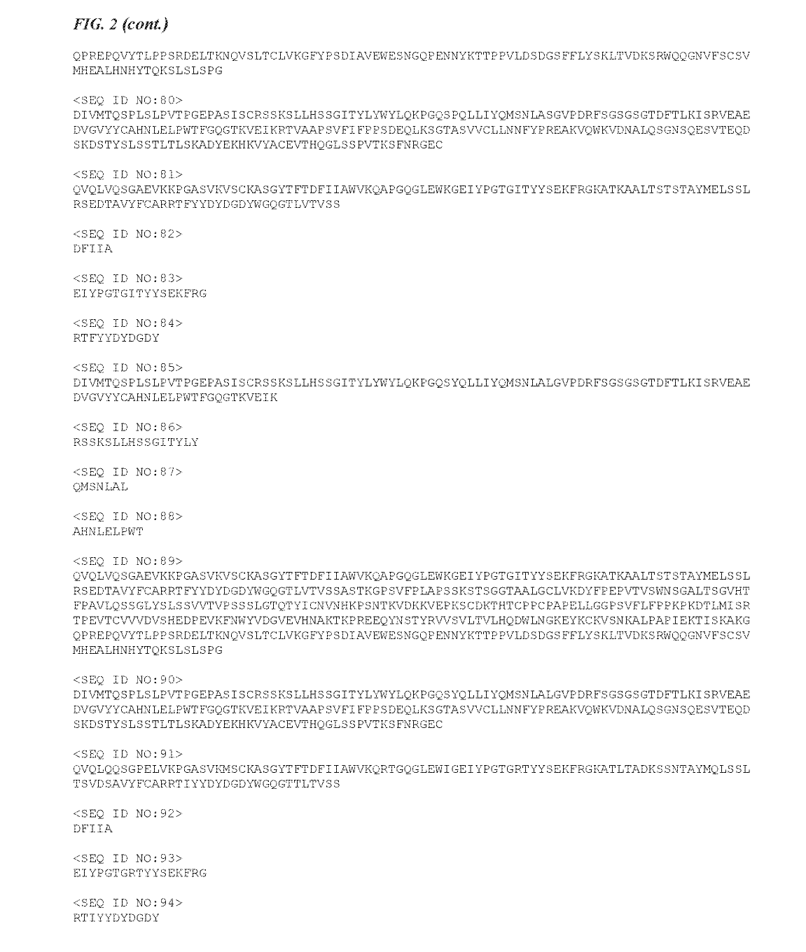

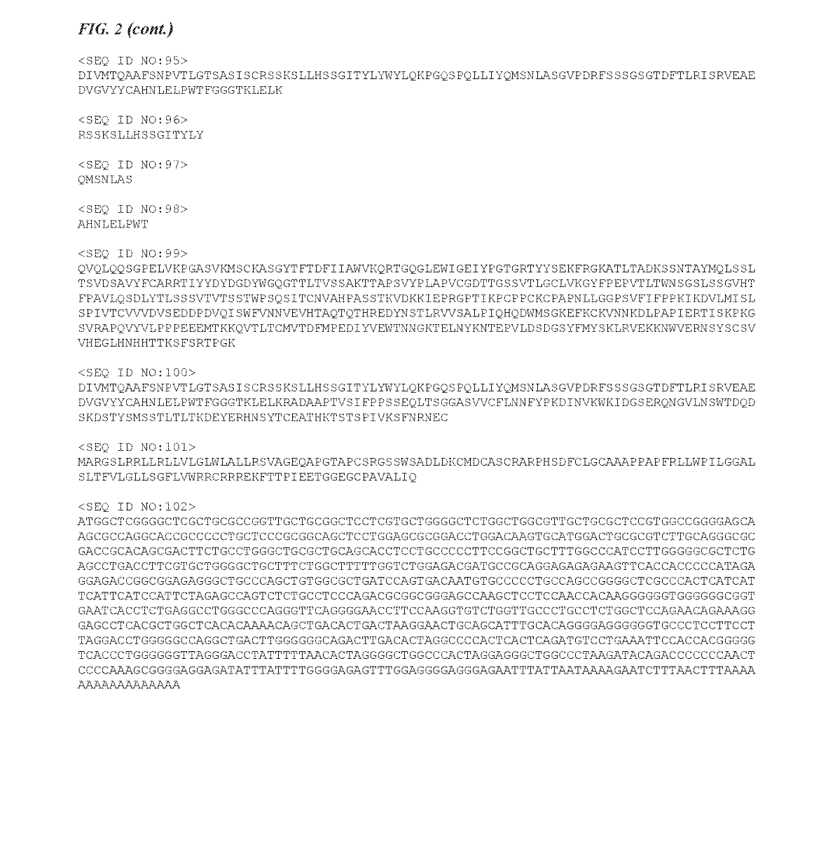

### DETAILED DESCRIPTION OF THE INVENTION

The invention provides conjugates of a moderately agonistically or non-agonistically acting anti-TWEAKR antibody such as ITEM-4 and a chimeric or humanized variant of ITEM-4 with one or more active compound molecules, the active compound molecule being a kinesin spindle protein inhibitor (KSP inhibitor) attached to the antibody via a linker L. The invention shows that a significantly improved tumour selectivity is to be observed with conjugates using an anti-TWEAKR antibody with reduced agonistic action.

The conjugate according to the invention can be represented by the general formula

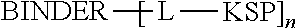

where BINDER represents the moderately agonistically or non-agonistically acting anti-TWEAKR antibody, such as e.g. ITEM-4 and chimeric or humanized variants of ITEM-4, L represents the linker, KSP represents the KSP inhibitor and n represents a number from 1 to 50, preferably from 1.2 to 20 and particularly preferably from 2 to 8. Here, n is the mean of the number of KSP inhibitor/linker conjugates per BINDER. Preferably, KSP-L has the formula (I) shown above. Furthermore, the linker is preferably attached to different amino acids of the antibody. Particular preference is given to binding to different cysteine residues of the binder. The antibody is preferably a humanized or chimeric monoclonal ITEM-4 anti-TWEAKR antibody. Particular preference is given to the humanized or chimeric ITEM-4 variants: TPP-7005, TPP-7006, TPP-7007, TPP-7053, TPP-7073, TPP-7074, TPP-7075 and TPP-7076.

Antibodies which can be used according to the invention, KSP inhibitors which can be used according to the invention and linkers which can be used according to the invention which can be used in combination without any limitation are described below. In particular, the binders represented in each case as preferred or particularly preferred can be employed in combination with the KSP inhibitors represented in each case as preferred or particularly preferred, optionally in combination with the linkers represented in each case as preferred or particularly preferred.

### KSP Inhibitors and their Binder Conjugates

### Definitions

The term “substituted” means that one or more hydrogens at the atom or group referred to is/are replaced by a selection of the group mentioned, with the proviso that the normal valency of the atom referred to is not exceeded under the present circumstances. Combinations of substituents and/or variables are admissible.

The term “optionally substituted” means that the number of substituents may be equal to or different from zero. Unless indicated otherwise, optionally substituted groups may be substituted by as many optional substituents as can be accommodated by replacing a hydrogen atom by a non-hydrogen substituent at any available carbon or nitrogen or sulphur atom. Usually, the number of optional substituents (if present) may be 1, 2, 3, 4 or 5, in particular 1, 2 or 3.

As used herein, the term “once or more than once”, for example in the definition of the substituents of the compounds of the general formulae of the present invention, means “1, 2, 3, 4 or 5, preferably 1, 2, 3 or 4, particularly preferably 1, 2 or 3, very particularly preferably 1 or 2”.

If radicals in the compounds according to the invention are substituted, the radicals may, unless indicated otherwise, be mono- or polysubstituted. In the scope of the present invention, the meanings of all radicals which occur more than once are independent of one another. Substitution by one, two or three identical or different substituents is preferred. Substitution by one substituent is particularly preferred.

### Alkyl

Alkyl represents a straight-chain or branched saturated monovalent hydrocarbon radical having 1 to 10 carbon atoms (C₁-C₁₀-alkyl), generally 1 to 6 (C₁-C₆-alkyl), preferably 1 to 4 (C₁-C₄-alkyl) and particularly preferably 1 to 3 carbon atoms (C₁-C₃-alkyl).

The following may be mentioned by way of example and as being preferred:

methyl-, ethyl-, propyl-, butyl-, pentyl-, hexyl-, isopropyl-, isobutyl-, sec-butyl, tert-butyl-, isopentyl-, 2-methylbutyl-, 1-methylbutyl-, 1-ethylpropyl-, 1,2-dimethylpropyl-, neopentyl-, 1,1-dimethylpropyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl- and 1,2-dimethylbutyl-.

Particular preference is given to a methyl-, ethyl-, propyl-, isopropyl- and tert-butyl radical.

### Heteroalkyl

Heteroalkyl represents a straight-chain and/or branched hydrocarbon chain having 1 to 10 carbon atoms which may be interrupted once or more than once by one or more of the groups —O—, —S—, —C(═O)—, —S(═O)—, —S(═O)₂—, —NR^{y}—, —NR^{y}C(═O)—, —C(═O)—NR^{y}—, —NR^{y}NR^{y}—, —S(═O)₂—NR^{y}NR^{y}—, —C(═O)—NR^{y}NR^{y}—, —CR^{x}═N—O—, and where the hydrocarbon chain including side chains, if present, may be substituted by —NH—C(═O)—NH₂, —C(═O)—OH, —OH, —NH₂,

—NH—C(═NNH₂)—, sulphonamide, sulphone, sulphoxide or sulphonic acid.

Here, R^{y} in each case represents —H, phenyl-, C₁-C₁₀-alkyl-, C₂-C₁₀-alkenyl- or C₂-C₁₀-alkynyl-, which for their part may each be substituted by —NH—C(═O)—NH₂, —C(═O)—OH, —OH, —NH₂, —NH—C(═NNH₂)—, sulphonamide, sulphone, sulphoxide or sulphonic acid.

Here, R^{x} represents —H, C₁-C₃-alkyl- or phenyl-.

### Alkenyl

Alkenyl represents a straight-chain or branched monovalent hydrocarbon chain having one or two double bonds and 2, 3, 4, 5, 6, 7, 8, 9 or 10 carbon atoms (C₂-C₁₀-alkenyl), in particular 2 or 3 carbon atoms (C₂-C₃-alkenyl), it being understood that, if the alkenyl group contains more than one double bond, the double bonds may be isolated from one another or conjugated to one another. The alkenyl group is, for example, an ethenyl (or vinyl), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1-5-dienyl group. The group is in particular vinyl or allyl.

### Alkynyl

Alkynyl represents a straight-chain or branched monovalent hydrocarbon chain having a triple bond and 2, 3, 4, 5, 6, 7, 8, 9 or 10 carbon atoms (C₂-C₁₀-alkynyl), in particular 2 or 3 carbon atoms (C₂-C₃-alkynyl). The C₂-C₆-alkynyl group is, for example, an ethynyl, prop-1-ynyl, prop-2-ynyl (or propargyl), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl- or 3,3-dimethylbut-1-ynyl group. The alkynyl group is in particular ethynyl, prop-1-ynyl or prop-2-ynyl.

### Cycloalkyl

Cycloalkyl represents a saturated monovalent mono- or bicyclic hydrocarbon radical having 3-12 carbon atoms (C₃-C₁₂-cycloalkyl).

Here, a monocyclic hydrocarbon radical represents a monovalent hydrocarbon radical having generally 3 to 10 (C₃-C₁₀-cycloalkyl), preferably 3 to 8 (C₃-C₅-cycloalkyl) and particularly preferably 3 to 7 (C₃-C₇-cycloalkyl) carbon atoms.

The following may be mentioned by way of example and as being preferred for a monocyclic hydrocarbon radical:

cyclopropyl, cyclobutyl, cyclopentyl, cyclohexyl, cycloheptyl and cyclooctyl.

Particular preference is given to cyclopropyl, cyclobutyl, cyclopentyl, cyclohexyl and cycloheptyl.

Here, a bicyclic hydrocarbon radical represents a hydrocarbon radical having generally 3 to 12 carbon atoms (C₃-C₁₂-cycloalkyl), which is to be understood as a fusion of two saturated ring systems jointly sharing two directly adjacent atoms. The following may be mentioned by way of example and as being preferred for a bicyclic hydrocarbon radical: bicyclo[2.2.0]hexyl, bicyclo[3.3.0]octyl, bicyclo[4.4.0]decyl, bicyclo[5.4.0]undecyl, bicyclo[3.2.0]heptyl, bicyclo[4.2.0]octyl, bicyclo[5.2.0]nonyl, bicyclo[6.2.0]decyl, bicyclo[4.3.0]nonyl, bicyclo[5.3.0]decyl, bicyclo[6.3.0]undecyl and bicyclo[5.4.0]undecyl.

### Heterocycloalkyl

Heterocycloalkyl represents a non-aromatic mono- or bicyclic ring system having one, two, three or four heteroatoms which may be identical or different. Heteroatoms present may be nitrogen atoms, oxygen atoms or sulphur atoms.

A monocyclic ring system in accordance with the present invention may have 3 to 8, preferably 4 to 7, particularly preferably 5 or 6, ring atoms.

The following may be mentioned by way of example and as being preferred for a heterocycloalkyl having 3 ring atoms:

aziridinyl.

The following may be mentioned by way of example and as being preferred for a heterocycloalkyl having 4 ring atoms:

azetidinyl, oxetanyl.

The following may be mentioned by way of example and as being preferred for a heterocycloalkyl having 5 ring atoms:

pyrrolidinyl, imidazolidinyl, pyrazolidinyl, pyrrolinyl, dioxolanyl and tetrahydrofuranyl.

The following may be mentioned by way of example and as being preferred for a heterocycloalkyl having 6 ring atoms:

piperidinyl, piperazinyl, morpholinyl, dioxanyl, tetrahydropyranyl and thiomorpholinyl.

The following may be mentioned by way of example and as being preferred for a heterocycloalkyl having 7 ring atoms:

azepanyl, oxepanyl, 1,3-diazepanyl, 1,4-diazepanyl.

The following may be mentioned by way of example and as being preferred for a heterocycloalkyl having 8 ring atoms:

oxocanyl, azocanyl.

From among monocyclic heterocycloalkyl, preference is given to 4- to 7-membered saturated heterocyclyl radicals having up to 2 heteroatoms from the group consisting of O, N and S. Particular preference is given to morpholinyl, piperidinyl, pyrrolidinyl and tetrahydrofuranyl.

A bicyclic ring system having one, two, three or four heteroatoms, which may be identical or different, may, in accordance with the present invention, have 6 to 12, preferably 6 to 10, ring atoms, where one, two, three or four carbon atoms may be replaced by identical or different heteroatoms from the group consisting of O, N and S.

The following may be mentioned by way of example: azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl, and also radicals derived from further possible combinations according to the definitions.

Particular preference is given to perhydrocyclopenta[c]pyrrolyl, perhydrofuro[3,2-c]pyridinyl, perhydropyrrolo[1,2-a]pyrazinyl, perhydropyrrolo[3,4-c]pyrrolyl and 3,4-methylenedioxyphenyl.

### Aryl

Aryl is a monovalent mono- or bicyclic aromatic ring system which consists of carbon atoms. Examples are naphthyl and phenyl; preference is given to phenyl or a phenyl radical.

C₆-C₁₀-Aralkyl

In the context of the invention, C₆₋₁₀-aralkyl represents a monocyclic aromatic aryl, for example phenyl, which is attached to a C₁-C₄-alkyl group.

An exemplary C₆₋₁₀-aralkyl group is benzyl.

### Heteroaryl

Heteroaryl is a monovalent monocyclic, bicyclic or tricyclic aromatic ring system having 5, 6, 8, 9, 10, 11, 12, 13 or 14 ring systems (a “5- to 14-membered heteroaryl” group), in particular 5, 6, 9 or 10 ring atoms, which contains at least one ring heteroatom and optionally one, two or three further ring heteroatoms from the group consisting of N, O and S and which is attached via a ring carbon atom or optionally (the valency permitting) via a ring nitrogen atom.

The heteroaryl group may be a 5-membered heteroaryl group such as, for example, thienyl, furyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group such as, for example, pyridinyl, pyridazinyl, pyrimidinyl, pyrazinyl or triazinyl; or a tricyclic heteroaryl group such as, for example, carbazolyl, acridinyl or phenazinyl; or a 9-membered heteroaryl group such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group such as, for example, quinolinyl, quinazolinyl, isoquinolinyl, cinnolinyl, phthalazinyl, quinoxalinyl or pteridinyl.

In general, and if not mentioned otherwise, the heteroaryl radicals include all possible isomeric forms thereof, for example tautomers and positional isomers with respect to the point of attachment to the remainder of the molecule. Thus, as an illustrative, non-including example, the term pyridinyl includes pyridin-2-yl, pyridin-3-yl and pyridin-4-yl; or the term thienyl includes thien-2-yl and thien-3-yl.

C₅-C₁₀-Heteroaryl

In the context of the invention, C₅₋₁₀-heteroaryl represents a mono- or bicyclic aromatic ring system having one, two, three or four heteroatoms which may be identical or different. Heteroatoms which may be present are: N, O, S, S(═O) and/or S(═O)₂. The binding valency may be located at any aromatic carbon atom or at a nitrogen atom.

A monocyclic heteroaryl radical in accordance with the present invention has 5 or 6 ring atoms. Preference is given to heteroaryl radicals having one or two heteroatoms. Particular preference is given here to one or two nitrogen atoms.

Heteroaryl radicals having 5 ring atoms include, for example, the rings:

thienyl, thiazolyl, furyl, pyrrolyl, oxazolyl, imidazolyl, pyrazolyl, isoxazolyl, isothiazolyl, oxadiazolyl, triazolyl, tetrazolyl and thiadiazolyl.

Heteroaryl radicals having 6 ring atoms include, for example, the rings:

pyridinyl, pyridazinyl, pyrimidinyl, pyrazinyl and triazinyl.

A bicyclic heteroaryl radical in accordance with the present invention has 9 or 10 ring atoms.

Heteroaryl radicals having 9 ring atoms include, for example, the rings:

phthalidyl, thiophthalidyl, indolyl, isoindolyl, indazolyl, benzothiazolyl, benzofuryl, benzothienyl, benzimidazolyl, benzoxazolyl, azocinyl, indolizinyl, purinyl, indolinyl.

Heteroaryl radicals having 10 ring atoms include, for example, the rings:

isoquinolinyl, quinolinyl, quinolizinyl, quinazolinyl, quinoxalinyl, cinnolinyl, phthalazinyl, 1,7- and 1,8-naphthyridinyl, pteridinyl, chromanyl.

### Heteroalkoxy

Heteroalkoxy represents a straight-chain and/or branched hydrocarbon chain having 1 to 10 carbon atoms which is attached to the remainder of the molecule via —O— and which may furthermore be interrupted once or more than once by one or more of the groups —O—, —S—, —C(═O)—, —S(═O)—, —S(═O)₂—, —NR^{y}—, —NR^{y}C(═O)—, —C(═O)—NR^{y}—, —NR^{y}NR^{y}—, —S(═O)₂—NR^{y}NR^{y}—, —C(═O)—NR^{y}NR^{y}—, —CR^{x}═N—O—, and where the hydrocarbon chain including the side chains, if present, may be substituted by —NH—C(═O)—NH₂, —C(═O)—OH, —OH, —NH₂, —NH—C(═NNH₂)—, sulphonamide, sulphone, sulphoxide or sulphonic acid.

Here, R^{y} in each case represents —H, phenyl, C₁-C₁₀-alkyl, C₂-C₁₀-alkenyl or C₂-C₁₀-alkynyl, which for their part may in each case be substituted by —NH—C(═O)—NH₂, —C(═O)—OH, —OH, —NH₂, —NH—C(═NNH₂)—, sulphonamide, sulphone, sulphoxide or sulphonic acid.

Here, R^{x} represents —H, C₁-C₃-alkyl or phenyl.

In the context of the invention, halogen or halogen atom represents fluorine (—F), chlorine (—Cl), bromine (—Br) or iodine (—I).

Fluoroalkyl, fluoroalkenyl and fluoroalkynyl mean that alkyl, alkenyl and alkynyl may be mono- or polysubstituted by fluorine.

The conjugation of the KSP inhibitor to the antibody can take place chemically by various routes, as shown in an exemplary manner in Schemes 20 to 31 in the examples. In particular, it is optionally possible to modify the low-molecular weight KSP inhibitor for the conjugation to the linker, for example by introducing protective groups or leaving groups to facilitate substitution (such that in the reaction said leaving group, and not a hydrogen atom, is substituted by the linker). The KSP inhibitor-linker molecule obtained in this manner (where the linker has a reactive group for coupling to the binder) can then be reacted with the binder to give a binder conjugate according to the invention. In the experimental section, this procedure is illustrated in an exemplary manner by a large number of examples.

Other particularly preferred compounds have the formula (I) or (Ia) below:

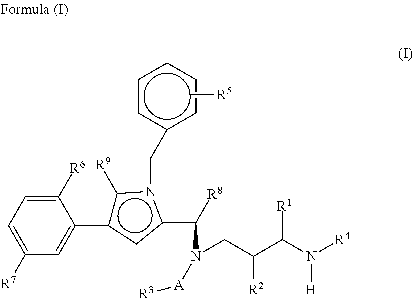

where
R¹ represents H, -L-#1, -MOD or —(CH₂)₀₋₃Z, where Z represents —H, —NHY³, —OY³, —SY³, halogen, —CO—NY¹Y² or —CO—OY³,
where Y¹ and Y² independently of one another represent H, NH₂, —(CH2CH₂O)₀₋₃—(CH₂)₀₋₃Z′ (e.g. —(CH₂)₀₋₃Z′) or —CH(CH₂W)Z′, and Y³ represents H or —(CH₂)₀₋₃Z′, where Z′ represents H, NH₂, SO₃H, COOH, —NH—CO—CH₂—CH₂—CH(NH₂)COOH or —(CO—NH—CHY⁴)₁₋₃COOH, where W represents H or OH,
where Y⁴ represents straight-chain or branched C₁₋₆ alkyl which is optionally substituted by —NHCONH₂, or represents aryl or benzyl which are optionally substituted by —NH₂;
R² represents H, -MOD, —CO—CHY⁴—NHY⁵ or —(CH₂)₀₋₃Z,
where Z represents —H, halogen, —OY³, —SY³, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where Y⁴ represents straight-chain or branched C₁₋₆-alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl;
R⁴ represents H, -L-#1, -SG_{lys}-(CO)₀₋₁—R^{4′}, —CO—CHY⁴—NHY⁵ or —(CH₂)₀₋₃Z,
where SG_{lys} represents a group which can be cleaved by a lysosomal enzyme, in particular a group consisting of a di- or tripeptide, R^{4′} represents a C₁₋₁₀-alkyl, C₅₋₁₀-aryl or C₆₋₁₀-aralkyl, C₅₋₁₀-heteroalkyl, C₁₋₁₀-alkyl-O—C₆₋₁₀-aryl, C₅₋₁₀-heterocycloalkyl, heteroaryl, heteroarylalkyl, heteroarylalkoxy, C₁₋₁₀-alkoxy, C₆₋₁₀-aryloxy or C₆₋₁₀-aralkoxy, C₅₋₁₀-heteroaralkoxy, C₁₋₁₀-alkyl-O—C₆₋₁₀-aryloxy, C₅₋₁₀-heterocycloalkoxy group which may be mono- or polysubstituted by —NH₂, —NH-alkyl, —N(alkyl)₂, NH—CO-alkyl, N(alkyl)-COalkyl, —SO₃H, —SO₂NH₂, —SO₂—N(alkyl)₂, —COOH, —CONH₂, —CON(alkyl)₂ or —OH, —H or a group —Oₓ—(CH₂CH₂O)_{y}—R^{4″} (where x represents 0 or 1 and v represents a number from 1 to 10 and R^{4″} represents —H, -alkyl (preferably C₁₋₁₂-alkyl), —CH₂—COOH, —CH₂—CH₂—COOH or —CH₂—CH₂—NH₂);
where Z represents —H, halogen, —OY³, —SY³, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where Y⁴ represents straight-chain or branched C₁₋₆-alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl; or R² and R⁴ together (with formation of a pyrrolidine ring) represent —CH₂—CHR¹¹— or —CHR¹¹—CH₂—, where R¹¹ represents H, NH₂, SO₃H, COOH, SH, halogen (in particular F or Cl), C₁₋₄-alkyl, C₁₋₄-haloalkyl, C₁₋₄-alkoxy, hydroxyl-substituted C₁₋₄-alkyl, COO(C₁₋₄-alkyl) or OH;
A represents CO, SO, SO₂, SO₂NH or CNNH₂;
R³ represents -L-#1, -MOD or an optionally substituted alkyl, cycloalkyl, aryl, heteroaryl, heteroalkyl, heterocycloalkyl group, preferably a C₁₋₁₀-alkyl, C₆₋₁₀-aryl or C₆₋₁₀-aralkyl, C₅₋₁₀-heteroalkyl, C₁₋₁₀-alkyl-O—C₆₋₁₀-aryl or C₅₋₁₀-heterocycloalkyl group which may be substituted by 1-3 —OH groups, 1-3 halogen atoms, 1-3 halogenated alkyl groups (each having 1-3 halogen atoms), 1-3 O-alkyl groups, 1-3 —SH groups, 1-3 —S-alkyl groups, 1-3 —O—CO-alkyl groups, 1-3 —O—CO—NH-alkyl groups, 1-3 —NH—CO-alkyl groups, 1-3 —NH—CO—NH-alkyl groups, 1-3 —S(O)ₙ-alkyl groups, 1-3 —SO₂—NH-alkyl groups, 1-3 —NH-alkyl groups, 1-3 —N(alkyl)₂ groups, 1-3 —NH((CH₂CH₂O)1-20H) groups, 1-3 —NH2 groups or 1-3 —(CH2)₀₋₃Z groups, where n represents 0, 1 or 2, Z represents —H, halogen, —OY³, —SY³, —NHY³, —CO—NY¹Y² or —CO—OY³, where Y¹ and Y² independently of one another represent H, NH₂ or —(CH₂)₀₋₃Z′ and Y³ represents H, —(CH₂)₀₋₃—CH(NHCOCH₃)Z′, —(CH₂)₀₋₃—CH(NH₂)Z′ or —(CH₂)₀₋₃Z′, where Z′ represents H, SO₃H, NH₂ or COOH (where “alkyl” is preferably C₁₋₁₀-alkyl);
R⁵ represents H, -MOD, NH₂, NO₂, halogen (in particular F, Cl, Br), —CN, CF₃, —OCF₃, —CH₂F, —CH₂F, SH or —(CH₂)₀₋₃Z, where Z represents —H, —OY³, —SY³, halogen, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
R⁶ and R⁷ independently of one another represent H, cyano, (optionally fluorinated) C₁₋₁₀-alkyl, (optionally fluorinated) C₂₋₁₀-alkenyl, (optionally fluorinated) C₂₋₁₀-alkynyl, hydroxy, NO₂, NH₂, COOH or halogen (in particular F, Cl, Br),
R⁸ represents (optionally fluorinated) C₁₋₁₀-alkyl, (optionally fluorinated) C₂₋₁₀-alkenyl, (optionally fluorinated) C₂₋₁₀-alkynyl, (optionally fluorinated) C₄₋₁₀-cycloalkyl or —(CH₂)₀₋₂—(HZ²), where HZ² represents a 4- to 7-membered heterocycle having up to two heteroatoms selected from the group consisting of N, O and S (preferably oxetane), where each of these groups may be substituted by —OH, CO₂H or NH₂;
where one of the substituents R¹, R³ and R⁴ represents -L-#1,
L represents the linker and #1 represents the bond to the antibody,
R⁹ represents H, F, CH₃, CF₃, CH₂F or CHF₂;
where -MOD represents —(NR¹⁰)ₙ-(G1)ₒ-G2-G3, where
R¹⁰ represents H or C₁-C₃-alkyl;
G1 represents —NHCO—, —CONH— or

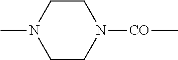

(where, if G1 represents —NHCO— or

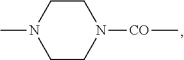

R¹⁰ does not represent NH₂);
n represents 0 or 1;
o represents 0 or 1; and
G2 represents a straight-chain and/or branched hydrocarbon group which has 1 to 10 carbon atoms and which may be interrupted once or more than once by one or more of the groups —O—, —S—, —SO—, SO₂, -NRy-, -NRyCO—, CONRy-, -NRyNRy-, —SO₂NRyNRy-, -CONRyNRy- (where R^{y} represents H, phenyl, C1-C10-alkyl, C2-C10-alkenyl or C2-C10-alkynyl, each of which may be substituted by NHCONH₂, —COOH, —OH, —NH₂, NH—CNNH₂, sulphonamide, sulphone, sulphoxide or sulphonic acid), —CO—, or —CR^{x}═N—O— (where Rx represents H, C1-C3-alkyl or phenyl), where the hydrocarbon chain including any side chains may be substituted by —NHCONH₂, —COOH, —OH, —NH₂, NH—CNNH₂, sulphonamide, sulphone, sulphoxide or sulphonic acid, where G3 represents —H or —COOH, and where the group -MOD preferably has at least one group —COOH;
and the salts, solvates, salts of the solvates and epimers thereof.

In a preferred embodiment of the formula (I), one of the substituents R¹ or R³ represents -L-#1. In this embodiment, it is particularly preferred if R⁴ represents H or -SG_{lys}-(CO)₀₋₁—R^{4′}, where SG_{lys} and R^{4′} have the same meaning as above. In another preferred embodiment of the formula (I), the substituent R⁴ represents -L-#1, where the linker is a linker which can be cleaved at the nitrogen atom which binds to R⁴, so that a primary amino group is present after cleavage (corresponds to R⁴=H). Such cleavable groups are described in detail below.

If R¹ does not represent H, the carbon atom to which R¹ binds is a stereocentre which may be present in the L and/or D configuration, preferably in the L configuration.

If R² does not represent H, the carbon atom to which R² binds is a stereocentre which may be present in the L and/or D configuration.

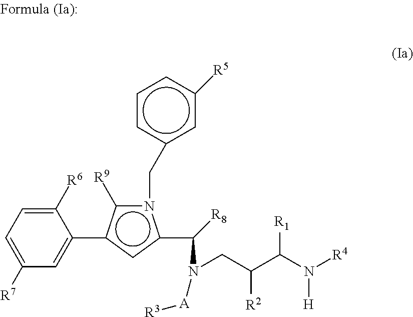

where
R¹ represents H, -L-#1 or —(CH₂)₀₋₃Z, where Z represents —H, —NHY³, —OY³, —SY³, halogen, —CO—NY¹Y² or —CO—OY³,
where Y¹ and Y² independently of one another represent H, NH₂, —(CH2CH₂O)₀₋₃—(CH₂)₀₋₃Z′ (e.g. —(CH₂)₀₋₃Z′) or —CH(CH₂W)Z′, and Y³ represents H or —(CH₂)₀₋₃Z′, where Z′ represents H, NH₂, SO₃H, COOH, —NH—CO—CH₂—CH₂—CH(NH₂)COOH or —(CO—NH—CHY⁴)₁₋₃COOH, where W represents H or OH;
where Y⁴ represents straight-chain or branched C₁₋₆ alkyl which is optionally substituted by —NHCONH₂, or represents aryl or benzyl which are optionally substituted by —NH₂.
R² and R⁴ independently of one another represent H, -SG_{lys}-(CO)₀₋₁—R^{4′}, —CO—CHY⁴—NHY⁵ or —(CH₂)₀₋₃Z,
where SG_{lys} represents a group which can be cleaved by a lysosomal enzyme, in particular a group consisting of a di- or tripeptide, R^{4′} represents a C₁₋₁₀-alkyl, C₅₋₁₀-aryl or C₆₋₁₀-aralkyl, C₅₋₁₀-heteroalkyl, C₁₋₁₀-alkyl-O—C₆₋₁₀-aryl, C₅₋₁₀-heterocycloalkyl, heteroaryl, heteroarylalkyl, heteroarylalkoxy, C₁₋₁₀-alkoxy, C₆₋₁₀-aryloxy or C₆₋₁₀-aralkoxy, C₅₋₁₀-heteroaralkoxy, C₁₋₁₀-alkyl-O—C₆₋₁₀-aryloxy, C₅₋₁₀-heterocycloalkoxy group, each of which may be mono- or polysubstituted by —NH₂, —NH-alkyl, —N(alkyl)₂, NH—CO-alkyl, N(alkyl)-COalkyl, —SO₃H, —SO₂NH₂, —SO₂—N(alkyl)₂, —COOH, —CONH₂, —CON(alkyl)₂ or —OH, —H or a group —Oₓ—(CH₂CH₂O)_{y}—R^{4″} (where x represents 0 or 1 and v represents a number from 1 to 10 and of R^{4″} represents —H, -alkyl (preferably C₁₋₁₂-alkyl), —CH₂—COOH, —CH₂—CH₂—COOH or —CH₂—CH₂—NH₂);
or R² and R⁴ together represent (with formation of a pyrrolidine ring) —CH₂—CHR¹¹— or —CHR¹¹—CH₂—, where R¹¹ represents H, NH₂, SO₃H, COOH, SH, halogen (in particular F or Cl), C₁₋₄-alkyl, C₁₋₄-haloalkyl, C₁₋₄-alkoxy, hydroxyl-substituted C₁₋₄-alkyl, COO(C₁₋₄-alkyl) or OH; or R² represents H, —CO—CHY⁴—NHY⁵ or —(CH₂)₀₋₃Z and R⁴ represents -L-#1 darstellt, and where Z represents —H, halogen, —OY³, —SY³, —NHY³, —CO—NY₁Y₂ or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where Y⁴ independently of one another represents straight-chain or branched C₁₋₆-alkyl which is optionally substituted by —NHCONH₂ or represents aryl or benzyl which are optionally substituted by —NH₂, where Y⁴ represents straight-chain or branched C₁₋₆-alkyl which is optionally substituted by —NHCONH₂ or represents aryl or benzyl which are optionally substituted by —NH₂ and Y⁵ represents H or —CO—CHY⁶—NH₂, where Y⁶ represents straight-chain or branched C₁₋₆-alkyl;
A represents CO, SO, SO₂, SO₂NH or CNNH₂;
R³ represents an optionally substituted alkyl, aryl, heteroaryl, heteroalkyl, heterocycloalkyl group, preferably -L-#1 or a C₁₋₁₀-alkyl, C₆₋₁₀-aryl or C₆₋₁₀-aralkyl, C₅₋₁₀-heteroalkyl, C₁₋₁₀-alkyl-O—C₆₋₁₀-aryl or C₅₋₁₀-heterocycloalkyl group which may be substituted by 1-3 —OH groups, 1-3 halogen atoms, 1-3 halogenated alkyl groups (each having 1-3 halogen atoms), 1-3 O-alkyl groups, 1-3 —SH groups, 1-3 —S-alkyl groups, 1-3 —O—CO-alkyl groups, 1-3 —O—CO—NH-alkyl groups, 1-3 —NH—CO-alkyl groups, 1-3 —NH—CO—NH-alkyl groups, 1-3 —S(O)ₙ-alkyl groups, 1-3 —SO₂—NH-alkyl groups, 1-3 —NH-alkyl groups, 1-3 —N(alkyl)₂ groups, 1-3 —NH₂ groups or 1-3 —(CH₂)₀₋₃Z groups, where n represents 0, 1 or 2, Z represents —H, halogen, —OY³, —SY³, —NHY³, —CO—NY¹Y² or —CO—OY³, where Y¹ and Y² independently of one another represent H, NH₂ or —(CH₂)₀₋₃Z′ and Y³ represents H, —(CH₂)₀₋₃—CH(NHCOCH₃)Z′, —(CH₂)₀₋₃—CH(NH₂)Z′ or —(CH₂)₀₋₃Z′, where Z′ represents H, SO₃H, NH₂ or COOH
(where “alkyl” preferably represents C₁₋₁₀-alkyl);
R⁵ represents H, F, NH₂, NO₂, halogen, SH or —(CH₂)₀₋₃Z, where Z represents —H, halogen, —OY³, —SY³, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
R⁶ and R⁷ independently of one another represent H, cyano, (optionally fluorinated) C₁₋₁₀-alkyl, (optionally fluorinated) C₂₋₁₀-alkenyl, (optionally fluorinated) C₂₋₁₀-alkynyl, hydroxy or halogen,
R⁸ represents (optionally fluorinated) C₁₋₁₀-alkyl, (optionally fluorinated) C₄₋₁₀-cycloalkyl or optionally substituted oxetane; and
R⁹ represents H, F, CH₃, CF₃, CH₂F or CHF₂;
and the salts, solvates, salts of the solvates and epimers thereof.

By substitution of a hydrogen atom at R¹, R³ or R⁴, it is possible to attach a compound of the formula (I) or (Ia) in which none of the substituents R¹, R³ and R⁴ represents -L-#1 to a linker in a manner known to the person skilled in the art. This gives conjugates of the formula (I) or (Ia) where one of the substituents R¹, R³ or R⁴ represents -L-#1, L represents the linker and #1 represents the bond to the antibody. If the KSP inhibitor according to formula (I) or (Ia) is conjugated with a binder, one of the substituents R¹, R³ or R⁴ thus represents -L-#1, where L represents the linker and #1 represents the bond to the antibody. That is, in the case of the conjugates one of the substituents R¹, R³ or R⁴ represents -L-#1, where -L-#1 represents the bond to the antibody. The binder is preferably a human, humanized or chimeric monoclonal antibody or an antigen-binding fragment thereof. In a preferred embodiment of the formula (I) or (Ia), one of the substituents R¹ or R³ represents -L-#1. In this embodiment, it is particularly preferred if R⁴ represents H or -SG_{lys}-(CO)₀₋₁—R^{4′,} where SG_{lys} and R^{4′} have the same meaning as above. In another preferred embodiment of the formula (I), the substituent R⁴ represents -L-#1, where the linker is a linker which can be cleaved at the nitrogen atom which binds to R⁴, so that a primary amino group is present after cleavage (corresponds to R⁴=H). Such cleavable groups are described in detail below. Preference is given to moderately agonistically or non-agonistically acting anti-TWEAKR antibodies. The antibody is preferably a humanized or chimeric monoclonal ITEM-4 anti-TWEAKR antibody. Particular preference is given to the humanized or chimeric ITEM-4 variants: TPP-7005, TPP-7006, TPP-7007, TPP-7053, TPP-7073, TPP-7074, TPP-7075 and TPP-7076.

Instead of -L-#1, it is also possible for the group -L-#3 to be present in the compound, where L represents the linker and #3 represents the reactive group for binding to the antibody. Compounds comprising -L-#3 are reactive compounds which react with the antibody. #3 is preferably a group which reacts with an amino or thiol group with formation of a covalent bond, preferably with the cysteine residue in a protein. The cysteine residue in a protein may be present naturally in the protein, may be introduced by biochemical methods or, preferably, may be generated by prior reduction of disulphides of the binder.

For A, preference is given to CO (carbonyl).

Preferred for R¹ are -L-#1, H, —COOH, —CONHNH₂, —(CH₂)₁₋₃NH₂, —CONZ″(CH₂)₁₋₃ NH₂ and —CONZ″CH₂COOH, where Z″ represents H or NH₂.

Preferred for R² and R⁴ is H, or R² and R⁴ together (with formation of a pyrrolidine ring) represent —CH₂—CHR¹¹— or —CHR¹¹—CH₂—, where R¹¹ represents H or F. Also preferred for R⁴ is -L-#1, where -L-#1 is a cleavable linker, preferably a linker which can be cleaved intracellularly by enzymes.

Preferred for R³ is -L-#1 or C₁₋₁₀-alkyl-, which may optionally be substituted by —OH, O-alkyl, SH, S-alkyl, O—CO-alkyl, O—CO—NH-alkyl, NH—CO-alkyl, NH—CO—NH-alkyl, S(O)ₙ-alkyl, SO₂—NH— alkyl, NH-alkyl, N(alkyl)₂ or NH₂ (where alkyl is preferably C₁₋₃-alkyl).

Preferred for R⁵ is H or F.

Preferred for R⁶ and R⁷, independently of one another, are H, (optionally fluorinated) C₁₋₃-alkyl, (optionally fluorinated) C₂₋₄-alkenyl, (optionally fluorinated) C₂₋₄-alkynyl, hydroxy or halogen.

Preferred for R⁸ is a branched C₁₋₅-alkyl group, in particular a group of the formula —C(CH₃)₂—(CH₂)₀₋₂—R_{y}, where R_{y} represents —H, —OH, CO₂H or NH₂, or an (optionally fluorinated) C₅₋₇-cycloalkyl. Particular preference is given to a group of the formula —C(CH₃)₃ or a cyclohexyl group.

Preferred for R⁹ is H or F.

Especially preferred are compounds of the formula (I) or (Ia) in which

A represents CO (carbonyl);
R¹ represents H, -L-#1, —COOH, —CONHNH₂, —(CH₂)₁₋₃NH₂, —CONZ″(CH₂)₁₋₃ NH₂ or —CONZ″CH₂COOH, where Z″ represents H or NH₂;
R² and R⁴ represent H or R² and R⁴ together (with formation of a pyrrolidine ring) represent —CH₂—CHR¹¹— or —CHR¹¹—CH₂—, where R¹¹ represents H or F; or R⁴ represents -L-#1 and R² represents H;
R³ represents -L-#1 or a phenyl group which may be mono- or polysubstituted by halogen (in particular F) or optionally fluorinated C₁₋₃-alkyl, or represents an optionally fluorinated C₁₋₁₀-alkyl group which may optionally be substituted by —OY⁴, —SY⁴, —O—CO—Y⁴, —O—CO—NH—Y⁴, NH—CO—Y⁴, —NH—CO—NH—Y⁴, S(O)—Y⁴ (where n represents 0, 1 or 2), —SO₂—NH—Y⁴, NH—Y⁴ or N(Y⁴)₂, where Y⁴ represents H, phenyl (optionally mono- or polysubstituted by halogen (in particular F) or optionally fluorinated C₁₋₃-alkyl), or alkyl (where the alkyl group may be substituted by —OH, —COOH, and/or —NHCO—C₁₋₃-alkyl and where alkyl preferably represents C₁₋₃-alkyl);
where particularly preferably R³ may be substituted by —OH, O-alkyl, SH, S-alkyl, O—CO-alkyl, O—CO—NH-alkyl, NH—CO-alkyl, NH—CO—NH-alkyl, S(O)ₙ-alkyl, SO₂—NH-alkyl, NH-alkyl, N(alkyl)₂ or NH₂ (where alkyl preferably means C₁₋₃-alkyl);
R⁵ represents H or F;
R⁶ and R⁷ independently of one another represent H, (optionally fluorinated) C₁₋₃-alkyl, (optionally fluorinated) C₂₋₄-alkenyl, (optionally fluorinated) C₂₋₄-alkynyl, hydroxy or halogen;
R⁸ represents a branched C₁₋₅-alkyl group or cyclohexyl; and
R⁹ represents H or F.

Furthermore, it is preferred when (alone or in combination)

R¹ represents -L-#1, COOH or H,
R² and R⁴ represent H or R² and R⁴ together (with formation of a pyrrolidine ring) represent —CH₂—
CHR¹¹— or —CHR¹¹—CH₂—, where R¹¹ represents H, or R⁴ represents -L-#1 and R² represents H;
A represents CO,
R³ represents —(CH₂)OH, —CH(CH₃)OH, —CH₂SCH₂CH(COOH)NHCOCH₃, —CH(CH₃)OCH₃, a phenyl group which may be substituted by 1-3 halogen atoms, 1-3 amino groups or 1-3 alkyl groups (which may optionally be halogenated), or represents -L-#1,
R⁵ represents H,
R⁶ and R⁷ independently of one another represent H, C₁₋₃-alkyl or halogen; in particular, R⁶ and R⁷ represent F;
R⁸ represents C₁₋₄-alkyl (preferably tert-butyl) or cyclohexyl; and/or
R⁹ represents H.

Additionally, in accordance with the invention it is preferred when

R¹ represents -L-#1, COOH or H,
R² and R⁴ represent H or R² and R⁴ together (with formation of a pyrrolidine ring) represent —CH₂—CHR¹¹— or —CHR¹¹—CH₂—, where R¹¹ represents H,
A represents CO,
R³ represents —(CH₂)OH, —CH(CH₃)OH, —CH₂SCH₂CH(COOH)NHCOCH₃, —CH(CH₃)OCH₃, a phenyl group which may be substituted by 1-3 halogen atoms, 1-3 amino groups or 1-3 alkyl groups (which may optionally be halogenated), or represents -L-#1,
R⁵ represents H,
R⁶ and R⁷ independently of one another represent H, C₁₋₃-alkyl or halogen; in particular, R⁶ and R⁷ represent F;
R⁸ represents C₁₋₄-alkyl (preferably tert-butyl); and
R⁹ represents H.

Other particularly preferred compounds have the formula (II) or (IIa) below:

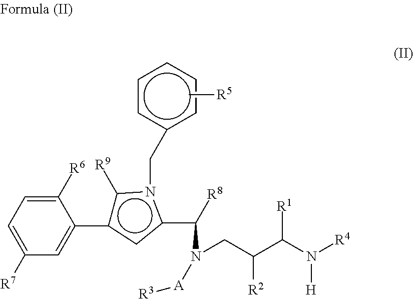

where
R¹ represents H, -L-BINDER, -MOD or —(CH₂)₀₋₃Z, where Z represents —H, —NHY³, —OY³, —SY³, halogen, —CO—NY¹Y² or —CO—OY³,
where Y¹ and Y² independently of one another represent H, NH₂, —(CH₂CH₂O)₀₋₃—(CH₂)₀₋₃Z′ (e.g. —(CH₂)₀₋₃Z′) or —CH(CH₂W)Z′, and Y³ represents H or —(CH₂)₀₋₃Z′, where Z′ represents H, NH₂, SO₃H, —COOH, —NH—CO—CH₂—CH₂—CH(NH₂)COOH or —(CO—NH—CHY⁴)₁₋₃COOH, where W represents H or OH,
where Y⁴ represents straight-chain or branched C₁₋₆ alkyl which is optionally substituted by —NHCONH₂, or represents aryl or benzyl which are optionally substituted by —NH₂;
R² represents H, -MOD, —CO—CHY⁴—NHY⁵ or —(CH₂)₀₋₃Z, where Y⁴ represents straight-chain or branched C₁₋₆ alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl,
where Z represents —H, halogen, —OY³, —SY³, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
R⁴ represents H, -L-BINDER, -SG_{lys}-(CO)₀₋₁—R^{4′}, —CO—CHY⁴—NHY⁵ or —(CH₂)₀₋₃Z, where SG_{lys} represents a group which can be cleaved by a lysosomal enzyme, in particular a group consisting of a di- or tripeptide, R^{4′} represents a C₁₋₁₀-alkyl, C₅₋₁₀-aryl or C₆₋₁₀-aralkyl, C₅₋₁₀-hetero alkyl, C₁₋₁₀-alkyl-O—C₆₋₁₀-aryl, C₅₋₁₀-heterocycloalkyl, heteroaryl, heteroarylalkyl, heteroarylalkoxy, C₁₋₁₀-alkoxy, C₆₋₁₀-aryloxy or C₆₋₁₀-aralkoxy, C₅₋₁₀-heteroaralkoxy, C₁₋₁₀-alkyl-O—C₆₋₁₀-aryloxy, C₅₋₁₀-heterocycloalkoxy group which may be mono- or polysubstituted by —NH₂, —NH-alkyl, —N(alkyl)₂, NH—CO-alkyl, N(alkyl)-COalkyl, —SO₃H, —SO₂NH₂, —SO₂—N(alkyl)₂, —COOH, —CONH₂, —CON(alkyl)₂ or —OH, —H or a group —Oₓ— (CH₂CH₂O)_{y}—R^{4″} (where x represents 0 or 1 and v represents a number from 1 to 10 and R^{4″} represents —H, -alkyl (preferably C₁₋₁₂-alkyl), —CH₂—COOH, —CH₂—CH₂—COOH or —CH₂—CH₂—NH₂);
where Z represents —H, halogen, —OY³, —SY³, NHY³, —CO—NY¹Y² or —CO—OY³, where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where Y⁴ represents straight-chain or branched C₁₋₆-alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl;
or R² and R⁴ together (with formation of a pyrrolidine ring) represent —CH₂—CHR¹¹— or —CHR¹¹—CH₂—, where R¹¹ represents H, NH₂, SO₃H, COOH, SH, halogen (in particular F or Cl), C₁₋₄-alkyl, C₁₋₄-haloalkyl, C₁₋₄-alkoxy, hydroxyl-substituted C₁₋₄-alkyl, COO(C₁₋₄-alkyl) or OH;
A represents CO, SO, SO₂, SO₂NH or CNNH₂;
R³ represents -L-BINDER, -MOD or an optionally substituted alkyl, cycloalkyl, aryl, heteroaryl, heteroalkyl, heterocycloalkyl group, preferably -L-BINDER or a C₁₋₁₀-alkyl, C₆₋₁₀-aryl or C₆₋₁₀-aralkyl, C₅₋₁₀-heteroalkyl, C₁₋₁₀-alkyl-O—C₆₋₁₀-aryl or C₅₋₁₀-heterocycloalkyl group which may be substituted by 1-3 —OH groups, 1-3 halogen atoms, 1-3 halogenated alkyl groups (each having 1-3 halogen atoms), 1-3 O-alkyl groups, 1-3 —SH groups, 1-3 —S-alkyl groups, 1-3 —O—CO-alkyl groups, 1-3 —O—CO—NH-alkyl groups, 1-3 —NH—CO-alkyl groups, 1-3 —NH—CO—NH-alkyl groups, 1-3 —S(O)ₙ-alkyl groups, 1-3 —SO₂—NH-alkyl groups, 1-3 —NH-alkyl groups, 1-3 —N(alkyl)₂ groups, 1-3 —NH₂ groups or 1-3 —(CH₂)₀₋₃Z groups, where Z represents —H, halogen, —OY³, —SY³, —NHY³, —CO—NY¹Y² or —CO—OY³, where Y¹ and Y² independently of one another represent H, NH₂ or —(CH₂)₀₋₃Z′ and Y³ represents H, —(CH₂)₀₋₃—CH(NHCOCH₃)Z′, —(CH₂)₀₋₃—CH(NH₂)Z′ or —(CH₂)₀₋₃Z′, where Z′ represents H, SO₃H, NH₂ or COOH
(where “alkyl” preferably represents C₁₋₁₀-alkyl);
R⁵ represents H, NH₂, NO₂, halogen (in particular F, Cl, Br), —CN, CF₃, —OCF₃, —CH₂F, —CH₂F, SH or —(CH₂)₀₋₃Z, where Z represents —H, —OY³, —SY³, halogen, NHY³, —CO—NY¹Y² or —CO—OY³, where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
R⁶ and R⁷ independently of one another represent H, cyano, (optionally fluorinated) C₁₋₁₀-alkyl, (optionally fluorinated) C₂₋₁₀-alkenyl, (optionally fluorinated) C₂₋₁₀-alkynyl, hydroxy, NO₂, NH₂, COOH or halogen (in particular F, Cl, Br),
R⁸ represents (optionally fluorinated) C₁₋₁₀-alkyl, (optionally fluorinated) C₂₋₁₀-alkenyl, (optionally fluorinated) C₂₋₁₀-alkynyl, (optionally fluorinated) C₄₋₁₀-cycloalkyl or —(CH₂)₀₋₂—(HZ²), where HZ² represents a 4- to 7-membered heterocycle having up to two heteroatoms selected from the group consisting of N, O and S, where each of these groups may be substituted by —OH, CO₂H or NH₂;
R⁹ represents H, F, CH₃, CF₃, CH₂F or CHF₂;
where -MOD represents —(NR¹⁰)ₙ-(G1)ₒ-G2-G3, where
R¹⁰ represents H or C₁-C₃-alkyl;
G1 represents —NHCO— or —CONH— (where, if G1 represents —NHCO—, R¹⁰ does not represent NH₂);
n represents 0 or 1;
o represents 0 or 1; and
G2 represents a straight-chain and/or branched hydrocarbon group which has 1 to 10 carbon atoms and which may be interrupted once or more than once by one or more of the groups —O—, —S—, —SO—, SO₂, -NRy-, -NRyCO—, CONRy-, -NRyNRy-, —SO₂NRyNRy-, -CONRyNRy- (where R^{y} represents H, phenyl, C1-C10-alkyl, C2-C10-alkenyl or C2-C10-alkynyl, each of which may be substituted by NHCONH₂, —COOH, —OH, —NH₂, NH—CNNH₂, sulphonamide, sulphone, sulphoxide or sulphonic acid), —CO—, or —CR^{x}═N—O— (where Rx represents H, C1-C3-alkyl or phenyl), where the hydrocarbon chain including any side chains may be substituted by —NHCONH₂, —COOH, —OH, —NH₂, NH—CNNH₂, sulphonamide, sulphone, sulphoxide or sulphonic acid, G3 represents —H or —COOH, and where the group -MOD preferably has at least one group —COOH;
and the salts, solvates and salts of the solvates and epimers thereof.

In the case of binder conjugates of the KSP inhibitors of the formula (II), at most one representative of R¹, R³ and R⁴ (alternatively to one of the conditions given above) may represent -L-BINDER, where L represents a linker and BINDER represents an antibody, where the antibody may optionally be attached to a plurality of active compound molecules.

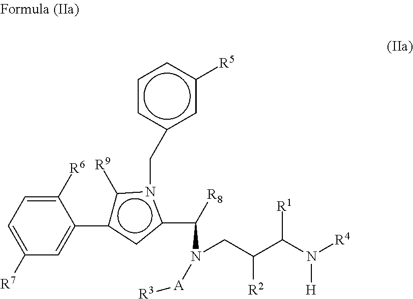

where
R¹ represents -L-BINDER, H or —(CH₂)₀₋₃Z, where Z represents —H, —NHY³, —OY³, —SY³, halogen, —CO—NY¹Y² or —CO—OY³,
where Y¹ and Y² independently of one another represent H, NH₂, —(CH₂CH₂O)₀₋₃—(CH₂)₀₋₃Z′ or —CH(CH₂W)Z′, and Y³ represents H or —(CH₂)₀₋₃Z′, where Z′ represents H, NH₂, SO₃H, COOH, —NH—CO—CH₂—CH₂—CH(NH₂)COOH or —(CO—NH—CHY⁴)₁₋₃COOH; where W represents H or OH; where Y⁴ represents straight-chain or branched C₁₋₆ alkyl which is optionally substituted by —NHCONH₂, or represents aryl or benzyl which are optionally substituted by —NH₂;
R² and R⁴ independently of one another represent H, -SG_{lys}-(CO)₀₋₁—R⁴, —CO—CHY⁴—NHY⁵ or —(CH₂)₀₋₃Z, or R² and R⁴ together (with formation of a pyrrolidine ring) represent —CH₂—CHR¹¹— or —CHR¹¹—CH₂—, or R² represents H, —CO—CHY⁴—NHY⁵ or —(CH₂)₀₋₃Z and R⁴ represents -L-#1, where R¹¹ represents H, NH₂, SO₃H, COOH, SH, halogen (in particular F or Cl), C₁₋₄-alkyl, C₁₋₄-haloalkyl, C₁₋₄-alkoxy, hydroxyl-substituted C₁₋₄-alkyl, COO(C₁₋₄-alkyl) or OH;
where SG_{lys} represents a group which can be cleaved by a lysosomal enzyme, in particular a group consisting of a di- or tripeptide, R⁴ represents a C₁₋₁₀-alkyl, C₅₋₁₀-aryl or C₆₋₁₀-aralkyl, C₅₋₁₀-heteroalkyl, C₁₋₁₀-alkyl-O—C₆₋₁₀-aryl, C₅₋₁₀-heterocycloalkyl, heteroaryl, heteroarylalkyl, heteroarylalkoxy, C₁₋₁₀-alkoxy, C₆₋₁₀-aryloxy or C₆₋₁₀-aralkoxy, C₅₋₁₀-heteroaralkoxy, C₁₋₁₀-alkyl-O—C₆₋₁₀-aryloxy, C₅₋₁₀-heterocycloalkoxy group which may be mono- or polysubstituted by —NH₂, —NH-alkyl, —N(alkyl)₂, NH—CO-alkyl, N(alkyl)-COalkyl, -SG₃H, —SO₂NH₂, —SO₂—N(alkyl)₂, —COOH, —CONH₂, —CON(alkyl)₂ or —OH, —H or a group —Oₓ—(CH₂CH₂O)_{y}—R^{4″} (where x represents 0 or 1 and v represents a number from 1 to 10 and R⁴ represents —H, -alkyl (preferably C₁₋₁₂-alkyl), —CH₂—COOH, —CH₂—CH₂—COOH or —CH₂—CH₂—NH₂);
where Z represents —H, halogen, —OY³, —SY³, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where Y⁴ represents straight-chain or branched C₁₋₆-alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl;
A represents CO, SO, SO₂, SO₂NH or CNNH₂;
R³ represents -L-BINDER or an optionally substituted alkyl, aryl, heteroaryl, heteroalkyl, heterocycloalkyl group, preferably -L-BINDER or a C₁₋₁₀-alkyl, C₆₋₁₀-aryl or C₆₋₁₀-aralkyl, C₅₋₁₀-heteroalkyl, C₁₋₁₀-alkyl-O—C₆₋₁₀-aryl or C₅₋₁₀-heterocycloalkyl group which may be substituted by 1-3 —OH groups, 1-3 halogen atoms, 1-3 halogenated alkyl groups (each having 1-3 halogen atoms), 1-3 O-alkyl groups, 1-3 —SH groups, 1-3 —S-alkyl groups, 1-3 —O—CO-alkyl groups, 1-3 —O—CO—NH-alkyl groups, 1-3 —NH—CO-alkyl groups, 1-3 —NH—CO—NH-alkyl groups, 1-3 —S(O)ₙ-alkyl groups, 1-3 —SO₂—NH-alkyl groups, 1-3 —NH-alkyl groups, 1-3 —N(alkyl)₂ groups, 1-3 —NH₂ groups or 1-3 —(CH₂)₀₋₃Z groups, where Z represents —H, halogen, —OY³, —SY³, —NHY³, —CO—NY¹Y² or —CO—OY³, where Y¹ and Y² independently of one another represent H, NH₂ or —(CH₂)₀₋₃Z′ and Y³ represents H, —(CH₂)₀₋₃—CH(NHCOCH₃)Z′, —(CH₂)₀₋₃—CH(NH₂)Z′ or —(CH₂)₀₋₃Z′, where Z′ represents H, SO₃H, NH₂ or COOH
(where “alkyl” preferably represents C₁₋₁₀-alkyl);
R⁵ represents H, F, NH₂, NO₂, halogen, SH or —(CH₂)₀₋₃Z, where Z represents —H, halogen, —OY³, —SY³, —NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where L represents a linker and BINDER represents a binder or a derivative thereof, where the binder may optionally be attached to a plurality of active compound molecules,
R⁶ and R⁷ independently of one another represent H, cyano, (optionally fluorinated) C₁₋₁₀-alkyl, (optionally fluorinated) C₂₋₁₀-alkenyl, (optionally fluorinated) C₂₋₁₀-alkynyl, hydroxy or halogen,
R⁸ represents (optionally fluorinated) C₁₋₁₀-alkyl, (optionally fluorinated) C₄₋₁₀-cycloalkyl or optionally substituted oxetane; and
R⁹ represents H, F, CH₃, CF₃, CH₂F or CHF₂;
and the salts, solvates, salts of the solvates and epimers thereof.

Preference according to the invention is furthermore given to the KSP inhibitor/antibody conjugates below:

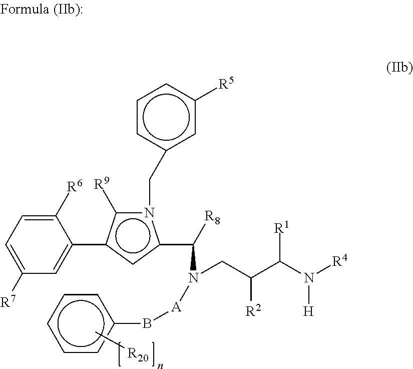

where R¹, R², R⁴, R⁵, R⁶, R⁷, R⁸ and R⁹ have the same meaning as in formula (II) or (IIa), A represents CO, B represents a single bond, —O—CH₂— or —CH₂—O— and R²⁰ represents NH₂, F, CF₃ or CH₃, and n represents 0, 1 or 2.

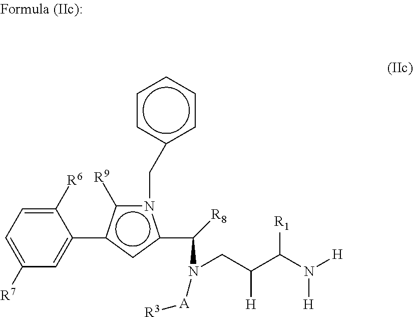

where A, R¹, R³, R⁶, R⁷, R⁸ and R⁹ have the same meaning as in formula (II) or (IIa), where A preferably represents CO and R³ represents —CH₂OH, —CH₂OCH₃, CH(CH₃)OH or CH(CH₃)OCH₃.

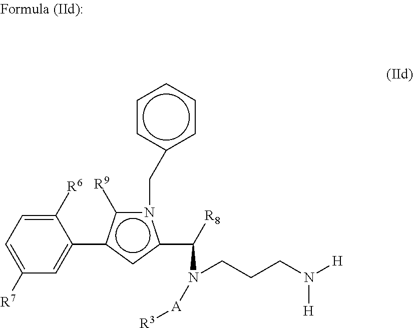

where A, R³, R⁶, R⁷, R⁸ and R⁹ have the same meaning as in formula (II) or (IIa), where A preferably represents CO and R³ represents —CH₂—Sₓ—(CH₂)₀₋₄—CHY⁵—COOH, where x represents 0 or 1 and Y⁵ represents H or NHY⁶, where Y⁶ represents H or —COCH₃.

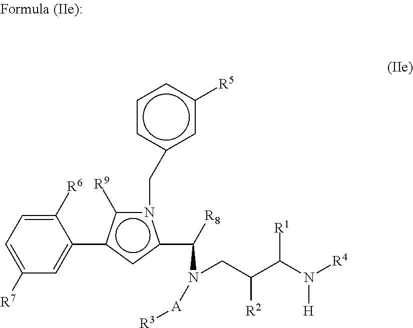

where A, R², R³, R⁴, R⁶, R⁷, R⁸ and R⁹ have the same meaning as in formula (II) or (IIa) and R¹ represents -L-BINDER.

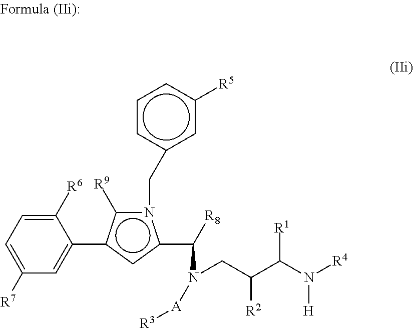

where A, R¹, R², R³, R⁶, R⁷, R⁸ and R⁹ have the same meaning as in formula (II) or (IIa) and R⁴ represents -L-BINDER, preferably an enzymatically cleavable binder, so that after cleavage R⁴=H. R¹ or R³ particularly preferably represent -MOD.

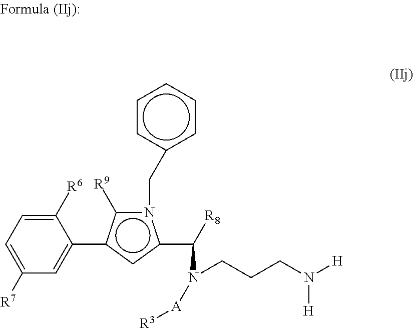

where
R³ represents -L-#1;
A represents CO; and
R⁶, R⁷, R⁸ and R⁹ have the same meaning as in formula (I)

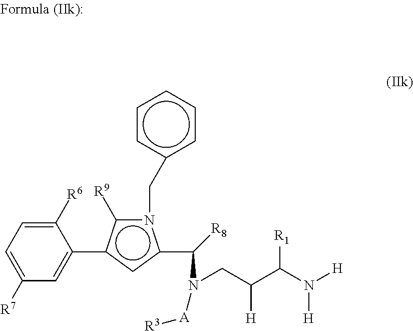

where
R¹ represents -L-#1;
A represents CO and R³ represents —CH₂OH;
R³, R⁶, R⁷, R⁸ and R⁹ have the same meaning as in formula (I).

Furthermore, it is preferred when in the compounds of the formulae (II), (IIa), (IIb), (IIe), (IId), (IIe), (IIi), (IIj) and (IIk) (alone or in combination):

Z represents Cl or Br;
R¹ represents —(CH₂)₀₋₃Z, where Z represents —COOH or —CO—NY¹Y², where Y² represents —(CH₂CH₂O)₀₋₃—(CH₂)₀₋₃Z′ and Y¹ represents H, NH₂ or —(CH₂CH₂O)₀₋₃—(CH₂)₀₋₃Z′;
Y¹ represents H, Y² represents —(CH₂CH₂O)₃—CH₂CH₂Z′ and Z′ represents —COOH;
Y¹ represents H, Y² represents —CH₂CH₂Z′ and Z′ represents —(CONHCHY⁴)₂COOH;
Y¹ represents H, Y² represents —CH₂CH₂Z′, Z′ represents —(CONHCHY⁴)₂COOH and one of the Y⁴ radicals represents z-propyl and the other —(CH₂)₃—NHCONH₂;
Y¹ represents H, Y² represents —CH₂CH₂Z′, Z′ represents —(CONHCHY⁴)₂COOH and one of the Y⁴ radicals represents —CH₃ and the other —(CH₂)₃—NHCONH₂;
Y⁴ represents straight-chain or branched C₁₋₆-alkyl which is optionally substituted by —NHCONH₂; at least one Y⁴ representative is selected from the group consisting of z-propyl and —CH₃;
Y¹ represents H, Y² represents —CH₂CH₂Z′, Z′ represents —CONHCHY⁴COOH and Y⁴ represents aryl or benzyl which are optionally substituted by —NH₂;
Y⁴ represents aminobenzyl;
R² represents —(CH₂)₀₋₃Z and Z represents —SY³;
R⁴ represents —CO—CHY⁴—NHY⁵ and Y⁵ represents H;
R⁴ represents —CO—CHY⁴—NHY⁵ and Y⁵ represents —CO—CHY⁶—NH₂; or
Y⁴ represents straight-chain or branched C₁₋₆-alkyl which is optionally substituted by —NHCONH₂.

Furthermore, it is preferred when in the formula (I) or (II) R¹, R² or R³ represents -MOD. Particularly preferably, R³ represents -MOD and R¹ or R⁴ represents -L-#1 or -L-BINDER,

where -MOD represents —(NR¹⁰)ₙ-(G1)ₒ-G2-G3, where
R¹⁰ represents H or C₁-C₃-alkyl;
G1 represents —NHCO— or —CONH— (where, if G1 represents —NHCO—, R¹⁰ does not represent NH₂);
n represents 0 or 1;
o represents 0 or 1; and
G2 represents a straight-chain and/or branched hydrocarbon group which has 1 to 10 carbon atoms and which may be interrupted once or more than once by one or more of the groups —O—, —S—, —SO—, SO₂, -NRy-, -NRyCO—, CONRy-, -NRyNRy-, —SO₂NRyNRy-, -CONRyNRy- (where R^{y} represents H, phenyl, C1-C10-alkyl, C2-C10-alkenyl or C2-C10-alkynyl, each of which may be substituted by NHCONH₂, —COOH, —OH, —NH₂, —NH—CNNH₂, sulphonamide, sulphone, sulphoxide or sulphonic acid), —CO—, or —CR^{x}═N—O— (where Rx represents H, C1-C3-alkyl or phenyl), where the hydrocarbon chain including any side chains may be substituted by NHCONH₂, —COOH, —OH, —NH₂, NH—CNNH₂, sulphonamide, sulphone, sulphoxide or sulphonic acid, where G3 represents —H or —COOH, and where the group -MOD preferably has at least one group —COOH;

Particularly preferably, the group -MOD has a (preferably terminal) —COOH group, for example in a betaine group. Preferably, the group -MOD has the formula —CH₂—Sₓ—(CH₂)₀₋₄—CHY⁵—COOH where x is 0 or 1, and Y⁵ represents H or NHY⁶, where Y⁶ represents H or —COCH₃.

Other particularly preferred compounds have the formula (III) below:

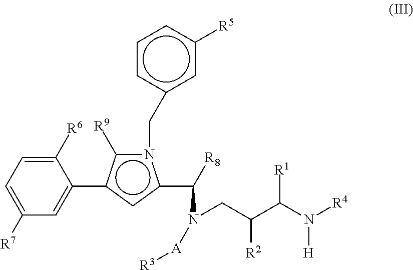

where
R¹ represents -L-BINDER, H or —(CH₂)₀₋₃Z, where Z represents —H, —NHY³, —OY³, —SY³, halogen, —CO—NY¹Y² or —CO—OY³,
where Y¹ and Y² independently of one another represent H, NH₂, —(CH₂CH₂O)₀₋₃—(CH₂)₀₋₃Z′ or —CH(CH₂W)Z′, and Y³ represents H or —(CH₂)₀₋₃Z′, where Z′ represents H, NH₂, SO₃H, COOH, —NH—CO—CH₂—CH₂—CH(NH₂)COOH or —(CO—NH—CHY⁴)₁₋₃COOH;
where Y⁴ represents straight-chain or branched C₁₋₆ alkyl which is optionally substituted by —NHCONH₂, or represents aryl or benzyl which are optionally substituted by —NH₂;
R² and R⁴ independently of one another represent H, -SG_{lys}-(CO)₀₋₁—R^{4′}, —CO—CHY⁴—NHY⁵ or —(CH₂)₀₋₃Z,
or R² and R⁴ together (with formation of a pyrrolidine ring) represent —CH₂—CHR¹¹— or —CHR¹¹—CH₂—, where R¹¹ represents H, NH₂, SO₃H, COOH, SH, halogen (in particular F or Cl), C₁₋₄-alkyl, C₁₋₄haloalkyl, C₁₋₄-alkoxy, hydroxyl-substituted C₁₋₄-alkyl, COO(C₁₋₄alkyl) or OH;
where SG_{lys} represents a group which can be cleaved by a lysosomal enzyme, in particular a group consisting of a di- or tripeptide, R^{4′} represents a C₁₋₁₀-alkyl, C₅₋₁₀-aryl or C₆₋₁₀-aralkyl, C₅₋₁₀-heteroalkyl, C₁₋₁₀-alkyl-O—C₆₋₁₀-aryl, C₅₋₁₀-heterocycloalkyl, heteroaryl, heteroarylalkyl, heteroarylalkoxy, C₁₋₁₀-alkoxy, C₆₋₁₀-aryloxy or C₆₋₁₀-aralkoxy, C₅₋₁₀-heteroaralkoxy, C₁₋₁₀-alkyl-O—C₆₋₁₀-aryloxy, C₅₋₁₀-heterocycloalkoxy group which may be mono- or polysubstituted by —NH₂, —NH-alkyl, —N(alkyl)₂, NH—CO-alkyl, N(alkyl)-COalkyl, —SO₃H, —SO₂NH₂, —SO₂—N(alkyl)₂, —COOH, —CONH₂, —CON(alkyl)₂ or —OH, —H or a group —Oₓ—(CH₂CH₂O)_{y}—R^{4″} (where x represents 0 or 1 and v represents a number from 1 to 10 and R^{4″} represents —H, -alkyl (preferably C₁₋₁₂-alkyl), —CH₂—COOH, —CH₂—CH₂—COOH or —CH₂—CH₂—NH₂); where Z represents —H, halogen, —OY³, —SY³, NHY³, —CO—NY¹Y² or —CO—OY³, where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where Y⁴ represents straight-chain or branched C₁₋₆-alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl;
A represents CO, SO, SO₂, SO₂NH or CNNH₂;
R³ represents -L-BINDER or an optionally substituted alkyl, aryl, heteroaryl, heteroalkyl, heterocycloalkyl group, or —CH₂—Sₓ—(CH₂)₀₋₄—CHY⁵—COOH, where x represents 0 or 1 and Y⁵ represents H or NHY⁶, where Y⁶ represents H or —COCH₃, preferably -L-BINDER or a C₁₋₁₀-alkyl, C₆₋₁₀-aryl or C₆₋₁₀-aralkyl, C₅₋₁₀-heteroalkyl, C₁₋₁₀-alkyl-O—C₆₋₁₀-aryl or C₅₋₁₀-heterocycloalkyl group which may be substituted by 1-3 —OH groups, 1-3 halogen atoms, 1-3 halogenated alkyl groups (each having 1-3 halogen atoms), 1-3 O-alkyl groups, 1-3 —SH groups, 1-3 —S-alkyl groups, 1-3 —O—CO-alkyl groups, 1-3 —O—CO—NH-alkyl groups, 1-3 —NH—CO-alkyl groups, 1-3 —NH—CO—NH-alkyl groups, 1-3 —S(O)ₙ-alkyl groups, 1-3 —SO₂—NH-alkyl groups, 1-3 —NH-alkyl groups, 1-3 —N(alkyl)₂ groups, 1-3 —NH₂ groups or 1-3 —(CH₂)₀₋₃Z groups, where Z represents —H, halogen, —OY³, —SY³, —NHY³, —CO—NY¹Y² or —CO—OY³, where Y¹ and Y² independently of one another represent H, NH₂ or —(CH₂)₀₋₃Z′ and Y³ represents H, —(CH₂)₀₋₃—CH(NHCOCH₃)Z′, —(CH₂)₀₋₃—CH(NH₂)Z′ or —(CH₂)₀₋₃Z′, where Z′ represents H, SO₃H, NH₂ or COOH,
(where “alkyl” preferably represents C₁₋₁₀-alkyl);
R⁵ represents H, F, NH₂, NO₂, halogen, SH or —(CH₂)₀₋₃Z, where Z represents —H, halogen, —OY³, —SY³, —NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where L represents a linker and BINDER represents the antibody, where the binder may optionally be attached to a plurality of active compound molecules,
R⁶ and R⁷ independently of one another represent H, cyano, (optionally fluorinated) C₁₋₁₀-alkyl, (optionally fluorinated) C₂₋₁₀-alkenyl, (optionally fluorinated) C₂₋₁₀-alkynyl, hydroxy or halogen,
R⁸ represents (optionally fluorinated) C₁₋₁₀-alkyl, (optionally fluorinated) C₄₋₁₀-cycloalkyl or optionally substituted oxetane; and
R⁹ represents H, F, CH₃, CF₃, CH₂F or CHF₂;
and the salts, solvates, salts of the solvates and epimers thereof.

Furthermore, it is preferred when (alone or in combination) in the formula (I), (Ia), (II), (IIa), (IIb), (IIe), (IId), (IIe), (IIi), (IIj), (IIk) or (III):

Z represents Cl or Br;
R¹ represents —(CH₂)₀₋₃Z, where Z represents —CO—NY¹Y², where Y² represents —(CH₂CH₂O)₀₋₃—(CH₂)₀₋₃Z′ and Y¹ represents H, NH₂ or —(CH₂CH₂O)₀₋₃—(CH₂)₀₋₃Z′;
Y¹ represents H, Y² represents —(CH₂CH₂O)₃—CH₂CH₂Z′ and Z′ represents —COOH;
Y¹ represents H, Y² represents —CH₂CH₂Z′ and Z′ represents —(CONHCHY⁴)₂COOH;
Y¹ represents H, Y² represents —CH₂CH₂Z′, Z′ represents —(CONHCHY⁴)₂COOH and one Y⁴ representative represents z-propyl and the other represents —(CH₂)₃—NHCONH₂;
Y¹ represents H, Y² represents —CH₂CH₂Z′, Z′ represents —(CONHCHY⁴)₂COOH and one Y⁴ representative represents —CH₃ and the other represents —(CH₂)₃—NHCONH₂;
Y⁴ represents straight-chain or branched C₁₋₆-alkyl which is optionally substituted by —NHCONH₂; at least one Y⁴ representative is selected from the group consisting of i-propyl and —CH₃;
Y¹ represents H, Y² represents —CH₂CH₂Z′, Z′ represents —CONHCHY⁴COOH and Y⁴ represents aryl or benzyl which are optionally substituted by —NH₂;
Y⁴ represents aminobenzyl;
R² represents —(CH₂)₀₋₃Z and Z represents —SY³;
R⁴ represents —CO—CHY⁴—NHY⁵ and Y⁵ represents H;
R⁴ represents —CO—CHY⁴—NHY⁵ and Y⁵ represents —CO—CHY⁶—NH₂; and/or
Y⁴ represents straight-chain or branched C₁₋₆-alkyl which is optionally substituted by —NHCONH₂.

Preference is furthermore given to compounds of the formula (I), (Ia), (II), (IIa) or (III) where

R¹ represents H, -L-#1 or -L-BINDER, -MOD or —(CH₂)₀₋₃Z, where Z represents —H, —NHY³, —OY³, —SY³, halogen, —CO—NY¹Y² or —CO—OY³,
where Y¹ and Y² independently of one another represent H, NH₂, —(CH2CH₂O)₀₋₃—(CH₂)₀₋₃Z′ (e.g. —(CH₂)₀₋₃Z′) or —CH(CH₂W)Z′, and Y³ represents H or —(CH₂)₀₋₃Z′, where Z′ represents H, NH₂, SO₃H, COOH, —NH—CO—CH₂—CH₂—CH(NH₂)COOH or —(CO—NH—CHY⁴)₁₋₃COOH, where W represents H or OH,
where Y⁴ represents straight-chain or branched C₁₋₆ alkyl which is optionally substituted by —NHCONH₂, or represents aryl or benzyl which are optionally substituted by —NH₂;
R² represents H, —CO—CHY⁴—NHY⁵ or —(CH₂)₀₋₃Z,
where Z represents —H, halogen, —OY³, —SY³, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where Y⁴ independently of one another represents straight-chain or branched C₁₋₆-alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl;
R⁴ represents H or -L-#1 or -L-BINDER (where -L-#1 or -L-BINDER is an enzymatically cleavable linker leading to the conversion of R⁴ into H);
A represents CO, SO, SO₂, SO₂NH or CNNH₂;
R³ represents -L-#1 or -L-BINDER, -MOD or an optionally substituted alkyl, cycloalkyl, aryl, heteroaryl, heteroalkyl, heterocycloalkyl group, preferably a C₁₋₁₀-alkyl, C₆₋₁₀-aryl or C₆₋₁₀-aralkyl, C₅₋₁₀-heteroalkyl, C₁₋₁₀-alkyl-O—C₆₋₁₀-aryl or C₅₋₁₀-heterocycloalkyl group which may be substituted by 1-3 —OH groups, 1-3 halogen atoms, 1-3 halogenated alkyl groups (each having 1-3 halogen atoms), 1-3 O-alkyl groups, 1-3 —SH groups, 1-3 —S-alkyl groups, 1-3 —O—CO-alkyl groups, 1-3 —O—CO—NH-alkyl groups, 1-3 —NH—CO-alkyl groups, 1-3 —NH—CO—NH-alkyl groups, 1-3 —S(O)ₙ-alkyl groups, 1-3 —SO₂—NH-alkyl groups, 1-3 —NH-alkyl groups, 1-3 —N(alkyl)₂ groups, 1-3 —NH((CH₂CH₂O)1-20H) groups, 1-3 —NH₂ groups or 1-3 —(CH₂)₀₋₃Z groups, where Z represents —H, halogen, —OY³, —SY³, —NHY³, —CO—NY¹Y² or —CO—OY³, where Y¹ and Y² independently of one another represent H, NH₂ or —(CH₂)₀₋₃Z′ and Y³ represents H, —(CH₂)₀₋₃—CH(NHCOCH₃)Z′, —(CH₂)₀₋₃—CH(NH₂)Z′ or —(CH₂)₀₋₃Z′, where Z′ represents H, SO₃H, NH₂ or COOH (where “alkyl” is preferably C₁₋₁₀-alkyl);
R⁵ represents H, -MOD, NH₂, NO₂, halogen (in particular F, Cl, Br), —CN, CF₃, —OCF₃, —CH₂F, —CH₂F, SH or —(CH₂)₀₋₃Z, where Z represents —H, —OY³, —SY³, halogen, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
R⁶ and R⁷ independently of one another represent H, cyano, (optionally fluorinated) C₁₋₁₀-alkyl, (optionally fluorinated) C₂₋₁₀-alkenyl, (optionally fluorinated) C₂₋₁₀-alkynyl, hydroxy, NO₂, NH₂, COOH or halogen (in particular F, Cl, Br),
R⁸ represents (optionally fluorinated) C₁₋₁₀-alkyl, (optionally fluorinated) C₂₋₁₀-alkenyl, (optionally fluorinated) C₂₋₁₀-alkynyl or (optionally fluorinated) C₄₋₁₀-cycloalkyl;
where one of the substituents R¹ and R³ represents -L-#1 or -L-BINDER,
L represents the linker and #1 represents the bond to the antibody and BINDER represents the antibody,
R⁹ represents H, F, CH₃, CF₃, CH₂F or CHF₂;
where -MOD represents —(NR¹⁰)ₙ-(G1)ₒ-G2-G3, where
R¹⁰ represents H or C₁-C₃-alkyl;
G1 represents —NHCO— or —CONH— (where, if G1 represents —NHCO—, R¹⁰ does not represent NH₂);
n represents 0 or 1;
o represents 0 or 1; and
G2 represents a straight-chain and/or branched hydrocarbon group which has 1 to 10 carbon atoms and which may be interrupted once or more than once by one or more of the groups —O—, —S—, —SO—, SO₂, -NRy-, -NRyCO—, CONRy-, -NRyNRy-, —SO₂NRyNRy-, -CONRyNRy- (where R^{y} represents H, phenyl, C1-C10-alkyl, C2-C10-alkenyl or C2-C10-alkynyl, each of which may be substituted by NHCONH₂, —COOH, —OH, —NH₂, NH—CNNH₂, sulphonamide, sulphone, sulphoxide or sulphonic acid), —CO—, —CR^{x}═N—O— (where Rx represents H, C1-C3-alkyl or phenyl), where the hydrocarbon chain including any side chains may be substituted by —NHCONH₂, —COOH, —OH, —NH₂, NH—CNNH₂, sulphonamide, sulphone, sulphoxide or sulphonic acid, G3 represents —H or —COOH, and where the group -MOD preferably has at least one group —COOH;
and the salts, solvates, salts of the solvates and epimers thereof.

Preference is furthermore given to compounds of the formula (I), (Ia), (II), (IIa) or (III) in which

R¹ represents H, -L-#1 or -L-BINDER, -MOD or —(CH₂)₀₋₃Z, where Z represents —H, —NHY³, —OY³, —SY³, halogen, —CO—NY¹Y² or —CO—OY³,
where Y¹ and Y² independently of one another represent H, NH₂, —(CH2CH₂O)₀₋₃—(CH₂)₀₋₃Z′ (e.g. —(CH₂)₀₋₃Z′) or —CH(CH₂W)Z′, and Y³ represents H or —(CH₂)₀₋₃Z′, where Z′ represents H, NH₂, SO₃H, COOH, —NH—CO—CH₂—CH₂—CH(NH₂)COOH or —(CO—NH—CHY⁴)₁₋₃COOH, where W represents H or OH,
where Y⁴ represents straight-chain or branched C₁₋₆ alkyl which is optionally substituted by —NHCONH₂, or represents aryl or benzyl which are optionally substituted by —NH₂;
R² represents H, —CO—CHY⁴—NHY⁵ or —(CH₂)₀₋₃Z,
where Z represents —H, halogen, —OY³, —SY³, NHY³, —CO—NY¹Y² or —CO—OY³, where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
where Y⁴ represents straight-chain or branched C₁₋₆-alkyl which is optionally substituted by —NHCONH₂, or represents aryl or benzyl which are optionally substituted by —NH₂, and Y⁵ represents H or —CO—CHY⁶—NH₂, where Y⁶ represents straight-chain or branched C₁₋₆-alkyl;
R⁴ represents H,
A represents CO, SO, SO₂, SO₂NH or CNNH₂;
R³ represents -L-#1 or -L-BINDER, -MOD or an optionally substituted alkyl, cycloalkyl, aryl, heteroaryl, heteroalkyl, heterocycloalkyl group, preferably a C₁₋₁₀-alkyl, C₆₋₁₀-aryl or C₆₋₁₀-aralkyl, C₅₋₁₀-heteroalkyl, C₁₋₁₀-alkyl-O—C₆₋₁₀-aryl or C₅₋₁₀-heterocycloalkyl group which may be substituted by 1-3 —OH groups, 1-3 halogen atoms, 1-3 halogenated alkyl groups (each having 1-3 halogen atoms), 1-3 O-alkyl groups, 1-3 —SH groups, 1-3 —S-alkyl groups, 1-3 —O—CO-alkyl groups, 1-3 —O—CO—NH-alkyl groups, 1-3 —NH—CO-alkyl groups, 1-3 —NH—CO—NH-alkyl groups, 1-3 —S(O)ₙ-alkyl groups, 1-3 —SO₂—NH-alkyl groups, 1-3 —NH-alkyl groups, 1-3 —N(alkyl)₂ groups, 1-3 —NH((CH₂CH₂O) 1-20H) groups, 1-3 —NH₂ groups or 1-3 —(CH₂)₀₋₃Z groups, where Z represents —H, halogen, —OY³, —SY³, —NHY³, —CO—NY¹Y² or —CO—OY³, where Y¹ and Y² independently of one another represent H, NH₂ or —(CH₂)₀₋₃Z′ and Y³ represents H, —(CH₂)₀₋₃—CH(NHCOCH₃)Z′, —(CH₂)₀₋₃—CH(NH₂)Z′ or —(CH₂)₀₋₃Z′, where Z′ represents H, SO₃H, NH₂ or COOH (where “alkyl” is preferably C₁₋₁₀-alkyl);
R⁵ represents H, -MOD, NH₂, NO₂, halogen (in particular F, Cl, Br), —CN, CF₃, —OCF₃, —CH₂F, —CH₂F, SH or —(CH₂)₀₋₃Z, where Z represents —H, —OY³, —SY³, halogen, NHY³, —CO—NY¹Y² or —CO—OY³,
where Y¹ and Y² independently of one another represent H, NH₂ or —(CH₂)₀₋₃Z′, and Y³ represents H or —(CH₂)₀₋₃Z′, where Z′ represents H, SO₃H, NH₂ or COOH;
R⁶ and R⁷ independently of one another represent H or halogen (in particular F, Cl, Br);
R⁸ represents (optionally fluorinated) C₁₋₁₀-alkyl;
where one of the substituents R¹ and R³ represents -L-#1 or -L-BINDER,
L represents the linker and #1 represents the bond to the antibody and BINDER represents the antibody,
R⁹ represents H, F, CH₃, CF₃, CH₂F or CHF₂;
where -MOD represents —CH₂—Sₓ—(CH₂)₀₋₄—CHY⁵—COOH where x is 0 or 1, and Y⁵ represents H or NHY⁶, where Y⁶ represents H or —COCH₃,
   and the salts, solvates, salts of the solvates and epimers thereof.

Preference is furthermore given to the following compounds which may optionally be present together with an acid such as, for example, trifluoroacetic acid. These compounds may be attached via the positions corresponding to the positions R¹, R³ and R⁴ via a linker to the antibody (where a hydrogen atom is substituted by the linker):
N-(3-Aminopropyl)-N-{(1R)-1-[1-benzyl-4-(2,5-difluorophenyl)-1H-pyrrol-2-yl]-2,2-dimethylpropyl}-2-hydroxyacetamide;
(2S)-2-amino-4-[{(1R)-1-[1-benzyl-4-(2,5-difluorophenyl)-1H-pyrrol-2-yl]-2,2-dimethylpropyl}(glycoloyl)amino]-N-methylbutanamide (1:1);
N-(3-aminopropyl)-N-{(1S)-1-[1-benzyl-4-(2,5-difluorophenyl)-1H-pyrrol-2yl]-2,2-dimethylpropyl}acetamide;
N-(3-aminopropyl)-N-{(1S)-1-[1-benzyl-4-(2,5-difluorophenyl)-1H-pyrrol-2-yl]-2,2-dimethylpropyl}-2-hydroxyacetamide;
S-(1-{2-[(N-{(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}-beta-alanyl)amino]ethyl}-2,5-dioxopyrrolidin-3-yl)-L-cysteine;
S-(1-{2-[(N-{(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}-beta-alanyl)amino]ethyl}-2,5-dioxopyrrolidin-3-yl)-L-cysteine;
S-[1-(2-{[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)-2,5-dioxopyrrolidin-3-yl]-L-cysteine;
N-[19-(3(R/S)-{[(2R)-2-amino-2-carboxyethyl]sulphanyl}-2,5-dioxopyrrolidin-1-yl)-17-oxo-4,7,10,13-tetraoxa-16-azanonadecan-1-oyl]-R/S-{2-[(3-aminopropyl){(1R)-1-[1-benzyl-4-(2,5-difluorophenyl)-1H-pyrrol-2-yl]-2,2-dimethylpropyl}amino]-2-oxoethyl}homocysteine;
S-{(3R/S)-1-[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]-2,5-dioxopyrrolidin-3-yl}-L-cysteine;
S-[(3R/S)-1-(2-{[6-({2-[(3-aminopropyl){(1R)-1-[1-benzyl-4-(2,5-difluorophenyl)-1H-pyrrol-2-yl]-2,2-dimethylpropyl}amino]-2-oxoethyl}sulphanyl)hexanoyl]amino}ethyl)-2,5-dioxopyrrolidin-3-yl]-L-cysteine;
S-{1-[2-({[(1R,3S)-3-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)cyclopentyl]carbonyl}amino)ethyl]-2,5-dioxopyrrolidin-3-yl}-L-cysteine;
S-(2-{[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)-L-cysteine;
N⁶—(N-{(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}-beta-alanyl)-N²—{N-[6-(3-{[(2R)-2-amino-2-carboxyethyl]sulphanyl}-2,5-dioxopyrrolidin-1-yl)hexanoyl]-L-valyl-L-alanyl}-L-lysine;
N-[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]-L-glutamine;
N⁶—(N-{(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}-beta-alanyl)-L-lysine;
N-(3-aminopropyl)-N-{(1R)-1-[1-benzyl-4-(2,5-difluorophenyl)-1H-pyrrol-2-yl]-2,2-dimethylpropyl}-3,3,3-trifluoropropanamide;
N-(3-aminopropyl)-N-{(1R)-1-[1-benzyl-4-(2,5-difluorophenyl)-1H-pyrrol-2-yl]-2,2-dimethylpropyl}-4-fluorobenzamide;
N-(3-aminopropyl)-N-{(1R)-1-[1-benzyl-4-(2,5-difluorophenyl)-1H-pyrrol-2-yl]-2,2-dimethylpropyl}acetamide;
N-(3-aminopropyl)-N-{(1R)-1-[1-benzyl-4-(2,5-difluorophenyl)-1H-pyrrol-2-yl]-2,2-dimethylpropyl}-4-(trifluoromethyl)benzamide;
(2S)-2-amino-4-[{(1R)-1-[1-benzyl-4-(2,5-difluorophenyl)-1H-pyrrol-2-yl]-2,2-dimethylpropyl}(glycoloyl)amino]butanoic acid;
(2S)-2-amino-N-(2-aminoethyl)-4-[{(1R)-1-[1-benzyl-4-(2,5-difluorophenyl)-1H-pyrrol-2-yl]-2,2-dimethylpropyl}(glycoloyl)amino]butan amide;
4-[(2-{[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)amino]-3-{[(2R)-2-amino-2-carboxyethyl]sulphanyl}-4-oxobutanoic acid;
4-[(2-{[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)amino]-2-{[(2R)-2-amino-2-carboxyethyl]sulphanyl}-4-oxobutanoic acid;
N-{(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}-beta-alanine;
N-{(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}-L-serine;
N-{(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}-L-alanine;
N-{(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}glycine;
N-(3-aminopropyl)-N-{(1R)-1-[1-benzyl-4-(2,5-difluorophenyl)-1H-pyrrol-2-yl]-2,2-dimethylpropyl}-4-methylbenzamide;
N-(3-aminopropyl)-N-{(1R)-1-[1-benzyl-4-(2,5-difluorophenyl)-1H-pyrrol-2-yl]-2,2-dimethylpropyl}-4-(methylsulphanyl)benzamide;
(2S)—N-(3-aminopropyl)-N-{(1R)-1-[1-benzyl-4-(2,5-difluorophenyl)-1H-pyrrol-2-yl]-2,2-dimethylpropyl}-2-hydroxypropanamide;
N-(3-aminopropyl)-N-{(1R)-1-[1-benzyl-4-(2,5-difluorophenyl)-1H-pyrrol-2-yl]-2,2-dimethylpropyl}-2-(methylsulphanyl)acetamide;
(2S)—N-(3-aminopropyl)-N-{(1R)-1-[4-benzyl-1-(2,5-difluorophenyl)-1H-pyrazol-3-yl]-2,2-dimethylpropyl}-2-hydroxypropanamide;
methyl 4-[(3-aminopropyl){(1R)-1-[1-benzyl-4-(2,5-difluorophenyl)-1H-pyrrol-2-yl]-2,2-dimethylpropyl}amino]-4-oxobutanoate;
4-[(3-aminopropyl) {(1R)-1-[1-benzyl-4-(2,5-difluorophenyl)-1H-pyrrol-2-yl]-2,2-dimethylpropyl}amino]-4-oxobutanoic acid;
(2R)-22-[(3R/S)-3-{[(2R)-2-amino-2-carboxyethyl]sulphanyl}-2,5-dioxopyrrolidin-1-yl]-2-[({2-[(3-aminopropyl) {(1R)-1-[1-benzyl-4-(2,5-difluorophenyl)-1H-pyrrol-2-yl]-2,2-dimethylpropyl}amino]-2-oxoethyl}sulphanyl)methyl]-4,20-dioxo-7,10,13,16-tetraoxa-3,19-diazadocosan-1-oic acid;
N-acetyl-S-{2-[(3-aminopropyl) {(1R)-1-[1-benzyl-4-(2,5-difluorophenyl)-1H-pyrrol-2-yl]-2,2-dimethylpropyl}amino]-2-oxoethyl}-L-cysteine;
N-acetyl-S-[2-([3-(L-alanylamino)propyl]{(1R)-1-[1-benzyl-4-(2,5-difluorophenyl)-1H-pyrrol-2-yl]-2,2-dimethylpropyl}amino)-2-oxoethyl]-L-cysteine;
(2S)—N-(3-aminopropyl)-N-{(1R)-1-[1-benzyl-4-(2,5-difluorophenyl)-1H-pyrrol-2-yl]-2,2-dimethylpropyl}tetrahydrofuran-2-carboxamide;
3-({2-[(3-aminopropyl){(1R)-1-[1-benzyl-4-(2,5-difluorophenyl)-1H-pyrrol-2-yl]-2,2-dimethylpropyl}amino]-2-oxoethyl}sulphanyl)propanoic acid;
S-{2-[(3-aminopropyl){(1R)-1-[1-benzyl-4-(2,5-difluorophenyl)-1H-pyrrol-2-yl]-2,2-dimethylpropyl}amino]-2-oxoethyl}homocysteine;
4-amino-N-(3-aminopropyl)-N-{(1R)-1-[1-benzyl-4-(2,5-difluorophenyl)-1H-pyrrol-2-yl]-2,2-dimethylpropyl}benzamide;
4-[(2-{[(2R)-2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)-2-carboxyethyl]amino}-2-oxoethyl)amino]-3-{[(2R)-2-amino-2-carboxyethyl]sulphanyl}-4-oxobutanoic acid;
4-[(2-{[(2R)-2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)-2-carboxyethyl]amino}-2-oxoethyl)amino]-2-{[(2R)-2-amino-2-carboxyethyl]sulphanyl}-4-oxobutanoic acid.

Particular preference according to the invention is given to the following compounds of the formula IV where R¹, R², R³, R⁴ and R⁵ have the meanings mentioned above (as mentioned, for example for formula (I) or (II)):

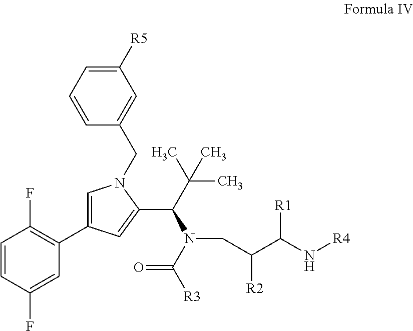

Particular preference is given to the compounds of the formula IV where R¹ and R⁵ represent H or -L-#1; R² and R⁴ represent H or R² and R⁴ together (with formation of a pyrrolidine ring) represent —CH₂—CHR¹¹— or —CHR¹¹—CH₂—, where R¹¹ represents H; and R³ represents CH₂OH, CH(CH₃)OH or -L-#1, where one of the substituents R¹ and R³ represents -L-#1. In addition, particular preference is given to the compounds of the formula IV where R¹ represents H or COOH; R² and R⁵ represent H; R⁴ represents -L-#1; and R³ represents CH₂OH or CH(CH₃)OH, where -L-#1 is an enzymatically cleavable linker leading to the conversion of R⁴ into H.

### Linkers

The literature discloses various options for covalently coupling (conjugating) organic molecules to binders such as, for example antibodies (see, for example, K. Lang and J. W. Chin. Chem. Rev. 2014, 114, 4764-4806, M. Rashidian et al. Bioconjugate Chem. 2013, 24, 1277-1294). Preference according to the invention is given to conjugation of the KSP inhibitors to an antibody via one or more sulphur atoms of cysteine residues of the antibody which are either already present as free thiols or generated by reduction of disulphide bridges, and/or via one or more NH groups of lysine residues of the antibody. However, it is also possible to attach the KSP inhibitor to the antibody via tyrosine residues, via glutamine residues, via residues of unnatural amino acids, via free carboxyl groups or via sugar residues of the antibody. For coupling, use is made of linkers. Linkers can be categorized into the group of the linkers which can be cleaved in vivo and the group of the linkers which are stable in vivo (see L. Ducry and B. Stump, Bioconjugate Chem. 21, 5-13 (2010)). The linkers which can be cleaved in vivo have a group which can be cleaved in vivo, where, in turn, a distinction may be made between groups which are chemically cleavable in vivo and groups which are enzymatically cleavable in vivo. “Chemically cleavable in vivo” and “enzymatically cleavable in vivo” means that the linkers or groups are stable in circulation and are cleaved only at or in the target cell by the chemically or enzymatically different environment therein (lower pH; elevated glutathione concentration; presence of lysosomal enzymes such as proteases, or glyosidases such as, for example, β-glucuronidases), thus releasing the low-molecular weight KSP inhibitor or a derivative thereof. Groups which can be cleaved chemically in vivo are in particular disulphide, hydrazone, acetal and aminal; groups which can be cleaved enzymatically in vivo, in particular those which are cleavable by lysosomal enzymes, are in particular the 2-8-oligopeptide group, especially a tri- or dipeptide group or glycoside. Peptide cleaving sites are disclosed in Bioconjugate Chem. 2002, 13, 855-869 and Bioorganic & Medicinal Chemistry Letters 8 (1998) 3341-3346 and also Bioconjugate Chem. 1998, 9, 618-626. These include, for example, valine-alanine, valine-lysine, valine-citrulline, alanine-lysine and phenylalanine-lysine (optionally with additional amide group).

Linkers which are stable in vivo are distinguished by a high stability (less than 5% metabolites after 24 hours in plasma) and do not have the chemically or enzymatically in vivo cleavable groups mentioned above.

The linker -L- preferably has one of the basic structures (i) to (iv) below:

### (i) —(CO)ₘ—SG1-L1-L2-

### (ii) —(CO)ₘ-L1-SG-L1-L2-

(iii) —(CO)ₘ-L1-L2-

### (iv) —(CO)ₘ-L1-SG-L2

where m is 0 or 1; SG is a (chemically or enzymatically) in vivo cleavable group (in particular disulphide, hydrazone, acetal and aminal; or a 2-8-oligopeptide group which can be cleaved by a protease), SG1 is an oligopeptide group or preferably a dipeptide group, L1 independently of one another represent in vivo stable organic groups, and L2 represents a coupling group to the binder or a single bond. Here, coupling is preferably to a cysteine residue or a lysine residue of the antibody. Alternatively, coupling can be to a tyrosine residue, glutamine residue or to an unnatural amino acid of the antibody. The unnatural amino acids may contain, for example, aldehyde or keto groups (such as, for example, formylglycine) or azide or alkyne groups (see Lan & Chin, Cellular Incorporation of Unnatural Amino Acids and Bioorthogonal Labeling of Proteins, Chem. Rev. 2014, 114, 4764-4806).

Particular preference according to the invention is given to the basic linker structure (iii). Via metabolization, the administration of a conjugate according to the invention having a basic linker structure (iii) and coupling of the linker to a cysteine or lysine residue of the antibody leads to cysteine or lysine derivatives of the formulae below:

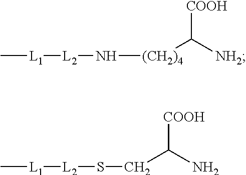

where L1 is in each case attached to the low-molecular weight KSP inhibitor, for example a compound of the formula (I), (Ia), (II), (IIa), (IIb), (IIca), (IId), (IIe), (IIf), (III) or (IV).

Preference according to the invention is also given to the basic linker structures (ii) and (iv), in particular when attachment is at position R¹, in particular when group L1 has one of the following structures:

(a) —NH—(CH₂)₀₋₄—(CHCH₃)₀₋₄—CHY⁵—CO—Y⁷, where Y⁵ represents H or NHY⁶, where Y⁶ represents H or —COCH₃, and Y⁷ represents a single bond or —NH—(CH₂)₀₋₄ —CHNH₂—CO—, so that after cleavage the corresponding structure —NH—(CH₂)₀₋₄—(CHCH₃)₀₋₄—CHY⁵—COOH or —NH—(CH₂)₀₋₄—(CHCH₃)₀₋₄—CHY⁵—CO—NH—(CH₂)₀₋₄—CHNH₂—COOH is obtained.
(b) —CH₂—Sₓ—(CH₂)₀₋₄—CHY⁵—CO—, where x is 0 or 1, and Y⁵ represents H or NHY⁶, where Y⁶ represents H or —COCH₃, such that after cleavage the corresponding structure —CH₂—Sₓ—(CH₂)₀₋₄—CHY⁵—COOH is obtained.

Preference according to the invention is also given to the basic linker structure (i) when attached to position R⁴, in particular if m=0.

If the linker is attached to a cysteine side chain or a cysteine residue, L2 is preferably derived from a group which reacts with the sulphhydryl group of the cysteine. These include haloacetyls, maleimides, aziridines, acryloyls, arylating compounds, vinylsulphones, pyridyl disulphides, TNB thiols and disulphide-reducing agents. These groups generally react in an electrophilic manner with the sulphhydryl bond, forming a sulphide (e.g. thioether) or disulphide bridge. Preference is given to stable sulphide bridges. L2 is preferably

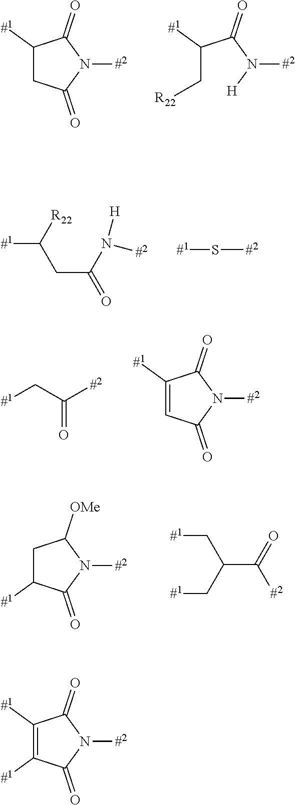

where
#¹ denotes the point of attachment to the sulphur atom of the antibody,
#² denotes the point of attachment to group L¹, and
R²² represents COOH, COOR, COR, CONHR, CONR₂ (where R in each case represents C1-3-alkyl), CONH₂, preferably COOH.

Particularly preferred for L2 is:

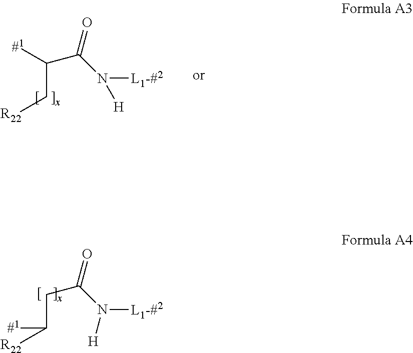

where #¹ denotes the point of attachment to the sulphur atom of the antibody, #² denotes the point of attachment to the active compound, x represents 1 or 2, and R²² represents COOH, COOR, COR, CONR₂, CONHR (where R in each case represents C1-3-alkyl), CONH₂, preferably COOH. It is preferred when x=1 and R²² represents COOH.

In a conjugate according to the invention or in a mixture of the conjugates according to the invention, the bonds to a cysteine residue of the antibody are present, to an extent of preferably more than 80%, particularly preferably more than 90% (in each case based on the total number of bonds of the linker to the antibody), particularly preferably as one of the two structures of the formula A3 or A4. Here, the structures of the formula A3 or A4 are generally present together, preferably in a ratio of from 60:40 to 40:60, based on the number of bonds to the antibody. The remaining bonds are then present as the structure

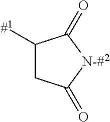

According to the invention, L1 is preferably represented by the formula

#¹—(NR¹⁰)ₙ-(G1)ₒ-G2-#²

where
R¹⁰ represents H, NH₂ or C₁-C₃-alkyl;
G1 represents —NHCO—, —CONH— or

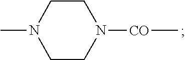

(R¹⁰ is preferably not NH₂, if G1 represents NHCO or

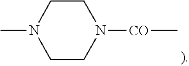

n represents 0 or 1;
o represents 0 or 1; and
G2 represents a straight-chain or branched hydrocarbon chain which has 1 to 100 carbon atoms from arylene groups and/or straight-chain and/or branched and/or cyclic alkylene groups and which may be interrupted once or more than once by one or more of the groups —O—, —S—, —SO—, SO₂, -NRy-, -NRyCO—, —C(NH)NRy-, CONRy-, -NRyNRy-, —SO₂NRyNRy-, -CONRyNRy- (where R^{y} represents H, phenyl, C1-C10-alkyl, C2-C10-alkenyl or C2-C10-alkynyl, each of which may be substituted by NHCONH₂, —COOH, —OH, —NH₂, NH—CNNH₂, sulphonamide, sulphone, sulphoxide or sulphonic acid), —CO—, —CR^{x}═N—O— (where R^{x} represents H, C1-C3-alkyl or phenyl) and/or a 3- to 10-membered aromatic or non-aromatic heterocycle having up to 4 heteroatoms selected from the group consisting of N, O and S, —SO— or —SO₂— (preferably

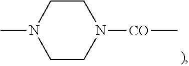

where the hydrocarbon chain including any side chains may be substituted by —NHCONH₂, —COOH, —OH, —NH₂, NH—CNNH₂, sulphonamide, sulphone, sulphoxide or sulphonic acid.
G2 represents a straight-chain or branched hydrocarbon chain having 1 to 100 carbon atoms from arylene groups and/or straight-chain and/or branched and/or cyclic alkylene groups and which may be interrupted once or more than once by one or more of the groups —O—, —S—, —SO—, SO₂, —NH—, —CO—, —NHCO—, —CONH—, —NMe-, —NHNH—, —SO₂NHNH—, —CONHNH— and a 5- to 10-membered aromatic or non-aromatic heterocycle having up to 4 heteroatoms selected from the group consisting of N, O and S, or —SO— (preferably

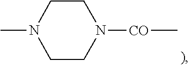

where the side chains, if present, may be substituted by —NHCONH₂, —COOH, —OH, —NH₂, NH—CNNH₂, sulphonamide, sulphone, sulphoxide or sulphonic acid.
G2 preferably represents a straight-chain or branched hydrocarbon chain having 1 to 100 carbon atoms from arylene groups and/or straight-chain and/or branched and/or cyclic alkylene groups and which may be interrupted once or more than once by one or more of the groups —O—, —S—, —SO—, SO₂, —NH—, —CO—, —NHCO—, —CONH—, —NMe-, —NHNH—, —SO₂NHNH—, —CONHNH—, —CR^{x}═N—O— (where R^{x} represents H, C1-C3-alkyl or phenyl) and a 3- to 10-membered, for example 5- to 10-membered, aromatic or non-aromatic heterocycle having up to 4 heteroatoms selected from the group consisting of N, O and S, —SO— or —SO₂— (preferably

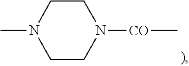

where the hydrocarbon chain including the side chains, if present, may be substituted by —NHCONH₂, —COOH, —OH, —NH₂, NH—CNNH₂, sulphonamide, sulphone, sulphoxide or sulphonic acid.

Further interrupting groups in G2 are preferably

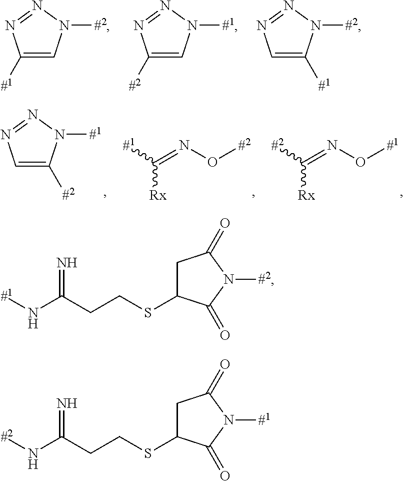

where R^{x} represents H, C₁-C₃-alkyl or phenyl.

Here, #¹ is the bond to the KSP inhibitor and #² is the bond to the coupling group to the antibody (e.g. L2).

A straight-chain or branched hydrocarbon chain of arylene groups and/or straight-chain and/or branched and/or cyclic alkylene groups generally comprises a α,ω-divalent alkyl radical having the respective number of carbon atoms stated. The following may be mentioned by way of example and as preferred: methylene, ethane-1,2-diyl (1,2-ethylene), propane-1,3-diyl (1,3-propylene), butane-1,4-diyl (1,4-butylene), pentane-1,5-diyl (1,5-pentylene), hexane-1,6-diyl (1,6-hexylene), heptane-1,7-diyl (1,7-hexylene), octane-1,8-diyl (1,8-octylene), nonane-1,9-diyl (1,9-nonylene), decane-1,10-diyl (1,10-decylene). However, the alkylene groups in the hydrocarbon chain may also be branched, i.e. one or more hydrogen atoms of the straight-chain alkylene groups mentioned above may optionally be substituted by C1-10-alkyl groups, thus forming side chains. The hydrocarbon chain may furthermore contain cyclic alkylene groups (cycloalkanediyl), for example 1,4-cyclohexanediyl or 1,3-cyclopentanediyl. These cyclic groups may be unsaturated. In particular, aromatic groups (arylene groups), for example phenylene, may be present in the hydrocarbon group. In turn, in the cyclic alkylene groups and the arylene groups, too, one or more hydrogen atoms may optionally be substituted by C1-10-alkyl groups. In this way, an optionally branched hydrocarbon chain is formed. This hydrocarbon chain has a total of 0 to 100 carbon atoms, preferably 1 to 50, particularly preferably 2 to 25 carbon atoms.

The side chains, if present, may be mono- or polysubstituted, identically or differently, by —NHCONH₂, —COOH, —OH, —NH₂, NH—CNNH₂, sulphonamide, sulphone, sulphoxide or sulphonic acid.

The hydrocarbon chain may be interrupted once or more than once, identically or differently, by —O—, —S—, —SO—, SO₂, —NH—, —CO—, —NHCO—, —CONH—, —NMe-, —NHNH—, —SO₂NHNH—, —CONHNH— and a 5- to 10-membered aromatic or non-aromatic heterocycle having up to 4 heteroatoms selected from the group consisting of N, O and S, —SO— or —SO₂— (preferably

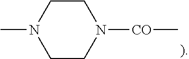

Further interrupting groups in G2 are preferably

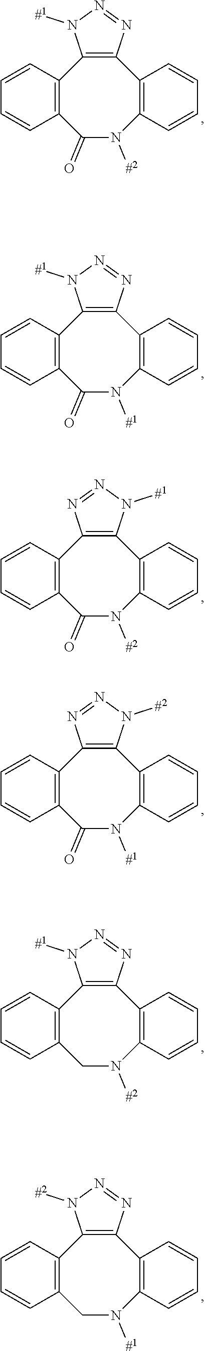

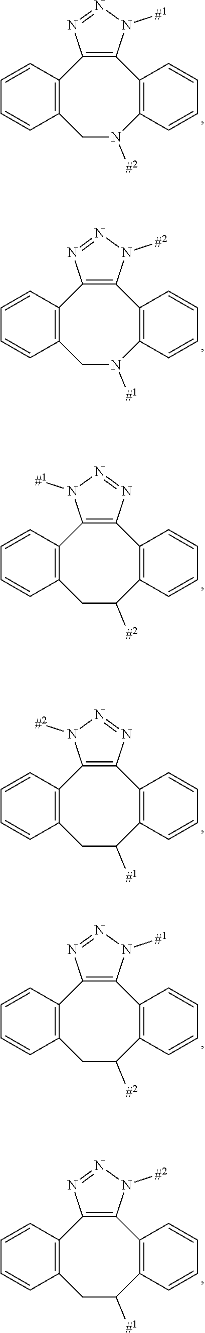

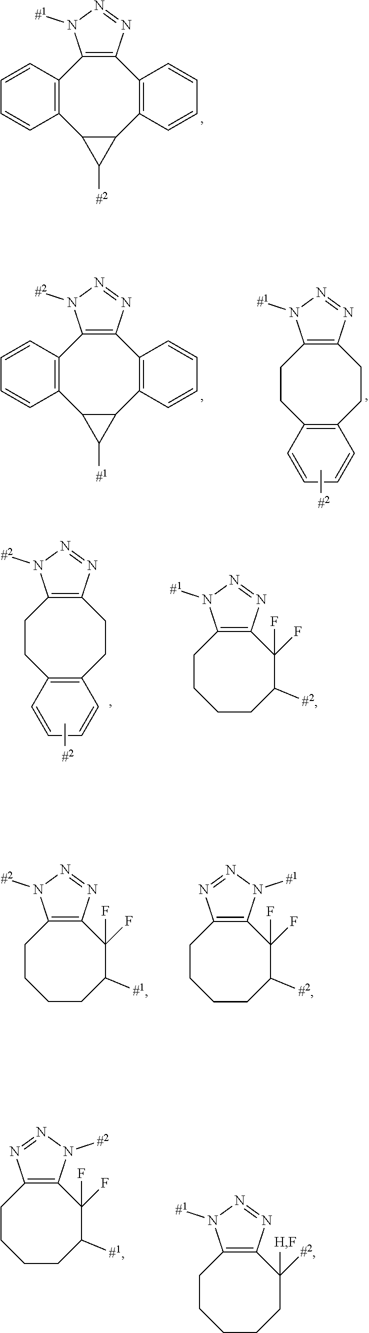

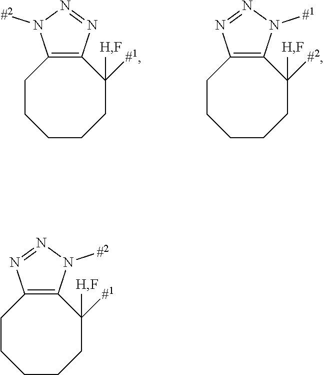

Preferably, the linker corresponds to the formula below:

§—(CO)m-L1-L2-§§

where
m represents 0 or 1;
§ represents the bond to the active compound molecule and
§§ represents the bond to the binder peptide or protein, and
L1 and L2 have the meaning given above.

Particularly preferably, L1 has the formula —NR11B—, where

R¹¹ represents H or NH₂;
B represents —[(CH₂)ₓ—(X⁴)_{y}]_{w}—(CH₂)_{z}—,
w=0 to 20;
x=0 to 5;
y=0 or 1;
z=0 to 5; and
X⁴ represents —O—, —CONH—, —NHCO— or

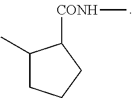

Linkers L which are preferred in accordance with the invention have the formula below:

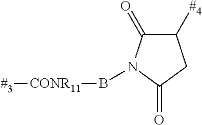

where
#3 represents the bond to the active compound molecule,
#4 represents the bond to the binder peptide or protein,
R11 represents H or NH₂;
B represents —[(CH₂)ₓ—(X⁴)_{y}]w-(CH₂)_{z}—,
w=0 to 20;
x=0 to 5;
y=0 or 1;
z=1 to 5; and
X⁴ represents —O—, —CONH—, —NHCO— or

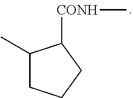

The linkers mentioned above are especially preferred in conjugates of the formula (I) or (II) in which the linker couples by substitution of a hydrogen atom at R1 or in combination with a cleavable linker SG1 at R4, i.e. R1 represents -L-#1 or R4 represents -SG1-L-#1, where #1 represents the bond to the antibody.

Preference in accordance with the invention is furthermore given to the linkers below: In a conjugate according to the invention or in a mixture of the conjugates according to the invention, the bonds to a cysteine residue of the antibody are present, to an extent of preferably more than 80%, particularly preferably more than 90% (in each case based on the total number of bonds of the linker to the antibody), particularly preferably as one of the two structures of the formula A5 or A6:

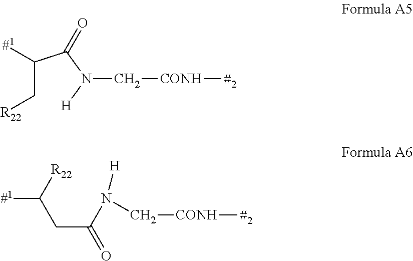

where
#₁ denotes the point of attachment to the sulphur atom of the antibody,
#² denotes the point of attachment to group L¹, and
   R²² represents COOH, COOR, COR, CONR₂, CONHR (where R in each case represents C1-3-alkyl), CONH₂, preferably COOH.

Here, the structures of the formula A5 or A6 are generally present together, preferably in a ratio of from 60:40 to 40:60, based on the number of bonds to the antibody. The remaining bonds are then present as the structure

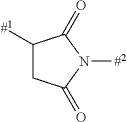

Other linkers -L- attached to a cysteine side chain or cysteine residue have the formula below:

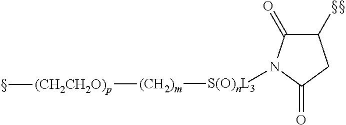

where
§ represents the bond to the active compound molecule and
§§ represents the bond to the binder peptide or protein,
m represents 0, 1, 2 or 3;
n represents 0, 1 or 2;
p represents 0 to 20; and
L3 represents

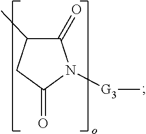

where
o represents 0 or 1;
and
G3 represents a straight-chain or branched hydrocarbon chain having 1 to 100 carbon atoms from arylene groups and/or straight-chain and/or cyclic alkylene groups and which may be interrupted once or more than once by one or more of the groups —O—, —S—, —SO—, SO₂, —NH—, —CO—, —NHCO—, —CONH—, —NMe-, —NHNH—, —SO₂NHNH—, —CONHNH— and a 3- to 10-membered (preferably 5- to 10-membered) aromatic or non-aromatic heterocycle having up to 4 heteroatoms selected from the group consisting of N, O and S, —SO— or SO₂ (preferably

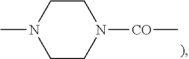

where the side chains, if present, may be substituted by —NHCONH₂, —COOH, —OH, —NH₂, NH—CNNH₂, sulphonamide, sulphone, sulphoxide or sulphonic acid.

In the formula above, preferably

m represents 1;
p represents 0;
n represents 0;
and L3 represents

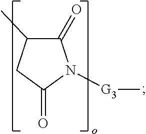

where
o represents 0 or 1; and
G3 represents —(CH₂CH₂O)ₛ(CH₂)ₜ(CONH)ᵤ CH₂CH₂O)ᵥ(CH₂)_{w}—, where
s, t, v and w each independently of one another are from 0 to 20 and u is 0 or 1.

Preferred groups L1 in the formula §—(CO)m-L1-L2-§§ above are those below, where r in each case independently of one another represents a number from 0 to 20, preferably from 0 to 15, particularly preferably from 1 to 20, especially preferably from 2 to 10:

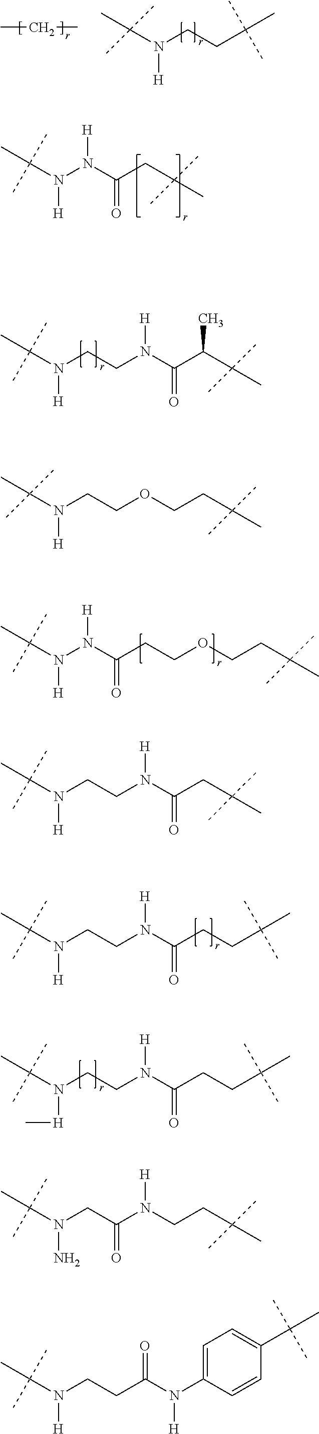

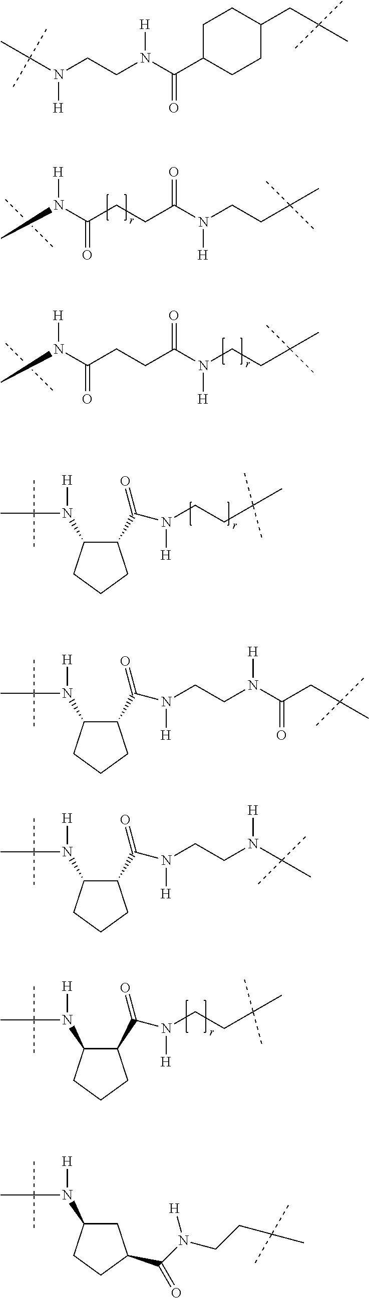

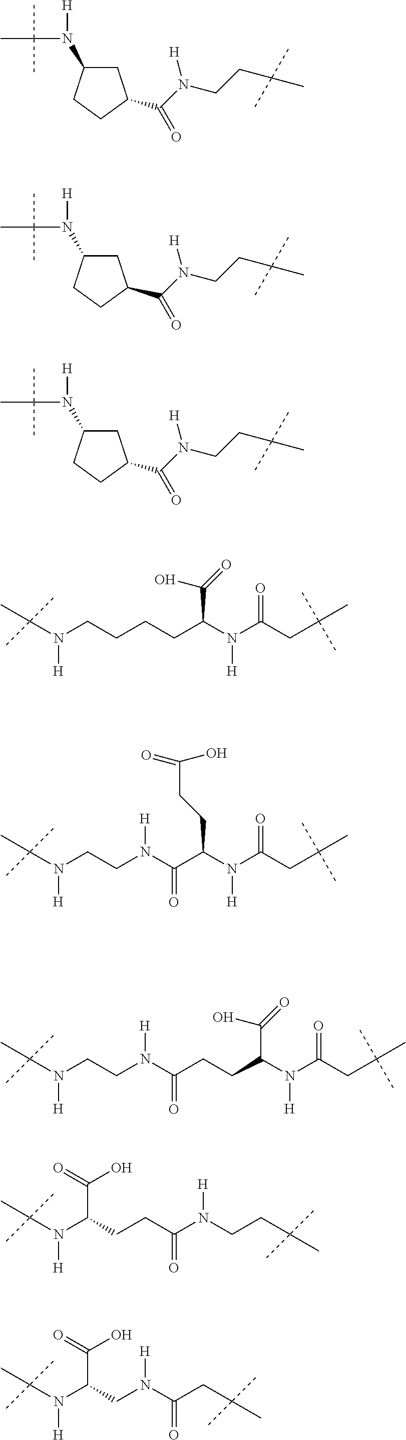

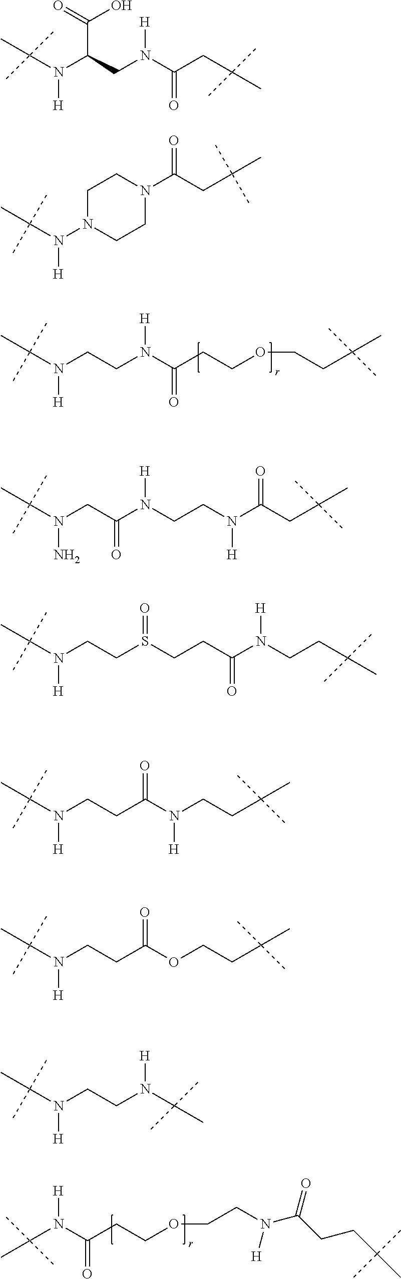

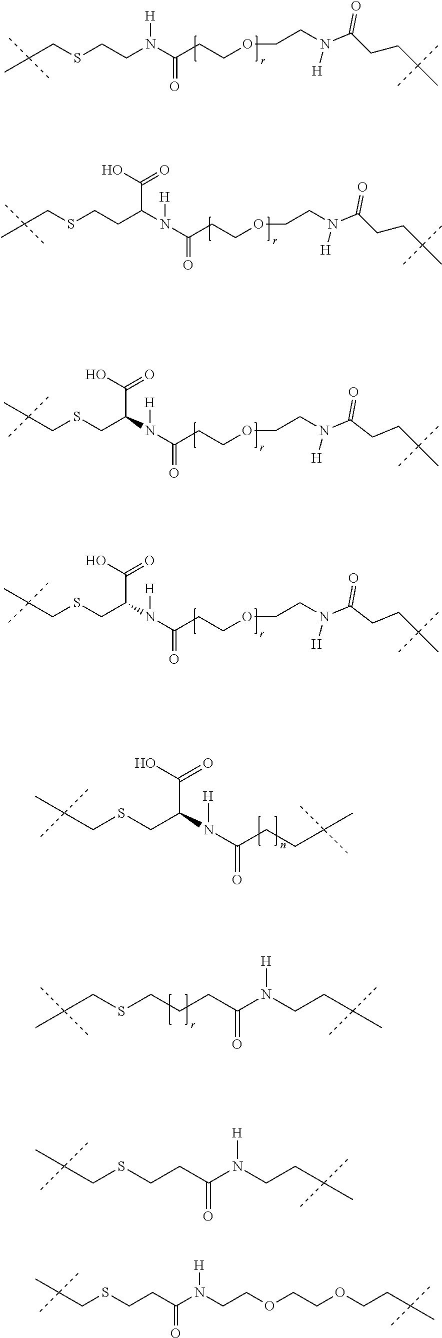

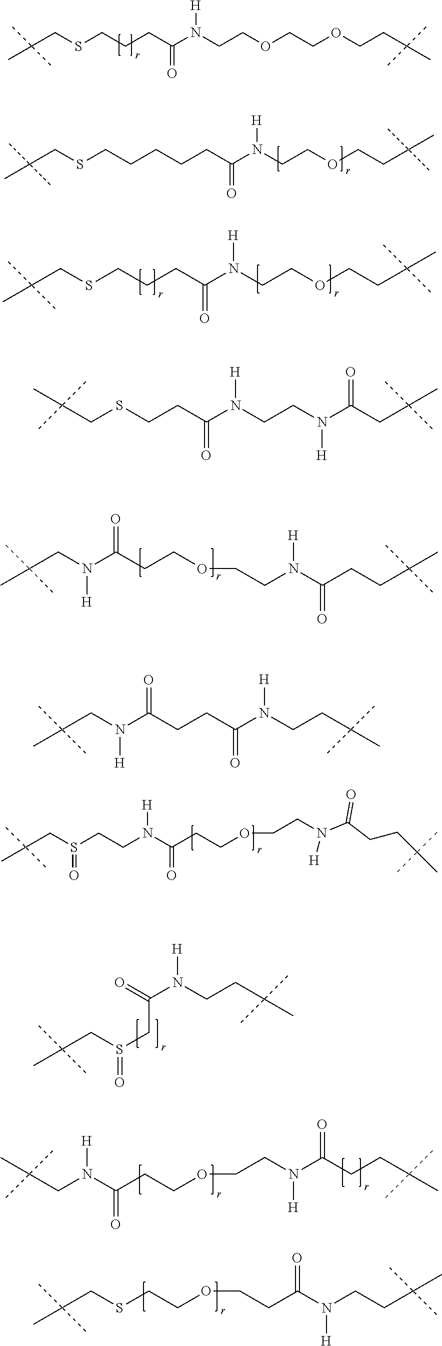

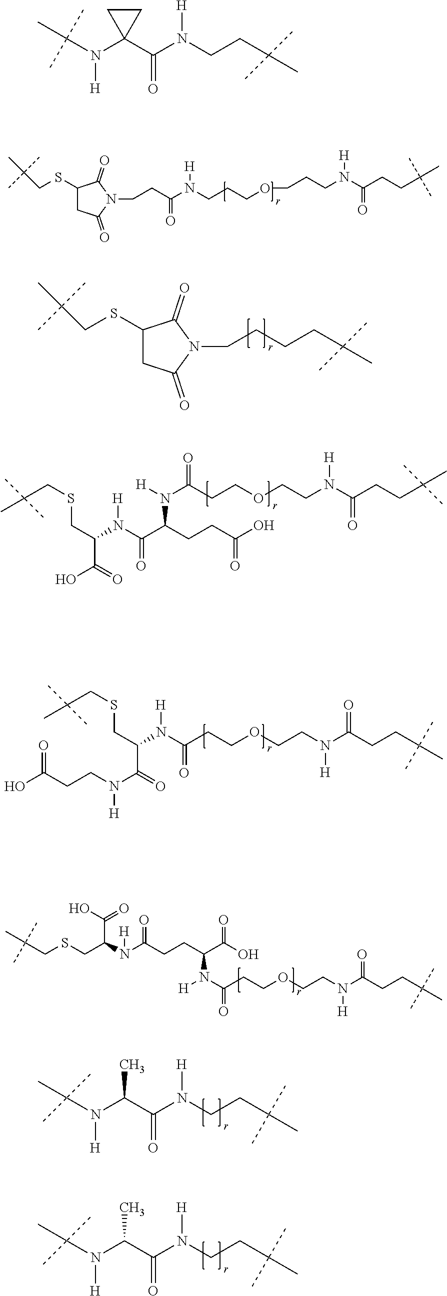

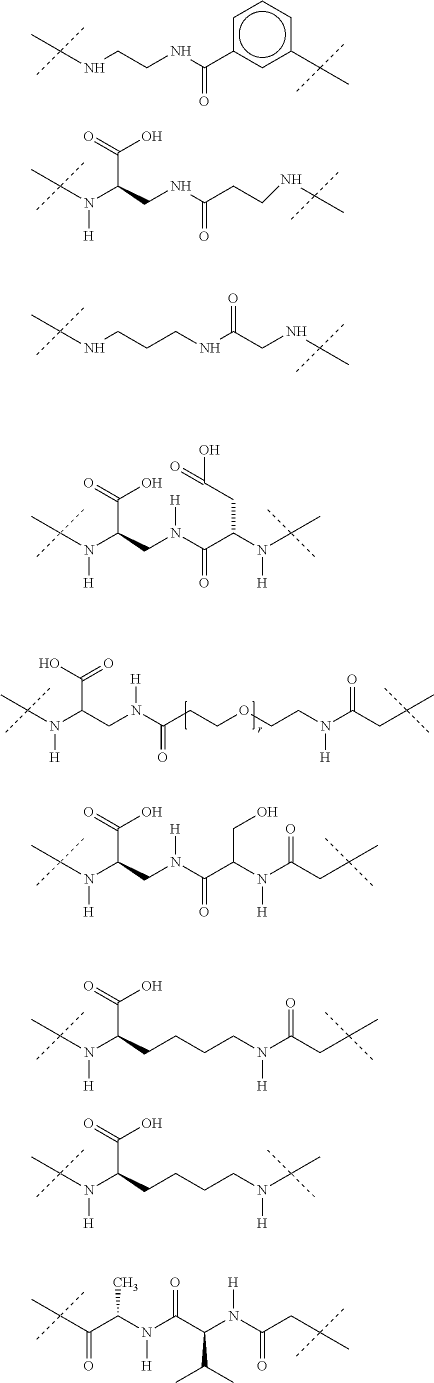

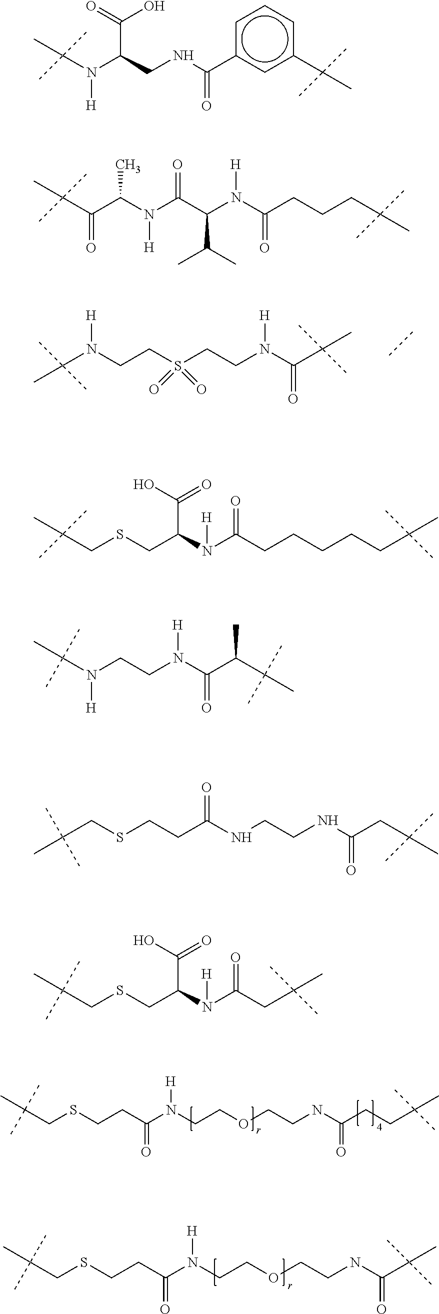

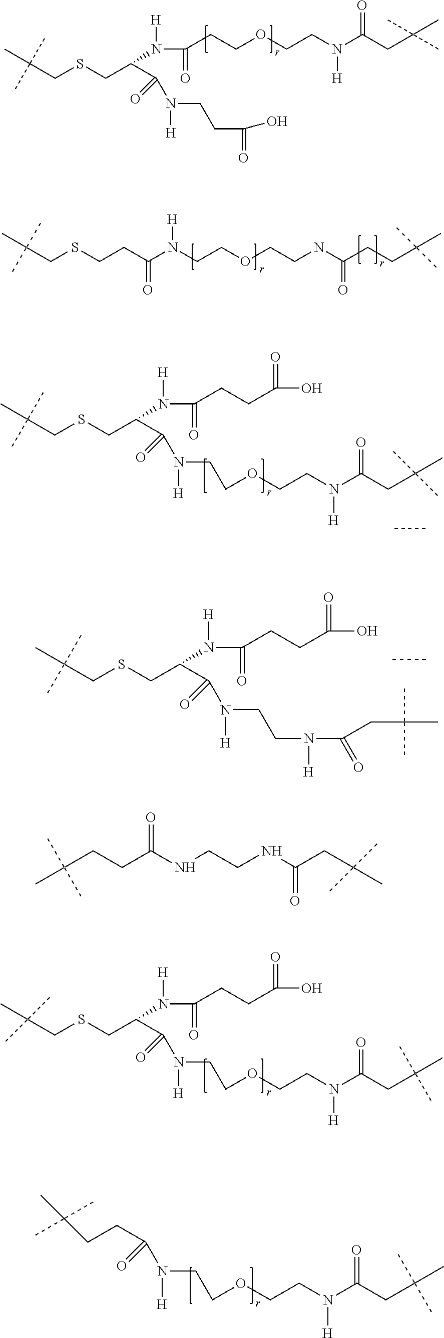

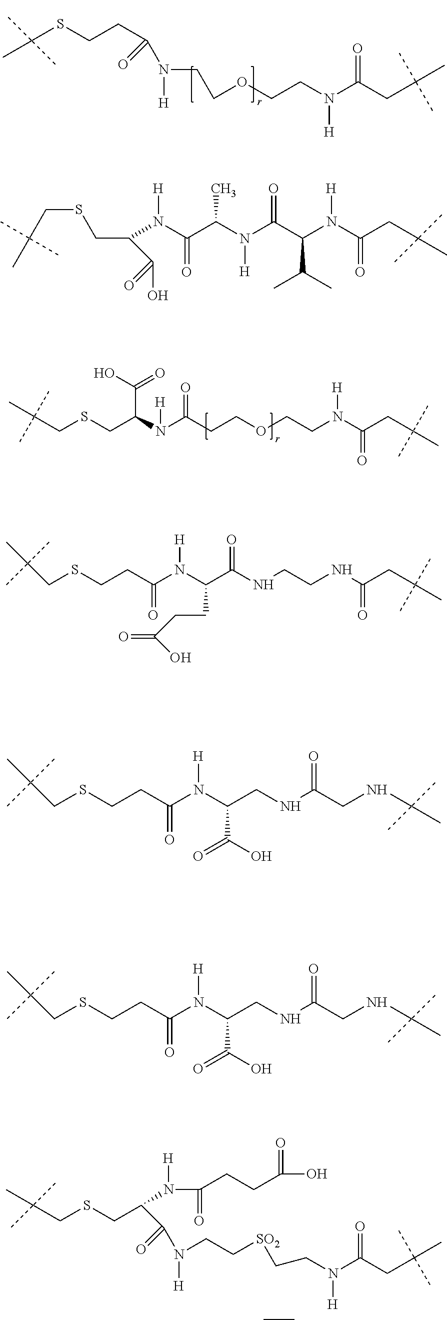

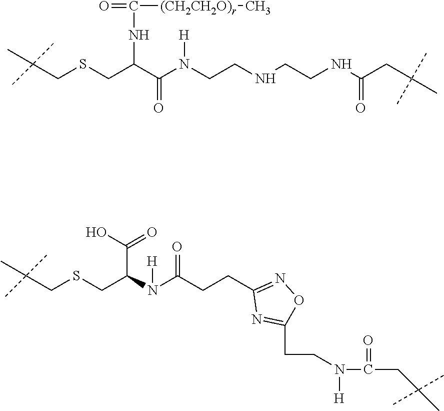

Further examples of L1 are given in Table C, in which this group is highlighted in a box.

Examples of a linker moiety L1 are given in Tables A and A′ below. The table furthermore states with which group L2 these examples of L1 are preferably combined, and also the preferred coupling point (R¹ or R³ or R⁴) and the preferred value for m, this is whether there is a carbonyl group in front of L1 or not (cf. §—(CO)m-L1-L2-§§). These linkers are preferably coupled to a cysteine residue. If L2 is a succinimide or derived therefrom, this imide may also be fully or partially in the form of the hydrolysed open-chain succinamide, as described above. Depending on L1, this hydrolysis to open-chain succinamides may be more or less pronounced or not present at all.

TABLE A

| Subst. | m | L1 | L2 |
|---|---|---|---|
| R¹ | 1 | 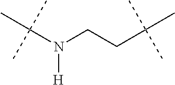 | 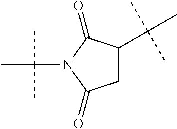 |
| R¹ | 1 | 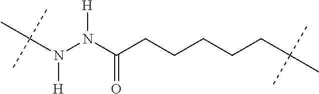 | 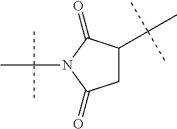 |
| R¹ | 1 | 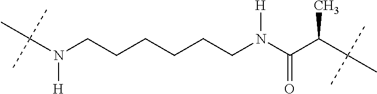 | 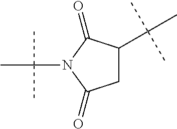 |
| R¹ | 1 | 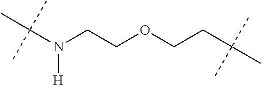 | 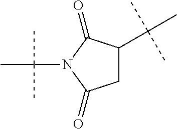 |
| R¹ | 1 | 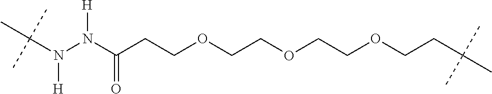 | 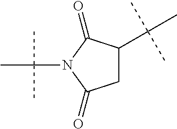 |
| R¹ | 1 | 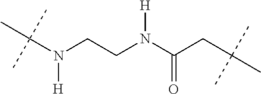 | 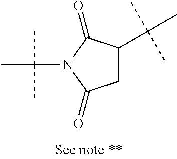 |
| R¹ | 1 | 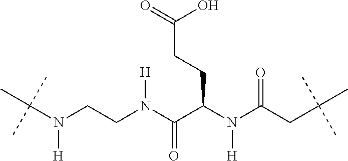 | 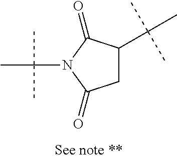 |
| R¹ | 1 | 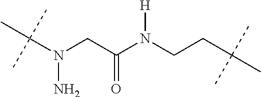 | 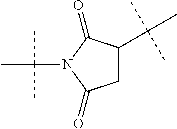 |
| R¹ | 1 | 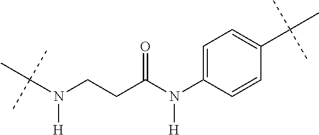 | 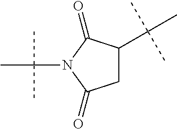 |
| R¹ | 1 | 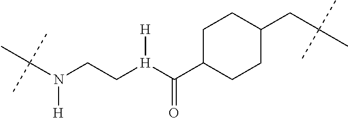 | 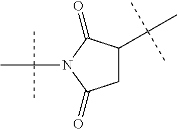 |
| R¹ | 1 | 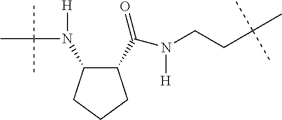 | 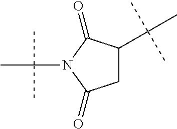 |
| R¹ | 1 | 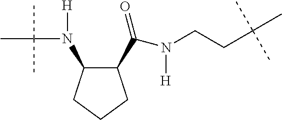 | 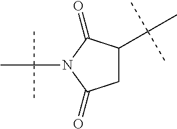 |
| R¹ | 1 | 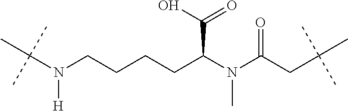 | 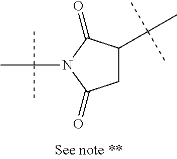 |
| R¹ | 1 | 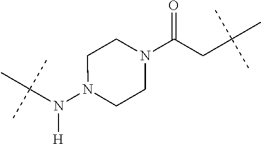 | 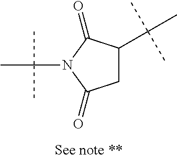 |
| R¹ | 1 | 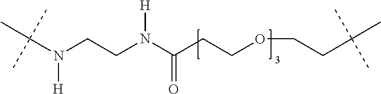 | 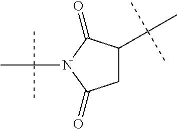 |
| R¹ | 1 | 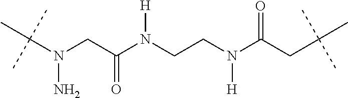 | 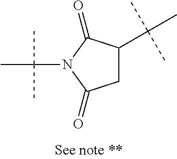 |
| R¹ | 1 | 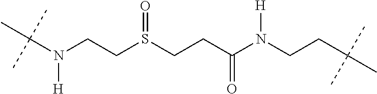 | 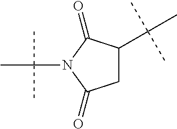 |
| R¹ | 1 | 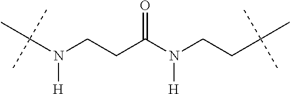 | 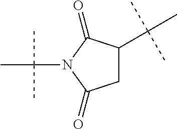 |
| R¹ | 1 | 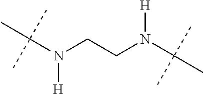 | 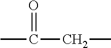 |
| R¹ | 1 | 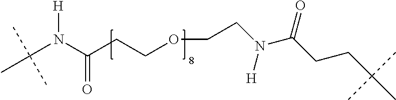 | 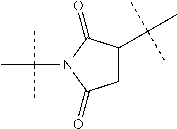 |
| R³ | 0 | 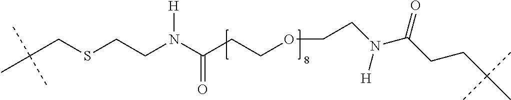 | 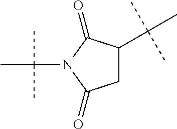 |
| R¹ | 1 | 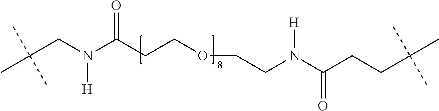 | 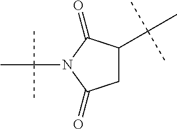 |
| R³ | 0 | 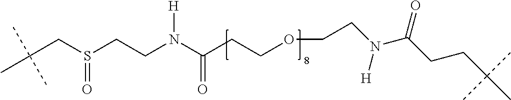 | 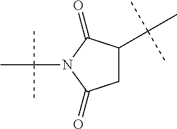 |
| R¹ | 1 | 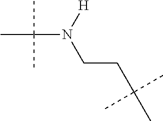 | 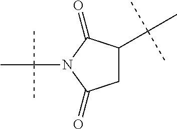 |
| R¹ | 0 | 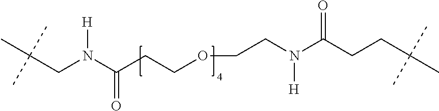 | 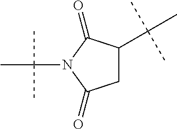 |
| R³ | 0 | 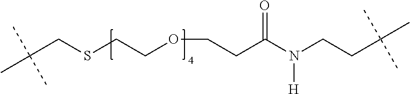 | 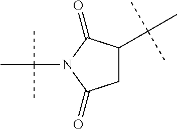 |
| R³ | 0 | 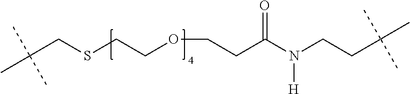 | 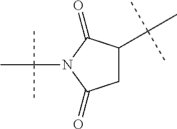 |
| R¹ | 1 | 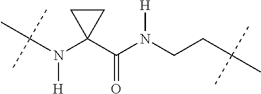 | 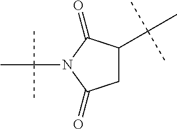 |
| R³ | 0 | 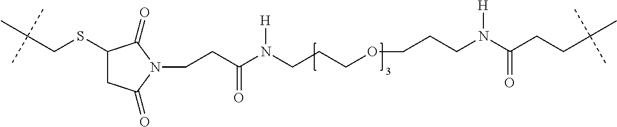 | 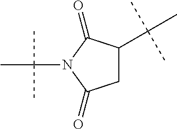 |
| R³ | 0 | 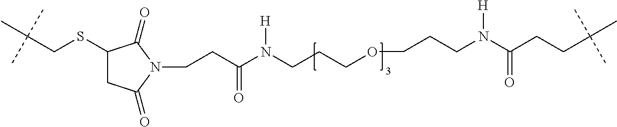 | 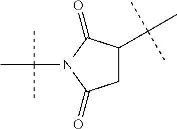 |
| R³ | 0 | 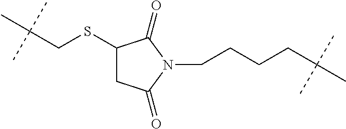 | 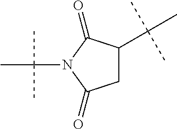 |
| R³ | 0 | 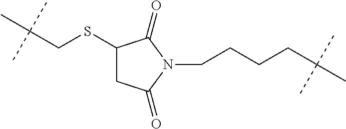 | 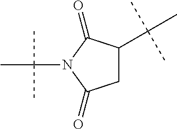 |
| R¹ | 1 | 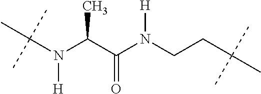 | 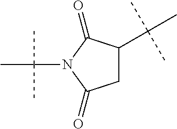 |
| R¹ | 1 | 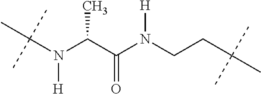 | 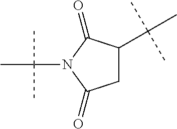 |
| R³ | 0 | 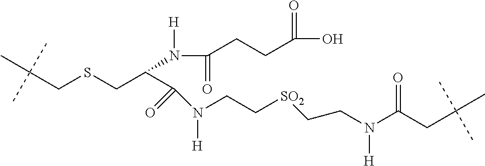 | 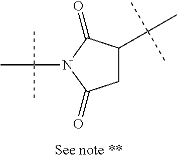 |
| R³ | 0 | 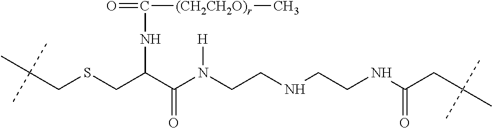 | 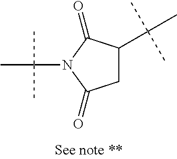 |
| R³ | 0 | 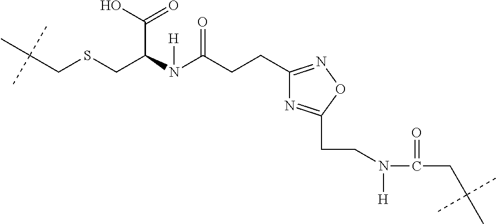 | 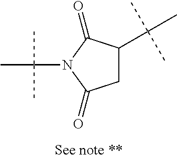 |

** With particular preference, the linkers L1 given in these rows are attached to a linker L2 selected from: 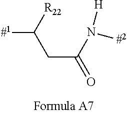
and/or
ss 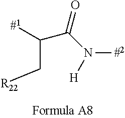
where #¹ denotes the point of attachment to the sulphur atom of the binder, #² denotes the point of attachment to group L¹, R²² preferably represents COOH. In a conjugate according to the invention or in a mixture of the conjugates according to the invention, the bonds to a cysteine residue of the binder are present, to an extent of preferably more than 80%, particularly preferably more than 90% (in each case based on the total number of bonds of the linker to the binder), particularly preferably as one of the two structures of the formula A7 or A8. Here, the structures of the formula A7 or A8 are generally present together, preferably in a ratio of from 60:40 to 40:60, based on the number of bonds to the binder. The remaining bonds are then present as the structure

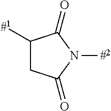

TABLE A‘

| Subst. | m | L1 | L2 |
|---|---|---|---|
| R¹ | 1 | 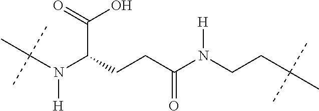 | 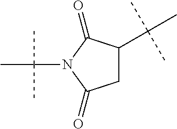 |
| R¹ | 1 | 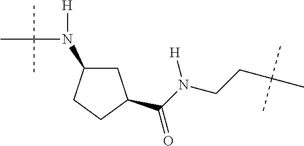 | 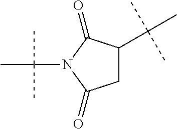 |
| R¹ | 1 | 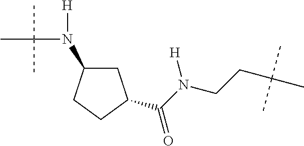 | 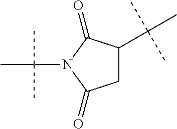 |
| R¹ | 1 | 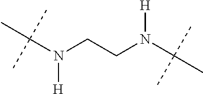 | 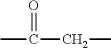 |
| R¹ | 1 | 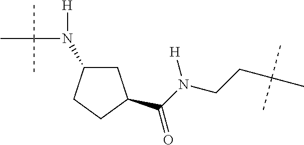 | 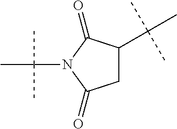 |
| R¹ | 1 | 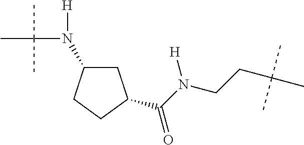 | 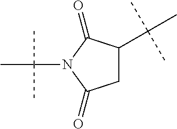 |
| R³ | 0 | 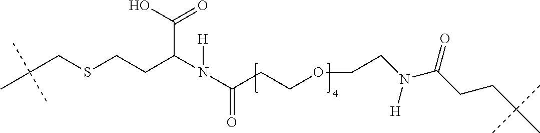 | 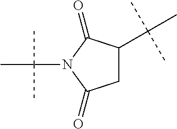 |
| R³ | 0 | 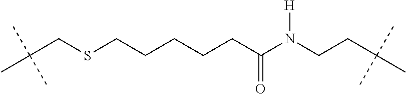 | 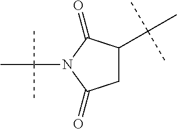 |
| R³ | 0 | 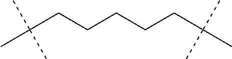 | 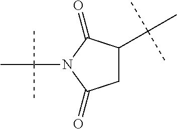 |
| R³ | 0 | 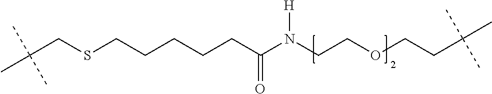 | 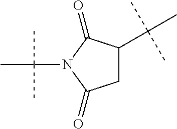 |
| R³ | 0 | 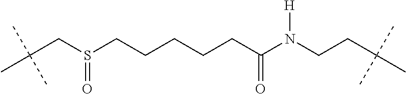 | 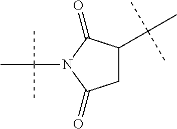 |
| R¹ | 1 | 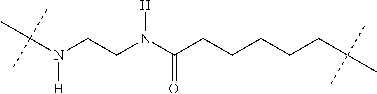 | 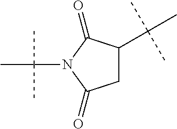 |
| R¹ | 1 | 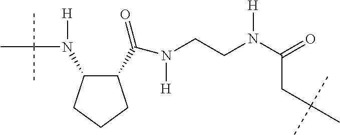 | 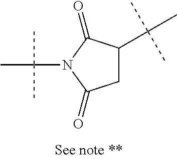 |
| R¹ | 1 | 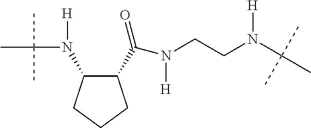 | 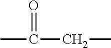 |
| R¹ | 1 | 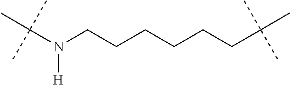 | 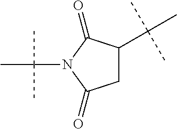 |
| R¹ | 1 | 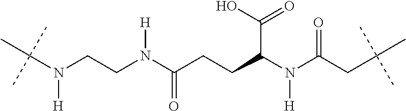 | 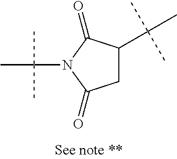 |
| R¹ | 1 | 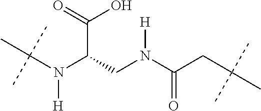 | 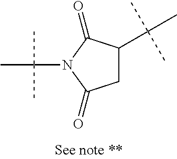 |
| R¹ | 1 | 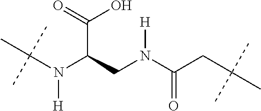 | 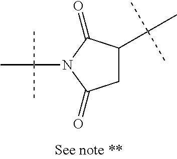 |
| R¹ | 0 | 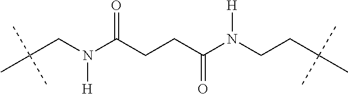 | 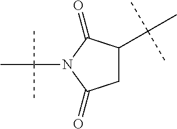 |
| R¹ | 1 | 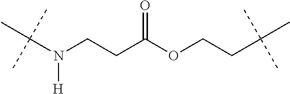 | 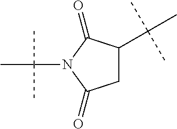 |
| R¹ | 1 | 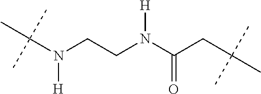 | 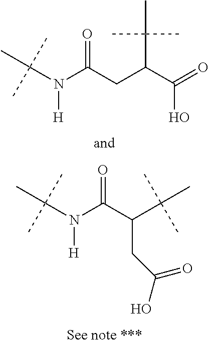 |
| R¹ | 1 | 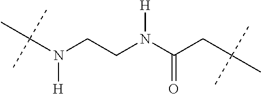 | 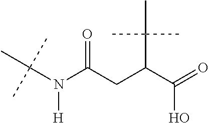 |
| R¹ | 1 | 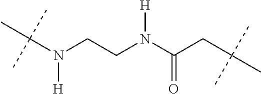 | 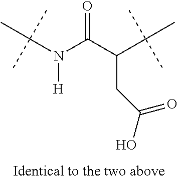 |
| R¹ | 1 | 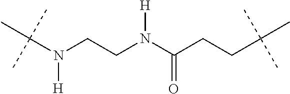 | 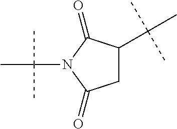 |
| R³ | 0 | 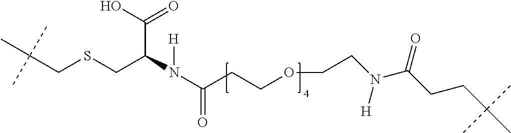 | 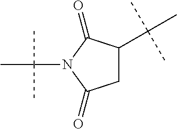 |
| R³ | 0 | 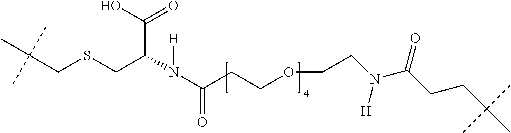 | 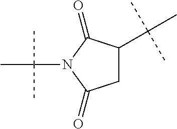 |
| R³ | 0 | 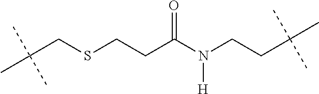 | 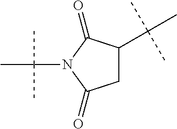 |
| R³ | 0 | 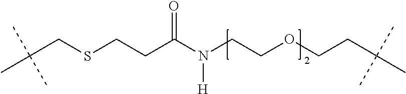 | 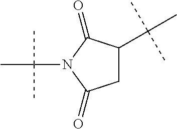 |
| R³ | 0 | 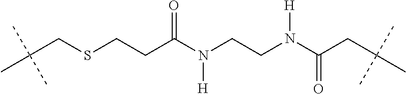 | 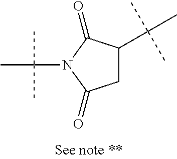 |
| R³ | 0 | 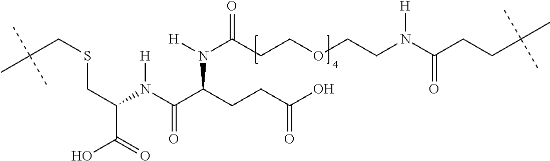 | 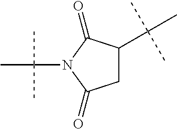 |
| R³ | 0 | 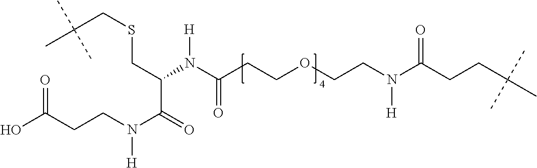 | 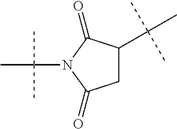 |
| R³ | 0 | 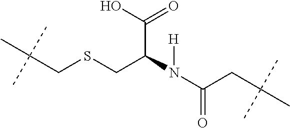 | 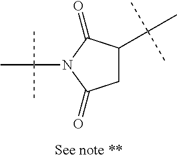 |
| R³ | 0 | 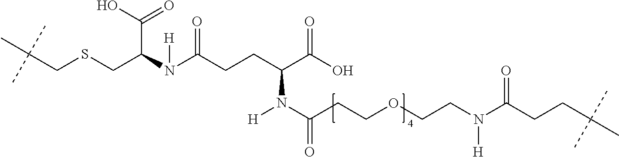 | 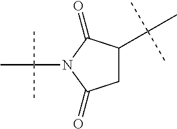 |
| R² | 0 | 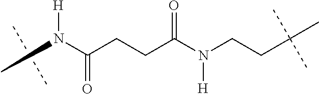 | 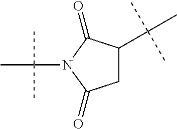 |
| R¹ | 1 | 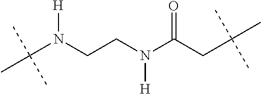 | 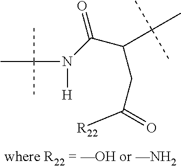 |
| R¹ | 1 | 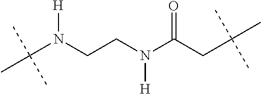 | 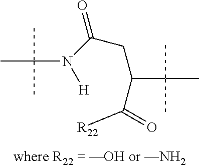 |
| R¹ | 1 | 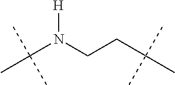 | 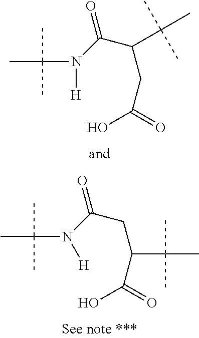 |
| R¹ | 1 | 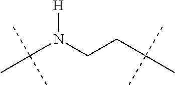 | 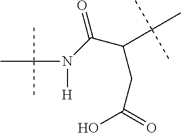 |
| R¹ | 1 | 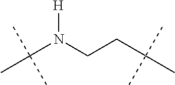 | 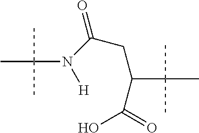 |
| R¹ | 1 | 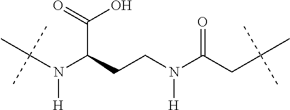 | 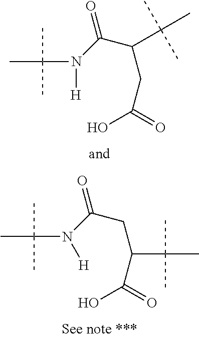 |
| R¹ | 1 | 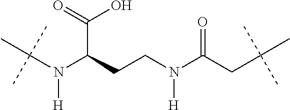 | 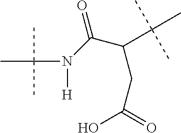 |
| R¹ | 1 | 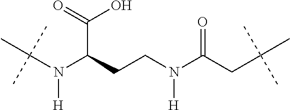 | 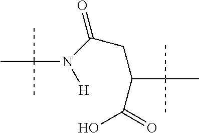 |
| R³ | 0 | 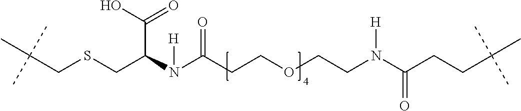 | 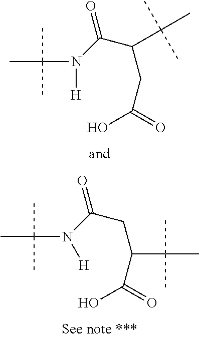 |
| R³ | 0 | 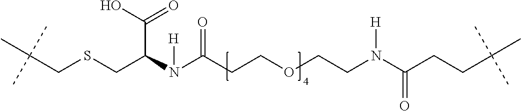 | 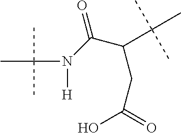 |
| R³ | 0 | 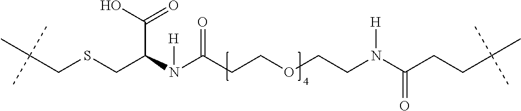 | 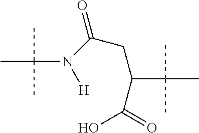 |
| R³ | 0 | 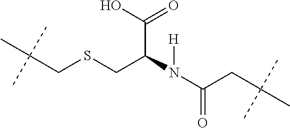 | 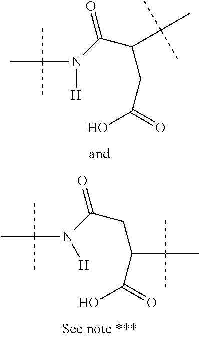 |
| R³ | 0 | 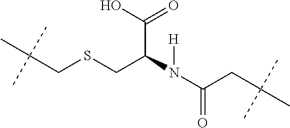 | 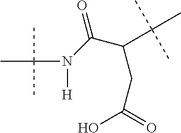 |
| R³ | 0 | 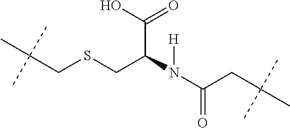 | 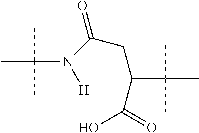 |
| R³ | 0 | 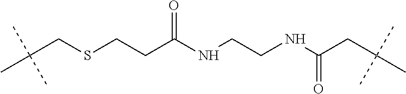 | 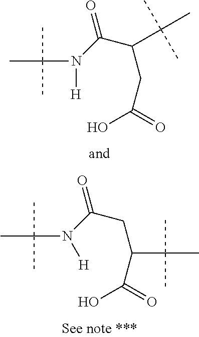 |
| R³ | 0 | 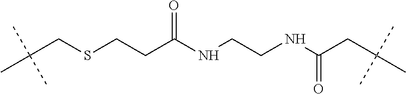 | 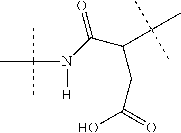 |
| R³ | 0 | 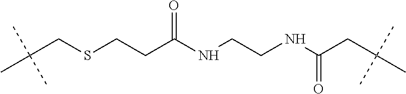 | 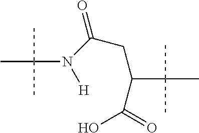 |
| R¹ | 1 | 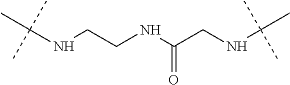 | 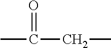 |
| R¹ | 1 | 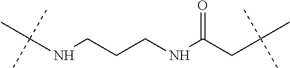 | 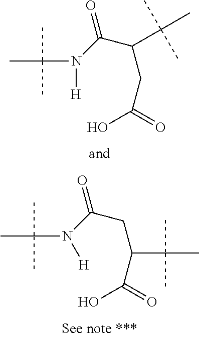 |
| R¹ | 1 | 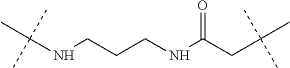 | 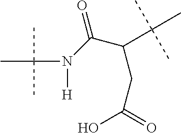 |
| R¹ | 1 | 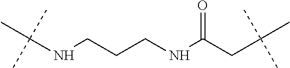 | 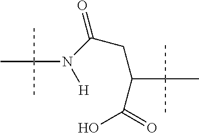 |
| R¹ | 1 | 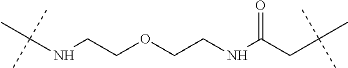 | 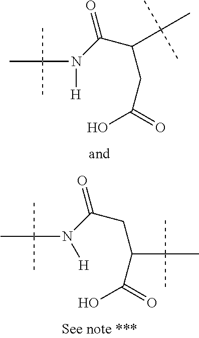 |
| R¹ | 1 | 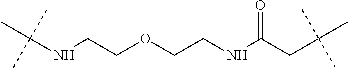 | 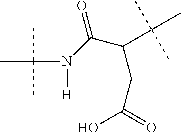 |
| R¹ | 1 | 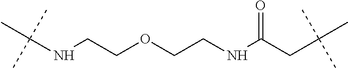 | 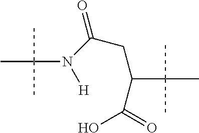 |
| R³ | 0 | 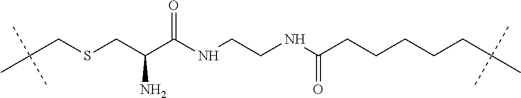 | 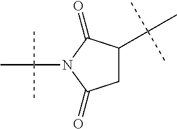 |
| R1 | 0 | 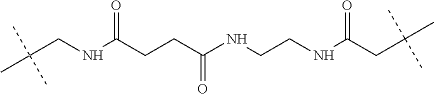 | 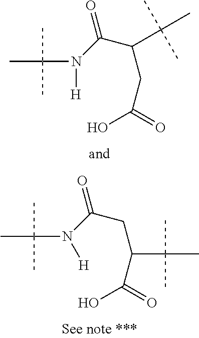 |
| R1 | 0 | 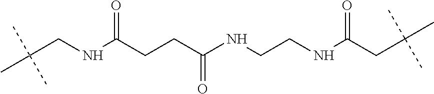 | 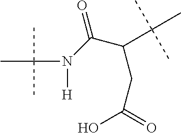 |
| R1 | 0 | 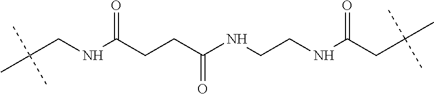 | 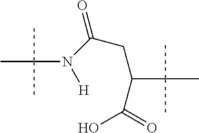 |
| R1 | 1 | 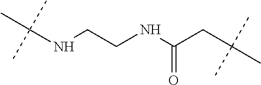 | 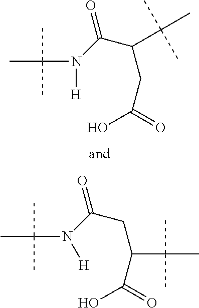 |
| R1 | 1 | 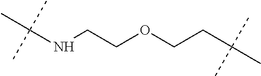 | 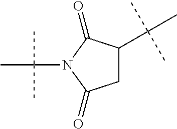 |
| R1 | 1 | 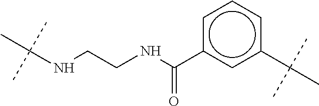 | 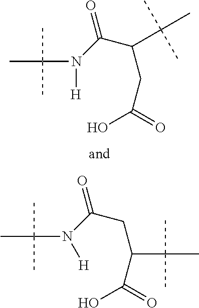 |
| R¹ | 1 | 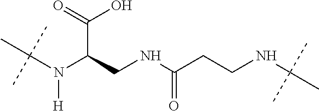 | 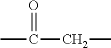 |
| R¹ | 1 | 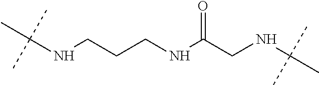 | 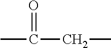 |
| R¹ | 1 | 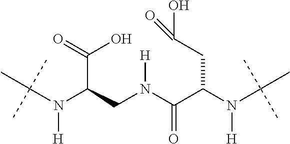 | 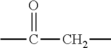 |
| R¹ | 1 | 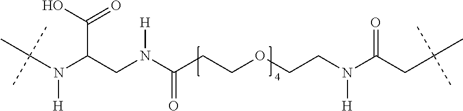 | 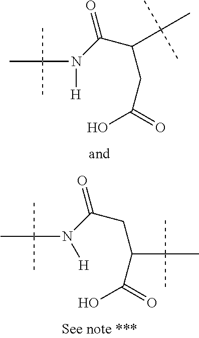 |
| R¹ | 1 | 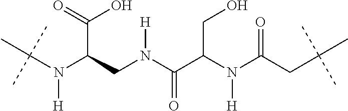 | 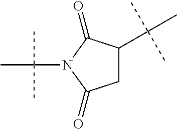 |
| R¹ | 1 | 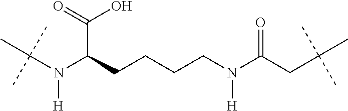 | 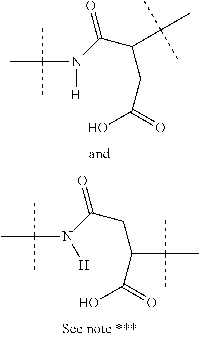 |
| R¹ | 1 | 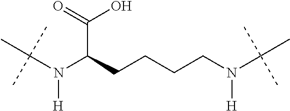 | 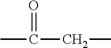 |
| R⁴ | 0 | 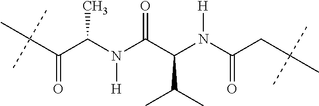 | 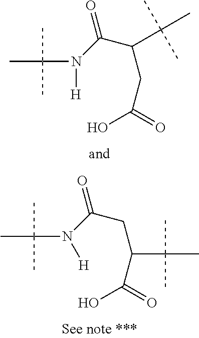 |
| R¹ | 1 | 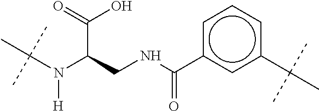 | 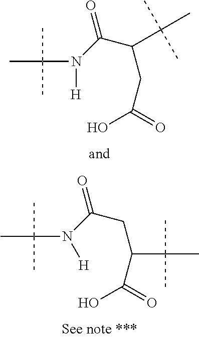 |
| R⁴ | 0 | 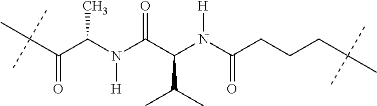 | 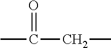 |
| R¹ | 1 | 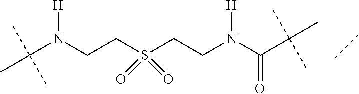 | 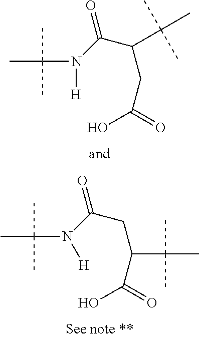 |
| R³ | 0 | 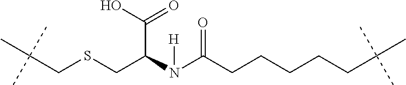 | 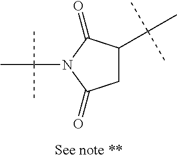 |
| R¹ | 1 | 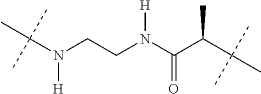 | 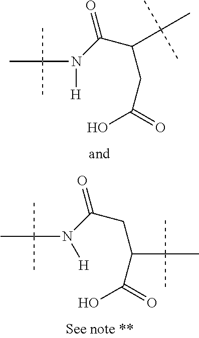 |
| R³ | 0 | 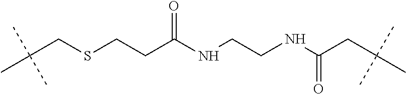 | 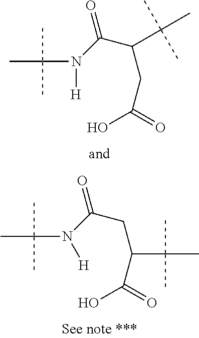 |
| R³ | 0 | 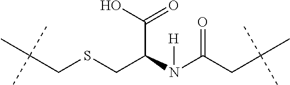 | 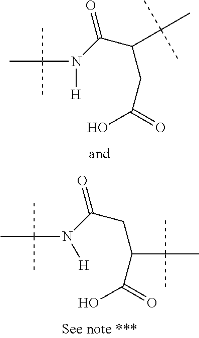 |
| R³ | 0 | 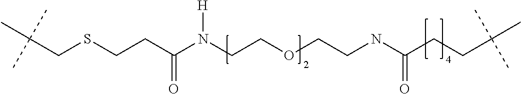 | 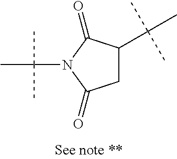 |
| R³ | 0 | 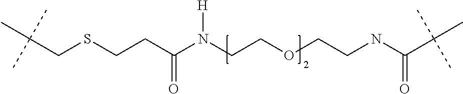 | 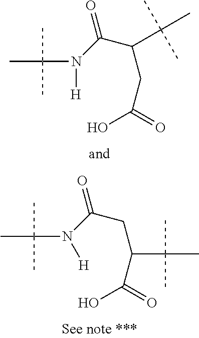 |
| R³ | 0 | 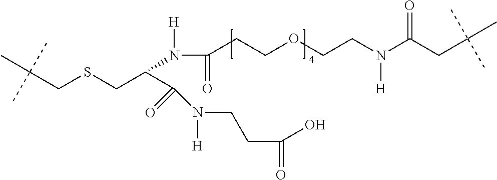 | 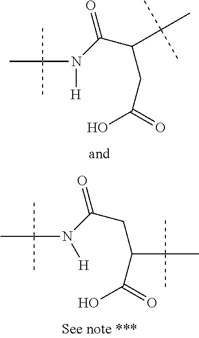 |
| R³ | 0 | 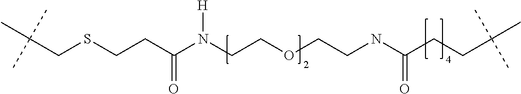 | 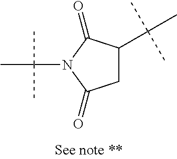 |
| R³ | 0 | 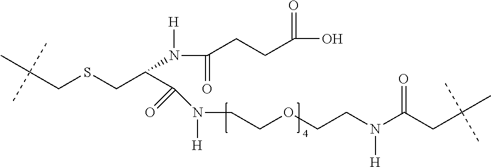 | 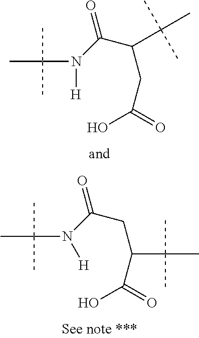 |
| R³ | 0 | 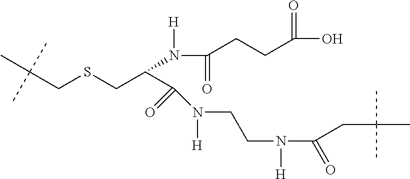 | 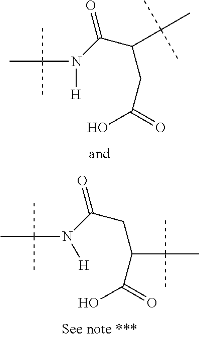 |
| R³ | 0 | 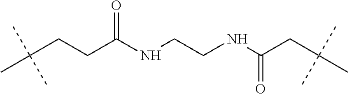 | 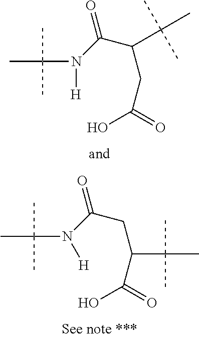 |
| R³ | 0 | 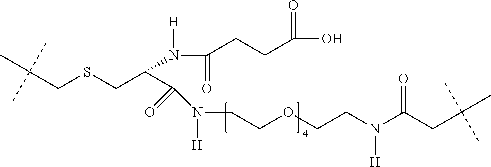 | 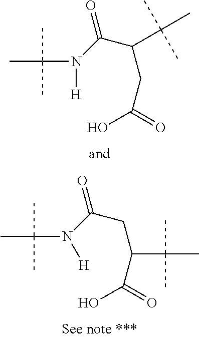 |
| R³ | 0 | 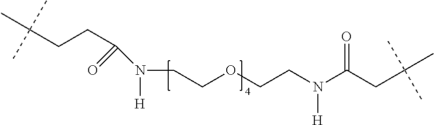 | 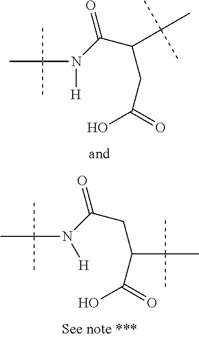 |
| R³ | 0 | 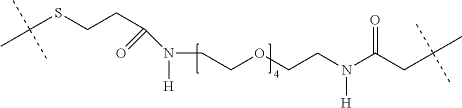 | 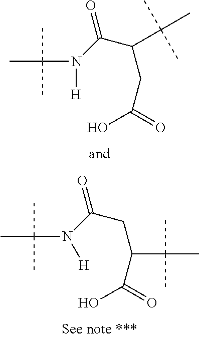 |
| R³ | 0 | 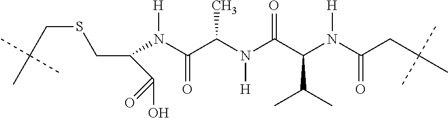 | 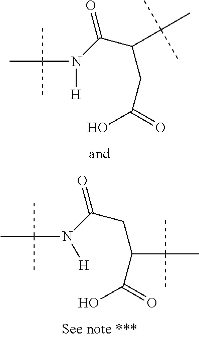 |
| R³ | 0 | 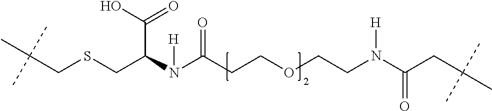 | 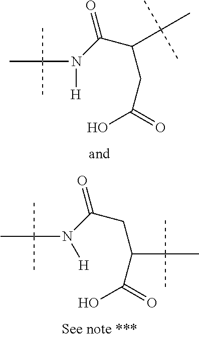 |
| R³ | 0 | 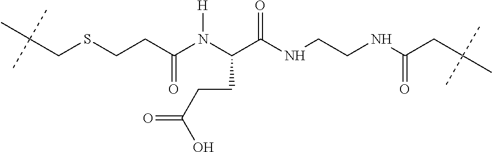 | 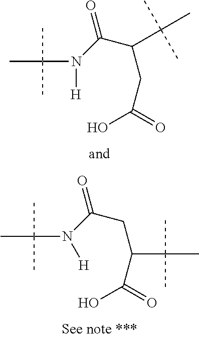 |
| R³ | 0 | 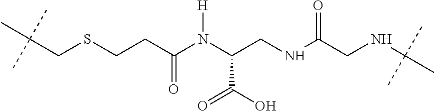 | 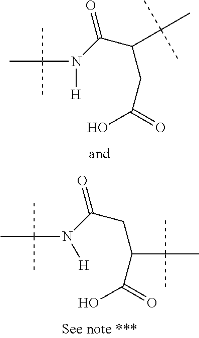 |
| R³ | 0 | 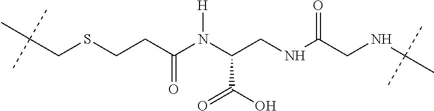 | 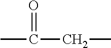 |

** See note ** for Table A.
*** When this structure L2 is present, there may simultaneously be a structure L2 of the formula below:

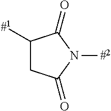

Examples of conjugates having corresponding linkers have the following structures, where X1 represents CH, X2 represents C and X3 represents N and L1 has the meaning given above, L2 and L3 have the same meaning as L1, AK1 represents a moderately agonistically or non-agonistically acting anti-TWEAKR antibody such as ITEM-4 and chimeric or humanized variants of ITEM-4 attached via a cysteine residue and n is a number from 1 to 10. With particular preference, AK1 is a humanized or chimeric monoclonal ITEM-4 anti-TWEAKR antibody. Particular preference is given to the humanized or chimeric ITEM-4 variants: TPP-7005, TPP-7006, TPP-7007, TPP-7053, TPP-7073, TPP-7074, TPP-7075 and TPP-7076.

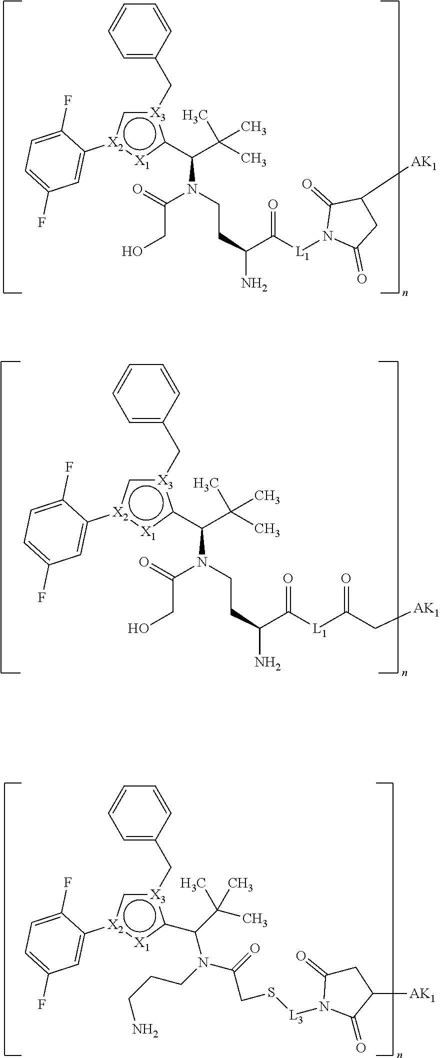

If the linker is attached to a lysine side chain or a lysine residue, it preferably has the formula below:

-§—(SG)ₓ-L4-CO-§§

where
§ represents the bond to the active compound molecule and
§§ represents the bond to the binder peptide or protein,
x represents 0 or 1,
SG represents a cleavable group, preferably a 2-8 oligopeptide, particularly preferably a dipeptide, and
L4 represents a single bond or a group —(CO)_{y}-G4-, where y represents 0 or 1, and
G4 represents a straight-chain or branched hydrocarbon chain having 1 to 100 carbon atoms from arylene groups and/or straight-chain and/or branched and/or cyclic alkylene groups and which may be interrupted once or more than once by one or more of the groups —O—, —S—, —SO—, SO₂, —NH—, —CO—, —NHCO—, —CONH—, —NMe-, —NHNH—, —SO₂NHNH—, —CONHNH— and a 5- to 10-membered aromatic or non-aromatic heterocycle having up to 4 heteroatoms selected from the group
consisting of N, O and S, —SO— or —SO₂— (preferably

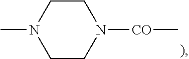

where the side chains, if present, may be substituted by —NHCONH₂, —COOH, —OH, —NH₂, NH—CNNH₂, sulphonamide, sulphone, sulphoxide or sulphonic acid.

Table B below gives examples of linkers to a lysine residue. The table furthermore gives the preferred coupling point (R¹-R⁵). The first column furthermore states the example numbers in which the corresponding linkers are used.

TABLE B
lysine linker -§-(SG)ₓ—L4—CO-§§

| Ex. | Subst. | (SG)ₓ—L4 |
|---|---|---|
| 194, 294 | R⁴ | 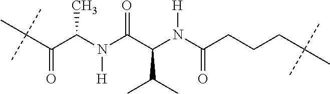 |

Examples of conjugates having corresponding linkers have the following structures, where X1 represents CH, X2 represents C and X3 represents N and L4 has the meaning given above, AK2 represents a moderately agonistically or non-agonistically acting anti-TWEAKR antibody such as ITEM-4 and chimeric or humanized variants of ITEM-4 attached via a lysine residue and n is a number from 1 to 10. Particularly preferably, AK2 is a humanized or chimeric monoclonal ITEM-4 anti-TWEAKR antibody. Particular preference is given to the humanized or chimeric ITEM-4 variants: TPP-7005, TPP-7006, TPP-7007, TPP-7053, TPP-7073, TPP-7074, TPP-7075 and TPP-7076.

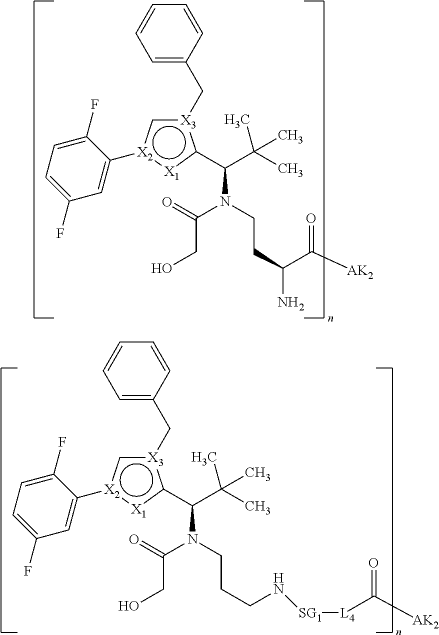

Preference according to the invention is furthermore given to the basic structure (i), (ii) or (iv), where SG1 or SG represents a group which can be cleaved by a protease and L1 and L2 have the meanings given above. Particular preference is given to the following groups:

-Val-Ala-CONH— (hereby cleavage of the amide bond at the C-terminal amide of alanine)
—NH-Val-Lys-CONH— (cleavage of the amide bond at the C-terminal amide of lysine)
—NH-Val-Cit-CONH— (cleavage of the amide bond at the C-terminal amide of citrulline)
—NH-Phe-Lys-CONH (cleavage of the amide bond at the C-terminal amide of lysine)
—NH-Ala-Lys-CONH— (cleavage of the amide bond at the C-terminal amide of lysine)
—NH-Ala-Cit-CONH— (cleavage of the amide bond at the C-terminal amide of citrulline)
SG1 or SG is particularly preferably

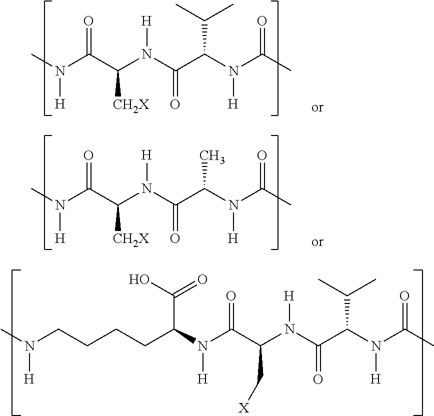

where X represents H or a C₁₋₁₀-alkyl group which may optionally be substituted by —NHCONH₂, —COOH, —OH, NH₂, —NH—CNNH₂ or sulphonic acid.

Table C below gives examples of a linker moiety -SG1-L1- or -L1-SG-L1-, where SG1 and SG are groups which can be cleaved by a protease. Table C furthermore states with which group L2 these examples of -SG1-L1- and -L1-SG-L1-are preferably combined, and also the preferred coupling point (R¹-R⁵) and the preferred value for m, thus whether there is a carbonyl group in front of L1 or not (cf. §—(CO)m-L1-L2-§§). These linkers are preferably coupled to a cysteine residue. The L1 group is highlighted in a box. However, these groups L1 can be replaced by one of the groups L1 given for formula §—(CO)m-L1-L2-§§ above. If L2 is a succinamide or derived therefrom, this amide may also be fully or partially in the form of the hydrolysed open-chain succinamide, as described above.

TABLE C

| Sub | | | |
|---|---|---|---|
| St. | m | —SG1—L1— or —L1—SG—L1— | L2 |
| R¹ | 1 | 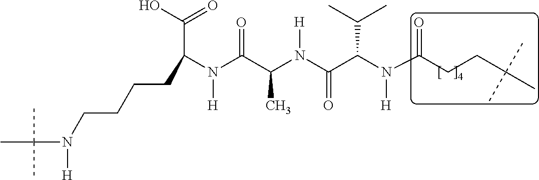 | 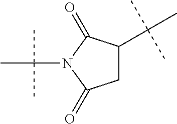 |
| R¹ | 1 | 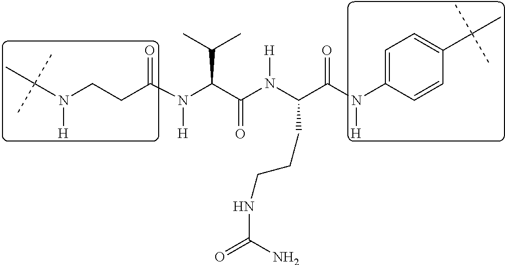 | 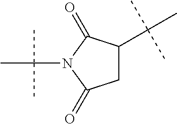 |
| R¹ | 1 | 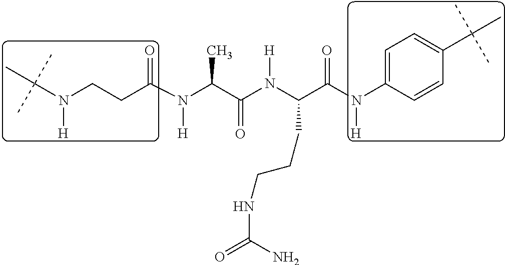 | 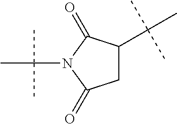 |
| R¹ | 1 | 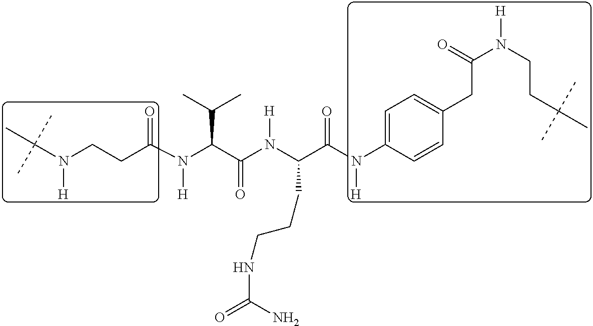 | 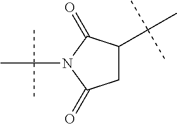 |
| R¹ | 1 | 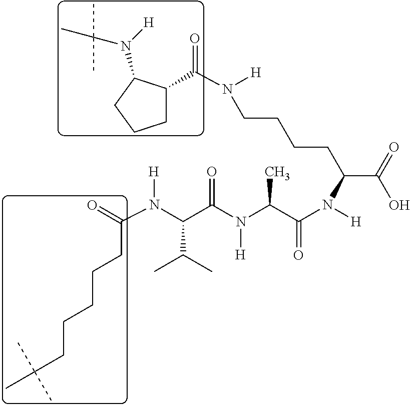 | 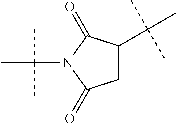 |
| R¹ | 1 | 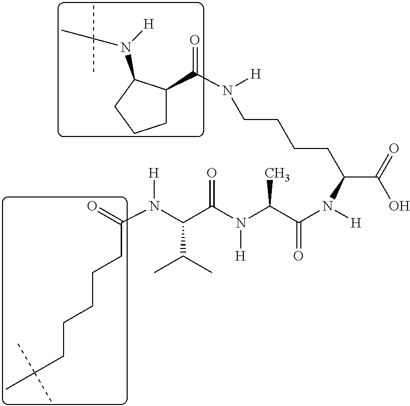 | 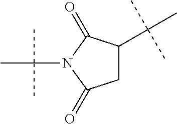 |
| R¹ | 1 | 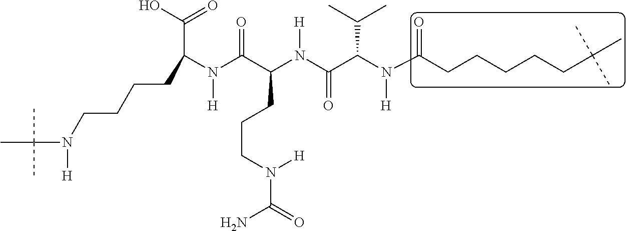 | 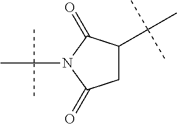 |
| R¹ | 1 | 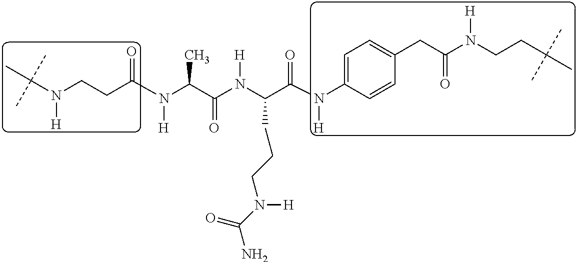 | 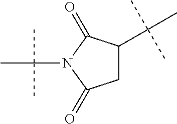 |
| R¹ | 1 | 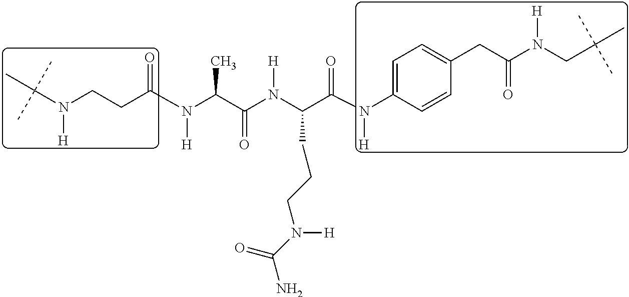 | 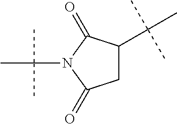 |
| R¹ | 1 | 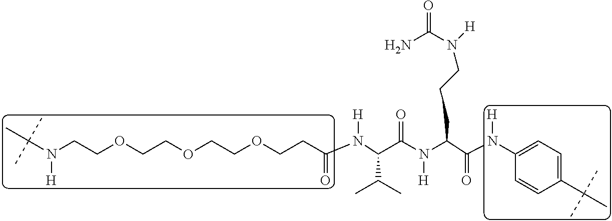 | 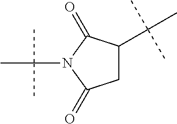 |
| R¹ | 1 | 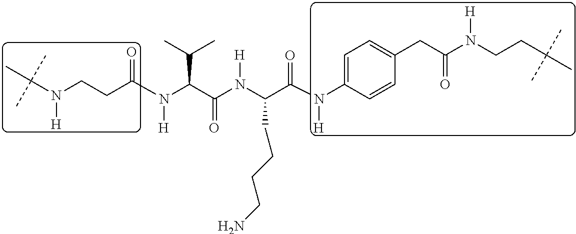 | 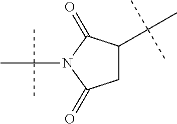 |
| R¹ | 1 | 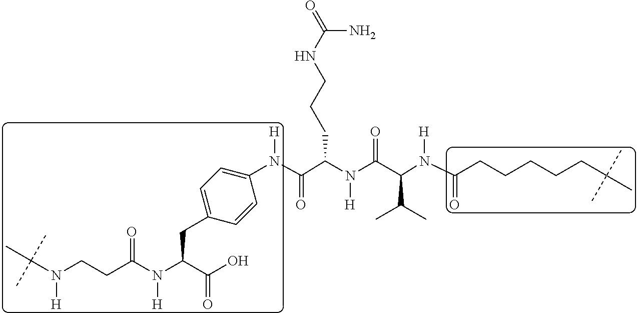 | 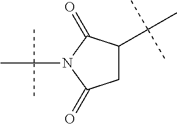 |
| R¹ | 1 | 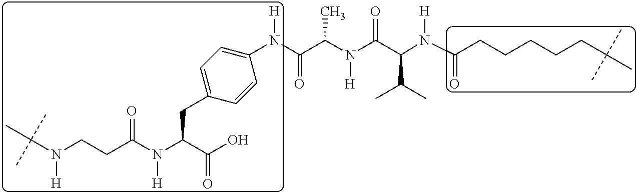 | 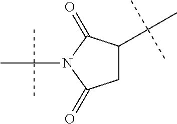 |
| R¹ | 0 | 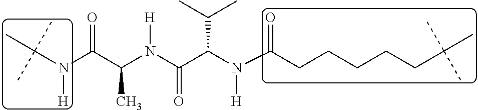 | 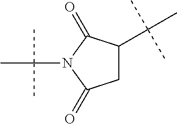 |
| R¹ | 1 | 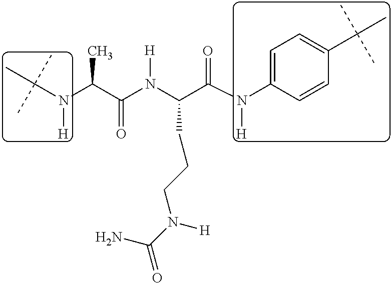 | 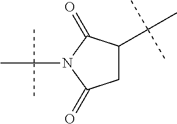 |
| R¹ | 0 | 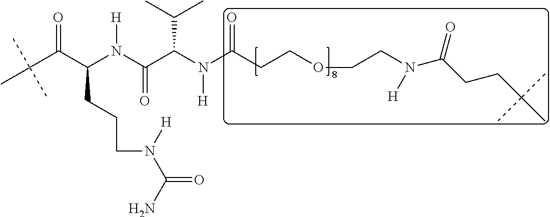 | 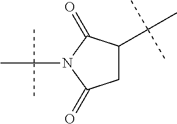 |
| R¹ | 0 | 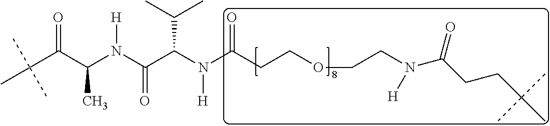 | 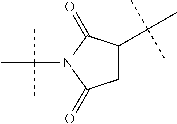 |
| R¹ | 0 | 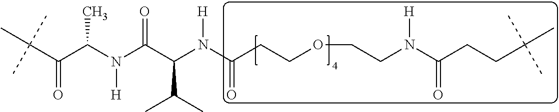 | 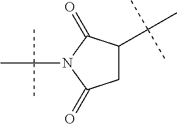 |
| R¹ | 0 | 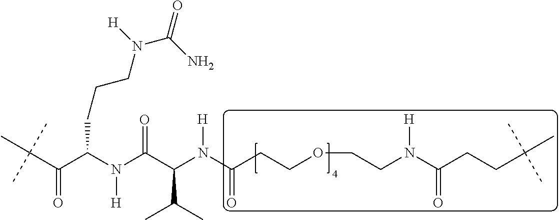 | 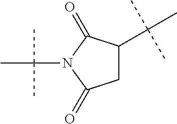 |
| R¹ | 0 | 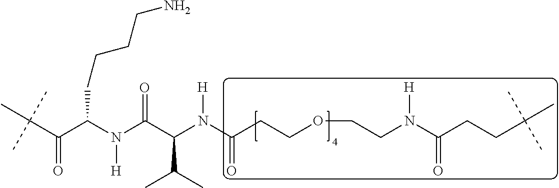 | 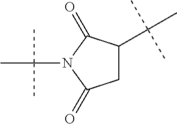 |
| R¹ | 0 | 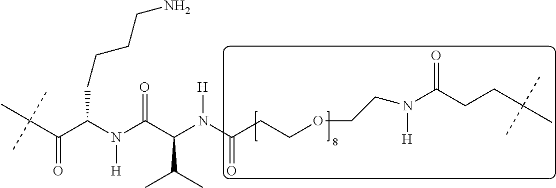 | 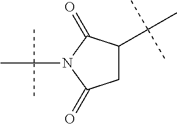 |
| R¹ | 0 | 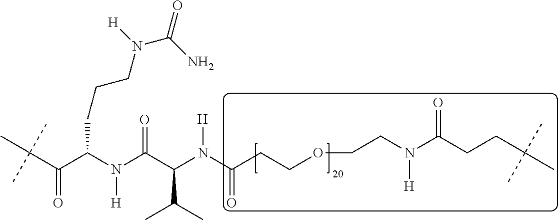 | 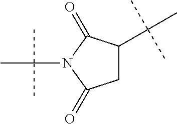 |
| R³ | 0 | 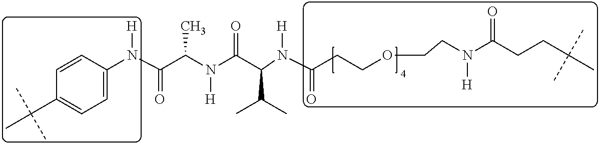 | 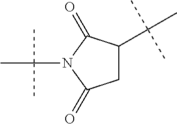 |
| R³ | 0 | 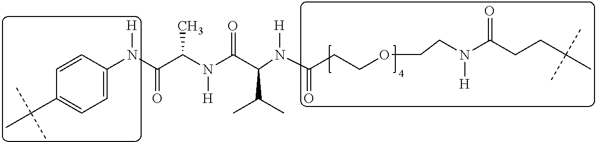 | 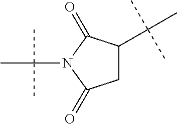 |
| R¹ | 1 | 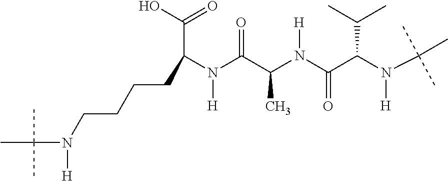 | 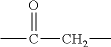 |
| R¹ | 1 | 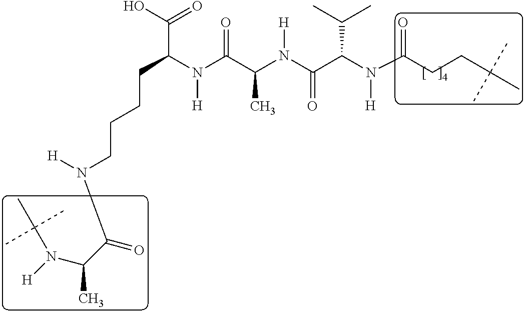 | 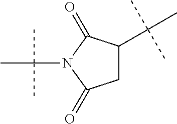 |
| R¹ | 1 | 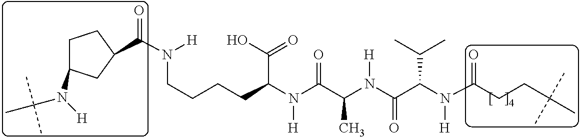 | 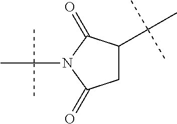 |
| R³ | 0 | 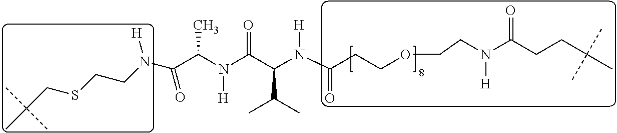 | 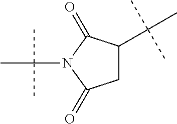 |
| R¹ | 1 | 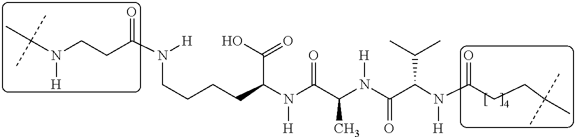 | 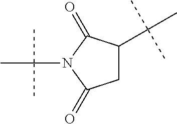 |
| R¹ | 1 | 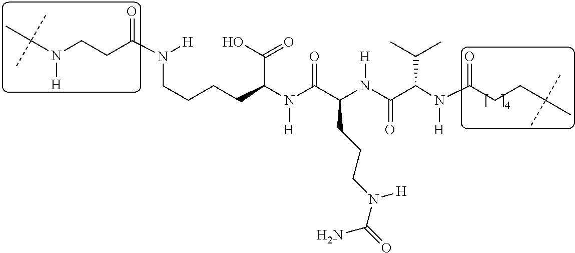 | 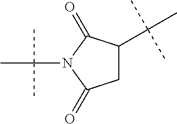 |
| R¹ | 1 | 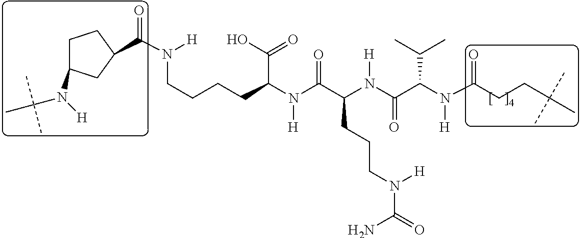 | 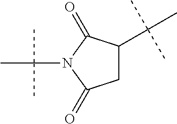 |
| R¹ | 1 | 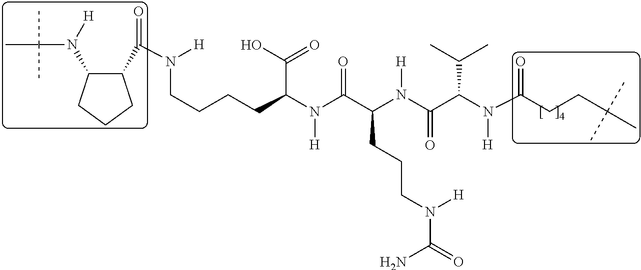 | 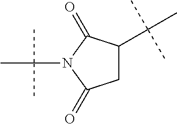 |
| R¹ | 1 | 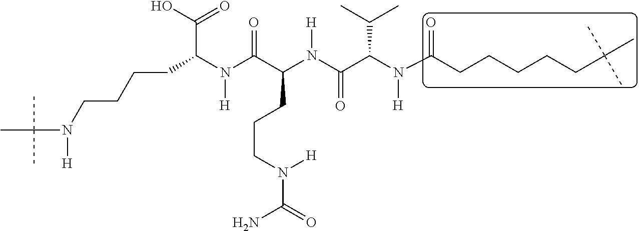 | 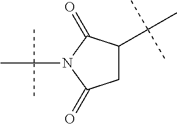 |
| R¹ | 1 | 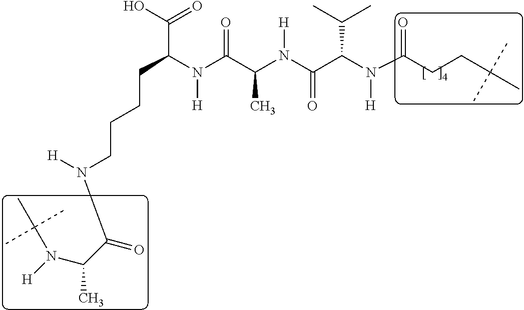 | 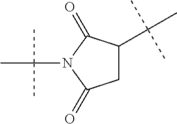 |
| R¹ | 1 | 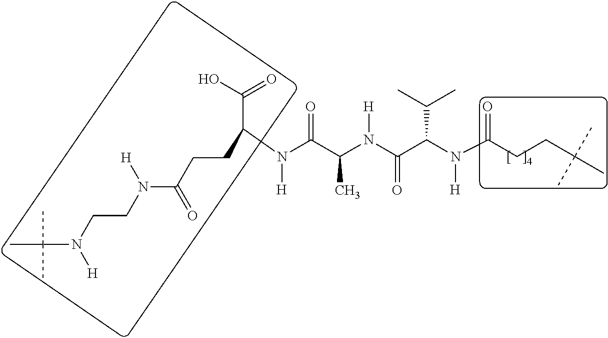 | 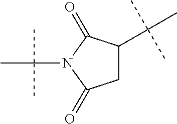 |
| R¹ | 1 | 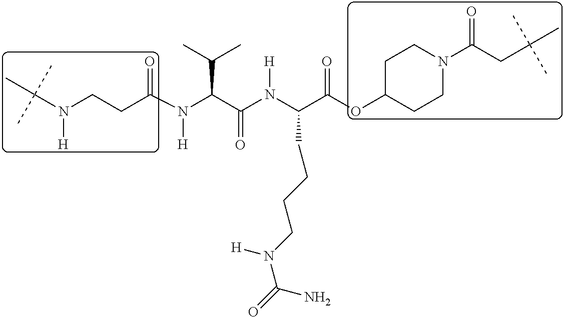 | 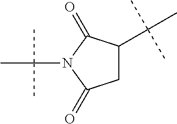 |
| R¹ | 1 | 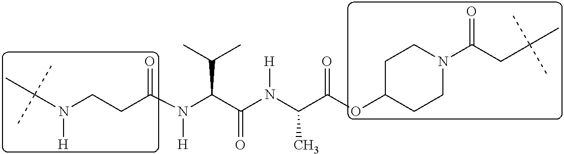 | 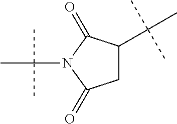 |
| R¹ | 1 | 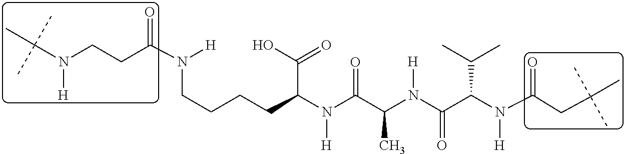 | 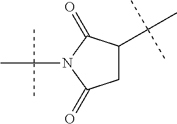 |
| R³ | 0 | 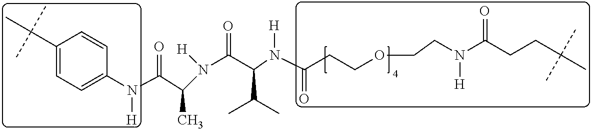 | 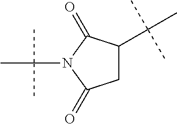 |
| R¹ | 1 | 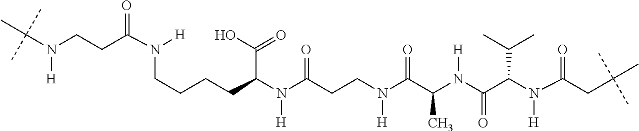 | 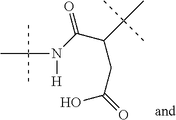 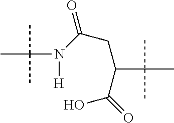 |
| R³ | 0 | 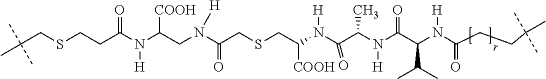 | 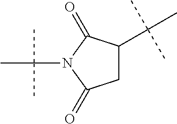 |

Examples of conjugates having basic structure (i) have the following structure, where X1 represents CH, X2 represents C and X3 represents N, L4 has the same meaning as L1, AK1 represents a moderately agonistically or non-agonistically acting anti-TWEAKR antibody such as ITEM-4 and chimeric or humanized variants of ITEM-4 attached via a cysteine residue and n is a number from 1 to 10. Particularly preferably, AK1 is a humanized or chimeric monoclonal ITEM-4 anti-TWEAKR antibody. Particular preference is given to the humanized or chimeric ITEM-4 variants: TPP-7005, TPP-7006, TPP-7007, TPP-7053, TPP-7073, TPP-7074, TPP-7075 and TPP-7076.

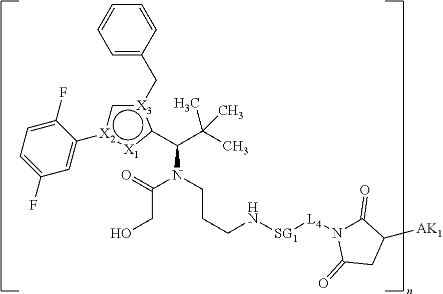

### KSP Inhibitor-Linker-Intermediates and Preparation of the Conjugates

The conjugates according to the invention are prepared by initially providing the low-molecular weight KSP inhibitor with a linker. The intermediate obtained in this manner is then reacted with the binder (preferably antibody).

Preferably, for coupling to a cysteine residue, one of the compounds below is reacted with the cysteine-containing binder such as an antibody, which is optionally partially reduced for this purpose:

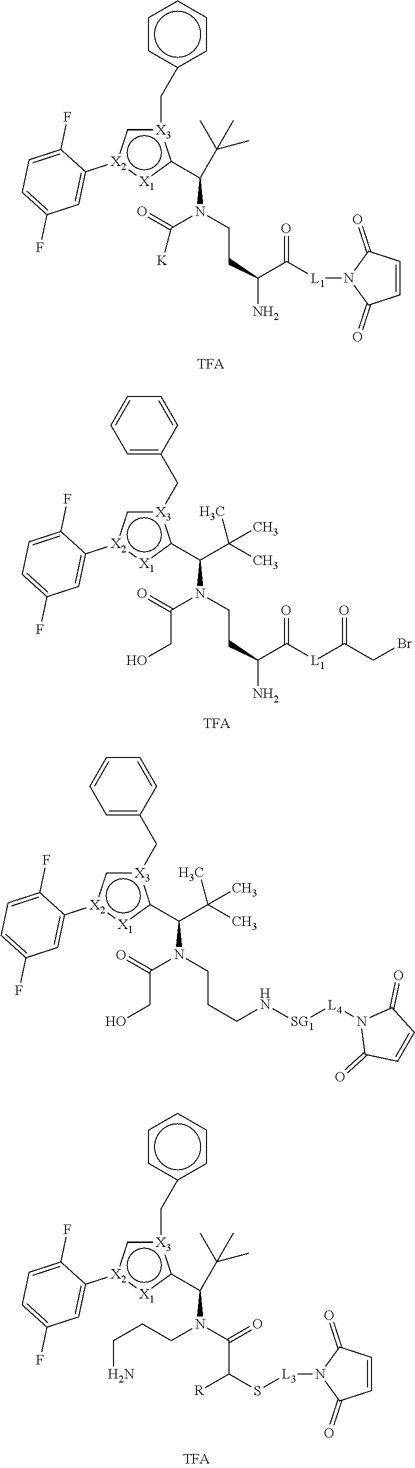

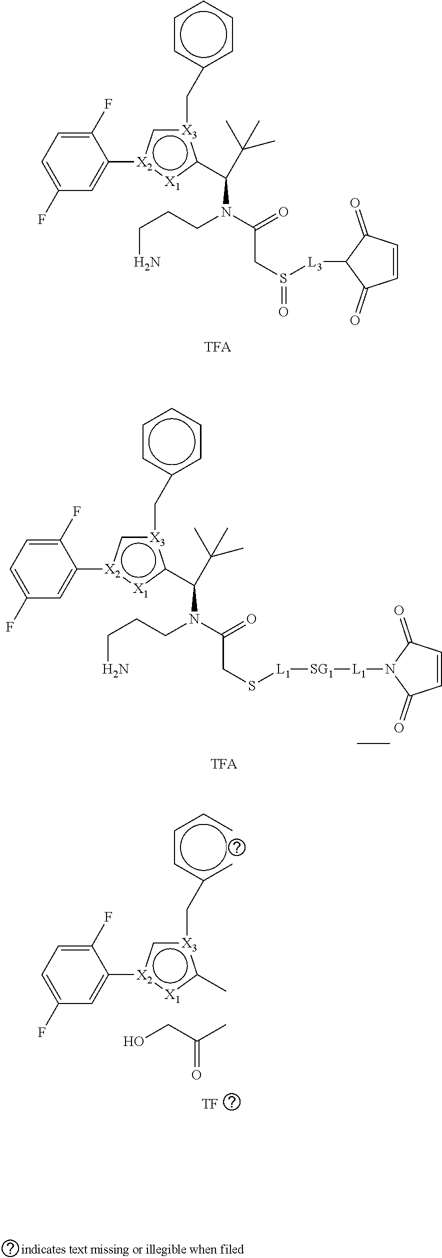

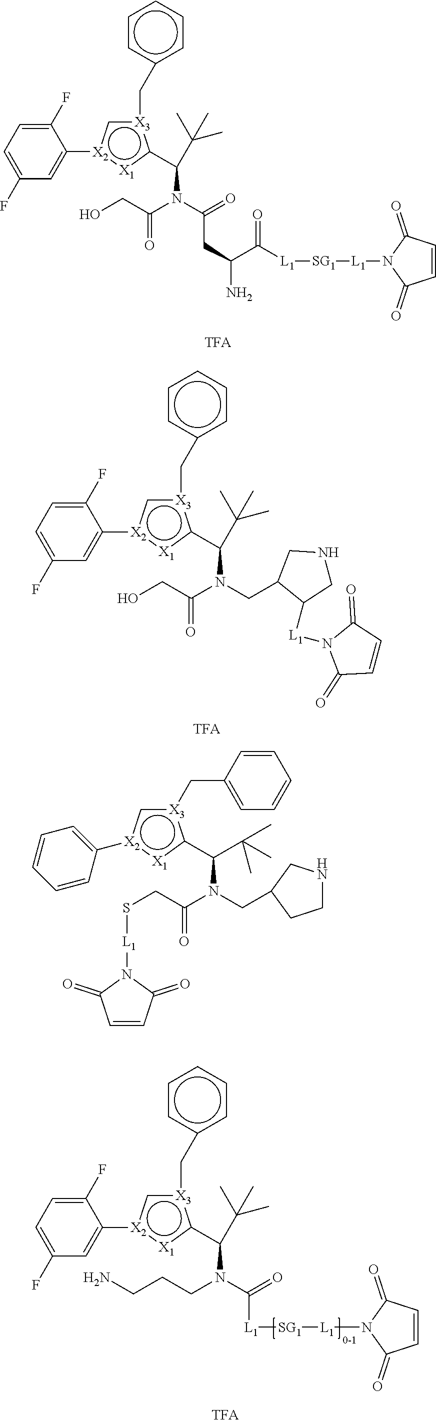

where R represents —H or —COOH,
where K represents straight-chain or branched C₁-C₆ alkyl which is optionally substituted by C₁-C₆-alkoxy or —OH, and
where X1 represents CH, X2 represents C and X3 represents N, SG1, L1, L2, L3 and L4 have the same meaning as described above.

In each of the above compounds and in the compounds below, the tert-butyl group may be replaced by cyclohexyl.

The compound may be employed, for example, in the form of its trifluoroacetic acid salt. For the reaction with the binder such as, for example, the antibody, the compound is preferably used in a 2- to 12-fold molar excess with respect to the binder.

Preferably, for coupling to a lysine residue, one of the compounds below is reacted with the lysine-containing binder such as an antibody:

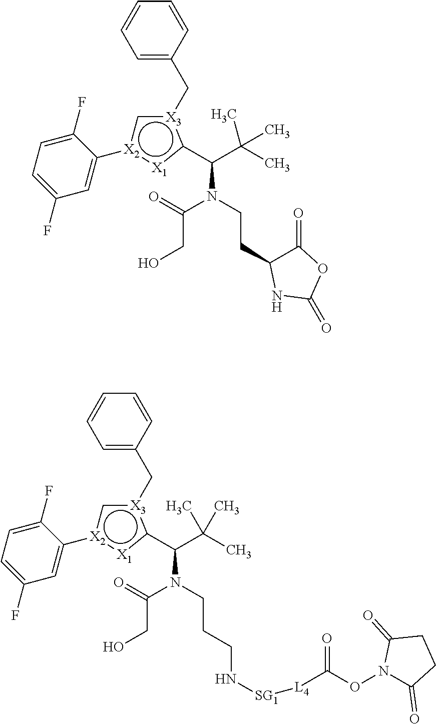

where X1 represents CH, X2 represents C and X3 represents N and LA has the same meaning as L1 and L1 has the same meaning as described above.

For an intermediate coupling to a cysteine residue, the reactions can be illustrated as follows:

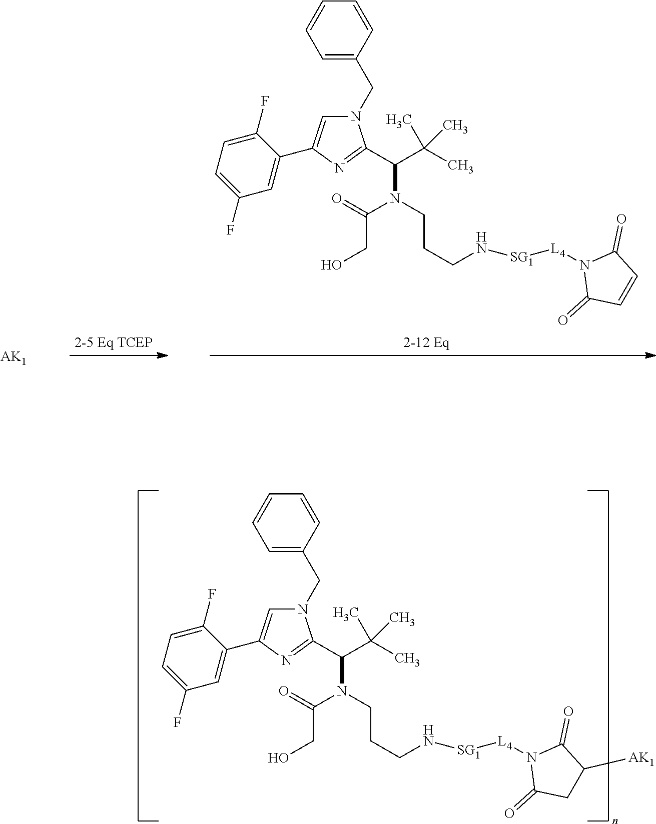

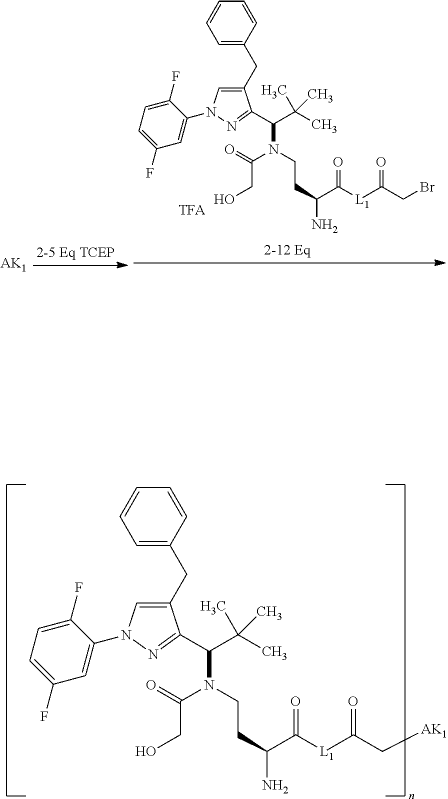

The other intermediates and other antibodies can be reacted correspondingly.

For an intermediate coupling to a lysine residue, the reaction can be illustrated as follows:

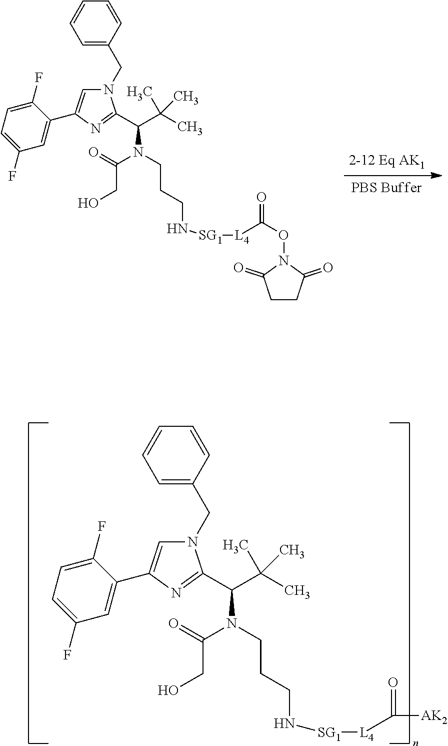

In accordance with the invention, this gives the following conjugates:

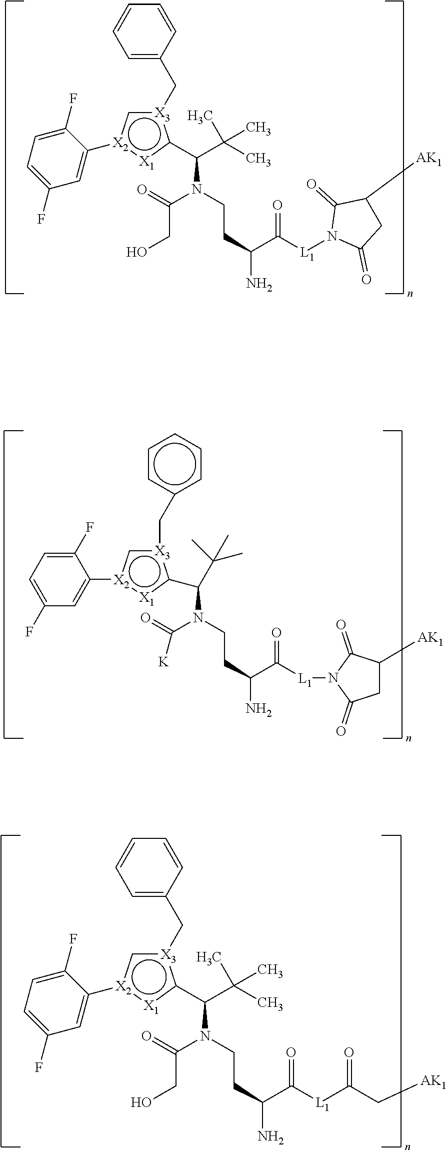

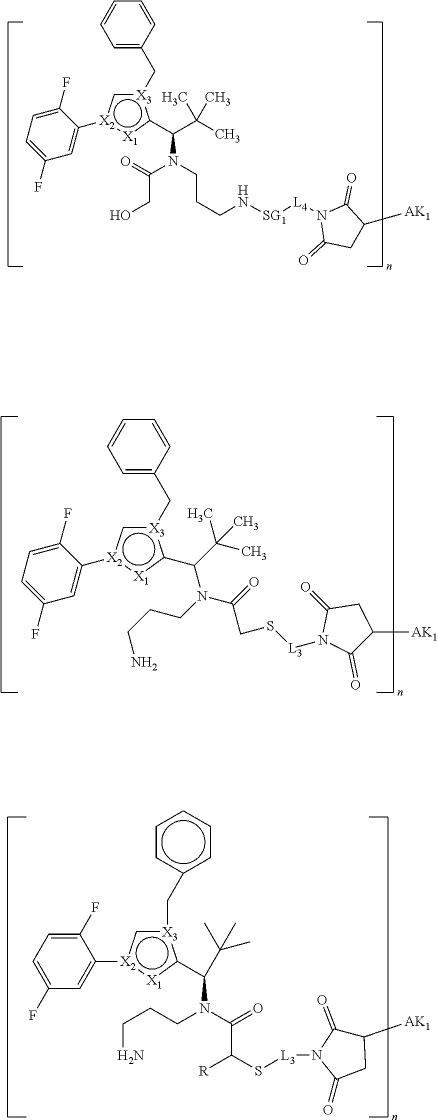

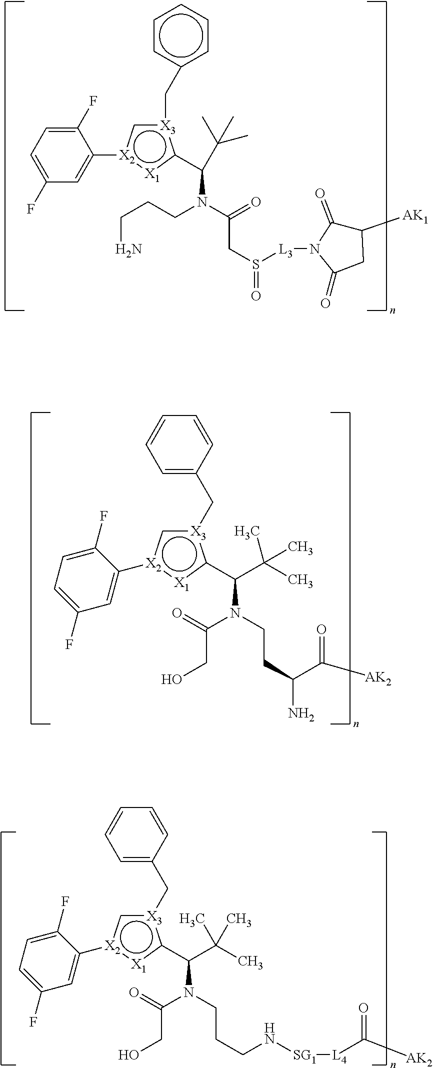

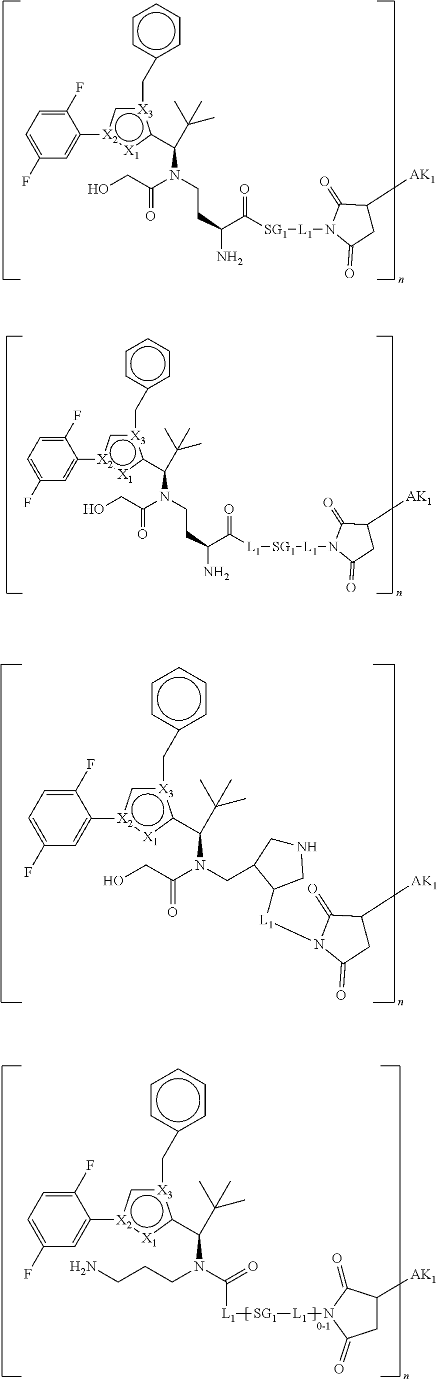

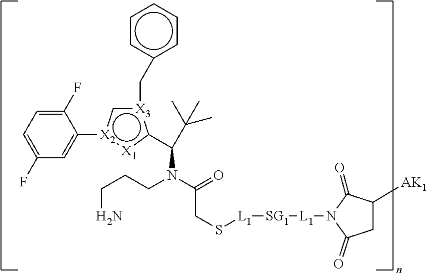

Depending on the linker, succinimide-linked ADCs may, after conjugation, be converted into the open-chain succinamides, which have an advantageous stability profile.

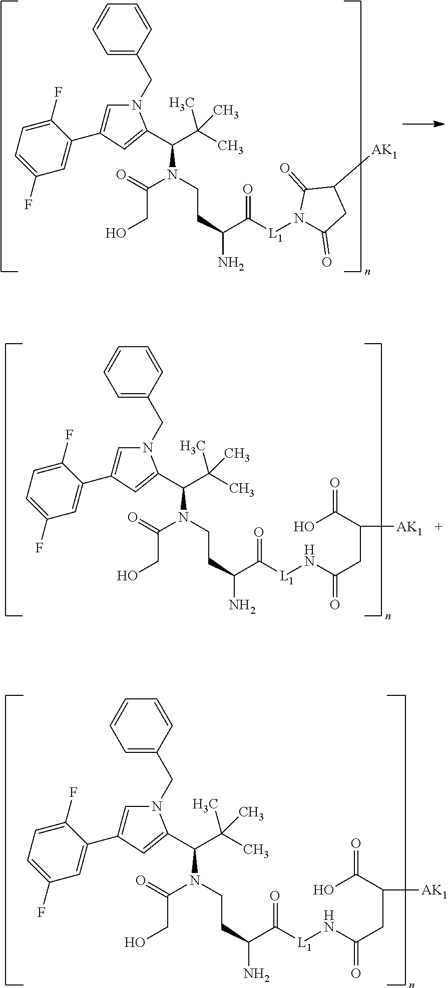

This reaction (ring opening) can be carried out at pH 7.5 to 9, preferably at pH 8, at a temperature of from 25° C. to 37° C., for example by stirring. The preferred stirring time is 8 to 30 hours.

In the above formulae, X1 represents CH, X2 represents C and X3 represents N, SG1 and L1 have the same meaning as described above and L2, L3 and L4 have the same meaning as L1; and R and K have the same meaning as described above. AK1 is a moderately agonistically or non-agonistically acting anti-TWEAKR antibody such as ITEM-4 and chimeric or humanized variants of ITEM-4 coupled via a cysteine residue, and AK2 is a moderately agonistically or non-agonistically acting anti-TWEAKR antibody such as ITEM-4 and chimeric or humanized variants of ITEM-4 coupled via a lysine residue. With particular preference, AK1 and AK2 are humanized or chimeric monoclonal ITEM-4 anti-TWEAKR antibodies. Particular preference is given to the humanized or chimeric ITEM-4 variants: TPP-7005, TPP-7006, TPP-7007, TPP-7053, TPP-7073, TPP-7074, TPP-7075 and TPP-7076.

### ITEM-4 and Humanized or Chimeric Variants Thereof

ITEM-4 is an anti-TWEAKR antibody described by Nakayama et al. (Nakayama, et al., 2003, Biochem Biophy Res Comm, 306:819-825). Humanized variants of this antibody based on CDR grafting are described by Zhou et al. (Zhou et al., 2013, J Invest Dermatol. 133(4): 1052-62) and in WO 2009/020933.

The literature also discloses various options of covalent coupling (conjugation) of organic molecules to antibodies. Preference according to the invention is given to the conjugation of the toxophores to the antibody via one or more sulphur atoms of cysteine residues of the antibody and/or via one or more NH groups of lysine residues of the antibody. However, it is also possible to bind the toxophor to the antibody via free carboxyl groups or via sugar residues of the antibody.

The antibody can be attached to the linker via a bond. Attachment of the antibody can be via a heteroatom of the binder. Heteroatoms according to the invention of the antibody which can be used for attachment are sulphur (in one embodiment via a sulphhydryl group of the antibody), oxygen (according to the invention by means of a carboxyl or hydroxyl group of the antibody) and nitrogen (in one embodiment via a primary or secondary amine group or amide group of the antibody). These heteroatoms may be present in the natural antibody or are introduced by chemical methods or methods of molecular biology. According to the invention, the attachment of the antibody to the toxophor has only a minor effect on the binding activity of the antibody with respect to the target molecule. In a preferred embodiment, the attachment has no effect on the binding activity of the antibody with respect to the target molecule.

In accordance with the present invention, the term “antibody” is to be understood in its broadest meaning and comprises immunoglobulin molecules, for example intact or modified monoclonal antibodies, polyclonal antibodies or multispecific antibodies (e.g. bispecific antibodies). An immunoglobulin molecule preferably comprises a molecule having four polypeptide chains, two heavy chains (H chains) and two light chains (L chains) which are typically linked by disulphide bridges. Each heavy chain comprises a variable domain of the heavy chain (abbreviated VH) and a constant domain of the heavy chain. The constant domain of the heavy chain may, for example, comprise three domains CH1, CH2 and CH3. Each light chain comprises a variable domain (abbreviated VL) and a constant domain. The constant domain of the light chain comprises a domain (abbreviated CL). The VH and VL domains may be subdivided further into regions having hypervariability, also referred to as complementarity determining regions (abbreviated CDR) and regions having low sequence variability (framework region, abbreviated FR). Typically, each VH and VL region is composed of three CDRs and up to four FRs. For example from the amino terminus to the carboxy terminus in the following order: FR1, CDR1, FR2, CDR2, FR3, CDR3, FR4. An antibody may be obtained from any suitable species, e.g. rabbit, llama, camel, mouse or rat. In one embodiment, the antibody is of human or murine origin. An antibody may, for example, be human, humanized or chimeric.

The term “monoclonal” antibody refers to antibodies obtained from a population of substantially homogeneous antibodies, i.e. individual antibodies of the population are identical except for naturally occurring mutations, of which there may be a small number. Monoclonal antibodies recognize a single antigenic binding site with high specificity. The term monoclonal antibody does not refer to a particular preparation process.

The term “intact” antibody refers to antibodies comprising both an antigen-binding domain and the constant domain of the light and heavy chain. The constant domain may be a naturally occurring domain or a variant thereof having a number of modified amino acid positions.

The term “modified intact” antibody refers to intact antibodies fused via their amino terminus or carboxy terminus by means of a covalent bond (e.g. a peptide bond) with a further polypeptide or protein not originating from an antibody. Furthermore, antibodies may be modified such that, at defined positions, reactive cysteines are introduced to facilitate coupling to a toxophor (see Junutula et al. Nat Biotechnol. 2008, 26(8):925-32).

The term “human” antibody refers to antibodies which can be obtained from a human or which are synthetic human antibodies. A “synthetic” human antibody is an antibody which is partially or entirely obtainable in silico from synthetic sequences based on the analysis of human antibody sequences. A human antibody can be encoded, for example, by a nucleic acid isolated from a library of antibody sequences of human origin. An example of such an antibody can be found in Soderlind et al., Nature Biotech. 2000, 18:853-856.

The term “humanized” or “chimeric” antibody describes antibodies consisting of a non-human and a human portion of the sequence. In these antibodies, part of the sequences of the human immunoglobulin (recipient) is replaced by sequence portions of a non-human immunoglobulin (donor). In many cases, the donor is a murine immunoglobulin. In the case of humanized antibodies, amino acids of the CDR of the recipient are replaced by amino acids of the donor. Sometimes, amino acids of the framework, too, are replaced by corresponding amino acids of the donor. In some cases the humanized antibody contains amino acids present neither in the recipient nor in the donor, which were introduced during the optimization of the antibody. In some cases, amino acids of the CDR of the donor are replaced by corresponding amino acids of the recipient if the latter do not contribute to the antibody binding and are potentially immunogenic. In the case of chimeric antibodies, the variable domains of the donor immunoglobulin are fused with the constant regions of a human antibody.

The term complementarity determining region (CDR) as used herein refers to those amino acids of a variable antibody domain which are required for binding to the antigen. Typically, each variable region has three CDR regions referred to as CDR1, CDR2 and CDR3. Each CDR region may embrace amino acids according to the definition of Kabat and/or amino acids of a hypervariable loop defined according to Chotia. The definition according to Kabat comprises, for example, the region from about amino acid position 24-34 (CDR1), 50-56 (CDR2) and 89-97 (CDR3) of the variable light chain and 31-35 (CDR1), 50-65 (CDR2) and 95-102 (CDR3) of the variable heavy chain (Kabat et al., Sequences of Proteins of Immunological Interest, 5th Ed. Public Health Service, National Institutes of Health, Bethesda, Md. (1991)). The definition according to Chotia comprises, for example, the region from about amino acid position 26-32 (CDR1), 50-52 (CDR2) and 91-96 (CDR3) of the variable light chain and 26-32 (CDR1), 53-55 (CDR2) and 96-101 (CDR3) of the variable heavy chain (Chothia and Lesk; J Mol Biol 196: 901-917 (1987)). In some cases, a CDR may comprise amino acids from a CDR region defined according to Kabat and Chotia.

Depending on the amino acid sequence of the constant domain of the heavy chain, antibodies may be categorized into different classes. There are five main classes of intact antibodies: IgA, IgD, IgE, IgG and IgM, and several of these can be divided into further subclasses. (Isotypes), e.g. IgG1, IgG2, IgG3, IgG4, IgA1 and IgA2. The constant domains of the heavy chain, which correspond to the different classes, are referred to as [alpha/α], [delta/δ], [epsilon/ε], [gamma/γ] and [my/μ]. Both the three-dimensional structure and the subunit structure of antibodies are known.

The term “functional fragment” or “antigen-binding antibody fragment” of an antibody/immunoglobulin is defined as a fragment of an antibody/immunoglobulin (e.g. the variable domains of an IgG) which still comprise the antigen binding domains of the antibody/immunoglobulin. The “antigen binding domain” of an antibody typically comprises one or more hypervariable regions of an antibody, for example the CDR, CDR2 and/or CDR3 region. However, the “framework” or “skeleton” region of an antibody may also play a role during binding of the antibody to the antigen. The framework region forms the skeleton of the CDRs. Preferably, the antigen binding domain comprises at least amino acids 4 to 103 of the variable light chain and amino acids 5 to 109 of the variable heavy chain, more preferably amino acids 3 to 107 of the variable light chain and 4 to 111 of the variable heavy chain, particularly preferably the complete variable light and heavy chains, i.e. amino acids 1-109 of the VL and 1 to 113 of the VH (numbering according to WO97/08320).

“Functional fragments” or “antigen-binding antibody fragments” of the invention encompass, non-conclusively, Fab, Fab′, F(ab′)2 and Fv fragments, diabodies, Single Domain Antibodies (DAbs), linear antibodies, individual chains of antibodies (single-chain Fv, abbreviated to scFv); and multispecific antibodies, such as bi and tri-specific antibodies, for example, formed from antibody fragments C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag. Antibodies other than “multispecific” or “multifunctional” antibodies are those having identical binding sites. Multispecific antibodies may be specific for different epitopes of an antigen or may be specific for epitopes of more than one antigen (see, for example, WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; or Kostelny et al., 1992, J. Immunol. 148: 1547 1553). An F(ab′)₂ or Fab molecule may be constructed such that the number of intermolecular disulphide interactions occurring between the Chi and the CL domains can be reduced or else completely prevented.

“Epitopes” refer to protein determinants capable of binding specifically to an immunoglobulin or T cell receptors. Epitopic determinants usually consist of chemically active surface groups of molecules such as amino acids or sugar side chains or combinations thereof, and usually have specific 3-dimensional structural properties and also specific charge properties.

“Functional fragments” or “antigen-binding antibody fragments” may be fused with another polypeptide or protein, not originating from an antibody, via the amino terminus or carboxyl terminus thereof, by means of a covalent bond (e.g. a peptide linkage). Furthermore, antibodies and antigen-binding fragments may be modified by introducing reactive cysteines at defined locations, in order to facilitate coupling to a toxophore (see Junutula et al. Nat Biotechnol. 2008 August; 26(8):925-32).

Polyclonal antibodies can be prepared by methods known to a person of ordinary skill in the art. Monoclonal antibodies may be prepared by methods known to a person of ordinary skill in the art (Köhler and Milstein, Nature, 256, 495-497, 1975). Human and humanized monoclonal antibodies may be prepared by methods known to a person of ordinary skill in the art (Olsson et al., Meth Enzymol. 92, 3-16 or Cabilly et al U.S. Pat. No. 4,816,567 or Boss et al U.S. Pat. No. 4,816,397).

A person of ordinary skill in the art is aware of diverse methods for preparing human antibodies and fragments thereof, such as, for example, by means of transgenic mice (N Lonberg and D Huszar, Int Rev Immunol. 1995; 13(1):65-93) or phage display technologies (Clackson et al., Nature. 1991 Aug. 15; 352(6336):624-8). Antibodies of the invention may be obtained from recombinant antibody libraries consisting for example of the amino acid sequences of a multiplicity of antibodies compiled from a large number of healthy volunteers. Antibodies may also be produced by means of known recombinant DNA technologies. The nucleic acid sequence of an antibody can be obtained by routine sequencing or is available from publically accessible databases.

An “isolated” antibody or binder has been purified to remove other constituents of the cell.

Contaminating constituents of a cell which may interfere with a diagnostic or therapeutic use are, for example, enzymes, hormones, or other peptidic or non-peptidic constituents of a cell. A preferred antibody or binder is one which has been purified to an extent of more than 95% by weight, relative to the antibody or binder (determined for example by Lowry method, UV-Vis spectroscopy or by SDS capillary gel electrophoresis). Moreover an antibody which has been purified to such an extent that it is possible to determine at least 15 amino acids of the amino terminus or of an internal amino acid sequence, or which has been purified to homogeneity, the homogeneity being determined by SDS-PAGE under reducing or non-reducing conditions (detection may be determined by means of Coomassie Blau staining or preferably by silver coloration). However, an antibody is normally prepared by one or more purification steps.

The term “specific binding” or “binds specifically” refers to an antibody or binder which binds to a predetermined antigen/target molecule. Specific binding of an antibody or binder typically describes an antibody or binder having an affinity of at least 10⁻⁷ M (as Kd value; i.e. preferably those with Kd values smaller than 10⁻⁷ M), with the antibody or binder having an at least two times higher affinity for the predetermined antigen/target molecule than for a non-specific antigen/target molecule (e.g. bovine serum albumin, or casein) which is not the predetermined antigen/target molecule or a closely related antigen/target molecule. The antibodies preferably have an affinity of at least 10⁻⁷ M (as Kd value; in other words preferably those with smaller Kd values than 10⁻⁷ M), preferably of at least 10⁻⁸ M, more preferably in the range from 10⁻⁹ M to 10⁻¹¹ M. The Kd values may be determined, for example, by means of surface plasmon resonance spectroscopy.

The antibody-drug conjugates of the invention likewise exhibit affinities in these ranges. The affinity is preferably not substantially affected by the conjugation of the drugs (in general, the affinity is reduced by less than one order of magnitude, in other words, for example, at most from 10⁻⁸ M to 10⁻⁷ M).

The antibodies used in accordance with the invention are also notable preferably for a high selectivity. A high selectivity exists when the antibody of the invention exhibits an affinity for the target protein which is better by a factor of at least 2, preferably by a factor of 5 or more preferably by a factor of 10, than for an independent other antigen, e.g. human serum albumin (the affinity may be determined, for example, by means of surface plasmon resonance spectroscopy).

Furthermore, the antibodies of the invention that are used are preferably cross-reactive. In order to be able to facilitate and better interpret preclinical studies, for example toxicological or activity studies (e.g. in xenograft mice), it is advantageous if the antibody used in accordance with the invention not only binds the human target protein but also binds the species target protein in the species used for the studies. In one embodiment the antibody used in accordance with the invention, in addition to the human target protein, is cross-reactive to the target protein of at least one further species. For toxicological and activity studies it is preferred to use species of the families of rodents, dogs and non-human primates. Preferred rodent species are mouse and rat. Preferred non-human primates are rhesus monkeys, chimpanzees and long-tailed macaques.

In one embodiment the antibody used in accordance with the invention, in addition to the human target protein, is cross-reactive to the target protein of at least one further species selected from the group of species consisting of mouse, rat and long-tailed macaque (Macaca fascicularis). Especially preferred are antibodies used in accordance with the invention which in addition to the human target protein are at least cross-reactive to the mouse target protein. Preference is given to cross-reactive antibodies whose affinity for the target protein of the further non-human species differs by a factor of not more than 50, more particularly by a factor of not more than ten, from the affinity for the human target protein.

### Antibodies Directed Against a Cancer Target Molecule

The target molecule towards which the binder, for example an antibody or an antigen-binding fragment thereof, is directed is preferably a cancer target molecule. The term “cancer target molecule” describes a target molecule which is more abundantly present on one or more cancer cell species than on non-cancer cells of the same tissue type. Preferably, the cancer target molecule is selectively present on one or more cancer cell species compared with non-cancer cells of the same tissue type, where selectively describes an at least two-fold enrichment on cancer cells compared to non-cancer cells of the same tissue type (a “selective cancer target molecule”). The use of cancer target molecules allows the selective therapy of cancer cells using the conjugates according to the invention.

Particular preference is given here to the extracellular cancer target molecule TWEAKR (SEQ ID NO: 101 (protein); SEQ ID No: 102 (DNA)). The cancer target molecule TWEAKR (human orthologue NCBI Gene ID: 51330) is also known under the name TNFRSF12A (tumour necrosis factor receptor superfamily member 12A), FN14 and CD266.

The term “anti-TWEAKR antibody” or “an antibody which specifically binds to TWEAKR” relates to an antibody which binds the cancer target molecule TWEAKR (SEQ ID NO: 101 (protein)) with an affinity which is sufficient for a diagnostic and/or therapeutic application. In one embodiment, binding of an anti-TWEAKR antibody to a protein not related to TWEAKR is less than 10% of the binding of the antibody to TWEAKR, determined, for example, by surface plasmon resonance spectroscopy. In certain embodiments, the antibody binds TWEAKR (SEQ ID NO: 101 (protein)) with a dissociation constant (KD) of ≤1 μM, ≤100 nM, ≤10 nM, ≤1 nM, ≤0.1 nM, ≤0.01 nM, or ≤0.001 nM. In certain embodiments, the anti-TWEAKR antibody binds to an epitope which is conserved between different species.

Antibodies which bind cancer target molecules may be prepared by a person of ordinary skill in the art using known processes, such as, for example, chemical synthesis or recombinant expression. Binders for cancer target molecules may be acquired commercially or may be prepared by a person of ordinary skill in the art using known processes, such as, for example, chemical synthesis or recombinant expression. Further processes for preparing antibodies or antigen-binding antibody fragments are described in WO 2007/070538 (see page 22 “Antibodies”). The person skilled in the art knows how processes such as phage display libraries (e.g. Morphosys HuCAL Gold) can be compiled and used for discovering antibodies or antigen-binding antibody fragments (see WO 2007/070538, page 24 ff and AK Example 1 on page 70, AK Example 2 on page 72). Further processes for preparing antibodies that use DNA libraries from B cells are described for example on page 26 (WO 2007/070538). Processes for humanizing antibodies are described on page 30-32 of WO2007070538 and in detail in Queen, et al., Pros. Natl. Acad. Sci. USA 86:10029-10033, 1989 or in WO 90/0786. Furthermore, processes for the recombinant expression of proteins in general and of antibodies in particular are known to the person skilled in the art (see, for example, in Berger and Kimmel (Guide to Molecular Cloning Techniques, Methods in Enzymology, Vol. 152, Academic Press, Inc.); Sambrook, et al., (Molecular Cloning: A Laboratory Manual, (Second Edition, Cold Spring Harbor Laboratory Press; Cold Spring Harbor, N.Y.; 1989) Vol. 1-3); Current Protocols in Molecular Biology, (F. M. Ausabel et al. [Eds.], Current Protocols, Green Publishing Associates, Inc./John Wiley & Sons, Inc.); Harlow et al., (Monoclonal Antibodies: A Laboratory Manual, Cold Spring Harbor Laboratory Press (19881, Paul [Ed.]); Fundamental Immunology, (Lippincott Williams & Wilkins (1998)); and Harlow, et al., (Using Antibodies: A Laboratory Manual, Cold Spring Harbor Laboratory Press (1998)). The person skilled in the art knows the corresponding vectors, promoters and signal peptides which are necessary for the expression of a protein/antibody. Commonplace processes are also described in WO 2007/070538 on pages 41-45. Processes for preparing an IgG1 antibody are described for example in WO 2007/070538 in Example 6 on page 74 ff. Processes which allow the determination of the internalization of an antibody after binding to its antigen are known to the skilled person and are described for example in WO 2007/070538 on page 80. The person skilled in the art is able to use the processes described in WO 2007/070538 that have been used for preparing carboanhydrase IX (Mn) antibodies in analogy for the preparation of antibodies with different target molecule specificity.

### Anti-TWEAKR Antibodies

According to the invention, use is made of an anti-TWEAKR antibody or an antigen-binding fragment thereof, preferably one selected from those described below or modified by suitable mutation. In addition, the person skilled in the art is familiar with antibodies binding to TWEAKR, see e.g. Nakayama, et al., 2003, Biochem Biophy Res Comm, 306:819-825; Zhou et al., 2013, J Invest Dermatol. 133(4): 1052-62: WO 2009/020933 (A2), WO2009140177 (A2) or WO2014/198817 (A1).

The invention relates in particular to conjugates with moderately agonistically or non-agonistically acting anti-TWEAKR antibodies or antigen-binding antibody fragments thereof or variants thereof derived from the antibodies ITEM-4 (Nakayama, et al., 2003, Biochem Biophy Res Comm, 306:819-825) originating from the mouse. ITEM-4 is an anti-TWEAKR antibody described by Nakayama et al. (Nakayama, et al., 2003, Biochem Biophy Res Comm, 306:819-825). The sequences of the variable regions (VH and VL) of ITEM-4 were disclosed in Zhou et al. (Zhou et al., 2013, J Invest Dermatol. 133(4):1052-62). Humanized variants of this antibody based on CDR grafting in human framework regions have been described by Zhou et al. (Zhou et al., 2013, J Invest Dermatol. 133(4): 1052-62) and in WO 2009/020933.

ITEM-4 and the humanized or chimeric variants of this antibody are, depending on the assay system used, antagonistically or moderately agonistically acting antibodies. The antagonistic action is only displayed in the presence of Tweak.

The term “moderately agonistic” or “moderately agonistically acting” anti-TWEAKR antibody refers to an antibody which binds to TWEAKR and induces the NFκB signal transduction cascade (NFκB signalling) only to a small extent when the antibody is brought into contact with a TWEAKR-expressing cell in the absence of Tweak. Induction of the NFκB signal transduction cascade (NFκB signalling) to a small extent means that an anti-TWEAKR antibody, compared to human Tweak which is employed at 200 ng/ml (100% value) in an NFκB assay, shows less than 80%, less than 50%, less than 25%, less than 20% or less than 15% of activation at an anti-TWEAKR antibody concentration employed of at most 30 μg/ml. The person skilled in the art is familiar with such NFκB assays. By way of example, one such assay is shown in the working examples.

The term “non-agonistic” or “non-agonistically acting” anti-TWEAKR antibody refers to an antibody which, in such an NFκB assay, shows 0% activity at most 30 μg/ml concentration employed. The person skilled in the art is familiar with such NFκB assays. By way of example, such an assay is shown in the working examples.

Moreover, the anti-TWEAKR antibody preferably acts antagonistically if such an assay is carried out in the presence of Tweak (the ligand).

### Generation of the Anti-TWEAKR Antibodies

Based on the publication of the sequences of the variable regions (VH and VL) of ITEM-4, in Zhou et al. (Zhou et al., 2013, J Invest Dermatol. 133(4): 1052-62) and the publication of the humanized variants of this antibody based on CDR grafting in human framework regions Zhou et al. (Zhou et al., 2013, J Invest Dermatol. 133(4): 1052-62) and WO 2009/020933, the following antibody sequences were obtained: “TPP-7007”, “TPP-7053”, “TPP-7005”, “TPP-7073”, “TPP-7075” and “TPP-7076”.

Linking the variable regions VH and VL of ITEM-4 to the constant regions (CL, CH1, CH2, CH3) of a human IgG1 of the kappa subtype, the sequences of the chimeric antibodies “TPP-7006” and “TPP-7074” are obtained.

Modification of a potential deamidation site in L-CDR1 of ITEM-4 gives the antibodies “TPP-7073”, “TPP-7074”, “TPP-7075” and “TPP-7076”.

Further humanized variants of the ITEM-4 antibody can be generated by humanization processes known in the art.

Reviews of methods for their generation can be found in Almagro and Fransson, Front. Biosci. 13:1619-1633 (2008), and further in Riechmann et al., Nature 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing resurfacing); Dall'Acqua et al., Methods 36:43-60 (2005) (describing FR shuffling); and Osbourn et al., Methods 36:61-68 (2005) and Klimka et al., Br. J. Cancer, 83:252-260 (2000) (describing the guided selection approach for FR shuffling).

### Particular Embodiments of Anti-TWEAKR Antibodies

In the present application, reference is made to the following preferred antibodies, as shown in the table below: “ITEM-4”, “TPP-7006”, “TPP-7007”, “TPP-7053”, “TPP-7005”, “TPP-7073”, “TPP-7074”, “TPP-7075” and “TPP-7076”.

ITEM-4 is a mouse IgG2b antibody described by Nakayama et al. (Nakayama, et al., 2003, Biochem Biophy Res Comm, 306:819-825). ITEM-4 can be obtained commercially from a plurality of companies (inter alia from eBioscience as 13-9018).

TPP-7006 and TPP-7074 are chimeric variants of ITEM-4 where the variable regions VH and VL are linked to the constant regions (CL, CH1, CH2, CH3) of a human IgG1 of the kappa subtype. The antibodies TPP-7007, TPP-7053, TPP-7005, TPP-7073, TPP-7075 and TPP-7076 are humanized variants of ITEM-4 as a subtype of human IgG1 kappa.

These abovementioned anti-TweakR antibodies (“ITEM-4”, “TPP-7006”, “TPP-7007”, “TPP-7053”, “TPP-7005”, “TPP-7073”, “TPP-7074”, “TPP-7075” and “TPP-7076”) also represent, independently of their non-agonistic or moderately agonistic action, preferred embodiments of the anti-TWEAKR antibody for coupling to linkers and/or toxophors according to the invention. Preferably, the anti-TWEAKR antibodies can act as antagonists in the presence of the ligand Tweak.

TABLE
Protein sequences of the antibodies:

| | SEQ ID NO: | | | | | | | | | |
|---|---|---|---|---|---|---|---|---|---|---|
| | | | | | | | | | Heavy | Light |
| | | | | | | | | | Chain | Chain |
| Antibody | VH | H-CDR1 | H-CDR2 | H-CDR3 | VL | L-CDR1 | L-CDR2 | L-CDR3 | (IgG) | (IgG) |
| TPP- | 1 | 2 | 3 | 4 | 5 | 6 | 7 | 8 | 9 | 10 |
| 7005 | | | | | | | | | | |
| TPP- | 11 | 12 | 13 | 14 | 15 | 16 | 17 | 18 | 19 | 20 |
| 7006 | | | | | | | | | | |
| TPP- | 21 | 22 | 23 | 24 | 25 | 26 | 27 | 28 | 29 | 30 |
| 7007 | | | | | | | | | | |
| TPP- | 31 | 32 | 33 | 34 | 35 | 36 | 37 | 38 | 39 | 40 |
| 7053 | | | | | | | | | | |
| TPP- | 41 | 42 | 43 | 44 | 45 | 46 | 47 | 48 | 49 | 50 |
| 7065 | | | | | | | | | | |
| TPP- | 51 | 52 | 53 | 54 | 55 | 56 | 57 | 58 | 59 | 60 |
| 7073 | | | | | | | | | | |
| TPP- | 61 | 62 | 63 | 64 | 65 | 66 | 67 | 68 | 69 | 70 |
| 7074 | | | | | | | | | | |
| TPP- | 71 | 72 | 73 | 74 | 75 | 76 | 77 | 78 | 79 | 80 |
| 7075 | | | | | | | | | | |
| TPP- | 81 | 82 | 83 | 84 | 85 | 86 | 87 | 88 | 89 | 90 |
| 7076 | | | | | | | | | | |
| TPP- | 91 | 92 | 93 | 94 | 95 | 96 | 97 | 98 | 99 | 100 |
| 7077 | | | | | | | | | | |

TPP-7005 is an antibody comprising a region of the heavy chain corresponding to SEQ ID NO: 9 and a region of the light chain corresponding to SEQ ID NO: 10.
TPP-7006 is an antibody comprising a region of the heavy chain corresponding to SEQ ID NO: 19 and a region of the light chain corresponding to SEQ ID NO: 20.
TPP-7007 is an antibody comprising a region of the heavy chain corresponding to SEQ ID NO: 29 and a region of the light chain corresponding to SEQ ID NO: 30.
TPP-7053 is an antibody comprising a region of the heavy chain corresponding to SEQ ID NO: 39 and a region of the light chain corresponding to SEQ ID NO: 40.
TPP-7065 is an antibody comprising a region of the heavy chain corresponding to SEQ ID NO: 49 and a region of the light chain corresponding to SEQ ID NO: 50.
TPP-7073 is an antibody comprising a region of the heavy chain corresponding to SEQ ID NO: 59 and a region of the light chain corresponding to SEQ ID NO: 60.
TPP-7074 is an antibody comprising a region of the heavy chain corresponding to SEQ ID NO: 69 and a region of the light chain corresponding to SEQ ID NO: 70.
TPP-7075 is an antibody comprising a region of the heavy chain corresponding to SEQ ID NO: 79 and a region of the light chain corresponding to SEQ ID NO: 80.
TPP-7076 is an antibody comprising a region of the heavy chain corresponding to SEQ ID NO: 89 and a region of the light chain corresponding to SEQ ID NO: 90.
TPP-7077 is an antibody comprising a region of the heavy chain corresponding to SEQ ID NO: 99 and a region of the light chain corresponding to SEQ ID NO: 100.
TPP-7005 is: an antibody comprising a variable region of the heavy chain corresponding to SEQ ID NO: 1 and a variable region of the light chain corresponding to SEQ ID NO: 5.
TPP-7006 is: an antibody comprising a variable region of the heavy chain corresponding to SEQ ID NO: 11 and a variable region of the light chain corresponding to SEQ ID NO: 15.
TPP-7007 is: an antibody comprising a variable region of the heavy chain corresponding to SEQ ID NO: 21 and a variable region of the light chain corresponding to SEQ ID NO: 25.
TPP-7053 is: an antibody comprising a variable region of the heavy chain corresponding to SEQ ID NO: 31 and a variable region of the light chain corresponding to SEQ ID NO: 35.
TPP-7065 is: an antibody comprising a variable region of the heavy chain corresponding to SEQ ID NO: 41 and a variable region of the light chain corresponding to SEQ ID NO: 45.
TPP-7073 is: an antibody comprising a variable region of the heavy chain corresponding to SEQ ID NO: 51 and a variable region of the light chain corresponding to SEQ ID NO: 55.
TPP-7074 is: an antibody comprising a variable region of the heavy chain corresponding to SEQ ID NO: 61 and a variable region of the light chain corresponding to SEQ ID NO: 65.
TPP-7075 is: an antibody comprising a variable region of the heavy chain corresponding to SEQ ID NO: 71 and a variable region of the light chain corresponding to SEQ ID NO: 75.
TPP-7076 is: an antibody comprising a variable region of the heavy chain corresponding to SEQ ID NO: 81 and a variable region of the light chain corresponding to SEQ ID NO: 85.
TPP-7077 is: an antibody comprising a variable region of the heavy chain corresponding to SEQ ID NO: 91 and a variable region of the light chain corresponding to SEQ ID NO: 95.

Particularly preferred embodiments of the moderately agonistically or non-agonistically acting anti-TWEAKR antibody for coupling with linkers and/or toxophores according to the invention are the antibodies below:
1. The antibody ITEM-4, a mouse IgG2b antibody described by Nakayama et al. (Nakayama, et al., 2003, Biochem Biophy Res Comm, 306:819-825).
2. An antibody or an antigen-binding fragment which binds to TWEAKR and is a chimeric or humanized variant of the antibody ITEM-4 (Nakayama, et al., 2003, Biochem Biophy Res Comm, 306:819-825).
3. An antibody or an antigen-binding fragment binding to TWEAKR, comprising:
   a variable heavy chain comprising the variable CDR1 sequence of the heavy chain, as shown in SEQ ID NO: 2, the variable CDR2 sequence of the heavy chain, as shown in SEQ ID NO: 3, and the variable CDR3 sequence of the heavy chain, as shown in SEQ ID NO: 4, and
   a variable light chain comprising the variable CDR1 sequence of the light chain, as shown in SEQ ID NO: 6, the variable CDR2 sequence of the light chain, as shown in SEQ ID NO: 7, and the variable CDR3 sequence of the light chain, as shown in SEQ ID NO: 8, or
   a variable heavy chain comprising the variable CDR1 sequence of the heavy chain, as shown in SEQ ID NO: 12, the variable CDR2 sequence of the heavy chain, as shown in SEQ ID NO: 13, and the variable CDR3 sequence of the heavy chain, as shown in SEQ ID NO: 14, and
   a variable light chain comprising the variable CDR1 sequence of the light chain, as shown in SEQ ID NO: 16, the variable CDR2 sequence of the light chain, as shown in SEQ ID NO: 17, and the variable CDR3 sequence of the light chain, as shown in SEQ ID NO: 18, or
   a variable heavy chain comprising the variable CDR1 sequence of the heavy chain, as shown in SEQ ID NO: 22, the variable CDR2 sequence of the heavy chain, as shown in SEQ ID NO: 23, and the variable CDR3 sequence of the heavy chain, as shown in SEQ ID NO: 24, and
   a variable light chain comprising the variable CDR1 sequence of the light chain, as shown in SEQ ID NO: 26, the variable CDR2 sequence of the light chain, as shown in SEQ ID NO: 27, and the variable CDR3 sequence of the light chain, as shown in SEQ ID NO: 28, or
   a variable heavy chain comprising the variable CDR1 sequence of the heavy chain, as shown in SEQ ID NO: 32, the variable CDR2 sequence of the heavy chain, as shown in SEQ ID NO: 33, and the variable CDR3 sequence of the heavy chain, as shown in SEQ ID NO: 34, and
   a variable light chain comprising the variable CDR1 sequence of the light chain, as shown in SEQ ID NO: 36, the variable CDR2 sequence of the light chain, as shown in SEQ ID NO: 37, and the variable CDR3 sequence of the light chain, as shown in SEQ ID NO: 38, or
   a variable heavy chain comprising the variable CDR1 sequence of the heavy chain, as shown in SEQ ID NO: 52, the variable CDR2 sequence of the heavy chain, as shown in SEQ ID NO: 53, and the variable CDR3 sequence of the heavy chain, as shown in SEQ ID NO: 54, and
   a variable light chain comprising the variable CDR1 sequence of the light chain, as shown in SEQ ID NO: 56, the variable CDR2 sequence of the light chain, as shown in SEQ ID NO: 57 and the variable CDR3 sequence of the light chain, as shown in SEQ ID NO: 58, or
   a variable heavy chain comprising the variable CDR1 sequence of the heavy chain, as shown in SEQ ID NO: 62, the variable CDR2 sequence of the heavy chain, as shown in SEQ ID NO: 63, and the variable CDR3 sequence of the heavy chain, as shown in SEQ ID NO: 64, and
   a variable light chain comprising the variable CDR1 sequence of the light chain, as shown in SEQ ID NO: 66, the variable CDR2 sequence of the light chain, as shown in SEQ ID NO: 67 and the variable CDR3 sequence of the light chain, as shown in SEQ ID NO: 68, or
   a variable heavy chain comprising the variable CDR1 sequence of the heavy chain, as shown in SEQ ID NO: 72, the variable CDR2 sequence of the heavy chain, as shown in SEQ ID NO: 73, and the variable CDR3 sequence of the heavy chain, as shown in SEQ ID NO: 74, and
   a variable light chain comprising the variable CDR1 sequence of the light chain, as shown in SEQ ID NO: 76, the variable CDR2 sequence of the light chain, as shown in SEQ ID NO: 77 and the variable CDR3 sequence of the light chain, as shown in SEQ ID NO: 78, or
   a variable heavy chain comprising the variable CDR1 sequence of the heavy chain, as shown in SEQ ID NO: 82, the variable CDR2 sequence of the heavy chain, as shown in SEQ ID NO: 83, and the variable CDR3 sequence of the heavy chain, as shown in SEQ ID NO: 84, and
   a variable light chain comprising the variable CDR1 sequence of the light chain, as shown in SEQ ID NO: 86, the variable CDR2 sequence of the light chain, as shown in SEQ ID NO: 87 and the variable CDR3 sequence of the light chain, as shown in SEQ ID NO: 88.
4. The antibody or an antigen-binding fragment thereof according to embodiment 3, comprising:
   a variable sequence of the heavy chain, as shown in SEQ ID NO: 1, and also a variable sequence of the light chain, as shown in SEQ ID NO: 5, or
   a variable sequence of the heavy chain, as shown in SEQ ID NO: 11, and also a variable sequence of the light chain, as shown in SEQ ID NO: 15, or
   a variable sequence of the heavy chain, as shown in SEQ ID NO: 21, and also a variable sequence of the light chain, as shown in SEQ ID NO: 25, or
   a variable sequence of the heavy chain, as shown in SEQ ID NO: 31, and also a variable sequence of the light chain, as shown in SEQ ID NO: 35, or
   a variable sequence of the heavy chain, as shown in SEQ ID NO: 51, and also a variable sequence of the light chain, as shown in SEQ ID NO: 55, or
   a variable sequence of the heavy chain, as shown in SEQ ID NO: 61, and also a variable sequence of the light chain, as shown in SEQ ID NO: 65, or
   a variable sequence of the heavy chain, as shown in SEQ ID NO: 71, and also a variable sequence of the light chain, as shown in SEQ ID NO: 75, or
   a variable sequence of the heavy chain, as shown in SEQ ID NO: 81, and also a variable sequence of the light chain, as shown in SEQ ID NO: 85.
5. The antibody according to any of the preceding embodiments which is an IgG antibody.
6. The antibody according to any of the preceding embodiments, comprising:
   a sequence of the heavy chain, as shown in SEQ ID NO: 9, and also a sequence of the light chain, as shown in SEQ ID NO: 10, or
   a sequence of the heavy chain, as shown in SEQ ID NO: 19, and also a sequence of the light chain, as shown in SEQ ID NO: 20, or
   a sequence of the heavy chain, as shown in SEQ ID NO: 29, and also a sequence of the light chain, as shown in SEQ ID NO: 30, or
   a sequence of the heavy chain, as shown in SEQ ID NO: 39, and also a sequence of the light chain, as shown in SEQ ID NO: 40, or
   a sequence of the heavy chain, as shown in SEQ ID NO: 59, and also a sequence of the light chain, as shown in SEQ ID NO: 60, or
   a sequence of the heavy chain, as shown in SEQ ID NO: 69, and also a sequence of the light chain, as shown in SEQ ID NO: 70, or
   a sequence of the heavy chain, as shown in SEQ ID NO: 79, and also a sequence of the light chain, as shown in SEQ ID NO: 80, or
   a sequence of the heavy chain, as shown in SEQ ID NO: 89, and also a sequence of the light chain, as shown in SEQ ID NO: 90.
7. The antibody according to any of the preceding embodiments, comprising: The antigen-binding fragment according to any of the preceding embodiments or an antigen-binding fragment of an antibody according to any of the preceding embodiments which is an scFv, Fab, Fab fragment or a F(ab)2 fragment.
8. The antibody or the antigen-binding fragment according to any of the preceding embodiments which is a monoclonal antibody or an antigen-binding fragment thereof.
9. The antibody or the antigen-binding fragment according to any of the preceding embodiments which is a human, humanized or chimeric antibody or an antigen-binding fragment.

Particular preference is given to the anti-TWEAKR antibodies “TPP-7005”, “TPP-7006”, “TPP-7007”, “TPP-7053”, “TPP-7073”, “TPP-7074”, “TPP-7075” and “TPP-7076”.

### Isotopes, Salts, Solvates, Isotopic Variants

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound of the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound of the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds of the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting compounds.

Preferred salts in the context of the present invention are physiologically acceptable salts of the compounds according to the invention. Also encompassed are salts which are not themselves suitable for pharmaceutical applications but can be used, for example, for isolation or purification of the compounds of the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, benzenesulphonic acid, toluenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the inventive compounds also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylpiperidine, N-methylmorpholine, arginine, lysine and 1,2-ethylene-diamine.

Designated as solvates in the context of the invention are those forms of the compounds according to the invention which form a complex in the solid or liquid state by coordination with solvent molecules. Hydrates are a specific form of the solvates in which the coordination is with water. Solvates preferred in the context of the present invention are hydrates.

The present invention additionally also encompasses prodrugs of the compounds of the invention. The term “prodrugs” in this context refers to compounds which may themselves be biologically active or inactive but are converted (for example metabolically or hydrolytically) to compounds of the invention during their residence time in the body.

### PARTICULAR EMBODIMENTS

The following embodiments are particularly preferred:

### Embodiment A

An ADC of the formula

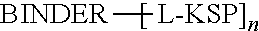

where KSP-L- represents a compound of the formula (I), (Ia), (II), (IIa), (IIb), (IIe), (IId), (IIe), (IIi), (IIj), (IIk) below or of the formula (IIf) below, the binder is a moderately agonistically or non-agonistically acting anti-TWEAKR antibody (particularly preferably a humanized or chimeric monoclonal ITEM-4 anti-TWEAKR antibody, in particular the humanized or chimeric ITEM-4 variants: TPP-7005, TPP-7006, TPP-7007, TPP-7053, TPP-7073, TPP-7074, TPP-7075 and TPP-7076), and n represents a number from 1 to 10:

Preference is given to moderately agonistically or non-agonistically acting anti-TWEAKR antibodies. The antibody is preferably a humanized or chimeric monoclonal ITEM-4 anti-TWEAKR antibody. Particular preference is given to the humanized or chimeric ITEM-4 variants: TPP-7005, TPP-7006, TPP-7007, TPP-7053, TPP-7073, TPP-7074, TPP-7075 and TPP-7076.

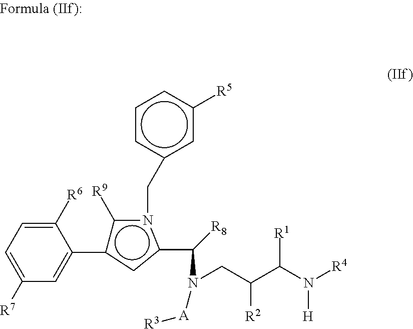

where
A represents CO (carbonyl);
R¹ represents -L-#1, H, —COOH, —CONHNH₂, —(CH₂)₁₋₃NH₂, —CONZ″(CH₂)₁₋₃ NH₂ and —CONZ″CH₂COOH, where Z″ represents H or NH₂;
R² and R⁴ represent H, or R² and R⁴ together (with formation of a pyrrolidine ring) represent —CH₂—CHR¹¹— or —CHR¹¹—CH₂—, where R¹¹ represents H or F;
R³ represents -L-#1 or a C1-10-alkyl-, which may optionally be substituted by —OH, O-alkyl, SH, S-alkyl, O—CO-alkyl, O—CO—NH-alkyl, NH—CO-alkyl, NH—CO—NH-alkyl, S(O)ₙ-alkyl, SO₂—NH— alkyl, NH-alkyl, N(alkyl)₂ or NH₂ (where alkyl is preferably C₁₋₃-alkyl);
R⁵ represents H or F;
R⁶ and R⁷ independently of one another represent H, (optionally fluorinated) C₁₋₃-alkyl, (optionally fluorinated) C₂₋₄-alkenyl, (optionally fluorinated) C₂₋₄-alkynyl, hydroxy or halogen;
R⁸ represents a branched C₁₋₅-alkyl group; and
R⁹ represents H or F,
where one of the substituents R¹ and R³ represents -L-#1, and
-L- represents the linker and #1 represents the bond to the antibody,
and salts, solvates and salts of the solvates of the ADC.

The linker is preferably a linker

§—(CO)m-L1-L2-§§

where
m represents 0 or 1;
§ represents the bond to KSP and
§§ represents the bond to the antibody, and
L2 represents

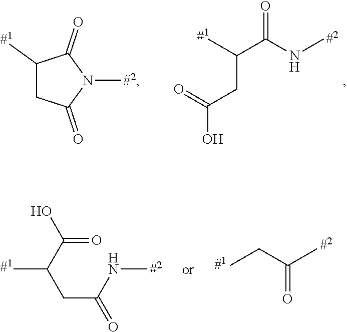

where
#¹ denotes the point of attachment to the sulphur atom of the antibody,
#² denotes the point of attachment to group L¹,
and L1 is represented by formula

#¹—(NR¹⁰)ₙ-(G1)ₒ-G2-#²

where
R¹⁰ represents H, NH₂ or C1-C3-alkyl;
G1 represents —NHCO— or

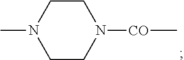

n represents 0 or 1;
o represents 0 or 1; and
G2 represents a straight-chain or branched hydrocarbon chain having 1 to 100 carbon atoms from arylene groups and/or straight-chain and/or branched and/or cyclic alkylene groups and which may be interrupted once or more than once by one or more of the groups —O—, —S—, —SO—, SO₂, —NH—, —CO—, —NHCO—, —CONH—, —NMe-, —NHNH—, —SO₂NHNH—, —CONHNH— and a 3- to 10-membered aromatic or non-aromatic heterocycle having up to 4 heteroatoms selected from the group consisting of N, O and S, or —SO— (preferably

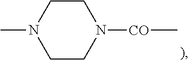

where the side chains, if present, may be substituted by —NHCONH₂, —COOH, —OH, —NH₂, NH—CNNH₂, sulphonamide, sulphone, sulphoxide or sulphonic acid.

Here, #¹ is the bond to the KSP inhibitor and #² is the bond to the coupling group to the antibody (e.g. L2).

### Embodiment B

An ADC of the formula

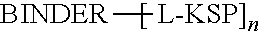

where KSP-L- represents a compound of the formula (I), (Ia), (II), (IIa), (IIb), (IIe), (IId), (IIe), (IIf), (IIi), (IIj), (IIk) below or of the formula (IIg) below, the binder is a moderately agonistically or non-agonistically acting anti-TWEAKR antibody (particularly preferably a humanized or chimeric monoclonal ITEM-4 anti-TWEAKR antibody, in particular the humanized or chimeric ITEM-4 variants: TPP-7005, TPP-7006, TPP-7007, TPP-7053, TPP-7073, TPP-7074, TPP-7075 and TPP-7076), and n represents a number from 1 to 10:

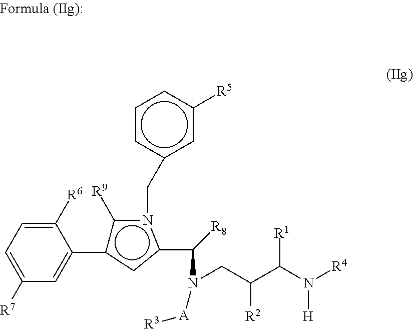

where
A represents CO (carbonyl);
R¹ represents -L-#1, H, —COOH, —CONHNH₂, —(CH₂)₁₋₃NH₂, —CONZ″(CH₂)₁₋₃ NH₂ and —CONZ″CH₂COOH, where Z″ represents H or NH₂;
R² and R⁴ represent H, or R² and R⁴ together (with formation of a pyrrolidine ring) represent —CH₂—CHR¹¹— or —CHR¹¹—CH₂—, where R¹¹ represents H;
R³ represents -L-#1 or a C₁₋₁₀-alkyl-, which may optionally be substituted by —OH, O-alkyl, SH, S-alkyl, O—CO-alkyl, O—CO—NH-alkyl, NH—CO-alkyl, NH—CO—NH-alkyl, S(O)ₙ-alkyl, SO₂—NH-alkyl, NH-alkyl, N(alkyl)₂ or NH₂ (where alkyl is preferably C₁₋₃-alkyl);
R⁵ represents H or F;
R⁶ and R⁷ independently of one another represent H, (optionally fluorinated) C₁₋₃-alkyl, (optionally fluorinated) C₂₋₄-alkenyl, (optionally fluorinated) C₂₋₄-alkynyl, hydroxy or halogen;
R⁸ represents a branched C₁₋₅-alkyl group; and
R⁹ represents H or F,
where one of the substituents R¹ and R³ represents -L-#1, and
-L- represents the linker and #1 represents the bond to the antibody,
where -L- is represented by

§—(CO)m-L1-L2-§§

where
m represents 0 or 1;
§ represents the bond to KSP and
§§ represents the bond to the antibody, and
L2 represents

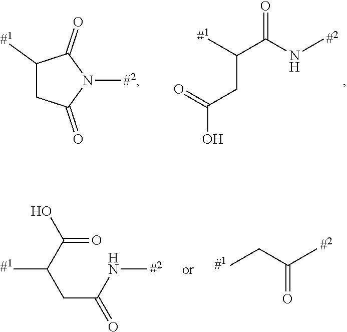

where
#¹ denotes the point of attachment to the sulphur atom of the antibody,
#² denotes the point of attachment to group L¹,
and L1 is represented by formula

#¹—(NR¹⁰)ₙ-(G1)ₒ-G2-#²

where
R¹⁰ represents H, NH₂ or C1-C3-alkyl;
G1 represents —NHCO— or

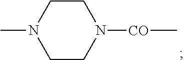

n represents 0 or 1;
o represents 0 or 1; and
G2 represents a straight-chain or branched hydrocarbon chain having 1 to 100 carbon atoms from arylene groups and/or straight-chain and/or branched and/or cyclic alkylene groups and which may be interrupted once or more than once by one or more of the groups —O—, —S—, —SO—, SO₂, —NH—, —CO—, —NHCO—, —CONH—, —NMe-, —NHNH—, —SO₂NHNH—, —CONHNH— and a 3- to 10-membered aromatic or non-aromatic heterocycle having up to 4 heteroatoms selected from the group consisting of N, O and S, or —SO— (preferably

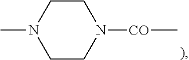

where the side chains, if present, may be substituted by —NHCONH₂, —COOH, —OH, —NH₂, NH—CNNH₂, sulphonamide, sulphone, sulphoxide or sulphonic acid,
#¹ is the bond to the KSP inhibitor and #² is the bond to the coupling group to the antibody (e.g. L2),
and salts, solvates and salts of the solvates of the ADC.

### Embodiment C

An ADC of the formula

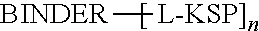

where KSP-L- represents a compound of the formula (II), (IIa), (IIb), (IIe), (IId), (IIe), (IIf), (IIg); (IIi), (IIj), (IIk) below or of the formula (IIh) below, the binder is a moderately agonistically or non-agonistically acting anti-TWEAKR antibody (particularly preferably a humanized or chimeric monoclonal ITEM-4 anti-TWEAKR antibody, in particular the humanized or chimeric ITEM-4 variants: TPP-7005, TPP-7006, TPP-7007, TPP-7053, TPP-7073, TPP-7074, TPP-7075 and TPP-7076), and n represents a number from 1 to 10:

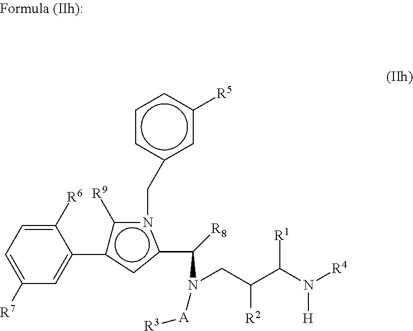

where
A represents CO (carbonyl);
R¹ represents -L-#1;
R² and R⁴ represent H, or R² and R⁴ together (with formation of a pyrrolidine ring) represent —CH₂—CHR¹¹— or —CHR¹¹—CH₂—, where R¹¹ represents H;
R³ represents C₁₋₁₀-alkyl-, which may optionally be substituted by —OH, O-alkyl, SH, S-alkyl, O—CO-alkyl, O—CO—NH-alkyl, NH—CO-alkyl, NH—CO—NH-alkyl, S(O)ₙ-alkyl, SO₂—NH-alkyl, NH-alkyl, N(alkyl)₂ or NH₂ (where alkyl is preferably C₁₋₃-alkyl), or -MOD;
where -MOD represents —(NR¹⁰)ₙ-(G1)ₒ-G2-G3, where
R¹⁰ represents H or C₁-C₃-alkyl;
G1 represents —NHCO— or —CONH— (where, if G1 represents —NHCO— or

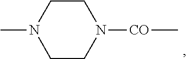

R¹⁰ does not represent NH₂);
n represents 0 or 1;
o represents 0 or 1; and
G2 represents a straight-chain or branched hydrocarbon group which has 1 to 10 carbon atoms and which may be interrupted once or more than once by one or more of the groups —O—, —S—, —SO—, SO₂, -NRy-, -NRyCO—, CONRy-, -NRyNRy-, —SO₂NRyNRy-, -CONRyNRy- (where R^{y} represents H, phenyl, C1-C10-alkyl, C2-C10-alkenyl or C2-C10-alkynyl, each of which may be substituted by NHCONH₂, —COOH, —OH, —NH₂, NH—CNNH₂, sulphonamide, sulphone, sulphoxide or sulphonic acid), —CO—, —CR^{x}═N—O— (where Rx represents H, C1-C3-alkyl or phenyl), where the hydrocarbon chain including any side chains may be substituted by —NHCONH₂, —COOH, —OH, —NH₂, NH—CNNH₂, sulphonamide, sulphone, sulphoxide or sulphonic acid, G3 represents —H or —COOH, where the group -MOD preferably has at least one group —COOH;
R⁵ represents H or F;
R⁶ and R⁷ independently of one another represent H, (optionally fluorinated) C₁₋₃-alkyl, (optionally fluorinated) C₂₋₄-alkenyl, (optionally fluorinated) C₂₋₄-alkynyl, hydroxy or halogen;
R⁸ represents a branched C₁₋₅-alkyl group; and
R⁹ represents H or F,
where -L- represents the linker and #1 represents the bond to the antibody,
where -L- is represented by

§—(CO)m-L1-L2-§§

where
m represents 0 or 1;
§ represents the bond to KSP and
§§ represents the bond to the antibody, and
L2 represents

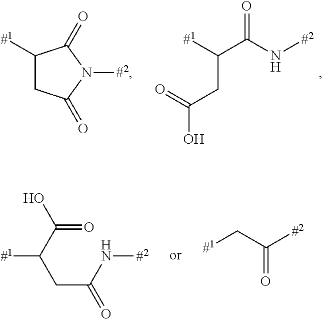

where
#¹ denotes the point of attachment to the sulphur atom of the antibody,
#² denotes the point of attachment to group L¹,
and L1 is represented by formula

#¹—(NR¹⁰)ₙ-(G1)ₒ-G2-#²

where
R¹⁰ represents H, NH₂ or C1-C3-alkyl;
G1 represents —NHCO— or

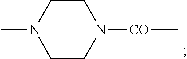

n represents 0 or 1;
o represents 0 or 1; and
G2 represents a straight-chain or branched hydrocarbon chain having 1 to 100 carbon atoms from arylene groups and/or straight-chain and/or branched and/or cyclic alkylene groups and which may be interrupted once or more than once by one or more of the groups —O—, —S—, —SO—, SO₂, —NH—, —CO—, —NHCO—, —CONH—, —NMe-, —NHNH—, —SO₂NHNH—, —CONHNH—, —CR^{x}═N—O— (where Rx represents H, C1-C3-alkyl or phenyl) and a 3- to 10-membered aromatic or non-aromatic heterocycle having up to 4 heteroatoms selected from the group consisting of N, O and S, —SO— or —SO₂— (preferably

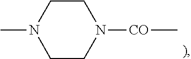

where the hydrocarbon chain including the side chains, if present, may be substituted by —NHCONH₂, —COOH, —OH, —NH₂, NH—CNNH₂, sulphonamide, sulphone, sulphoxide or sulphonic acid,
#¹ is the bond to the KSP inhibitor and #² is the bond to the coupling group to the antibody (e.g. L2),
and salts, solvates, salts of the solvates and epimers of the ADC.

### Embodiment D

The invention also provides binder/active compound conjugates of the general formula below:

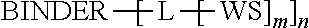

where BINDER represents the moderately agonistically or non-agonistically acting anti-TWEAKR antibody (particularly preferably a humanized or chimeric monoclonal ITEM-4 anti-TWEAKR antibody, in particular the humanized or chimeric ITEM-4 variants: TPP-7005, TPP-7006, TPP-7007, TPP-7053, TPP-7073, TPP-7074, TPP-7075 and TPP-7076), L represents the linker, WS represents the active compound, preferably a KSP inhibitor such as, for example, a KSP inhibitor according to the invention of one of the formulae (I), (Ia), (II), (IIa), (IIb), (IIe), (IId), (IIe), (IIf), (IIg), (IIh) (IIi), m represents a number from 1 to 2, preferably 1, and n represents a number from 1 to 50, preferably from 1.2 to 20 and particularly preferably from 2 to 8, where L has one of the structures below. Here, m represents the number of active compound molecules per linker and n a mean of the number of active compound/linker conjugates per BINDER. The sum of all WS present in a conjugate molecule is therefore the product of m and n.

WS is an active compound which has local or systemic therapeutic action in animals, preferably in humans. These active compounds generally have a molecular weight below 5 kDa, preferably below 1.5 kDa. Preferred active compounds are vinca alkaloids, auristatins, tubulysins, duocarmycins, kinase inhibitors, MEK inhibitors and KSP inhibitors.

Here, L represents one of the formulae A3 and A4 below

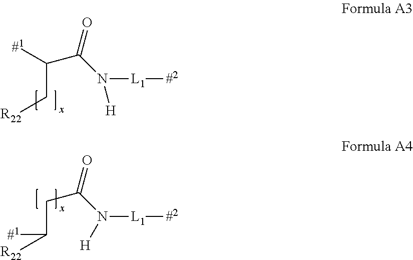

where #¹ denotes the point of attachment to the sulphur atom of the binder, #² denotes the point of attachment to the active compound, x represents 1 or 2, and R22 represents COOH, COOR, COR (where R in each case represents C1-3-alkyl), CONH₂, Br, preferably COOH.
L1 has the same meaning as above. Preferably, -L1-#2 is represented by the formula below:

#³—(NR¹⁰)ₙ-(G1)ₒ-G2-#²

where
#3 denotes the point of attachment to the nitrogen atom,
R¹⁰ represents H, NH₂ or C₁-C₃-alkyl;
G1 represents —NHCO—, —CONH— or

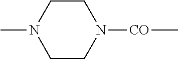

(where, if G1 represents NHCO or

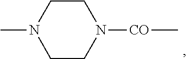

R10 does not represent NH₂),
n represents 0 or 1;
o represents 0 or 1; and
G2 represents a straight-chain or branched hydrocarbon chain which has 1 to 100 carbon atoms from arylene groups and/or straight-chain and/or branched and/or cyclic alkylene groups and which may be interrupted once or more than once by one or more of the groups —O—, —S—, —SO—, SO₂, -NRy-, -NRyCO—, —C(NH)NRy-, CONRy-, -NRyNRy-, —SO₂NRyNRy-, -CONRyNRy- (where R^{y} represents H, phenyl, C1-C10-alkyl, C2-C10-alkenyl or C2-C10-alkynyl, each of which may be substituted by NHCONH2, —COOH, —OH, —NH₂, NH—CNNH₂, sulphonamide, sulphone, sulphoxide or sulphonic acid), —CO—, —CR^{x}═N-G- (where R^{x} represents H, C1-C3-alkyl or phenyl) and/or a 3- to 10-membered aromatic or non-aromatic heterocycle having up to 4 heteroatoms selected from the group consisting of N, O and S, —SO— or —SO₂— (preferably

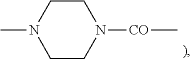

where the hydrocarbon chain including any side chains may be substituted by —NHCONH₂, —COOH, —OH, —NH₂, NH—CNNH₂, sulphonamide, sulphone, sulphoxide or sulphonic acid.

Further interrupting groups in G2 are preferably

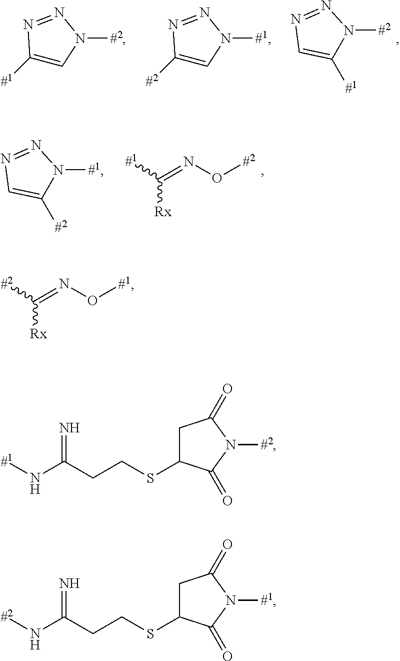

where R^{x} represents H, C₁-C₃-alkyl or phenyl.

In the conjugate according to the invention or in a mixture of the conjugates according to the invention, the bonds to a cysteine residue of the antibody are present, to an extent of preferably more than 80%, particularly preferably more than 90% (in each case based on the total number of bonds of the linker to the antibody) as one of the two structures of the formula A3 or A4.

The conjugates with the linkers of formula A3 or A4 can be obtained by coupling the antibodies to the appropriate bromine derivatives of the formulae A3′ and A4′, respectively, below:

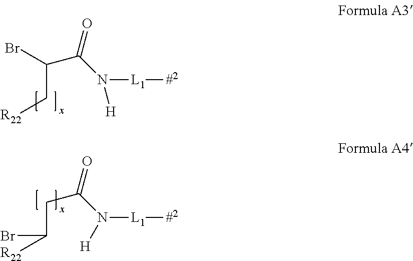

These bromine derivatives of the formula A3′ or A4′ can be obtained by reacting R₂₂CH₂CHBrCOOH or R₂₂CHBrCH₂COOH with an amine group of the binder, as illustrated in an exemplary manner in Schemes 30 to 32 below.

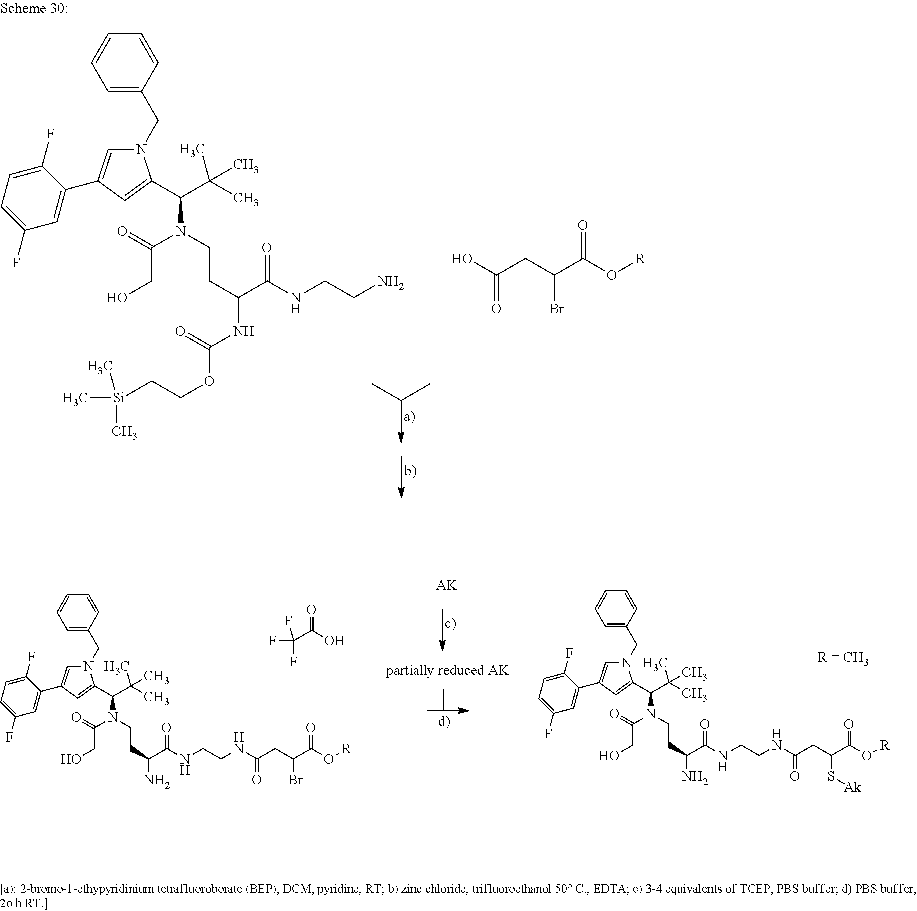

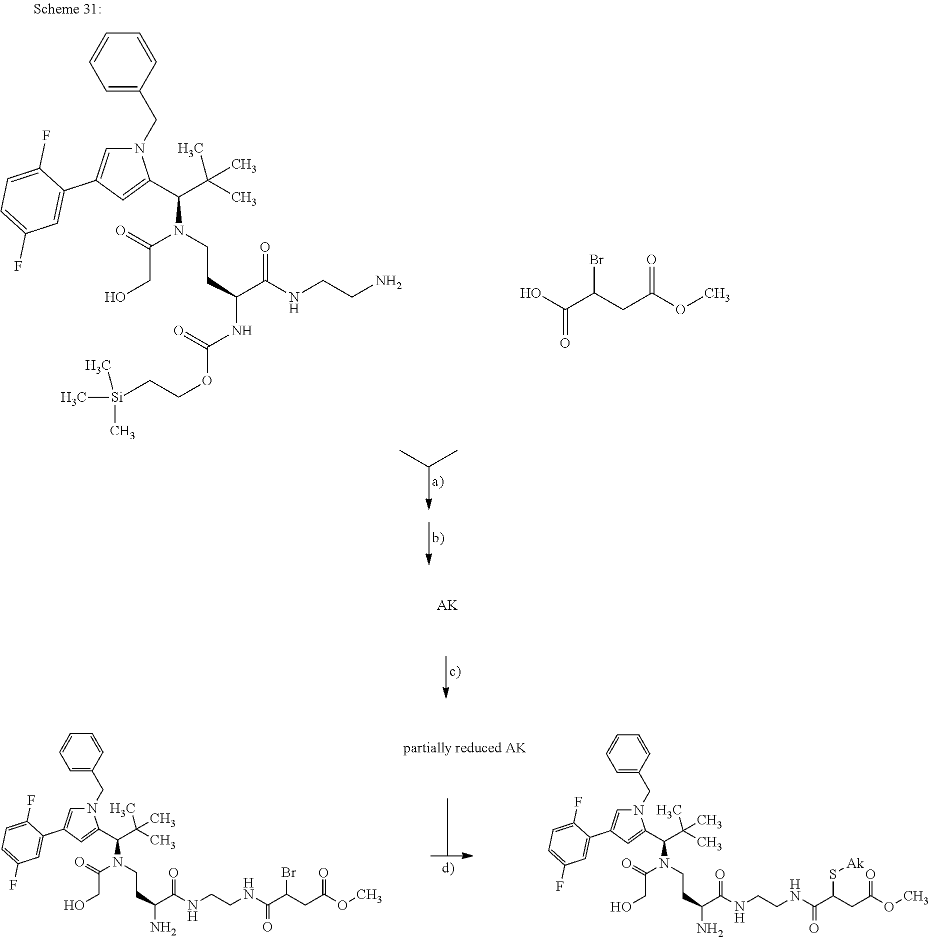

[a): 2-bromo-1-ethylpyridinium tetrafluoroborate (BEP), DCM, pyridine, RT; b) zinc chloride, trifluoroethanol, 50° C., EDTA; c) 3-4 equivalents of TCEP, PBS buffer; d) PBS buffer, 20 h RT.]

### Embodiment E

The invention also provides binder/active compound conjugates of the general formula below:

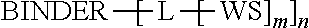

where BINDER represents the moderately agonistically or non-agonistically acting anti-TWEAKR antibody (particularly preferably a humanized or chimeric monoclonal ITEM-4 anti-TWEAKR antibody, in particular the humanized or chimeric ITEM-4 variants: TPP-7005, TPP-7006, TPP-7007, TPP-7053, TPP-7073, TPP-7074, TPP-7075 and TPP-7076), L represents the linker, WS represents the active compound, preferably a KSP inhibitor such as, for example, a KSP inhibitor according to the invention of one of the formulae (I), (Ia), (II), or (IIa), m represents a number from 1 to 2, preferably 1, and n represents a number from 1 to 50, preferably from 1.2 to 20 and particularly preferably from 2 to 8, where L has one of the structures below. Here, m represents the number of active compound molecules per linker and n a mean of the number of active compound/linker conjugates per BINDER. The sum of all WS present in a conjugate molecule is therefore the product of m and n.

Here, L represents:

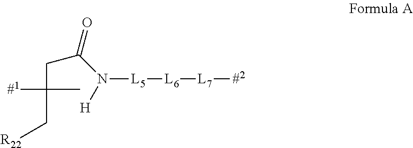

where #¹ denotes the point of attachment to the sulphur atom of the antibody, #² denotes the point of attachment to the active compound and R²² represents COOH, COOR, COR (where R in each case represents C1-3-alkyl), CONH₂, Br, preferably COOH; The link to the sulphur atom of the binder may thus have one of the structures below:

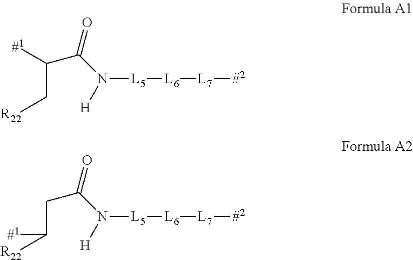

In the case of antibody drug conjugates containing more than one active compound molecule WS per antibody drug conjugate, both structures according to the formulae A1 and/or A2 may be present in an antibody drug conjugate. Since the antibody drug conjugates according to the invention may be mixtures of different antibody drug conjugates, it is also possible for this mixture to comprise both antibody drug conjugates of formula A1 or formula A2 and those of formula A1 and A2.

L₅ is a group selected from —(CH₂)ₘ—(CHRS)ₙ—(OCH₂CH₂)ₒ—(X)ₚ—(CH₂)_{q}—, where m, n, o, p and q independently of one another have the following values: m=0-10; n=0 or 1; o=0-10; p=0 or 1; and q=0-10, where m+n+o=1-15, preferably 1-6. X represents a 5- or 6-membered aromatic or nonaromatic hetero- or homocycle, preferably —C₆H₄— or —C₆H₁₀—. RS represents an acid group, preferably —COOH or SG₃H.

L₆ is a group selected from —CONH—, —OCONH—, —NHCO—, —NHCOO—,

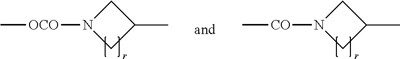

where r is 1, 2 or 3.

L₇ is a single bond or a group selected from a straight-chain or branched hydrocarbon chain which has 1 to 100 (preferably 1 to 10) carbon atoms from arylene groups and/or straight-chain and/or branched and/or cyclic alkylene groups and which may be interrupted once or more than once by one or more of the groups —O—, —S—, —SO—, SO₂, -NRy-, -NRyCO—, —C(NH)NRy-, CONRy-, -NRyNRy-, —SO₂NRyNRy-, -CONRyNRy- (where R^{y} represents H, phenyl, C1-C10-alkyl, C2-C10-alkenyl or C2-C10-alkynyl, each of which may be substituted by NHCONH₂, —COOH, —OH, —NH₂, NH—CNNH₂, sulphonamide, sulphone, sulphoxide or sulphonic acid), —CO—, —CR^{x}═N—O— (where Rx represents H, C1-C3-alkyl or phenyl) and/or a 3- to 10-membered, preferably 5- to 10-membered aromatic or non-aromatic heterocycle having up to 4 heteroatoms selected from the group consisting of N, O and S, —SO— or —SO₂— (preferably

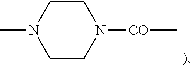

where the hydrocarbon chain including any side chains may be substituted by —NHCONH₂, —COOH, —OH, —NH₂, NH—CNNH₂, sulphonamide, sulphone, sulphoxide or sulphonic acid.

L₅ is preferably a group —(CH₂)ₘ—(CHRS)ₙ—(OCH₂CH₂)ₒ—(X)ₚ—(CH₂)_{q}— where m=1-3, n=0, o=0-7, p=0 and q=0 or 1. Particular preference is given to a group —(CH₂)ₘ—(CHRS)ₙ—(OCH₂CH₂)ₒ—(X)ₚ—(CH₂)_{q}— where m=1 or 2, n=0, o=0 or 1, p=0 and q=0 or 1.

L₆ is preferably a group selected from —CONH— and —NHCO—.

L₇ is preferably a single bond or —[(CH₂)ₓ—(X⁴)_{y}]w-(CH₂)_{z}—,

where
w=0 to 20;
x=0 to 5;
y=0 or 1;
z=1 to 5; and
X⁴ represents —O—, —CONH—, —NHCO— or

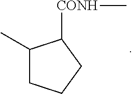

Particularly preferably, L₇ is a single bond or a group —[(CH₂)ₓ—NHCO—)], where x=1 to 5.

Particularly preferably, -L₅-L₆-L₇-represents —(CH₂)ₘ—(CHRS)ₙ—(OCH₂CH₂)ₒ—(X)ₚ—(CH₂)_{q}—NHCO—[(CH₂)ₓ—NHCO—)], where m=1 or 2, n=0, o=0 or 1, p=0, and q=0 or 1, and x=1-5.

However, it is also possible that these two structures are jointly present in the conjugate according to the invention.

According to the invention, these antibody drug conjugates can be prepared from the compounds of the formula

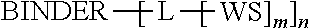

where L has the formula A′ below:

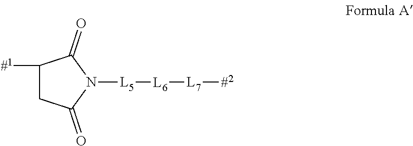

Preferably, the conversion of A′ into A is carried out by stirring in a pH buffer having a pH of from 7.5 to 8.5, preferably 8, at a temperature below 37° C., preferably from 10 to 25° C., over a period of up to 40 hours, preferably 1 to 15 hours.

### Embodiment I

An antibody drug conjugate of the formula

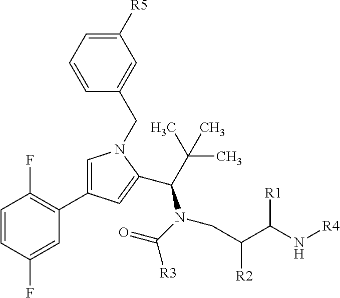

where
R2, R4 and R5 represent H;
R3 represents —CH₂OH;
R1 represents -L1-L2-BINDER, where
L1 represents

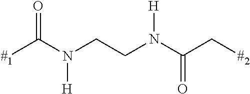

where #2 represents the attachment to L2 and #1 represents the attachment to the other attachment;
and L2 represents one or both of the structures of the formulae A5 and A6 below:

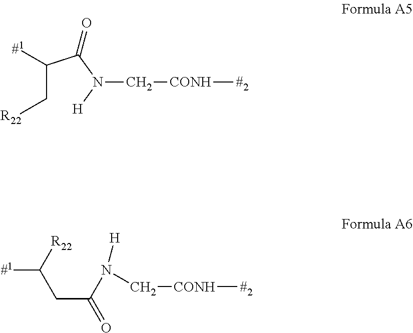

where
#¹ denotes the point of attachment to the sulphur atom of the antibody,
#² denotes the point of attachment to group L¹, and
   R²² represents COOH, COOR, COR, CONHR (where R in each case represents C1-3-alkyl), CONH₂, preferably COOH.

In a conjugate according to the invention or in a mixture of the conjugates according to the invention, the bonds to a cysteine residue of the antibody are present, to an extent of preferably more than 80%, particularly preferably more than 90% (in each case based on the total number of bonds of the linker to the antibody), particularly preferably as one of the two structures of the formula A5 or A6: Here, the structures of the formula A5 or A6 are generally present together, preferably in a ratio of from 60:40 to 40:60, based on the number of bonds to the antibody. The remaining bonds are then present as the structure

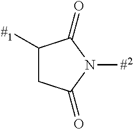

The antibody is preferably a humanized or chimeric monoclonal ITEM-4 anti-TWEAKR antibody. Particular preference is given to the humanized or chimeric ITEM-4 variants: TPP-7005, TPP-7006, TPP-7007, TPP-7053, TPP-7073, TPP-7074, TPP-7075 and TPP-7076.

### Specific Embodiments

The following particularly preferred antibody conjugates according to one of the formulae below are provided, where n is a number from 1 to 20 and AK1 (as well as AK1a, AK1b, etc.) and AK2 (as well as AK2a, AK2b, etc.) are antibodies. AK1 is an antibody attached via cysteine, AK2 is an antibody attached via lysine. The antibody (AK1 or AK2) in one of the following formulae is preferably a humanized or chimeric monoclonal ITEM-4 anti-TWEAKR antibody. Particular preference is given to the humanized or chimeric ITEM-4 variants: TPP-7005, TPP-7006, TPP-7007, TPP-7053, TPP-7073, TPP-7074, TPP-7075 and TPP-7076.

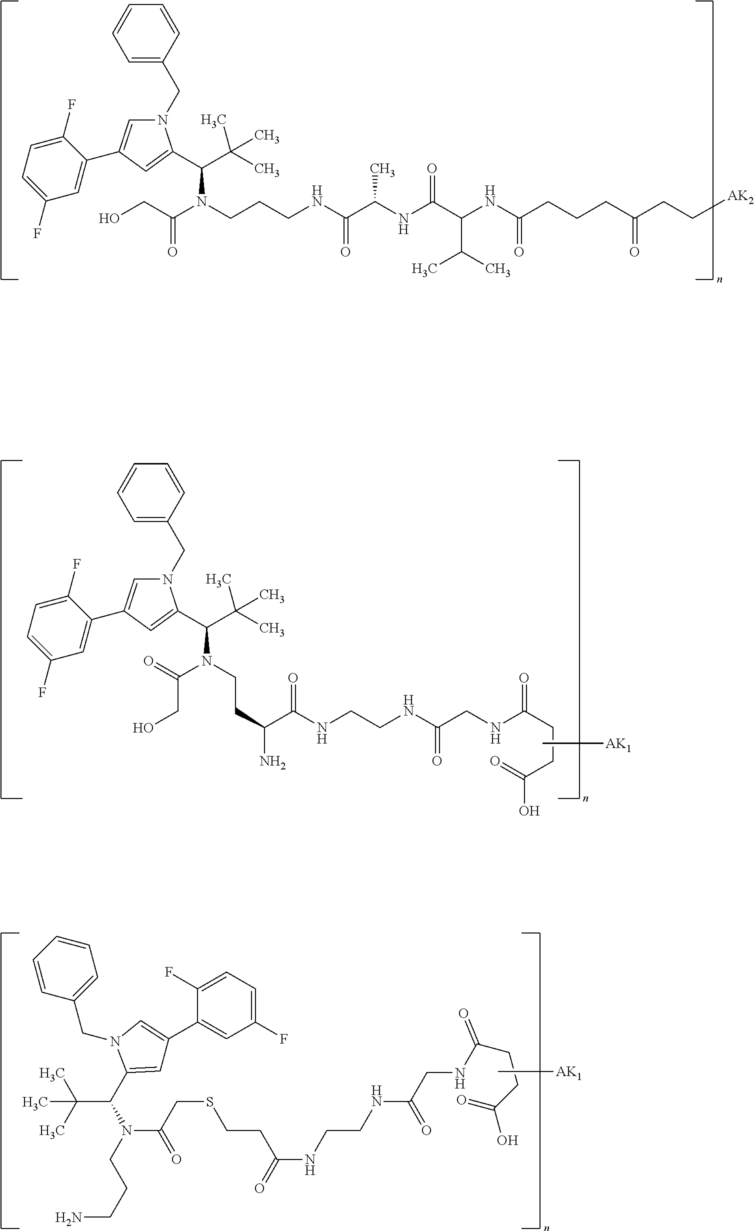

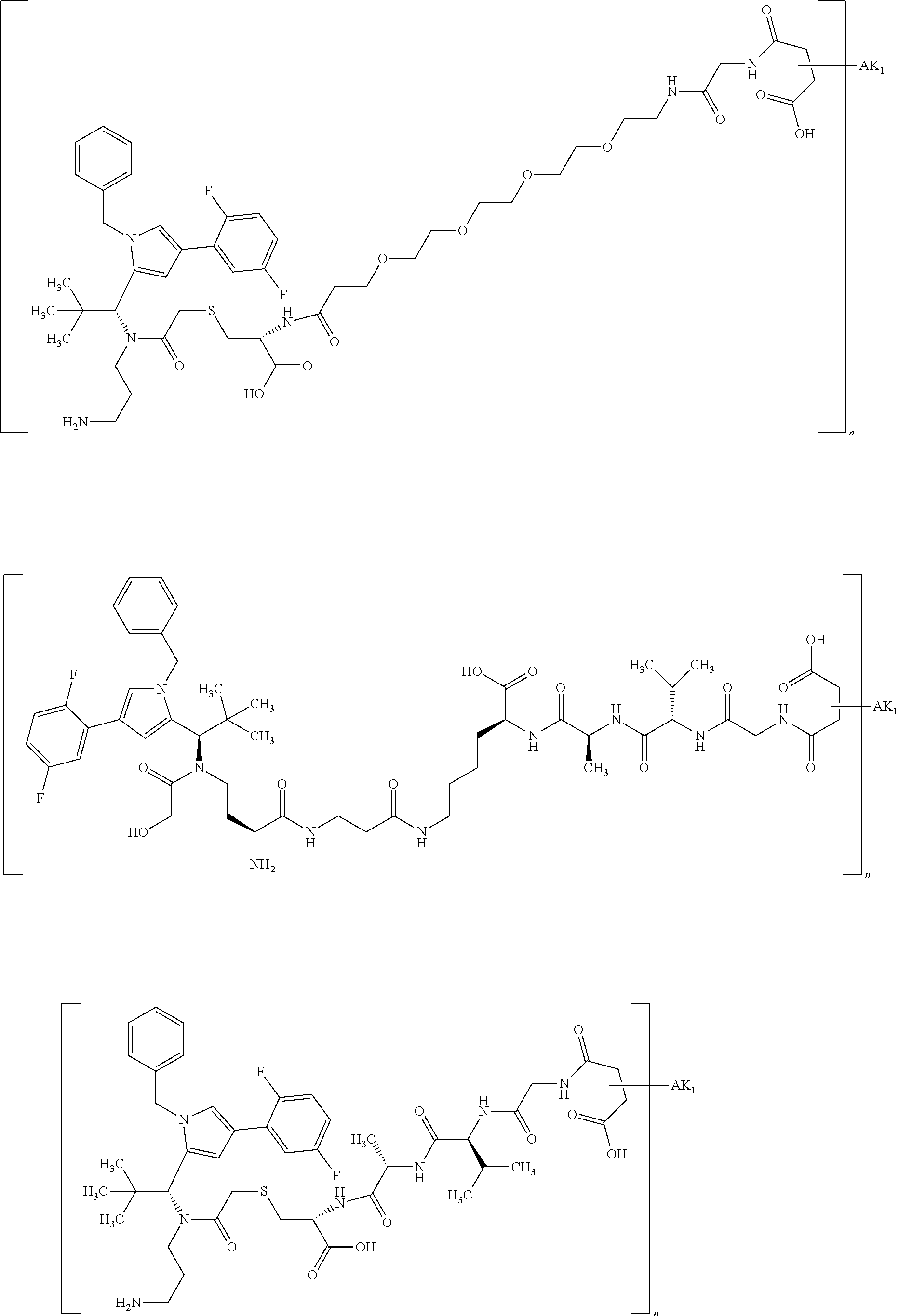

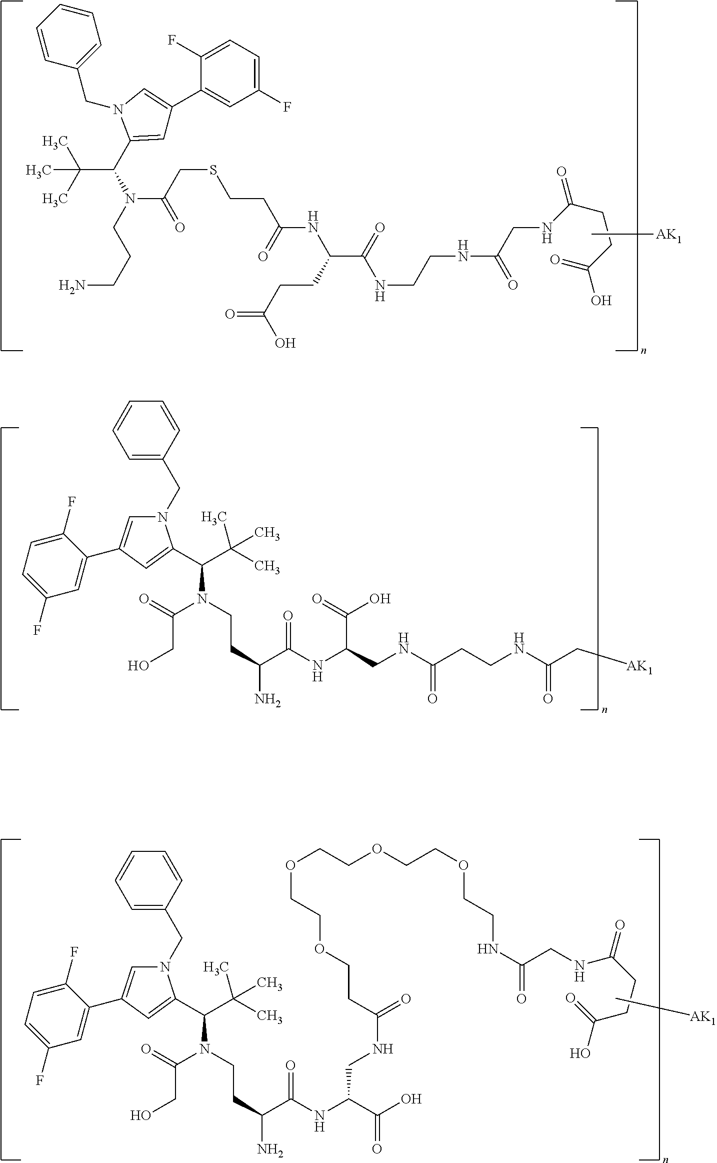

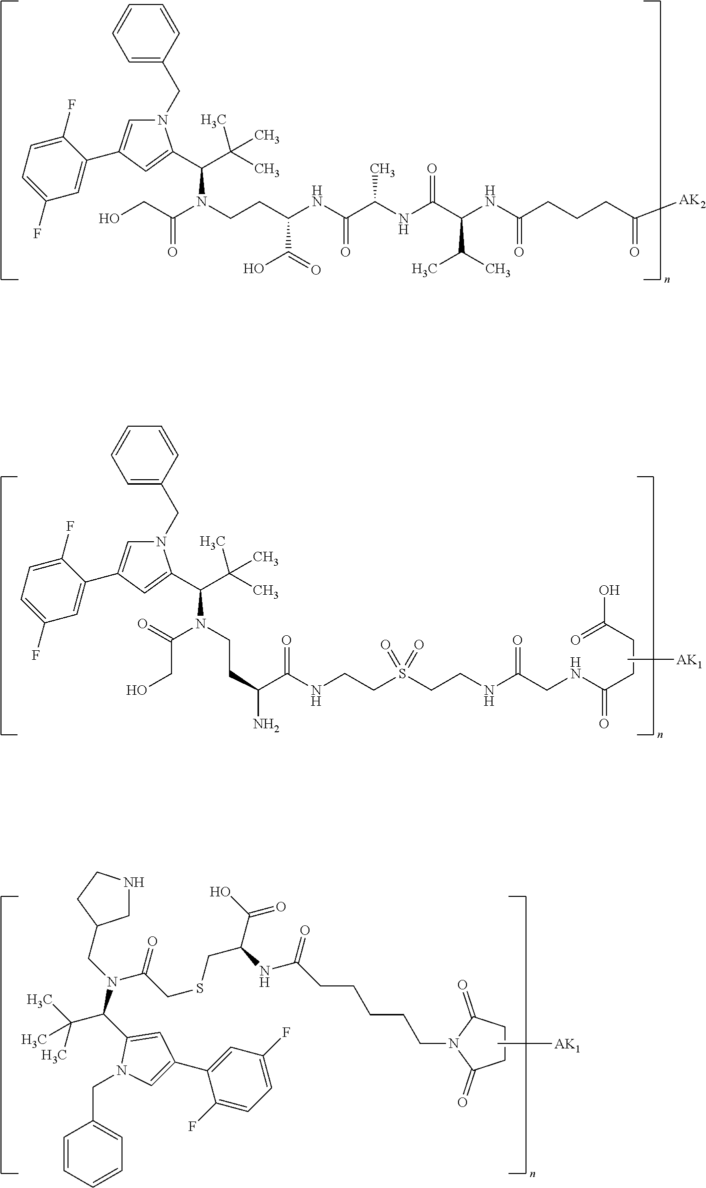

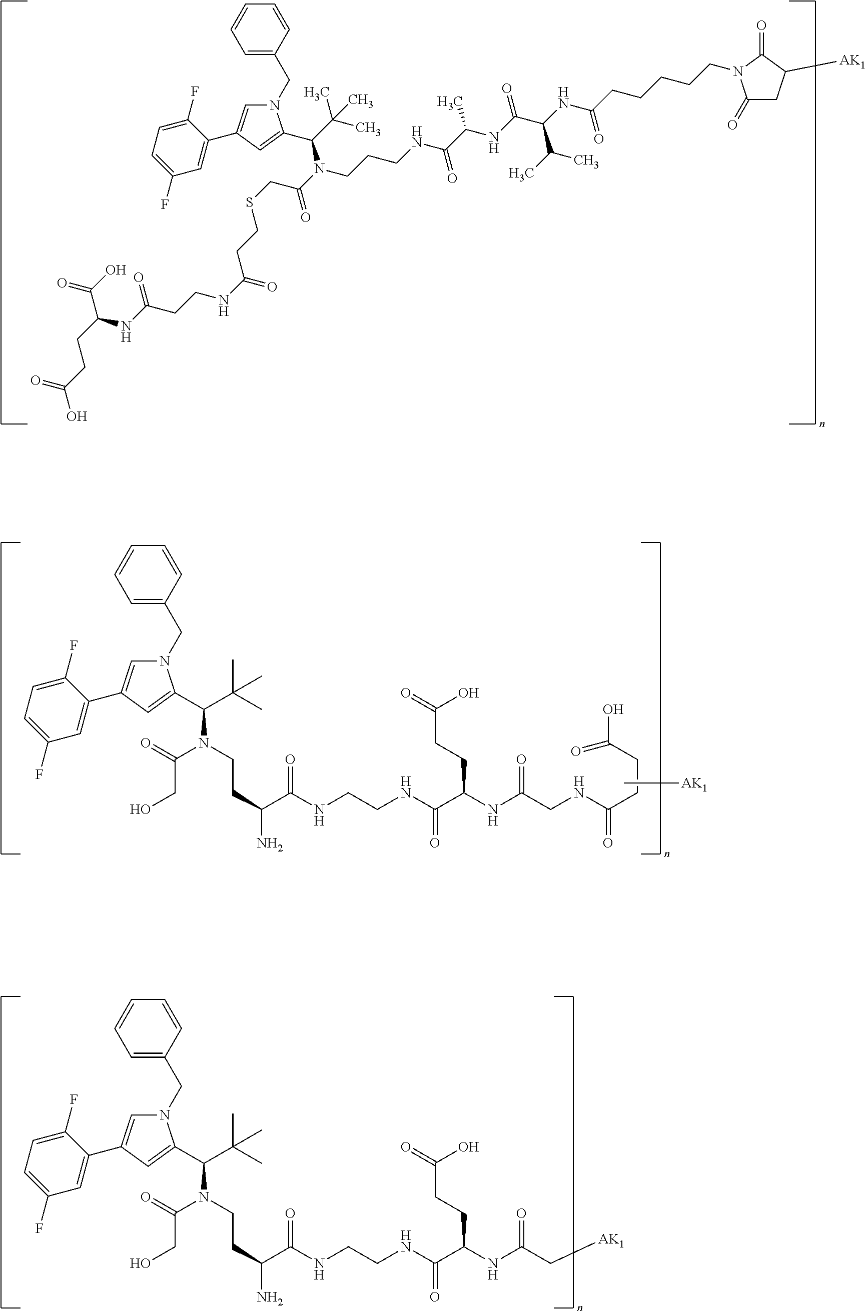

### Further Conjugates

Further conjugates may have one of the following formulae:

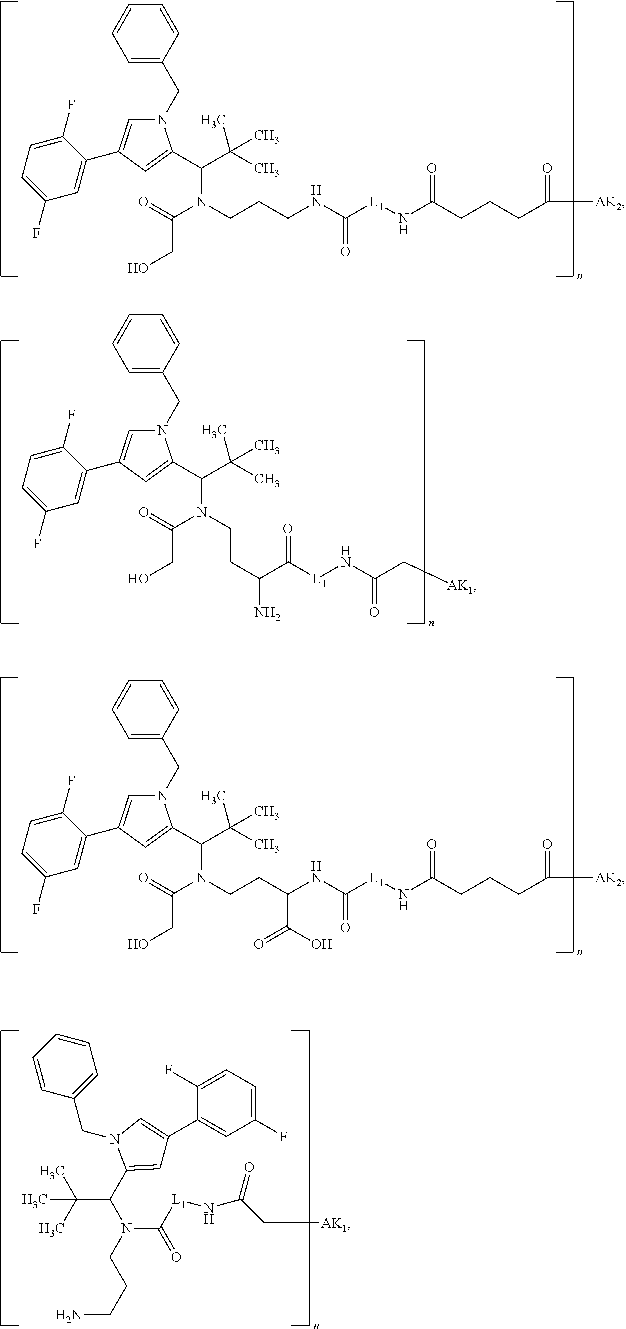

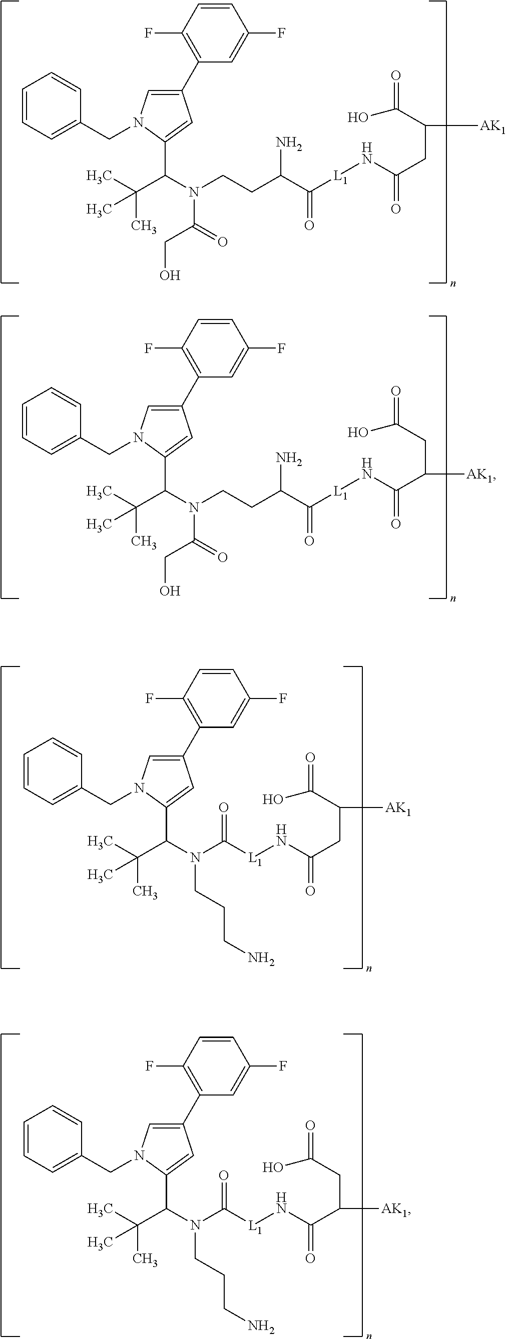

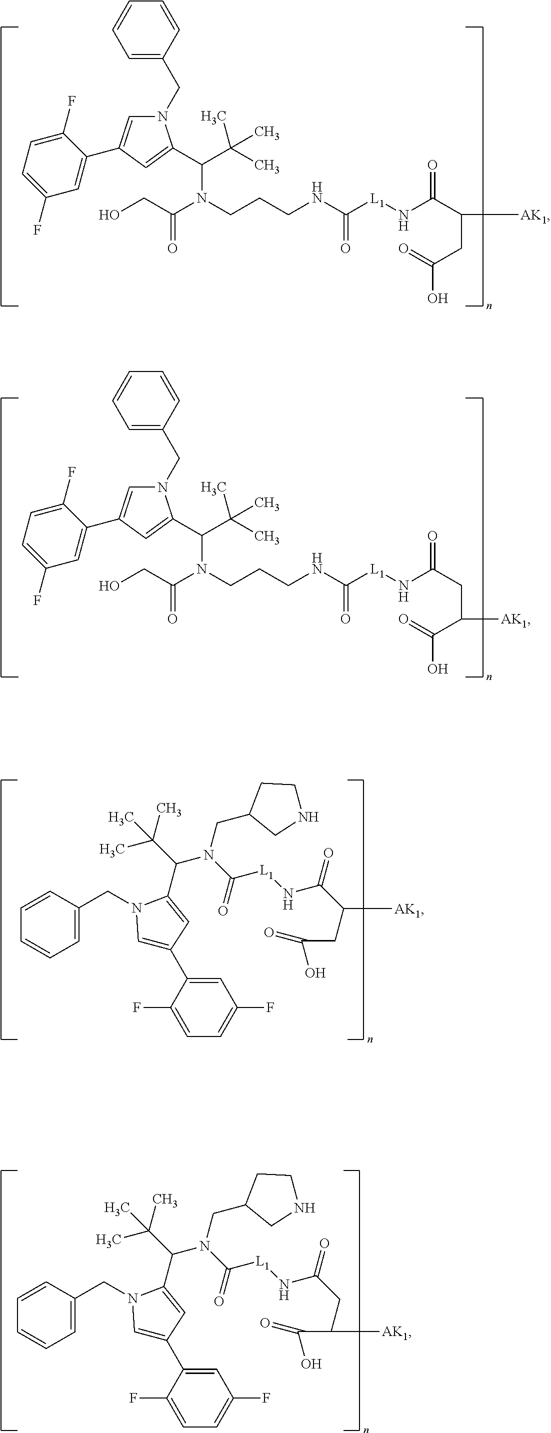

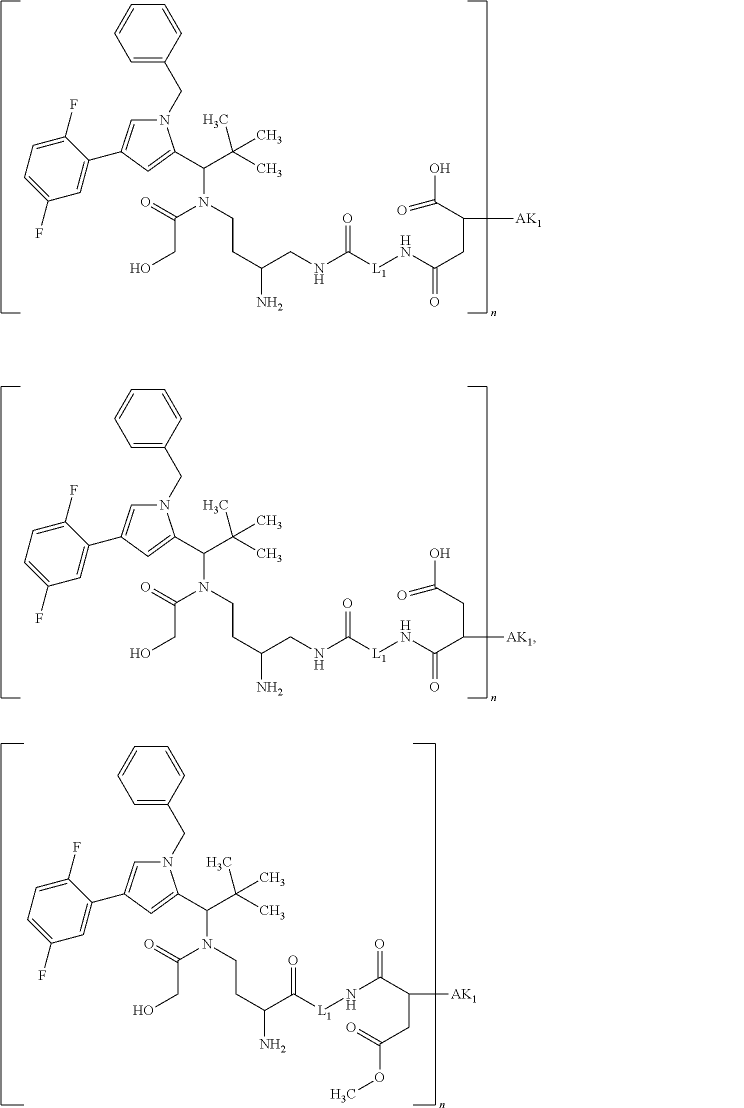

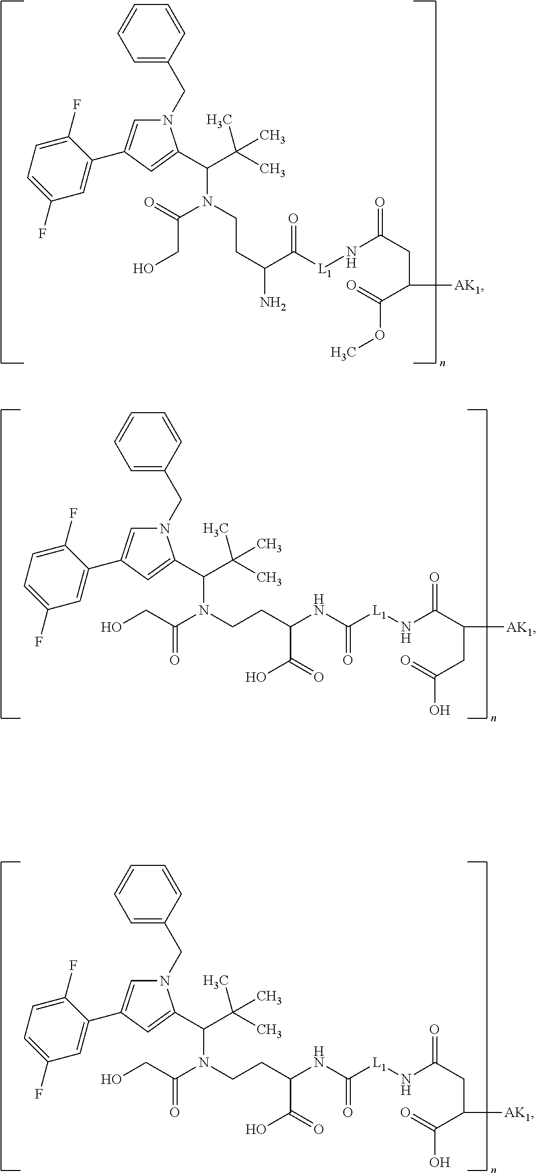

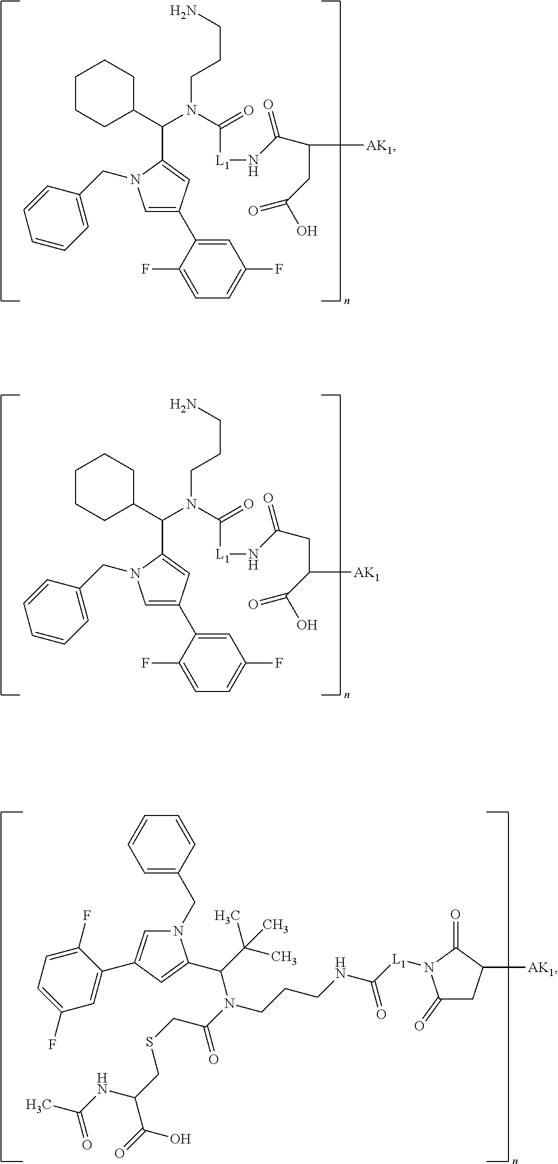

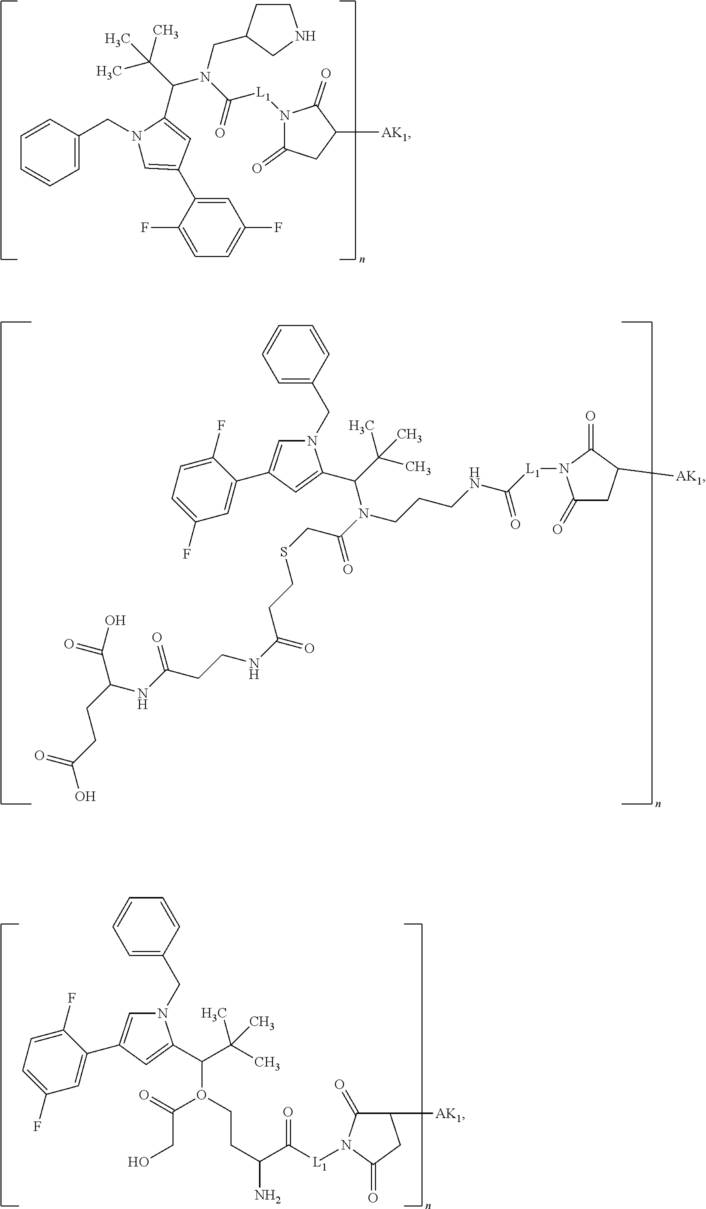

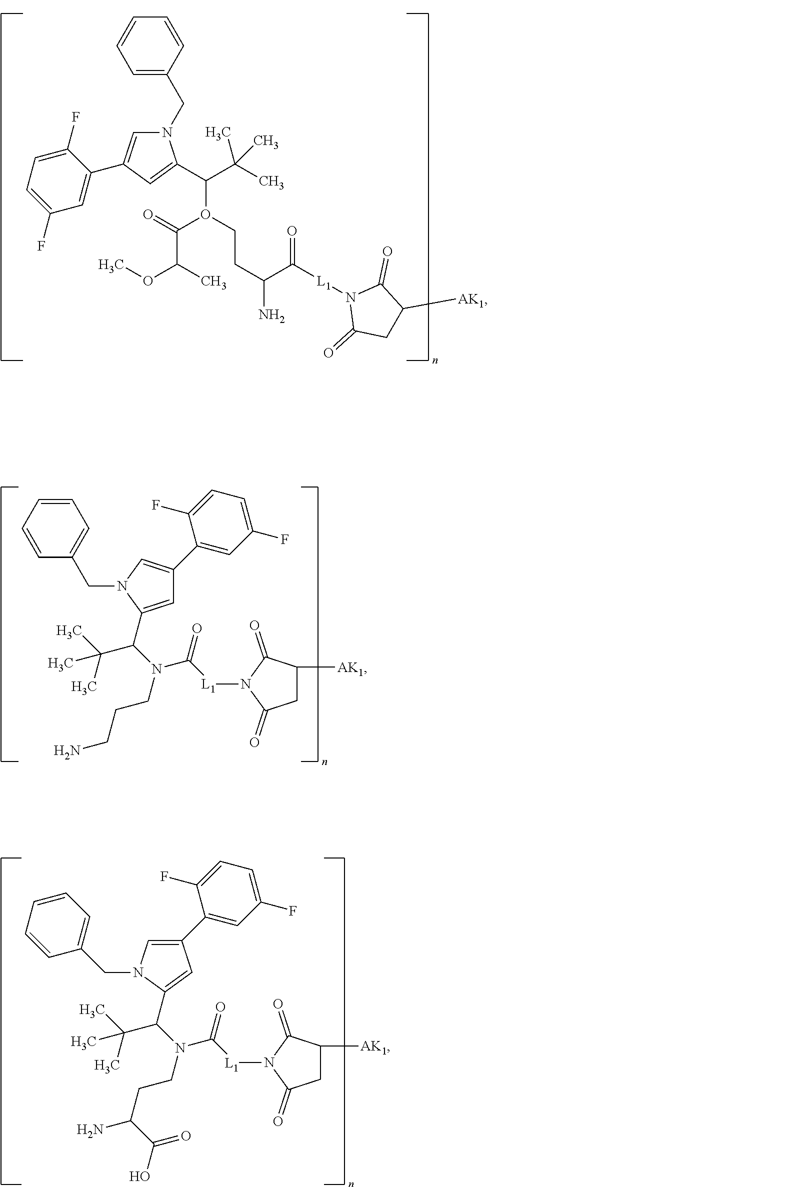

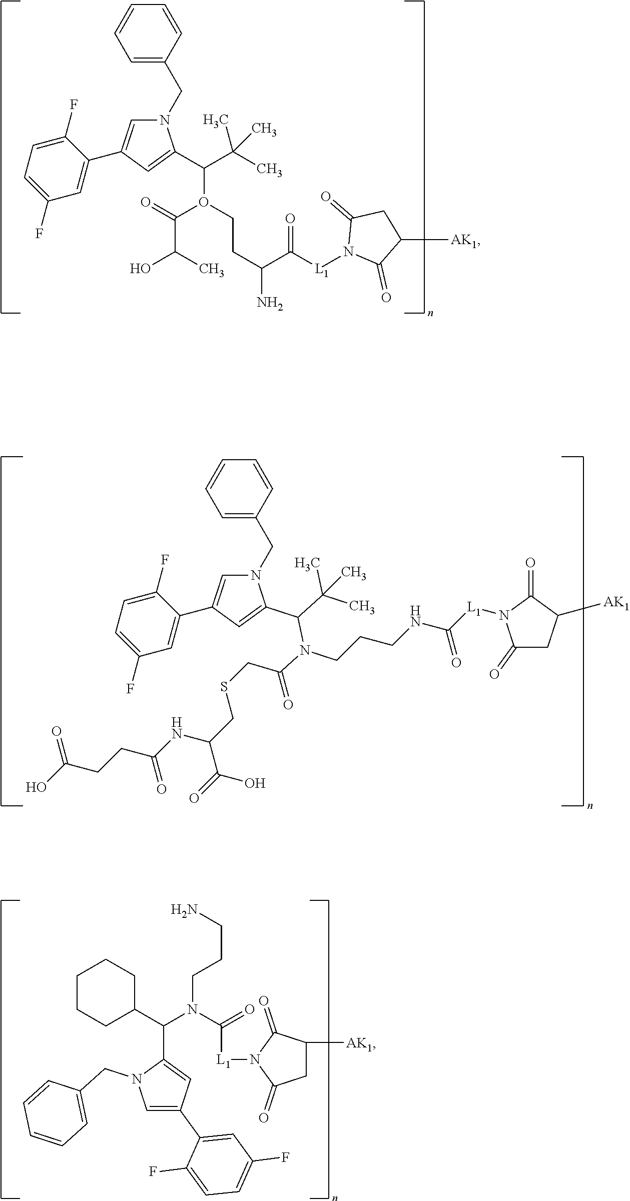

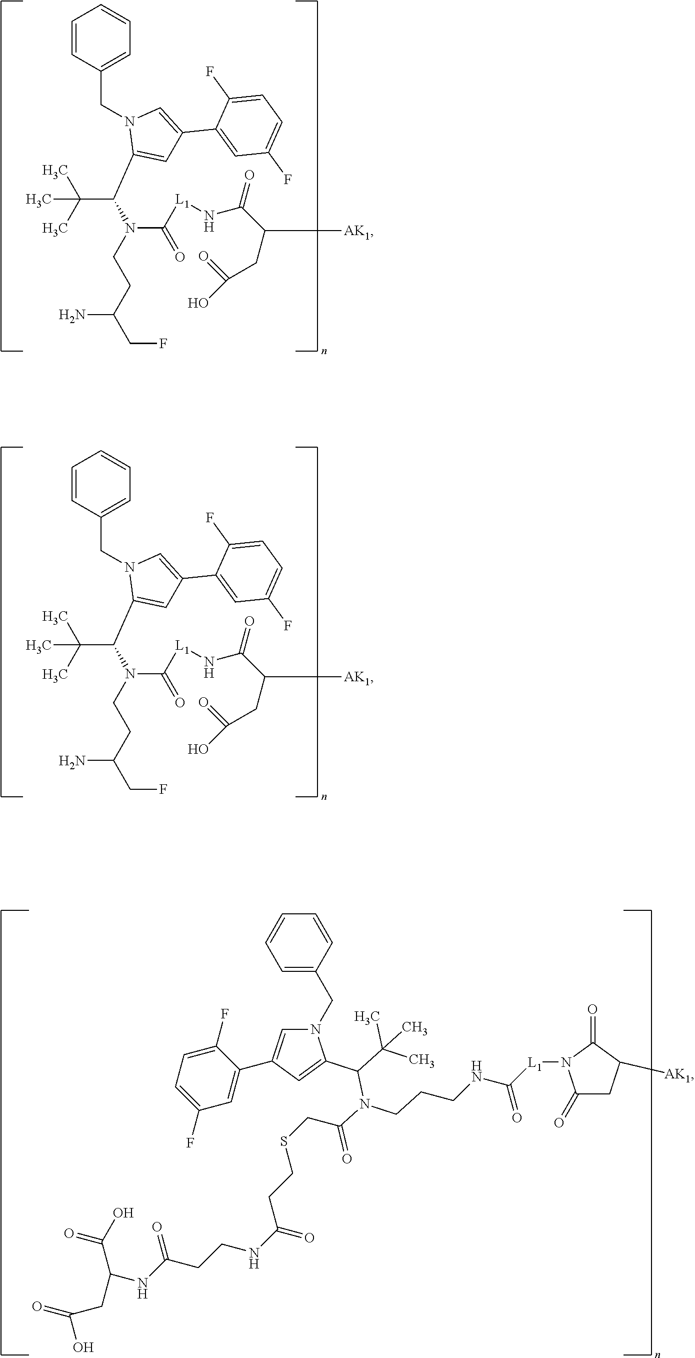

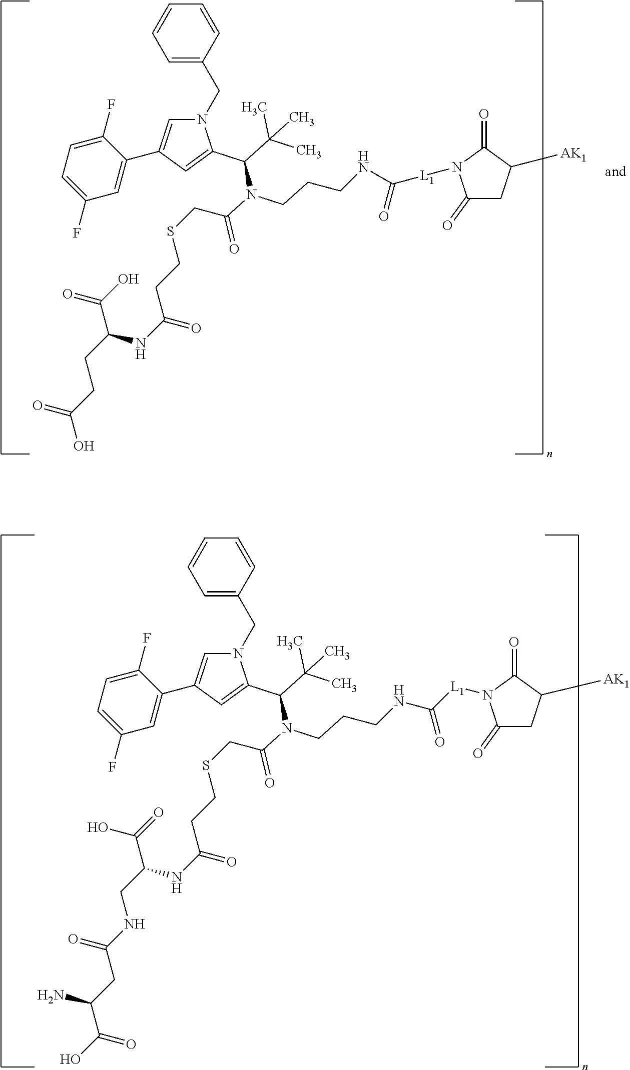

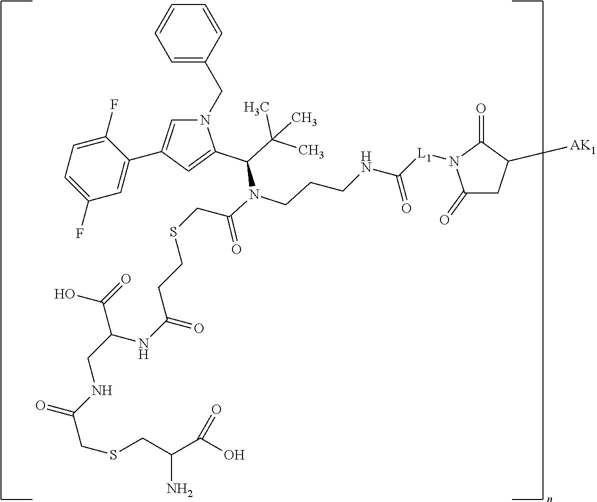

where
AK1 represents a moderately agonistically or non-agonistically acting anti-TWEAKR antibody attached via cystein and AK2 represents a moderately agonistically or non-agonistically acting anti-TWEAKR antibody attached via lysine,
n represents a number from 1 to 20 and
L₁ represents a straight-chain or branched hydrocarbon chain having 1 to 30 carbon atoms which may be interrupted once or more than once identically or differently by —O—, —S—, —C(═O)—, —S(═O)₂—, —NH—, cyclopentyl, piperidinyl, phenyl,
   where the straight-chain or branched hydrocarbon chain may be substituted by —COOH or —NH₂,
and its salts, solvates, salts of the solvates and epimers.

Here, the linker L₁ preferably represents the group
§—NH—(CH₂)₂-§§;
§—NH—(CH₂)₆-§§;
§—NH—(CH₂)₂—O—(CH₂)₂-§§;
§—NH—CH(COOH)—(CH₂)₄-§§
§—NH—NH—C(═O)—(CH₂)₅-§§;
§—NH—(CH₂)₂—C(═O)—O—(CH₂)₂-§§;
§—NH—(CH₂)₂—C(═O)—NH—(CH₂)₂-§§;
§—NH—(CH₂)₂—NH—C(═O)—CH₂-§§;
§—NH—(CH₂)₃—NH—C(═O)—CH₂-§§;
§—NH—(CH₂)₂—NH—C(═O)—(CH₂)₂-§§;
§—NH—(CH₂)₂—NH—C(═O)—(CH₂)₅-§§;
§—NH—(CH₂)₂—NH—C(═O)—CH(CH₃)—§§;
§—NH—(CH₂)₂—O—(CH₂)₂—NH—C(═O)—CH₂-§§;
§—NH—CH(COOH)—CH₂—NH—C(═O)—CH₂-§§;
§—NH—CH(COOH)—(CH₂)₂—NH—C(═O)—CH₂-§§;
§—NH—CH(COOH)—(CH₂)₄—NH—C(═O)—CH₂-§§;
§—NH—CH(COOH)—CH₂—NH—C(═O)—(CH₂)₂-§§;
§—NH—(CH₂)₂—NH—C(═O)—CH(C₂H₄COOH)—§§;
§—NH—(CH₂)₂—NH—C(═O)—((CH₂)₂—O)₃—(CH₂)₂-§§;
§—NH—(CH₂)₂—S(═O)₂—(CH₂)₂—NH—C(═O)—CH₂-§§;
§—NH—(CH₂)₂—NH—C(═O)—CH₂—NH—C(═O)—CH₂-§§;
§—NH—(CH₂)₃—NH—C(═O)—CH₂—NH—C(═O)—CH₂-§§;
§—NH—CH(COOH)—CH₂—NH—C(═O)—CH(CH₂COOH)—§§;
§—NH—(CH₂)₂—NH—C(═O)—CH(C₂H₄COOH)—NH—C(═O)—CH₂-§§;
§—NH—CH(COOH)—CH₂—NH—C(═O)—(CH₂)₂—NH—C(═O)—CH₂-§§;
§—NH—(CH₂)₂—NH—C(═O)—(CH₂)₂—CH(COOH)—NH—C(═O)—CH₂-§§;
§—NH—CH(COOH)—CH₂—NH—C(═O)—CH(CH₂OH)—NH—C(═O)—CH₂-§§;
§—NH—CH[C(═O)—NH—(CH₂)₂—O)₄—(CH₂)₂COOH]—CH₂—NH—C(═O)—CH₂-§§;
§—NH—CH(COOH)—CH₂—NH—C(═O)—((CH₂)₂—O)₄—(CH₂)₂—NH—C(═O)—CH₂-§§;
§—NH—(CH₂)₄—CH(COOH)—NH—C(═O)—CH(CH₃)—NH—C(═O)—CH(isoC₃H₇)—§§;
§—NH—(CH₂)₄—CH(COOH)—NH—C(═O)—CH(CH₃)—NH—C(═O)—CH(isoC₃H₇)—NH—C(═O)—(CH₂)₅-§§;
§—NH—(CH₂)₂—C(═O)—NH—(CH₂)₄—CH(COOH)—NH—C(═O)—CH(CH₃)—NH—C(═O)—CH(isoC₃H₇)—NH—C(═O)—CH₂-§§;
§—NH—(CH₂)₂—C(═O)—NH—(CH₂)₄—CH(COOH)—NH—C(═O)—CH(CH₃)—NH—C(═O)—CH(isoC₃H₇)—NH—C(═O)—(CH₂)₅-§§;
§—NH—(CH₂)₄—CH(COOH)—NH—C(═O)— CH[(CH₂)₃—NH—C(═O)—NH₂]—NH—C(═O)—CH(isoC₃H₇)—NH—C(═O)—(CH₂)₅-§§;
§—NH—(CH₂)₂—NH—C(═O)—(CH₂)₂—CH(COOH)—NH—C(═O)— CH(CH₃)—NH—C(═O)—CH(isoC₃H₇)—NH—C(═O)—(CH₂)₅-§§;
§—NH—CH(CH₃)—C(═O)—NH—(CH₂)₄—CH(COOH)—NH—C(═O)—CH(CH₃)—NH—C(═O)—CH(isoC₃H₇)—NH—C(═O)—(CH₂)₅-§§;
§—NH—(CH₂)₂—C(═O)—NH—(CH₂)₄—CH(COOH)—NH—C(═O)—CH[(CH₂)₃—NH—C(═O)—NH₂]—NH—C(═O)—CH(isoC₃H₇)—NH—C(═O)—(CH₂)₅-§§;
§—NH

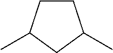

C(═O)—NH—(CH₂)₂-§§;
§—NH

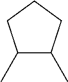

C(═O)—NH—(CH₂)₂—NH—C(═O)—CH₂-§§;
§—NH

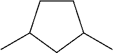

C(═O)—NH—(CH₂)₄—CH(COOH)—NH—C(═O)—CH[(CH₂)₃—NH—C(═O)—NH₂]—NH—C(═O)—CH(isoC₃H₇)—NH—C(═O)—(CH₂)₅-§§;

### §—NH

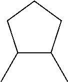 C(═O)—NH—(CH₂)₄—CH(COOH)—NH—C(═O)—CH[(CH₂)₃—NH—C(═O)—NH₂]—NH—C(═O)—CH(isoC₃Hᵥ)—NH—C(═O)—(CH₂)₅-§§;
§—NH

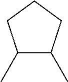

C(═O)—NH—(CH₂)₄—CH(COOH)—NH—C(═O)—CH(CH₃)—NH—C(═O)—CH(isoC₃H₇)—NH—C(═O)—(CH₂)₅-§§;
§—NH—(CH₂)₂—C(═O)—NH—CH(isoC₃H₇)—C(═O)—NH—CH[(CH₂)₃—NH—C(═O)—NH₂]—C(═O)—O

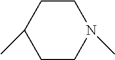

(═O)—CH₂-§§;
§—NH—(CH₂)₂—C(═O)—NH—CH(isoC₃H₇)—C(═O)—NH—CH(CH₃)—C(═O)—O

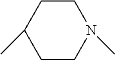

C(═O)—CH₂-§§;
§—NH—(CH₂)₂—NH—C(═O)

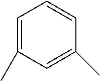

§§;
§—NH—CH(COOH)—CH₂—NH—C(═O

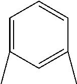

§§;
§—NH—(CH₂)₂—C(═O)—NH—CH(CH₃)—C(═O)—NH—CH[(CH₂)₃—NH—C(═O)—NH₂]—C(═O)—NH

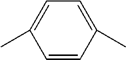

§§;
§—(CH₂)₂—C(═O)—NH—(CH₂)₂-§§;
§—(CH₂)₂—C(═O)—NH—(CH₂)₂—NH—C(═O)—CH₂-§§;
§—CH(CH₃)—NH—C(═O)—CH(isoC₃H₇)—§§;
§—CH(CH₃)—NH—C(═O)—CH(isoC₃H₇)—NH—C(═O)—CH₂-§§;
§—CH(CH₃)—NH—C(═O)—CH(isoC₃H₇)—NH—C(═O)—(CH₂)₅-§§;
§—(CH₂)₂—C(═O)—NH—((CH₂)₂—O)₄—(CH₂)₂—NH—C(═O)—CH₂-§§;
§—CH(CH₃)—NH—C(═O)—CH(isoC₃H₇)—NH—C(═O)—((CH₂)₂—O)₄—(CH₂)₂—NH—C(═O)—(CH₂)₂-§§;
§

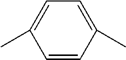

NH—C(═O)—CH(CH₃)—NH—C(═O)—CH(isoC₃H₇)—NH—C(═O)—((CH₂)₂—O)₄—(CH₂)₂—NH—C(═O)—(CH₂)₂-§§;
§—CH₂—S—(CH₂)₂—C(═O)—NH—(CH₂)₂-§§;
§—CH₂—S—(CH₂)₅—C(═O)—NH—(CH₂)₂-§§;
§—CH₂—S—CH₂CH(COOH)—NH—C(═O)—CH₂-§§;
§—CH₂—S—CH₂CH(COOH)—NH—C(═O)—(CH₂)₅-§§;
§—CH₂—S—(CH₂)₂—C(═O)—NH—((CH₂)₂—O)₂—(CH₂)₂-§§;
§—CH₂—S—(CH₂)₂—C(═O)—NH—((CH₂)₂—O)₂—(CH₂)₅-§§;
§—CH₂—S—(CH₂)₂—C(═O)—NH—(CH₂)₂—NH—C(═O)—CH₂-§§;
§—CH₂—S—(CH₂)₂—C(═O)—NH—(CH₂)₂—NH—C(═O)—CH₅-§§;
§—CH₂—S—(CH₂)₂—C(═O)—NH—CH(COOH)—CH₂—NH—C(═O)—CH₂-§§;
§—CH₂—S—CH₂CH(NH₂)—C(═O)—NH—(CH₂)₂—NH—C(═O)—(CH₂)₅-§§;
§—CH₂—S—(CH₂)₂—C(═O)—NH—((CH₂)₂—O)₂—(CH₂)₂—NH—C(═O)—CH₂-§§;
§—CH₂—S—(CH₂)₂—C(═O)—NH—((CH₂)₂—O)₄—(CH₂)₂—NH—C(═O)—CH₂-§§;
§—CH₂—S—(CH₂)₂—C(═O)—NH—((CH₂)₂—O)₂—(CH₂)₂—NH—C(═O)—(CH₂)₅-§§;
§—CH₂—S—(CH₂)₂—C(═O)—NH—((CH₂)₂—O)₄—(CH₂)₂—NH—C(═O)—(CH₂)₅-§§;
§—CH₂—S—CH₂CH(COOH)—NH—C(═O)—((CH₂)₂—O)₂—(CH₂)₂—NH—C(═O)—CH₂-§§;
§—CH₂—S—CH₂CH(COOH)—NH—C(═O)—((CH₂)₂—O)₄—(CH₂)₂—NH—C(═O)—CH₂-§§;
§—CH₂—S—CH₂CH(COOH)—NH—C(═O)—((CH₂)₂—O)₄—(CH₂)₂—NH—C(═O)—(CH₂)₂-§§;
§—CH₂—S—(CH₂)₂—CH(COOH)—NH—C(═O)—((CH₂)₂—O)₄—(CH₂)₂—NH—C(═O)—(CH₂)₂-§§;
§—CH₂—S—(CH₂)₂—C(═O)—NH—CH(C₂H₄COOH)—C(═O)—NH—(CH₂)₂—NH—C(═O)—CH₂-§§;
§—CH₂—S—CH₂CH[NH—C(═O)—(CH₂)₂—COOH]—C(═O)—NH—(CH₂)₂—NH—C(═O)—CH₂-§§;
§—CH₂—S—CH₂CH[NH—C(═O)—((CH₂)₂—O)₄—CH₃]—C(═O)—NH—(CH₂)₂—NH—C(═O)—CH₂-§§; §—CH₂—S—CH₂CH(COOH)—NH—C(═O)—CH(CH₃)—NH—C(═O)—CH(isoC₃H₇)—NH—C(═O)—CH₂-§§;
§—CH₂—S—CH₂CH[NH—C(═O)—(CH₂)₂—COOH]—C(═O)—NH—(CH₂)₂—S(═O)₂—(CH2)₂—NH—C(═O)—CH₂-§§;
§—CH₂—S—CH₂CH[NH—C(═O)—(CH₂)₂—COOH]—C(═O)—NH—((CH₂)₂—O)₄—(CH₂)₂—NH—C(═O)—CH₂-§§;
§—CH₂—S—CH₂CH[C(═O)—NH—(CH₂)₂—COOH]—NH—C(═O)—((CH₂)₂—O)₄—(CH₂)₂—NH—C(═O)—CH₂-§§;
§—CH₂—S—CH₂CH[C(═O)—NH—(CH₂)₂—COOH]—NH—C(═O)—((CH₂)₂—O)₄—(CH₂)₂—NH—C(═O)—(CH₂)₂-§§;
§—CH₂—S—CH₂CH(COOH)—NH—C(═O)—(CH₂)₂CH(COOH)—NH—C(═O)—((CH₂)₂—O)₄—(CH₂)₂—NH—C(═O)—CH₂-§§
§—CH₂—S—CH₂CH[C(═O)—NH—((CH₂)₂—O)₄—(CH₂)₂—COOH]—NH—C(═O)—((CH₂)₂—O)₄—(CH₂)₂—NH—C(═O)—CH₂-§§;
or
§—CH₂—S—CH₂CH(COOH)—NH—C(═O)—CH[(CH₂)₂—COOH]—NH—C(═O)—((CH₂)₂—O)₄—(CH₂)₂—NH—C(═O)—(CH₂)₂-§§,
where
§ represents the bond to the active compound module and
§§ represents the bond to the antibody and
isoC₃H₇ represents an isopropyl radical.

These conjugates also include their salts, solvates, salts of the solvates and epimers.

### Therapeutic Use

The hyper-proliferative diseases, for the treatment of which the compounds according to the invention may be employed, include in particular the group of cancer and tumour diseases. In the context of the present invention, these are understood to mean especially the following diseases, but without any limitation thereto: mammary carcinomas and mammary tumours (mammary carcinomas including ductal and lobular forms, also in situ), tumours of the respiratory tract (small-cell and non-small-cell pulmonary carcinoma, bronchial carcinoma), cerebral tumours (e.g. of the brain stem and of the hypothalamus, astrocytoma, ependymoma, glioblastoma, glioma, medulloblastoma, meningioma and neuro-ectodermal and pineal tumours), tumours of the digestive organs (carcinomas of the oesophagus, stomach, gall bladder, small intestine, large intestine, rectum and anal carcinomas), liver tumours (inter alia hepatocellular carcinoma, cholangiocarcinoma and mixed hepatocellular cholangiocarcinoma), tumours of the head and neck region (larynx, hypopharynx, nasopharynx, oropharynx, lips and oral cavity carcinomas, oral melanomas), skin tumours (basaliomas, spinaliomas, squamous cell carcinomas, Kaposi's sarcoma, malignant melanoma, non-melanomatous skin cancer, Merkel cell skin cancer, mast cell tumours), tumours of the stroma and connective tissue (inter alia soft tissue sarcomas, osteosarcomas, malignant fibrous histiocytomas, chondrosarcomas, fibrosarcomas, haemangiosarcomas, leiomyosarcomas, liposarcomas, lymphosarcomas and rhabdomyosarcomas), tumours of the eyes (inter alia intraocular melanoma and retinoblastoma), tumours of the endocrine and exocrine glands (e.g. of the thyroid and parathyroid glands, pancreas and salivary gland carcinomas, adenocarcinomas), tumours of the urinary tract (tumours of the bladder, penis, kidney, renal pelvis and ureter) and tumours of the reproductive organs (carcinomas of the endometrium, cervix, ovary, vagina, vulva and uterus in women and carcinomas of the prostate and testes in men). These also include proliferative diseases of the blood, the lymph system and the spinal cord, in solid form and as circulating cells, such as leukaemias, lymphomas and myeloproliferative diseases, for example acute myeloid, acute lymphoblastic, chronic lymphocytic, chronic myelogenous and hairy cell leukaemia, and AIDS-correlated lymphomas, Hodgkin's lymphomas, non-Hodgkin's lymphomas, cutaneous T cell lymphomas, Burkitt's lymphomas and lymphomas in the central nervous system.

These well-characterized diseases in humans can also occur with a comparable etiology in other mammals and can likewise be treated there with the compounds of the present invention.

The treatment of the cancer diseases mentioned above with the compounds according to the invention comprises both a treatment of the solid tumors and a treatment of metastasizing or circulating forms thereof.

In the context of this invention, the term “treatment” or “treat” is used in the conventional sense and means attending to, caring for and nursing a patient with the aim of combating, reducing, attenuating or alleviating a disease or health abnormality, and improving the living conditions impaired by this disease, as, for example, in the event of a cancer.

The present invention thus further provides for the use of the compounds of the invention for treatment and/or prevention of disorders, especially of the aforementioned disorders.

The present invention further provides for the use of the compounds according to the invention for producing a medicament for the treatment and/or prevention of disorders, especially of the aforementioned disorders.

The present invention further provides for the use of the compounds of the invention in a method for treatment and/or prevention of disorders, especially of the aforementioned disorders.

The present invention further provides a process for treatment and/or prevention of disorders, especially of the aforementioned disorders, using an effective amount of at least one of the compounds according to the invention.

The compounds of the invention can be used alone or, if required, in combination with one or more other pharmacologically active substances, provided that this combination does not lead to undesirable and unacceptable side effects. Accordingly, the present invention further provides medicaments comprising at least one of the compounds of the invention and one or more further active ingredients, especially for treatment and/or prevention of the aforementioned disorders.

For example, the compounds of the present invention can be combined with known anti-hyper-proliferative, cytostatic or cytotoxic substances for the treatment of cancer diseases. Examples of suitable combination active compounds include:

131I-chTNT, abarelix, abiraterone, aclarubicin, afatinib, aflibercept, aldesleukin, alemtuzumab, alisertib, alitretinoin, alpharadin (radium-223 chloride), altretamine, aminoglutethimide, AMP-514, amrubicin, amsacrine, anastrozole, arglabin, arsenic trioxide, asparaginase, AT9283, axitinib, azacitidine, basiliximab, belotecan, bendamustine, bevacizumab, bexarotene, bicalutamide, bisantrene, bleomycin, BMS-936559, bosutinib, bortezomib, brentuximab vedotin, buserelin, busulfan, cabazitaxel, cabozantinib, calcium folinate, calcium levofolinate, capecitabine, carboplatin, carfilzomib (proteasome inhibitor), carmofur, carmustine, catumaxomab, celecoxib, celmoleukin, cetuximab, chlorambucil, chlormadinone, chlormethine, cisplatin, cladribine, clodronic acid, clofarabine, copanlisib, crisantaspase, crizotinib, cyclophosphamide, CYC116, cyproterone, cytarabine, dacarbazine, dactinomycin, darbepoetin alfa, dabrafenib, danusertib, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, ENMD-2076, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, 1-125 seeds, ibandronic acid, ibritumomab tiuxetan, ibrutinib, idarubicin, ifosfamide, imatinib, imiquimod, INCB24360, improsulfan, interferon alpha, interferon beta, interferon gamma, ipilimumab, irinotecan, ixabepilone, lambrolizumab, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, methyl aminolevulinate, methyltestosterone, mifamurtide, miltefosine, miriplatin, mitobronitol, mitoguazone, mitolactol, mitomycin, mitotane, mitoxantrone, MLN-8054, Mps1 inhibitors (disclosed in WO2013/087579, in particular Example 01.01, WO2014/131739, in particular Example 2), nedaplatin, nelarabine, nemorubicin, nilotinib, nilutamide, nimotuzumab, nimustine, nitracrine, nivolumab, NMS-P715, NMS-P937, ofatumumab, omeprazole, oprelvekin, oxaliplatin, p53 gene therapy, paclitaxel, palbociclib, palifermin, palladium-103 seed, pamidronic acid, panitumumab, pazopanib, pegaspargase, PEG-epoetin beta (methoxy-PEG-epoetin beta), pegfilgrastim, peginterferon alfa 2b, pemetrexed, pentazocine, pentostatin, peplomycin, perfosfamide, picibanil, pirarubicin, plerixafor, plicamycin, poliglusam, polyestradiol phosphate, polysaccharide-K, ponatinib, porfimer sodium, pralatrexate, prednimustine, procarbazine, quinagolide, R763, raloxifene, raltitrexed, ranimustine, razoxane, refametinib, regorafenib, risedronic acid, rituximab, romidepsin, romiplostim, roninciclib, ruxolitinib, sargramostim, sipuleucel-T, sizofiran, sobuzoxane, sodium glycididazole, SNS-314, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, TKM-PLK1, tioguanine, tocilizumab, topotecan, toremifene, tositumomab, tozasertib, trabectedin, trametinib, trastuzumab, trastuzumab emtansine, treosulfan, tretinoin, trilostane, triptorelin, trofosfamide, tryptophan, ubenimex, valrubicin, vandetanib, vapreotide, vemurafenib, vinblastine, vincristine, Vindesine, vinflunine, vinorelbine, volasertib, vorinostat, vorozole, XL228, yttrium-90 glass microspheres, zinostatin, zinostatin stimalamer, zoledronic acid, zorubicin.

In addition, the compounds of the present invention can be combined, for example, with binders which, by way of example, can bind to the following targets: OX-40, CD137/4-1BB, DR3, IDO1/IDO2, LAG-3, CD40.

In addition, the compounds according to the invention can also be used in combination with radiotherapy and/or surgical intervention.

Generally, the following aims can be pursued with the combination of compounds of the present invention with other cytostatically or cytotoxically active agents:

improved efficacy in slowing the growth of a tumour, in reducing its size or even in completely eliminating it, compared with treatment with an individual active compound;
the possibility of using the chemotherapeutics used in a lower dosage than in the case of monotherapy;
the possibility of a more tolerable therapy with fewer side effects compared with individual administration;
the possibility of treatment of a broader spectrum of neoplastic disorders;
the achievement of a higher rate of response to the therapy;
a longer survival time of the patient compared with present-day standard therapy.

In addition, the compounds according to the invention can also be used in combination with radiotherapy and/or surgical intervention.

The present invention further provides medicaments which comprise at least one compound of the invention, typically together with one or more inert, nontoxic, pharmaceutically suitable excipients, and for the use thereof for the aforementioned purposes.

The compounds of the invention can act systemically and/or locally. For this purpose, they can be administered in a suitable manner, for example parenterally, possibly inhalatively or as implants or stents.

The compounds of the invention can be administered in administration forms suitable for these administration routes.

Parenteral administration can bypass an absorption step (for example intravenously, intraarterially, intracardially, intraspinally or intralumbally) or include an absorption (for example intramuscularly, subcutaneously, intracutaneously, percutaneously or intraperitoneally). Administration forms suitable for parenteral administration include preparations for injection and infusion in the form of solutions, suspensions, emulsions or lyophilizates. Preference is given to parenteral administration, especially intravenous administration.

In general, it has been found to be advantageous in the case of parenteral administration to administer amounts of about 0.001 to 1 mg/kg, preferably about 0.01 to 0.5 mg/kg, of body weight to achieve effective results.

It may nevertheless be necessary in some cases to deviate from the stated amounts, specifically as a function of body weight, route of administration, individual response to the active ingredient, nature of the preparation and time or interval over which administration takes place. Thus, in some cases less than the abovementioned minimum amount may be sufficient, while in other cases the upper limit mentioned must be exceeded. In the case of administration of greater amounts, it may be advisable to divide them into several individual doses over the day.

### EXAMPLES

The examples which follow illustrate the invention. The invention is not restricted to the examples.

Unless stated otherwise, the percentages in the tests and examples which follow are percentages by weight; parts are parts by weight. Solvent ratios, dilution ratios and concentration data for the liquid/liquid solutions are based in each case on volume.

If, in the description of experiments, the temperature at which the reaction is carried out is not stated, room temperature can be assumed.

### Synthesis Routes:

Exemplary for the working examples, the schemes below show exemplary synthesis routes leading to the working examples:

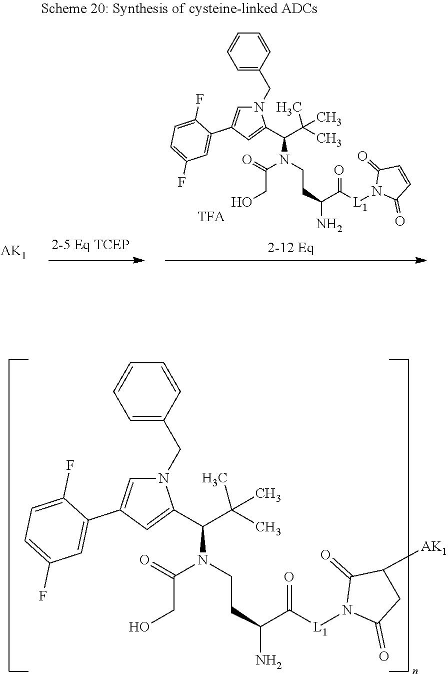

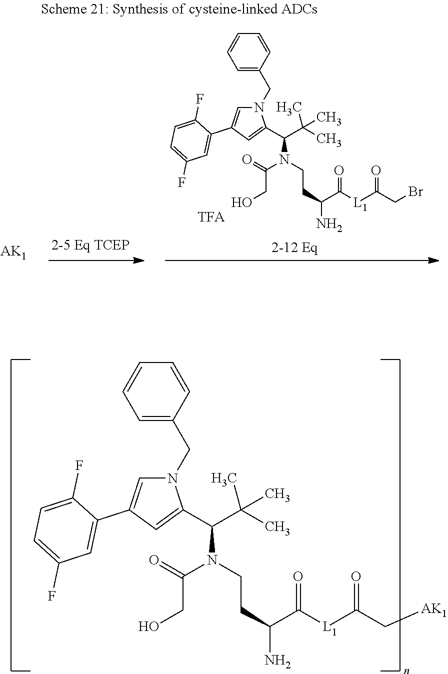

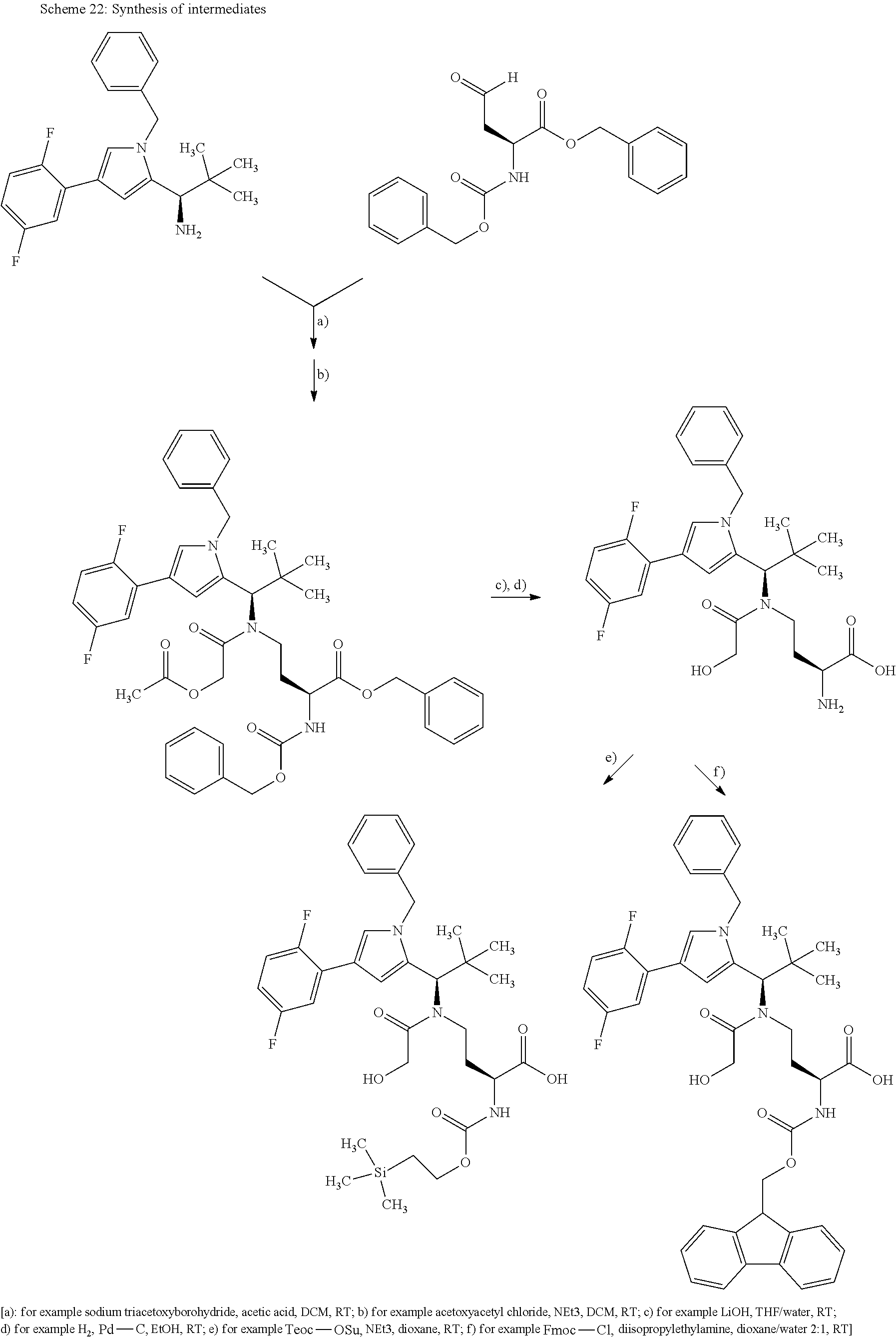

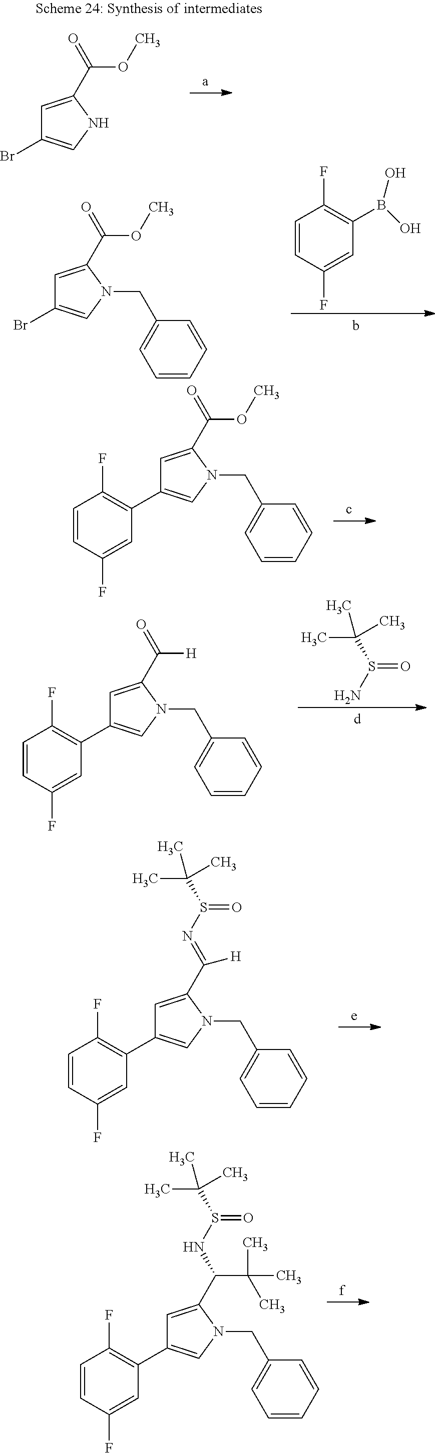

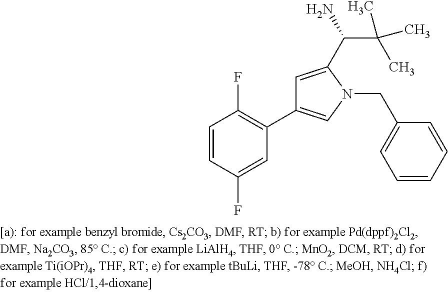 [SMILES: CC(C)(C)[C@@H](N)c1cc(-c2cc(F)ccc2F)cn1Cc1ccccc1]

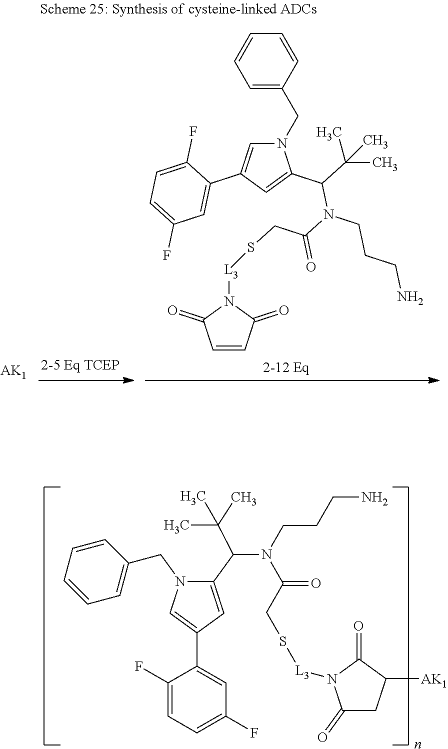

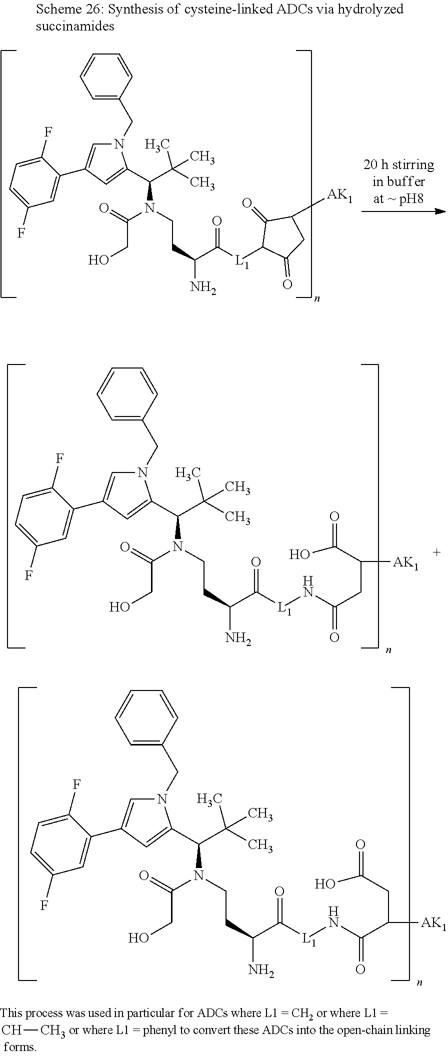

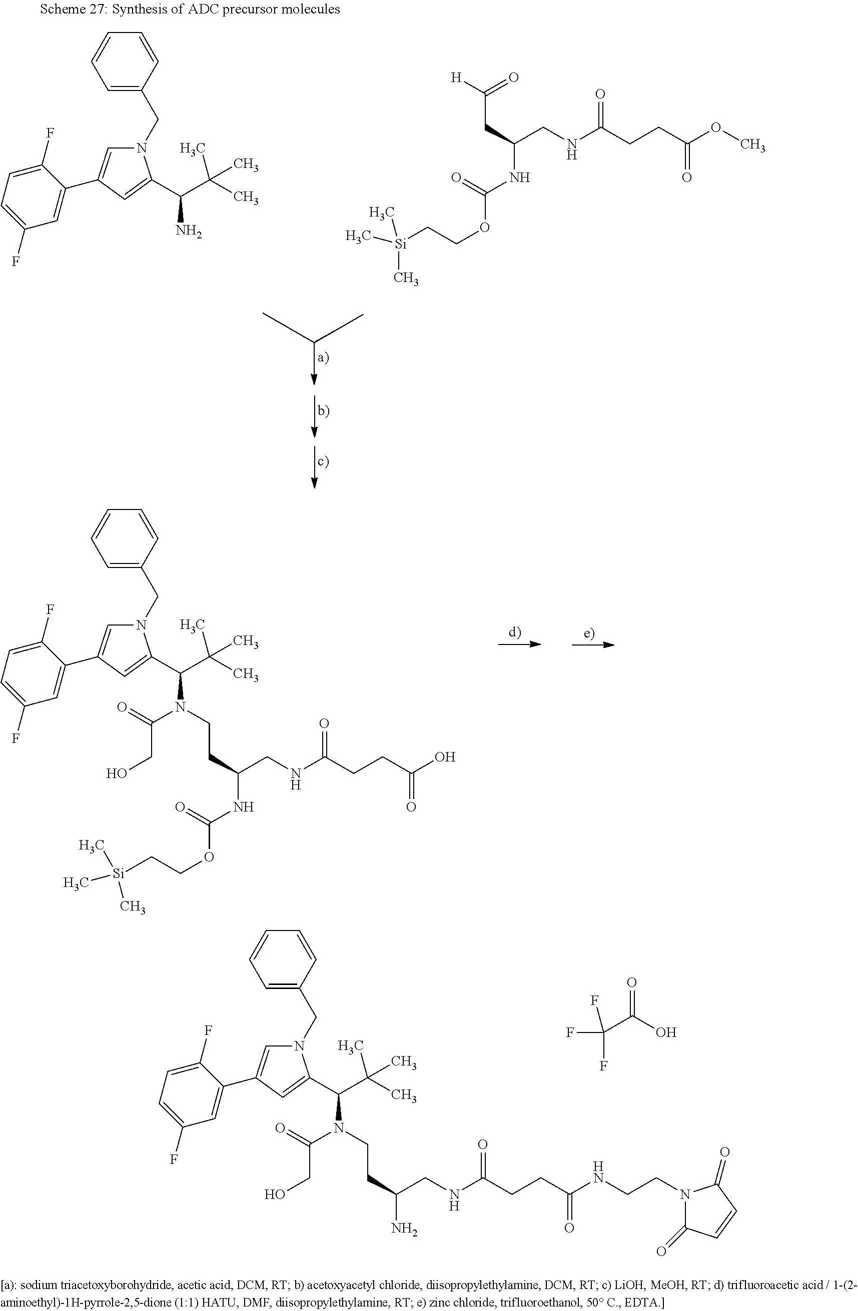

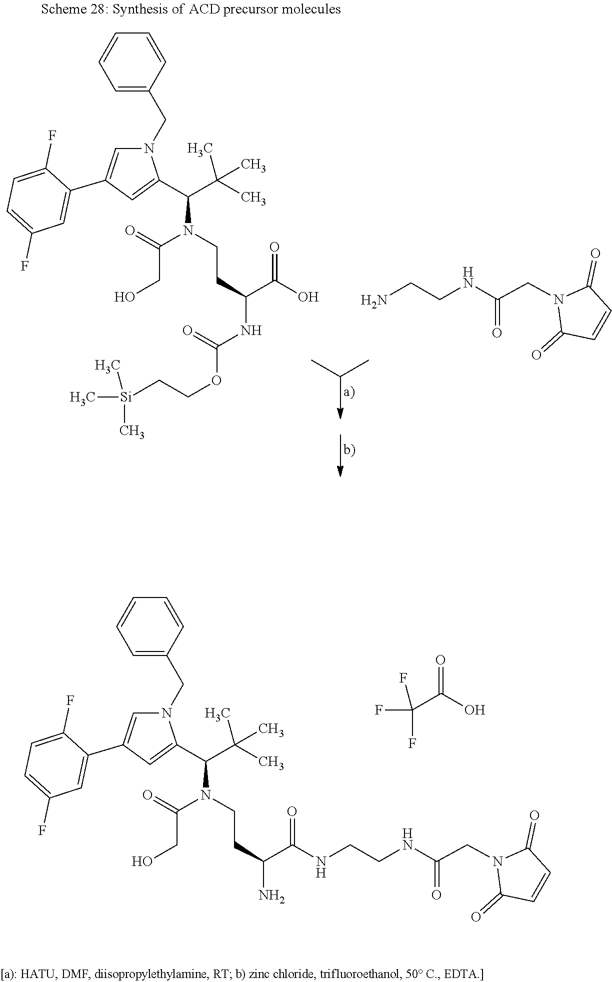

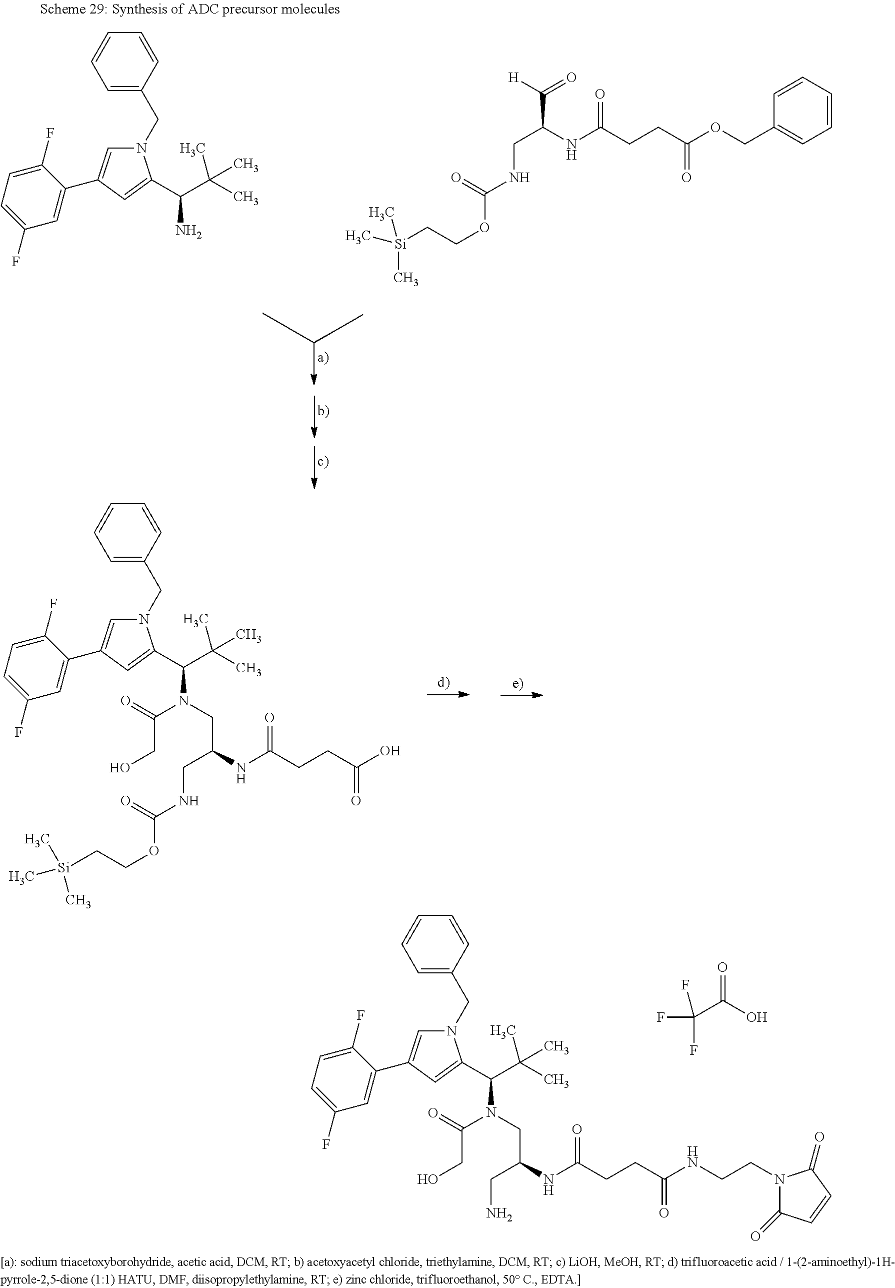 [SMILES: CC(C)(C)[C@@H](N)c1cc(-c2cc(F)ccc2F)cn1Cc1ccccc1 ; C[Si](C)(C)CCOC(=O)NC[C@@H](C=O)NC(=O)CCC(=O)OCc1ccccc1 ; CC(C)(C)[C@H](c1cc(-c2cc(F)ccc2F)cn1Cc1ccccc1)N(C[C@H](CNC(=O)OCC[Si](C)(C)C)NC(=O)CCC(=O)O)C(=O)CO ; CC(C)(C)[C@H](c1cc(-c2cc(F)ccc2F)cn1Cc1ccccc1)N(C[C@H](CN)NC(=O)CCC(=O)NCCN1C(=O)C=CC1=O)C(=O)CO ; O=C(O)C(F)(F)F]

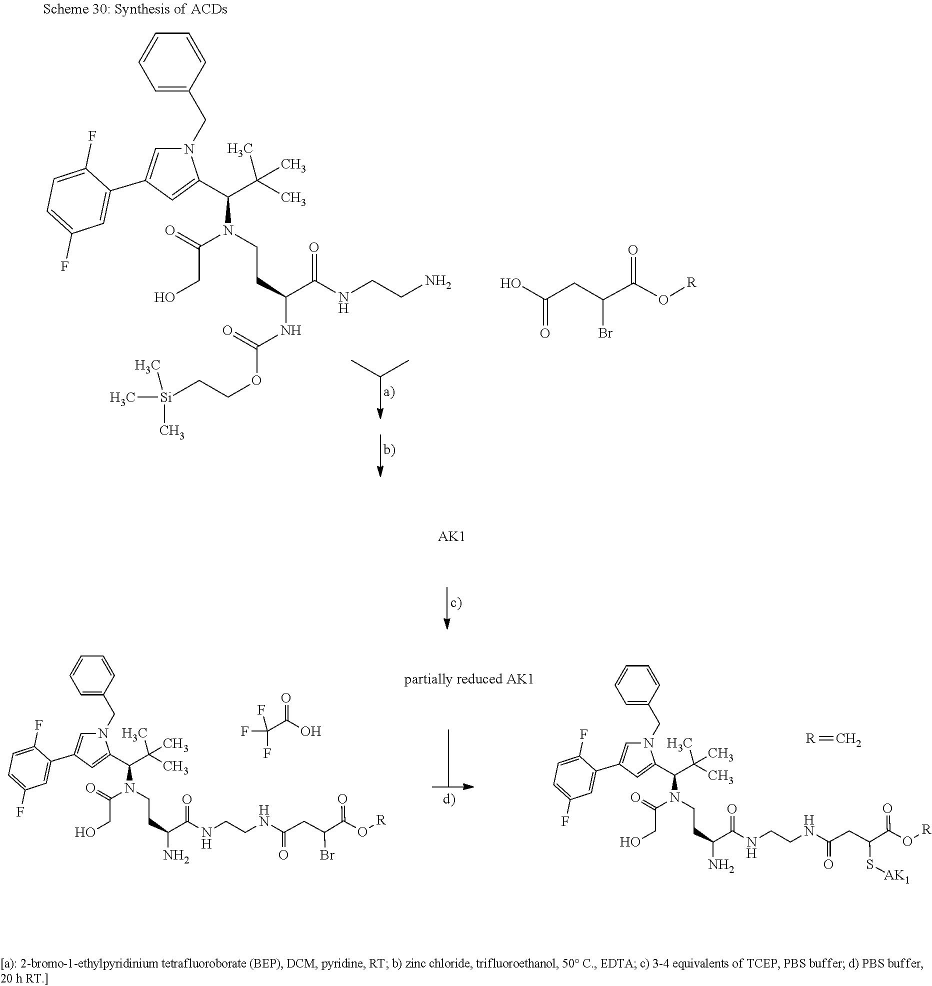

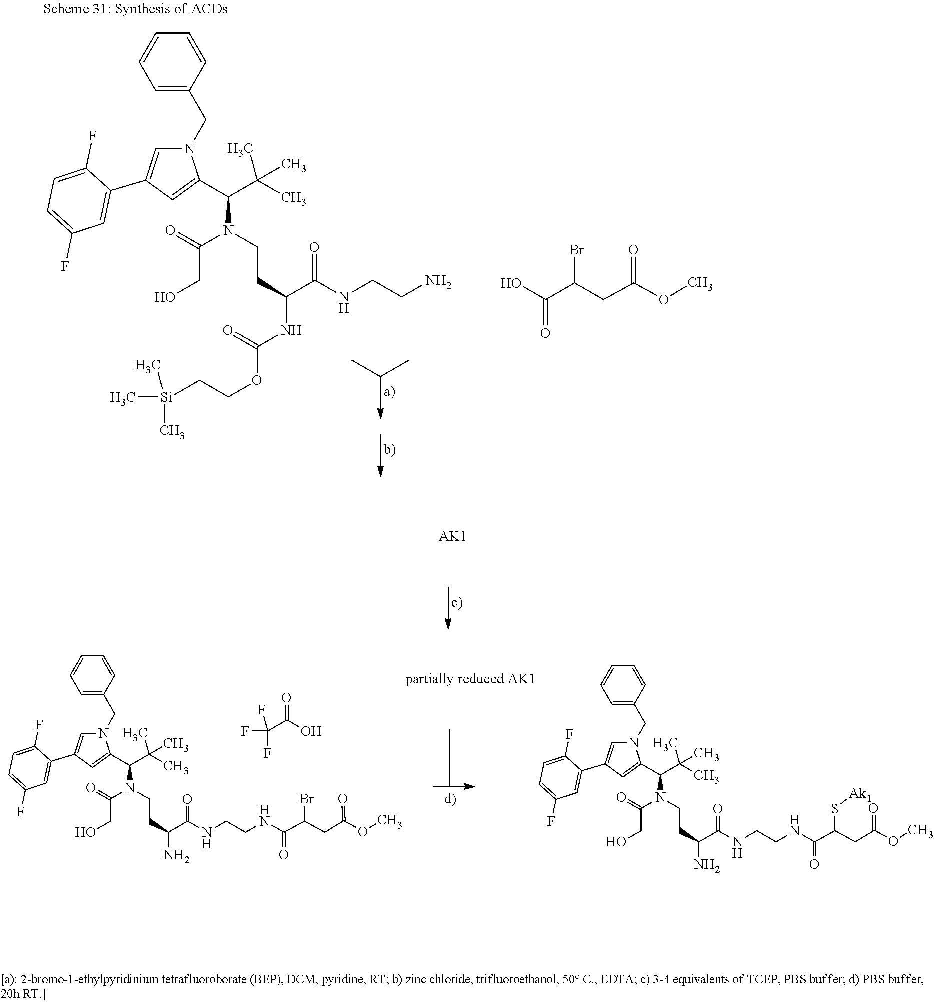

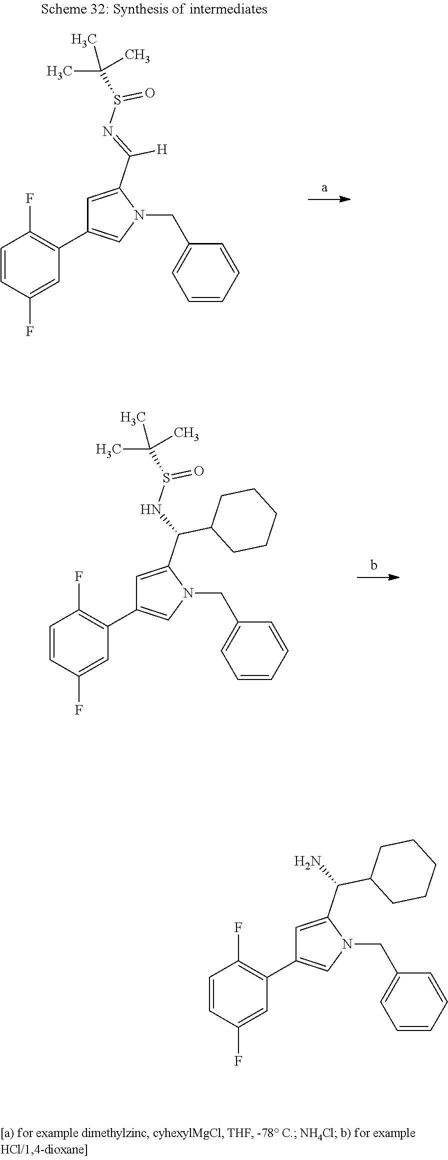 [SMILES: CC(C)(C)[S@](=O)/N=C/c1cc(-c2cc(F)ccc2F)cn1Cc1ccccc1 ; CC(C)(C)[S@](=O)N[C@@H](c1cc(-c2cc(F)ccc2F)cn1Cc1ccccc1)C1CCCCC1 ; N[C@@H](c1cc(-c2cc(F)ccc2F)cn1Cc1ccccc1)C1CCCCC1]

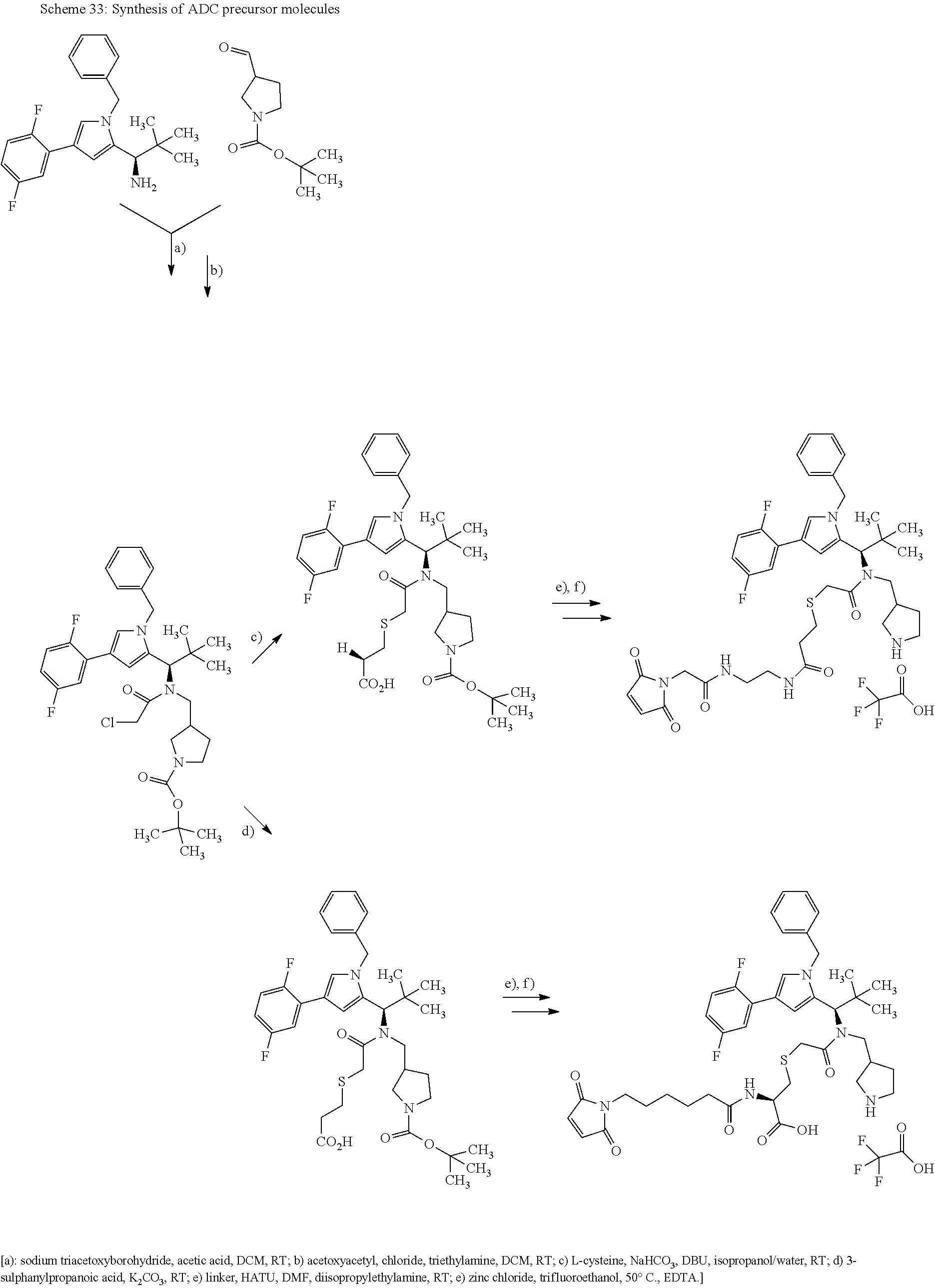

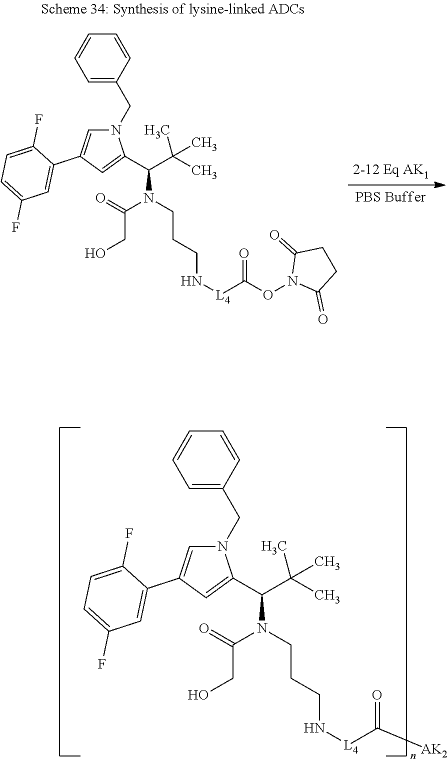

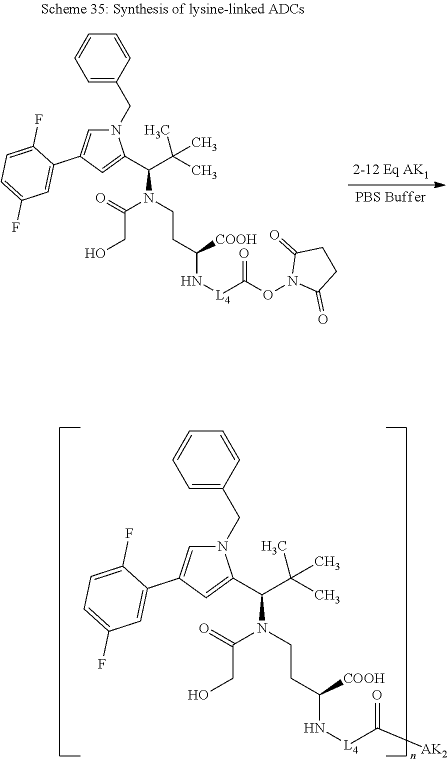

### A. EXAMPLES

### Abbreviations and Acronyms

A498 human tumour cell line
ABCB1 ATP-binding cassette sub-family B member 1 (synonym for P-gp and MDR1)
abs. absolute
Ac acetyl
ACN acetonitrile
aq. aqueous, aqueous solution
ATP adenosine triphosphate
BCRP breast cancer resistance protein, an efflux transporter
BEP 2-bromo-1-ethylpyridinium tetrafluoroborate
Boc tert-butoxycarbonyl
br. broad (in NMR)
Ex. Example
BxPC3 human tumour cell line
CI chemical ionization (in MS)
d doublet (in NMR)
d day(s)
TEC thin-layer chromatography
DCI direct chemical ionization (in MS)
DCM dichloromethane
dd doublet of doublets (in NMR)
DMAP 4-N,N-dimethylaminopyridine
DME 1,2-dimethoxy ethane
DMEM Dulbecco's Modified Eagle Medium (standardized nutrient medium for cell culture)
DMF N,N-dimethylformamide
DMSO dimethyl sulphoxide
DPBS, D-PBS, PBS Dulbecco's phosphate-buffered salt solution
   PBS=DPBS=D-PBS, pH 7.4, from Sigma, No D8537
   Composition:
   0.2 g KCl
   0.2 g KH₂PO₄ (anhyd)
   8.0 g NaCl
   1.15 g Na₂HPO₄ (anhyd)
   made up ad 11 with H₂O
dt doublet of triplets (in NMR)
DTT DL-dithiothreitol
EDC N′-(3-dimethylaminopropyl)-A-ethylcarbodiimide hydrochloride
EGFR epidermal growth factor receptor
EI electron impact ionization (in MS)
ELISA enzyme-linked immunosorbent assay
eq. equivalent(s)
ESI electrospray ionization (in MS)
ESI-MicroTofq ESI-MicroTofq (name of the mass spectrometer with Tof=time of flight and q=quadrupol)
ECS foetal calf serum
Fmoc (9H-fluoren-9-ylmethoxy)carbonyl
sat. saturated
GTP guanosine-5′-triphosphate
h hour(s)
HATU O-(7-azabenzotriazol-1-yl)-N,N,N′,N′-tetramethyluronium hexafluorophosphate
HEPES 4-(2-hydroxyethyl)piperazine-1-ethanesulphonic acid
HOAc acetic acid
HOAt 1-hydroxy-?-azabenzotriazole
HOBt 1-hydroxy-1H-benzotriazole hydrate
HOSu N-hydroxysuccinimide
HPLC high-pressure, high-performance liquid chromatography
IC₅₀ half-maximal inhibitory concentration
i.m. intramuscularly, administration into the muscle
i.v. intravenously, administration into the vein
KLP-4 human tumour cell line
cone. concentrated
KU-19-19 human tumour cell line
LC-MS liquid chromatography-coupled mass spectrometry
LLC-PK1 cells Lewis lung carcinoma pork kidney cell line
L-MDR human MDR1 transfected LLC-PK1 cells
LoVo human tumour cell line
m multiplet (in NMR)
MDR1 Multidrug resistance protein 1
MeCN acetonitrile
Me methyl
min minute(s)
MS mass spectrometry
MTT 3-(4,5-dimethylthiazol-2-yl)-2,5-diphenyl-2H-tetrazolium bromide 3
NCI-H292 human tumour cell line
NMM N-methylmorpholine
NMP N-methyl-2-pyrrolidinone
NMR nuclear magnetic resonance spectrometry
NMRI mouse strain originating from the Naval Medical Research Institute (NMRI)
Nude mice nude mice (experimental animals)
NSCLC non small cell lung cancer
PBS phosphate-buffered salt solution
Pd/C palladium on activated carbon
P-gp P-gycoprotein, a transporter protein
PNGaseF enzyme for cleaving sugar
quant. quantitative (in yield)
quart quartet (in NMR)
quint quintet (in NMR)
R_{f} retention index (in TLC)
RT room temperature
Rₜ retention time (in HPLC)
s singlet (in NMR)
s.c. subcutaneously, administration under the skin
SCID mice test mice with severe combined immunodeficiency
SK-HEP-1 human tumour cell line
t triplet (in NMR)
TBAF tetra-n-butylammonium fluoride
TEMPO (2,2,6,6-tetramethylpiperidin-1-yl)oxyl
tert tertiary
TFA trifluoroacetic acid
THF tetrahydrofuran
T3P® 2,4,6-tripropyl-1,3,5,2,4,6-trioxatriphosphinane 2,4,6-trioxide
UV ultraviolet spectrometry
v/v volume to volume ratio (of a solution)
Z Benzyloxycarbonyl
786-O human tumour cell line

### HPLC and LC-MS Methods:

### Method 1 (LC-MS):

Instrument: Waters ACQUITY SQD UPLC System; column: Waters Acquity UPLC HSS T3 1.8μ 50×1 mm; mobile phase A: 1 l of water+0.25 ml of 99% strength formic acid, mobile phase B: 11 of acetonitrile+0.25 ml of 99% strength formic acid; gradient: 0.0 min 90% A→1.2 min 5% A→2.0 min 5% A oven: 50° C.; flow rate: 0.40 ml/min; UV detection: 208-400 nm.

### Method 2 (LC-MS):

MS instrument type: Waters Synapt G2S; UPLC instrument type: Waters Acquity I-CLASS; column: Waters, BEH300, 2.1×150 mm, C18 1.7 μm; mobile phase A: 1 l of water+0.01% formic acid; mobile phase B: 1 l of acetonitrile+0.01% formic acid; gradient: 0.0 min 2% B→1.5 min 2% B→8.5 min 95% B→10.0 min 95% B; oven: 50° C.; flow rate: 0.50 ml/min; UV detection: 220 nm

### Method 3 (LC-MS):

MS instrument: Waters (Micromass) QM; HPLC instrument: Agilent 1100 Series; column: Agilent ZORBAX Extend-C18 3.0×50 mm 3.5-micron; mobile phase A: 1 l of water+0.01 mol of ammonium carbonate, mobile phase B: 1 l of acetonitrile; gradient: 0.0 min 98% A→0.2 min 98% A→3.0 min 5% A→4.5 min 5% A; oven: 40° C.; flow rate: 1.75 ml/min; UV detection: 210 nm

### Method 4 (LC-MS):

MS instrument type: Waters Synapt G2S; UPLC instrument type: Waters Acquity I-CLASS; column: Waters, HSST3, 2.1×50 mm, C18 1.8 μm; mobile phase A: 1 l of water+0.01% formic acid; mobile phase B: 1 l of acetonitrile+0.01% formic acid; gradient: 0.0 min 10% B→0.3 min 10% B→1.7 min 95% B→2.5 min 95% B; oven: 50° C.; flow rate: 1.20 ml/min; UV detection: 210 nm

### Method 5 (LC-MS):

Instrument: Waters ACQUITY SQD UPLC System; column: Waters Acquity UPLC HSS T3 1.8μ 50×1 mm; mobile phase A: 1 l of water+0.25 ml of 99% strength formic acid, mobile phase B: 11 of acetonitrile+0.25 ml of 99% strength formic acid; gradient: 0.0 min 95% A→6.0 min 5% A→7.5 min 5% A oven: 50° C.; flow rate: 0.35 ml/min; UV detection: 210-400 nm.

### Method 6 (LC-MS):

Instrument: Micromass Quattro Premier with Waters UPLC Acquity; column: Thermo Hypersil GOLD 1.9μ 50×1 mm; mobile phase A: 11 of water+0.5 ml of 50% strength formic acid, mobile phase B: 1 l of acetonitrile+0.5 ml of 50% strength formic acid; gradient: 0.0 min 97% A→0.5 min 97% A→3.2 min 5% A→4.0 min 5% A oven: 50° C.; flow rate: 0.3 ml/min; UV detection: 210 nm.

### Method 7 (LC-MS):

Instrument: Agilent MS Quad 6150; HPLC: Agilent 1290; column: Waters Acquity UPLC HSS T3 1.8μ 50×2.1 mm; mobile phase A: 1 l of water+0.25 ml of 99% strength formic acid, mobile phase B: 1 l of acetonitrile+0.25 ml of 99% strength formic acid; gradient: 0.0 min 90% A→0.3 min 90% A→1.7 min 5% A→3.0 min 5% A oven: 50° C.; flow rate: 1.20 ml/min; UV detection: 205-305 nm.

### Method 8 (LC-MS):

MS instrument type: Waters Synapt G2S; UPLC instrument type: Waters Acquity I-CLASS; column: Waters, HSST3, 2.1×50 mm, C18 1.8 μm; mobile phase A: 1 l of water+0.01% formic acid; mobile phase B: 1 l of acetonitrile+0.01% formic acid; gradient: 0.0 min 2% B→2.0 min 2% B→13.0 min 90% B→15.0 min 90% B; oven: 50° C.; flow rate: 1.20 ml/min; UV detection: 210 nm

Method 9: LC-MS-Prep purification method for Examples 181-191 (Method LIND-LC-MS-Prep)

MS instrument: Waters, HPLC instrument: Waters (column Waters X-Bridge C18, 19 mm×50 mm, 5 μm, mobile phase A: water+0.05% ammonia, mobile phase B: acetonitrile (ULC) with gradient; flow rate: 40 ml/min; UV detection: DAD; 210-400 nm).

or

MS instrument: Waters, HPLC instrument: Waters (column Phenomenex Luna 5μ C18(2) 100A, AXIA Tech. 50×21.2 mm, mobile phase A: water+0.05% formic acid, mobile phase B: acetonitrile (ULC) with gradient; flow rate: 40 ml/min; UV detection: DAD; 210-400 nm).

Method 10: LC-MS Analysis Method for Examples 181-191 (LIND_SQD_SB_AQ)

MS instrument: Waters SQD; Instrument HPLC: Waters UPLC; column: Zorbax SB-Aq (Agilent), 50 mm×2.1 mm, 1.8 μm; mobile phase A: water+0.025% formic acid, mobile phase B: acetonitrile (ULC)+0.025% formic acid; gradient: 0.0 min 98% A—0.9 min 25% A—1.0 min 5% A—1.4 min 5% A—1.41 min 98% A—1.5 min 98% A; oven: 40° C.; flow rate: 0.600 ml/min; UV detection: DAD; 210 nm.

### Method 11 (HPLC):

Instrument: HP1100 Series
column: Merck Chromolith SpeedROD RP-18e, 50-4.6 mm, Cat. No. 1.51450.0001, precolumn Chromolith Guard Cartridge Kit, RP-18e, 5-4.6 mm, Cat. No. 1.51470.0001
gradient: flow rate 5 ml/min
   injection volume 5 μl
   solvent A: HClO4 (70% strength) in water (4 ml/1)
   solvent B: acetonitrile
   start 20% B
   0.50 min 20% B
   3.00 min 90% B
   3.50 min 90% B
   3.51 min 20% B
   4.00 min 20% B
   column temperature: 40° C.
wavelength: 210 nm

### Method 12 (LC-MS):

MS instrument type: Thermo Scientific FT-MS; UHPLC+ instrument type: Thermo Scientific UltiMate 3000; column: Waters, HSST3, 2.1×75 mm, C18 1.8 μm; mobile phase A: 1 l of water+0.01% formic acid; mobile phase B: 1 l of acetonitrile+0.01% formic acid; gradient: 0.0 min 10% B→2.5 min 95% B→3.5 min 95% B; oven: 50° C.; flow rate: 0.90 ml/min; UV detection: 210 nm/optimum integration path 210-300 nm

### Method 13: (LC-MS):

MS instrument: Waters (Micromass) Quattro Micro; Instrument Waters UPLC Acquity; column: Waters BEH C18 1.7μ 50×2.1 mm; mobile phase A: 1 l of water+0.01 mol ammonium formate, mobile phase B: 1 l of acetonitrile; gradient: 0.0 min 95% A→0.1 min 95% A→2.0 min 15% A→2.5 min 15% A→2.51 min 10% A→3.0 min 10% A; oven: 40° C.; flow rate: 0.5 ml/min; UV detection: 210 nm

### Method 14: (LC-MS):

MS instrument type: ThermoFisherScientific LTQ-Orbitrap-XL; HPLC instrument type: Agilent 1200SL; column: Agilent, POROSHELL 120, 3×150 mm, SB—C18 2.7 μm; mobile phase A: 1 l of water+0.1% trifluoroacetic acid; mobile phase B: 1 l of acetonitrile+0.1% trifluoroacetic acid; gradient: 0.0 min 2% B→0.3 min 2% B→5.0 min 95% B→10.0 min 95% B; oven: 40° C.; flow rate: 0.75 ml/min; UV detection: 210 nm

All reactants or reagents whose preparation is not described explicitly hereinafter were purchased commercially from generally accessible sources. For all other reactants or reagents whose preparation likewise is not described hereinafter and which were not commercially obtainable or were obtained from sources which are not generally accessible, a reference is given to the published literature in which their preparation is described.

### Starting Materials and Intermediates

### Intermediate C2

### tert-Butyl (2S)-4-({(1R)-1-[1-benzyl-4-(2,5-difluorophenyl)-1H-imidazol-2-yl]-2,2-dimethylpropyl}amino)-2-[(tert-butoxy carbonyl)amino]butanoate

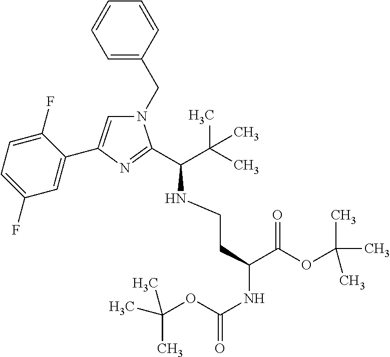 [SMILES: CC(C)(C)OC(=O)N[C@@H](CCN[C@@H](c1nc(-c2cc(F)ccc2F)cn1Cc1ccccc1)C(C)(C)C)C(=O)OC(C)(C)C]

4.22 g (14.5 mmol) of tert-butyl N-(tert-butoxycarbonyl)-L-homoserinate were dissolved in 180 ml of dichloromethane, and 3.5 ml of pyridine and 9.2 g (21.7 mmol) of 1,1,1-triacetoxy-1 lambda⁵,2-benziodoxol-3(1H)-one were then added. The mixture was stirred at RT for 1 h and then diluted with 500 ml of dichloromethane and extracted twice with 10% strength sodium thiosulphate solution and then successively twice with 5% strength citric acid and twice with 10% strength sodium bicarbonate solution. The organic phase was separated off, dried over magnesium sulphate and then concentrated under reduced pressure. The residue was taken up in DCM and a mixture of diethyl ether and n-pentane was added. The precipitate was filtered off and the filtrate was then concentrated and lyophilized from acetonitrile/water. This gave 3.7 g (93%) of tert-butyl (2S)-2-[(tert-butoxycarbonyl)amino]-4-oxobutanoate which were used without further purification for the next step. (R_{f} value: 0.5 (DCM/methanol 95/5)).

3.5 g (9.85 mmol) of Intermediate C1 were dissolved in 160 ml of DCM, and 3.13 g (14.77 mmol) of sodium triacetoxyborohydride and 0.7 ml of acetic acid were added. After 5 min of stirring at RT, 3.23 g (11.85 mmol) of tert-butyl (2S)-2-[(tert-butoxycarbonyl)amino]-4-oxobutanoate were added and the mixture was stirred at RT for a further 30 min. The solvent was then evaporated under reduced pressure and the residue was taken up in acetonitrile/water. The precipitated solid was filtered off and dried, giving 5.46 g (84%) of the title compound.

HPLC (Method 11): Rₜ=2.5 min;

LC-MS (Method 1): Rₜ=1.13 min; MS (ESIpos): m/z=613 (M+H)⁺.

### Intermediate C11

### R/S-(11-{(1R)-1-[1-Benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-homocysteine/trifluoroacetate (1:1)

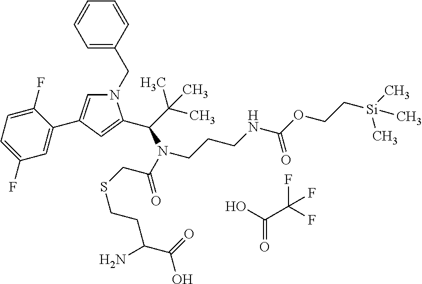

990.0 mg (2.79 mmol) of (1R)-1-[1-benzyl-4-(2,5-difluorophenyl)-1H-pyrrol-2-yl]-2,2-dimethylpropan-1-amine were initially charged in 15.0 ml of dichloromethane, and 828.8 mg (3.91 mmol) of sodium triacetoxyborohydride and 129.9 mg (3.21 mmol) of acetic acid were added, and the mixture was stirred at RT for 5 min. 698.1 mg (3.21 mmol) of 2-(trimethylsilyl)ethyl (3-oxopropyl)carbamate (Intermediate L58) dissolved in 15.0 ml of dichloromethane were added, and the reaction mixture was stirred at RT overnight. The reaction mixture was diluted with ethyl acetate and the organic phase was washed in each case twice with saturated sodium carbonate solution and saturated NaCl solution. The organic phase was dried over magnesium sulphate and the solvent was evaporated under reduced pressure. The residue was purified on silica gel (mobile phase: dichloromethane/methanol=100:2). The solvents were evaporated under reduced pressure and the residue was dried under high vacuum. This gave 1.25 g (73% of theory) of the compound 2-(trimethylsilyl)ethyl [3-({(1R)-1-[1-benzyl-4-(2,5-difluorophenyl)-1H-pyrrol-2-yl]-2,2-dimethylpropyl}aminopropyl]carbamate.

LC-MS (Method 1): Rₜ=1.09 min; MS (ESIpos): m/z=556 (M+H)⁺.

151.4 mg (1.5 mmol) of triethylamine and 161.6 mg (1.43 mmol) of chloroacetyl chloride were added to 400.0 mg (0.65 mmol) of 2-(trimethylsilyl)ethyl [3-({(1R)-1-[1-benzyl-4-(2,5-difluorophenyl)-1H-pyrrol-2-yl]-2,2-dimethylpropyl}aminopropyl]carbamate. The reaction mixture was stirred at RT overnight. Ethyl acetate was added to the reaction mixture and the organic phase was washed three times with water and once with saturated NaCl solution. The organic phase was dried over magnesium sulphate and the solvent was evaporated under reduced pressure. The residue was purified by silica gel chromatography (mobile phase: cyclohexane/ethyl acetate=3:1). The solvents were evaporated under reduced pressure and the residue was dried under high vacuum. This gave 254.4 mg (57% of theory) of the compound 2-(trimethylsilyl)ethyl {3-[{(1R)-1-[1-benzyl-4-(2,5-difluorophenyl)-1H-pyrrol-2-yl]-2,2-dimethylpropyl}(chloroacetyl)amino]propyl}carbamate.

LC-MS (Method 1): Rₜ=1.49 min; MS (ESIneg): m/z=676 (M+HCOO⁻)⁻.

117.4 mg (0.19 mmol) of 2-(trimethylsilyl)ethyl {3-[{(1R)-1-[1-benzyl-4-(2,5-difluorophenyl)-1H-pyrrol-2-yl]-2,2-dimethylpropyl}(chloroacetyl)amino]propyl}carbamate were dissolved in 10.0 ml of isopropanol, and 928.4 μl of 1M NaOH and 50.2 mg (0.37 mmol) of DL-homocysteine were added. The reaction mixture was stirred at 50° C. for 4.5 h. Ethyl acetate was added to the reaction mixture and the organic phase was washed with saturated sodium bicarbonate solution and saturated NaCl solution. The organic phase was dried over magnesium sulphate and the solvent was evaporated under reduced pressure. The residue was purified by preparative RP-HPLC (column: Reprosil 250×40; 10μ, flow rate: 50 ml/min, MeCN/water, 0.1% TFA). The solvents were evaporated under reduced pressure and the residue was dried under high vacuum. This gave 75.3 mg (48% of theory) of the title compound.

LC-MS (Method 1): Rₜ=1.24 min; MS (ESIpos): m/z=731 (M+H)⁺.

¹H-NMR (400 MHz, DMSO-d₆): δ [ppm]=0.03 (s, 9H), 0.40 (m, 1H), 0.75-0.91 (m, 11H), 1.30 (m, 1H), 1.99-2.23 (m, 2H), 2.63-2.88 (m, 4H), 3.18-3.61 (m, 5H), 3.79-4.10 (m, 3H), 4.89 (d, 1H), 4.89 (d, 1H), 5.16 (d, 1H), 5.56 (s, 1H), 6.82 (m, 1H), 6.91 (s, 1H), 6.97 (m, 1H), 7.13-7.38 (m, 6H), 7.49 (s, 1H), 7.63 (m, 1H), 8.26 (s, 3H).

### Intermediate C12

### R/S-[(8S)-11-{(1R)-1-[1-Benzyl-4-(2,5-difluorophenyl)-1H-pyrrol-2-yl]-2,2-dimethylpropyl}-8-carboxy-2,2-dimethyl-6,12-dioxo-5-oxa-7,11-diaza-2-silatridecan-13-yl]homocysteine

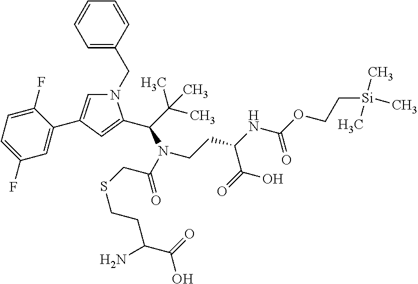 [SMILES: CC(C)(C)[C@H](c1cc(-c2cc(F)ccc2F)cn1Cc1ccccc1)N(CC[C@H](NC(=O)OCC[Si](C)(C)C)C(=O)O)C(=O)CSCCC(N)C(=O)O]

The synthesis was carried out analogously to the synthesis of Intermediate C11 using methyl (2S)-4-oxo-2-({[2-(trimethylsilyl)ethoxy]carbonyl}amino)butanoate (Intermediate L57) and Intermediate C52 as starting materials.

LC-MS (Method 1): Rₜ=1.18 min; MS (ESIpos): m/z=775 (M+H)⁺.

### Intermediate C52

### (1R)-1-[1-Benzyl-4-(2,5-difluorophenyl)-1H-pyrol-2-yl]-2,2-dimethylpropan-1-amine

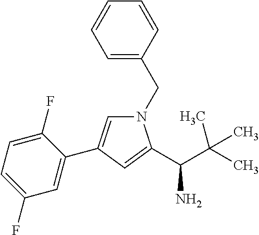 [SMILES: CC(C)(C)[C@@H](N)c1cc(-c2cc(F)ccc2F)cn1Cc1ccccc1]

10.00 g (49.01 mmol) of methyl 4-bromo-1H-pyrrole-2-carboxylate were initially charged in 100.0 ml of DMF, and 20.76 g (63.72 mmol) of caesium carbonate and 9.22 g (53.91 mmol) of benzyl bromide were added. The reaction mixture was stirred at RT overnight. The reaction mixture was partitioned between water and ethyl acetate and the aqueous phase was extracted with ethyl acetate. The combined organic phases were dried over magnesium sulphate and the solvent was evaporated under reduced pressure. The reaction was repeated with 90.0 g of methyl 4-bromo-1H-pyrrole-2-carboxylate.

The two combined reactions were purified by preparative RP-HPLC (column: Daiso 300×100; 10μ, flow rate: 250 ml/min, MeCN/water). The solvents were evaporated under reduced pressure and the residue was dried under high vacuum. This gave 125.15 g (87% of theory) of the compound methyl 1-benzyl-4-bromo-1H-pyrrole-2-carboxylate.

LC-MS (Method 1): Rₜ=1.18 min; MS (ESIpos): m/z=295 [M+H]⁺.

Under argon, 4.80 g (16.32 mmol) of methyl 1-benzyl-4-bromo-1H-pyrrole-2-carboxylate were initially charged in DMF, and 3.61 g (22.85 mmol) of (2,5-difluorophenyl)boronic acid, 19.20 ml of saturated sodium carbonate solution and 1.33 g (1.63 mmol) of [1,1′-bis(diphenylphosphino)ferrocene]-dichloropalladium(II) dichloromethane were added. The reaction mixture was stirred at 85° C. overnight. The reaction mixture was filtered through Celite and the filter cake was washed with ethyl acetate. The organic phase was extracted with water and then washed with saturated NaCl solution. The organic phase was dried over magnesium sulphate and the solvent was evaporated under reduced pressure. The residue was purified by silica gel chromatography (mobile phase: cyclohexane/ethyl acetate 100:3). The solvents were evaporated under reduced pressure and the residue was dried under high vacuum. This gave 3.60 g (67% of theory) of the compound methyl 1-benzyl-4-(2,5-difluorophenyl)-1H-pyrrole-2-carboxylate.

LC-MS (Method 7): Rₜ=1.59 min; MS (ESIpos): m/z=328 [M+H]⁺.

3.60 g (11.00 mmol) of methyl 1-benzyl-4-(2,5-difluorophenyl)-1H-pyrrole-2-carboxylate were initially charged in 90.0 ml of THF, and 1.04 g (27.50 mmol) of lithium aluminium hydride (2.4 M in THF) were added at 0° C. The reaction mixture was stirred at 0° C. for 30 minutes. At 0° C., saturated potassium sodium tartrate solution was added, and ethyl acetate was added to the reaction mixture. The organic phase was extracted three times with saturated potassium sodium tartrate solution. The organic phase was washed once with saturated NaCl solution and dried over magnesium sulphate. The solvent was evaporated under reduced pressure and the residue was dissolved in 30.0 ml of dichloromethane. 3.38 g (32.99 mmol) of manganese(IV) oxide were added, and the mixture was stirred at RT for 48 h. Another 2.20 g (21.47 mmol) of manganese(IV) oxide were added, and the mixture was stirred at RT overnight. The reaction mixture was filtered through Celite and the filter cake was washed with dichloromethane. The solvent was evaporated under reduced pressure and the residue 2.80 g of (l-benzyl-4-(2,5-difluorophenyl)-1H-pyrrole-2-carbaldehyde) was used without further purification in the next step of the synthesis.

LC-MS (Method 7): Rₜ=1.48 min; MS (ESIpos): m/z=298 [M+H]⁺.

28.21 g (94.88 mmol) of l-benzyl-4-(2,5-difluorophenyl)-1H-pyrrole-2-carbaldehyde together with 23.00 g (189.77 mmol) of (R)-2-methylpropane-2-sulphinamide were initially charged in 403.0 ml of absolute THF, and 67.42 g (237.21 mmol) of titanium(IV) isopropoxide were added and the mixture was stirred at RT overnight. 500.0 ml of saturated NaCl solution and 1000.0 ml of ethyl acetate were added, and the mixture was stirred at RT for 1 h. The mixture was filtered through kieselguhr and the filtrate was washed twice with saturated NaCl solution. The organic phase was dried over magnesium sulphate, the solvent was evaporated under reduced pressure and the residue was purified using Biotage Isolera (silica gel, column 1500+340 g SNAP, flow rate 200 ml/min, ethyl acetate/cyclohexane 1:10).

LC-MS (Method 7): Rₜ=1.63 min; MS (ESIpos): m/z=401 [M+H]⁺.

25.00 g (62.42 mmol) of (R)—N-{(E/Z)-[1-benzyl-4-(2,5-difluorophenyl)-1H-pyrrol-2-yl]methylene}-2-methylpropane-2-sulphinamide were initially charged in absolute THF under argon and cooled to −78° C. 12.00 g (187.27 mmol) of tert-butyllithium (1.7 M solution in pentane) were then added at −78° C. and the mixture was stirred at this temperature for 3 h. At −78° C., 71.4 ml of methanol and 214.3 ml of saturated ammonium chloride solution were then added in succession, and the reaction mixture was allowed to warm to RT and stirred at RT for 1 h. The mixture was diluted with ethyl acetate and washed with water. The organic phase was dried over magnesium sulphate and the solvent was evaporated under reduced pressure. The residue (R)—N-{(1R)-1-[1-benzyl-4-(2,5-difluorophenyl)-1H-pyrrol-2-yl]-2,2-dimethylpropyl}-2-methylpropane-2-sulphinamide was used without further purification in the next step of the synthesis.

LC-MS (Method 6): Rₜ=2.97 min; MS (ESIpos): m/z=459 [M+H]⁺.

28.00 g (61.05 mmol) of (R)—N-{(1R)-1-[1-benzyl-4-(2,5-difluorophenyl)-1H-pyrrol-2-yl]-2,2-dimethylpropyl}-2-methylpropane-2-sulphinamide were initially charged in 186.7 ml of 1,4-dioxane, and 45.8 ml of HCl in 1,4-dioxane solution (4.0 M) were then added. The reaction mixture was stirred at RT for 2 h and the solvent was evaporated under reduced pressure. The residue was purified by preparative RP-HPLC (column: Kinetix 100×30; flow rate: 60 ml/min, MeCN/water). The acetonitrile was evaporated under reduced pressure and dichloromethane was added to the aqueous residue. The organic phase was washed with sodium bicarbonate solution and dried over magnesium sulphate. The solvent was evaporated under reduced pressure and the residue was dried under high vacuum. This gave 16.2 g (75% of theory) of the title compound.

LC-MS (Method 6): Rₜ=2.10 min; MS (ESIpos): m/z=338 [M-NH₂]⁺, 709 [2M+H]⁺.

¹H-NMR (400 MHz, DMSO-d₆): δ [ppm]=0.87 (s, 9H), 1.53 (s, 2H), 3.59 (s, 1H), 5.24 (d, 2H), 6.56 (s, 1H), 6.94 (m, 1H), 7.10 (d, 2H), 7.20 (m, 1H), 7.26 (m, 2H), 7.34 (m, 2H), 7.46 (m, 1H).

### Intermediate C53

### (2S)-4-[{(1R)-1-[1-Benzyl-4-(2,5-difluorophenyl)-1H-pyrrol-2-yl]-2,2-dimethylpropyl}(glycoloyl)amino]-2-{[(9H-fluoren-9-ylmethoxy)carbonyl]amino}butanoic acid

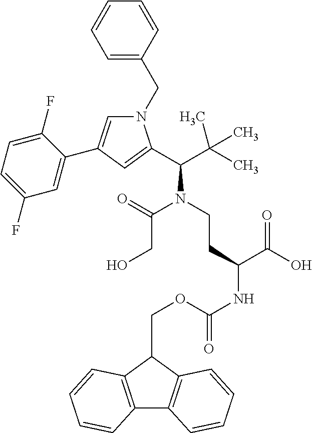 [SMILES: CC(C)(C)[C@H](c1cc(-c2cc(F)ccc2F)cn1Cc1ccccc1)N(CC[C@H](NC(=O)OCC1c2ccccc2-c2ccccc21)C(=O)O)C(=O)CO]

First, intermediate C52 was reductively alkylated with benzyl (2S)-2-{[(benzyloxy)carbonyl]amino}-4-oxobutanoate analogously to intermediate C58. The secondary amino group was then acylated with 2-chloro-2-oxoethyl acetate as described for Intermediate C58, and the two ester groups were then hydrolysed with 2M lithium hydroxide solution in methanol. The intermediate obtained in this manner was dissolved in ethanol, palladium on carbon (10%) was added and the mixture was hydrogenated at RT with hydrogen under standard pressure for 1 h. The deprotected compound was taken up in dioxane/water 2:1 and in the last step the Fmoc protective group was introduced using 9H-fluoren-9-ylmethyl chlorocarbonate in the presence of N,N-diisopropylethylamine.

LC-MS (Method 1): Rₜ=1.37 min; MS (ESIpos): m/z=734 (M−H)⁻.

### Intermediate C54

### N-[(2S)-4-[{(1R)-1-[1-Benzyl-4-(2,5-difluorophenyl)-1H-pyrrol-2-yl]-2,2-dimethylpropyl}(glycoloyl)amino]-2-{[(9H-fluoren-9-ylmethoxy)carbonyl]amino}butanoyl]-beta-alanine

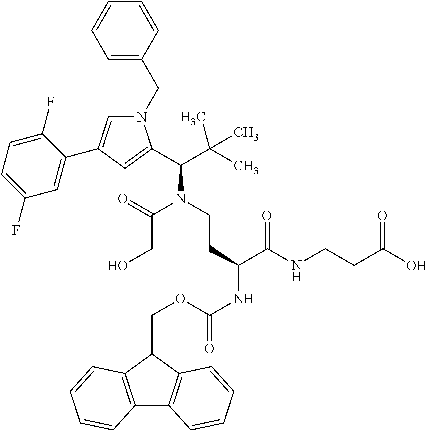 [SMILES: CC(C)(C)[C@H](c1cc(-c2cc(F)ccc2F)cn1Cc1ccccc1)N(CC[C@H](NC(=O)OCC1c2ccccc2-c2ccccc21)C(=O)NCCC(=O)O)C(=O)CO]

First, Intermediate C52 was reductively alkylated with benzyl N-[(2S)-2-{[(benzyloxy)carbonyl]amino}-4-oxobutanoyl]-beta-alaninate analogously to Intermediate C2. The secondary amino group was then acylated with 2-chloro-2-oxoethyl acetate as described for Intermediate C58. The intermediate obtained in this manner was dissolved in methanol, palladium on carbon (10%) was added and the mixture was hydrogenated at RT with hydrogen under standard pressure for 1 h. The ester group was then hydrolyzed with 2M lithium hydroxide solution in methanol. The deprotected compound was taken up in dioxane/water 2:1 and in the last step the Fmoc protective group was introduced using 9H-fluoren-9-ylmethyl chlorocarbonate in the presence of N,N-diisopropylethylamine. 48 mg of the title compound were obtained.

LC-MS (Method 1): Rₜ=1.38 min; MS (ESIpos): m/z=807 (M+H)⁺.

### Intermediate C58

### (2S)-4-[{(1R)-1-[1-Benzyl-4-(2,5-difluorophenyl)-1H-pyrrol-2-yl]-2,2-dimethylpropyl}(glycoloyl)amino]-2-({[2-(trimethylsilyl)ethoxy]carbonyl}amino)butanoic acid

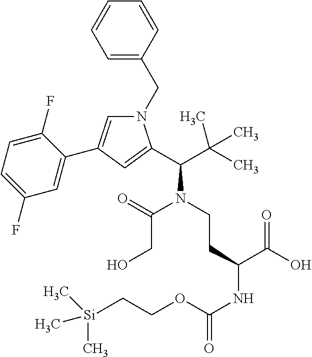 [SMILES: CC(C)(C)[C@H](c1cc(-c2cc(F)ccc2F)cn1Cc1ccccc1)N(CC[C@H](NC(=O)OCC[Si](C)(C)C)C(=O)O)C(=O)CO]

First, Intermediate C52 was reductively alkylated with benzyl (2S)-2-{[(benzyloxy)carbonyl]amino}-4-oxobutanoate analogously to Intermediate C2. The secondary amino group was then acylated with 2-chloro-2-oxoethyl acetate as described for Intermediate C27, and the two ester groups were then hydrolysed with 2M lithium hydroxide solution in methanol. The intermediate obtained in this manner was dissolved in ethanol, palladium on carbon (10%) was added and the mixture was hydrogenated at RT with hydrogen under standard pressure for 1 h.

500 mg (0.886 mmol) of this fully deprotected intermediate were taken up in 60 ml of dioxane, and 253 mg (0.975 mmol) of 1-({[2-(trimethylsilyl)ethoxy]carbonyl}oxy)pyrrolidine-2,5-dione and 198 μl of triethylamine were added. After 24 h of stirring at RT, the reaction was concentrated and the residue was purified by preparative HPLC. Combination of the appropriate fractions, concentration under reduced pressure and drying under high vacuum gave 312 mg (50% of theory) of the title compound.

LC-MS (Method 5): Rₜ=4.61 min; MS (ESIpos): m/z=658 (M+H)⁺.

Alternatively, Intermediate C58 was prepared by the following route:

4.3 g (12.2 mmol) of Intermediate C52 were dissolved in 525 ml of DCM, and 3.63 g (17.12 mmol) of sodium triacetoxyborohydride and 8.4 ml of acetic acid were added. After 5 min of stirring at RT, 8.99 g (24.5 mmol) of Intermediate L57 dissolved in 175 ml of DCM were added and the reaction was stirred at RT for a further 45 min. The reaction was then diluted with 300 ml of DCM and washed twice with 100 ml of sodium bicarbonate solution and once with saturated NaCl solution. The organic phase was dried over magnesium sulphate, the solvent was evaporated under reduced pressure and the residue was dried under high vacuum. The residue was then purified by preparative RP-HPLC (column: Chromatorex C18). After combination of the appropriate fractions, the solvent was evaporated under reduced pressure and the residue was dried under high vacuum. This gave 4.6 g (61% of theory) of methyl (2S)-4-({(1R)-1-[1-benzyl-4-(2,5-difluorophenyl)-1H-pyrrol-2-yl]-2,2-dimethylpropyl}amino)-2-({[2-(trimethylsilyl)ethoxy]carbonyl}aminobutanoate.

LC-MS (Method 12): Rₜ=1.97 min; MS (ESIpos): m/z=614 (M+H)⁺.

2.06 g (3.36 mmol) of this intermediate were initially charged in 76 ml of DCM and acylated with 0.81 ml (7.17 mmol) of 2-chloro-2-oxoethyl acetate in the presence of 2.1 ml of triethylamine. After 20 h of stirring at RT, 0.36 ml of 2-chlor-2-oxoethyl acetate and 0.94 ml of triethylamine were added and the reaction was stirred at RT for a further 15 min. The mixture was then diluted with 500 ml of ethyl acetate and extracted successively twice with 300 ml of 5% strength citric acid, twice with 300 ml of saturated sodium bicarbonate solution and once with 100 ml of saturated sodium chloride solution and then dried over magnesium sulphate and concentrated. Drying under high vacuum gave 2.17 g (79% of theory) of the protected intermediate.

LC-MS (Method 1): Rₜ=1.48 min; MS (ESIpos): m/z=714 (M+H)⁺.

2.17 mg (2.64 mmol) of this intermediate were dissolved in 54 ml of THF and 27 ml of water, and 26 ml of a 2-molar lithium hydroxide solution were added. The mixture was stirred at RT for 30 min and then adjusted to a pH between 3 and 4 using 1.4 ml of TFA. The mixture was concentrated under reduced pressure. Once most of the THF had been distilled off, the aqueous solution was extracted twice with DCM and then concentrated to dryness under reduced pressure. The residue was purified by preparative HPLC (column: Chromatorex C18). After combination of the appropriate fractions, the solvent was evaporated under reduced pressure and the residue was lyophilized from acetonitrile/water. This gave 1.1 g (63% of theory) of the title compound.

LC-MS (Method 1): Rₜ=1.34 min; MS (ESIpos): m/z=656 (M−H)⁻.

¹H-NMR (400 MHz, DMSO-d₆): δ [ppm]=0.03 (s, 9H), 0.58 (m, 1H), 0.74-0.92 (m, 11H), 1.40 (m, 1H), 3.3 (m, 2H), 3.7 (m, 1H), 3.8-4.0 (m, 2H), 4.15 (q, 2H), 4.9 and 5.2 (2d, 2H), 5.61 (s, 1H), 6.94 (m, 2H), 7.13-7.38 (m, 7H), 7.48 (s, 1H), 7.60 (m, 1H), 12.35 (s, 1H).

### Intermediate C59

### (2S)-4-({(1R)-1-[1-Benzyl-4-(2,5-difluorophenyl)-1H-pyrrol-2-yl]-2,2-dimethylpropyl}[(2S)-2-methoxypropanoyl]amino)-2-{[(9H-fluoren-9-ylmethoxy)carbonyl]amino}butanoic acid

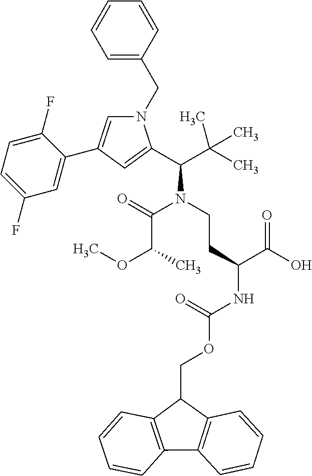 [SMILES: CO[C@@H](C)C(=O)N(CC[C@H](NC(=O)OCC1c2ccccc2-c2ccccc21)C(=O)O)[C@@H](c1cc(-c2cc(F)ccc2F)cn1Cc1ccccc1)C(C)(C)C]

Initially, the secondary amino group of benzyl (2S)-4-({(1R)-1-[1-benzyl-4-(2,5-difluorophenyl)-1H-pyrrol-2-yl]-2,2-dimethylpropyl}amino)-2-{[(benzyloxy)carbonyl]amino}butanoate was acylated with (2S)-2-methoxypropanoyl chloride (intermediate of Intermediate C53) in the presence of triethylamine as described for Intermediate C53. The intermediate obtained was taken up in ethanol, palladium on carbon (10%) was added and the mixture was hydrogenated at RT with hydrogen under standard pressure for 1 h. The deprotected compound was taken up in dioxane/water 2:1 and in the last step the Fmoc protective group was introduced using 9H-fluoren-9-ylmethyl chlorocarbonate in the presence of N,N-diisopropylethylamine.

LC-MS (Method 1): Rₜ=1.39 min; MS (ESIpos): m/z=764 (M−H)⁻.

### Intermediate C60

### (2S)-4-({(1R)-1-[1-Benzyl-4-(2,5-difluorophenyl)-1H-pyrrol-2-yl]-2,2-dimethylpropyl}[(2S)-2-methoxypropanoyl]amino)-2-{[(9H-fluoren-9-ylmethoxy)carbonyl]amino}butanoic acid

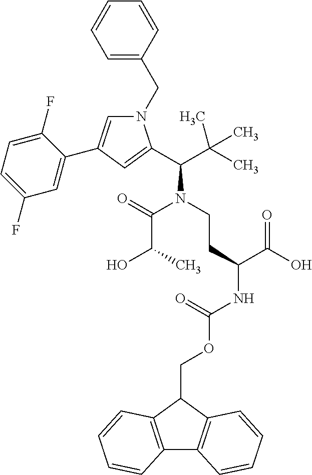 [SMILES: C[C@H](O)C(=O)N(CC[C@H](NC(=O)OCC1c2ccccc2-c2ccccc21)C(=O)O)[C@@H](c1cc(-c2cc(F)ccc2F)cn1Cc1ccccc1)C(C)(C)C]

The synthesis was carried out analogously to Intermediate C53.

LC-MS (Method 1): Rₜ=1.41 min; MS (ESIpos): m/z=750 (M+H)⁺.

### Intermediate C61

### N-[(2S)-4-[{(1R)-1-[1-Benzyl-4-(2,5-difluorophenyl)-1H-pyrrol-2-yl]-2,2-dimethylpropyl}(glycoloyl)amino]-2-({[2-(trimethylsilyl)ethoxy]carbonyl}amino)butanoyl]-beta-alanine

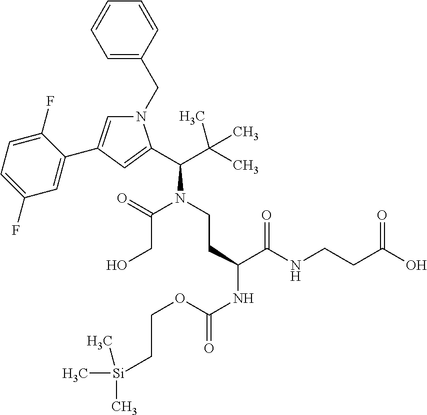 [SMILES: CC(C)(C)[C@H](c1cc(-c2cc(F)ccc2F)cn1Cc1ccccc1)N(CC[C@H](NC(=O)OCC[Si](C)(C)C)C(=O)NCCC(=O)O)C(=O)CO]

The title compound was prepared by coupling 60 mg (0.091 mmol) of Intermediate C58 with methyl β-alaninate, followed by ester cleavage with 2M lithium hydroxide solution. This gave 67 mg (61% of theory) of the title compound over 2 steps.

LC-MS (Method 1): Rₜ=1.29 min; MS (ESIpos): m/z=729 (M+H)⁺.

### Intermediate C62

### N-[(2S)-4-[{(1R)-1-[1-Benzyl-4-(2,5-difluorophenyl)-1H-pyrrol-2-yl]-2,2-dimethylpropyl}(glycoloyl)amino]-2-({[2-(trimethylsilyl)ethoxy]carbonyl}aminobutanoyl]-D-alanine

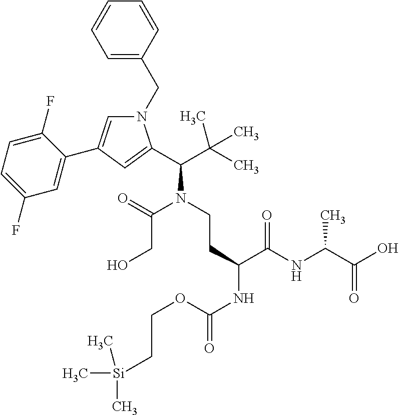 [SMILES: C[C@@H](NC(=O)[C@H](CCN(C(=O)CO)[C@@H](c1cc(-c2cc(F)ccc2F)cn1Cc1ccccc1)C(C)(C)C)NC(=O)OCC[Si](C)(C)C)C(=O)O]

The title compound was prepared analogously to Intermediate C61 from Intermediate C58 and methyl D-alaninate.

LC-MS (Method 1): Rₜ=1.32 min; MS (ESIpos): m/z=729 (M+H)⁺.

### Intermediate C64

### Trifluoroacetic acid/2-(trimethylsilyl)ethyl {(2S)-1-[(2-aminoethyl)amino]-4-[{(1R)-1-[1-benzyl-4-(2,5-difluorophenyl)-1H-pyrrol-2-yl]-2,2-dimethylpropyl}(glycoloyl)amino]-1-oxobutan-2-yl}-carbamate (1:1)

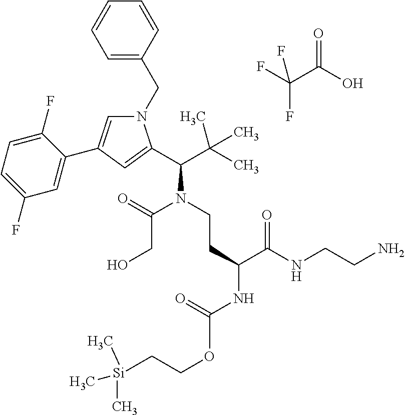 [SMILES: CC(C)(C)[C@H](c1cc(-c2cc(F)ccc2F)cn1Cc1ccccc1)N(CC[C@H](NC(=O)OCC[Si](C)(C)C)C(=O)NCCN)C(=O)CO ; O=C(O)C(F)(F)F]

The title compound was prepared from Intermediate C58 analogously to Intermediate C63.

HPLC (Method 11): Rₜ=2.4 min;

LC-MS (Method 1): Rₜ=1.01 min; MS (ESIpos): m/z=700 (M+H)⁺.

### Intermediate C65

### (8S)-8-{2-[{(1R)-1-[1-Benzyl-4-(2,5-difluorophenyl)-1H-pyrrol-2-yl]-2,2-dimethylpropyl}-(glycoloyl)amino]ethyl}-2,2-dimethyl-6,11-dioxo-5-oxa-7,10-diaza-2-silatetradecan-14-oic acid

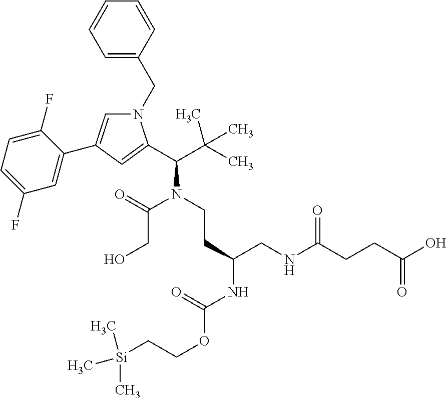 [SMILES: CC(C)(C)[C@H](c1cc(-c2cc(F)ccc2F)cn1Cc1ccccc1)N(CC[C@@H](CNC(=O)CCC(=O)O)NC(=O)OCC[Si](C)(C)C)C(=O)CO]

215 mg (0.59 mmol) of Intermediate L66 were initially charged in 25 ml of dichloromethane, and 377 mg (0.89 mmol) of Dess-Martin periodinane and 144 μl (1.78 mmol) of pyridine were added. The mixture was stirred at RT for 30 min. The reaction was then diluted with 300 ml of dichloromethane and the organic phase was washed in each case twice with 10% strength Na₂S₂O₃ solution, 10% strength citric acid solution and saturated sodium bicarbonate solution. The organic phase was dried over magnesium sulphate and the solvent was evaporated under reduced pressure. This gave 305 mg of the aldehyde which was reacted without further purification. 175 mg (0.49 mmol) of Intermediate C52 were dissolved in 50 ml of dichloromethane, and 147 mg (0.69 mmol) of sodium triacetoxyborohydride and 32.5 μl of acetic acid were added. After 5 min of stirring at RT, 214 mg (0.593 mmol) of the aldehyde described above were added, and the reaction was stirred at RT overnight. Here, instead of the expected product, 2-(trimethylsilyl)ethyl [(2S)-4-({(1R)-1-[1-benzyl-4-(2,5-difluorophenyl)-1H-pyrrol-2-yl]-2,2-dimethylpropyl}amino)-1-(2,5-dioxopyrrolidin-1-yl)butan-2-yl]carbamate was formed. Since this imide can also be converted into the title compound, the reaction was concentrated and the residue was purified by preparative HPLC. After combination of the appropriate imide-containing fractions, the solvent was evaporated under reduced pressure and the residue was dried under high vacuum. This gave 195 mg (58%) of the imide named above.

LC-MS (Method 5): Rₜ=3.32 min; MS (ESIpos): m/z=667 (M+H)⁺.

65 mg (97.5 μmol) of this imide were taken up in 15 ml of dichloromethane, and 367 μl (3.4 mmol) of acetoxyacetyl chloride and 595 μl of N,N-diisopropylethylamine were added. After 30 min of stirring at RT, the reaction was concentrated without heating under reduced pressure and the residue was purified by preparative HPLC. The appropriate fractions were combined giving, after evaporation of the solvents and drying under high vacuum, 28 mg (37% of theory) of (8S)-11-{(1R)-1-[1-benzyl-4-(2,5-difluorophenyl)-1H-pyrrol-2-yl]-2,2-dimethylpropyl}-8-[(2,5-dioxopyrrolidin-1-yl)methyl]-2,2-dimethyl-6,12-dioxo-5-oxa-7,11-diaza-2-silatridecan-13-yl acetate.

LC-MS (Method 1): Rₜ=1.44 min; MS (ESIpos): m/z=767 (M+H)⁺.

28 mg (37 μmol) of this intermediate were dissolved in 3 ml of methanol, and 548 μl of a 2M lithium hydroxide solution were added. After 10 min of stirring at RT, the reaction was adjusted to pH 4 with trifluoroacetic acid and then concentrated. The residue was purified by preparative HPLC. The appropriate fractions were combined, the solvent was evaporated and the residue was dried under high vacuum, giving 26 mg (96% of theory) of the title compound as a white solid.

LC-MS (Method 1): Rₜ=1.33 min; MS (ESIpos): m/z=743 (M+H)⁺.

### Intermediate C66

### 2-(Trimethylsilyl)ethyl [(2S)-4-[{(1R)-1-[1-benzyl-4-(2,5-difluorophenyl)-1H-pyrrol-2-yl]-2,2-dimethylpropyl}(glycoloyl)amino]-1-{[2-(glycylamino)ethyl]amino}-1-oxobutan-2-yl]carbamate

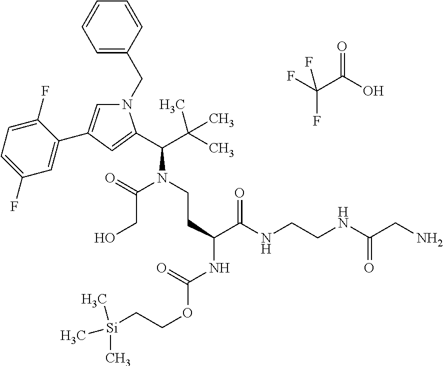 [SMILES: CC(C)(C)[C@H](c1cc(-c2cc(F)ccc2F)cn1Cc1ccccc1)N(CC[C@H](NC(=O)OCC[Si](C)(C)C)C(=O)NCCNC(=O)CN)C(=O)CO ; O=C(O)C(F)(F)F]

First, trifluoroacetic acid/benzyl {2-[(2-aminoethyl)amino]-2-oxoethyl}carbamate (1:1) was prepared from N-[(benzyloxy)carbonyl]glycine and tert-butyl (2-aminoethyl) carbamate according to classical methods of peptide chemistry (HATU coupling and Boc removal).

13 mg (0.036 mmol) of this intermediate and 25 mg (0.033 mmol) of Intermediate C58 were taken up in 3 ml of DMF, and 19 mg (0.05 mmol) of HATU and 17 μl of N,N-diisopropylethylamine were added. After 10 min of stirring at RT, the mixture was concentrated and the residue was purified by preparative HPLC. This gave 17.8 mg (60% of theory) of the intermediate.

LC-MS (Method 1): Rₜ=1.36 min; MS (ESIpos): m/z=891 (M+H)⁺.

17 mg (0.019 mmol) of this intermediate were dissolved in 10 ml of ethanol, palladium on carbon (10%) was added and the mixture was hydrogenated at RT with hydrogen at standard pressure for 2 h. The catalyst was filtered off, the solvents were evaporated under reduced pressure and the residue was dried under high vacuum. This gave 9 mg (62% of theory) of the title compound.

LC-MS (Method 1): Rₜ=1.03 min; MS (ESIpos): m/z=757 (M+H)⁺.

### Intermediate C67

### 9H-Fluoren-9-ylmethyl [3-({(1R)-1-[1-benzyl-4-(2,5-difluorophenyl)-1H-pyrrol-2-yl]-2,2-dimethylpropyl}amino)propyl]carbamate

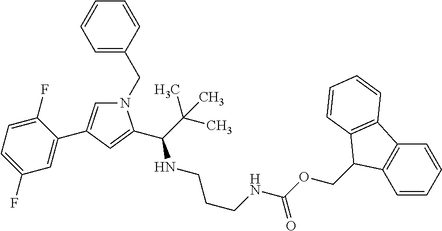 [SMILES: CC(C)(C)[C@@H](NCCCNC(=O)OCC1c2ccccc2-c2ccccc21)c1cc(-c2cc(F)ccc2F)cn1Cc1ccccc1]

605.3 mg (1.71 mmol) of (1R)-1-[1-benzyl-4-(2,5-difluorophenyl)-1H-pyrrol-2-yl]-2,2-dimethylpropan-1-amine (Intermediate C52) were initially charged in 10.0 ml of dichloromethane, and 506.7 mg (2.39 mmol) of sodium triacetoxyborohydride and 117.9 mg (1.96 mmol) of acetic acid were added and the mixture was stirred at RT for 5 min. 580.0 mg (1.96 mmol) of 9H-fluoren-9-ylmethyl (3-oxopropyl)carbamate (Intermediate L70) dissolved in 10.0 ml of dichloromethane were added and the reaction mixture stirred at RT overnight. The reaction mixture was diluted with ethyl acetate and the organic phase was washed in each case twice with saturated sodium carbonate solution and saturated NaCl solution. The organic phase was dried over magnesium sulphate and the solvent was evaporated under reduced pressure. The residue was purified by silica gel chromatography (mobile phase: cyclohexane/ethyl acetate 3:1). The solvents were evaporated under reduced pressure and the residue was dried under high vacuum. This gave 514.7 mg (46% of theory) of the title compound.

LC-MS (Method 1): Rₜ=1.10 min; MS (ESIpos): m/z=634 (M+H)⁺.

### Intermediate C69

### 11-{(1R)-1-[1-Benzyl-4-(2,5-difluorophenyl)-1H-pyrrol-2-yl]-2,2-dimethylpropyl}-2,2-dimethyl-6,12-dioxo-5-oxa-14-thia-7,11-diaza-2-silaheptadecan-17-oic acid

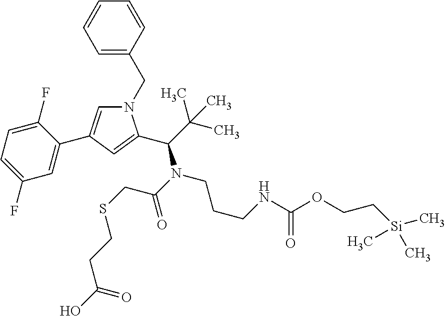 [SMILES: CC(C)(C)[C@H](c1cc(-c2cc(F)ccc2F)cn1Cc1ccccc1)N(CCCNC(=O)OCC[Si](C)(C)C)C(=O)CSCCC(=O)O]

117.0 mg (0.19 mmol) of (2-(trimethylsilylethyl {3-[{(1R)-1-[1-benzyl-4-(2,5-difluorophenyl)-1H-pyrrol-2-yl]-2,2-dimethylpropyl}(chloroacetyl)amino]propyl}carbamate (Intermediate C70) and 21.6 mg (0.20 mmol) of 3-sulphanylpropanoic acid were initially charged in 3.0 ml of methanol, 89.5 mg (0.65 mmol) of potassium carbonate were added and the mixture was stirred at 50° C. for 4 h. The reaction mixture was diluted with ethyl acetate and the organic phase was washed with water and saturated NaCl solution. The organic phase was dried over magnesium sulphate, the solvent was evaporated under reduced pressure and the residue was dried under high vacuum. The residue was used without further purification in the next step of the synthesis. This gave 106.1 mg (73% of theory) of the title compound.

LC-MS (Method 1): Rₜ=1.42 min; MS (ESIneg): m/z=700 (M−H)⁻.

### Intermediate C70

### (2-(Trimethylsilyl)ethyl {3-[{(1R)-1-[1-benzyl-4-(2,5-difluorophenyl)-1H-pyrrol-2-yl]-2,2-dimethylpropyl}(chloroacetyl)amino]propyl}carbamate

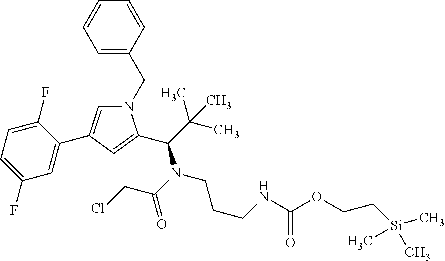 [SMILES: CC(C)(C)[C@H](c1cc(-c2cc(F)ccc2F)cn1Cc1ccccc1)N(CCCNC(=O)OCC[Si](C)(C)C)C(=O)CCl]

908.1 mg (1.63 mmol) of 2-(trimethylsilyl)ethyl [3-({(1R)-1-[1-benzyl-4-(2,5-difluorophenyl)-1H-pyrrol-2-yl]-2,2-dimethylpropyl}amino)propyl]carbamate (see synthesis of Intermediate C11) and 545.6 mg (5.39 mmol) of triethylamine were initially charged in 10.0 ml of dichloromethane, and the mixture was cooled to 0° C. At this temperature, 590.5 mg (5.23 mmol) of chloroacetyl chloride were added and the mixture was stirred at RT overnight. The reaction mixture was diluted with ethyl acetate and the organic phase was washed in each case three times with saturated sodium bicarbonate solution and saturated ammonium chloride solution. The organic phase was washed with saturated NaCl solution and dried over magnesium sulphate. The residue was purified by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 673.8 mg (65% of theory) of the title compound.

LC-MS (Method 1): Rₜ=1.53 min; MS (ESIneg): m/z=676 (M+HCOO⁻)⁻.

### Intermediate C71

### S-(11-{(1R)-1-[1-Benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine/trifluoroacetic acid (1:1)

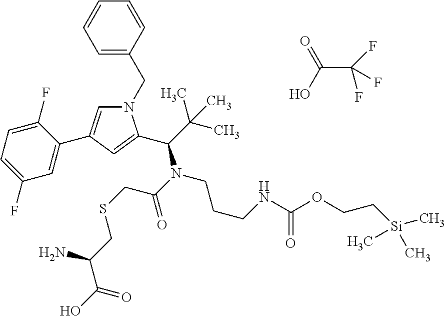 [SMILES: CC(C)(C)[C@H](c1cc(-c2cc(F)ccc2F)cn1Cc1ccccc1)N(CCCNC(=O)OCC[Si](C)(C)C)C(=O)CSC[C@H](N)C(=O)O ; O=C(O)C(F)(F)F]

536.6 mg (4.43 mmol) of L-cysteine were suspended in 2.5 ml of water together with 531.5 mg (6.33 mmol) of sodium bicarbonate. 400.0 mg (0.63 mmol) of 2-(trimethylsilyl)ethyl {3-[{(1R)-1-[l-benzyl-4-(2,5-difluorophenyl)-1H-pyrrol-2-yl]-2,2-dimethylpropyl}(chloroacetyl)amino]propyl}carbamate (Intermediate C70) dissolved in 25.0 ml of isopropanol and 1.16 g (7.59 mmol) of 1,8-diazabicyclo[5.4.0]undec-7-ene were added. The reaction mixture was stirred at 50° C. for 1.5 h. Ethyl acetate was added to the reaction mixture and the organic phase was washed repeatedly with saturated sodium bicarbonate solution and once with sat. NaCl solution. The organic phase was dried over magnesium sulphate, the solvent was evaporated under reduced pressure and the residue was dried under high vacuum. The residue was purified by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 449.5 mg (86% of theory) of the title compound.

LC-MS (Method 1): Rₜ=1.20 min; MS (ESIpos): m/z=717 (M+H)⁺.

### Intermediate C72

### (9S)-9-{[{(1R)-1-[1-Benzyl-4-(2,5-difluorophenyl)-1H-pyrrol-2-yl]-2,2-dimethylpropyl}(glycoloyl)amino]methyl}-2,2-dimethyl-6,11-dioxo-5-oxa-7,10-diaza-2-silatetradecan-14-oic acid

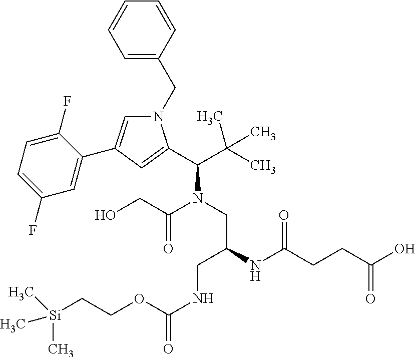 [SMILES: CC(C)(C)[C@H](c1cc(-c2cc(F)ccc2F)cn1Cc1ccccc1)N(C[C@H](CNC(=O)OCC[Si](C)(C)C)NC(=O)CCC(=O)O)C(=O)CO]

90 mg (0.212 mmol) of Intermediate L72 were initially charged in 6 ml of dichloromethane, and 86 μl (1.06 mmol) of pyridine and 135 mg (0.318 mmol) of Dess-Martin periodinane were added. The mixture was stirred at RT for 30 min. The reaction was then diluted with 30 ml of dichloromethane and the organic phase was washed twice with 10% strength Na₂S₂O₃ solution and once with 5% strength citric acid solution. The organic phase was dried over magnesium sulphate and the solvent was evaporated under reduced pressure. The aldehyde obtained in this manner was reacted without further purification.

63 mg (0.177 mmol) of Intermediate C52 were dissolved in 15 ml of dichloromethane, and 52.4 mg (0.247 mmol) of sodium triacetoxyborohydride and 20.2 μl of acetic acid were added. After 5 min of stirring at RT, 89.6 mg (0.212 mmol) of the aldehyde described above were added, and the reaction was stirred at RT for 20 min. The reaction was concentrated under reduced pressure and the residue was purified by preparative HPLC. After combination of the appropriate fractions, the solvent was evaporated under reduced pressure and the residue was lyophilized from acetonitrile/water. This gave 71 mg (53% of theory over 2 steps) of benzyl (9R)-9-[({(1R)-1-[1-benzyl-4-(2,5-difluorophenyl)-1H-pyrrol-2-yl]-2,2-dimethylpropyl}amino)methyl]-2,2-dimethyl-6,11-dioxo-5-oxa-7,10-diaza-2-silatetradecan-14-oate.

LC-MS (Method 1): Rₜ=1.21 min; MS (ESIpos): m/z=761 (M+H)⁺.

70 mg (92 μmol) of this intermediate were taken up in 15 ml of dichloromethane, the mixture was cooled to 10° C. and 54 μl of triethylamine and 25.5 μl (0.23 mmol) of acetoxyacetyl chloride were added. After 1 h of stirring at RT, the same amounts of acid chloride and triethylamine were added, and once more after a further hour of stirring at RT. The reaction was then stirred at RT for a further 30 min and then concentrated under reduced pressure, and the residue was purified by preparative HPLC. The appropriate fractions were combined giving, after evaporation of the solvents and lyophilization of the residue from acetonitrile/water, 46.5 mg (59% of theory) of the acylated intermediate.

LC-MS (Method 1): Rₜ=1.53 min; MS (ESIpos): m/z=861 (M+H)⁺.

46 mg (53 μmol) of this intermediate were dissolved in 5 ml of methanol, and 2.7 ml of a 2M lithium hydroxide solution were added. After 10 min of stirring at RT, the reaction was adjusted to pH 3-4 with acetic acid and then diluted with 15 ml of water. The aqueous phase was extracted with ethyl acetate and the organic phase was dried over magnesium sulphate and concentrated. The residue was lyophilized from acetonitrile/water giving, after drying of the residue under high vacuum, 37 mg (90% of theory) of the title compound as a white solid.

LC-MS (Method 1): Rₜ=1.32 min; MS (ESIpos): m/z=729 (M+H)⁺.

### Intermediate C73

### S-(11-{(1R)-1-[1-Benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[3-(trimethylsilyl)propanoyl]-L-cysteine

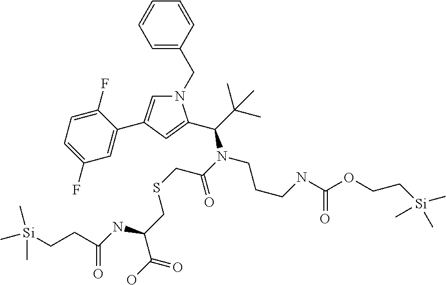

619 mg (0.86 mmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine/trifluoroacetic acid (1:1) (Intermediate C71) were initially charged in 8.8 ml of dichloromethane, and 87 mg (0.86 mmol) of triethylamine and 224 mg (0.86 mmol) of N-[2-(trimethylsilyl)ethoxycarbonyloxy]pyrrolidine-2,5-dione were added. After 1 h, 45 mg (0.17 mmol) of N-[2-(trimethylsilyl)ethoxycarbonyloxy]pyrrolidine-2,5-dione were added. The reaction mixture was stirred at RT for 1 h. The mixture was concentrated under reduced pressure, the residue was taken up in dichloromethane and the organic phase was then washed twice with water and a saturated sodium bicarbonate solution. The organic phase was dried over magnesium sulphate, concentrated on a rotary evaporator and dried under high vacuum. The residue was used further without further purification. This gave 602 mg (71%, purity 87%) of the title compound.

LC-MS (Method 1): Rₜ=1.58 min; MS (ESIpos): m/z=861 (M+H)⁺.

### Intermediate C74

### Trifluoroacetic acid 2-(trimethylsilyl)ethyl 3-amino-N-[(2S)-4-[{(1R)-1-[1-benzyl-4-(2,5-difluorophenyl)-1H-pyrrol-2-yl]-2,2-dimethylpropyl}(glycoloyl)amino]-2-({[2-(trimethylsilyl)ethoxy]carbonyl}amino)butanoyl]-D-alaninate (1:1)

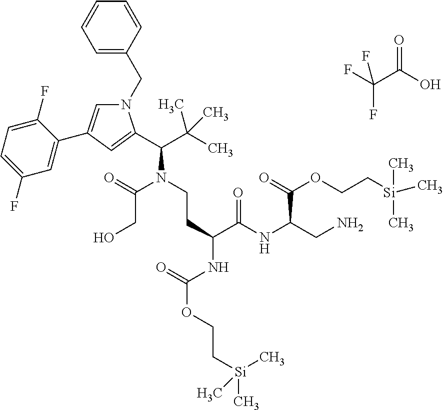 [SMILES: CC(C)(C)[C@H](c1cc(-c2cc(F)ccc2F)cn1Cc1ccccc1)N(CC[C@H](NC(=O)OCC[Si](C)(C)C)C(=O)N[C@H](CN)C(=O)OCC[Si](C)(C)C)C(=O)CO ; O=C(O)C(F)(F)F]

75 mg (0.114 mmol) of Intermediate C58 were taken up in 12.5 ml of DMF and coupled with 78 mg (0.171 mmol) of Intermediate L75 in the presence of 65 mg (0.11 mmol) of HATU and 79 μl of N,N-diisopropylethylamine. After purification by preparative HPLC, the intermediate was taken up in 20 ml of ethanol and hydrogenated over 10% palladium on activated carbon at RT under hydrogen standard pressure for 1 h. The catalyst was then filtered off, the solvent was removed under reduced pressure and the product was purified by preparative HPLC. Lyophilization from acetonitrile/water 1:1 gave 63 mg (64% of theory over 2 steps) of the title compound.

LC-MS (Method 1): Rₜ=1.16 min; MS (EIpos): m/z=844 [M+H]⁺.

### Intermediate C75

### Methyl (2S)-4-[(acetoxyacetyl) {(1R)-1-[1-benzyl-4-(2,5-difluorophenyl)-1H-pyrrol-2-yl]-2,2-dimethylpropyl}amino]-2-({[2-(trimethylsilyl)ethoxy]carbonyl}amino)butanoate

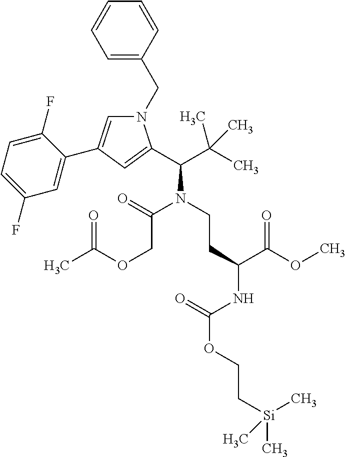 [SMILES: COC(=O)[C@H](CCN(C(=O)COC(C)=O)[C@@H](c1cc(-c2cc(F)ccc2F)cn1Cc1ccccc1)C(C)(C)C)NC(=O)OCC[Si](C)(C)C]

4.3 g (12.2 mmol) of Intermediate C52 were dissolved in 525 ml of DCM, and 3.63 g (17.12 mmol) of sodium triacetoxyborohydride and 8.4 ml of acetic acid were added. After 5 min of stirring at RT, 3.23 g (11.85 mmol) of methyl (2S)-4-oxo-2-({[2-(trimethylsilyl)ethoxy]carbonyl}aminobutanoate (prepared from (3S)-3-amino-4-methoxy-4-oxobutanoic acid by classical methods) dissolved in 175 ml of DCM were added, and the mixture was stirred at RT for a further 45 min. The mixture was then diluted with DCM and extracted twice with 100 ml of saturated sodium bicarbonate solution and then with saturated sodium chloride solution. The organic phase was dried over magnesium sulphate, filtered and concentrated. The residue was purified by preparative HPLC. Combination of the appropriate fractions, concentration and drying of the residue under high vacuum gave 4.6 g (61% of theory) of the intermediate.

LC-MS (Method 12): Rₜ=1.97 min; MS (ESIpos): m/z=614.32 (M+H)⁺.

200 mg (0.33 mmol) of this intermediate were dissolved in 10 ml of DCM, and 105 μl of triethylamine and 77 μl (0.717 mmol) of acetoxyacetyl chloride were then added. The mixture was stirred at RT overnight and then concentrated under reduced pressure. The residue was taken up in ethyl acetate and extracted twice with saturated sodium bicarbonate solution and then with saturated sodium chloride solution. The organic phase was dried over magnesium sulphate and then concentrated. This gave 213 mg (75%) of the title compound as a beige foam.

LC-MS (Method 1): Rₜ=1.46 min; MS (ESIpos): m/z=714 (M+H)⁺.

### Intermediate C76

### N-[(Benzyloxy)carbonyl]-L-valyl-N-{(1S)-3-[{(1R)-1-[1-benzyl-4-(2,5-difluorophenyl)-1H-pyrrol-2-yl]-2,2-dimethylpropyl}(glycoloyl)amino]-1-carboxypropyl}-L-alaninamide

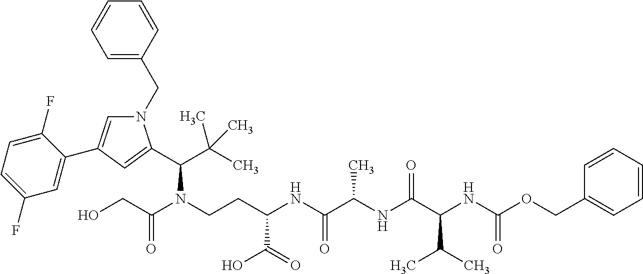 [SMILES: CC(C)[C@H](NC(=O)OCc1ccccc1)C(=O)N[C@@H](C)C(=O)N[C@@H](CCN(C(=O)CO)[C@@H](c1cc(-c2cc(F)ccc2F)cn1Cc1ccccc1)C(C)(C)C)C(=O)O]

The title compound was prepared from Intermediate C75 according to classical methods of peptide chemistry (removal of the Teoc protective group with zinc chloride, acylation with N-[(benzyloxy)carbonyl]-L-valyl-L-alanine in the presence of HATU and ester cleavage with lithium hydroxide in THF/water).

LC-MS (Method 1): Rₜ=1.23 min; MS (ESIpos): m/z=818 (M+H)⁺.

### Intermediate C77

### S-(11-{(1R)-1-[1-Benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-(4-tert-butoxy-4-oxobutanoyl)-L-cysteine

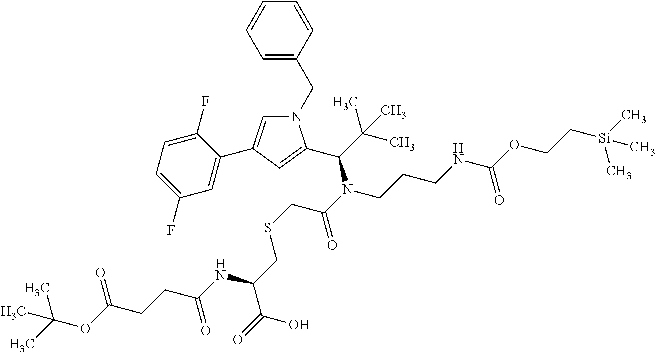 [SMILES: CC(C)(C)OC(=O)CCC(=O)N[C@@H](CSCC(=O)N(CCCNC(=O)OCC[Si](C)(C)C)[C@@H](c1cc(-c2cc(F)ccc2F)cn1Cc1ccccc1)C(C)(C)C)C(=O)O]

4-tert-Butoxy-4-oxobutanoic acid (8.39 mg, 48.1 μmol) was initially charged in 1.0 ml of DMF, 7.37 mg (48.1 μmol) of 1-hydroxy-1H-benzotriazole hydrate, 15.5 mg ((48.1 μmol) of (benzotriazol-1-yloxy)bisdimethylaminomethylium fluoroborat and 8.60 μl (48.1 μmol) of N,N-diisopropylethylamine were added and the mixture was stirred at RT for 10 minutes. 40.0 mg (0.048 mmol) S-(11-{(1R)-1-[1-Benzyl-4-(2,5-difluorphenyl)-1H-pyrrol-2-yl]-2,2-dimethylpropyl}-2,2-dimethyl-6,12-dioxo-5-oxa-7,11-diaza-2-silatridecan-13-yl)-L-cysteine trifluoroacetic acid (1:1) (Intermediate C71) were initially charged in 1.0 ml of DMF, 25.4 μl (141.9 μmol) of N,N-diisopropylethylamine were added, the mixture was added to the reaction and the reaction mixture was stirred at RT for 4 h. The reaction mixture was purified directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 35.0 mg (83% of theory) of the title compound.

LC-MS (Method 12): Rₜ=2.76 min; MS (ESIpos): m/z=873 [M+H]⁺

### Intermediate C78

### 11-{(1R)-1-[1-Benzyl-4-(2,5-difluorophenyl)-1H-pyrrol-2-yl]-2,2-dimethylpropyl}-2,2-dimethyl-6,12-dioxo-5-oxa-7,11-diaza-2-silapentadecane-15-acid

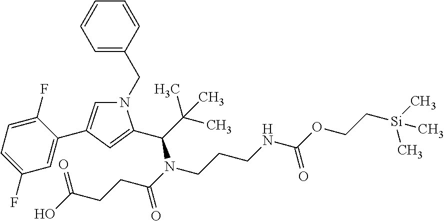 [SMILES: CC(C)(C)[C@H](c1cc(-c2cc(F)ccc2F)cn1Cc1ccccc1)N(CCCNC(=O)OCC[Si](C)(C)C)C(=O)CCC(=O)O]

197 mg (0.354 mmol) of 2-(trimethylsilyl)ethyl [3-({(1R)-1-[1-benzyl-4-(2,5-difluorophenyl)-1H-pyrrol-2-yl]-2,2-dimethylpropyl}amino)propyl]carbamate (see synthesis of Intermediate C11) were initially charged in 5.0 ml of dichloromethane, and the mixture was heated to 40° C. At this temperature, 240 μl (3.0 mmol) of pyridine and 220 μl (1.8 mmol) of methyl 4-chloro-4-oxobutanoate were added, and the mixture was stirred at RT for 1 h. 240 μl (3.0 mmol) of pyridine and 220 μl (1.8 mmol) of methyl 4-chloro-4-oxobutanoate were then added, and the mixture was stirred at RT for 1 h. 240 μl (3.0 mmol) of pyridine and 220 μl (1.8 mmol) of methyl 4-chloro-4-oxobutanoate were then added, and the mixture was stirred at RT for 1 h. The reaction mixture was diluted with ethyl acetate and the organic phase was extracted in each case three times with 5% strength KHSO₄ solution. The organic phase was washed with saturated NaCl solution and dried over magnesium sulphate. The solvents were evaporated under reduced pressure. The residue was purified by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 74.1 mg (31% of theory) of methyl 11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silapentadecan-15-oate.

LC-MS (Method 1): Rₜ=1.49 min; MS (ESIpos): m/z=670 [M+H]⁺

78.3 mg (117 μmol) of methyl 11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silapentadecan-15-oate were initially charged in 4.0 ml of THF, and 800 μl of methanol, 160 μl of water and 230 μl (230 μmol) of aqueous LiOH solution (1M) were added. The reaction mixture was stirred at RT for 3 h, quenched with acetic acid and purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 64.8 mg (85% of theory) of the title compound.

LC-MS (Method 12): Rₜ=2.61 min; MS (ESIneg): m/z=654 [M−H]⁻

### Intermediate C79

### Trifluoroacetic acid 2-(trimethylsilyl)ethyl 3-amino-N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-D-alaninate (1:1)

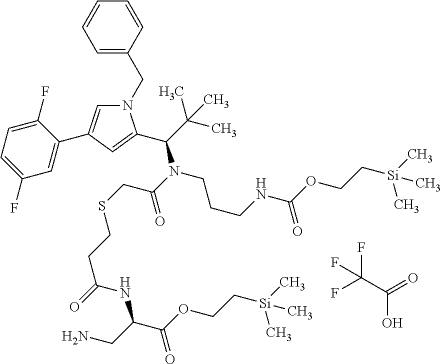 [SMILES: CC(C)(C)[C@H](c1cc(-c2cc(F)ccc2F)cn1Cc1ccccc1)N(CCCNC(=O)OCC[Si](C)(C)C)C(=O)CSCCC(=O)N[C@H](CN)C(=O)OCC[Si](C)(C)C ; O=C(O)C(F)(F)F]

57.4 mg (81.8 μmol) of 11-{(1R)-1-[1-benzyl-4-(2,5-difluorophenyl)-1H-pyrrol-2-yl]-2,2-dimethylpropyl}-2,2-dimethyl-6,12-dioxo-5-oxa-14-thia-7,11-diaza-2-silaheptadecan-17-oic acid (Intermediate C69) were initially charged in 5.7 ml of DMF, 74.0 mg (164 μmol) of trifluoroacetic acid 2-(trimethylsilyl)ethyl 3-{[(benzyloxy)carbonyl]amino}-D-alaninate (1:1) (Intermediate L75), 43 μl (250 μmol) of N,N-diisopropylethylamine and 62.2 mg (164 μmol) of HATU were added and the mixture was stirred at RT for 1 h. The reaction mixture was stirred at RT for 1 h, quenched with acetic acid and purified directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 52.4 mg (63% of theory) of the compound 2-(trimethylsilyl)ethyl N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-3-{[(benzyloxy)carbonyl]amino}-D-alaninate.

LC-MS (Method 1): Rₜ=1.64 min; MS (ESIpos): m/z=1022 [M]⁺

Under argon, 6.23 mg (27.7 μmol) of palladium(II) acetate: were initially charged in 3.0 ml of dichloromethane, 12 μl (83 μmol) of triethylamine and 89 μl (550 μmol) of triethylsilane were added and the mixture was stirred for 5 minutes. 56.7 mg (55.5 μmol) of 2-(trimethylsilyl)ethyl N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-3-{[(benzyloxy)carbonyl]amino}-D-alaninate in 3.0 ml of dichloromethane were then added, and the mixture was stirred at RT overnight. The mixture was concentrated almost to dryness, acetonitrile/water was added, and the mixture was filtered and purified by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 37.4 mg (67% of theory) of the title compound.

LC-MS (Method 12): Rₜ=2.15 min; MS (ESIpos): m/z=888 [M+H]⁺

### Intermediate C80

### S-(11-{(1R)-1-[1-Benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[15-(glycylamino)-4,7,10,13-tetraoxapentadecan-1-oyl]-L-cysteine trifluoroacetic acid (1:1)

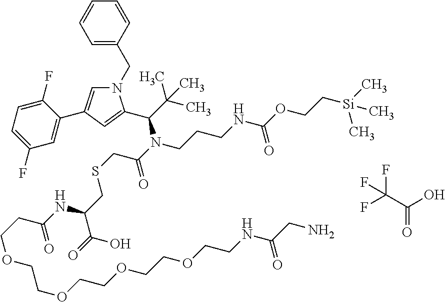 [SMILES: CC(C)(C)[C@H](c1cc(-c2cc(F)ccc2F)cn1Cc1ccccc1)N(CCCNC(=O)OCC[Si](C)(C)C)C(=O)CSC[C@H](NC(=O)CCOCCOCCOCCOCCNC(=O)CN)C(=O)O ; O=C(O)C(F)(F)F]

Under argon, 43.4 mg (95.1 μmol) of 1-({N-[(benzyloxy)carbonyl]glycyl}amino)-3,6,9,12-tetraoxapentadecan-15-oic acid (Intermediate L90) were initially charged in 2.5 ml of DMF, 14.6 mg (95.1 μmol) of 1-hydroxy-1H-benzotriazole hydrate, 30.5 mg (95.1 μmol) of (benzotriazol-1-yloxy)bisdimethylaminomethylium fluoroborate and 16.5 μl (95.1 μmol) of N,N-diisopropylethylamine were added and the mixture was stirred for 10 min. 79.0 mg (95.1 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine trifluoroacetic acid (1:1) (Intermediate C71) were dissolved in 2.5 ml of DMF, 49.5 μl (285.3 μmol) of N,N-diisopropylethylamine were added and the mixture was added to the reaction. The reaction mixture was stirred at RT for 2 h and purified directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 44.2 mg (40% of theory) of the compound S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[15-({N-[(benzyloxy)carbonyl]glycyl}amino)-4,7,10,13-tetraoxapentadecan-1-oyl]-L-cysteine.

LC-MS (Method 12): Rₜ=2.57 min; MS (ESIpos): m/z=1156 [M+H]⁺

60.2 mg (52.1 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[15-({N-[(benzyloxy)carbonyl]glycyl}amino)-4,7,10,13-tetraoxapentadecan-1-oyl]-L-cysteine were suspended in 3.0 ml of ethanol, 6.0 mg of palladium on activated carbon (10%) were added and the mixture was hydrogenated with hydrogen at RT and standard pressure for 1 h. Twice, 6.0 mg of palladium on activated carbon (10%) were added and the mixture was hydrogenated with hydrogen at RT and standard pressure for 1 h. The catalyst was filtered off and the reaction mixture was freed from the solvent under reduced pressure and dried under high vacuum. The residue was purified by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 29.4 mg (50% of theory) of the title compound.

LC-MS (Method 5): Rₜ=3.77 min; MS (ESIpos): m/z=1021 [M+H]⁺

### Intermediate C81

### (R)-1-[1-Benzyl-4-(2,5-difluorophenyl)-1H-pyrrol-2-yl]-1-cyclohexylmethanamine

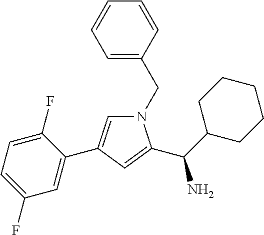 [SMILES: N[C@@H](c1cc(-c2cc(F)ccc2F)cn1Cc1ccccc1)C1CCCCC1]

Under argon and at −78° C., 18.7 ml (37.45 mmol) of cyclohexylmagnesium chloride in diethyl ether (2M) were added to a solution of 3.12 ml (6.24 mmol) of dimethylzinc in toluene (2.0 M), and the mixture was stirred at −78° C. for 30 minutes. A solution of 5.0 g (12.48 mmol) of (R)—N-{(E/Z)-[1-benzyl-4-(2,5-difluorophenyl)-1H-pyrrol-2-yl]methylene}-2-methylpropane-2-sulphinamide in THF was then added at −78° C., and the reaction mixture was stirred at this temperature for 1 h and then at RT for 4 h. At −78° C., saturated ammonium chloride solution was then added and the reaction mixture was allowed to warm to RT. The mixture was diluted with ethyl acetate and washed with water. The organic phase was dried over magnesium sulphate and the solvent was evaporated under reduced pressure. The residue was purified using Biotage Isolera (silica gel, ethyl acetate/cyclohexane 25:75). This gave 1.59 g (26% of theory) of the intermediate.

LC-MS (Method 12): Rₜ=2.76 min; MS (ESIneg): m/z=483 [M−H]⁻

Under argon, 264.0 mg (0.54 mmol) of this intermediate were initially charged in 0.5 ml of 1,4-dioxane, and 1.36 ml of HCl in 1,4-dioxane solution (4.0 M) were then added. The reaction mixture was stirred at RT for 1 h. Dichloromethane was added, and the reaction mixture was washed with an aqueous 1M sodium hydroxide solution. The organic phase was dried with magnesium sulphate and the solvent was evaporated under reduced pressure. The residue was purified using Biotage Isolera (silica gel, methanol/dichloromethane 98:2). The solvent was evaporated under reduced pressure and the residue was dissolved in dichloromethane, washed with a sodium bicarbonate solution and dried over sodium sulphate. The solvent was evaporated under reduced pressure and the residue was dried under high vacuum. This gave 148 mg (72% of theory) of the title compound.

LC-MS (Method 13): Rₜ=2.07 min; MS (ESIpos): m/z=364 [M-NH₂]⁺

### Intermediate C82

### 2-(Trimethylsilyl)ethyl (3-{[(R)-[1-benzyl-4-(2,5-difluorophenyl)-1H-pyrrol-2-yl](cyclohexyl)methyl]amino}propyl)carbamate

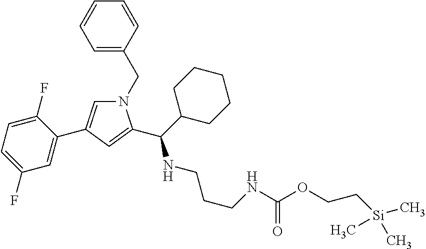 [SMILES: C[Si](C)(C)CCOC(=O)NCCCN[C@@H](c1cc(-c2cc(F)ccc2F)cn1Cc1ccccc1)C1CCCCC1]

Under argon, 392.2 mg (1.85 mmol) of sodium triacetoxyborohydride and 91.29 mg (1.52 mmol) of acetic acid were added to a solution of 503.0 mg (1.32 mmol) of 1-[1-benzyl-4-(2,5-difluorophenyl)-1H-pyrrol-2-yl]-1-cyclohexylmethanamine (Intermediate C81) in 1.4 ml of dichloromethane, and the reaction mixture was stirred at RT for 10 minutes. A solution of 574.6 (2.38 mmol) of 2-(trimethylsilyl)ethyl (3-oxopropyl)carbamate in dichloromethane was then added, and the mixture was stirred at RT overnight. After addition of 143 mg (0.66 mmol) of 2-(trimethylsilyl)ethyl (3-oxopropyl)carbamate, the mixture was stirred for a further 2 h. The reaction mixture was diluted with dichloromethane and the organic phase was washed in each case twice with saturated sodium carbonate solution and with saturated NaCl solution, dried over sodium sulphate and concentrated. The residue was purified by preparative HPLC. The solvents were evaporated under reduced pressure and the residue was dried under high vacuum. This gave 488 g (63% of theory) of the title compound.

LC-MS (Method 12): Rₜ=1.89 min; MS (ESIpos): m/z=582 (M+H)⁺.

### Intermediate C83

### 2-(Trimethylsilyl)ethyl (3-{[(R)-[1-benzyl-4-(2,5-difluorophenyl)-1H-pyrrol-2-yl](cyclohexyl)methyl](chloroacetyl)amino}propyl)carbamate

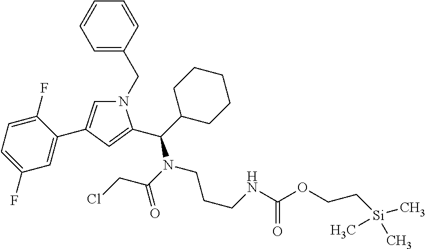 [SMILES: C[Si](C)(C)CCOC(=O)NCCCN(C(=O)CCl)[C@@H](c1cc(-c2cc(F)ccc2F)cn1Cc1ccccc1)C1CCCCC1]

280.0 mg (2.77 mmol) of triethylamine and 397.8 mg (3.52 mmol) of chloroacetyl chloride were added to a solution of 487.9 mg (0.84 mmol) 2-(trimethylsilyl)ethyl (3-{[(R)-[1-benzyl-4-(2,5-difluorophenyl)-1H-pyrrol-2-yl](cyclohexyl)methyl]amino}propyl)carbamate (Intermediate C82) in 8.40 ml of dichloromethane with 4 Å molecular sieve, and the reaction mixture was stirred at RT for 6 h. The reaction mixture was diluted with dichloromethane and the organic phase was washed with saturated sodium bicarbonate solution and saturated ammonium chloride solution. The organic phase was dried over sodium sulphate and concentrated. The residue was used further without purification. This gave 470 mg (85% of theory) of the title compound.

LC-MS (Method 12): Rₜ=2.88 min; MS (ESIpos): m/z=680 (M+Na)⁺.

### Intermediate C84

### S-{11-[(R)-[1-Benzyl-4-(2,5-difluorophenyl)-1H-pyrrol-2-yl](cyclohexyl)methyl]-2,2-dimethyl-6,12-dioxo-5-oxa-7,11-diaza-2-silatridecan-13-yl}-L-cysteine

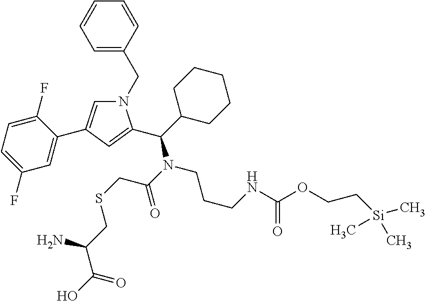 [SMILES: C[Si](C)(C)CCOC(=O)NCCCN(C(=O)CSC[C@H](N)C(=O)O)[C@@H](c1cc(-c2cc(F)ccc2F)cn1Cc1ccccc1)C1CCCCC1]

322.1 mg (2.66 mmol) of L-cysteine were suspended in 0.19 ml of water together with 319.0 mg (3.80 mmol) of sodium bicarbonate. 250.0 mg (0.38 mmol) of 2-(trimethylsilyl)ethyl (3-{[(R)-[1-benzyl-4-(2,5-difluorophenyl)-1H-pyrrol-2-yl](cyclohexyl)methyl](chloroacetyl)amino}propyl)carbamate (Intermediate C83) dissolved in 1.90 ml of isopropanol and 693.8 g (4.56 mmol) of 1,8-diazabicyclo[5.4.0]undec-7-ene were added. The reaction mixture was stirred at 50° C. for 3.5 h. Ethyl acetate was added to the reaction mixture and the organic phase was washed repeatedly with saturated sodium bicarbonate solution and once with saturated NaCl solution. The organic phase was dried over sodium sulphate and the solvent was evaporated under reduced pressure. The residue was used further without further purification. This gave 276 mg (97% of theory) of the title compound.

LC-MS (Method 12): Rₜ=2.34 min; MS (ESIpos): m/z=744 (M+H)⁺.

### Intermediate C85

### S-{11-[(R)-[1-Benzyl-4-(2,5-difluorophenyl)-1H-pyrrol-2-yl](cyclohexyl)methyl]-2,2-dimethyl-6,12-dioxo-5-oxa-7,11-diaza-2-silatridecan-13-yl}-N-[6-(2,5-dioxo-2,5-dihydro-1H-pyrrol-1-yl)hexanoyl]-L-cysteine

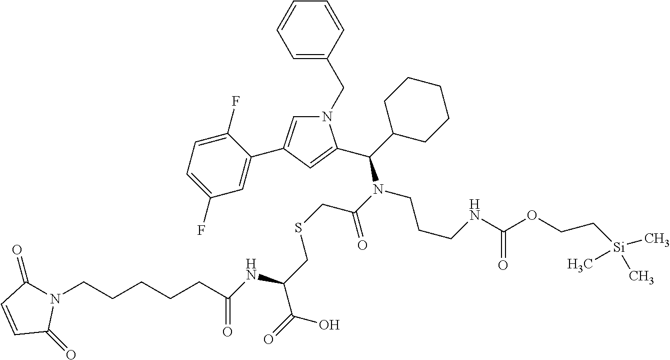 [SMILES: C[Si](C)(C)CCOC(=O)NCCCN(C(=O)CSC[C@H](NC(=O)CCCCCN1C(=O)C=CC1=O)C(=O)O)[C@@H](c1cc(-c2cc(F)ccc2F)cn1Cc1ccccc1)C1CCCCC1]

34.8 mg (0.27 mmol) of N,N-diisopropylethylamine were added to a mixture of 100 mg (0.13 mmol) of S-{11-[(R)-[1-benzyl-4-(2,5-difluorophenyl)-1H-pyrrol-2-yl](cyclohexyl)methyl]-2,2-dimethyl-6,12-dioxo-5-oxa-7,11-diaza-2-silatridecan-13-yl}-L-cysteine (1:1) (Intermediate C84) and 41.5 mg (0.13 mmol) of 1-{6-[(2,5-dioxopyrrolidin-1-yl)oxy]-6-oxohexyl}-1H-pyrrole-2,5-dione in 4.0 ml of DMF, and the reaction mixture was stirred at RT for 3 h. Without work-up, the mixture was purified by preparative HPLC. This gave 88 mg (70% of theory) of the title compound.

LC-MS (Method 12): Rₜ=2.71 min; MS (ESIpos): m/z=936 (M+H)⁺.

### Intermediate C86

### 11-[(R)-[1-Benzyl-4-(2,5-difluorophenyl)-1H-pyrrol-2-yl](cyclohexyl)methyl]-2,2-dimethyl-6,12-dioxo-5-oxa-14-thia-7,11-diaza-2-silaheptadecan-17-oic acid

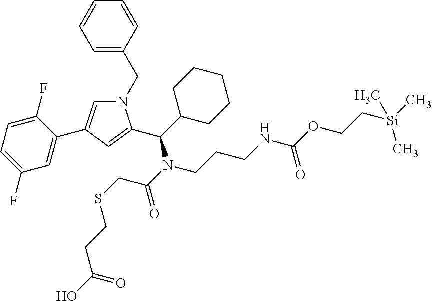 [SMILES: C[Si](C)(C)CCOC(=O)NCCCN(C(=O)CSCCC(=O)O)[C@@H](c1cc(-c2cc(F)ccc2F)cn1Cc1ccccc1)C1CCCCC1]

161.65 mg (1.17 mmol) of potassium carbonate were added to a mixture of 220.0 mg (0.33 mmol) of 2-(trimethylsilyl)ethyl (3-{[(R)-[1-benzyl-4-(2,5-difluorophenyl)-1H-pyrrol-2-yl](cyclohexyl)methyl](chloroacetyl)amino}propyl)carbamate (Intermediate C83) and 39.02 mg (0.37 mmol) of 3-sulphanylpropanoic acid in 7.45 ml of methanol and a few drops of water. The reaction mixture was stirred at 50° C. for 4 h. Ethyl acetate was added to the reaction mixture and the organic phase was washed repeatedly with water and with saturated NaCl solution. The organic phase was dried over sodium sulphate and the solvent was evaporated under reduced pressure. The residue was used further without work-up. This gave 201 mg (83% of theory) of the title compound.

LC-MS (Method 12): Rₜ=2.72 min; MS (ESIneg): m/z=726 (M−H)⁻.

### Intermediate C87

### 2-(Trimethylsilyl)ethyl {13-[(R)-[1-benzyl-4-(2,5-difluorophenyl)-1H-pyrrol-2-yl](cyclohexyl)methyl]-1-(2,5-dioxo-2,5-dihydro-1H-pyrrol-1-yl)-2,7,12-trioxo-10-thia-3,6,13-diazahexadecan-16-yl}carbamate

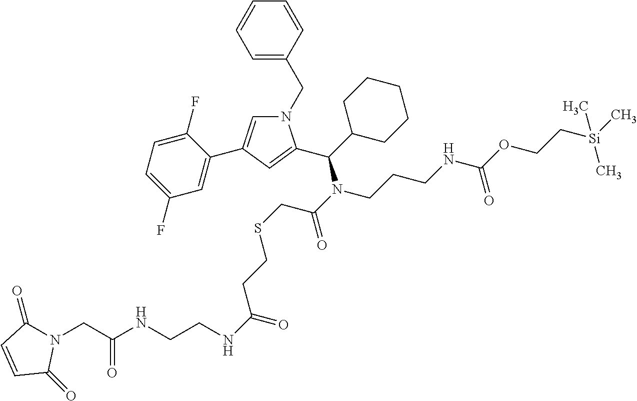 [SMILES: C[Si](C)(C)CCOC(=O)NCCCN(C(=O)CSCCC(=O)NCCNC(=O)CN1C(=O)C=CC1=O)[C@@H](c1cc(-c2cc(F)ccc2F)cn1Cc1ccccc1)C1CCCCC1]

54.18 mg (0.28 mmol) of N-(2-aminoethyl)-2-(2,5-dioxo-2,5-dihydro-1H-pyrrol-1-yl)acetamide (Intermediate L1), 71.01 mg (0.50 mmol) of N,N-diisopropylethylamine, 104.46 mg (0.27 mmol) of HATU and 0.23 ml (0.14 mmol) of 1-hydoxy-7-azabenzotriazole 0.5 M in DMF were added to a solution of 100 mg (0.14 mmol) of 11-[(R)-[1-benzyl-4-(2,5-difluorophenyl)-1H-pyrrol-2-yl](cyclohexyl)methyl]-2,2-dimethyl-6,12-dioxo-5-oxa-14-thia-7,11-diaza-2-silaheptadecan-17-oic acid (Intermediate C86) in 1.37 ml of DMF. The reaction mixture was stirred at RT for 5 h. Without further work-up, the mixture was purified by preparative HPLC. This gave 41 mg (33% of theory) of the title compound.

LC-MS (Method 12): Rₜ=2.61 min; MS (ESIpos): m/z=907 (M+H)⁺.

### Intermediate C88

tert-Butyl 3-[({(1R)-1-[1-benzyl-4-(2,5-difluorophenyl)-1H-pyrrol-2-yl]-2,2-dimethylpropyl}amino)methyl]pyrrolidine-1-carboxylate trifluoroacetic acid (1:1)

### Mixture of Stereoisomers

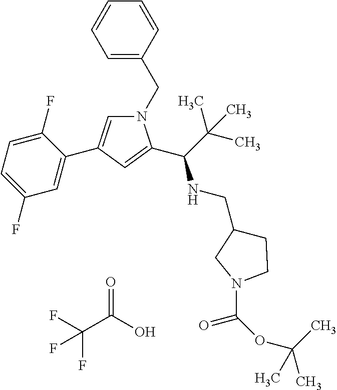 [SMILES: CC(C)(C)OC(=O)N1CCC(CN[C@@H](c2cc(-c3cc(F)ccc3F)cn2Cc2ccccc2)C(C)(C)C)C1 ; O=C(O)C(F)(F)F]

1.71 g (8.05 mmol) of sodium triacetoxyborohydride and 0.40 g (6.61 mmol) of acetic acid were added to a solution of 2.04 mg (5.75 mmol) of (1R)-1-[1-benzyl-4-(2,5-difluorophenyl)-1H-pyrrol-2-yl]-2,2-dimethylpropane-1-amine (Intermediate C52) in 51 ml of dichloromethane, and the reaction mixture was stirred at RT for 5 minutes. A solution of 1.32 g (6.61 mmol) of tert-butyl 3-formylpyrrolidine-1-carboxylate in 20 ml of dichloromethane was then added, and the mixture was stirred at RT overnight. The reaction mixture was diluted with ethyl acetate and the organic phase was washed in each case twice with saturated sodium carbonate solution and with saturated NaCl solution, dried over magnesium sulphate and concentrated. The residue was purified by preparative HPLC. The solvents were evaporated under reduced pressure and the residue was dried under high vacuum. This gave 1.86 g (50% of theory) of the title compound.

LC-MS (Method 1): Rₜ=0.99 min; MS (ESIpos): m/z=538 (M+H—CF₃CO₂H)⁺.

### Intermediate C89

### tert-Butyl 3-{[{(1R)-1-[1-benzyl-4-(2,5-difluorophenyl)-1H-pyrrol-2-yl]-2,2-dimethylpropyl}(chloroacetyl)amino]methyl}pyrrolidine-1-carboxylate

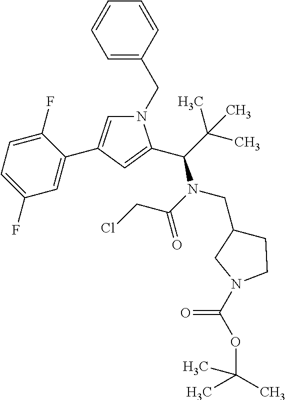 [SMILES: CC(C)(C)OC(=O)N1CCC(CN(C(=O)CCl)[C@@H](c2cc(-c3cc(F)ccc3F)cn2Cc2ccccc2)C(C)(C)C)C1]

1.36 g (13.42 mmol) of triethylamine and 2.13 g (18.87 mmol) of chloracetyl chloride were added to a solution of 2.89 g (4.19 mmol, 80% pure) of tert-butyl 3-[({(1R)-1-[1-benzyl-4-(2,5-difluorophenyl)-1H-pyrrol-2-yl]-2,2-dimethylpropyl}amino)methyl]pyrrolidine-1-carboxylate (Intermediate C88) in 42 ml of dichloromethane with 4 Å molecular sieve. The reaction mixture was stirred at RT for 5 h. The mixture was concentrated on a rotary evaporator and the residue was purified by preparative HPLC. This gave 449 mg (17% of theory) of Isomer 1 and 442 mg (17% of theory) of Isomer 2 of the title compound.

Isomer 1 LC-MS (Method 1): Rₜ=2.74 min; MS (ESIpos): m/z=614 (M+H)⁺.

Isomer 2 LC-MS (Method 1): Rₜ=2.78 min; MS (ESIpos): m/z=614 (M+H)⁺.

### Intermediate C90

### S-[2-({(1R)-1-[1-Benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]-L-cysteine (Isomer 1)

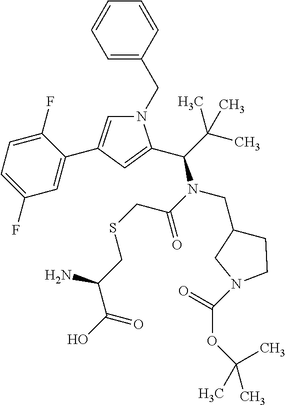 [SMILES: CC(C)(C)OC(=O)N1CCC(CN(C(=O)CSC[C@H](N)C(=O)O)[C@@H](c2cc(-c3cc(F)ccc3F)cn2Cc2ccccc2)C(C)(C)C)C1]

357.3 mg (0.58 mmol) of L-cysteine were suspended in 2.3 ml of water together with 488.7 mg (4.07 mmol) of sodium bicarbonate. 357.0 mg (0.58 mmol) of tert-butyl 3-{[{(1R)-1-[1-benzyl-4-(2,5-difluorophenyl)-1H-pyrrol-2-yl]-2,2-dimethylpropyl}(chloroacetyl)amino]methyl}pyrrolidine-1-carboxylate (Isomer 1)

(Intermediate C89, Isomer 1) dissolved in 23.0 ml of isopropanol and 1.06 g (6.98 mmol) of 1,8-diazabicyclo[5.4.0]undec-7-ene were added. The reaction mixture was stirred at 50° C. for 3 h. Ethyl acetate was added to the reaction mixture and the organic phase was washed repeatedly with saturated sodium bicarbonate solution and once with sat. NaCl solution. The organic phase was dried over magnesium sulphate and the solvent was evaporated under reduced pressure. The residue was used further without purification. This gave 255.0 mg (62% of theory) of the title compound.

LC-MS (Method 1): Rₜ=1.09 min; MS (ESIpos): m/z=699 (M+H)⁺.

### Intermediate C91

### S-[2-({(1R)-1-[1-Benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]-L-cysteine (Isomer 2)

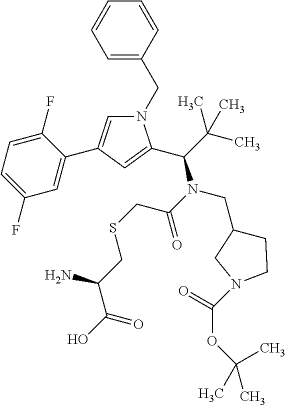 [SMILES: CC(C)(C)OC(=O)N1CCC(CN(C(=O)CSC[C@H](N)C(=O)O)[C@@H](c2cc(-c3cc(F)ccc3F)cn2Cc2ccccc2)C(C)(C)C)C1]

453.5 mg (3.74 mmol) of L-cysteine were suspended in 2.1 ml of water together with 449.2 mg (5.35 mmol) of sodium bicarbonate. 3287.4 mg (0.54 mmol) of tert-butyl 3-{[{(1R)-1-[1-benzyl-4-(2,5-difluorophenyl)-1H-pyrrol-2-yl]-2,2-dimethylpropyl}(chloroacetyl)amino]methyl}pyrrolidine-1-carboxylate (Intermediate C89, Isomer 2) dissolved in 21.1 ml of isopropanol and 0.98 g (6.42 mmol) of 1,8-diazabicyclo[5.4.0]undec-7-ene were added. The reaction mixture was stirred at 50° C. for 3 h. Ethyl acetate was added to the reaction mixture and the organic phase was washed repeatedly with saturated sodium bicarbonate solution and once with sat. NaCl solution. The organic phase was dried over magnesium sulphate and the solvent was evaporated under reduced pressure. The residue was used further without purification. This gave 221.0 mg (59% of theory) of the title compound.

LC-MS (Method 1): Rₜ=1.12 min; MS (ESIpos): m/z=699 (M+H)⁺.

### Intermediate C92

### S-[2-({(1R)-1-[1-Benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]-N-[6-(2,5-dioxo-2,5-dihydro-1H-pyrrol-1-yl)hexanoyl]-L-cysteine (Isomer 1)

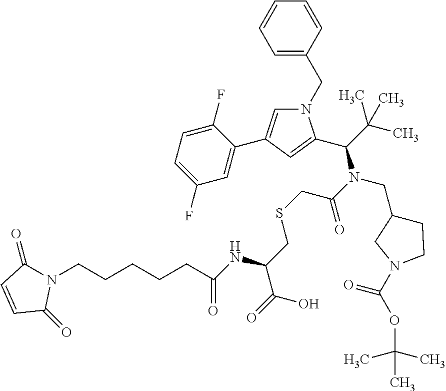 [SMILES: CC(C)(C)OC(=O)N1CCC(CN(C(=O)CSC[C@H](NC(=O)CCCCCN2C(=O)C=CC2=O)C(=O)O)[C@@H](c2cc(-c3cc(F)ccc3F)cn2Cc2ccccc2)C(C)(C)C)C1]

18.49 mg (0.14 mmol) of N,N-diisopropylethylamine were added to a mixture of 50 mg (0.07 mmol) of S-[2-({(1R)-1-[1-benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]-L-cysteine (Intermediate C90) and 22.06 mg (0.07 mmol) of 1-{6-[(2,5-dioxopyrrolidin-1-yl)oxy]-6-oxohexyl}-1H-pyrrole-2,5-dione in 3.3 ml of DMF, and the reaction mixture was stirred at RT for 45 minutes. Without work-up, the mixture was purified by preparative HPLC. This gave 65 mg (100% of theory, 71% pure) of the title compound.

LC-MS (Method 1): Rₜ=1.31 min; MS (ESIpos): m/z=892 (M+H)⁺.

### Intermediate C93

### S-[2-({(1R)-1-[1-Benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]-N-[6-(2,5-dioxo-2,5-dihydro-1H-pyrrol-1-yl)hexanoyl]-L-cysteine (Isomer 2)

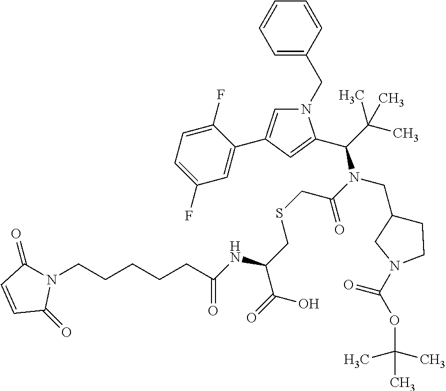 [SMILES: CC(C)(C)OC(=O)N1CCC(CN(C(=O)CSC[C@H](NC(=O)CCCCCN2C(=O)C=CC2=O)C(=O)O)[C@@H](c2cc(-c3cc(F)ccc3F)cn2Cc2ccccc2)C(C)(C)C)C1]

18.49 mg (0.14 mmol) of N,N-diisopropylethylamine were added to a mixture of 50.0 mg (0.07 mmol) of S-[2-({(1R)-1-[1-benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]-L-cysteine (Intermediate C91) and 22.06 mg (0.07 mmol) of 1-{6-[(2,5-dioxopyrrolidin-1-yl)oxy]-6-oxohexyl}-1H-pyrrole-2,5-dione in 3.0 ml of DMF, and the reaction mixture was stirred at RT for 90 minutes. Without work-up, the mixture was purified by preparative HPLC. This gave 63 mg (98% of theory, 73% pure) of the title compound.

LC-MS (Method 1): Rₜ=1.34 min; MS (ESIpos): m/z=892 (M+H)⁺.

### Intermediate C94

### S-[2-({(1R)-1-[1-Benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]-N-[(2,5-dioxo-2,5-dihydro-1H-pyrrol-1-yl)acetyl]-L-cysteine (Isomer 1)

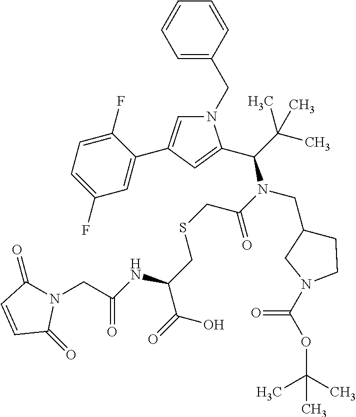 [SMILES: CC(C)(C)OC(=O)N1CCC(CN(C(=O)CSC[C@H](NC(=O)CN2C(=O)C=CC2=O)C(=O)O)[C@@H](c2cc(-c3cc(F)ccc3F)cn2Cc2ccccc2)C(C)(C)C)C1]

18.5 mg (0.14 mmol) of N,N-diisopropylethylamine were added to a mixture of 50.0 mg (0.07 mmol) of S-[2-({(1R)-1-[1-benzyl-4-(2,5-difluorophenyl)-1H-pyrrol-2-yl]-2,2-dimethylpropyl}{[-1-(tert-butoxycarbonyl)pyrrolidin-3-yl]methyl}amino)-2-oxoethyl]-L-cysteine (Intermediate C90) and 18.0 mg (0.07 mmol) of 1-{2-[(2,5-dioxopyrrolidin-1-yl)oxy]-2-oxoethyl}-1H-pyrrole-2,5-dione in 3.3 ml of DMF, and the reaction mixture was stirred at RT for 30 minutes. Ethyl acetate was added to the reaction mixture and the organic phase was washed repeatedly with saturated NH₄Cl solution and once with saturated NaCl solution. The organic phase was dried over magnesium sulphate and the solvent was evaporated under reduced pressure. The residue was employed without further purification. This gave 57 mg (81% of theory, 85% pure) of the title compound.

LC-MS (Method 1): Rₜ=0.96 min; MS (ESIpos): m/z=836 (M+H)⁺.

### Intermediate C95

### 3-{[2-({(1R)-1-[1-Benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]sulphanyl}propanoic acid (Isomer 1)

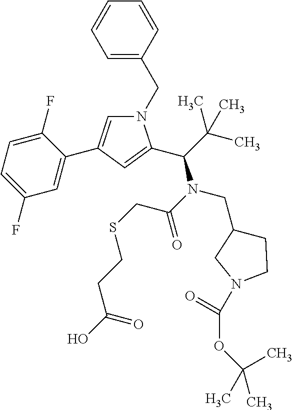 [SMILES: CC(C)(C)OC(=O)N1CCC(CN(C(=O)CSCCC(=O)O)[C@@H](c2cc(-c3cc(F)ccc3F)cn2Cc2ccccc2)C(C)(C)C)C1]

302.5 mg (2.19 mmol) of potassium carbonate were added to a mixture of 384.0 mg (0.62 mmol) of tert-butyl 3-{[{(1R)-1-[1-benzyl-4-(2,5-difluorophenyl)-1H-pyrrol-2-yl]-2,2-dimethylpropyl}(chloroacetyl)amino]methyl}pyrrolidine-1-carboxylate (Intermediate C89, Isomer 1) and 73.0 mg (0.69 mmol) of 3-sulphanylpropanoic acid in 14 ml of methanol and a few drops of water. The reaction mixture was stirred at 50° C. for 2.5 h. Ethyl acetate was added to the reaction mixture and the organic phase was washed repeatedly with water and with saturated NaCl solution. The organic phase was dried over magnesium sulphate, the solvent was evaporated under reduced pressure and the residue was dried under high vacuum. The residue was used further without work-up. This gave 358.0 mg (84% of theory) of the title compound.

LC-MS (Method 1): Rₜ=1.33 min; MS (ESIpos): m/z=684 (M+H)⁺.

### Intermediate C96

### 3-{[2-({(1R)-1-[1-Benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]sulphanyl}propanoic acid (Isomer 2)

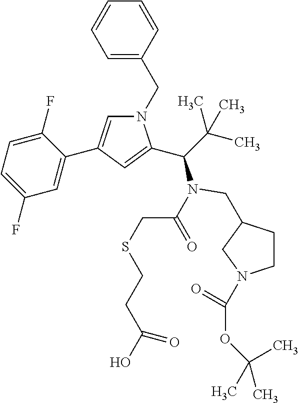 [SMILES: CC(C)(C)OC(=O)N1CCC(CN(C(=O)CSCCC(=O)O)[C@@H](c2cc(-c3cc(F)ccc3F)cn2Cc2ccccc2)C(C)(C)C)C1]

226.0 mg (1.64 mmol) of potassium carbonate were added to a mixture of 287.0 mg (0.45 mmol) of tert-butyl 3-{[{(1R)-1-[1-benzyl-4-(2,5-difluorophenyl)-1H-pyrrol-2-yl]-2,2-dimethylpropyl}(chloroacetyl)amino]methyl}pyrrolidine-1-carboxylate (Intermediate C89, Isomer 2) and 54.6 mg (0.51 mmol) of 3-sulphanylpropanoic acid in 14 ml of methanol and a few drops of water. The reaction mixture was stirred at 50° C. for 2.5 h. Ethyl acetate was added to the reaction mixture and the organic phase was washed repeatedly with water and with saturated NaCl solution. The organic phase was dried over magnesium sulphate, the solvent was evaporated under reduced pressure and the residue was dried under high vacuum. The residue was used further without work-up. This gave 318.7 mg (88% of theory, 88% pure) of the title compound.

LC-MS (Method 1): Rₜ=1.36 min; MS (ESIpos): m/z=684 (M+H)⁺.

### Intermediate C97

### tert-Butyl 3-[2-{(1R)-1-[1-benzyl-4-(2,5-difluorophenyl)-1H-pyrrol-2-yl]-2,2-dimethylpropyl}-14-(2,5-dioxo-2,5-dihydro-1H-pyrrol-1-yl)-3,8,13-trioxo-5-thia-2,9,12-triazatetradec-1-yl]pyrrolidine-1-carboxylate (Isomer 2)

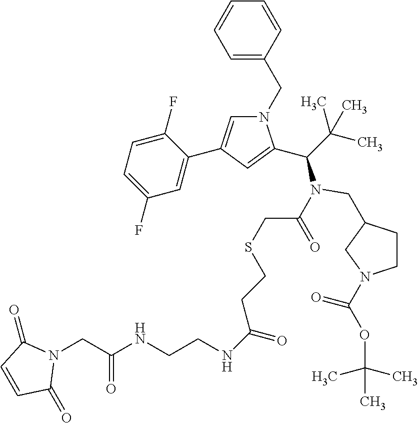 [SMILES: CC(C)(C)OC(=O)N1CCC(CN(C(=O)CSCCC(=O)NCCNC(=O)CN2C(=O)C=CC2=O)[C@@H](c2cc(-c3cc(F)ccc3F)cn2Cc2ccccc2)C(C)(C)C)C1]

Under argon, 14.17 mg (0.11 mmol) of N,N-diisopropylethylamin and 27.80 mg (0.07 mmol) of HATU were added to a solution of 25.0 mg (0.04 mmol) of 3-{[2-({(1R)-1-[1-benzyl-4-(2,5-difluorophenyl)-1H-pyrrol-2-yl]-2,2-dimethylpropyl}{[1-(tert-butoxycarbonyl)pyrrolidin-3-yl]-methyl}amino)-2-oxoethyl]sulphanyl}propanoic acid (Intermediate C96) in 2.81 ml of DMF. The reaction mixture was stirred at RT for 10 minutes. A solution of 22.75 mg (0.07 mmol) of N-(2-aminoethyl)-2-(2,5-dioxo-2,5-dihydro-1H-pyrrol-1-yl)acetamide-ethane (1:1) trifluoroacetic acid (Intermediate L1) in 1.4 ml of DMF and 5 mg (0.04 mmol) of N,N-diisopropylethylamine was then added, and the mixture was stirred at RT overnight. Water was added and the mixture was extracted with dichloromethane. The organic phase was dried over magnesium sulphate and the solvent was evaporated under reduced pressure. The residue was used further without work-up. This gave 26 mg (84% of theory) of the title compound.

LC-MS (Method 5): Rₜ=4.39 min; MS (ESIpos): m/z=863 (M+H)⁺.

### Intermediate C98

### tert-Butyl 3-[2-{(1R)-1-[1-benzyl-4-(2,5-difluorophenyl)-1H-pyrrol-2-yl]-2,2-dimethylpropyl}-18-(2,5-dioxo-2,5-dihydro-1H-pyrrol-1-yl)-3,8,13-trioxo-5-thia-2,9,12-triazaoctadec-1-yl]pyrrolidine-1-carboxylate (Isomer 2)

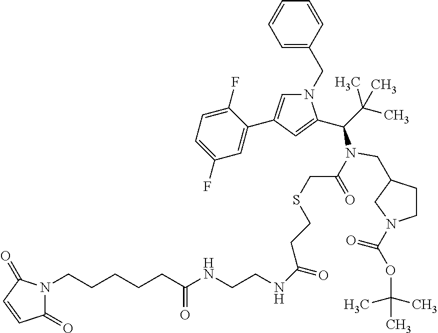 [SMILES: CC(C)(C)OC(=O)N1CCC(CN(C(=O)CSCCC(=O)NCCNC(=O)CCCCCN2C(=O)C=CC2=O)[C@@H](c2cc(-c3cc(F)ccc3F)cn2Cc2ccccc2)C(C)(C)C)C1]

Under argon, 14.17 mg (0.11 mmol) of N,N-diisopropylethylamine and 27.80 mg (0.07 mmol) of HATU were added to a solution of 25.0 mg (0.04 mmol) of 3-{[2-({(1R)-1-[1-benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]sulphanyl}propanoic acid (Intermediate C96) in 2.81 ml of DMF. The reaction mixture was stirred at RT for 10 minutes. A solution of 37.30 mg (0.07 mmol) of N-(2-aminoethyl)-6-(2,5-dioxo-2,5-dihydro-1H-pyrrol-1-yl)hexanamide-ethane (1:1) trifluoroacetic acid in 1.4 ml of DMF and 5 mg (0.04 mmol) of N,N-diisopropylethylamine was then added, and the mixture was stirred at RT overnight. Water was added and the mixture was extracted with dichloromethane. The organic phase was dried over magnesium sulphate and the solvent was evaporated under reduced pressure. The residue was employed without further purification. This gave 22 mg (63% of theory) of the title compound.

LC-MS (Method 5): Rₜ=4.54 min; MS (ESIpos): m/z=919 (M+H)⁺.

### Intermediate C99

### tert-Butyl 3-[2-{(1R)-1-[1-benzyl-4-(2,5-difluorophenyl)-1H-pyrrol-2-yl]-2,2-dimethylpropyl}-24-(2,5-dioxo-2,5-dihydro-1H-pyrrol-1-yl)-3,8,19-trioxo-12,15-dioxa-5-thia-2,9,18-triazatetracos-1-yl]pyrrolidine-1-carboxylate (Isomer 2)

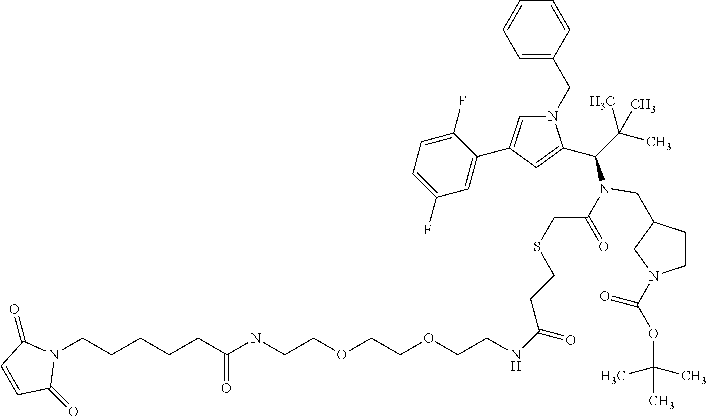 [SMILES: CC(C)(C)OC(=O)N1CCC(CN(C(=O)CSCCC(=O)NCCOCCOCCNC(=O)CCCCCN2C(=O)C=CC2=O)[C@@H](c2cc(-c3cc(F)ccc3F)cn2Cc2ccccc2)C(C)(C)C)C1]

Under argon, 14.17 mg (0.11 mmol) of N,N-diisopropylethylamine and 27.80 mg (0.07 mmol) of HATU were added to a solution of 25.0 mg (0.04 mmol) of 3-{[2-({(1R)-1-[1-benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]sulphanyl}propanoic acid (Intermediate C96) in 2.81 ml of DMF. The reaction mixture was stirred at RT for 10 minutes. A solution of 35.05 mg (0.07 mmol) of N-{2-[2-(2-Aminoethoxy)ethoxy]ethyl}-6-(2,5-dioxo-2,5-dihydro-1H-pyrrol-1-yl)hexan amide-ethane (1:1) trifluoroacetic acid (Intermediate L82) in 1.4 ml of DMF and 5 mg (0.04 mmol) of N,N-diisopropylethylamine was then added, and the mixture was stirred at RT overnight. Water was added and the mixture was extracted with dichloromethane. The organic phase was dried over magnesium sulphate, the solvent was evaporated under reduced pressure and the residue was dried under high vacuum. The residue was purified by prep. HPLC. This gave 25 mg (60% of theory) of the title compound.

LC-MS (Method 1): Rₜ=4.52 min; MS (ESIpos): m/z=1007 (M+H)⁺.

### Intermediate C100

### 2-(Trimethylsilyl)ethyl {(2S)-4-[{(1R)-1-[1-benzyl-4-(2,5-difluorophenyl)-1H-pyrrol-2-yl]-2,2-dimethylpropyl}(glycoloyl)amino]-1-[(2-{[(2R)-2-(2,5-dioxo-2,5-dihydro-1H-pyrrol-1-yl)propanoyl]amino}ethylamino]-1-oxobutan-2-yl}carbamate

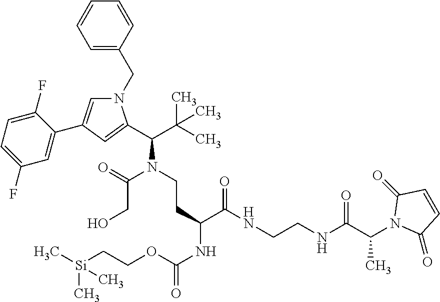 [SMILES: C[C@H](C(=O)NCCNC(=O)[C@H](CCN(C(=O)CO)[C@@H](c1cc(-c2cc(F)ccc2F)cn1Cc1ccccc1)C(C)(C)C)NC(=O)OCC[Si](C)(C)C)N1C(=O)C=CC1=O]

22.2 mg (0.068 mmol) of (2R)—N-(2-aminoethyl)-2-(2,5-dioxo-2,5-dihydro-1H-pyrrol-1-yl)propanamide (1:1) trifluoroacetic acid were added to a solution of 45 mg (0.068 mmol) of (2S)-4-[{(1R)-1-[1-benzyl-4-(2,5-difluorophenyl)-1H-pyrrol-2-yl]-2,2-dimethylpropyl}(glycoloyl)amino]-2-({[2-(trimethylsilyl)ethoxy]carbonyl}aminobutanoic acid (Intermediate C58) in 5.8 ml of DMF. After 30 minutes of stirring at RT, 39 mg (0.10 mmol) of HATU and 36 mg (0.27 mmol) of N,N-diisopropylethylamine were added to the mixture. The reaction mixture was stirred at RT for 1 h. Without work-up, the mixture was purified by preparative HPLC. This gave 7 mg (12% of theory) of the title compound.

LC-MS (Method 1): Rₜ=1.41 min; MS (ESIpos): m/z 851 (M+H)⁺.

### Intermediate C101

### Trifluoroacetic acid/methyl (2S)-4-[(acetoxyacetyl) {(1R)-1-[1-benzyl-4-(2,5-difluorophenyl)-1H-pyrrol-2-yl]-2,2-dimethylpropyl}amino]-2-aminobutanoate (1:1)

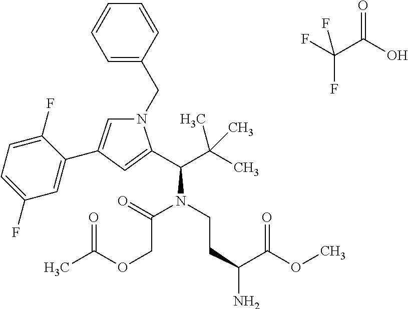 [SMILES: COC(=O)[C@@H](N)CCN(C(=O)COC(C)=O)[C@@H](c1cc(-c2cc(F)ccc2F)cn1Cc1ccccc1)C(C)(C)C ; O=C(O)C(F)(F)F]

4.3 g (12.2 mmol) of intermediate C52 were dissolved in 525 ml of DCM, and 3.63 g (17.12 mmol) of sodium triacetoxyborohydride and 8.4 ml of acetic acid were added. After 5 min of stirring at RT, 3.23 g (11.85 mmol) of methyl (2S)-4-oxo-2-({[2-(trimethylsilyl)ethoxy]carbonyl}amino)butanoate (prepared from (3S)-3-amino-4-methoxy-4-oxobutanoic acid using classical methods), dissolved in 175 ml of DCM, were added and the mixture was stirred at RT for a further 45 min. The mixture was then diluted with DCM and extracted twice with 100 ml of saturated sodium bicarbonate solution and then with saturated sodium chloride solution. The organic phase was dried over magnesium sulphate, filtered and then concentrated. The residue was purified by preparative HPLC. Combining the appropriate fractions, concentration and drying of the residue under high vacuum gave 4.6 g (61% of theory) of the intermediate.

LC-MS (Method 12): Rₜ=1.97 min; MS (ESIpos): m/z=614.32 (M+H)⁺.

2.06 g (3.36 mmol) of this intermediate were initially charged in 76 ml of DCM and acylated using 0.81 ml (7.17 mmol) of 2-chloro-2-oxoethyl acetate in the presence of 2.1 ml of triethylamine. After 20 h of stirring at RT, a further 0.36 ml of 2-chloro-2-oxoethyl acetate and 0.94 ml of triethylamine were added, and the mixture was stirred at RT for a further 15 min. The mixture was then diluted with 500 ml of ethyl acetate and extracted successively twice with 300 ml of 5% strength citric acid, twice with 300 ml of saturated sodium bicarbonate solution and once with 100 ml of saturated sodium chloride solution and then dried over magnesium sulphate and concentrated. Drying under high vacuum gave 2.17 g (79% of theory) of the protected intermediate.

LC-MS (Method 1): Rₜ=1.48 min; MS (ESIpos): m/z=714 (M+H)⁺.

321 mg (0.342 mmol) of this intermediate were dissolved in 7 ml of 2,2,2-trifluoroethanol. 279.5 mg (2.05 mmol) of zinc chloride were added, and the mixture was stirred at 50° C. for 2 h. 599 mg (2.05 mmol) of ethylenediamine-N,N,N′,N′-tetraacetic acid and 2 ml of a 0.1% strength solution of trifluoroacetic acid in water were then added, and the mixture was subsequently concentrated under reduced pressure. The residue was purified by preparative HPLC. Concentration of the appropriate fractions and lyophilization of the residue from acetonitrile/water gave 60 mg (26% of theory) of the title compound which still contains some of the deacetylated compound.

LC-MS (Method 1): Rₜ=0.91 min and 0.95 min; MS (ESIpos): m/z=528 and 570 (M+H)⁺.

### Intermediate C102

### (2S)-4-[{(1R)-1-[1-Benzyl-4-(2,5-difluorophenyl)-1H-pyrrol-2-yl]-2,2-dimethylpropyl}(glycoloyl)amino]-2-{[(benzyloxy)carbonyl]amino}butanoic acid

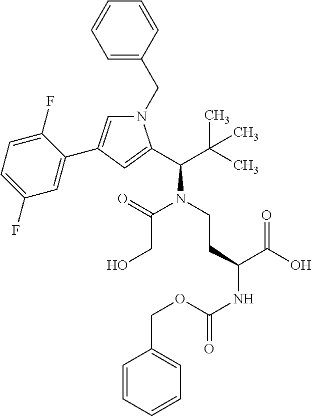 [SMILES: CC(C)(C)[C@H](c1cc(-c2cc(F)ccc2F)cn1Cc1ccccc1)N(CC[C@H](NC(=O)OCc1ccccc1)C(=O)O)C(=O)CO]

First, intermediate C52 was subjected to a reductive alkylation with benzyl (2S)-2-{[(benzyloxy)carbonyl]amino}-4-oxobutanoate analogously to intermediate C2. Subsequently, the secondary amino group was acylated with 2-chloro-2-oxoethyl acetate, and finally the two ester groups were hydrolyzed using a 2M solution of lithium hydroxide in methanol.

LC-MS (Method 1): Rₜ=1.31 min; MS (ESIpos): m/z=646 (M−H)⁻.

### Intermediate C103

### 2-(Trimethylsilyl)ethyl N-[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]-N2-{[2-(trimethylsilyl) ethoxy]carbonyl}-L-glutaminate

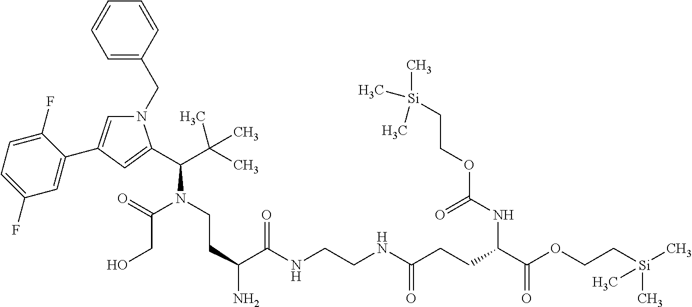 [SMILES: CC(C)(C)[C@H](c1cc(-c2cc(F)ccc2F)cn1Cc1ccccc1)N(CC[C@H](N)C(=O)NCCNC(=O)CC[C@H](NC(=O)OCC[Si](C)(C)C)C(=O)OCC[Si](C)(C)C)C(=O)CO]

The title compound was prepared by initially coupling 151 mg (0.23 mmol) of intermediate C102 with 128 mg (0.234 mmol) of intermediate L98 in DMF in the presence of HATU and N,N-diisopropylethylamine. The Z protective group was then removed by hydrogenating over 10% palladium on activated carbon at RT at standard hydrogen pressure for 30 minutes, giving the title compound.

Yield: 30% of theory over 2 steps

LC-MS (Method 1): Rₜ=1.14 min; MS (ESIpos): m/z=929 (M+H)⁺.

### Intermediate C104

### 2-(Trimethylsilyl)ethyl (3R,4R)-3-[({(1R)-1-[1-benzyl-4-(2,5-difluorophenyl)-1H-pyrrol-2-yl]-2,2-dimethylpropyl}amino)methyl]-4-fluoropyrrolidine-1-carboxylate

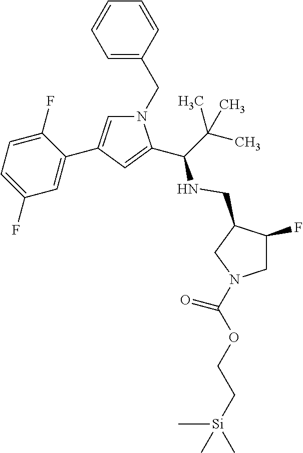 [SMILES: CC(C)(C)[C@@H](NC[C@@H]1CN(C(=O)OCC[Si](C)(C)C)C[C@@H]1F)c1cc(-c2cc(F)ccc2F)cn1Cc1ccccc1]

1.87 g (8.84 mmol) of sodium triacetoxyborohydride were added to a solution of 2.24 g (6.31 mmol) of (1R)-1-[1-benzyl-4-(2,5-difluorophenyl)-1H-pyrrol-2-yl]-2,2-dimethylpropane-1-amine in 56.0 ml of dichloromethane with molecular sieve 4 Å, and the mixture was stirred at room temperature for 15 minutes. 2.20 g (7.58 mmol) of 2-(trimethylsilyl)ethyl (3R,4S)-3-fluoro-4-formylpyrrolidine-1-carboxylate (lit: WO 2014/151030A1) were then added, and the reaction mixture was stirred at room temperature for 3.5 h. The mixture was diluted with dichloromethane and the organic phase was washed with sat. sodium bicarbonate solution and water. The organic phase was dried over sodium sulphate and concentrated. The residue was purified by prep. HPLC. This gave 1.39 g (24% of theory) of the title compound.

LC-MS (Method 1): Rₜ=1.15 min; MS (ESIpos): m/z=600 (M+H)⁺.

### Intermediate C105

### 2-(Trimethylsilyl)ethyl (3R,4R)-3-{[{(1R)-1-[1-benzyl-4-(2,5-difluorophenyl)-1H-pyrrol-2-yl]-2,2-dimethylpropyl}(chloroacetyl)amino]methyl}-4-fluoropyrrolidine-1-carboxylate

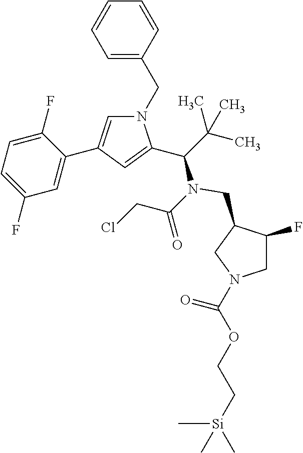 [SMILES: CC(C)(C)[C@H](c1cc(-c2cc(F)ccc2F)cn1Cc1ccccc1)N(C[C@@H]1CN(C(=O)OCC[Si](C)(C)C)C[C@@H]1F)C(=O)CCl]

295.0 mg (2.91 mmol) of triethylamine and 418.9 mg (3.71 mmol) of chloroacetyl chloride were added to a solution of 692.8 mg (0.88 mmol) of 2-(trimethylsilyl)ethyl (3R,4R)-3-[({(1R)-1-[1-benzyl-4-(2,5-difluorophenyl)-1H-pyrrol-2-yl]-2,2-dimethylpropyl}amino)methyl]-4-fluoropyrrolidine-1-carboxylate (Intermediate C104) in 8.7 ml of dichloromethane with molecular sieve 4 Å, and the reaction mixture was stirred at RT for 2.5 h. The reaction mixture was diluted with dichloromethane and the organic phase was washed with sat. sodium bicarbonate solution and sat. ammonium chloride solution. The organic phase was dried over sodium sulphate and concentrated. Once more, 295.0 mg (2.91 mmol) of triethylamine and 418.9 mg (3.71 mmol) of chloroacetyl chloride were added to the residue in 8.7 ml of dichloromethane with molecular sieve 4 Å, and the reaction mixture was stirred at RT for 3 h. The reaction mixture was diluted with dichloromethane and the organic phase was washed with sat. sodium bicarbonate solution and sat. ammonium chloride solution. The organic phase was dried over sodium sulphate and concentrated. The organic phase was dried over sodium sulphate, concentrated and used further without purification. This gave 691 mg (74% of theory, 64% pure) of the title compound.

LC-MS (Method 1): Rₜ=1.78 min; MS (ESIpos): m/z=676 (M+H)⁺.

### Intermediate C106

### 3-{[2-({(1R)-1-[1-Benzyl-4-(2,5-difluorophenyl)-1H-pyrrol-2-yl]-2,2-dimethylpropyl}{[(3R,4R)-4-fluoro-1-{[2-(trimethylsilyl)ethoxy]carbonyl}pyrrolidin-3-yl]methyl}amino)-2-oxoethyl]sulphanyl}propanoic acid

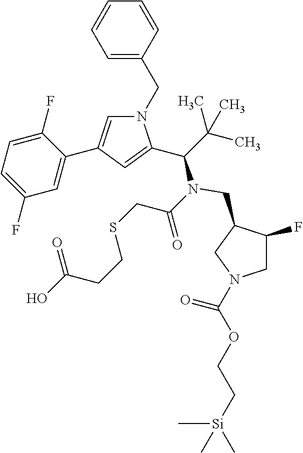 [SMILES: CC(C)(C)[C@H](c1cc(-c2cc(F)ccc2F)cn1Cc1ccccc1)N(C[C@@H]1CN(C(=O)OCC[Si](C)(C)C)C[C@@H]1F)C(=O)CSCCC(=O)O]

316 mg (2.29 mmol) of potassium carbonate were added to a mixture of 691.0 mg (0.65 mmol) of 2-(trimethylsilyl)ethyl (3R,4R)-3-{[{(1R)-1-[1-benzyl-4-(2,5-difluorophenyl)-1H-pyrrol-2-yl]-2,2-dimethylpropyl}(chloroacetyl)amino]methyl}-4-fluoropyrrolidine-1-carboxylate (intermediate C105) and 76.3 mg (0.72 mmol) of 3-sulphanylpropanoic acid in 15 ml of methanol and a few drops of water. The reaction mixture was stirred at 50° C. for 1.5 h. Ethyl acetate was added to the reaction mixture, and the org. phase was washed repeatedly with water and with sat. NaCl solution. The organic phase was dried over magnesium sulphate and the solvent was evaporated under reduced pressure and the residue was dried under high vacuum. The residue was used further without work-up. This gave 502 mg (67% of theory, 65% pure) of the title compound.

LC-MS (Method 1): Rₜ=1.48 min; MS (ESIneg): m/z=744 (M−H)⁻.

### Intermediate C107

### S-[2-({(1R)-1-[1-Benzyl-4-(2,5-difluorophenyl)-1H-pyrrol-2-yl]-2,2-dimethylpropyl}{[(3R,4R)-4-fluoro-1-{[2-(trimethylsilyl)ethoxy]carbonyl}pyrrolidin-3-yl]methyl}amino)-2-oxoethyl]-L-cysteine

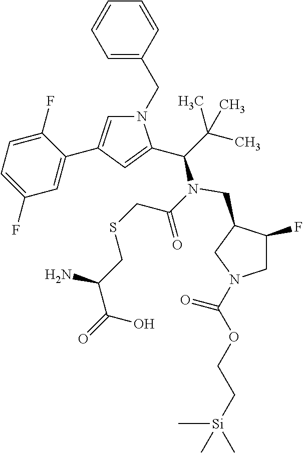 [SMILES: CC(C)(C)[C@H](c1cc(-c2cc(F)ccc2F)cn1Cc1ccccc1)N(C[C@@H]1CN(C(=O)OCC[Si](C)(C)C)C[C@@H]1F)C(=O)CSC[C@H](N)C(=O)O]

203.6 mg (1.68 mmol) of L-cysteine together with 201.7 mg (2.40 mmol) of sodium bicarbonate were suspended in 0.95 ml of water. 170.0 mg (0.24 mmol) of 2-(trimethylsilyl)ethyl (3R,4R)-3-{[{(1R)-1-[1-benzyl-4-(2,5-difluorophenyl)-1H-pyrrol-2-yl]-2,2-dimethylpropyl}(chloroacetyl)amino]methyl}-4-fluoropyrrolidine-1-carboxylate (intermediate 105), dissolved in 9.5 ml of isopropanol, and 438.5 mg (2.40 mmol) and 1,8-diazabicyclo[5.4.0]undec-7-ene were added. The reaction mixture was stirred at 50° C. for 3 h. Ethyl acetate was added to the mixture and the org. phase was washed repeatedly with sat. sodium bicarbonate solution and with sat. NaCl solution. The organic phase was dried over sodium sulphate and the solvent was evaporated under reduced pressure. The residue was used further without further purification. This gave 152 mg (83% of theory) of the title compound.

LC-MS (Method 1): Rₜ=1.26 min; MS (ESIpos): m/z=762 (M+H)⁺.

### Intermediate C115

### N-[6-(2,5-Dioxo-2,5-dihydro-1H-pyrrol-1-yl)hexanoyl]-L-valyl-N-[3-({(1R)-1-[1-benzyl-4-(2,5-difluorophenyl)-1H-pyrrol-2-yl]-2,2-dimethylpropyl}{[(2-carboxyethyl)sulphanyl]acetyl}amino)propyl]-L-alaninamide

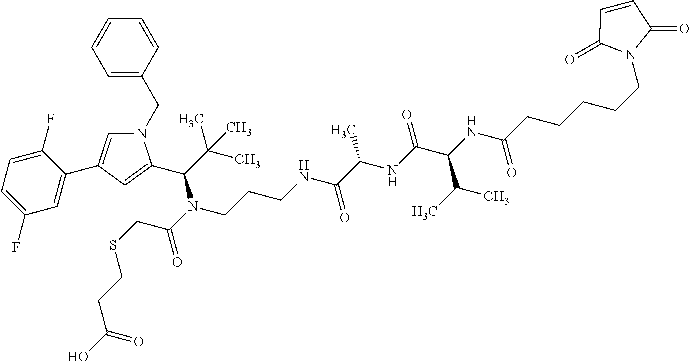 [SMILES: CC(C)[C@H](NC(=O)CCCCCN1C(=O)C=CC1=O)C(=O)N[C@@H](C)C(=O)NCCCN(C(=O)CSCCC(=O)O)[C@@H](c1cc(-c2cc(F)ccc2F)cn1Cc1ccccc1)C(C)(C)C]

11-{(1R)-1-[1-Benzyl-4-(2,5-difluorophenyl)-1H-pyrrol-2-yl]-2,2-dimethylpropyl}-2,2-dimethyl-6,12-dioxo-5-oxa-14-thia-7,11-diaza-2-silaheptadecan-17-oic acid (200 mg, 285 μmol) (intermediate C69) was dissolved in 10 ml of trifluoroethanol. Zinc chloride (233 mg, 1.71 mmol) was added and the reaction mixture was stirred at 50° C. for 1 hour. Two more times, zinc chloride (233 mg, 1.71 mmol) was added and the reaction mixture was stirred at 50° C. for 1 hour. Ethylenediamine-N,N,N′,N′-tetraacetic acid (1.50 g, 5.13 mmol) was added, followed by water (0.1% TFA), and the mixture was then concentrated under reduced pressure. The residue was purified by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gives 162 mg (85% of theory) of the compound 3-({2-[(3-aminopropyl) {(1R)-1-[1-benzyl-4-(2,5-difluorophenyl)-1H-pyrrol-2-yl]-2,2-dimethylpropyl}amino]-2-oxoethyl}sulphanyl)propanoic acid/trifluoroacetic acid (1:1).

LC-MS (Method 1): Rₜ=0.94 min; MS (ESIneg): m/z=556 [M−H]⁻.

3-({2-[(3-Aminopropyl) {(1R)-1-[1-benzyl-4-(2,5-difluorophenyl)-1H-pyrrol-2-yl]-2,2-dimethylpropyl}amino]-2-oxoethyl}sulphanyl)propanoic acid/trifluoro acetic acid (1:1) (80.0 mg, 119 μmol) was dissolved in 5.0 ml of DMF, and 2,5-dioxopyrrolidin-1-yl N-[6-(2,5-dioxo-2,5-dihydro-1H-pyrrol-1-yl)hexanoyl]-L-valyl-L-alaninate (69.4 mg, 82% pure, 119 μmol) (intermediate L88) and N,N-diisopropylethylamine (41 μl, 240 μmol) were added. The reaction mixture was stirred at RT for 2 h30, and water (0.1% TFA) was added. The mixture was concentrated and purified directly by prep. RP-HPLC (column: Reprosil 250×30; 10μ, flow rate: 50 ml/min, MeCN/water, 0.1% TFA). The solvent was evaporated under reduced pressure and the residue was dried under high vacuum. This gave 82.2 mg (75% of theory) of the title compound. LC-MS (Method 1): Rₜ=1.17 min; MS (ESIpos): m/z=921 [M+H]⁺

### Intermediate C116

### N-[6-(2,5-Dioxo-2,5-dihydro-1H-pyrrol-1-yl)hexanoyl]-L-valyl-N-[3-({(1R)-1-[1-benzyl-4-(2,5-difluorophenyl)-1H-pyrrol-2-yl]-2,2-dimethylpropyl}[({3-[(2-carboxyethyl)amino]-3-oxopropyl}sulphanyl) acetyl]amino)propy 1]-L-alaninamide

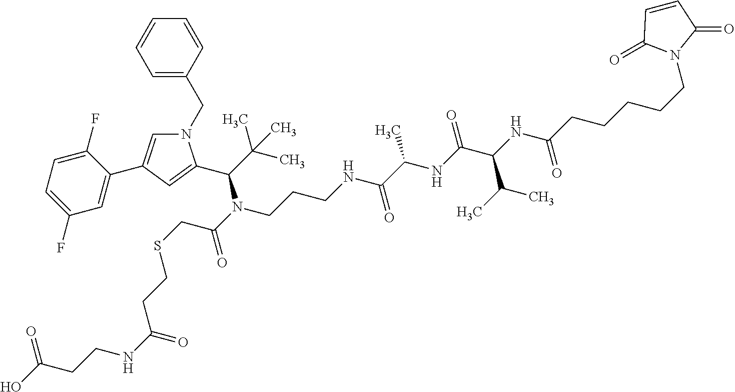 [SMILES: CC(C)[C@H](NC(=O)CCCCCN1C(=O)C=CC1=O)C(=O)N[C@@H](C)C(=O)NCCCN(C(=O)CSCCC(=O)NCCC(=O)O)[C@@H](c1cc(-c2cc(F)ccc2F)cn1Cc1ccccc1)C(C)(C)C]

Under argon, N-[6-(2,5-dioxo-2,5-dihydro-1H-pyrrol-1-yl)hexanoyl]-L-valyl-N-[3-({(1R)-1-[1-benzyl-4-(2,5-difluorophenyl)-1H-pyrrol-2-yl]-2,2-dimethylpropyl}{[(2-carboxyethyl)sulphanyl]acetyl}amino)propyl]-L-alaninamide (56.7 mg, 61.6 μmol) (intermediate C115) and tert-butyl beta-alaninate hydrochloride (1:1) (13.4 mg, 73.9 μmol) were initially charged in 3.0 ml of DMF, and HATU (28.1 mg, 73.9 μmol) and N,N-diisopropylethylamine (32 μl, 180 μmol) were added. The reaction mixture was stirred at RT for 10 min and then purified directly by prep. RP-HPLC (column: Reprosil 250×30; 10μ, flow rate: 50 ml/min, MeCN/water). The solvents were evaporated under reduced pressure and the residue was dried under high vacuum. This gave 41.4 mg (64% of theory) of the compound N-[6-(2,5-dioxo-2,5-dihydro-1H-pyrrol-1-yl)hexanoyl]-L-valyl-N-(14-{(1R)-1-[1-benzyl-4-(2,5-difluorophenyl)-1H-pyrrol-2-yl]-2,2-dimethylpropyl}-2,2-dimethyl-4,8,13-trioxo-3-oxa-11-thia-7,14-diazaheptadecan-17-yl)-L-alaninamide.

LC-MS (Method 1): Rₜ=1.28 min; MS (ESIpos): m/z=1048 [M+H]⁺

N-[6-(2,5-Dioxo-2,5-dihydro-1H-pyrrol-1-yl)hexanoyl]-L-valyl-N-(14-{(1R)-1-[1-benzyl-4-(2,5-difluorophenyl)-1H-pyrrol-2-yl]-2,2-dimethylpropyl}-2,2-dimethyl-4,8,13-trioxo-3-oxa-11-thia-7,14-diazaheptadecan-17-yl)-L-alaninamide (39.3 mg, 37.5 μmol) was dissolved in 2.5 ml of trifluoroethanol. Zinc chloride (30.7 mg, 225 μmol) was added and the reaction mixture was stirred at 50° C. for 1 hour. Once more, zinc chloride (30.7 mg, 225 μmol) was added and the reaction mixture was stirred at 50° C. for 1 hour. Ethylenediamine-N,N,N′,N′-tetraacetic acid (131 mg, 450 μmol) was added, followed by water (0.1% TFA), and the mixture was then concentrated under reduced pressure. The residue was purified by prep. RP-HPLC (column: Reprosil 250×30; 10μ, flow rate: 50 ml/min, MeCN/water, 0.1% TFA). The solvents were evaporated under reduced pressure and the residue was taken up in a little water and lyophilized. This gave 30 mg (81% of theory) of the title compound.

LC-MS (Method 1): Rₜ=1.12 min; MS (ESIpos): m/z=992 [M+H]⁺

### Intermediate L1

### Trifluoroacetic acid/N-(2-aminoethyl)-2-(2,5-dioxo-2,5-dihydro-1H-pyrrol-1-ylacetamide (1:1)

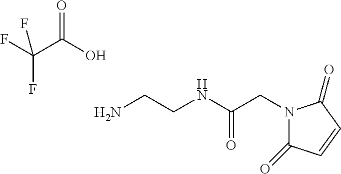 [SMILES: O=C(O)C(F)(F)F ; NCCNC(=O)CN1C(=O)C=CC1=O]

The title compound was prepared by classical methods of peptide chemistry from commercially available (2,5-dioxo-2,5-dihydro-1H-pyrrol-1-ylacetic acid and tert-butyl (2-aminoethyl)carbamate.

HPLC (Method 11): Rₜ=0.19 min;

LC-MS (Method 1): Rₜ=0.17 min; MS (ESIpos): m/z=198 (M+H)⁺.

### Intermediate L2

### Trifluoroacetic acid/rel-(1R,2S)-2-amino-N-[2-(2,5-dioxo-2,5-dihydro-1H-pyrrol-1-yl)ethyl]-cyclopentanecarboxamide (1:1)

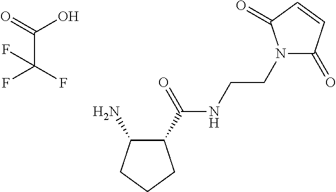 [SMILES: O=C(O)C(F)(F)F ; N[C@H]1CCC[C@H]1C(=O)NCCN1C(=O)C=CC1=O]

The title compound was prepared from 50 mg (0.214 mmol) of commercially available cis-2-[(tert-butoxycarbonyl)amino]-1-cyclopentanecarboxylic acid and 60 mg (0.235 mmol) of likewise commercially available trifluoroacetic acid/1-(2-aminoethyl)-1H-pyrrole-2,5-dione (1:1) by coupling with EDC/HOBT and subsequent deprotection with TFA. This gave 36 mg (38% of theory over 2 steps) of the title compound.

HPLC (Method 11): Rₜ=0.2 min;

LC-MS (Method 1): Rₜ=0.17 min; MS (ESIpos): m/z=252 (M+H)⁺.

### Intermediate L3

### Trifluoroacetic acid/(1 S,2R)-2-amino-N-[2-(2,5-dioxo-2,5-dihydro-1H-pyrrol-1-yl)ethyl]cyclopentanecarboxamide (1:1)

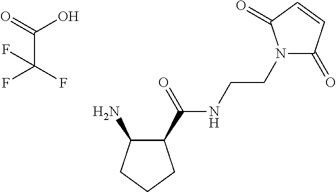 [SMILES: O=C(O)C(F)(F)F ; N[C@@H]1CCC[C@@H]1C(=O)NCCN1C(=O)C=CC1=O]

The title compound was prepared from 50 mg (0.214 mmol) of commercially available (1S,2R)-2-[(tert-butoxycarbonyl)amino]cyclopentanecarboxylic acid with 72 mg (0.283 mmol) of likewise commercially available trifluoroacetic acid/1-(2-aminoethyl)-1H-pyrrole-2,5-dione (1:1) by coupling with EDC/HOBT and subsequent deprotection with TFA. This gave 13 mg (16% of theory over 2 steps) of the title compound.

HPLC (Method 11): Rₜ=0.2 min;

LC-MS (Method 1): Rₜ=0.2 min; MS (ESIpos): m/z=252 (M+H)⁺.

### Intermediate L4

### Trifluoroacetic acid/N-(2-aminoethyl)-4-(2,5-dioxo-2,5-dihydro-1H-pyrrol-1-yl)cyclohexanecarboxamide (1:1)

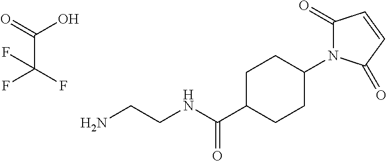 [SMILES: O=C(O)C(F)(F)F ; NCCNC(=O)C1CCC(N2C(=O)C=CC2=O)CC1]

The title compound was prepared by classical methods of peptide chemistry from commercially available l-[(4-{[(2,5-dioxopyrrolidin-1-yl)oxy]carbonyl}cyclohexyl)methyl]-1H-pyrrole-2,5-dione and tert-butyl (2-aminoethyl)carbamate.

HPLC (Method 11): Rₜ=0.26 min;

LC-MS (Method 1): Rₜ=0.25 min; MS (ESIpos): m/z=280 (M+H)⁺.

### Intermediate L5

### Trifluoroacetic acid/N-[4-(2,5-dioxo-2,5-dihydro-1H-pyrrol-1-yl)phenyl]-beta-alaninamide (1:1)

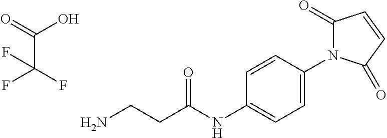 [SMILES: O=C(O)C(F)(F)F ; NCCC(=O)Nc1ccc(N2C(=O)C=CC2=O)cc1]

The title compound was prepared by classical methods of peptide chemistry from commercially available 1-(4-aminophenyl)-1H-pyrrole-2,5-dione and N-(tert-butoxycarbonyl)-beta-alanine.

HPLC (Method 11): Rₜ=0.22 min;

LC-MS (Method 1): Rₜ=0.22 min; MS (ESIpos): m/z=260 (M+H)⁺.

### Intermediate L6

### Trifluoroacetic acid/tert-butyl-N-[6-(2,5-dioxo-2,5-dihydro-1H-pyrrol-1-yl)hexanoyl]-L-valyl-L-alanyl-L-lysinate (1:1)

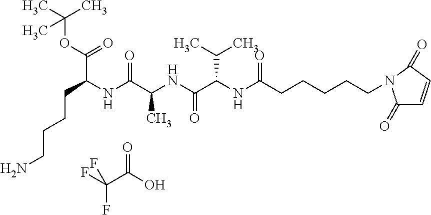 [SMILES: CC(C)[C@H](NC(=O)CCCCCN1C(=O)C=CC1=O)C(=O)N[C@@H](C)C(=O)N[C@@H](CCCCN)C(=O)OC(C)(C)C ; O=C(O)C(F)(F)F]

The title compound was prepared by initially coupling, in the presence of EDC/HOBT, commercially available 6-(2,5-dioxo-2,5-dihydro-1H-pyrrol-1-yl)hexanoic acid with the partially protected peptide tert-butyl L-valyl-L-alanyl-N⁶-(tert-butoxycarbonyl)-L-lysinate, prepared by classical methods of peptide chemistry. This was followed by deprotection at the amino group under gentle conditions by stirring in 5% strength trifluoroacetic acid in DCM at RT, which gave the title compound in a yield of 37%.

HPLC (Method 11): Rₜ=1.29 min;

LC-MS (Method 1): Rₜ=0.62 min; MS (ESIpos): m/z=566 (M+H)⁺.

### Intermediate L7

### Trifluoroacetic acid/beta-alanyl-L-valyl-N⁵-carbamoyl-N-[4-(2,5-dioxo-2,5-dihydro-1H-pyrrol-1-yl)phenyl]-L-ornithinamide (1:1)

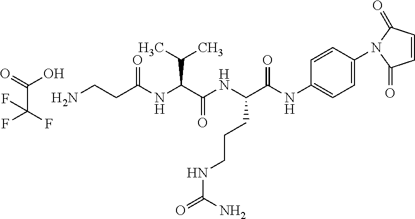

The title compound was prepared according to classical methods of peptide chemistry from commercially available 1-(4-aminophenyl)-1H-pyrrole-2,5-dione by sequential coupling with N²-(tert-butoxycarbonyl)-N⁵-carbamoyl-L-ornithine in the presence of HATU, deprotection with TFA, coupling with 2,5-dioxopyrrolidin-1-yl N-(tert-butoxycarbonyl)-L-valinate, deprotection with TFA, coupling with 2,5-dioxopyrrolidin-1-yl N-(tert-butoxycarbonyl)-beta-alaninate and another deprotection with TFA. 32 mg of the title compound were obtained.

HPLC (Method 11): Rₜ=0.31 min;

LC-MS (Method 1): Rₜ=0.47 min; MS (ESIpos): m/z=516 (M+H)⁺.

### Intermediate L8

### Trifluoroacetic acid/F-alanyl-N5-carbamoyl-N-[4-(2,5-dioxo-2,5-dihydro-1H-pyrrol-1-yl)phenyl]-L-ornithinamide (1:1)

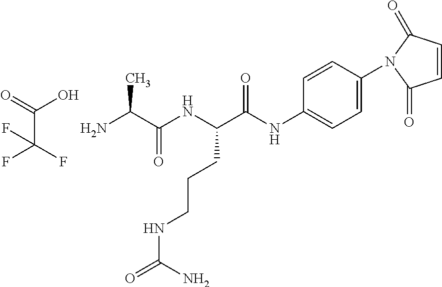 [SMILES: O=C(O)C(F)(F)F ; C[C@H](N)C(=O)N[C@@H](CCCNC(N)=O)C(=O)Nc1ccc(N2C(=O)C=CC2=O)cc1]

The title compound was prepared according to classical methods of peptide chemistry from commercially available 1-(4-aminophenyl)-1H-pyrrole-2,5-dione by sequential coupling with N²-(tert-butoxycarbonyl)-N⁵-carbamoyl-L-ornithine in the presence of HATU, deprotection with TFA, coupling with 2,5-dioxopyrrolidin-1-yl N-(tert-butoxycarbonyl)-L-alaninate and another deprotection with TFA. 171 mg of the title compound were obtained.

HPLC (Method 11): Rₜ=0.23 min;

LC-MS (Method 7): Rₜ=0.3 min; MS (ESIpos): m/z=417 (M+H)⁺.

### Intermediate L9

### Trifluoroacetic acid/beta-alanyl-L-valyl-N⁵-carbamoyl-N-[4-(2-methoxy-2-oxoethyl)phenyl]-L-ornithinamide (1:1)

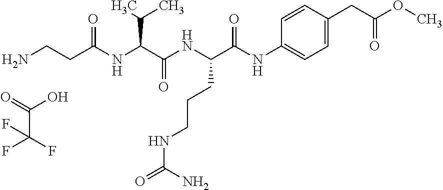 [SMILES: O=C(O)C(F)(F)F ; COC(=O)Cc1ccc(NC(=O)[C@H](CCCNC(N)=O)NC(=O)[C@@H](NC(=O)CCN)C(C)C)cc1]

The title compound was prepared analogously to Intermediate L7 from commercially available methyl (4-aminophenyl)acetate. 320 mg of the title compound were obtained.

HPLC (Method 11): Rₜ=0.45 min;

LC-MS (Method 1): Rₜ=0.48 min; MS (ESIpos): m/z=493 (M+H)⁺.

### Intermediate L10

### N-[6-(2,5-Dioxo-2,5-dihydro-1H-pyrrol-1-yl)hexanoyl]-L-valyl-L-alanyl-rel-N⁶-{[(1R,2S)-2-aminocyclopentyl]carbonyl}-L-lysine/trifluoroacetic acid (1:2)

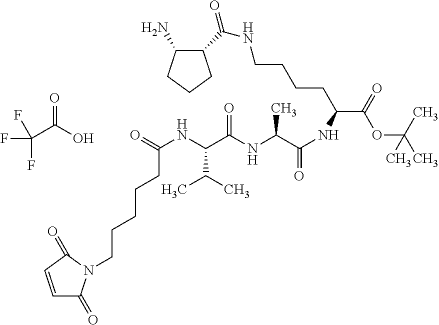 [SMILES: O=C(O)C(F)(F)F ; CC(C)[C@H](NC(=O)CCCCCN1C(=O)C=CC1=O)C(=O)N[C@@H](C)C(=O)N[C@@H](CCCCNC(=O)[C@@H]1CCC[C@@H]1N)C(=O)OC(C)(C)C]

The title compound was prepared from Intermediate L6 by coupling with cis-2-[(tert-butoxycarbonyl)amino]-1-cyclopentanecarboxylic acid with EDC/HOBT and subsequent deprotection with TFA. This gave 12 mg (52% of theory over 2 steps) of the title compound.

HPLC (Method 11): Rₜ=1.45 min;

LC-MS (Method 1): Rₜ=0.73 min; MS (ESIpos): m/z=677 (M+H)⁺.

### Intermediate L11

### N-[6-(2,5-Dioxo-2,5-dihydro-1H-pyrrol-1-yl)hexanoyl]-L-valyl-L-alanyl-N⁶-{[(1S,2R)-2-aminocyclopentyl]carbonyl}-L-lysine/trifluoroacetic acid (1:2)

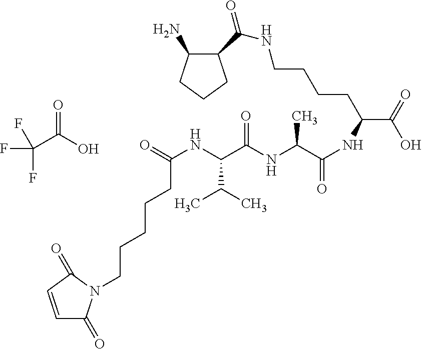 [SMILES: O=C(O)C(F)(F)F ; CC(C)[C@H](NC(=O)CCCCCN1C(=O)C=CC1=O)C(=O)N[C@@H](C)C(=O)N[C@@H](CCCCNC(=O)[C@H]1CCC[C@H]1N)C(=O)O]

The title compound was prepared from Intermediate L6 by coupling with (1S,2R)-2-[(tert-butoxycarbonyl)amino]cyclopentanecarboxylic acid with EDC/HOBT and subsequent deprotection with TFA. This gave 11 mg (39% of theory over 2 steps) of the title compound.

HPLC (Method 11): Rₜ=1.45 min;

LC-MS (Method 1): Rₜ=0.74 min; MS (ESIpos): m/z=677 (M+H)⁺.

### Intermediate L12

### Trifluoroacetic acid/1-[2-(2-aminoethoxy)ethyl]-1H-pyrrole-2,5-dione (1:1)

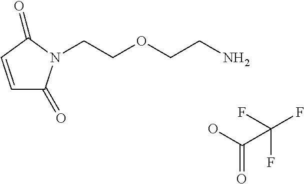 [SMILES: NCCOCCN1C(=O)C=CC1=O ; O=C(O)C(F)(F)F]

381 mg (2.46 mmol) of methyl 2,5-dioxo-2,5-dihydro-1H-pyrrole-1-carboxylate were added to 228 mg (1.12 mmol) of tert-butyl [2-(2-aminoethoxy)ethyl]carbamate dissolved in 7 ml of dioxane/water 1:1. 1.2 ml of a saturated sodium bicarbonate solution were then added and the reaction was stirred at RT. After a total of 5 days of stirring and 2 further additions of the same amounts of the sodium bicarbonate solution, the reaction was worked up by acidification with trifluoroacetic acid, concentration on a rotary evaporator and purification of the residue by preparative HPLC. The appropriate fractions were combined, the solvent was removed under reduced pressure and the residue was lyophilized from acetonitrile/water 1:1.

The residue was taken up in 3 ml of dichloromethane, and 1 ml of trifluoroacetic acid was added. After 15 min of stirring at RT, the solvent was removed under reduced pressure and the residue was lyophilized from acetonitrile/water 1:1. This gave 70 mg (67% of theory over 2 steps) of the title compound as a resinous residue.

HPLC (Method 11): Rₜ=0.2 min;

LC-MS (Method 1): Rₜ=0.18 min; MS (ESIpos): m/z=185 (M+H)⁺.

### Intermediate L13

### Trifluoroacetic acid/tert-butyl N²-[(2,5-dioxo-2,5-dihydro-1H-pyrrol-1-ylacetyl]-L-lysinate (1:1)

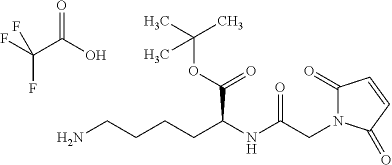 [SMILES: O=C(O)C(F)(F)F ; CC(C)(C)OC(=O)[C@H](CCCCN)NC(=O)CN1C(=O)C=CC1=O]

The title compound was prepared by coupling of (2,5-dioxo-2,5-dihydro-1H-pyrrol-1-ylacetic acid with tert-butyl N⁶-(tert-butoxycarbonyl)-L-lysinate hydrochloride (1:1) in the presence of EDC/HOBT and subsequent gentle removal of the tert-butoxycarbonyl protective group analogously to Intermediate L6.

HPLC (Method 11): Rₜ=0.42 min;

LC-MS (Method 1): Rₜ=0.43 min; MS (ESIpos): m/z=340 (M+H)⁺.

### Intermediate L14

### Trifluoroacetic acid/1-[2-(4-aminopiperazin-1-yl)-2-oxoethyl]-1H-pyrrole-2,5-dione (1:1)

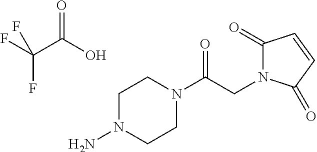 [SMILES: O=C(O)C(F)(F)F ; NN1CCN(C(=O)CN2C(=O)C=CC2=O)CC1]

The title compound was prepared analogously to Intermediate L2 over 2 steps from tert-butyl piperazin-1-ylcarbamate and (2,5-dioxo-2,5-dihydro-1H-pyrrol-1-yl)acetic acid.

HPLC (Method 11): Rₜ=0.2 min;

LC-MS (Method 3): Rₜ=0.25 min; MS (ESIpos): m/z=239 (M+H)⁺.

### Intermediate L15

### Trifluoroacetic acid/N-(2-aminoethyl)-3-(2-{2-[2-(2,5-dioxo-2,5-dihydro-1H-pyrrol-1-yl)ethoxy]ethoxy}ethoxy)propanamide (1:1)

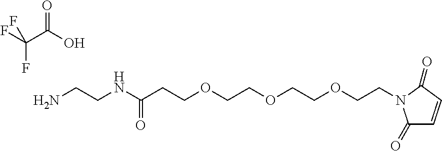 [SMILES: O=C(O)C(F)(F)F ; NCCNC(=O)CCOCCOCCOCCN1C(=O)C=CC1=O]

2.93 g (10.58 mmol) of tert-butyl 3-{2-[2-(2-aminoethoxy)ethoxy]ethoxy}propanoate were dissolved in 100 ml of dioxane/water 1:1, and 3.28 g (21.15 mmol) of methyl 2,5-dioxo-2,5-dihydro-1H-pyrrole-1-carboxylate and a saturated sodium bicarbonate solution were added until a pH of 6-7 had been reached. The solution was stirred at RT for 30 min and the 1,4-dioxane was then evaporated under reduced pressure. 200 ml of water were then added, and the mixture was extracted three times with in each case 300 ml of ethyl acetate. The organic extracts were combined, dried over magnesium sulphate and filtered. Concentration gave tert-butyl 3-(2-{2-[2-(2,5-dioxo-2,5-dihydro-1H-pyrrol-1-yl)ethoxy]ethoxy}ethoxypropanoate as a brown oil which was then dried under high vacuum.

HPLC (Method 11): Rₜ=1.5 min;

LC-MS (Method 3): Rₜ=0.88 min; MS (ESIpos): m/z=375 (M+NH₄)⁺.

This intermediate was converted by standard methods (deprotection with TFA, coupling with tert-butyl (2-aminoethyl)carbamate and another deprotection with TFA) into the title compound.

HPLC (Method 11): Rₜ=0.2 min;

LC-MS (Method 3): Rₜ=0.25 min; MS (ESIpos): m/z=344 (M+H)⁺.

### Intermediate L16

### N-[6-(2,5-Dioxo-2,5-dihydro-1H-pyrrol-1-yl)hexanoyl]-L-valyl-N⁵-carbamoyl-L-ornithine

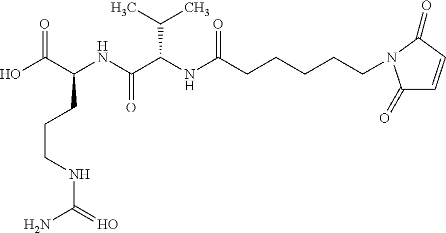 [SMILES: CC(C)[C@H](NC(=O)CCCCCN1C(=O)C=CC1=O)C(=O)N[C@@H](CCCNC(N)=O)C(=O)O]

535 mg (1.73 mmol) of commercially available 1-{6-[(2,5-dioxopyrrolidin-1-yl)oxy]-6-oxohexyl}-1H-pyrrole-2,5-dione and 930 ml of N,N-diisopropylethylamine were added to a solution of 266 mg (1.33 mmol) of L-valyl-N5-carbamoyl-L-ornithine in 24 ml of DMF. The reaction was treated in an ultrasonic bath for 24 h and then concentrated to dryness under reduced pressure. The residue that remained was purified by preparative HPCL and gave, after concentration of the appropriate fractions and drying of the residue under high vacuum, 337 mg (50% of theory) of the title compound.

HPLC (Method 11): Rₜ=0.4 min;

LC-MS (Method 3): Rₜ=0.58 min; MS (ESIpos): m/z=468 (M+H)⁺.

### Intermediate L17

### Trifluoroacetic acid/tert-butyl N-[6-(2,5-dioxo-2,5-dihydro-1H-pyrrol-1-yl)hexanoyl]-L-valyl-N⁵-carbamoyl-L-ornithyl-L-lysinate (1:1)

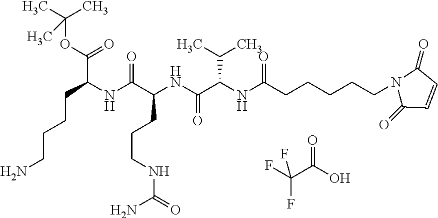 [SMILES: CC(C)[C@H](NC(=O)CCCCCN1C(=O)C=CC1=O)C(=O)N[C@@H](CCCNC(N)=O)C(=O)N[C@@H](CCCCN)C(=O)OC(C)(C)C ; O=C(O)C(F)(F)F]

The title compound was prepared by initially coupling 172 mg (0.37 mmol) of Intermediate L16 and 125 mg (0.37 mmol) of tert-butyl N6-(tert-butoxycarbonyl)-L-lysinate hydrochloride (1:1) in the presence of EDC/HOBT and N,N-diisopropylethylamine and then deprotecting the amino group under gentle conditions by stirring for 2 h in 10% strength trifluoroacetic acid in DCM at RT. Freeze-drying from acetonitrile/water gave 194 mg (49% of theory) of the title compound over 2 steps.

HPLC (Method 11): Rₜ=1.1 min;

LC-MS (Method 1): Rₜ=0.58 min; MS (ESIpos): m/z=652 (M+H)⁺.

### Intermediate L18

### Trifluoroacetic acid/beta-alanyl-L-alanyl-N⁵-carbamoyl-N-[4-(2-methoxy-2-oxoethyl)phenyl]-L-ornithinamide (1:1)

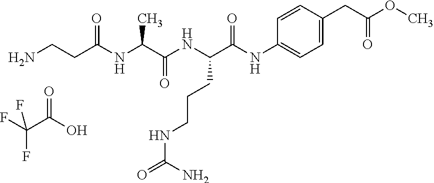 [SMILES: O=C(O)C(F)(F)F ; COC(=O)Cc1ccc(NC(=O)[C@H](CCCNC(N)=O)NC(=O)[C@H](C)NC(=O)CCN)cc1]

The title compound was prepared from methyl (4-aminophenyl)acetate analogously to Intermediate L7 sequentially according to classical methods of peptide chemistry by linking N²-(tert-butoxycarbonyl)-N⁵-carbamoyl-L-ornithine in the presence of HATU, deprotection with TFA, coupling with 2,5-dioxopyrrolidin-1-yl N-(tert-butoxycarbonyl)-L-alaninate, deprotection with TFA, coupling with 2,5-dioxopyrrolidin-1-yl N-(tert-butoxycarbonyl)-beta-alaninate and another deprotection with TFA. 330 mg of the title compound were obtained.

HPLC (Method 11): Rₜ=0.29 min;

LC-MS (Method 1): Rₜ=0.41 min; MS (ESIpos): m/z=465 (M+H)⁺.

### Intermediate L19

### Trifluoroacetic acid/L-alanyl-N5-carbamoyl-N-(4-{[(2,5-dioxo-2,5-dihydro-1H-pyrrol-1-yl) acetyl]amino}phenyl)-L-ornithinamide (1:1)

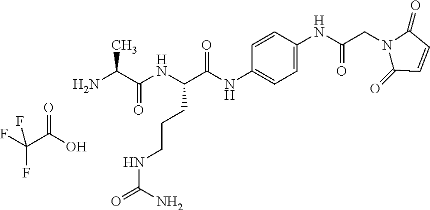 [SMILES: O=C(O)C(F)(F)F ; C[C@H](N)C(=O)N[C@@H](CCCNC(N)=O)C(=O)Nc1ccc(NC(=O)CN2C(=O)C=CC2=O)cc1]

The title compound was prepared from 1,4-phenylenediamine sequentially according to classical methods of peptide chemistry. In the first step, 942 mg (8.72 mmol) of 1,4-phenylenediamine were monoacylated with 0.8 g (2.9 mmol) of N²-(tert-butoxycarbonyl)-N⁵-carbamoyl-L-ornithine in the presence of HATU and N,N-diisopropylethylamine. In the second step, in an analogous manner, the second anilinic amino group was acylated with (2,5-dioxo-2,5-dihydro-1H-pyrrol-1-ylacetic acid in the presence of HATU and N,N-diisopropylethylamine. Deprotection with TFA, coupling with 2,5-dioxopyrrolidin-1-yl N-(tert-butoxycarbonyl)-L-alaninate and another deprotection with TFA then gave, in 3 further synthesis steps, the title compound, 148 mg of which were obtained by this route.

LC-MS (Method 1): Rₜ=0.21 min; MS (ESIpos): m/z=474 (M+H)⁺.

LC-MS (Method 4): Rₜ=0.2 min; MS (ESIpos): m/z=474 (M+H)⁺.

### Intermediate L20

### Trifluoroacetic acid/L-valyl-N⁵-carbamoyl-N-[4-(2,5-dioxo-2,5-dihydro-1H-pyrrol-1-ylphenyl]-L-ornithinamide (1:1)

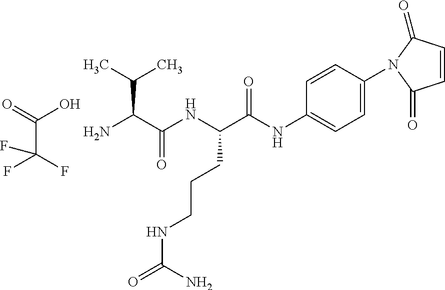 [SMILES: O=C(O)C(F)(F)F ; CC(C)[C@H](N)C(=O)N[C@@H](CCCNC(N)=O)C(=O)Nc1ccc(N2C(=O)C=CC2=O)cc1]

The title compound was prepared according to classical methods of peptide chemistry analogously to Intermediate L8 from commercially available 1-(4-aminophenyl)-1H-pyrrole-2,5-dione by sequential coupling with N²-(tert-butoxycarbonyl)-N⁵-carbamoyl-L-ornithine in the presence of HATU, deprotection with TFA, coupling with 2,5-dioxopyrrolidin-1-yl N-(tert-butoxycarbonyl)-L-valinate and another deprotection with TFA. 171 mg of the title compound were obtained.

HPLC (Method 11): Rₜ=0.28 min;

LC-MS (Method 1): Rₜ=0.39 min; MS (ESIpos): m/z=445 (M+H)⁺.

### Intermediate L21

### L-Valyl-N⁶-(tert-butoxycarbonyl)-N-[4-(2-methoxy-2-oxoethyl)phenyl]-L-lysinamide

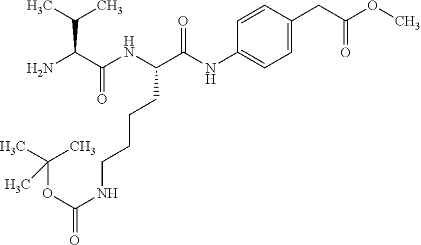 [SMILES: COC(=O)Cc1ccc(NC(=O)[C@H](CCCCNC(=O)OC(C)(C)C)NC(=O)[C@@H](N)C(C)C)cc1]

The title compound was prepared according to classical methods of peptide chemistry from commercially available 0.42 g (2.56 mmol) of methyl (4-aminophenyl)acetate by sequential coupling with N6-(tert-butoxycarbonyl)-N2-[(9H-fluoren-9-ylmethoxy)carbonyl]-L-lysine in the presence of HATU and N,N-diisopropylethylamine, deprotection with piperidine, coupling with 2,5-dioxopyrrolidin-1-yl N-[(benzyloxy)carbonyl]-L-valinate in the presence of N,N diisopropylethylamine and subsequent hydrogenolytic removal of the benzyloxycarbonyl protective group over 10% palladium on activated carbon. This gave 360 mg (32% of theory over 4 steps) of the title compound.

HPLC (Method 11): Rₜ=1.5 min;

LC-MS (Method 1): Rₜ=0.73 min; MS (ESIpos): m/z=493 (M+H)⁺.

### Intermediate L22

### Trifluoroacetic acid/N-[(9H-fluoren-9-ylmethoxy)carbonyl]-L-valyl-N-{4-[(2S)-2-amino-3-methoxy-3-oxopropyl]phenyl}-N⁵-carbamoyl-L-ornithinamide (1:1)

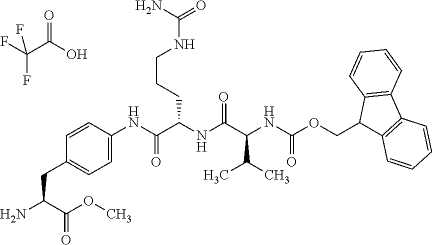 [SMILES: O=C(O)C(F)(F)F ; COC(=O)[C@@H](N)Cc1ccc(NC(=O)[C@H](CCCNC(N)=O)NC(=O)[C@@H](NC(=O)OCC2c3ccccc3-c3ccccc32)C(C)C)cc1]

The title compound was prepared from N-(tert-butoxycarbonyl)-4-nitro-L-phenylalanine sequentially according to classical methods of peptide chemistry. 2.5 g (8.06 mmol) of this starting material were in the first step initially converted into the caesium salt and then with iodomethane in DMF into the methyl ester.

Hydrogenolytically in methanol over 10% palladium on activated carbon, the nitro group was then converted into an amino group.

The amino group generated in this manner was then acylated with N5-carbamoyl-N2-[(9H-fluoren-9-ylmethoxy)carbonyl]-L-ornithine in DMF in the presence of HATU and N,N diisopropylethylamine. In the next step, the Fmoc group was removed with piperidine in DMF. Coupling was then carried out in DMF with N-[(9H-fluoren-9-ylmethoxy)carbonyl]-L-valine in the presence of 1-(3-dimethylaminopropyl)-3-ethylcarbodiimide hydrochloride, 1-hydroxy-1H-benzotriazole hydrate and N,N-diisopropylethylamine and finally removal of the tert-butoxycarbonyl group with trifluoroacetic acid.

HPLC (Method 11): Rₜ=1.6 min;

LC-MS (Method 1): Rₜ=0.77 min; MS (ESIpos): m/z=673 (M+H)⁺.

### Intermediate L23

### Trifluoroacetic acid/N-[2-(2,5-dioxo-2,5-dihydro-1H-pyrrol-1-yl)ethyl]-beta-alaninamide (1:1)

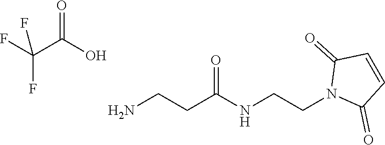 [SMILES: O=C(O)C(F)(F)F ; NCCC(=O)NCCN1C(=O)C=CC1=O]

The title compound was prepared from commercially available trifluoroacetic acid/1-(2-aminoethyl)-1H-pyrrole-2,5-dione (1:1) by coupling with N-(tert-butoxycarbonyl)-beta-alanine in the presence of EDCI/HOBT and N,N-diisopropylethylamine and subsequent deprotection with trifluoroacetic acid.

HPLC (Method 11): Rₜ=0.19 min.

### Intermediate L24

### Trifluoroacetic acid/l-amino-N-[2-(2,5-dioxo-2,5-dihydro-1H-pyrrol-1-yl)ethyl]cyclopropanecarboxamide (1:1)

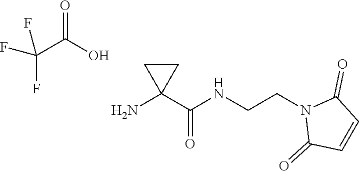 [SMILES: O=C(O)C(F)(F)F ; NC1(C(=O)NCCN2C(=O)C=CC2=O)CC1]

114 mg (0.67 mmol) of commercially available 1-[(tert-butoxycarbonyl)amino]cyclopropane-carboxylic acid were dissolved in 25 ml of DCM, 110 mg (0.623 mmol) of commercially available trifluoroacetic acid/1-(2-aminoethyl)-1H-pyrrole-2,5-dione (1:1) and 395 μl of N,N-diisopropylethylamine were added and the mixture was cooled to −10° C. 217 mg (0.793 mmol) of 2-bromo-1-ethylpyridinium tetrafluoroborate were then added, and the mixture was stirred at RT for 2 h. The mixture was then diluted with ethyl acetate and extracted successively with 10% strength citric acid, saturated sodium bicarbonate solution and saturated sodium chloride solution, then dried over magnesium sulphate and concentrated. Drying under high vacuum gave 152 mg of the protected intermediate.

These were then taken up in 10 ml of DCM and deprotected with 1 ml of trifluoroacetic acid. Lyophilization from acetonitrile/water gave 158 mg (71% of theory over 2 steps) of the title compound.

HPLC (Method 11): Rₜ=0.19 min.

LC-MS (Method 3): Rₜ=0.98 min; MS (ESIpos): m/z=224 (M+H)⁺.

### Intermediate L25

### N-[31-(2,5-Dioxo-2,5-dihydro-1H-pyrrol-1-yl)-29-oxo-4,7,10,13,16,19,22,25-octaoxa-28-azahentriacontan-1-oyl]-L-valyl-L-alanine

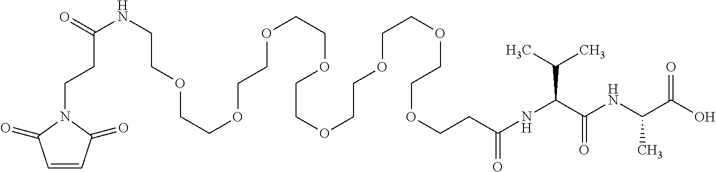 [SMILES: CC(C)[C@H](NC(=O)CCOCCOCCOCCOCCOCCOCCOCCOCCNC(=O)CCN1C(=O)C=CC1=O)C(=O)N[C@@H](C)C(=O)O]

31.4 mg (0.17 mmol) of valyl-L-alanine were dissolved in 3.0 ml of DMF, and 115.0 mg (0.17 mmol) of 3-(2,5-dioxo-2,5-dihydro-1H-pyrrol-1-yl)-N-{27-[(2,5-dioxopyrrolidin-1-yl)oxy]-27-oxo-3,6,9,12,15,18,21,24-octaoxaheptacos-1-yl}propanamide and 33.7 mg (0.33 mmol) of triethylamine were added. The mixture was stirred at RT overnight. The reaction mixture was purified directly by preparative RP-HPLC (column: Reprosil 250×30; 10μ, flow rate: 50 ml/min, MeCN/water). The solvents were evaporated under reduced pressure and the residue was dried under high vacuum. This gave 74.1 mg (58% of theory) of the title compound.

LC-MS (Method 1): Rₜ=0.61 min; MS (ESIpos): m/z=763 [M+H]⁺.

### Intermediate L26

### L-Valyl-N6-(tert-butoxycarbonyl)-L-lysine

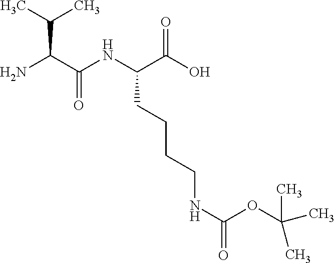 [SMILES: CC(C)[C@H](N)C(=O)N[C@@H](CCCCNC(=O)OC(C)(C)C)C(=O)O]

600.0 mg (1.58 mmol) of N2-[(benzyloxy)carbonyl]-N6-(tert-butoxycarbonyl)-L-lysine were suspended in 25.0 ml of water/ethanol/THF (1:1:0.5), palladium on carbon (10%) was added and the mixture was hydrogenated at RT with hydrogen under standard pressure for 5 h. The catalyst was filtered off and the solvents were evaporated under reduced pressure. The compound obtained was used in the next step without further purification.

LC-MS (Method 1): Rₜ=0.42 min; MS (ESIpos): m/z=247 [M+H]⁺.

180 mg (0.73 mmol) of N6-(tert-butoxycarbonyl)-L-lysine were dissolved in 5.0 ml of DMF, and 74.0 mg (0.73 mmol) of triethylamine were added. 254.6 mg (0.73 mmol) of 2,5-dioxopyrrolidin-1-yl N-[(benzyloxy)carbonyl]-L-valinate and 74.0 mg (0.73 mmol) of triethylamine were then added. The reaction mixture was stirred at RT for 3.5 h. The reaction solution was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 294.1 mg (76% of theory) of N-[(benzyloxy)carbonyl]-L-valyl-N6-(tert-butoxycarbonyl)-L-lysine.

LC-MS (Method 1): Rₜ=0.97 min; MS (ESIpos): m/z=480 [M+H]⁺.

272.2 mg (0.57 mmol) of N-[(benzyloxy)carbonyl]-L-valyl-N6-(tert-butoxycarbonyl)-L-lysine were initially charged in 20.0 ml of ethyl acetate/ethanol/THF (1:1:1), and 27.2 mg of palladium on activated carbon were added. The mixture was hydrogenated with hydrogen at RT under standard pressure for 5 h. The mixture was filtered off with the aid of Celite® and the filter cake was washed with ethyl acetate/ethanol/THF (1:1:1). The solvents were evaporated under reduced pressure and the residue was dried under high vacuum. The title compound (182 mg, 72% of theory) was used in the next reaction step without further purification.

LC-MS (Method 1): Rₜ=0.53 min; MS (ESIpos): m/z=346 [M+H]⁺.

### Intermediate L27

### N-[31-(2,5-Dioxo-2,5-dihydro-1H-pyrrol-1-yl)-29-oxo-4,7,10,13,16,19,22,25-octaoxa-28-azahentriacontan-1-oyl]-L-valyl-N6-(tert-butoxycarbonyl)-L-lysine

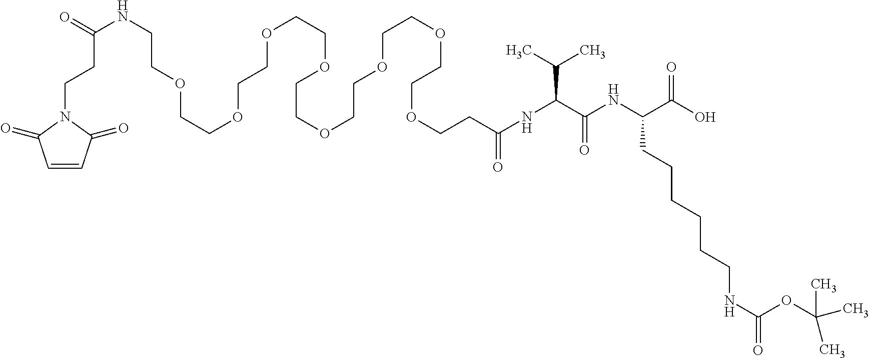 [SMILES: CC(C)[C@H](NC(=O)CCOCCOCCOCCOCCOCCOCCOCCOCCNC(=O)CCN1C(=O)C=CC1=O)C(=O)N[C@@H](CCCCCCNC(=O)OC(C)(C)C)C(=O)O]

30 mg (0.07 mmol) of L-valyl-N6-(tert-butoxycarbonyl)-L-lysine (Intermediate L26) and 46.1 mg (0.07 mmol) of 3-(2,5-dioxo-2,5-dihydro-1H-pyrrol-1-yl)-N-{27-[(2,5-dioxopyrrolidin-1-yl)oxy]-27-oxo-3,6,9,12,15,18,21,24-octaoxaheptacos-1-yl}propanamide were initially charged in 1.5 ml of DMF, and 6.8 mg (0.07 mmol) of 4-methylmorpholine were added. The reaction solution was stirred at RT overnight. The reaction mixture was purified directly by preparative RP-HPLC (column: Reprosil 250×30; 10μ, flow rate: 50 ml/min, MeCN/water). The solvents were evaporated under reduced pressure and the residue was dried under high vacuum. This gave 55.6 mg (90% of theory) of the title compound.

LC-MS (Method 1): Rₜ=0.77 min; MS (ESIpos): m/z=920 [M+H]⁺.

### Intermediate L28

### tert-Butyl 3-formyl-4-({[2-(trimethylsilyl)ethoxy]carbonyl}amino)pyrrolidine-1-carboxylate

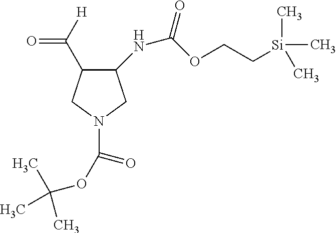 [SMILES: CC(C)(C)OC(=O)N1CC(C=O)C(NC(=O)OCC[Si](C)(C)C)C1]

461.7 mg (1.15 mmol) of 1-tert-butyl 3-ethyl-4-({[2-(trimethylsilyl)ethoxy]carbonyl}amino)pyrrolidine-1,3-dicarboxylate (this compound was prepared according to the literature procedure of WO 2006/066896) were initially charged in 5.0 ml of absolute dichloromethane and the mixture was cooled to −78° C. 326.2 mg (2.29 mmol) of diisobutylaluminium hydride solution (1 M in THF) were then slowly added dropwise and the mixture was stirred at −78° C. for 2 h (monitored by thin-layer chromatography (petroleum ether/ethyl acetate=3:1). 1.3 g (4.59 mmol) of potassium sodium tartrate dissolved in 60 ml of water were added dropwise and the reaction mixture was allowed to warm to RT. Ethyl acetate was added to the reaction mixture and the aqueous phase was extracted three times with ethyl acetate. The combined organic phases were washed once with sat. NaCl solution and dried over magnesium sulphate. The solvent was evaporated under reduced pressure and the residue was dried under high vacuum. This gave 629.0 mg of the title compound as a crude product which was used immediately without further purification in the next reaction step.

### Intermediate L29

### tert-Butyl 3-formyl-4-[({[2-(trimethylsilyl)ethoxy]carbonyl}amino)methyl]pyrrolidine-1-carboxylate

### Mixture of Diastereomers.

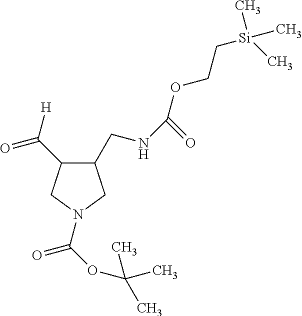 [SMILES: CC(C)(C)OC(=O)N1CC(C=O)C(CNC(=O)OCC[Si](C)(C)C)C1]

807.1 mg (2.34 mmol) of tert-butyl 3-({[tert-butyl(dimethyl)silyl]oxy}methyl)-4-(hydroxymethyl)pyrrolidine-1-carboxylate (prepared according to the literature procedure of WO 2006/100036) were initially charged in 8.0 ml of dichloromethane, and 236.4 mg (2.34 mmol) of triethylamine were added. At 0° C., 267.6 mg (2.34 mmol) of methanesulphonyl chloride were added dropwise, and the reaction mixture stirred at RT overnight. A further 133.8 mg (1.17 mmol) of methanesulphonyl chloride and 118.2 mg (1.17 mmol) of triethylamine were added. The reaction mixture was stirred at RT overnight. The mixture was diluted with dichloromethane and the organic phase was washed in each case once with saturated sodium bicarbonate solution, 5% strength potassium hydrogen sulphate solution and saturated NaCl solution. After drying over magnesium sulphate, the solvent was evaporated under reduced pressure and the residue was purified on Biotage Isolera (silica gel, column 50 g SNAP, flow rate 66 ml/min, cyclohexane/ethyl acetate). The solvents were evaporated under reduced pressure and the residue was dried under high vacuum. This gave 402.0 mg (41% of theory) of the compound tert-butyl 3-({[tert-butyl(dimethyl)silyl]oxy}methyl)-4-{[(methylsulphonyl)oxy]methyl}pyrrolidine-1-carboxylate.

LC-MS (Method 1): Rₜ=1.38 min; MS (ESIpos): m/z=424 [M+H]⁺.

400.0 mg (0.94 mmol) of tert-butyl 3-({[tert-butyl(dimethyl)silyl]oxy}methyl)-4-{[(methylsulphonyl)oxy]methyl}pyrrolidine-1-carboxylate were initially charged in 5.0 ml of DMF, and 98.2 mg (1.51 mmol) of sodium azide were added. The reaction mixture was stirred at 40° C. for 10 h. Another 30.7 mg (0.47 mmol) of sodium azide were then added, and the mixture was stirred at 40° C. for a further 10 h. Ethyl acetate was added and the organic phase was washed repeatedly with water. After drying of the organic phase over magnesium sulphate, the solvent was evaporated under reduced pressure and the residue was dried under high vacuum. This gave 309.5 mg (89% of theory) of the compound tert-butyl 3-(azidomethyl)-4-({[tert-butyl(dimethyl)silyl]oxy}methyl)pyrrolidine-1-carboxylate. The compound was used without further purification in the next step of the synthesis.

LC-MS (Method 1): Rₜ=1.50 min; MS (ESIpos): m/z=371 [M+H]⁺.

250 mg (0.68 mmol) of tert-butyl 3-(azidomethyl)-4-({[tert-butyl(dimethyl)silyl]oxy}methyl)-pyrrolidine-1-carboxylate were dissolved in 10.0 ml of ethyl acetate/ethanol (1:1), and 25.0 mg of palladium on activated carbon (10%) were added. The mixture was hydrogenated with hydrogen at RT under standard pressure for 8 h. The reaction was filtered through Celite® and the filter cake was washed thoroughly with ethyl acetate. The solvent was evaporated under reduced pressure and the residue was dried under high vacuum. This gave 226.2 mg (82% of theory) of the compound tert-butyl 3-(aminomethyl)-4-({[tert-butyl(dimethyl)silyl]oxy}methyl)pyrrolidine-1-carboxylate. The compound was used without further purification in the next step of the synthesis.

LC-MS (Method 1): Rₜ=0.89 min; MS (ESIpos): m/z=345 [M+H]⁺.

715.0 mg (2.08 mmol) of tert-butyl 3-(aminomethyl)-4-({[tert-butyl(dimethyl)silyl]oxy}methyl)-pyrrolidine-Ecarboxylate were dissolved in 15.0 ml of THF, and 2.28 ml (2.28 mmol) of TBAF solution (1M in THF) were added. The reaction mixture was stirred at RT overnight. The solvent was evaporated under reduced pressure and the residue (1.54 g) used without further purification in the next step of the synthesis.

LC-MS (Method 1): Rₜ=0.41 min; MS (ESIpos): m/z=231 [M+H]⁺.

1.54 g (4.88 mmol) of tert-butyl 3-(aminomethyl)-4-(hydroxymethyl)pyrrolidine-Ecarboxylate were initially charged in 1,4-dioxane, and 541.8 mg (4.88 mmol) of calcium chloride (anhydrous) and 488.6 mg (4.88 mmol) of calcium carbonate were added and the mixture was stirred vigorously. 592.8 mg (5.86 mmol) of triethylamine and 1.52 g (5.86 mmol) of 1-({[2-(trimethylsilyl)ethoxy]carbonyl}oxy)pyrrolidine-2,5-dione were then added and the reaction mixture stirred at RT overnight. 644.9 mg (10.7 mmol) of HOAc and ethyl acetate were added. The organic phase was washed twice with water and once with saturated NaCl solution. After drying over magnesium sulphate, the solvent was evaporated under reduced pressure and the residue was purified on silica gel (mobile phase: dichloromethane/methanol=100:1). The solvents were evaporated under reduced pressure and the residue was dried under high vacuum. This gave 346.9 mg (19% of theory) of the compound tert-butyl 3-(hydroxymethyl)-4-[({[2-(trimethylsilyl)ethoxy]carbonyl}amino)methyl]pyrrolidine-1-carboxylate.

LC-MS (Method 1): Rₜ=1.08 min; MS (ESIpos): m/z=375 [M+H]⁺.

804.0 mg (2.15 mmol) of tert-butyl 3-(hydroxymethyl)-4-[({[2-(trimethylsilyl)ethoxy]carbonyl}-amino)methyl]pyrrolidine-1-carboxylate were initially charged in 20.0 ml of chloroform and 20.0 ml of 0.05 N potassium carbonate/0.05 N sodium bicarbonate solution (1:1). 59.7 mg (0.22 mmol) of tetra-n-butylammonium chloride, 429.9 mg (3.22 mmol) of N-chlorosuccinimide and 33.5 mg (0.22 mmol) of TEMPO were then added and the reaction mixture was stirred vigorously at RT overnight. The organic phase was separated off and freed from the solvent under reduced pressure. The residue was purified by silica gel chromatography (mobile phase: cyclohexane/ethyl acetate=3:1). This gave 517.0 mg (46% of theory) of the title compound.

LC-MS (Method 1): Rₜ=1.13 min; MS (ESIpos): m/z=373 [M+H]⁺.

### Intermediate L30

### tert-Butyl 3-({[tert-butyl(dimethyl)silyl]oxy}methyl)-4-formylpyrrolidine-1-carboxylate

### Mixture of Stereoisomers

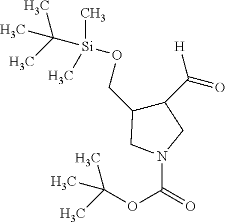 [SMILES: CC(C)(C)OC(=O)N1CC(C=O)C(CO[Si](C)(C)C(C)(C)C)C1]

250.0 mg (0.72 mmol) of tert-butyl 3-({[tert-butyl(dimethyl)silyl]oxy}methyl)-4-(hydroxymethyl)pyrrolidine-1-carboxylate (the compound was prepared according to the literature procedure of WO2006/100036) were initially charged in 12.5 ml of dichloromethane/DMSO (4:1), and 219.6 mg (2.17 mmol) of triethylamine were added. At 2° C., 345.5 mg (2.17 mmol) of sulphur trioxide-pyridine complex were added a little at a time and the mixture was stirred at 2° C. for 3 h. Another 345.5 mg (2.17 mmol) of sulphur trioxide-pyridine complex were added a little at a time and the mixture was stirred at RT for 17 h. The reaction mixture was partitioned between dichloromethane and water. The aqueous phase was extracted three times with dichloromethane and the combined organic phases were washed once with water and dried over magnesium sulphate. The solvent was evaporated under reduced pressure and the residue was dried under high vacuum. The residue was used without further purification in the next step of the synthesis (thin-layer chromatography: petroleum ether/ethyl acetate 7:3).

### Intermediate L31

### Di-tert-butyl {[(tert-butoxycarbonylamino]methyl}malonate

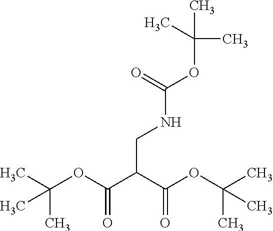 [SMILES: CC(C)(C)OC(=O)NCC(C(=O)OC(C)(C)C)C(=O)OC(C)(C)C]

57.2 g (488.27 mmol) of tert-butyl carbamate, 51.2 ml (683.57 mmol) of a 37% strength solution of formaldehyde in water and 25.9 g (244.13 mmol) of sodium carbonate were added to 600 ml of water. The mixture was warmed until a solution was formed and then stirred at RT for 16 h. The suspension formed was extracted with 500 ml of dichloromethane and the organic phase was separated off, washed with saturated sodium chloride solution and dried over sodium sulphate. The mixture was concentrated on a rotary evaporator and the residue was dried under high vacuum, giving a crystalline solid. The residue was taken up in 1000 ml of absolute THF, and a mixture of 322 ml (3.414 mol) of acetic anhydride and 138 ml (1.707 mol) of pyridine was added dropwise at RT. The reaction mixture was stirred at RT for 16 h and then concentrated on a rotary evaporator, with the water bath at room temperature. The residue was taken up in diethyl ether and washed three times with a saturated sodium bicarbonate solution and once with a saturated sodium chloride solution. The organic phase was dried over sodium sulphate and concentrated on a rotary evaporator and the residue was dried under high vacuum for 2 d. The residue was taken up in 2000 ml of absolute THF, and 456 ml (456.52 mmol) of a 1 M solution of potassium tert-butoxide in THF were added with ice cooling. The mixture was stirred at 0° C. for 20 min, and 100.8 g (456.52 mmol) of di-tert-butyl malonate dissolved in 200 ml of absolute THF were then added dropwise. The mixture was stirred at RT for 48 h, and water was then added. The reaction mixture was concentrated on a rotary evaporator and taken up in 500 ml of ethyl acetate. The mixture was washed with 500 ml of water and 100 ml of a saturated sodium chloride solution and the organic phase was dried over sodium sulphate. The organic phase was concentrated on a rotary evaporator and the residue was dried under high vacuum. The residue was purified by filtration on silica gel (mobile phase: cyclohexane/ethyl acetate, gradient=30:1→5:1). This gave 37.07 g (22% of theory) of the target compound.

LC-MS (Method 6): Rₜ=2.87 min; MS (ESIpos): m/z=346 [M+H]⁺.

### Intermediate L32

### tert-Butyl [3-hydroxy-2-(hydroxymethyl)propyl]carbamate

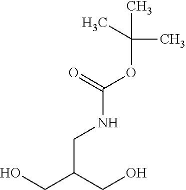

37.0 g (107.11 mmol) of di-tert-butyl (acetoxymethyl)malonate were dissolved in 1000 ml of absolute THF, and 535.5 ml (1071.10 mmol) of a 2 M solution of lithium borohydride in THF were added dropwise with ice cooling. 19.3 ml (1071.10 mmol) of water were added dropwise and the mixture was stirred at RT for 4.5 h. The reaction mixture was concentrated on a rotary evaporator and dried under high vacuum. The residue was taken up in 1500 ml of ethyl acetate, 100 ml of water were added and the mixture was stirred with water cooling (slightly exothermic) for 30 min. The organic phase was separated off and the aqueous phase was extracted twice with 500 ml of ethyl acetate. The organic phase was concentrated on a rotary evaporator and the residue was dried under high vacuum. This gave 20.7 g (94% of theory) of the target compound.

LC-MS (Method 6): Rₜ=1.49 min; MS (EIpos): m/z=106 [M-C₅H₈O₂]⁺.

### Intermediate L33

### tert-Butyl [3-{[tert-butyl(dimethyl)silyl]oxy}-2-(hydroxymethyl)propyl]carbamate

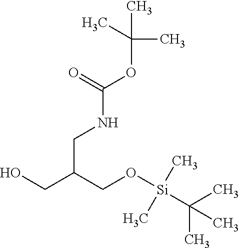 [SMILES: CC(C)(C)OC(=O)NCC(CO)CO[Si](C)(C)C(C)(C)C]

20.00 g (97.44 mmol) of tert-butyl [3-hydroxy-2-(hydroxymethyl)propyl]carbamate were dissolved in 1000 ml of absolute dichloromethane, and 6.63 g (97.44 mmol) of imidazole and 16.16 g (107.18 mmol) of tert-butyl(chloro)dimethylsilane were added at RT. The reaction mixture was stirred at RT for 16 h and washed with semiconcentrated sodium chloride solution. The aqueous phase was extracted with ethyl acetate and the combined organic phases were dried over sodium sulphate, concentrated on a rotary evaporator and dried under high vacuum. This gave 28.50 g (92% of theory) of the target compound.

¹H-NMR (400 MHz, DMSO-dg): δ [ppm]=0.02 (s, 6H), 0.86 (s, 9H), 1.37 (s, 9H), 1.58-1.73 (m, 1H), 2.91 (q, 2H), 3.33-3.36 [m, (2H, hidden)], 3.53-3.58 (m, 2H), 6.65-6.72 (m, 1H).

### Intermediate L34

### tert-Butyl (3-{[tert-butyl(dimethyl)silyl]oxy}-2-formylpropyl)carbamate

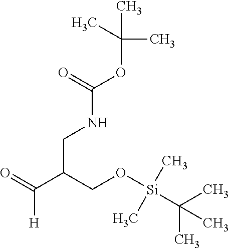 [SMILES: CC(C)(C)OC(=O)NCC(C=O)CO[Si](C)(C)C(C)(C)C]

12.65 g (39.591 mmol) of tert-butyl [3-{[tert-butyl(dimethyl)silyl]oxy}-2-(hydroxy-methyl)propyl]carbamate were dissolved in 200 ml of dichloromethane, and 19.31 g (45.53 mmol) of Dess-Martin periodinane dissolved in 150 ml of dichloromethane were added dropwise at RT. The mixture was stirred at room temperature for 2 h, 250 ml of a semiconcentrated sodium bicarbonate solution and 250 ml of a 10% strength sodium thiosulphate solution were then added and the mixture was stirred for 20 min. The organic phase was separated off and the aqueous phase was extracted with ethyl acetate. The combined organic phases were washed with 300 ml of water, dried over sodium sulphate, concentrated on a rotary evaporator and dried under high vacuum. This gave 11.35 g (90% of theory) of the target compound.

¹H-NMR (400 MHz, DMSO-d₆): δ [ppm]=0.02 (s, 6H), 0.84 (s, 9H), 1.36 (s, 9H), 1.48-1.51 (m, 1H), 3.08-3.32 [m, (1H, hidden)], 3.50-3.58 (m, 2H), 3.81-3.91 (m, 1H), 6.71 (t, 1H), 9.60 (d, 1H).

### Intermediate L35

### tert-Butyl (3-oxopropyl)carbamate

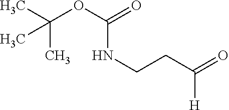

The title compound was prepared according to a method known from the literature (e.g. Jean Bastide et al. J. Med. Chem. 2003, 46(16), 3536-3545).

### Intermediate L36

### N-[(Benzyloxy)carbonyl]-L-valyl-N5-carbamoyl-L-ornithine

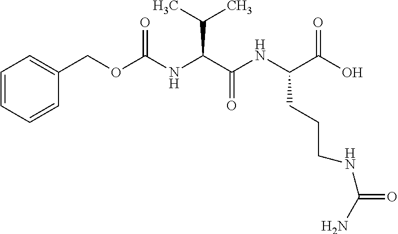 [SMILES: CC(C)[C@H](NC(=O)OCc1ccccc1)C(=O)N[C@@H](CCCNC(N)=O)C(=O)O]

100 mg (0.57 mmol) of N5-carbamoyl-L-ornithine were taken up in 4.0 ml of DMF, and 0.08 ml (0.57 mmol) of triethylamine was added. 199.0 mg (0.57 mmol) of 2,5-dioxopyrrolidin-1-yl-N-[(benzyloxy)carbonyl]-L-valine and 0.08 ml (0.57 mmol) of triethylamine were then added. The mixture was stirred at RT for 48 h. The reaction mixture was purified directly by preparative RP-HPLC (column: Reprosil 250×30; 10μ, flow rate: 50 ml/min, MeCN/water with 0.1% TFA). The solvents were evaporated under reduced pressure and the residue was dried under high vacuum. This gave 75.7 mg (33% of theory) of the title compound.

LC-MS (Method 1): Rₜ=0.69 min; MS (ESIpos): m/z=409 [M+H]⁺.

### Intermediate L37

### L-Valyl-N5-carbamoyl-L-ornithine

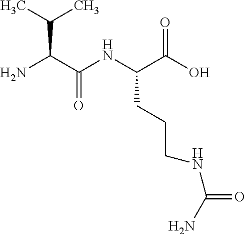 [SMILES: CC(C)[C@H](N)C(=O)N[C@@H](CCCNC(N)=O)C(=O)O]

75.7 mg (0.19 mmol) of Intermediate L36 were suspended in 25 ml of water/ethanol/THF, and 7.5 mg of palladium on activated carbon (10%) were added and the mixture was hydrogenated at RT with hydrogen under standard pressure for 4.5 h. The catalyst was filtered off and the reaction mixture was freed from the solvent under reduced pressure and dried under high vacuum. The residue was used for the next step without further purification. This gave 64.9 mg (93% of theory) of the title compound.

LC-MS (Method 6): Rₜ=0.25 min; MS (ESIpos): m/z=275 [M+H]⁺.

### Intermediate L38

### N-[31-(2,5-Dioxo-2,5-dihydro-1H-pyrrol-1-yl)-29-oxo-4,7,10,13,16,19,22,25-octaoxa-28-azahentriacontan-1-oyl]-L-valyl-N5-carbamoyl-L-ornithine

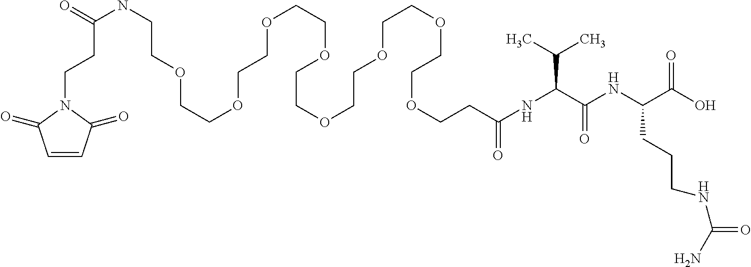 [SMILES: CC(C)[C@H](NC(=O)CCOCCOCCOCCOCCOCCOCCOCCOCCNC(=O)CCN1C(=O)C=CC1=O)C(=O)N[C@@H](CCCNC(N)=O)C(=O)O]

38.3 mg (0.14 mmol) of Intermediate L37 were initially charged in 3.0 ml of DMF, and 96.4 mg (0.14 mmol) of 3-(2,5-dioxo-2,5-dihydro-1H-pyrrol-1-yl)-N-{27-[(2,5-dioxopyrrolidin-1-yl)oxy]-27-oxo-3,6,9,12,15,18,21,24-octaoxaheptacos-1-yl}propanamide and 39.0 μl (0.28 mmol) of triethylamine were added. The mixture was stirred at RT overnight. 16.0 μl (0.28 mmol) of HOAc were then added, and the reaction mixture was purified directly by preparative RP-HPLC (column: Reprosil 250×30; 10μ, flow rate: 50 ml/min, MeCN/water). The solvents were evaporated under reduced pressure and the residue was dried under high vacuum. This gave 58.9 mg (45% of theory) of the title compound.

LC-MS (Method 1): Rₜ=0.61 min; MS (ESIpos): m/z=849 [M+H]⁺.

### Intermediate L39

### 2-(Trimethylsilyl)ethyl (2-sulphanylethyl)carbamate

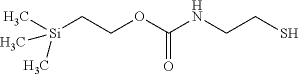

300 mg (2.64 mmol) of 2-aminoethanethiol hydrochloride (1:1) were initially charged in 3.0 ml of dichloromethane, and 668.0 mg (6.60 mmol) of triethylamine and 719.1 mg (2.77 mmol) of 1-({[2-(trimethylsilyl)ethoxy]carbonyl}oxy)pyrrolidine-2,5-dione were added. The mixture was stirred at RT for 2 days (monitored by thin-layer chromatography: dichloromethane/methanol=100:1.5). Ethyl acetate was added and the reaction mixture was washed three times with water. The organic phase was washed twice with saturated NaCl solution and dried over magnesium sulphate. The solvent was evaporated under reduced pressure and the residue was dried under high vacuum. The compound was used without further purification in the next step of the synthesis.

### Intermediate L40

### N-[31-(2,5-Dioxo-2,5-dihydro-1H-pyrrol-1-yl)-29-oxo-4,7,10,13,16,19,22,25-octaoxa-28-azahentriacontan-1-oyl]-L-valyl-N6-(tert-butoxycarbonyl)-L-lysine

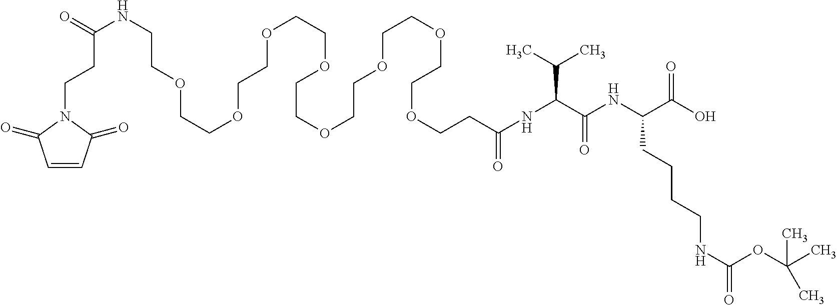 [SMILES: CC(C)[C@H](NC(=O)CCOCCOCCOCCOCCOCCOCCOCCOCCNC(=O)CCN1C(=O)C=CC1=O)C(=O)N[C@@H](CCCCNC(=O)OC(C)(C)C)C(=O)O]

600 mg (1.58 mmol) of N2-[(benzyloxy)carbonyl]-N6-(tert-butoxycarbonyl)-L-lysine were hydrogenated in 25.0 ml of water/ethanol/THF (1:1:0.5) using palladium on carbon (10%) at RT under standard pressure with hydrogen. The compound N6-(tert-butoxycarbonyl)-L-lysine is used without further purification in the next step of the synthesis.

LC-MS (Method 1): Rₜ=0.99 min; MS (ESIpos): m/z=247 [M+H]⁺.

180.0 mg (0.73 mmol) of N6-(tert-butoxycarbonyl)-L-lysine were dissolved in 5.0 ml of DMF, and 74.0 mg (0.73 mmol) of triethylamine were added. 254.6 mg (0.73 mmol) of 2,5-dioxopyrrolidin-1-yl N-[(benzyloxy)carbonyl]-L-valinate and 74.0 mg (0.73 mmol) of triethylamine were added. The reaction mixture was stirred at RT for 3.5 h. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 294.1 mg (76% of theory) of the compound N-[(benzyloxy)carbonyl]-L-valyl-N6-(tert-butoxycarbonyl)-L-lysine.

LC-MS (Method 1): Rₜ=0.97 min; MS (ESIpos): m/z=480 [M+H]⁺.

272.2 mg (0.57 mmol) of N-[(benzyloxy)carbonyl]-L-valyl-N6-(tert-butoxycarbonyl)-L-lysine were dissolved in 20 ml of ethyl acetate/ethanol/THF (1:1:1), 27.2 mg of palladium on activated carbon were added and the mixture was hydrogenated under standard pressure and at RT with hydrogen. The mixture was filtered through Celite® and the filter cake was washed thoroughly with ethyl acetate/ethanol/THF (1:1:1). The solvents were evaporated under reduced pressure and the residue was dried under high vacuum. This gave 182.0 mg (72% of theory) of the compound L-valyl-N6-(tert-butoxycarbonyl)-L-lysine.

LC-MS (Method 1): Rₜ=0.53 min; MS (ESIpos): m/z=346 [M+H]⁺.

30.0 mg (0.07 mmol) of L-valyl-N6-(tert-butoxycarbonyl)-L-lysine and 46.1 mg (0.07 mmol) of 3-(2,5-dioxo-2,5-dihydro-1H-pyrrol-1-yl)-N-{27-[(2,5-dioxopyrrolidin-1-yl)oxy]-27-oxo-3,6,9,12,15,18,21,24-octaoxaheptacos-1-yl}propanamide were dissolved in 1.5 ml of DMF, and 6.8 mg (0.07 mmol) of 4-methylmorpholine were added. The reaction mixture was stirred at RT overnight. The reaction mixture was purified directly by preparative RP-HPLC (column: Reprosil 250×30; 10μ, flow rate: 50 ml/min, MeCN/water). The solvents were evaporated under reduced pressure and the residue was dried under high vacuum. This gave 55.6 mg (90% of theory) of the title compound.

LC-MS (Method 1): Rₜ=0.77 min; MS (ESIpos): m/z=920 [M+H]⁺.

### Intermediate L41

### N-[19-(2,5-Dioxo-2,5-dihydro-1H-pyrrol-1-yl)-17-oxo-4,7,10,13-tetraoxa-16-azanonadecan-1-oyl]-L-valyl-N6-(tert-butoxycarbonyl)-L-lysine

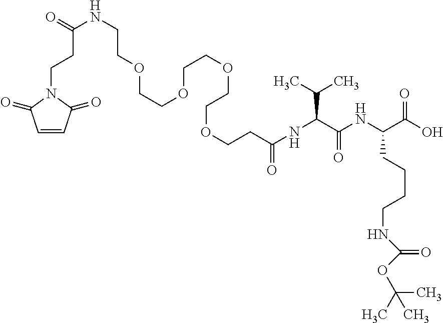 [SMILES: CC(C)[C@H](NC(=O)CCOCCOCCOCCOCCNC(=O)CCN1C(=O)C=CC1=O)C(=O)N[C@@H](CCCCNC(=O)OC(C)(C)C)C(=O)O]

600 mg (1.58 mmol) of N2-[(benzyloxy)carbonyl]-N6-(tert-butoxycarbonyl)-L-lysine were hydrogenated in 25.0 ml of water/ethanol/THF (1:1:0.5) using palladium on carbon (10%) at RT under standard pressure with hydrogen. The compound N6-(tert-butoxycarbonyl)-L-lysine is used without further purification in the next step of the synthesis.

LC-MS (Method 1): Rₜ=0.99 min; MS (ESIpos): m/z=247 [M+H]⁺.

180.0 mg (0.73 mmol) of N6-(tert-butoxycarbonyl)-L-lysine were dissolved in 5.0 ml of DMF, and 74.0 mg (0.73 mmol) of triethylamine were added. 254.6 mg (0.73 mmol) of 2,5-dioxopyrrolidin-1-yl N-[(benzyloxy)carbonyl]-L-valinate and 74.0 mg (0.73 mmol) of triethylamine were added. The reaction mixture was stirred at RT for 3.5 h. The reaction mixture was purified directly by preparative RP-HPLC (column: Reprosil 250×30; 10μ, flow rate: 50 ml/min, MeCN/water, 0.1% TFA). The solvents were then evaporated under reduced pressure and the residue was dried under high vacuum. This gave 294.1 mg (76% of theory) of the compound N-[(benzyloxy)carbonyl]-L-valyl-N6-(tert-butoxycarbonyl)-L-lysine.

LC-MS (Method 1): Rₜ=0.97 min; MS (ESIpos): m/z=480 [M+H]⁺.

272.2 mg (0.57 mmol) of N-[(benzyloxy)carbonyl]-L-valyl-N6-(tert-butoxycarbonyl)-L-lysine were dissolved in 20.0 ml of ethyl acetate/ethanol/THF (1:1:1), 27.2 mg of palladium on activated carbon were added and the mixture was hydrogenated under standard pressure and at RT with hydrogen. The mixture was filtered through Celite® and the filter cake was washed thoroughly with ethyl acetate/ethanol/THF (1:1:1). The solvents were evaporated under reduced pressure and the residue was dried under high vacuum. This gave 182.0 mg (72% of theory) of the compound L-valyl-N6-(tert-butoxycarbonyl)-L-lysine.

LC-MS (Method 1): Rₜ=0.53 min; MS (ESIpos): m/z=346 [M+H]⁺.

30.0 mg (0.07 mmol) of L-valyl-N6-(tert-butoxycarbonyl)-L-lysine and 34.3 mg (0.07 mmol) of 3-(2,5-dioxo-2,5-dihydro-1H-pyrrol-1-yl)-N-{15-[(2,5-dioxopyrrolidin-1-yl)oxy]-15-oxo-3,6,9,12-tetraoxapentadec-1-yl}propanamide were dissolved in 1.5 ml of DMF, and 6.8 mg (0.07 mmol) of 4-methylmorpholine were added. The reaction mixture was stirred at RT overnight. The reaction mixture was purified directly by preparative RP-HPLC (column: Reprosil 250×30; 10μ, flow rate: 50 ml/min, MeCN/water). The solvents were evaporated under reduced pressure and the residue was dried under high vacuum. This gave 40.6 mg (82% of theory) of the title compound.

LC-MS (Method 1): Rₜ=0.73 min; MS (ESIpos): m/z=744 [M+H]⁺.

### Intermediate L42

### N-[19-(2,5-Dioxo-2,5-dihydro-1H-pyrrol-1-yl)-17-oxo-4,7,10,13-tetraoxa-16-azanonadecan-1-oyl]-L-valyl-N5-carbamoyl-L-ornithine

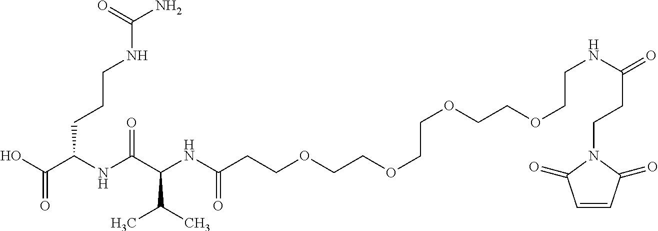 [SMILES: CC(C)[C@H](NC(=O)CCOCCOCCOCCOCCNC(=O)CCN1C(=O)C=CC1=O)C(=O)N[C@@H](CCCNC(N)=O)C(=O)O]

50.0 mg (0.18 mmol) of L-valyl-N5-carbamoyl-L-ornithine (Intermediate L37) were initially charged in DMF, and 93.6 mg (0.18 mmol) of 3-(2,5-dioxo-2,5-dihydro-1H-pyrrol-1-yl)-N-{15-[(2,5-dioxopyrrolidin-1-yl)oxy]-15-oxo-3,6,9,12-tetraoxapentadec-1-yl}propanamide and 36.9 mg (0.37 mmol) of triethylamine were added. The reaction mixture was stirred at RT overnight. 21.9 mg (0.37 mmol) of HOAc were added and the reaction mixture was purified directly by preparative RP-HPLC (column: Reprosil 250×30; 10μ, flow rate: 50 ml/min, MeCN/water). The solvents were evaporated under reduced pressure and the residue was dried under high vacuum. This gave 20.6 mg (14% of theory) of the title compound.

LC-MS (Method 1): Rₜ=0.55 min; MS (ESIpos): m/z=673 [M+H]⁺.

### Intermediate L43

### N-[67-(2,5-Dioxo-2,5-dihydro-1H-pyrrol-1-yl)-65-oxo-4,7,10,13,16,19,22,25,28,31,34,37,40,43,46,49,52,55,58,61-icosaoxa-64-azaheptahexacontan-1-oyl]-L-valyl-N5-carbamoyl-L-ornithine

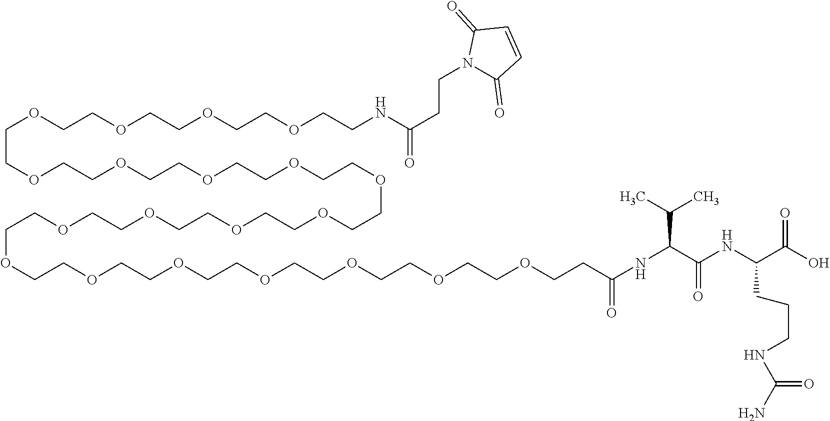 [SMILES: CC(C)[C@H](NC(=O)CCOCCOCCOCCOCCOCCOCCOCCOCCOCCOCCOCCOCCOCCOCCOCCOCCOCCOCCOCCOCCNC(=O)CCN1C(=O)C=CC1=O)C(=O)N[C@@H](CCCNC(N)=O)C(=O)O]

11.3 mg (0.04 mmol) of L-valyl-N5-carbamoyl-L-ornithine (Intermediate L37) were initially charged in DMF, and 50.0 mg (0.04 mmol) of 3-(2,5-dioxo-2,5-dihydro-1H-pyrrol-1-yl)-N-{63-[(2,5-dioxopyrrolidin-1-yl)oxy]-63-oxo-3,6,9,12,15,18,21,24,27,30,33,36,39,42,45,48,51,54,57,60-icosaoxatrihexacont-1-yl}propanamide and 8.3 mg (0.08 mmol) of triethylamine were added. The reaction mixture was stirred at RT overnight. 4.9 mg (0.08 mmol) of HOAc were added and the reaction mixture was purified directly by preparative RP-HPLC (column: Reprosil 250×30; 10μ, flow rate: 50 ml/min, MeCN/water). The solvents were evaporated under reduced pressure and the residue was dried under high vacuum. This gave 15.8 mg (20% of theory) of the title compound.

LC-MS (Method 4): Rₜ=0.94 min; MS (ESIpos): m/z=1377 [M+H]⁺.

### Intermediate L44

### N-[19-(2,5-Dioxo-2,5-dihydro-1H-pyrrol-1-yl)-17-oxo-4,7,10,13-tetraoxa-16-azanonadecan-1-oyl]-L-valyl-L-alanine

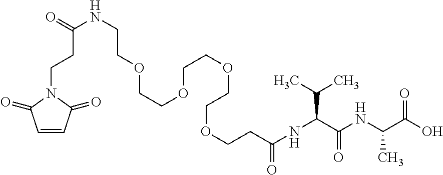 [SMILES: CC(C)[C@H](NC(=O)CCOCCOCCOCCOCCNC(=O)CCN1C(=O)C=CC1=O)C(=O)N[C@@H](C)C(=O)O]

73.3 mg (0.39 mmol) of L-valyl-L-alanine were dissolved in 7.0 ml of DMF, and 200.0 mg (0.39 mmol) of 3-(2,5-dioxo-2,5-dihydro-1H-pyrrol-1-yl)-N-{15-[(2,5-dioxopyrrolidin-1-yl)oxy]-15-oxo-3,6,9,12-tetraoxapentadec-1-yl}propanamide and 78.8 mg (0.78 mmol) of triethylamine were added. The reaction mixture was stirred at RT overnight. The reaction mixture was purified directly by preparative RP-HPLC (column: Reprosil 250×30; 10μ, flow rate: 50 ml/min, MeCN/water). The solvents were evaporated under reduced pressure and the residue was dried under high vacuum. This gave 103.3 mg (45% of theory) of the title compound.

LC-MS (Method 1): Rₜ=0.58 min; MS (ESIpos): m/z=587 [M+H]⁺.

### Intermediate L45

### tert-Butyl (2S)-2-[(tert-butoxycarbonyl)amino]-4-oxobutanoate

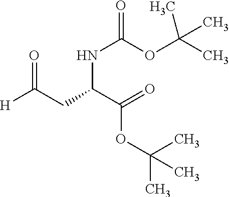 [SMILES: CC(C)(C)OC(=O)N[C@@H](CC=O)C(=O)OC(C)(C)C]

2.00 g (7.26 mmol) of tert-butyl N-(tert-butoxycarbonyl)-L-homoserinate were dissolved in 90 ml of dichloromethane, and 1.76 ml of pyridine and 4.62 g (10.90 mmol) of 1,1,1-triacetoxy-1 lambda⁵,2-benziodoxol-3(1H)-on (Dess-Martin periodinane) were then added. The reaction was stirred at RT for 2 h and then diluted with 200 ml of dichloromethane and extracted twice with 10% strength sodium thiosulphate solution and then successively twice with 5% strength citric acid and twice with saturated sodium bicarbonate solution. The organic phase was separated off, dried over sodium sulphate and then concentrated under reduced pressure. 100 ml of diethyl ether and cyclohexane (v/v=1:1) were added to the residue and the mixture was somewhat concentrated, resulting in the formation of a white precipitate. This was filtered off with suction. The filtrate was concentrated on a rotary evaporator and dried under high vacuum, giving 1.74 g (88% of theory) of the target compound as a light-yellow oil.

LC-MS (Method 1): Rₜ=0.85 min; MS (ESIpos): m/z=274 [M+H]⁺.

¹H-NMR (400 MHz, DMSO-d₆): δ [ppm]=1.38 (s, 18H), 2.64-2.81 (m, 2H), 4.31-4.36 (m, 1H), 7.23 (d, 1H), 9.59 (s, 1H).

### Intermediate L46

### Trifluoroacetic acid/tert-butyl N-[2-(2,5-dioxo-2,5-dihydro-1H-pyrrol-1-yl)ethyl]-L-glutaminate (1:1)

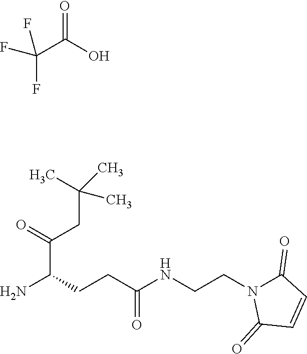 [SMILES: O=C(O)C(F)(F)F ; CC(C)(C)CC(=O)[C@@H](N)CCC(=O)NCCN1C(=O)C=CC1=O]

The title compound was prepared by first coupling 200 mg (0.79 mmol) of trifluoroacetic acid/1-(2-aminoethyl)-1H-pyrrole-2,5-dione (1:1) with 263 mg (0.87 mmol) of (4S)-5-tert-butoxy-4-[(tert-butoxycarbonyl)amino]-5-oxopentanoic acid/trifluoroacetic acid (1:1) in the presence of EDC/HOBT and N,N-diisopropylethylamine and then deprotecting the amino group under gentle conditions by stirring for 1 h in 10% strength trifluoroacetic acid in DCM at RT. Freeze-drying from acetonitrile/water gave 85 mg (20% of theory) of the title compound over 2 steps.

LC-MS (Method 1): Rₜ=0.37 min; MS (ESIpos): m/z=326 [M+H]⁺.

### Intermediate L47

### Trifluoroacetic acid/beta-alanyl-L-alanyl-N5-carbamoyl-N-[4-(2,5-dioxo-2,5-dihydro-1H-pyrrol-1-yl)phenyl]-L-ornithinamide (1:1)

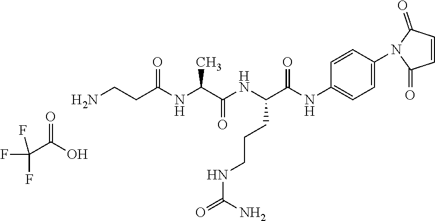 [SMILES: O=C(O)C(F)(F)F ; C[C@H](NC(=O)CCN)C(=O)N[C@@H](CCCNC(N)=O)C(=O)Nc1ccc(N2C(=O)C=CC2=O)cc1]

The title compound was prepared by coupling Intermediate L8 with 2,5-dioxopyrrolidin-1-yl N-(tert-butoxycarbonyl)-beta-alaninate and subsequent deprotection with TFA.

LC-MS (Method 3): Rₜ=1.36 min; MS (ESIpos): m/z=488 (M+H)⁺.

### Intermediate L48

### Trifluoroacetic acid/(1R,2S)-2-amino-N-[2-(2,5-dioxo-2,5-dihydro-1H-pyrrol-1-yl)ethyl]cyclopentanecarboxamide (1:1)

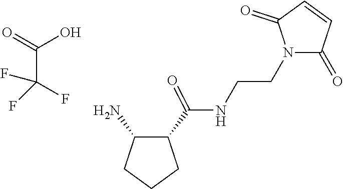 [SMILES: O=C(O)C(F)(F)F ; N[C@H]1CCC[C@H]1C(=O)NCCN1C(=O)C=CC1=O]

The title compound was prepared from commercially available (1R,2S)-2-[(tert-butoxycarbonyl)amino]cyclopentanecarboxylic acid analogously to Intermediate L2.

LC-MS (Method 3): Rₜ=1.22 min; MS (ESIpos): m/z=252 (M+H)⁺.

### Intermediate L49

### Trifluoroacetic acid/tert-butyl N-(bromoacetyl)-L-valyl-L-alanyl-L-lysinate (1:1)

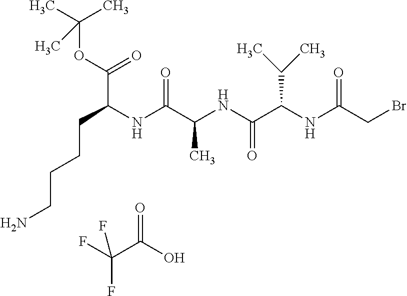 [SMILES: CC(C)[C@H](NC(=O)CBr)C(=O)N[C@@H](C)C(=O)N[C@@H](CCCCN)C(=O)OC(C)(C)C ; O=C(O)C(F)(F)F]

The title compound was prepared by first coupling commercially available bromoacetic anhydride with the partially protected peptide tert-butyl L-valyl-L-alanyl-N⁶-(tert-butoxycarbonyl)-L-lysinate, prepared according to classical methods of peptide chemistry, in the presence of N,N-diisopropyl-ethylamine in dichloromethane. This was followed by deprotection at the amino group under gentle conditions by stirring in 10% strength trifluoroacetic acid in DCM at RT, giving the title compound in 49% yield over 2 steps.

LC-MS (Method 1): Rₜ=1.09 min; MS (ESIpos): m/z=593 and 595 (M+H)⁺.

### Intermediate L50

### Trifluoroacetic acid/(1 S,3R)-3-amino-N-[2-(2,5-dioxo-2,5-dihydro-1H-pyrrol-1-yl)ethyl]cyclopentanecarboxamide (1:1)

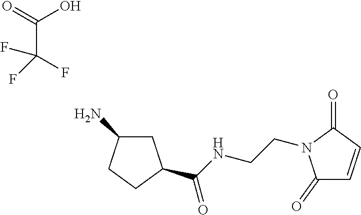 [SMILES: O=C(O)C(F)(F)F ; N[C@@H]1CC[C@H](C(=O)NCCN2C(=O)C=CC2=O)C1]

The title compound was prepared from commercially available (1S,3R)-3-[(tert-butoxycarbonyl)amino]cyclopentanecarboxylic acid and likewise commercially available trifluoroacetic acid/1-(2-aminoethyl)-1H-pyrrole-2,5-dione (1:1) by coupling with HATU in the presence of N,N-diisopropylethylamine and subsequent deprotection with TFA.

HPLC (Method 11): Rₜ=0.2 min;

LC-MS (Method 3): Rₜ=0.88 min; MS (ESIpos): m/z=252 (M+H)⁺.

### Intermediate L51

### Trifluoroacetic acid/(1R,3R)-3-amino-N-[2-(2,5-dioxo-2,5-dihydro-1H-pyrrol-1-yl)ethyl]cyclopentanecarboxamide (1:1)

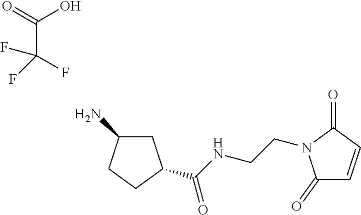 [SMILES: O=C(O)C(F)(F)F ; N[C@@H]1CC[C@@H](C(=O)NCCN2C(=O)C=CC2=O)C1]

The title compound was prepared from commercially available (1R,3R)-3-[(tert-butoxycarbonyl)amino]cyclopentanecarboxylic acid and likewise commercially available trifluoroacetic acid/1-(2-aminoethyl)-1H-pyrrole-2,5-dione (1:1) by coupling with HATU in the presence of N,N-diisopropylethylamine and subsequent deprotection with TFA.

LC-MS (Method 3): Rₜ=0.98 min; MS (ESIpos): m/z=250 (M−H)⁻.

### Intermediate L52

### Trifluoroacetic acid/N-(2-aminoethyl)-2-bromo acetamide (1:1)

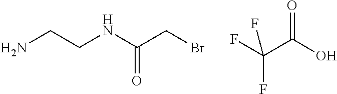

420 mg (2.62 mmol) of tert-butyl (2-aminoethyl)carbamate were taken up in 50 ml of dichloromethane, and 817 mg (3.15 mmol) of bromoacetic anhydride and 913 μl (5.24 mmol) of N,N-diisopropylethylamine were added. The reaction was stirred at RT for 1 h and then concentrated under reduced pressure. The residue was purified by preparative HPLC.

This gave 577 mg of the protected intermediate which were then taken up in 50 ml of dichloromethane, and 10 ml of trifluoroacetic acid were added. After 1 h of stirring at RT, the reaction was concentrated under reduced pressure and the residue was lyophilized from acetonitrile/water. This gave 705 mg (65% of theory) of the title compound.

LC-MS (Method 3): Rₜ=0.34 min; MS (ESIpos): m/z=181 and 183 (M+H)⁺.

### Intermediate L53

### Trifluoroacetic acid/(1S,3S)-3-amino-N-[2-(2,5-dioxo-2,5-dihydro-1H-pyrrol-1-yl)ethyl]cyclopentanecarboxamide (1:1)

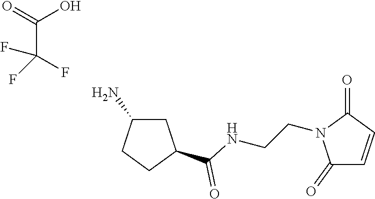 [SMILES: O=C(O)C(F)(F)F ; N[C@H]1CC[C@H](C(=O)NCCN2C(=O)C=CC2=O)C1]

The title compound was prepared from commercially available (1S,3S)-3-[(tert-butoxycarbonyl)amino]cyclopentanecarboxylic acid and likewise commercially available trifluoroacetic acid/l-(2-aminoethyl)-1H-pyrrole-2,5-dione (1:1) by coupling with HATU in the presence of N,N-diisopropylethylamine and subsequent deprotection with TFA.

HPLC (Method 11): Rₜ=0.19 min;

LC-MS (Method 3): Rₜ=0.88 min; MS (ESIpos): m/z=250 (M−H)⁻.

### Intermediate L54

### Trifluoroacetic acid/(1R,3S)-3-amino-N-[2-(2,5-dioxo-2,5-dihydro-1H-pyrrol-1-yl)ethyl]cyclopentanecarboxamide (1:1)

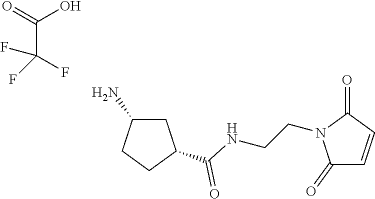 [SMILES: O=C(O)C(F)(F)F ; N[C@H]1CC[C@@H](C(=O)NCCN2C(=O)C=CC2=O)C1]

The title compound was prepared from commercially available (1R,3S)-3-[(tert-butoxycarbonyl)amino]cyclopentanecarboxylic acid and likewise commercially available trifluoroacetic acid/1-(2-aminoethyl)-1H-pyrrole-2,5-dione (1:1) by coupling with HATU in the presence of N,N-diisopropylethylamine and subsequent deprotection with TFA.

LC-MS (Method 3): Rₜ=0.89 min; MS (ESIpos): m/z=252 (M+H)⁺.

### Intermediate L55

### Trifluoroacetic acid/tert-butyl-N6-D-alanyl-N2-{N-[6-(2,5-dioxo-2,5-dihydro-1H-pyrrol-1-yl)-hexanoyl]-L-valyl-L-alanyl}-L-lysinate (1:1)

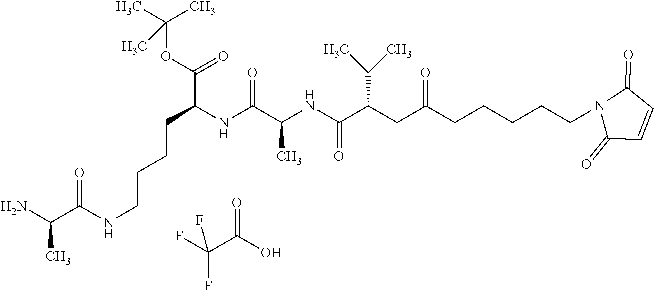 [SMILES: CC(C)[C@H](CC(=O)CCCCCN1C(=O)C=CC1=O)C(=O)N[C@@H](C)C(=O)N[C@@H](CCCCNC(=O)[C@@H](C)N)C(=O)OC(C)(C)C ; O=C(O)C(F)(F)F]

The title compound was prepared by first coupling Intermediate L6 with N-(tert-butoxycarbonyl)-D-alanine in the presence of HATU, followed by deprotection at the amino group under gentle conditions by stirring for 90 minutes in 5% strength trifluoroacetic acid in DCM at RT.

HPLC (Method 11): Rₜ=1.35 min;

LC-MS (Method 1): Rₜ=0.67 min; MS (ESIpos): m/z=637 (M+H)⁺.

### Intermediate L56

### Trifluoroacetic acid/tert-butyl-N-[6-(2,5-dioxo-2,5-dihydro-1H-pyrrol-1-yl)hexanoyl]-L-valyl-L-alanyl-N6-{[(1R,3S)-3-aminocyclopentyl]carbonyl}-L-lysinate (1:1)

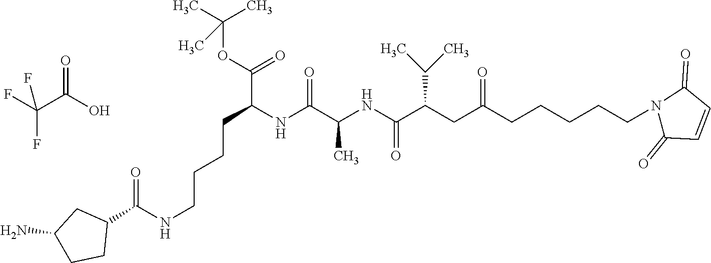

The title compound was prepared by first coupling Intermediate L6 with (1R,3S)-3-[(tert-butoxycarbonyl)amino]cyclopentanecarboxylic acid in the presence of HATU, followed by deprotection at the amino group under gentle conditions by stirring for 15 minutes in 25% strength trifluoroacetic acid in DCM at RT.

HPLC (Method 11): Rₜ=1.4 min;

LC-MS (Method 1): Rₜ=0.7 min; MS (ESIpos): m/z=677 (M+H)⁺.

### Intermediate L57

### Methyl (2S)-4-oxo-2-({[2-(trimethylsilyl)ethoxy]carbonyl}amino)butanoate

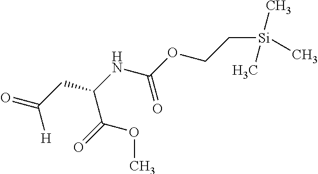 [SMILES: COC(=O)[C@H](CC=O)NC(=O)OCC[Si](C)(C)C]

500.0 mg (2.72 mmol) of methyl L-asparaginate hydrochloride and 706.3 mg (2.72 mmol) of 2-(trimethylsilyl)ethyl 2,5-dioxopyrrolidine-1-carboxylate were initially charged in 5.0 ml of 1,4-dioxane, and 826.8 mg (8.17 mmol) of triethylamine were added. The reaction mixture was stirred at RT overnight. The reaction mixture was purified directly by preparative RP-HPLC (column: Reprosil 250×40; 10μ, flow rate: 50 ml/min, MeCN/water, 0.1% TFA). The solvents were then evaporated under reduced pressure and the residue was dried under high vacuum. This gave 583.9 mg (74% of theory) of the compound (3S)-4-methoxy-4-oxo-3-({[2-(trimethylsilyl)ethoxy]carbonyl}amino)butanoic acid.

LC-MS (Method 1): Rₜ=0.89 min; MS (ESIneg): m/z=290 (M−H)⁻.

592.9 mg of (3S)-4-methoxy-4-oxo-3-({[2-(trimethylsilyl)ethoxy]carbonyl}amino)butanoic acid were initially charged in 10.0 ml of 1,2-dimethoxyethane, the mixture was cooled to −15° C. and 205.8 mg (2.04 mmol) of 4-methylmorpholine and 277.9 mg (2.04 mmol) of isobutyl chloroformate were added. The precipitate was filtered off with suction after 15 min and twice with in each case 10.0 ml of 1,2-dimethoxyethane. The filtrate was cooled to −10° C., and 115.5 mg (3.05 mmol) of sodium borohydride dissolved in 10 ml of water were added with vigorous stirring. The phases were separated and the organic phase was washed in each case once with saturated sodium bicarbonate solution and saturated NaCl solution. The organic phase was dried over magnesium sulphate, the solvent was evaporated under reduced pressure and the residue was dried under high vacuum. This gave 515.9 mg (91% of theory) of the compound methyl N-{[2-(trimethylsilyl)ethoxy]carbonyl}-L-homoserinate.

LC-MS (Method 1): Rₜ=0.87 min; MS (ESIpos): m/z=278 (M+H)⁺.

554.9 mg (2.00 mmol) of methyl N-{[2-(trimethylsilyl)ethoxy]carbonyl}-L-homoserinate were initially charged in 30.0 ml of dichloromethane, and 1.27 g (3.0 mmol) of Dess-Martin periodinane and 474.7 mg (6.00 mmol) of pyridine were added. The mixture was stirred at RT overnight. After 4 h, the reaction was diluted with dichloromethane and the organic phase was washed in each case three times with 10% strength Na₂S₂O₃ solution, 10% strength citric acid solution and saturated sodium bicarbonate solution. The organic phase was dried over magnesium sulphate and the solvent was evaporated under reduced pressure. This gave 565.7 mg (97% of theory) of the title compound.

¹H-NMR (400 MHz, DMSO-dg): δ [ppm]=0.03 (s, 9H), 0.91 (m, 2H), 2.70-2.79 (m, 1H), 2.88 (dd, 1H), 3.63 (s, 3H), 4.04 (m, 2H), 4.55 (m, 1H), 7.54 (d, 1H), 9.60 (t, 1H).

### Intermediate L58

### 2-(Trimethylsilyl)ethyl (3-oxopropyl)carbamate

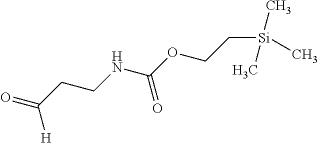

434.4 mg (5.78 mmol) of 3-amino-1-propanol and 1.50 g (5.78 mmol) of 2-(trimethylsilyl)ethyl 2,5-dioxopyrrolidine-1-carboxylate were dissolved in 10.0 ml of dichloromethane, 585.3 mg (5.78 mmol) of triethylamine were added and the mixture was stirred at RT overnight. The reaction mixture was diluted with dichloromethane and the organic phase was washed with water and saturated sodium bicarbonate solution and then dried over magnesium sulphate. The solvent was evaporated under reduced pressure. The residue 2-(trimethylsilyl)ethyl (3-bydroxypropyl)carbamate (996.4 mg, 79% of theory) was dried under high vacuum and used without further purification in the next step of the synthesis.

807.0 mg (3.68 mmol) of 2-(trimethylsilyl)ethyl (3-hydroxypropyl)carbamate were initially charged in 15.0 ml of chloroform and 15.0 ml of 0.05 N potassium carbonate/0.05 N sodium bicarbonate solution (1:1). 102.2 mg (0.37 mmol) of tetra-n-butylammonium chloride, 736.9 mg (5.52 mmol) of N-chlorosuccinimide and 57.5 mg (0.37 mmol) of TEMPO were then added and the reaction mixture was stirred vigorously at RT overnight. The reaction mixture was diluted with dichloromethane and the organic phase was washed with water and saturated NaCl solution. The organic phase was dried over magnesium sulphate and the solvent was evaporated under reduced pressure. The residue was dried under high vacuum and used without further purification in the next step of the synthesis (890.3 mg).

### Intermediate L59

### Trifluoroacetic acid/1-{2-[2-(2-aminoethoxy)ethoxy]ethyl}-1H-pyrrole-2,5-dione (1:1)

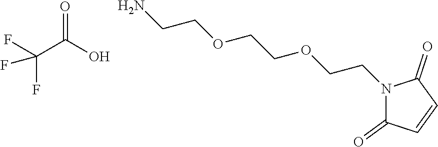 [SMILES: O=C(O)C(F)(F)F ; NCCOCCOCCN1C(=O)C=CC1=O]

300.0 mg (0.91 mmol) of tert-butyl (2-{2-[2-(2,5-dioxo-2,5-dihydro-1H-pyrrol-1-yl)ethoxy]ethoxy}ethyl)carbamate were initially charged in dichloromethane, 4.2 g (36.54 mmol) of TFA were added and the mixture was stirred at RT for 1 h (monitored by TLC: dichloromethane/methanol 10:1). The volatile components were evaporated under reduced pressure and the residue was co-distilled four times with dichloromethane. The residue was dried under high vacuum and used without further purification in the next step of the synthesis.

LC-MS (Method 1): Rₜ=0.19 min; MS (ESIpos): m/z=229 (M+H)⁺.

### Intermediate L60

### 6-(2,5-Dioxo-2,5-dihydro-1H-pyrrol-1-yl)hexanoyl chloride

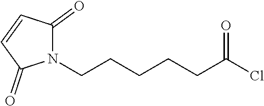 [SMILES: O=C(Cl)CCCCCN1C(=O)C=CC1=O]

200.0 mg (0.95 mmol) of 6-(2,5-dioxo-2,5-dihydro-1H-pyrrol-1-yl)hexanoic acid were dissolved in 4.0 ml of dichloromethane, and 338.0 mg (2.84 mmol) of thionyl chloride were added. The reaction mixture was stirred at RT for 3 h, and 1 drop of DMF was then added. The mixture was stirred for another 1 h. The solvent was evaporated under reduced pressure and the residue was co-distilled three times with dichloromethane. The crude product was used without further purification in the next step of the synthesis.

### Intermediate L61

### Trifluoroacetic acid/2-(trimethylsilyl)ethyl N-[6-(2,5-dioxo-2,5-dihydro-1H-pyrrol-1-yl)hexanoyl]-L-valyl-L-alanyl-L-lysinate (1:1)

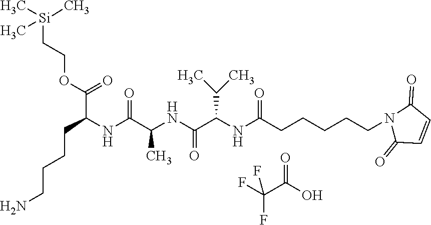

First, the tripeptide derivative 2-(trimethylsilyl)ethyl L-valyl-L-alanyl-N6-(tert-butoxycarbonyl)-L-lysinate was prepared from N2-[(benzyloxy)carbonyl]-N6-(tert-butoxycarbonyl)-L-lysine according to classical methods of peptide chemistry (esterification with 2-(trimethylsilylethanol using EDCI/DMAP, hydrogenolysis, coupling with N-[(benzyloxy)carbonyl]-L-valyl-L-alanine in the presence of HATU and another hydrogenolysis). The title compound was prepared by coupling this partially protected peptide derivative with commercially available 6-(2,5-dioxo-2,5-dihydro-1H-pyrrol-1-ylhexanoic acid in the presence of HATU and N,N-diisopropylethylamine. This was followed by deprotection at the amino group under gentle conditions by stirring for 2.5 hours in 5% strength trifluoroacetic acid in DCM at RT with retention of the ester protective group. Work-up and purification by preparative HPLC gave 438 mg of the title compound.

HPLC (Method 11): Rₜ=1.69 min;

LC-MS (Method 1): Rₜ=0.78 min; MS (ESIpos): m/z=610 (M+H)⁺.

### Intermediate L62

### Trifluoroacetic acid/2-(trimethylsilyl)ethyl N-[6-(2,5-dioxo-2,5-dihydro-1H-pyrrol-1-yl)hexanoyl]-L-valyl-N5-carbamoyl-L-ornithyl-L-lysinate (1:1)

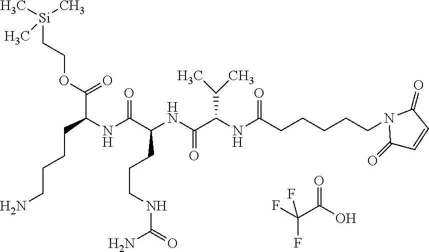 [SMILES: CC(C)[C@H](NC(=O)CCCCCN1C(=O)C=CC1=O)C(=O)N[C@@H](CCCNC(N)=O)C(=O)N[C@@H](CCCCN)C(=O)OCC[Si](C)(C)C ; O=C(O)C(F)(F)F]

First, 2-(trimethylsilyl)ethyl N6-(tert-butoxycarbonyl)-L-lysinate was prepared from N2-[(benzyloxy)carbonyl]-N6-(tert-butoxycarbonyl)-L-lysine according to classical methods of peptide chemistry. 148 mg (0.43 mmol) of this intermediate were then coupled in the presence of 195 mg (0.51 mmol) of HATU and 149 μl of N,N-diisopropylethylamine with 200 mg (0.43 mmol) of Intermediate L16. After concentration and purification of the residue by preparative HPLC, the protected intermediate was taken up in 20 ml of DCM and the tert-butoxycarbonyl protective group was removed by addition of 2 ml of trifluoroacetic acid and 1 h of stirring at RT. Concentration and lyophilization of the residue from acetonitrile/water gave 254 mg (63% of theory over 2 steps).

HPLC (Method 11): Rₜ=1.51 min;

LC-MS (Method 1): Rₜ=0.68 min; MS (ESIpos): m/z=696 (M+H)⁺.

### Intermediate L63

### (4S)-4-{[(2S)-2-{[(2S)-2-{[6-(2,5-Dioxo-2,5-dihydro-1H-pyrrol-1-yl)hexanoyl]amino}-3-methylbutanoyl]amino}propanoyl]amino}-5-oxo-5-[2-(trimethylsilyl)ethoxy]pentanoic acid

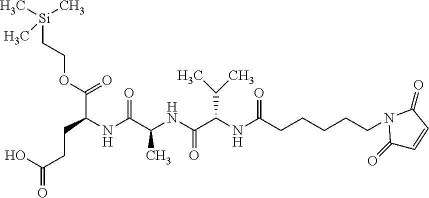 [SMILES: CC(C)[C@H](NC(=O)CCCCCN1C(=O)C=CC1=O)C(=O)N[C@@H](C)C(=O)N[C@@H](CCC(=O)O)C(=O)OCC[Si](C)(C)C]

First, the tripeptide derivative (4S)-4-{[(2S)-2-{[(2S)-2-amino-3-methylbutanoyl]amino}propanoyl]amino}-5-oxo-5-[2-(trimethylsilyl)ethoxy]pentanoic acid was prepared from (2S)-5-(benzyloxy)-2-[(tert-butoxycarbonyl)amino]-5-oxopentanoic acid according to classical methods of peptide chemistry (esterification with 2-(trimethylsilylethanol using EDCI/DMAP, removal of the Boc protective group with trifluoroacetic acid, coupling with N-[(benzyloxy)carbonyl]-L-valyl-L-alanine in the presence of HATU and hydrogenolysis in methanol over 10% palladium on activated carbon). The title compound was prepared by coupling of this partially protected peptide derivative with commercially available 1-{6-[(2,5-dioxopyrrolidin-1-yl)oxy]-6-oxohexyl}-1H-pyrrole-2,5-dione. Work-up and purification by preparative HPLC gave 601 mg of the title compound.

LC-MS (Method 1): Rₜ=0.96 min; MS (ESIpos): m/z=611 (M+H)⁺.

### Intermediate L64

### (4S)-4-{[(2,5-Dioxo-2,5-dihydro-1H-pyrrol-1-yl) acetyl]amino}-5-oxo-5-[2-(trimethylsilyl)ethoxy]pentanoic acid

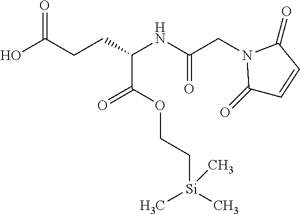 [SMILES: C[Si](C)(C)CCOC(=O)[C@H](CCC(=O)O)NC(=O)CN1C(=O)C=CC1=O]

The title compound was prepared from (2S)-5-(benzyloxy)-2-[(tert-butoxycarbonyl)amino]-5-oxopentanoic acid according to classical methods of peptide chemistry (esterification with 2-(trimethylsilylethanol using EDCI/DMAP, removal of the Boc protective group with trifluoroacetic acid, hydrogenolytic cleavage of the benzyl ester in methanol over 10% palladium on activated carbon and coupling with 1-{2-[(2,5-dioxopyrrolidin-1-yl)oxy]-2-oxoethyl}-1 bi-pyrrole-2,5-dione in the presence of M/V-diisopropylethylamine).

LC-MS (Method 1): Rₜ=0.84 min; MS (ESIpos): m/z=385 (M+H)⁺.

### Intermediate L65

### Trifluoroacetic acid/2-(trimethylsilyl)ethyl 3-{[(benzyloxy)carbonyl]amino}-L-alaninate (1:1)

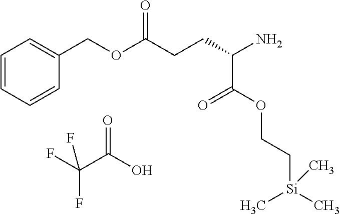 [SMILES: C[Si](C)(C)CCOC(=O)[C@@H](N)CCC(=O)OCc1ccccc1 ; O=C(O)C(F)(F)F]

The title compound was prepared from 3-{[(benzyloxy)carbonyl]amino}-N-(tert-butoxycarbonyl)-L-alanine according to classical methods of peptide chemistry (esterification with 2-(trimethylsilylethanol using EDCI/DMAP and removal of the Boc protective group with trifluoroacetic acid. This gave 373 mg (79% of theory over 2 steps) of the title compound.

LC-MS (Method 1): Rₜ=0.72 min; MS (ESIpos): m/z=339 (M+H)⁺.

### Intermediate L66

### Methyl (8S)-8-(2-hydroxyethyl)-2,2-dimethyl-6,11-dioxo-5-oxa-7,10-diaza-2-silatetradecan-14-oate

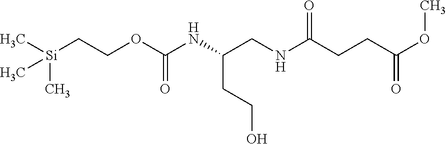

1000 mg (2.84 mmol) of (3S)-3-{[(benzyloxy)carbonyl]amino}-4-[(tert-butoxycarbonyl)amino]butanoic acid were initially charged in 10.0 ml of 1,2-dimethoxyethane, and 344.4 mg (3.4 mmol) of 4-methylmorpholine and 504 mg (3.69 mmol) of isobutyl chloroformate were added. After 10 min of stirring at RT, the reaction was cooled to 5° C. and 161 mg (4.26 mmol) of sodium borohydride dissolved in 3 ml of water were added a little at a time with vigorous stirring. After 1 h, the same amount of sodium borohydride was added again and the reaction was then slowly warmed to RT. 170 ml of water were added and the reaction was then extracted four times with in each case 200 ml of ethyl acetate. The phases were separated and the organic phase was washed once with citric acid and then with saturated sodium bicarbonate solution. The organic phase was dried over magnesium sulphate, the solvent was evaporated under reduced pressure and the residue was dried under high vacuum. This gave 760 mg (78% of theory) of the compound benzyl tert-butyl [(2S)-4-hydroxybutane-1,2-diyl]biscarbamate.

LC-MS (Method 1): Rₜ=0.84 min; MS (ESIpos): m/z=339 (M+H)⁺.

760 mg (2.16 mmol) of this intermediate dissolved in 13 ml of hydrogen chloride/dioxane were stirred at RT for 20 min. The reaction was then concentrated to 5 ml, and diethyl ether was added. The precipitate was filtered off and lyophilized from acetonitrile/water 1:1.

The product obtained in this manner was dissolved in 132 ml of DMF, and 345.5 mg (2.35 mmol) of 4-methoxy-4-oxobutanoic acid, 970 mg (2.55 mmol) of HATU and 1025 μl of N,N-diisopropylethylamine were added. The mixture was stirred at RT for 5 min. The solvent was removed under reduced pressure and the residue that remained was purified by preparative HPLC. The appropriate fractions were combined and the acetonitrile was evaporated under reduced pressure. The aqueous phase that remained was extracted twice with ethyl acetate and the organic phase was then concentrated and dried under high vacuum.

The intermediate obtained in this manner was taken up in methanol and hydrogenated over 10% palladium on activated carbon at RT under hydrogen standard pressure for 1 h. The catalyst was then filtered off and the solvent was removed under reduced pressure.

247 mg of this deprotected compound were taken up in 20 ml of DMF, and 352 mg (1.36 mmol) of 1-({[2-(trimethylsilyl)ethoxy]carbonyl}oxy)pyrrolidine-2,5-dione and 592 μl of N,N-diisopropylethylamine were added. The reaction mixture was stirred at RT for 1 h and then concentrated, and the residue was purified by preparative HPLC. The solvents were then evaporated under reduced pressure and the residue was dried under high vacuum. This gave, over these 5 reaction steps, 218 mg of the title compound in a total yield of 21%.

LC-MS (Method 1): Rₜ=0.74 min; MS (ESIpos): m/z=363 (M+H)⁺.

### Intermediate L67

### Trifluoroacetic acid/2-(2,5-dioxo-2,5-dihydro-1H-pyrrol-1-yl)ethyl-beta-alaninate (1:1)

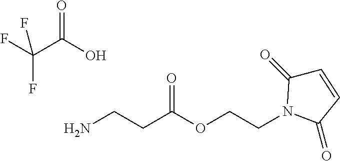 [SMILES: O=C(O)C(F)(F)F ; NCCC(=O)OCCN1C(=O)C=CC1=O]

The title compound was prepared from 50 mg (0.354 mmol) of commercially available 1-(2-hydroxyethyl)-1H-pyrrole-2,5-dione by coupling with 134 mg (0.71 mmol) of N-(tert-butoxycarbonyl)-beta-alanine in 10 ml of dichloromethane in the presence of 1.5 equivalents of EDCI and 0.1 equivalent of 4-M/V-dimethylaminopyridine and subsequent deprotection with trifluoroacetic acid.

Yield: 56 mg (48% of theory over 2 steps)

LC-MS (Method 3): Rₜ=1.15 min; MS (ESIpos): m/z=213 (M+H)⁺.

### Intermediate L68

### Trifluoroacetic acid/N-(2-aminoethyl)-2-(2,5-dioxo-2,5-dihydro-1H-pyrrol-1-ylpropanamide (1:1)

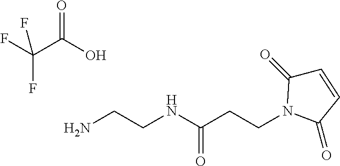 [SMILES: O=C(O)C(F)(F)F ; NCCNC(=O)CCN1C(=O)C=CC1=O]

The title compound was prepared analogously to Intermediate L1 according to classical methods of peptide chemistry from commercially available (2,5-dioxo-2,5-dihydro-1H-pyrrol-1-ylpropanoic acid and tert-butyl (2-aminoethyl)carbamate.

LC-MS (Method 1): Rₜ=0.17 min; MS (ESIpos): m/z=212 (M+H)⁺.

### Intermediate L69

### Trifluoroacetic acid/1-[(benzyloxy)carbonyl]piperidin-4-yl-L-valyl-N5-carbamoyl-L-ornithinate (1:1)

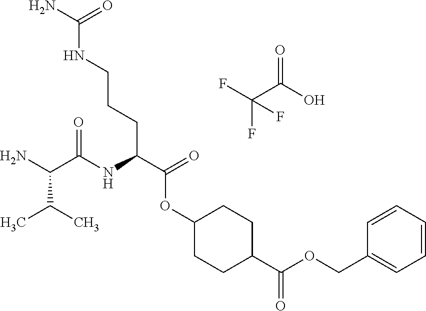 [SMILES: CC(C)[C@H](N)C(=O)N[C@@H](CCCNC(N)=O)C(=O)OC1CCC(C(=O)OCc2ccccc2)CC1 ; O=C(O)C(F)(F)F]

The title compound was prepared by classical methods of peptide chemistry from commercially available benzyl 4-hydroxypiperidine-1-carboxylate by esterification with N2-(tert-butoxycarbonyl)-N5-carbamoyl-L-ornithine using EDCI/DMAP, subsequent Boc removal with TFA, followed by coupling with N-[(tert-butoxy)carbonyl]-L-valine in the presence of HATU and N,N-diisopropylethylamine and finally another Boc removal with TFA.

LC-MS (Method 1): Rₜ=0.62 min; MS (ESIpos): m/z=492 (M+H)⁺.

### Intermediate L70

### 9H-Fluoren-9-ylmethyl (3-oxopropyl)carbamate

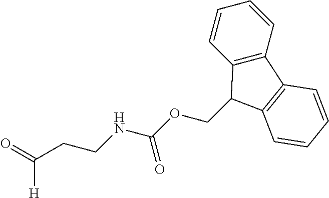 [SMILES: O=CCCNC(=O)OCC1c2ccccc2-c2ccccc21]

1000.0 mg (3.36 mmol) of 9H-fluoren-9-ylmethyl (3-hydroxypropyl)carbamate were initially charged in 15.0 ml of chloroform and 15.0 ml of 0.05 N potassium carbonate/0.05 N sodium bicarbonate solution (1:1). 93.5 mg (0.34 mmol) of tetra-n-butylammonium chloride, 673.6 mg (5.04 mmol) of N-chlorosuccinimide and 52.5 mg (0.34 mmol) of TEMPO were then added and the reaction mixture was stirred vigorously at RT overnight. The reaction mixture was diluted with dichloromethane and the organic phase was washed with water and saturated NaCl solution. The organic phase was dried over magnesium sulphate and the solvent was evaporated under reduced pressure. The residue was dried under high vacuum and purified by silica gel chromatography (mobile phase: cyclohexane/ethyl acetate 3:1-1:1). The solvents were evaporated under reduced pressure and the residue was dried under high vacuum. This gave 589.4 mg (58% of theory) of the title compound.

LC-MS (Method 6): Rₜ=2.15 min; MS (ESIpos): m/z=296 (M−H)⁺.

### Intermediate L71

### tert-Butyl [4-(chlorocarbonyl)phenyl]carbamate

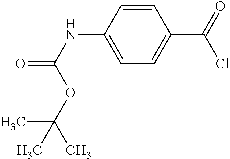 [SMILES: CC(C)(C)OC(=O)Nc1ccc(C(=O)Cl)cc1]

100.0 mg (0.42 mmol) of 4-[(tert-butoxycarbonyl)amino]benzoic acid were initially charged in 2.0 ml of dichloromethane, and 64.2 mg (0.51 mmol) of oxalyl dichloride were added. The reaction mixture was stirred at RT for 30 min (monitored by TLC: dichloromethane/methanol). Another 192.6 mg (1.53 mmol) of oxalyl dichloride and 1 drop of DMF were then added and the mixture was stirred at RT for 1 h. The solvent was evaporated under reduced pressure and the residue was co-distilled repeatedly with dichloromethane. The residue was used without further purification in the next step of the synthesis.

### Intermediate L72

### Benzyl (9S)-9-(hydroxymethyl)-2,2-dimethyl-6,11-dioxo-5-oxa-7,10-diaza-2-silatetradecan-14-oate

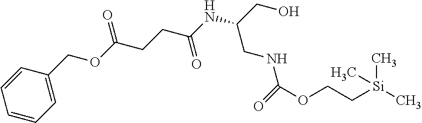 [SMILES: C[Si](C)(C)CCOC(=O)NC[C@@H](CO)NC(=O)CCC(=O)OCc1ccccc1]

The title compound was prepared from commercially available benzyl tert-butyl [(2S)-3-hydroxypropan-1,2-diyl]biscarbamate according to classical methods of peptide chemistry by hydrogenolytic removal of the Z protective group, subsequent coupling with 4-(benzyloxy)-4-oxobutanoic acid in the presence of EDCI/HOBT, followed by removal of the Boc protective group with TFA and finally by reaction with 1-({[2-(trimethylsilyl)ethoxy]carbonyl}oxy)pyrrolidine-2,5-dione in the presence of triethylamine.

LC-MS (Method 1): Rₜ=0.94 min; MS (ESIpos): m/z=425 [M+H]⁺.

### Intermediate L73

### N-(2-Aminoethyl)-6-(2,5-dioxo-2,5-dihydro-1H-pyrrol-1-ylhexanamide

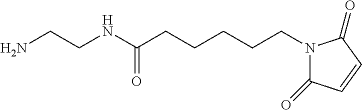 [SMILES: NCCNC(=O)CCCCCN1C(=O)C=CC1=O]

395.5 mg (1.87 mmol) of 6-(2,5-dioxo-2,5-dihydro-1H-pyrrol-1-yl)hexanoic acid, 1.21 g (9.36 mmol) of N,N-diisopropylethylamine and 854.3 mg (2.25 mmol) of HATU were added to a solution of 300 mg (1.87 mmol) of tert-butyl (2-aminoethyl)carbamate in 20 ml of dimethylformamide. The reaction mixture was stirred at RT for 5 minutes. After concentration of the mixture, the residue was taken up in DCM and washed with water. The organic phase was washed with saturated brine, dried over magnesium sulphate, filtered off and concentrated. This gave 408 mg (33%, purity 53%) of the title compound which were used without further purification.

LC-MS (Method 1): Rₜ=0.75 min; MS (ESIpos): m/z=354 (M+H)⁺.

1 ml of TFA was added to a solution of tert-butyl (2-{[6-(2,5-dioxo-2,5-dihydro-1H-pyrrol-1-yl)-hexanoyl]amino}ethyl)carbamate (408 mg, 0.365 mmol) in 7 ml of dichloromethane. The reaction mixture was stirred at RT for 0.5 h. The reaction mixture was concentrated under reduced pressure and the residue was co-distilled twice with dichloromethane. The residue was used further without further purification. This gave 384 mg (94%, purity 57%) of the title compound.

LC-MS (Method 1): Rₜ=0.26 min; MS (ESIpos): m/z=254 (M+H)⁺.

### Intermediate L74

### 3-[2-[2-[2-[2-[[2-(2,5-Dioxopyrrol-1-yl)acetyl]amino]ethoxy]ethoxy]ethoxy]ethoxy]propanoic acid

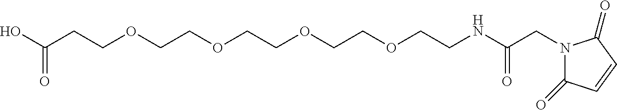 [SMILES: O=C(O)CCOCCOCCOCCOCCNC(=O)CN1C(=O)C=CC1=O]

107 mg (0.335 mmol) of tert-butyl 3-[2-[2-[2-(2-aminoethoxy)ethoxy]ethoxy]ethoxy]propanoate and 93 mg (0.369 mmol) of 2,5-dioxopyrrolidin-1-yl 2-(2,5-dioxopyrrol-1-ylacetate were dissolved in 5 ml of dimethylformamide, and 0.074 ml (0.671 mmol) of N-methylmorpholine were added. The reaction mixture was stirred at RT overnight. 0.048 ml (0.838 mmol) of acetic acid were added and the reaction mixture was purified directly by preparative RP-HPLC (column: Reprosil 125×30; 10μ, flow rate: 50 ml/min, MeCN/water/0.1% TFA). The solvents were evaporated under reduced pressure and the residue was dried under high vacuum. This gave 133 mg (86%, purity 100%) of tert-butyl 3-[2-[2-[2-[2-[[2-(2,5-dioxopyrrol-1-yl) acetyl]amino]ethoxy]ethoxy]ethoxy]ethoxy]propanoate.

LC-MS (Method 1): Rₜ=0.82 min; MS (ESIpos): m/z=459 (M+H)⁺.

0.5 ml of TFA was added to a solution of tert-butyl 3-[2-[2-[2-[2-[[2-(2,5-dioxopyrrol-1-yl-acetyl]amino]ethoxy]ethoxy]ethoxy]ethoxy]propanoate (130 mg, 0.284 mmol) in 5 ml of dichloromethane. The reaction mixture was stirred at RT overnight. The reaction mixture was concentrated under reduced pressure and the residue was taken up in water and lyophilized. The residue was used further without further purification. This gave 102 mg (90%, purity 100%) of the title compound.

LC-MS (Method 1): Rₜ=0.52 min; MS (ESIpos): m/z=402 (M+H)⁺.

### Intermediate L75

### Trifluoroacetic acid/2-(trimethylsilyl)ethyl 3-{[(benzyloxy)carbonyl]amino}-D-alaninate (1:1)

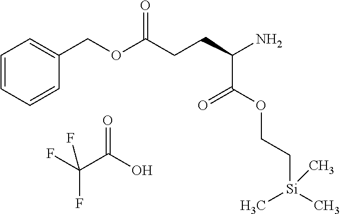 [SMILES: C[Si](C)(C)CCOC(=O)[C@H](N)CCC(=O)OCc1ccccc1 ; O=C(O)C(F)(F)F]

The title compound was prepared from 3-{[(benzyloxy)carbonyl]amino}-N-(tert-butoxycarbonyl)-D-alanine according to classical methods of peptide chemistry (esterification with 2-trimethylsilylethanol using EDCI/DMAP and removal of the Boc protective group with trifluoroacetic acid). This gave 405 mg (58% of theory over 2 steps) of the title compound.

LC-MS (Method 1): Rₜ=0.75 min; MS (ESIpos): m/z=339 (M+H)⁺.

### Intermediate L76

### (2S)-2-Bromo-4-oxo-4-[2-(trimethylsilyl)ethoxy]butanoic acid

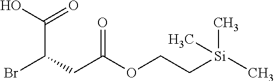

First, a suitably protected aspartic acid derivative was prepared from (3S)-4-(benzyloxy)-3-{[(benzyloxy)carbonyl]amino}-4-oxobutanoic acid according to classical methods of peptide chemistry (esterification with 2-trimethylsilylethanol using EDCI/DMAP and hydrogenolytic removal of the Z protective group and the benzyl ester).

470 mg (1.8 mmol) of the (2S)-2-amino-4-oxo-4-[2-(trimethylsilyl)ethoxy]butanoic acid obtained in this manner were suspended in 10 ml of water, and 1.8 ml of a 1 molar hydrochloric acid and 0.5 ml of concentrated sulphuric acid were added, followed by 863 mg (7.25 mmol) of potassium bromide. At 10° C., a solution of 150 mg (2.175 mmol) of sodium nitrite in 1 ml of water was then added dropwise over a period of 30 min, and the mixture was stirred at 10-15° C. for 2 h. The mixture was then extracted with 50 ml of ethyl acetate. The organic phase was washed with saturated sodium chloride solution and dried over magnesium sulphate. Evaporation of the solvent and purification of the product by preparative HPLC gave 260 mg (48% of theory) of the title compound.

LC-MS (Method 1): Rₜ=1.03 min; MS (ESIneg): m/z=295 and 297 (M−H)⁻.

¹H-NMR (400 MHz, CDCl₃): δ [ppm]=0.03 (s, 9H), 0.95 (t, 2H), 2.94 and 3.2 (2dd, 2H), 4.18 (t, 2H), 4.57 (t, 1H).

### Intermediate L77

### Trifluoroacetic acid/N-[2-(2-Aminoethoxy)ethyl]-2-bromoacetamide (1:1)

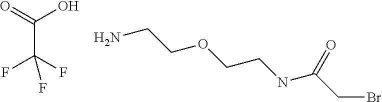 [SMILES: O=C(O)C(F)(F)F ; NCCOCCNC(=O)CBr]

418 mg (2.05 mmol) of tert-butyl [2-(2-aminoethoxy)ethyl]carbamate were initially reacted with 638 mg (2.46 mmol) of bromoacetic anhydride, and the Boc protective group was then removed with trifluoroacetic acid. This gave 551 mg (63% of theory over 2 steps) of the title compound.

LC-MS (Method): Rₜ=0.32 min; MS (ESIpos): m/z=227 and 225 (M+H)⁺.

### Intermediate L78

### N-[(2,5-Dioxo-2,5-dihydro-1H-pyrrol-1-yl)acetyl]-beta-alanine

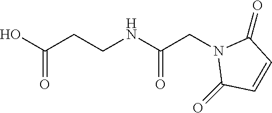 [SMILES: O=C(O)CCNC(=O)CN1C(=O)C=CC1=O]

The title compound was prepared from commercially available (2,5-dioxo-2,5-dihydro-1H-pyrrol-1-yl)acetic acid by coupling with tert-butyl beta-alaninate hydrochloride (1:1) in the presence of EDCI/HOBt and N,N-diisopropylethylamine and subsequent deprotection with trifluoroacetic acid.

LC-MS (Method 1): Rₜ=0.32 min; MS (ESIpos): m/z=227 (M+H)⁺.

### Intermediate L79

### N-[(2,5-Dioxo-2,5-dihydro-1H-pyrrol-1-yl)hexanoyl]-beta-alanine

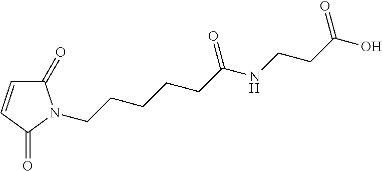 [SMILES: O=C(O)CCNC(=O)CCCCCN1C(=O)C=CC1=O]

64.8 mg (0.357 mmol) of tert-butyl beta-alaninate hydrochloride (1:1) and 100 mg (0.324 mmol) of 1-{6-[(2,5-dioxopyrrolidin-1-yl)oxy]-6-oxohexyl}-1H-pyrrole-2,5-dione were dissolved in 4 ml of dimethylformamide, and 65.6 mg (0.649 mmol) of N-methylmorpholine were added. The reaction mixture was stirred at RT overnight. 0.048 ml (0.838 mmol) of acetic acid were added and the reaction mixture was purified directly by preparative RP-HPLC (column: Reprosil 250×30; 10μ, flow rate: 50 ml/min, MeCN/water/0.1% TFA). The solvents were evaporated under reduced pressure and the residue was dried under high vacuum. This gave 84.5 mg (77%, purity 100%) of tert-butyl N-[6-(2,5-dioxo-2,5-dihydro-1H-pyrrol-1-yl)hexanoyl]-beta-alaninate.

LC-MS (Method 1): Rₜ=0.78 min; MS (ESIpos): m/z=339 (M+H)⁺.

1.62 ml of TFA were added to a solution of tert-butyl N-[6-(2,5-dioxo-2,5-dihydro-1H-pyrrol-1-yl)hexanoyl]-beta-alaninate (82.8 mg, 0.244 mmol) in 8 ml of dichloromethane. The reaction mixture was stirred at RT for 2 hours. The reaction mixture was concentrated under reduced pressure and the residue was taken up in water and lyophilized. The residue was used further without further purification. This gave 62.7 mg (87%, purity 95%) of the title compound.

LC-MS (Method 1): Rₜ=0.75 min; MS (ESIpos): m/z=283 (M+H)⁺.

### Intermediate L80

### 2-(Trimethylsilyl)ethyl 3-[(15-amino-4,7,10,13-tetraoxapentadecan-1-oyl)amino]-N-(tert-butoxycarbonyl)-D-alaninate

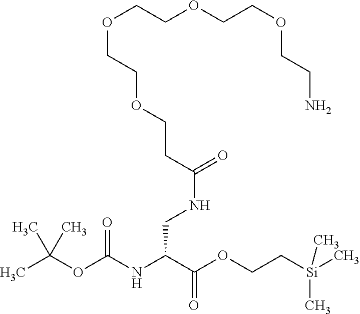 [SMILES: CC(C)(C)OC(=O)N[C@H](CNC(=O)CCOCCOCCOCCOCCN)C(=O)OCC[Si](C)(C)C]

The title compound was prepared from commercially available 3-{[(benzyloxy)carbonyl]amino}-N-(tert-butoxycarbonyl)-D-alanine/N-cyclohexylcyclohexanamine (1:1) according to classical methods of peptide chemistry (release from the salt and esterification with 2-(trimethylsilyl)ethanol using EDCI/DMAP, hydrogenolytic removal of the Z protective group, coupling with commercially available 3-oxo-1-phenyl-2,7,10,13,16-pentaoxa-4-azanonadecan-19-oic acid in the presence of HATU and N,N-diisopropylethylamine and another hydrogenolytic removal of the Z protective group).

LC-MS (Method 1): Rₜ=0.70 min; MS (ESIpos): m/z=552 (M+H)⁺.

### Intermediate L81

### Trifluoroacetic acid/benzyl {2-[(2-aminoethyl) sulphonyl]ethyl}carbamate (1:1)

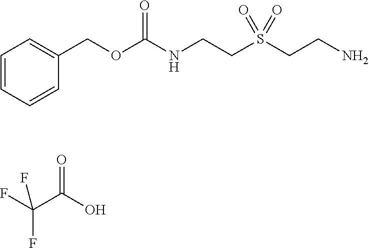 [SMILES: NCCS(=O)(=O)CCNC(=O)OCc1ccccc1 ; O=C(O)C(F)(F)F]

250 mg (1.11 mmol) of 2,2′-sulphonyldiethanamine were coupled with 92.3 mg (0.37 mmol) of 1-{[(benzyloxy)carbonyl]oxy}pyrrolidine-2,5-dione in the presence of N,N-diisopropylethylamine in DMF. Subsequent purification by HPLC gave 70 mg (47% of theory) of the title compound.

LC-MS (Method 12): Rₜ=0.64 min; MS (ESIpos): m/z=257.11 (M+H)⁺.

### Intermediate L82

### Trifluoroacetic acid/N-{2-[2-(2-aminoethoxy)ethoxy]ethyl}-6-(2,5-dioxo-2,5-dihydro-1H-pyrrol-1-yl)hexanamide (1:1)

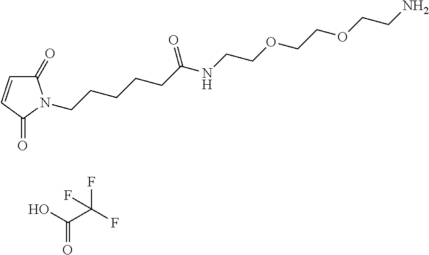 [SMILES: NCCOCCOCCNC(=O)CCCCCN1C(=O)C=CC1=O ; O=C(O)C(F)(F)F]

88.6 mg (0.357 mmol) of N-Boc-2,2′-(ethylenedioxy)diethylamine and 100 mg (0.324 mmol) of N-succinimidyl 6-maleimidohexanoate were dissolved in 4.0 ml of dimethylformamide, and 0.071 ml (0.650 mmol) of N-methylmorpholine were added. The reaction mixture was stirred at RT overnight. 0.048 ml (0.838 mmol) of acetic acid were added and the reaction mixture was purified directly by preparative RP-HPLC (column: Reprosil 125×30; 10μ, flow rate: 75 ml/min, MeCN/water/0.1% TFA). The solvents were evaporated under reduced pressure and the residue was dried under high vacuum. This gave 127 mg (81% of theory) of tert-butyl {2-[2-(2-{[6-(2,5-dioxo-2,5-dihydro-1H-pyrrol-1-yl)hexanoyl]amino}ethoxyethoxy]ethyl}carbamate.

LC-MS (Method 1): Rₜ=0.78 min; MS (ESIpos): m/z=442 (M+H)⁺.

2.0 ml of TFA were added to a solution of 123 mg (225 μmol) tert-butyl {2-[2-(2-{[6-(2,5-dioxo-2,5-dihydro-1H-pyrrol-1-yl)hexanoyl]amino}ethoxyethoxy]ethyl}carbamate in 7.5 ml of dichloromethane. The reaction mixture was stirred at RT for 2 h. The reaction mixture was concentrated under reduced pressure and the residue was taken up in water and lyophilized. The residue was used further without further purification. This gave 111 mg (100% of theory) of the title compound.

LC-MS (Method 1): Rₜ=0.31 min; MS (ESIpos): m/z=342 (M+H)⁺.

¹H-NMR (400 MHz, DMSO-dg): δ [ppm]=1.17 (m, 2H), 1.47 (m, 4H), 2.04 (m, 2H), 2.98 (m, 2H), 3.19 (m, 2H), 3.39 (m, 4H), 3.56 (m, 6H), 7.01 (s, 2H), 7.72 (bs, 3H), 7.80 (m, 1H).

### Intermediate L83

### Trifluoroacetic acid/N-{2-[2-(2-aminoethoxy)ethoxy]ethyl}-2-(2,5-dioxo-2,5-dihydro-1H-pyrrol-1-yl) acetamide (1:1)

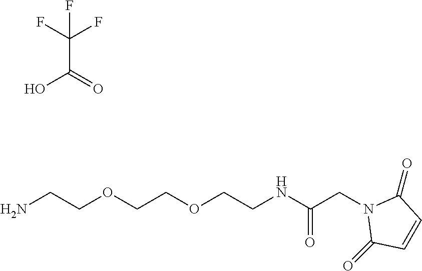 [SMILES: O=C(O)C(F)(F)F ; NCCOCCOCCNC(=O)CN1C(=O)C=CC1=O]

200 mg (0.805 mmol) of tert-butyl {2-[2-(2-aminoethoxy)ethoxy]ethyl}carbamate, 150 mg (0.966 mmol) of (2,5-dioxo-2,5-dihydro-1H-pyrrol-1-ylacetic acid and 560 μl (3.2 mmol) of N,N-diisopropylethylamine were dissolved in 10 ml of dimethylformamide, and 459 mg (1.21 mmol) of HATU were added. The reaction mixture was stirred at RT for 30 minutes. The solvents were evaporated under reduced pressure and the residue was dissolved in dichloromethane. The organic phase was washed twice with 5% strength citric acid solution and dried over magnesium sulphate, and the solvent was evaporated under reduced pressure. The residue was purified using Biotage Isolera (silica gel, column 25 g SNAP, dichloromethane:methanol 98:2). This gave 276 mg (89% of theory) of tert-butyl {2-[2-(2-{[(2,5-dioxo-2,5-dihydro-1H-pyrrol-1-yl)acetyl]amino}ethoxy)-ethoxy]ethyl}carbamate.

LC-MS (Method 1): Rₜ=0.67 min; MS (ESIpos): m/z=386 (M+H)⁺.

4 ml of TFA were added to a solution of tert-butyl {2-[2-(2-{[(2,5-dioxo-2,5-dihydro-1H-pyrrol-1-yl)acetyl]amino}ethoxy)ethoxy]ethyl}carbamate (275 mg, 714 μmol) in 15 ml of dichloromethane. The reaction mixture was stirred at RT for 30 minutes. The reaction mixture was concentrated under reduced pressure and the residue was taken up in water and lyophilized. This gave 281 mg (99% of theory) of the title compound.

LC-MS (Method 1): Rₜ=0.17 min; MS (ESIpos): m/z=286 (M+H)⁺.

### Intermediate L84

### Trifluoroacetic acid/N-(14-amino-3,6,9,12-tetraoxatetradec-1-yl)-6-(2,5-dioxo-2,5-dihydro-1H-pyrrol-1-ylhexanamide (1:1)

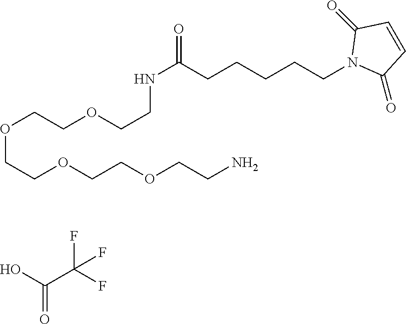 [SMILES: NCCOCCOCCOCCOCCNC(=O)CCCCCN1C(=O)C=CC1=O ; O=C(O)C(F)(F)F]

200 mg (0.594 mmol) of tert-butyl (14-amino-3,6,9,12-tetraoxatetradec-1-yl)carbamate and 202 mg (0.654 mmol) of 1-{6-[(2,5-dioxopyrrolidin-1-yl)oxy]-6-oxohexyl}-1H-pyrrole-2,5-dione were dissolved in 4.0 ml of dimethylformamide, and 0.130 ml (1.2 mmol) of N-methylmorpholine were added. The reaction mixture was stirred at RT overnight. 0.085 ml (1.5 mmol) of acetic acid were added and the reaction mixture was purified directly by preparative RP-HPLC (column: Reprosil 125×30; 10μ, flow rate: 50 ml/min, MeCN/water/0.1% TFA). The solvents were evaporated under reduced pressure and the residue was dried under high vacuum. This gave 275 mg (73% of theory) of tert-butyl [21-(2,5-dioxo-2,5-dihydro-1H-pyrrol-1-yl)-16-oxo-3,6,9,12-tetraoxa-15-azahenicos-1-yl]carbamate.

LC-MS (Method 1): Rₜ=0.81 min; MS (ESIpos): m/z=530 (M+H)⁺.

780 μl (10 mmol) of TFA were added to a solution of tert-butyl [21-(2,5-dioxo-2,5-dihydro-1H-pyrrol-1-yl)-16-oxo-3,6,9,12-tetraoxa-15-azahenicos-1-yl]carbamate (268 mg, 505 μmol) in 5.0 ml of dichloromethane. The reaction mixture was stirred at RT overnight. The reaction mixture was concentrated under reduced pressure and the residue was taken up in water and lyophilized. The residue was used further without further purification. This gave 266 mg (97% of theory) of the title compound.

LC-MS (Method 1): Rₜ=0.46 min; MS (ESIpos): m/z=430 (M+H)⁺.

¹H-NMR (400 MHz, DMSO-dg): δ [ppm]=1.17 (m, 2H), 1.47 (m, 4H), 2.03 (m, 2H), 2.99 (m, 2H), 3.18 (m, 2H), 3.38 (m, 4H), 3.52 (m, 8H), 3.58 (m, 6H), 7.01 (s, 2H), 7.73 (bs, 3H), 7.80 (m, 1H).

### Intermediate L85

### Trifluoroacetic acid/N-(14-amino-3,6,9,12-tetraoxatetradec-1-yl)-2-(2,5-dioxo-2,5-dihydro-1H-pyrrol-1-yl)acetamide (1:1)

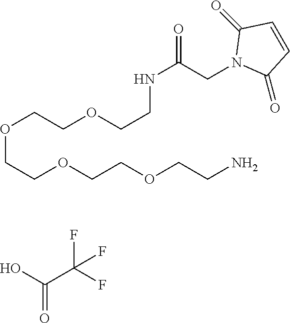 [SMILES: NCCOCCOCCOCCOCCNC(=O)CN1C(=O)C=CC1=O ; O=C(O)C(F)(F)F]

200 mg (0.594 mmol) of tert-butyl (14-amino-3,6,9,12-tetraoxatetradec-1-yl)carbamate, 111 mg (0.713 mmol) of (2,5-dioxo-2,5-dihydro-1H-pyrrol-1-ylacetic acid and 410 μl (2.4 mmol) of N,N-diisopropylethylamine were dissolved in 6 ml of dimethylformamide, and 339 mg (0.892 mmol) of HATU were added. The reaction mixture was stirred at RT for 1 h and purified directly by preparative RP-HPLC (column: Reprosil 250×30; 10μ, flow rate: 50 ml/min, MeCN/water/0.1% TFA). The solvents were evaporated under reduced pressure and the residue was dried under high vacuum. This gave 130 mg (43% of theory) of tert-butyl [17-(2,5-dioxo-2,5-dihydro-1H-pyrrol-1-yl)-16-oxo-3,6,9,12-tetraoxa-15-azaheptadec-1-yl]carbamate.

LC-MS (Method 1): Rₜ=0.71 min; MS (ESIpos): m/z=474 (M+H)⁺.

410 μl (5.3 mmol) of TFA were added to a solution of tert-butyl [17-(2,5-dioxo-2,5-dihydro-1H-pyrrol-1-yl)-16-oxo-3,6,9,12-tetraoxa-15-azaheptadec-1-yl]carbamate (126 mg, 267 μmol) in 4.0 ml of dichloromethane. The reaction mixture was stirred at RT overnight. The reaction mixture was concentrated under reduced pressure and the residue was dried under high vacuum. This gave 124 mg (95% of theory) of the title compound.

LC-MS (Method 13): Rₜ=0.74 min; MS (ESIpos): m/z=374 (M+H)⁺.

¹H-NMR (400 MHz, DMSO-dg): δ [ppm]=2.99 (m, 2H), 3.22 (m, 2H), 3.41 (m, 2H), 3.53 (m, 8H), 3.58 (m, 6H), 4.02 (s, 2H), 7.09 (s, 2H), 7.73 (bs, 3H), 8.21 (m, 1H).

### Intermediate L86

### N-[(2,5-Dioxo-2,5-dihydro-1H-pyrrol-1-yl)acetyl]-L-valyl-L-alanine

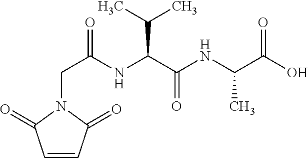 [SMILES: CC(C)[C@H](NC(=O)CN1C(=O)C=CC1=O)C(=O)N[C@@H](C)C(=O)O]

100 mg (0.531 mmol) of L-valyl-L-alanine and 134 mg (0.531 mmol) of l-{2-[(2,5-dioxopyrrolidin-1-yl)oxy]-2-oxoethyl}-1H-pyrrole-2,5-dione were dissolved in 3 ml of dimethylformamide, and 0.150 ml (1.1 mmol) of triethylamine were added. The reaction mixture was stirred at RT for 8 h. The reaction mixture was purified directly by preparative RP-HPLC (column: Reprosil 250×30; 10μ, flow rate: 50 ml/min, MeCN/water). The solvents were evaporated under reduced pressure and the residue was dried under high vacuum. This gave 71.5 mg (41% of theory) of the title compound.

LC-MS (Method 1): Rₜ=0.42 min; MS (ESIpos): m/z=326 (M+H)⁺.

### Intermediate L87

### 3-[2-(2-{[2,5-Dioxo-2,5-dihydro-1H-pyrrol-1-yl)acetyl]amino}ethoxy)ethoxy]propanoic acid

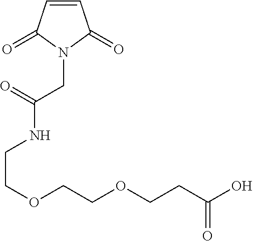 [SMILES: O=C(O)CCOCCOCCNC(=O)CN1C(=O)C=CC1=O]

250 mg (1.07 mmol) of tert-butyl 3-[2-(2-aminoethoxy)ethoxy]propanoate, 151 mg (0.974 mmol) of 2-(2,5-dioxo-2,5-dihydro-1H-pyrrol-1-ylacetic acid, 224 mg (1.46 mmol) of 1-hydroxy-1H-benzotriazole hydrate and 224 mg (1.17 mmol) of 1-(3-dimethylaminopropyl)-3-ethylcarbodiimide hydrochloride were dissolved in 5.0 ml of dimethylformamide. The reaction mixture was stirred at RT for 1 h. Ethyl acetate was added and the mixture was extracted twice with 5% strength citric acid solution and with saturated sodium bicarbonate solution. The organic phase was washed twice with saturated sodium chloride solution and dried over magnesium sulphate, and the solvent was evaporated under reduced pressure. The residue was purified by preparative RP-HPLC (column: Reprosil 250×40; 10μ, flow rate: 50 ml/min, MeCN/water/0.1% TFA). The solvents were evaporated under reduced pressure and the residue was dried under high vacuum. This gave 267 mg (64% of theory) of tert-butyl 3-[2-(2-{[(2,5-dioxo-2,5-dihydro-1H-pyrrol-1-yl-acetyl]amino}ethoxy) ethoxy]propanoate.

LC-MS (Method 1): Rt=0.73 min; MS (ESIpos): m/z=371 (M+H)⁺.

1.1 ml (14 mmol) of TFA were added to a solution of tert-butyl 3-[2-(2-{[(2,5-dioxo-2,5-dihydro-1H-pyrrol-1-yl)acetyl]amino}ethoxy)ethoxy]propanoate (263 mg, 710 μmol) in 10 ml of dichloromethane. The reaction mixture was stirred at RT overnight. The reaction mixture was concentrated under reduced pressure and the residue was dried under high vacuum. This gave 240 mg (94% of theory) of the title compound.

LC-MS (Method 12): Rₜ=0.57 min; MS (ESIpos): m/z=315 (M+H)⁺.

### Intermediate L88

### 2,5-Dioxopyrrolidin-1-yl N-[6-(2,5-dioxo-2,5-dihydro-1H-pyrrol-1-yl)hexanoyl]-L-valyl-L-alaninate

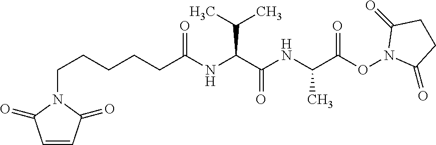 [SMILES: CC(C)[C@H](NC(=O)CCCCCN1C(=O)C=CC1=O)C(=O)N[C@@H](C)C(=O)ON1C(=O)CCC1=O]

150 mg (0.797 mmol) of L-valyl-L-alanine and 246 mg (0.797 mmol) of 1-{6-[(2,5-dioxopyrrolidin-1-yl)oxy]-6-oxohexyl}-1H-pyrrole-2,5-dione were dissolved in 4.0 ml of dimethylformamide, and 0.220 ml (1.6 mmol) of triethylamine were added. The reaction mixture was stirred at RT overnight. The reaction mixture was purified directly by preparative RP-HPLC (column: Reprosil 250×30; 10μ, flow rate: 50 ml/min, MeCN/water). The solvents were evaporated under reduced pressure and the residue was dried under high vacuum. This gave 302 mg (97% of theory) of N-[6-(2,5-dioxo-2,5-dihydro-1H-pyrrol-1-yl)hexanoyl]-L-valyl-L-alanine.

LC-MS (Method 12): Rₜ=1.02 min; MS (ESIpos): m/z=382 (M+H)⁺.

¹H-NMR (400 MHz, DMSO-d₆): δ [ppm]=0.82 (dd, 6H), 1.17 (m, 2H), 1.27 (d, 3H), 1.48 (m, 4H), 1.94 (m, 1H), 2.13 (m, 2H), 3.38 (t, 2H), 4.17 (m, 2H), 7.00 (s, 2H), 7.75 (d, 1H), 8.19 (d, 1H).

130 mg (0.531 mmol) of N-[6-(2,5-dioxo-2,5-dihydro-1H-pyrrol-1-yl)hexanoyl]-L-valyl-L-alanine were dissolved in 6.5 ml of dichloromethane, and 58.8 mg (0.511 mmol) of 1-hydroxypyrrolidine-2,5-dione and 78.4 mg (0.409 mmol) of 1-(3-dimethylaminopropyl)-3-ethylcarbodiimide hydrochloride were added. Another 58.8 mg (0.511 mmol) of 1-hydroxypyrrolidine-2,5-dione and 78.4 mg (0.409 mmol) of 1-(3-dimethylaminopropyl)-3-ethylcarbodiimide hydrochloride were added. Dichloromethane was added and the mixture was washed three times with water. The organic phase was dried over magnesium sulphate, the solvent was evaporated under reduced pressure and the residue was dried under high vacuum. This gave 172 mg (87% of theory) of the title compound.

LC-MS (Method 12): Rₜ=1.28 min; MS (ESIpos): m/z=479 (M+H)⁺.

### Intermediate L89

### 1-Benzyl-5-[2-(trimethylsilyl)ethyl]-L-glutamate hydrochloride (1:1)

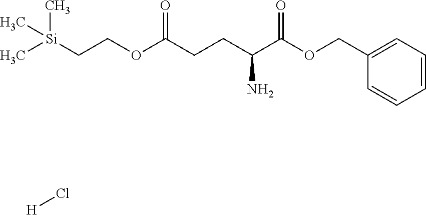 [SMILES: C[Si](C)(C)CCOC(=O)CC[C@H](N)C(=O)OCc1ccccc1 ; Cl]

1.00 g (2.96 mmol) of (4S)-5-(benzyloxy)-4-[(tert-butoxycarbonyl)amino]-5-oxopentanoic acid was initially charged in 13.0 ml of THF, and 510 μl (3.6 mmol) of 2-(trimethylsilyl)ethanol and 109 mg (889 μmol)

of 4-dimethylaminopyridine were added. The reaction mixture was cooled to 0° C., and 682 mg (3.56 mmol) of N-ethyl-N′-3-(dimethylaminopropyl)carbodiimide hydrochloride were added. The reaction mixture was stirred at RT overnight. The solvents were evaporated under reduced pressure and the residue was dissolved in ethyl acetate. The organic phase was washed twice with 0.1 N HCl solution and saturated sodium chloride solution and dried over magnesium sulphate, and the solvent was evaporated under reduced pressure. The residue was purified using Biotage Isolera (silica gel, column 25 g SNAP, cyclohexane:ethyl acetate 80:20). This gave 649 mg (50% of theory) of the compound 1-benzyl-5-[2-(trimethylsilyl)ethyl]-N-(tert-butoxycarbonyl)-L-glutamate.

LC-MS (Method 1): Rₜ=4.6 min; MS (ESIpos): m/z=438 (M+H)⁺.

649 mg (1.48 mmol) of l-benzyl-5-[2-(trimethylsilyl)ethyl]-N-(tert-butoxycarbonyl)-L-glutamate were dissolved in 7.0 ml of dioxane and, with ice bath cooling, 14 ml (59 mmol) of 4N HCl in dioxane were added. The reaction mixture was stirred at RT overnight. The reaction mixture was concentrated under reduced pressure and the residue was dried under high vacuum and purified by Biotage Isolera (silica gel, column 25 g SNAP, dichloromethane:methanol 90:10). This gave 320 mg (57% of theory) of the title compound.

LC-MS (Method 1): Rₜ=0.79 min; MS (ESIpos): m/z=338 (M+H)⁺.

### Intermediate L90

### 1-({N-[(Benzyloxy)carbonyl]glycyl}amino)-3,6,9,12-tetraoxapentadecan-15-oic acid

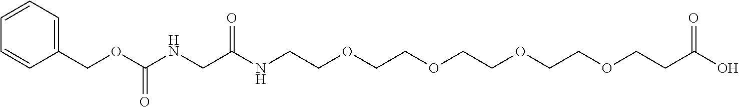 [SMILES: O=C(O)CCOCCOCCOCCOCCNC(=O)CNC(=O)OCc1ccccc1]

118 mg (566 μmol) of N-[(benzyloxy)carbonyl]glycine were initially charged in 5.0 ml of DMF, 200 mg (622 μmol) of tert-butyl 1-amino-3,6,9,12-tetraoxapentadecan-15-oate, 130 mg (849 μmol) of 1-hydroxy-1H-benzotriazole hydrate and 130 mg (679 μmol) of 1-(3-dimethylaminopropyl)-3-ethylcarbodiimide hydrochloride were added and the mixture was stirred at RT for 1 h. Ethyl acetate was added and the mixture was extracted twice with 5% strength citric acid solution and with saturated sodium bicarbonate solution. The organic phase was washed twice with saturated sodium chloride solution and dried over magnesium sulphate. The solvents were evaporated under reduced pressure and the residue was dried under high vacuum. This gave 274 mg (95% of theory) of tert-butyl 1-({N-[(benzyloxy)carbonyl]glycyl}amino)-3,6,9,12-tetraoxapentadecan-15-oate.

LC-MS (Method 12): Rₜ=1.69 min; MS (ESIpos): m/z=513 (M+H)⁺.

820 μl (11 mmol) of TFA were added to a solution of 274 mg (535 μmol) of tert-butyl 1-({N-[(benzyloxy)carbonyl]glycyl}amino)-3,6,9,12-tetraoxapentadecan-15-oate in 5.0 ml of dichloromethane. The reaction mixture was stirred at RT for 3 h. The reaction mixture was concentrated under reduced pressure and the residue was taken up in water and lyophilized. This gave 262 mg (100% of theory) of the title compound.

LC-MS (Method 12): Rₜ=1.12 min; MS (ESIpos): m/z=457 (M+H)⁺.

### Intermediate L91

### Trifluoroacetic acid/2-(trimethylsilyl)ethyl 1-{[3-amino-N-(tert-butoxycarbonyl)-D-alanyl]amino}-3,6,9,12-tetraoxapentadecan-15-oate (1:1)

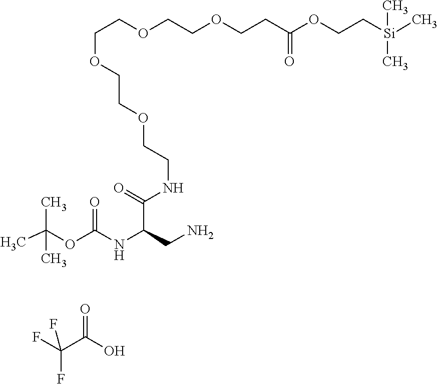 [SMILES: CC(C)(C)OC(=O)N[C@H](CN)C(=O)NCCOCCOCCOCCOCCC(=O)OCC[Si](C)(C)C ; O=C(O)C(F)(F)F]

The title compound was prepared from commercially available 3-oxo-1-phenyl-2,7,10,13,16-pentaoxa-4-azanonadecan-19-oic acid by classical methods of peptide chemistry (esterification with 2-trimethylsilylethanol using EDCI/DMAP, hydrogenolytic removal of the Z protective group, coupling with commercially available N-(tert-butoxycarbonyl)-3-{[(9H-fluoren-9-ylmethoxy)carbonyl]amino}-D-alanine and removal of the Fmoc protective group).

LC-MS (Method 1): Rₜ=0.74 min; MS (ESIpos): m/z=552 (M+H)⁺.

### Intermediate L95

### N-[(Benzyloxy)carbonyl]-L-valyl-L-alanine

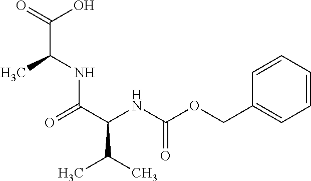 [SMILES: CC(C)[C@H](NC(=O)OCc1ccccc1)C(=O)N[C@@H](C)C(=O)O]

This intermediate was prepared from N-[(benzyloxy)carbonyl]-L-valine and tert-butyl L-alaninate hydrochloride (1:1) using classical methods of peptide chemistry.

LC-MS (Method 12): Rₜ=1.34 min; MS (ESIpos): m/z=323.16 (M+H)⁺.

### Intermediate L96

### N-Acetyl-L-valyl-N⁵-carbamoyl-L-ornithinamide

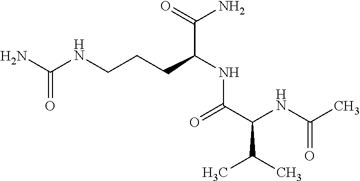 [SMILES: CC(=O)N[C@H](C(=O)N[C@@H](CCCNC(N)=O)C(N)=O)C(C)C]

This intermediate was prepared by classical methods of peptide chemistry starting with the coupling of 2,5-dioxopyrrolidin-1-yl N-[(benzyloxy)carbonyl]-L-valinate with N⁵-carbamoyl-L-ornithine, followed by hydrogenolytic removal of the Z protective group over 10% palladium/activated carbon in ethanol and finally by reaction of the resulting dipeptide with 1-acetoxypyrrolidine-2,5-dione.

LC-MS (Method 1): Rₜ=0.25 min; MS (ESIpos): m/z=317 (M+H)⁺.

### Intermediate L97

### 1-(2,5-Dioxo-2,5-dihydro-1H-pyrrol-1-yl)-2-oxo-6,9,12,15,18,21,24,27-octaoxa-3-azatriacontan-30-oic acid

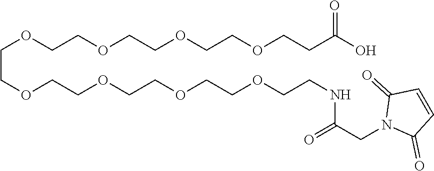 [SMILES: O=C(O)CCOCCOCCOCCOCCOCCOCCOCCOCCNC(=O)CN1C(=O)C=CC1=O]

Tert-Butyl 1-amino-3,6,9,12,15,18,21,24-octaoxaheptacosan-27-oate (100 mg, 201 μmol) was initially charged in 1.0 ml of DMF, and (2,5-dioxo-2,5-dihydro-1H-pyrrol-1-ylacetic acid (46.8 mg, 301 μmol), 1-hydroxy-1H-benzotriazole hydrate (76.9 mg, 502 μmol) and 1-(3-dimethylaminopropyl)-3-ethylcarbodiimide hydrochloride (77.0 mg, 402 μmol) were added. The reaction mixture was stirred at RT overnight, and ethyl acetate was then added. The organic phase was washed twice with 5% strength citric acid solution, with sat. sodium bicarbonate solution and then with sat. sodium chloride solution. The organic phase was dried over magnesium sulphate. The solvents were evaporated under reduced pressure and the residue was purified by prep. RP-HPLC (column: Reprosil 125×30; 10μ, flow rate: 50 ml/min, MeCN/water/0.1% TFA). The solvents were evaporated under reduced pressure and the residue was dried under high vacuum. This gave 19.1 mg (13% of theory) of the compound tert-butyl 1-(2,5-dioxo-2,5-dihydro-1H-pyrrol-1-yl)-2-oxo-6,9,12,15,18,21,24,27-octaoxa-3-azatriacontan-30-oate.

LC-MS (Method 1): Rₜ=0.87 min; MS (ESIpos): m/z=635 [M+H]⁺

TFA (62 μl, 600 μmol) was added to a solution of tert-butyl 1-(2,5-dioxo-2,5-dihydro-1H-pyrrol-1-yl)-2-oxo-6,9,12,15,18,21,24,27-octaoxa-3-azatriacontan-30-oate (19.1 mg, 30.1 μmol) in 1.0 ml of DCM. The reaction mixture was stirred at RT for 3 h. The reaction mixture was concentrated under reduced pressure and the residue was taken up in water and lyophilized. The residue was used further without further purification. This gave 10.8 mg (46% of theory) of the title compound.

LC-MS (Method 1): Rₜ=0.55 min; MS (ESIneg): m/z=577 [M−H]⁻.

### Intermediate L98

### 2,2-Dimethylpropanoic acid/2-(trimethylsilyl)ethyl N-(2-aminoethyl)-N²-{[2-(trimethylsilyl)ethoxy]carbonyl}-L-glutaminate (1:1)

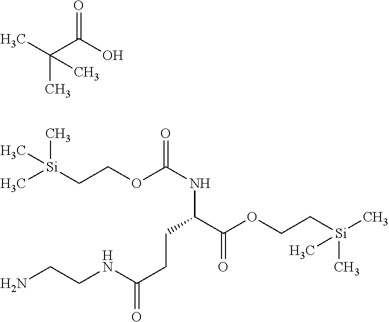

First, (4S)-5-tert-butoxy-4-[(tert-butoxycarbonyl)amino]-5-oxopentanoic acid was coupled in the presence of HATU and N,N-diisopropylethylamine with benzyl (2-aminoethyl)carbamate. Then, the Boc protective group and the tert-butyl ester were cleaved using trifluoroacetic acid in DCM. Subsequently, first the amino group was protected again by reaction with 1-({[2-(trimethylsilyl)ethoxy]carbonyl}oxy)pyrrolidine-2,5-dione in DMF/water in the presence of N,N-diisopropylethylamine, and then the carboxyl group by reaction with 2-(trimethylsilyl)ethanol in DCM in the presence of EDCI/DMAP. In the last step, the terminal amino group was deprotected by hydrogenolysis over 10% palladium on activated carbon in ethanol under standard pressure. Removal of the catalyst by filtration, concentration, purification by preparative HPLC and freeze-drying of the residue from acetonitrile/water gave the title compound.

LC-MS (Method 1): Rₜ=0.82 min; MS (ESIpos): m/z=434 (M+H)⁺.

### Intermediate L99

### Trifluoroacetic acid/2-(trimethylsilyl)ethyl N-[(2,5-dioxo-2,5-dihydro-1H-pyrrol-1-yl)acetyl]-L-valyl-L-alanyl-beta-alanyl-L-lysinate (1:1)

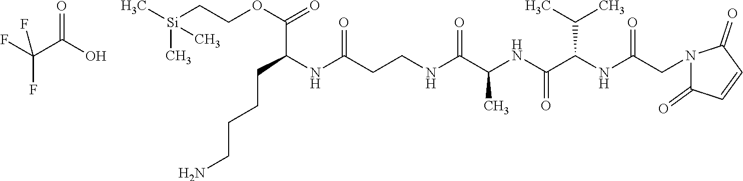 [SMILES: O=C(O)C(F)(F)F ; CC(C)[C@H](NC(=O)CN1C(=O)C=CC1=O)C(=O)N[C@@H](C)C(=O)NCCC(=O)N[C@@H](CCCCN)C(=O)OCC[Si](C)(C)C]

First, starting from N2-[(benzyloxy)carbonyl]-N6-(tert-butoxycarbonyl)-L-lysine, 2-(trimethylsilyl)ethyl N6-(tert-butoxycarbonyl)-L-lysinate was prepared by classical methods of peptide chemistry. This intermediate was then coupled in the presence of HATU and N,N-diisopropylethylamine using the tripeptide building block N-[(benzyloxy)carbonyl]-L-valyl-L-alanyl-beta-alanine, which had been prepared by standard methods. The Z protective group was subsequently removed by hydrogenolysis and methanol and the resulting intermediate was coupled with (2,5-dioxo-2,5-dihydro-1H-pyrrol-1-ylacetic acid in the presence of HATU and N,N-diisopropylethylamine. In the last step, the side chain amino group was deprotected under mild conditions by stirring in 10% strength trifluoroacetic acid in DCM at RT for 1 h. Concentration and freeze-drying from acetonitrile/water gave the title compound.

LC-MS (Method 1): Rₜ=0.64 min; MS (ESIpos): m/z=625 (M+H)⁺.

### Intermediate L100

### 3-[5-(2-{[(2,5-Dioxo-2,5-dihydro-1H-pyrrol-1-yl)acetyl]amino}ethyl)-1,2,4-oxadiazol-3-yl]propanoic acid

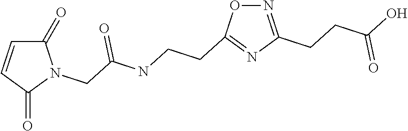 [SMILES: O=C(O)CCc1noc(CCNC(=O)CN2C(=O)C=CC2=O)n1]

461 mg (6.60 mmol) of hydroxylamine hydrochloride and 1341.86 mg (13.26 mmol) of triethylamine were added to a solution of methyl 3-cyanopropanoate (500 mg, 4.42 mmol) in 40 ml of ethanol. The reaction mixture was stirred at 50° C. for 3 h. The mixture was concentrated and the residue was dissolved in ethyl acetate and then washed with water and brine. The organic phase was dried over magnesium sulphate and concentrated. The residue was used without further purification. This gave 400 mg (62% of theory) of the title compound.

6.91 g (36.50 mmol) of N-(tert-butoxycarbonyl)-beta-alanine and 8.22 g (39.82 mmol) of 1,3-dicyclohexylcarbodiimide were added to a solution of methyl (4E)-4-{[N-(tert-butoxycarbonyl)-beta-alanyl]amino}-4-(hydroxyimino)butanoate (4.85 g, 33.19 mmol) in 120.0 ml of dioxane. The reaction mixture was stirred at room temperature for 3 h. The mixture was concentrated and the residue was dissolved in water and extracted with ethyl acetate. The organic phase was dried over sodium sulphate and concentrated. The residue was purified by flash chromatography. This gave 6.0 g (57% of theory) of the title compound.

A solution of methyl (4E)-4-{[N-(tert-butoxycarbonyl)-beta-alanyl]amino}-4-(hydroxyimino)butanoate (6.0 g, 18.91 mmol) in 100 ml of DMF was stirred at 120° C. for 5 h. Water was added and the mixture was extracted with ethyl acetate. The organic phase was dried over sodium sulphate and concentrated. The residue was purified by prep. HPLC. This gave 4 g (71% of theory) of the title compound.

2.96 g (25.96 mmol) of trifluoroacetic acid were added to a solution of 3-(5-{2-[(tert-butoxycarbonyl)amino]ethyl}-1,2,4-oxadiazol-3-yl)propanoic acid (2.0 g, 7.01 mmol) in 30 ml of dichloromethane. The reaction mixture was stirred at room temperature for 1 h. Water was added and the mixture was extracted with dichloromethane. The organic phase was dried over sodium sulphate and concentrated. The residue was used without further purification. This gave 1.50 g (72% of theory) of the title compound.

1.30 g (5.52 mmol) of 1-[2-(2,5-dioxopyrrolidin-1-yl)-2-oxoethyl]-1-pyrrole-2,5-dione and 1.52 g (15.04 mmol) of triethylamine were added to a solution of 3-[5-(2-aminoethyl)-1,2,4-oxadiazol-3-yl]propanoic acid (1.5 g, 5.01 mmol) in 25 ml of DMF. The reaction mixture was stirred at RT for 1 h. Water was added and the mixture was extracted with dichloromethane. The organic phase was dried over sodium sulphate and concentrated. The residue was purified by prep. HPLC. This gave 774 mg (47% of theory) of the title compound.

¹H-NMR (300 MHz, DMSO-d₆): δ [ppm]=2.67 (t, 2H), 2.91 (t, 2H), 3.03 (t, 2H), 3.46 (q, 2H), 4.28 (s, 2H), 7.01 (s, 2H), 8.37 (t, 1H), 12.28 (bs, 1H).

### Intermediate L123

### tert-Butyl [1-fluoro-4-oxobutan-2-yl]carbamate

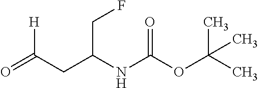

Under argon, ethyl 3-[(tert-butoxycarbonyl)amino]-4-fluorobutanoate (150 mg, 602 μmol) (Synth. Com., 1985, 15(5), 377) was initially charged in 12.0 ml of DCM. The reaction mixture was cooled to −78° C., diisobutylaluminium hydride 1M in toluene (1.2 ml, 1.0 M, 1.2 mmol) was added and the mixture was stirred for 2 hours. The mixture was carefully quenched with methanol, stirred for another 10 min and diluted with ethyl acetate. The organic phase was extracted three times with sat. potassium sodium tartrate solution. The organic phase was washed once with sat. NaCl solution and dried over magnesium sulphate. The solvent was evaporated under reduced pressure and the residue was dried under high vacuum. This gave 86.1 mg (67% of theory) of the title compound. ¹H-NMR (400 MHz, DMSO-d6) δ [ppm]: 1.37 (s, 9H), 2.58 (m, 2H), 4.18 (m, 1H), 4.31 (dd, 2H), 7.05 (d, 1H), 9.60 (s, 1H)

### Intermediate L124

### tert-Butyl N-{(2R)-2-amino-3-oxo-3-[2-(trimethylsilyl)ethoxy]propyl}-N²-(tert-butoxycarbonyl)-L-aspartate

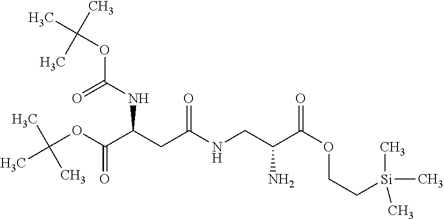 [SMILES: CC(C)(C)OC(=O)N[C@@H](CC(=O)NC[C@@H](N)C(=O)OCC[Si](C)(C)C)C(=O)OC(C)(C)C]

4.0 g (13.8 mmol) of Boc-Asp-OtBu and 1.8 g (15.2 mmol) of N-hydroxysuccinimide were dissolved in 100 ml of ethyl acetate, and 3.1 g (15.2 mmol) of 1,3-dicyclohexylcarbodiimide were added at 0° C. The reaction mixture was stirred at 0° C. for 2 hours and then at RT overnight. The reaction mixture was subsequently filtered and concentrated under reduced pressure. This gave 4.1 g (77% of theory) of the compound 1-tert-butyl 4-(2,5-dioxopyrrolidin-1-yl)-N-(tert-butoxycarbonyl)-L-aspartate.

3-Amino-N-[(benzyloxy)carbonyl]-D-alanine (2.53 g, 10.6 mmol) was dissolved in 30 ml of DMF, and N,N-diisopropylethylamine (2.74 g, 21.2 mmol) and 1-tert-butyl 4-(2,5-dioxopyrrolidin-1-yl)-N-(tert-butoxycarbonyl)-L-aspartate (4.10 g, 10.6 mmol) were added. The reaction mixture was stirred at RT overnight and concentrated under reduced pressure. This gave 4.9 g (90% of theory) of the compound (2R)-2-{[(benzyloxy)carbonyl]amino}-3-({(3S)-4-tert-butoxy-3-[(tert-butoxycarbonyl)amino]-4-oxobutanoyl}aminopropanoic acid.

(2R)-2-{[(Benzyloxy)carbonyl]amino}-3-({(3S)-4-tert-butoxy-3-[(tert-butoxycarbonyl)amino]-4-oxobutanoyl}aminopropanoic acid (4.90 g, 9.62 mmol) was dissolved in 100 ml of acetonitrile, and pyridine (1.6 ml, 19 mmol), 2-(trimethylsilyl)ethanol (1.7 ml, 12 mmol) and dicyclohexylcarbodiimide (2.38 g, 11.5 mmol) were added at RT. The reaction mixture was stirred at 0° C. for 1 hour and then at RT overnight. The reaction mixture was subsequently filtered and concentrated under reduced pressure. The residue was purified by prep. RP-HPLC. The solvents were evaporated under reduced pressure and the residue was dried under high vacuum. This gave 3.9 g (66% of theory) of the compound tert-butyl N-{(2R)-2-{[(benzyloxy)carbonyl]amino}-3-oxo-3-[2-(trimethylsilyl)ethoxy]propyl}-N2-(tert-butoxycarbonyl)-L-aspartate.

tert-Butyl N-{(2R)-2-{[(benzyloxy)carbonyl]amino}-3-oxo-3-[2-(trimethylsilyl)ethoxy]propyl}-N²-(tert-butoxycarbonyl)-L-aspartate (3.80 g, 6.23 mmol) was dissolved in 120 ml of methanol, and 380 mg of palladium on carbon (10%) were added. The reaction mixture was hydrogenated at RT with hydrogen under standard pressure for 2 hours and then filtered. The solvent was removed under reduced pressure. This gave 2.9 g (84% of theory) of the title compound

¹H-NMR (400 MHz, DMSO-d₆): δ [ppm]=0.04 (s, 9H), 0.97 (m, 2H), 1.38 (s, 9H), 1.39 (s, 9H), 1.89 (bs, 2H), 2.43 (m, 1H), 3.18 (m, 3H), 3.38 (m, 1H), 4.11 (m, 3H), 6.93 (d, 1H), 7.91 (bt, 1H)

### Intermediate L125

### Trifluoroacetic acid/tert-butyl-N-(2-aminoethyl)-N²-(bromoacetyl)-D-alpha-glutaminate (1:1)

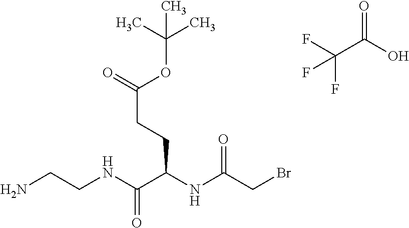 [SMILES: CC(C)(C)OC(=O)CC[C@@H](NC(=O)CBr)C(=O)NCCN ; O=C(O)C(F)(F)F]

This intermediate was prepared starting with (2R)-2-{[(benzyloxy)carbonyl]amino}-5-tert-butoxy-5-oxopentanoic acid and tert-butyl (2-aminoethyl)carbamate by classical methods of peptide chemistry.

LC-MS (Method 1): Rₜ=0.49 min; MS (ESIpos): m/z=366 und 368 (M+H)⁺.

### Intermediate F104

### Trifluoroacetic acid/(2S)-2-amino-4-[{(1R)-1-[1-benzyl-4-(2,5-difluorophenyl)-1H-pyrrol-2-yl]-2,2-dimethylpropyl}(glycoloyl)amino]-N-(2-{[(2,5-dioxo-2,5-dihydro-1H-pyrrol-1-yl-acetyl]amino}ethylbutanamide (1:1)

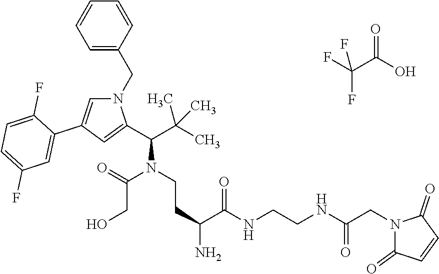 [SMILES: CC(C)(C)[C@H](c1cc(-c2cc(F)ccc2F)cn1Cc1ccccc1)N(CC[C@H](N)C(=O)NCCNC(=O)CN1C(=O)C=CC1=O)C(=O)CO ; O=C(O)C(F)(F)F]

10 mg (0.014 mmol) of Intermediate C53 were dissolved in 3.3 ml of DMF, and 8.5 mg (0.027 mmol) of Intermediate L1, 7.8 mg (0.02 mmol) of HATU and 12 μl of N,N-diisopropylethylamine were added. The reaction was stirred at RT for 15 min and then concentrated. The residue was purified by preparative HPLC giving, after lyophilization, 5.6 mg (38% of theory) of the protected intermediate.

LC-MS (Method 1): Rₜ=1.32 min; MS (ESIpos): m/z=915 (M+H)⁺.

5.6 mg (0.006 mmol) of this intermediate were taken up in 2 ml of DMF, and 69 mg (0.61 mmol) of 1,4-diazabicyclo[2.2.2]octane were added. The reaction was treated in an ultrasonic bath for 2 h. 35 μl of acetic acid were then added and the reaction was concentrated under high vacuum. The residue was purified by preparative HPLC. This gave 2.4 mg (48% of theory) of the title compound.

LC-MS (Method 1): Rₜ=0.84 min; MS (EIpos): m/z=693 [M+H]⁺.

HPLC (Method 11): Rₜ=1.91 min;

Alternatively, the title compound was also prepared from Intermediate C58. 15 mg (0.023 mmol) of Intermediate C58 were initially reacted with 11 mg (0.036 mmol) of Intermediate L1 in the presence of 13 mg (0.034 mmol) of HATU and 10 μl of N,N-diisopropylethylamine. After 60 min of stirring at RT, the mixture was concentrated and the residue was purified by preparative HPLC. This gave 12.3 mg (63% of theory) of the protected intermediate.

LC-MS (Method 1): Rₜ=1.3 min; MS (EIpos): m/z=837 [M+H]⁺.

In the second step, this intermediate was dissolved in 3 ml of 2,2,2-trifluoroethanol. 12 mg (0.088 mmol) of zinc chloride were added, and the reaction was stirred at 50° C. for 2 h. 26 mg (0.088 mmol) of ethylenediamine-N,N,N′,N′-tetraacetic acid and 2 ml of a 0.1% strength aqueous trifluoroacetic acid solution were then added. The reaction was purified by preparative HPLC. Concentration of the appropriate fractions and lyophilization of the residue from acetonitrile/water gave 8.1 mg (68% of theory) of the title compound.

LC-MS (Method 1): Rₜ=0.89 min; MS (ESIpos): m/z=693 (M+H)⁺.

### Intermediate F119

### Trifluoroacetic acid/(2S)-2-amino-4-[{(1R)-1-[1-benzyl-4-(2,5-difluorophenyl)-1H-pyrrol-2-yl]-2,2-dimethylpropyl}(glycoloyl)amino]-N-{2-[(bromoacetyl)amino]ethyl}butanamide (1:1)

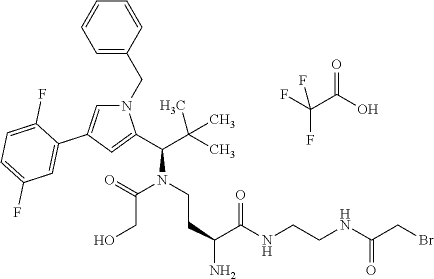 [SMILES: CC(C)(C)[C@H](c1cc(-c2cc(F)ccc2F)cn1Cc1ccccc1)N(CC[C@H](N)C(=O)NCCNC(=O)CBr)C(=O)CO ; O=C(O)C(F)(F)F]

29 mg (0.044 mmol) of Intermediate C58 were taken up in 3.4 ml of DMF, and 36 mg (0.087 mmol) of Intermediate L52, 25 mg (0.065 mmol) of HATU and 19 μl of N,N-diisopropylethylamine were added. After 60 min of stirring at RT, the mixture was concentrated and the residue was purified by preparative HPLC. This gave 26.4 mg (73% of theory) of the intermediate.

LC-MS (Method 1): Rₜ=1.34 min; MS (ESIpos): m/z=820 and 822 (M+H)⁺.

This intermediate was dissolved in 3 ml of 2,2,2-trifluoroethanol. 6.5 mg (0.048 mmol) of zinc chloride were added, and the reaction was stirred at 50° C. for 4 h. 13.9 mg (0.048 mmol) of ethylenediamine-N,N,N′,N′-tetraacetic acid and 2 ml of a 0.1% strength aqueous trifluoroacetic acid solution were added. The reaction was purified by preparative HPLC. Concentration of the appropriate fractions and lyophilization of the residue from acetonitrile/water gave 14.4 mg (58% of theory) of the title compound.

LC-MS (Method 1): Rₜ=0.88 min; MS (ESIpos): m/z=676 and 678 (M+H)⁺.

### Intermediate F127

### Trifluoroacetic acid/(2S)-2-amino-4-({(1R)-1-[1-benzyl-4-(2,5-difluorophenyl)-1H-pyrrol-2-yl]-2,2-dimethylpropyl}[(2S)-2-methoxypropanoyl]amino)-N-(2-{[(2,5-dioxo-2,5-dihydro-1H-pyrrol-1-yl) acetyl]amino}ethyl)butanamide (1:1)

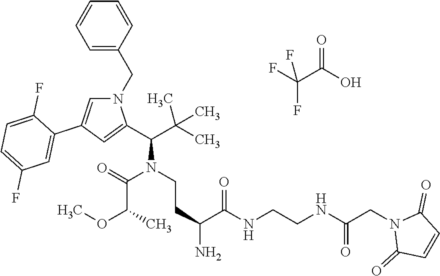 [SMILES: CO[C@@H](C)C(=O)N(CC[C@H](N)C(=O)NCCNC(=O)CN1C(=O)C=CC1=O)[C@@H](c1cc(-c2cc(F)ccc2F)cn1Cc1ccccc1)C(C)(C)C ; O=C(O)C(F)(F)F]

12 mg (0.015 mmol) of Intermediate C59 were dissolved in 2.4 ml of DMF, and 14.6 mg (0.046 mmol) of Intermediate L1, 6 mg (0.031 mmol) of 1-(3-dimethylaminopropyl)-3-ethylcarbodiimide hydrochloride, 5.9 mg (0.039 mmol) of 1-hydroxy-1H-benzotriazole hydrate and 8 μl of N,N-diisopropylethylamine were added. After 1 h of stirring at RT, the mixture was concentrated and the residue was purified by preparative HPLC. This gave 11 mg (70% of theory) of this intermediate.

LC-MS (Method 1): Rₜ=1.34 min; MS (ESIpos): m/z=942 (M+H)⁺.

11 mg (0.011 mmol) of this intermediate were taken up in 2 ml of DMF, and 123 mg (1.1 mmol) of 1,4-diazabicyclo[2.2.2]octane were added. The reaction was treated in an ultrasonic bath for 2 h. 63 μl of acetic acid were then added and the reaction was concentrated under high vacuum. The residue was purified by preparative HPLC. This gave 2 mg (22% of theory) of the title compound.

LC-MS (Method 1): Rₜ=0.89 min; MS (EIpos): m/z=721 [M+H]⁺.

HPLC (Method 11): Rₜ=1.95 min;

### Intermediate F153

### Trifluoroacetic acid/(2S)-2-amino-4-({(1R)-1-[1-benzyl-4-(2,5-difluorophenyl)-1H-pyrrol-2-yl]-2,2-dimethylpropyl}[(2S)-2-hydroxypropanoyl]amino)-N-(2-{[(2,5-dioxo-2,5-dihydro-1H-pyrrol-1-yl) acetyl]amino}ethylbutanamide (1:1)

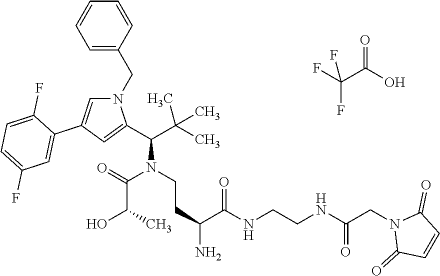 [SMILES: C[C@H](O)C(=O)N(CC[C@H](N)C(=O)NCCNC(=O)CN1C(=O)C=CC1=O)[C@@H](c1cc(-c2cc(F)ccc2F)cn1Cc1ccccc1)C(C)(C)C ; O=C(O)C(F)(F)F]

The synthesis was carried out analogously to Intermediate F104 from Intermediate C60.

LC-MS (Method 1): Rₜ=1.1 min; MS (ESIpos): m/z=707 (M+H)⁺.

### Intermediate F155

### N⁶—(N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-beta-alanyl)-N²—{N-[6-(2,5-dioxo-2,5-dihydro-1H-pyrrol-1-yl)hexanoyl]-L-valyl-L-alanyl}-L-lysine/trifluoroacetic acid (1:1)

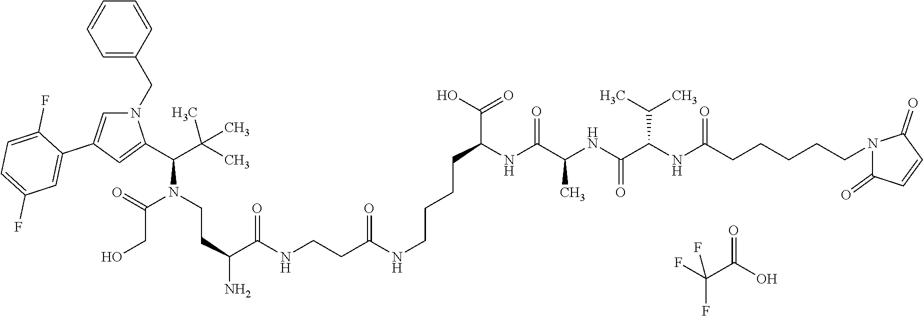 [SMILES: CC(C)[C@H](NC(=O)CCCCCN1C(=O)C=CC1=O)C(=O)N[C@@H](C)C(=O)N[C@@H](CCCCNC(=O)CCNC(=O)[C@@H](N)CCN(C(=O)CO)[C@@H](c1cc(-c2cc(F)ccc2F)cn1Cc1ccccc1)C(C)(C)C)C(=O)O ; O=C(O)C(F)(F)F]

The title compound was prepared by coupling of 14 mg (0.019 mmol) of Intermediate C61 with 15 mg (0.021 mmol) of Intermediate L61 in the presence of 8.7 mg (0.023 mmol) of HATU and 17 μl of N,N-diisopropylethylamine and subsequent deprotection with zinc chloride in trifluoroethanol as described for Intermediate F119. Purification by preparative HPLC gave 13 mg (59% of theory over 2 steps) of the title compound.

LC-MS (Method 1): Rₜ=0.86 min; MS (ESIpos): m/z=1076 (M+H)⁺.

### Intermediate F173

### N-[6-(2,5-Dioxo-2,5-dihydro-1H-pyrrol-1-yl)hexanoyl]-L-valyl-L-alanyl-N-[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]-L-glutamine/trifluoroacetic acid (1:1)

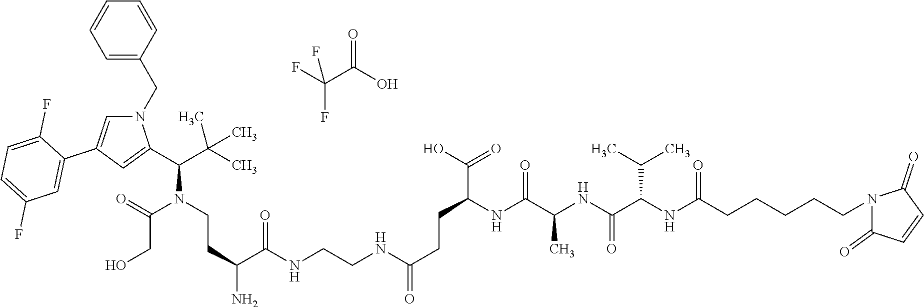 [SMILES: CC(C)[C@H](NC(=O)CCCCCN1C(=O)C=CC1=O)C(=O)N[C@@H](C)C(=O)N[C@@H](CCC(=O)NCCNC(=O)[C@@H](N)CCN(C(=O)CO)[C@@H](c1cc(-c2cc(F)ccc2F)cn1Cc1ccccc1)C(C)(C)C)C(=O)O ; O=C(O)C(F)(F)F]

The title compound was prepared from 15 mg (0.018 mmol) of Intermediate C64 by coupling with 12 mg (0.02 mmol) of Intermediate L63 in the presence of 7.7 mg (0.02 mmol) of HATU and 16 μl of N,N-diisopropylethylamine and subsequent deprotection with zinc chloride in trifluoroethanol as described for Intermediate F119. Purification by preparative HPLC gave 12 mg (58% of theory over 2 steps) of the title compound.

LC-MS (Method 1): Rₜ=0.91 min; MS (EIpos): m/z=1048 [M+H]⁺.

### Intermediate F178

### Trifluoroacetic acid/(1R,2S)-2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)-N-{2-[(bromoacetyl)amino]-ethyl}cyclopentanecarboxamide (1:1)

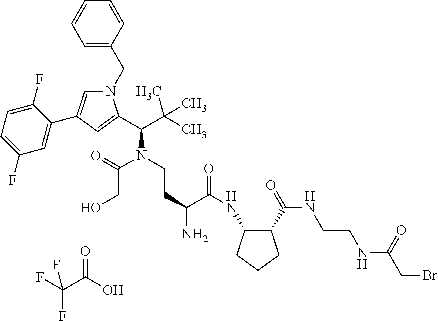 [SMILES: CC(C)(C)[C@H](c1cc(-c2cc(F)ccc2F)cn1Cc1ccccc1)N(CC[C@H](N)C(=O)N[C@H]1CCC[C@H]1C(=O)NCCNC(=O)CBr)C(=O)CO ; O=C(O)C(F)(F)F]

The title compound was prepared analogously to Intermediate FI 77 using, instead of Intermediate L1, the Intermediate L52.

LC-MS (Method 1): Rₜ=0.89 min; MS (EIpos): m/z=787 and 789 [M+H]⁺.

### Intermediate F180

### N-[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]-N2-[(2,5-dioxo-2,5-dihydro-1H-pyrrol-1-yl)acetyl]-L-glutamine/trifluoroacetic acid (1:1)

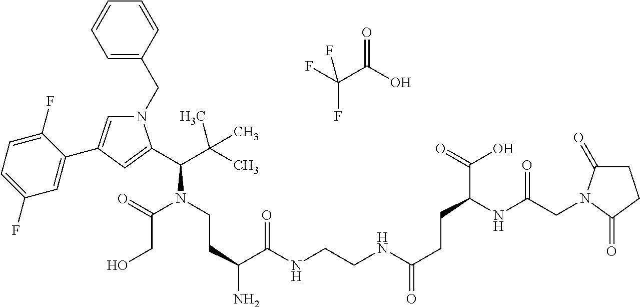 [SMILES: CC(C)(C)[C@H](c1cc(-c2cc(F)ccc2F)cn1Cc1ccccc1)N(CC[C@H](N)C(=O)NCCNC(=O)CC[C@H](NC(=O)CN1C(=O)CCC1=O)C(=O)O)C(=O)CO ; O=C(O)C(F)(F)F]

The title compound was prepared by coupling of 9.6 mg (0.012 mmol) of Intermediate C64 with 5 mg (0.013 mmol) of Intermediate L64 in the presence of 7 mg (0.018 mmol) of HATU and 6 μl of N,N-diisopropylethylamine and subsequent deprotection with zinc chloride in trifluoroethanol as described for Intermediate F119. Purification by preparative HPLC gave 3.1 mg (28% of theory over 2 steps) of the title compound.

LC-MS (Method 1): Rₜ=0.85 min; MS (EIpos): m/z=822 [M+H]⁺.

### Intermediate F192

### N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-3-{[(2,5-dioxo-2,5-dihydro-1H-pyrrol-1-yl)acetyl]amino}-L-alanine/trifluoroacetic acid (1:1)

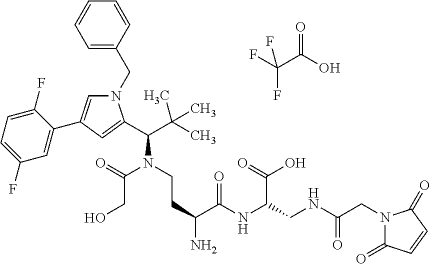

60 mg (0.091 mmol) of Intermediate C58 were taken up in 8 ml of DMF and coupled with 45 mg (0.100 mmol) of Intermediate L65 in the presence of 42 mg (0.11 mmol) of HATU and 64 μl of N,N-diisopropylethylamine. After purification by preparative HPLC, the intermediate was taken up in 10 ml of ethanol and hydrogenated over 10% palladium on activated carbon at RT under hydrogen standard pressure for 45 min. The catalyst was then filtered off, the solvent was removed under reduced pressure and the product was purified by preparative HPLC. Lyophilization from acetonitrile/water 1:1 gave 24.5 mg (31% of theory over 2 steps) of 2-(trimethylsilyl)ethyl 3-amino-N-[(2S)-4-[{(1R)-1-[1-benzyl-4-(2,5-difluorophenyl)-1H-pyrrol-2-yl]-2,2-dimethylpropyl}(glycoloyl)amino]-2-({[2-(trimethylsilyl)ethoxy]carbonyl}aminobutanoyl]-L-alaninate.

LC-MS (Method 1): Rₜ=1.17 min; MS (EIpos): m/z=844 [M+H]⁺.

The title compound was then prepared by coupling of 10 mg (0.012 mmol) of this intermediate with 2 mg (0.013 mmol) of commercially available (2,5-dioxo-2,5-dihydro-1H-pyrrol-1-ylacetic acid intermediate in the presence of 5.4 mg (0.014 mmol) of HATU and 8 μl of N,N-diisopropylethylamine and subsequent deprotection with zinc chloride in trifluoroethanol as described for Intermediate F119. Purification by preparative HPLC gave 3.5 mg (33% of theory over 2 steps) of the title compound.

LC-MS (Method 1): Rₜ=0.81 min; MS (ESIpos): m/z=737 (M+H)⁺.

### Intermediate F193

### N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-3-{[(2,5-dioxo-2,5-dihydro-1H-pyrrol-1-yl)acetyl]amino}-D-alanine/trifluoroacetic acid (1:1)

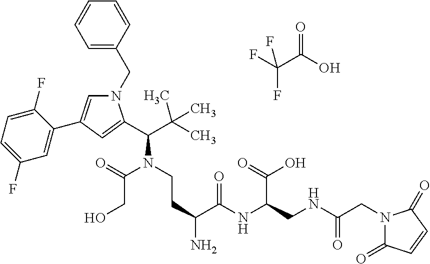 [SMILES: CC(C)(C)[C@H](c1cc(-c2cc(F)ccc2F)cn1Cc1ccccc1)N(CC[C@H](N)C(=O)N[C@H](CNC(=O)CN1C(=O)C=CC1=O)C(=O)O)C(=O)CO ; O=C(O)C(F)(F)F]

The synthesis of the title compound was carried out analogously to Intermediate FI 92 from 3-{[(benzyloxy)carbonyl]amino}-N-(tert-butoxycarbonyl)-D-alanine/N-cyclohexylcyclo-hexanamine (1:1).

LC-MS (Method 1): Rₜ=0.87 min; MS (ESIpos): m/z=737 (M+H)⁺.

### Intermediate F194

### N-{5-[(2,5-Dioxopyrrolidin-1-yl)oxy]-5-oxopentanoyl}-L-valyl-N-{3-[{(1R)-1-[1-benzyl-4-(2,5-difluorophenyl)-1H-pyrrol-2-yl]-2,2-dimethylpropyl}(glycoloyl)amino]propyl}-L- alaninamide

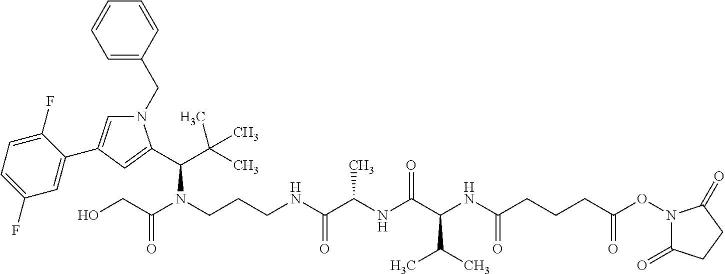 [SMILES: CC(C)[C@H](NC(=O)CCCC(=O)ON1C(=O)CCC1=O)C(=O)N[C@@H](C)C(=O)NCCCN(C(=O)CO)[C@@H](c1cc(-c2cc(F)ccc2F)cn1Cc1ccccc1)C(C)(C)C]

The title compound was prepared from Example M9 first by coupling with N-[(benzyloxy)carbonyl]-L-valyl-L-alanine in the presence of HATU and N,N-diisopropylethylamine. In the next step, the Z protective group was removed by hydrogenating for 1 hour over 10% palladium on activated carbon at RT under hydrogen standard pressure and then converting the deprotected intermediate by reaction with 1,1′-[(1,5-dioxopentane-1,5-diyl)bis(oxy)]dipyrrolidine-2,5-dione into the title compound.

LC-MS (Method 1): Rₜ=1.19 min; MS (ESIpos): m/z=851 [M+H]⁺.

### Intermediate F207

### N⁶—(N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-beta-alanyl)-N²—{N-[(2,5-dioxo-2,5-dihydro-1H-pyrrol-1-yl)acetyl]-L-valyl-L-alanyl}-L-lysine/trifluoroacetic acid (1:1)

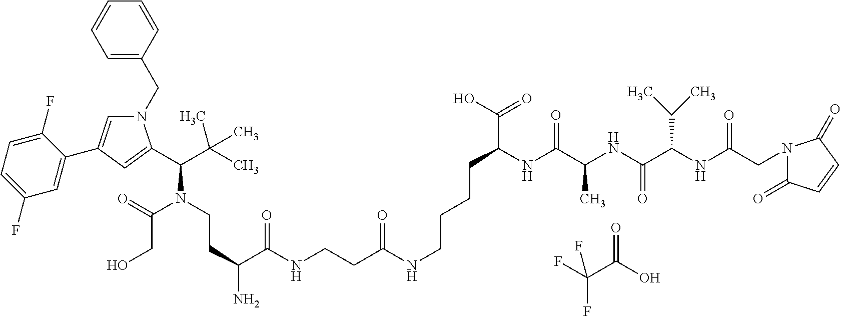

The title compound was prepared analogously to Intermediate F155.

LC-MS (Method 1): Rₜ=0.81 min; MS (ESIpos): m/z=1020 (M+H)⁺.

### Intermediate F216

### S-{2-[(3-Aminopropyl){(1R)-1-[1-benzyl-4-(2,5-difluorophenyl)-1H-pyrrol-2-yl]-2,2-dimethylpropyl}amino]-2-oxoethyl}-N-[19-(2,5-dioxo-2,5-dihydro-1H-pyrrol-1-yl)-17-oxo-4,7,10,13-tetraoxa-16-azanonadecan-1-oyl]-L-cysteinyl-beta-alanine/trifluoroacetic acid (1:1)

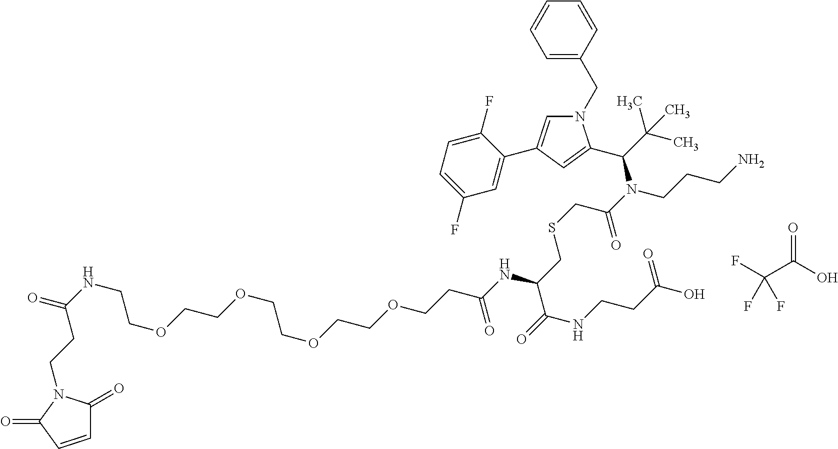 [SMILES: CC(C)(C)[C@H](c1cc(-c2cc(F)ccc2F)cn1Cc1ccccc1)N(CCCN)C(=O)CSC[C@H](NC(=O)CCOCCOCCOCCOCCNC(=O)CCN1C(=O)C=CC1=O)C(=O)NCCC(=O)O ; O=C(O)C(F)(F)F]

Under argon, 30.2 mg (0.06 mmol) of N,N′-bis[(benzyloxy)carbonyl]-L-cystine were initially charged in 2.0 ml of water and 2.0 ml of isopropanol, and 56.7 mg (0.20 mmol) of TCEP were added. The reaction mixture was stirred at RT for 30 min. 50.0 mg (0.08 mmol) of 2-(trimethylsilyl)ethyl {3-[{(1R)-1-[1-benzyl-4-(2,5-difluorophenyl)-1H-pyrrol-2-yl]-2,2-dimethylpropyl}(chloroacetyl)amino]propyl}carbamate (Intermediate C70), dissolved in 2.0 ml of isopropanol, and 122.2 mg (0.48 mmol) of 1,8-diazabicyclo[5.4.0]undec-7-ene were then added, and the reaction mixture was stirred at 50° C. for 7 h. Another 122.2 mg (0.48 mmol) of 1,8-diazabicyclo[5.4.0]undec-7-ene were then added, and the reaction mixture was stirred at 50° C. for 1 h. The mixture was diluted with ethyl acetate and the organic phase was extracted with water and saturated sodium bicarbonate solution and washed with saturated NaCl solution. The organic phase was dried over magnesium sulphate and the solvent was evaporated under reduced pressure. The residue was purified by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 43.1 mg (64% of theory) of the compound S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[(benzyloxy)carbonyl]-L-cysteine.

LC-MS (Method 1): Rₜ=1.46 min; MS (ESIpos): m/z=851 (M+H)⁺.

16.5 mg (0.05 mmol) of 4-methylbenzenesulphonic acid/benzyl beta-alaninate (1:1) were initially charged together with 14.0 mg (0.11 mmol) of N,N-diisopropylethylamine in 1.5 ml of acetonitrile. The reaction mixture was stirred at RT for 3 min, and 30.8 mg (0.04 mmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[(benzyloxy)carbonyl]-L-cysteine dissolved in 1.5 ml of acetonitrile, 23.4 mg (0.18 mmol) of N,N-diisopropylethylamine and 29.9 mg (0.05 mmol) of T3P (50% in ethyl acetate) were then added. The reaction mixture was stirred at RT overnight. Water was added, and the reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. The compound obtained was benzyl S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[(benzyloxy)carbonyl]-L-cysteinyl-beta-alaninate.

LC-MS (Method 1): Rₜ=1.59 min; MS (ESIpos): m/z=1012 (M+H)⁺.

43.8 mg (43.3 μmol) of benzyl S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[(benzyloxy)carbonyl]-L-cysteinyl-beta-alaninate were dissolved in 8.0 ml of ethanol, 4.4 mg of palladium on activated carbon (10%) were added and the mixture was hydrogenated at RT and standard pressure overnight. The reaction mixture was filtered through a cardboard filter and the filter cake was washed with ethanol. The solvent was evaporated under reduced pressure. Two more times, the residue was treated as just described. The residue was purified by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 14.5 mg (37% of theory) of the compound S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteinyl-beta-alanine/trifluoroacetic acid (1:1).

LC-MS (Method 1): Rₜ=1.08 min; MS (ESIpos): m/z=788 (M+H)⁺.

14.5 mg (16.1 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteinyl-beta-alanine/trifluoroacetic acid (1:1) were initially charged together with 9.1 mg (17.7 μmol) of 3-(2,5-dioxo-2,5-dihydro-1H-pyrrol-1-yl)-N-{15-[(2,5-dioxopyrrolidin-1-yl)oxy]-15-oxo-3,6,9,12-tetraoxapentadec-1-yl}propanamide in 1.0 ml of DMF, and 4.9 mg (48.2 μmol) of 4-methylmorpholine were added. The reaction mixture was stirred at RT overnight, and 3.4 mg (0.06 mmol) of acetic acid were then added. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 4.9 mg (50% of theory) of the compound S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteinyl-beta-alanine/trifluoroacetic acid (1:1).

LC-MS (Method 1): Rₜ=1.28 min; MS (ESIpos): m/z=1186 (M+H)⁺.

14.1 mg (11.9 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[19-(2,5-dioxo-2,5-dihydro-1H-pyrrol-1-yl)-17-oxo-4,7,10,13-tetraoxa-16-azanonadecan-1-oyl]-L-cysteinyl-beta-alanine/trifluoroacetic acid (1:1) were dissolved in 1.5 ml of trifluoroethanol, and 9.7 mg (71.3 μmol) of zinc dichloride were added. The reaction mixture was stirred at 50° C. for 3 h. Another 9.7 mg (71.3 μmol) of zinc dichloride were added and the reaction mixture was stirred at 50° C. for 3 h. Another 9.7 mg (71.3 μmol) of zinc dichloride were added and the reaction mixture was stirred at 70° C. for 4 h. 20.8 mg (0.07 mmol) of ethylenediamine-N,N,N′,N′-tetraacetic acid were added and the reaction mixture was stirred for 10 min, and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was lyophilized. This gave 6.2 mg (44% of theory) of the title compound.

LC-MS (Method 1): Rₜ=0.82 min; MS (ESIpos): m/z=1042 (M+H)⁺.

### Intermediate F239

### S-{2-[(3-Aminopropyl){(1R)-1-[1-benzyl-4-(2,5-difluorophenyl)-1H-pyrrol-2-yl]-2,2-dimethylpropyl}amino]-2-oxoethyl}-N-[(2,5-dioxo-2,5-dihydro-1H-pyrrol-1-yl)acetyl]-L-cysteine/trifluoroacetic acid (1:1)

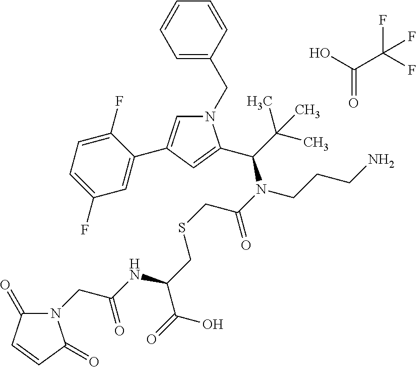 [SMILES: CC(C)(C)[C@H](c1cc(-c2cc(F)ccc2F)cn1Cc1ccccc1)N(CCCN)C(=O)CSC[C@H](NC(=O)CN1C(=O)C=CC1=O)C(=O)O ; O=C(O)C(F)(F)F]

Under argon, 7.5 mg (0.05 mmol) of (2,5-dioxo-2,5-dihydro-1H-pyrrol-1-ylacetic acid were initially charged in 1.5 ml of DMF, and 7.5 mg (0.05 mmol) of HOBt, 15.5 mg (0.05 mmol) of TBTU and 6.2 mg (0.05 mmol) of N,N-diisopropylethylamine were added. The reaction mixture was stirred at RT for 10 min. 40.0 mg (0.05 mmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine/trifluoroacetic acid (1:1) (Intermediate C71), dissolved in 1.5 ml of DMF, and 18.7 mg (0.14 mmol) of N,N-diisopropylethylamine were then added, and the reaction mixture was stirred at RT overnight. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 11.2 mg (25% of theory) of the compound S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[(2,5-dioxo-2,5-dihydro-1H-pyrrol-1-yl)acetyl]-L-cysteine.

LC-MS (Method 1): Rₜ=1.37 min; MS (ESIpos): m/z=854 (M+H)⁺.

10.9 mg (12.8 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[(2,5-dioxo-2,5-dihydro-1H-pyrrol-1-yl)acetyl]-L-cysteine were dissolved in 2.0 ml of trifluoroethanol, and 10.4 mg (76.6 μmol) zinc dichloride were added. The reaction mixture was stirred at 50° C. for 4 h. 22.4 mg (0.08 mmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 250×30; 10μ, flow rate: 50 ml/min, MeCN/water, 0.1% TFA). The solvents were evaporated under reduced pressure and the residue was lyophilized. This gave 7.5 mg (65% of theory) of the title compound.

LC-MS (Method 1): Rₜ=0.92 min; MS (ESIpos): m/z=710 (M+H)⁺.

### Intermediate F240

### Trifluoroacetic acid/3-({2-[(3-aminopropyl){(1R)-1-[1-benzyl-4-(2,5-difluorophenyl)-1H-pyrrol-2-yl]-2,2-dimethylpropyl}amino]-2-oxoethyl}sulphanyl)-N-(2-{[(2,5-dioxo-2,5-dihydro-1H-pyrrol-1-yl)acetyl]amino}ethylpropanamide (1:1)

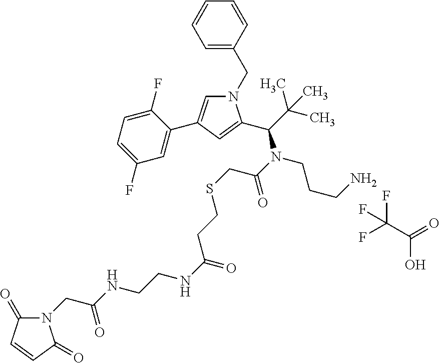

27.5 mg (0.04 mmol) of 11-{(1R)-1-[1-benzyl-4-(2,5-difluorophenyl)-1H-pyrrol-2-yl]-2,2-dimethylpropyl}-2,2-dimethyl-6,12-dioxo-5-oxa-14-thia-7,11-diaza-2-silaheptadecan-17-oic acid (Intermediate C69) were initially charged together with 15.9 mg (0.05 mmol) of trifluoroacetic acid/N-(2-aminoethyl)-2-(2,5-dioxo-2,5-dihydro-1H-pyrrol-1-ylacetamide (1:1) (Intermediate L1) in 1.8 ml of acetonitrile. 32.4 mg (0.31 mmol) of N,N-diisopropylethylamine were then added, and 32.4 mg (0.05 mmol) of T3P (50% in ethyl acetate) were added dropwise. The reaction mixture was stirred at RT overnight. The reaction mixture was purified directly by preparative RP-HPLC (column: Reprosil 125×30; 10μ, flow rate: 50 ml/min, MeCN/water). The solvents were evaporated under reduced pressure and the residue was dried under high vacuum. This gave 11.9 mg (35% of theory) of the compound 2-(trimethylsilyl)ethyl [13-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-2,7,12-trioxo-10-thia-3,6,13-triazahexadecan-16-yl]carbamate.

LC-MS (Method 1): Rₜ=1.39 min; MS (ESIpos): m/z=881 (M+H)⁺.

11.9 mg (0.01 mol) of 2-(trimethylsilyl)ethyl [13-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-2,7,12-trioxo-10-thia-3,6,13-triazahexadecan-16-yl]carbamate were dissolved in 1.0 ml of trifluoroethanol, and 5.5 mg (0.04 mmol) of zinc dichloride were added. The reaction mixture was stirred at 50° C. overnight. 11.8 mg (0.04 mmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 7.4 mg (60% of theory) of the title compound.

LC-MS (Method 5): Rₜ=2.75 min; MS (ESIpos): m/z=737 (M+H)⁺.

### Intermediate F241

### Trifluoroacetic acid/(2S)-2-amino-4-[{(1R)-1-[1-benzyl-4-(2,5-difluorophenyl)-1H-pyrrol-2-yl]-2,2-dimethylpropyl}(glycoloyl)amino]-N-(2-{[N-(bromoacetyl)glycyl]amino}ethylbutanamide (1:1)

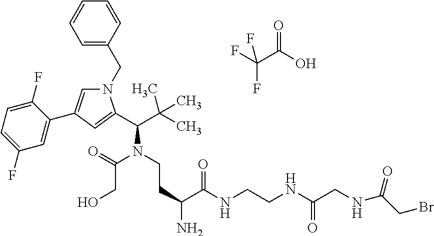 [SMILES: CC(C)(C)[C@H](c1cc(-c2cc(F)ccc2F)cn1Cc1ccccc1)N(CC[C@H](N)C(=O)NCCNC(=O)CNC(=O)CBr)C(=O)CO ; O=C(O)C(F)(F)F]

The title compound was prepared from Intermediate C66 by coupling with commercially available 1-(2-bromoacetoxy)pyrrolidine-2,5-dione and subsequent deblocking with zinc chloride.

LC-MS (Method 1): Rₜ=0.84 min; MS (EIpos): m/z=733 and 735 [M+H]⁺.

### Intermediate F242

### Trifluoroacetic acid/(2S)-2-amino-4-[{(1R)-1-[1-benzyl-4-(2,5-difluorophenyl)-1H-pyrrol-2-yl]-2,2-dimethylpropyl}(glycoloyl)amino]-N-(3-{[(2,5-dioxo-2,5-dihydro-1H-pyrrol-1-yl)acetyl]amino}propylbutanamide (1:1)

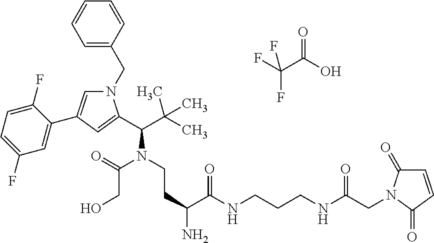 [SMILES: CC(C)(C)[C@H](c1cc(-c2cc(F)ccc2F)cn1Cc1ccccc1)N(CC[C@H](N)C(=O)NCCCNC(=O)CN1C(=O)C=CC1=O)C(=O)CO ; O=C(O)C(F)(F)F]

The synthesis of the title compound was carried out analogously to Intermediate F104.

LC-MS (Method 1): Rₜ=0.84 min; MS (ESIpos): m/z=707 (M+H)⁺.

### Intermediate F243

### Trifluoroacetic acid/(2S)-2-amino-4-[{(1R)-1-[1-benzyl-4-(2,5-difluorophenyl)-1H-pyrrol-2-yl]-2,2-dimethylpropyl}(glycoloyl)amino]-N-[2-(2-{[(2,5-dioxo-2,5-dihydro-1H-pyrrol-1-yl)acetyl]-amino}ethoxy)ethyl]butanamide (1:1)

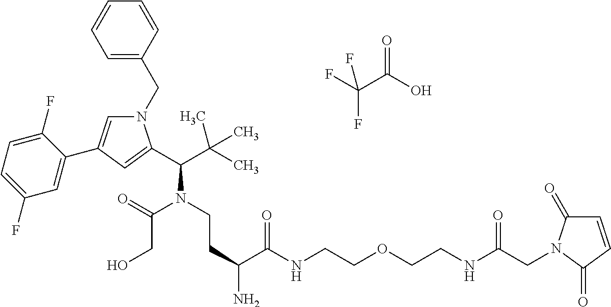 [SMILES: CC(C)(C)[C@H](c1cc(-c2cc(F)ccc2F)cn1Cc1ccccc1)N(CC[C@H](N)C(=O)NCCOCCNC(=O)CN1C(=O)C=CC1=O)C(=O)CO ; O=C(O)C(F)(F)F]

The synthesis of the title compound was carried out analogously to Intermediate F242.

LC-MS (Method 1): Rₜ=0.81 min; MS (ESIpos): m/z=737 (M+H)⁺.

### Intermediate F245

### Trifluoroacetic acid/N-{(2S)-2-amino-4-[{(1R)-1-[1-benzyl-4-(2,5-difluorophenyl)-1H-pyrrol-2-yl]-2,2-dimethylpropyl}(glycoloyl)amino]butyl}-N′-(2-{[(2,5-dioxo-2,5-dihydro-1H-pyrrol-1-yl-acetyl]amino}ethyl) succinamide (1:1)

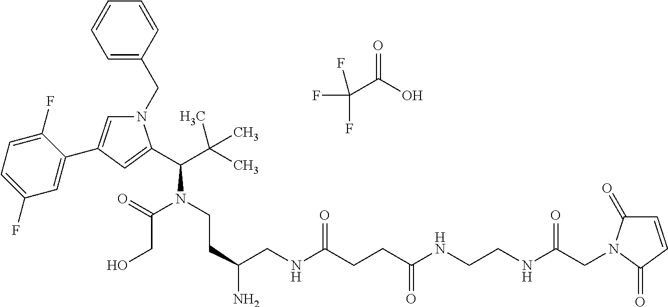 [SMILES: CC(C)(C)[C@H](c1cc(-c2cc(F)ccc2F)cn1Cc1ccccc1)N(CC[C@H](N)CNC(=O)CCC(=O)NCCNC(=O)CN1C(=O)C=CC1=O)C(=O)CO ; O=C(O)C(F)(F)F]

The title compound was prepared by coupling of 10 mg (0.0135 mmol) of Intermediate C65 with 8 mg (0.027 mmol) of Intermediate L1 in 8 ml of DMF in the presence of 15 mg (0.04 mmol) of HATU and 9 μl of N,N-diisopropylethylamine and subsequent deprotection with zinc chloride in trifluoroethanol as described for Intermediate F119. Purification by preparative HPLC gave 8.8 mg (58% of theory over 2 steps) of the title compound.

LC-MS (Method 1): Rₜ=0.84 min; MS (ESIpos): m/z=778 (M+H)⁺.

### Intermediate F247

### Trifluoroacetic acid/methyl 4-[(2-{[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)amino]-2-bromo-4-oxobutanoate (1:1)

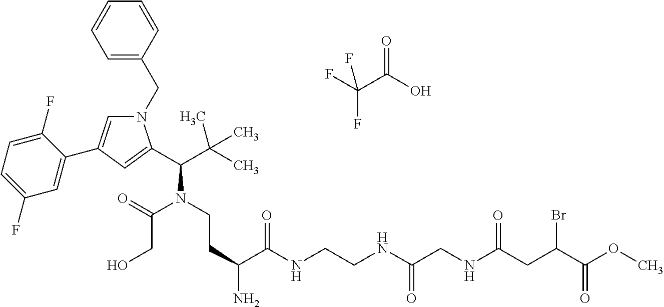 [SMILES: COC(=O)C(Br)CC(=O)NCC(=O)NCCNC(=O)[C@@H](N)CCN(C(=O)CO)[C@@H](c1cc(-c2cc(F)ccc2F)cn1Cc1ccccc1)C(C)(C)C ; O=C(O)C(F)(F)F]

14 mg (0.018 mmol) of Intermediate C66 were dissolved in 14 ml of DCM, and with 10.1 mg (0.037 mmol) of 2-bromo-1-ethylpyridinium tetrafluoroborate (BEP) and, a little at a time, a total of 250 μl of pyridine were added, the pH being kept between 5 and 6. The pH was then adjusted to 4 with acetic acid, the reaction was concentrated and the residue was purified by preparative HPLC. Combination of the appropriate fractions, lyophilization and drying gave 4 mg (21% of theory) of the protected intermediate, which were then deprotected at the amino function with zinc chloride. HPLC purification and lyophilization gave 3 mg (72% of theory) of the title compound as a colourless foam.

LC-MS (Method 1): Rₜ=0.88 min; MS (ESIpos): m/z=805 and 807 (M+H)⁺.

### Intermediate F248

### Trifluoroacetic acid/(2S)-2-amino-4-[{(1R)-1-[1-benzyl-4-(2,5-difluorophenyl)-1H-pyrrol-2-yl]-2,2-dimethylpropyl}(glycoloyl)amino]-N-{2-[2-(2,5-dioxo-2,5-dihydro-1H-pyrrol-1-yl)ethoxy]ethyl}butanamide (1:1)

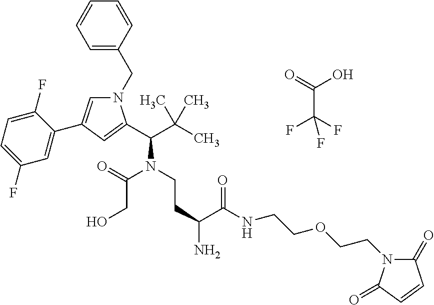 [SMILES: CC(C)(C)[C@H](c1cc(-c2cc(F)ccc2F)cn1Cc1ccccc1)N(CC[C@H](N)C(=O)NCCOCCN1C(=O)C=CC1=O)C(=O)CO ; O=C(O)C(F)(F)F]

The title compound was prepared by coupling of 10 mg (0.015 mmol) of Intermediate C58 with 5 mg (0.017 mmol) of Intermediate L12 in the presence of HATU and subsequent deprotection with zinc chloride. This gave 6.5 mg (52% of theory over 2 steps) of the title compound.

LC-MS (Method 1): Rₜ=0.91 min; MS (ESIpos): m/z=680 (M+H)⁺.

### Intermediate F254

### Trifluoroacetic acid/methyl (3S)-4-[(2-{[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]-amino}-2-oxoethyl)amino]-3-bromo-4-oxobutanoate (1:1)

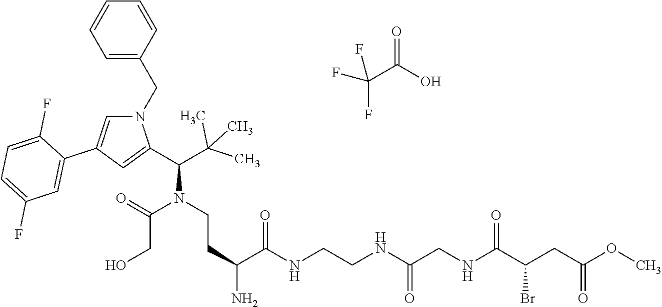 [SMILES: COC(=O)C[C@H](Br)C(=O)NCC(=O)NCCNC(=O)[C@@H](N)CCN(C(=O)CO)[C@@H](c1cc(-c2cc(F)ccc2F)cn1Cc1ccccc1)C(C)(C)C ; O=C(O)C(F)(F)F]

The title compound was prepared analogously to Intermediate 247 by coupling of 15 mg (0.02 mmol) of Intermediate C66 with 21 mg (0.099 mmol) of (2S)-2-bromo-4-methoxy-4-oxobutanoic acid which had been synthesized as described in (J. Org. Chem. 200, 65, 517-522) from (2S)-2-amino-4-methoxy-4-oxobutanoic acid hydrochloride (1:1).

LC-MS (Method 1): Rₜ=0.89 min; MS (ESIpos): m/z=805 and 807 (M+H)⁺.

### Intermediate F255

### R/S—(N-[19-(2,5-Dioxo-2,5-dihydro-1H-pyrrol-1-yl)-17-oxo-4,7,10,13-tetraoxa-16-azanonadecan-1-oyl]-L-alpha-glutamyl-S-{2-[(3-aminopropyl){(1R)-1-[1-benzyl-4-(2,5-difluorophenyl)-1H-pyrrol-2-yl]-2,2-dimethylpropyl}amino]-2-oxoethyl})homocysteine/trifluoroacetic acid (1:1)

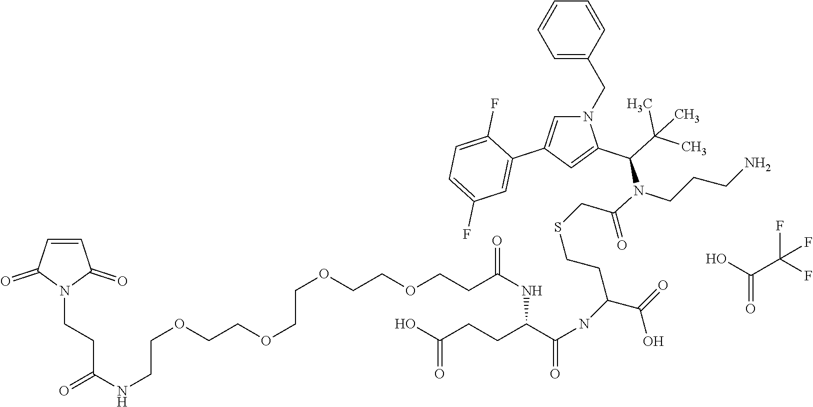 [SMILES: CC(C)(C)[C@H](c1cc(-c2cc(F)ccc2F)cn1Cc1ccccc1)N(CCCN)C(=O)CSCCC(NC(=O)[C@H](CCC(=O)O)NC(=O)CCOCCOCCOCCOCCNC(=O)CCN1C(=O)C=CC1=O)C(=O)O ; O=C(O)C(F)(F)F]

13.1 mg (0.04 mmol) of (2S)-5-(benzyloxy)-2-{[(benzyloxy)carbonyl]amino}-5-oxopentanoic acid were initially charged in 1.0 ml of DMF, and 5.4 mg (0.04 mmol) of HOBt, 11.4 mg (0.04 mmol) of TBTU and 4.6 mg (0.04 mmol) of N,N-diisopropylethylamine were added. The reaction mixture was stirred at RT for 10 min. 30.0 mg (0.04 mmol) of R/S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)homocysteine/trifluoroacetic acid (1:1) (Intermediate C11) dissolved in 12.9 mg (0.1 mmol) of N,N-diisopropylethylamine and 1 ml of DMF were then added. The reaction mixture was stirred at RT overnight. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 32 mg (73%) of the compound 4-[2-[[(1R)-1-[1-benzyl-4-(2,5-difluorophenyl)pyrrol-2-yl]-2,2-dimethylpropyl]-[3-(2-trimethylsilylethoxycarbonylamino)propyl]amino]-2-oxoethyl]sulphanyl-2-[[(2S)-5-benzyloxy-2-(benzyloxy)carbonylamino)-5-oxo-pentanoyl]amino]butanoic acid.

LC-MS (Method 1): Rₜ=1.53 min; MS (ESIpos): m/z=1084 (M+H)⁺.

41.4 mg (0.038 mmol) of 4-[2-[[(1R)-1-[1-benzyl-4-(2,5-difluorophenyl)pyrrol-2-yl]-2,2-dimethylpropyl]-[3-(2-trimethylsilylethoxycarbonylamino)propyl]amino]-2-oxoethyl]sulphanyl-2-[[(2S)-5-benzyloxy-2-(benzyloxy)carbonylamino)-5-oxo-pentanoyl]amino]butanoic acid was dissolved in 10 ml of ethanol, 4.2 mg of Pd/C were added and the mixture was hydrogenated under standard pressure. The reaction mixture was filtered through a cardboard filter and the filter cake was washed with ethanol. The solvent was evaporated under reduced pressure without heating. The residue was purified by preparative RP-HPLC (column: Reprosil 250×40; 10μ, flow rate: 50 ml/min, MeCN/water, 0.1% TFA). The solvents were evaporated under reduced pressure and the residue was dried under high vacuum. This gave 21.1 mg (56%) of the compound R/S-(L-alpha-glutamyl-S-(11-{(1R)-1-[1-benzyl-4-(2,5-difluorophenyl)-1H-pyrrol-2-yl]-2,2-dimethyl-propyl}-2,2-dimethyl-6,12-dioxo-5-oxa-7,11-diaza-2-silatridecan-13-yl)homocysteine/trifluoroacetic acid (1:1).

LC-MS (Method 1): Rₜ=1.11 min; MS (ESIpos): m/z=860 (M+H)⁺.

20.4 mg (20.94 μmol) of R/S-(L-alpha-glutamyl-S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl))homocysteine/trifluoroacetic acid (1:1) were initially charged together with 11.8 mg (23.04 μmol) of 3-(2,5-dioxo-2,5-dihydro-1H-pyrrol-1-yl)-N-{15-[(2,5-dioxopyrrolidin-1-yl)oxy]-15-oxo-3,6,9,12-tetraoxapentadec-1-yl}propanamide in 1.0 ml of DMF, and 4.2 mg (41.88 μmol) of 4-methylmorpholine were added. The reaction mixture was stirred at RT overnight, and 3.1 mg (0.05 mmol) of acetic acid were then added. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 9.5 mg (36%) of the compound R/S—(N-[19-(2,5-dioxo-2,5-dihydro-1H-pyrrol-1-yl)-17-oxo-4,7,10,13-tetraoxa-16-azanonadecan-1-oyl]-L-alpha-glutamyl-S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl) homocysteine.

LC-MS (Method 1): Rₜ=1.66 min; MS (ESIpos): m/z=1259 (M+H)⁺.

9.4 mg (7.47 μmol) of R/S—(N-[19-(2,5-dioxo-2,5-dihydro-1H-pyrrol-1-yl)-17-oxo-4,7,10,13-tetraoxa-16-azanonadecan-1-oyl]-L-alpha-glutamyl-S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl))homecysteine were dissolved in 1.5 ml of trifluoroethanol, and 6.1 mg (44.81 μmol) of zinc dichloride were added. The reaction mixture was stirred at 50° C. for 3 h. 13.1 mg (0.05 mmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 6.9 mg (75%) of the title compound.

LC-MS (Method 1): Rₜ=0.87 min; MS (ESIpos): m/z=1114 (M+H)⁺.

### Intermediate F256

### Trifluoroacetic acid/N-{(2S)-2-amino-4-[{(1R)-1-[1-benzyl-4-(2,5-difluorophenyl)-1H-pyrrol-2-yl]-2,2-dimethylpropyl}(glycoloyl)amino]butyl}-N′-[2-(2-{[(2,5-dioxo-2,5-dihydro-1H-pyrrol-1-yl) acetyl]amino}ethoxy)ethyl]succinamide (1:1)

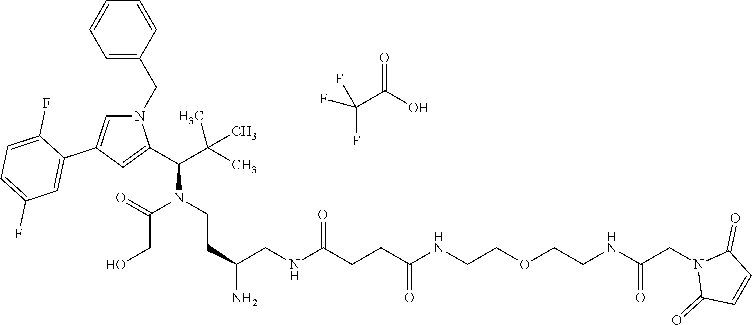 [SMILES: CC(C)(C)[C@H](c1cc(-c2cc(F)ccc2F)cn1Cc1ccccc1)N(CC[C@H](N)CNC(=O)CCC(=O)NCCOCCNC(=O)CN1C(=O)C=CC1=O)C(=O)CO ; O=C(O)C(F)(F)F]

The title compound was prepared by coupling of 10 mg (0.014 mmol) of Intermediate C65 and 9.6 mg (0.027 mmol) of trifluoroacetic acid/N-[2-(2-aminoethoxy)ethyl]-2-(2,5-dioxo-2,5-dihydro-1H-pyrrol-1-yl)acetamide (1:1) in the presence of HATU and N,N-diisopropylethylamine and subsequent deprotection with zinc chloride in trifluoroethanol as described for Intermediate F119. Purification by preparative HPLC gave 8 mg (64% of theory over 2 steps) of the title compound.

LC-MS (Method 1): Rₜ=0.84 min; MS (ESIpos): m/z=822 (M+H)⁺.

### Intermediate F257

### R-{2-[(3-Aminopropyl) {(1R)-1-[1-benzyl-4-(2,5-difluorophenyl)-1H-pyrrol-2-yl]-2,2-dimethylpropyl}amino]-2-oxoethyl}-N-[18-(2,5-dioxo-2,5-dihydro-1H-pyrrol-1-yl)-17-oxo-4,7,10,13-tetraoxa-16-azaoctadecan-1-oyl]-L-cysteine/trifluoroacetic acid (1:1)

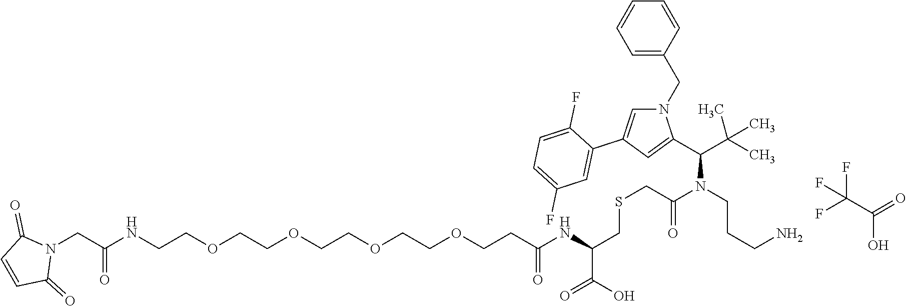

50.0 mg (0.06 mmol) of R-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine/trifluoroacetic acid (1:1) (Intermediate C71) and 29 mg (0.07 mmol) of 3-[2-[2-[2-[2-[[2-(2,5-dioxopyrrol-1-yl)acetyl]amino]ethoxy]ethoxy]ethoxy]ethoxy]propanoic acid (Intermediate L74) were dissolved in 3.0 ml of DMF, and 27.3 mg (0.07 mmol) of HATU and 23.3 mg (0.18 mmol) of N,N-diisopropylethylamine were added. The reaction mixture was stirred at RT for 2 hours. The reaction mixture was purified directly by preparative RP-HPLC (column: Reprosil 125×30; 10μ, flow rate: 50 ml/min, MeCN/water/0.1% TFA). The solvents were evaporated under reduced pressure and the residue was dried under high vacuum. This gave 17.4 mg (26%) of the compound R-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[18-(2,5-dioxo-2,5-dihydro-1H-pyrrol-1-yl)-17-oxo-4,7,10,13-tetraoxa-16-azaoctadecan-1-oyl]-L-cysteine.

LC-MS (Method 6): Rₜ=1.34 min; MS (ESIpos): m/z=1101 (M+H)⁺.

17 mg (0.02 mmol) of R-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[18-(2,5-dioxo-2,5-dihydro-1H-pyrrol-1-yl)-17-oxo-4,7,10,13-tetraoxa-16-azaoctadecan-1-oyl]-L-cysteine were dissolved in 1.0 ml of trifluoroethanol, and 6.3 mg (0.05 mmol) of zinc dichloride were added. The reaction mixture was stirred at 50° C. overnight. 13.5 mg (0.05 mmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 7.6 mg (46%) of the title compound.

LC-MS (Method 1): Rₜ=0.91 min; MS (ESIpos): m/z=957 (M+H)⁺.

### Intermediate F258

### Trifluoroacetic acid/(2S)-2-amino-4-[{(1R)-1-[1-benzyl-4-(2,5-difluorophenyl)-1H-pyrrol-2-yl]-2,2-dimethylpropyl}(glycoloyl)amino]-N-[3-{2-[(bromoacetyl)amino]ethyl}amino)-3-oxopropyl]butan amide (1:1)

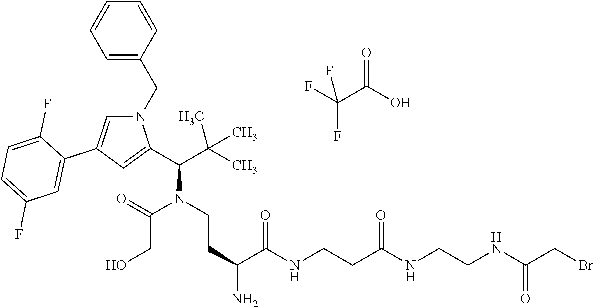 [SMILES: CC(C)(C)[C@H](c1cc(-c2cc(F)ccc2F)cn1Cc1ccccc1)N(CC[C@H](N)C(=O)NCCC(=O)NCCNC(=O)CBr)C(=O)CO ; O=C(O)C(F)(F)F]

The title compound was prepared by coupling of Intermediate C58 with trifluoroacetic acid/benzyl [2-(beta-alanylamino)ethyl]carbamate (1:1) using HATU, subsequent hydrogenolysis, followed by coupling with 1-(2-bromoacetoxy)pyrrolidine-2,5-dione and finally by deprotection with zinc chloride.

LC-MS (Method 1): Rₜ=0.86 min; MS (ESIpos): m/z=747 and 749 (M+H)⁺.

### Intermediate F259

### N-{(2S)-2-Amino-4-[{(1R)-1-[1-benzyl-4-(2,5-difluorophenyl)-1H-pyrrol-2-yl]-2,2-dimethyl propyl}(glycoloyl)amino]butanoyl}-3-{[N-(bromacetyl)glycyl]amino}-D-alanine/trifluoroacetic acid (1:1)

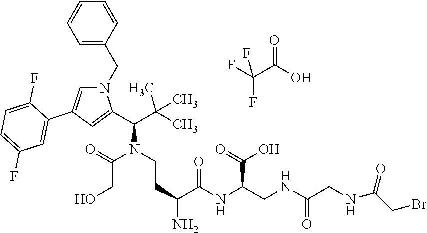 [SMILES: CC(C)(C)[C@H](c1cc(-c2cc(F)ccc2F)cn1Cc1ccccc1)N(CC[C@H](N)C(=O)N[C@H](CNC(=O)CNC(=O)CBr)C(=O)O)C(=O)CO ; O=C(O)C(F)(F)F]

75 mg (0.114 mmol) of Intermediate C58 were taken up in 12.5 ml of DMF and coupled with 78 mg (0.171 mmol) of Intermediate L75 in the presence of 65 mg (0.11 mmol) of HATU and 79 μl of N,N-diisopropylethylamine. After purification by preparative HPLC, the intermediate was taken up in 20 ml of ethanol and hydrogenated over 10% palladium on activated carbon at RT under hydrogen standard pressure for 1 h. The catalyst was then filtered off, the solvent was removed under reduced pressure and the product was purified by preparative HPLC. Lyophilization from acetonitrile/water 1:1 gave 63 mg (64% of theory over 2 steps) of 2-(trimethylsilyl)ethyl 3-amino-N-[(2S)-4-[{(1R)-1-[1-benzyl-4-(2,5-difluorophenyl)-1H-pyrrol-2-yl]-2,2-dimethylpropyl}(glycoloyl)amino]-2-({[2-(trimethylsilyl)ethoxy]carbonyl}aminobutanoyl]-D-alaninate.

LC-MS (Method 1): Rₜ=1.16 min; MS (EIpos): m/z=844 [M+H]⁺.

40 mg (0.047 mmol) of this intermediate were then coupled as described above with N-[(benzyloxy)carbonyl]glycine in the presence of HATU and then once more hydrogenolytically deprotected.

The title compound was then prepared by coupling of 10 mg (0.012 mmol) of this intermediate with 7.7 mg (0.032 mmol) of commercially available 1-(2-bromoacetoxy)pyrrolidine-2,5-dione in the presence of 4 μl of N,N-diisopropylethylamine and subsequent deprotection with zinc chloride in trifluoroethanol as described for Intermediate F119. Purification by preparative HPLC gave 1.3 mg of the title compound.

LC-MS (Method 1): Rₜ=0.83 min; MS (ESIpos): m/z=777 and 779 (M+H)⁺.

### Intermediate F260

### N⁶—(N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-beta-alanyl)-N²—{N-[(2,5-dioxo-2,5-dihydro-1H-pyrrol-1-yl)acetyl]-L-valyl-L-alanyl}-L-lysine/trifluoroacetic acid (1:1)

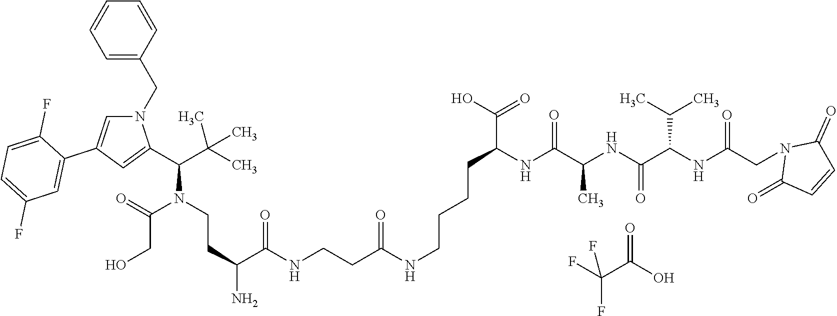

The title compound was prepared analogously to Intermediate F155.

LC-MS (Method 1): Rₜ=0.81 min; MS (ESIpos): m/z=1020 (M+H)⁺.

### Intermediate F261

### Trifluoroacetic acid/(2S)-2-amino-4-[{(1R)-1-[1-benzyl-4-(2,5-difluorophenyl)-1H-pyrrol-2-yl]-2,2-dimethylpropyl}(glycoloyl)amino]-N-(2-{2-[(bromoacetyl)amino]ethoxy}ethylbutanamide (1:1)

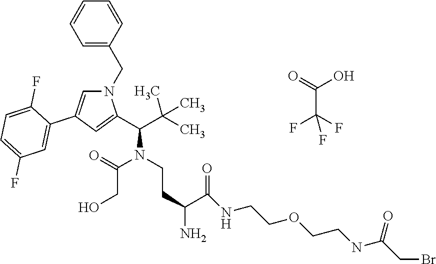 [SMILES: CC(C)(C)[C@H](c1cc(-c2cc(F)ccc2F)cn1Cc1ccccc1)N(CC[C@H](N)C(=O)NCCOCCNC(=O)CBr)C(=O)CO ; O=C(O)C(F)(F)F]

The title compound was prepared by coupling of 20 mg (0.03 mmol) of Intermediate C58 with 25.8 mg (0.061 mmol) of Intermediate L77 in the presence of HATU and subsequent deprotection with zinc chloride. This gave 11.9 mg (47% of theory over 2 steps) of the title compound.

LC-MS (Method 1): Rₜ=0.84 min; MS (ESIpos): m/z=722 and 720 (M+H)⁺.

### Intermediate F262

### S-{2-[(3-Aminopropyl){(1R)-1-[1-benzyl-4-(2,5-difluorophenyl)-1H-pyrrol-2-yl]-2,2-dimethylpropyl}amino]-2-oxoethyl}-N-{3-[2-(2-{[3-(2,5-dioxo-2,5-dihydro-1H-pyrrol-1-yl)propanoyl]amino}ethoxy)ethoxy]propanoyl}-L-cysteine/trifluoroacetic acid (1:1)

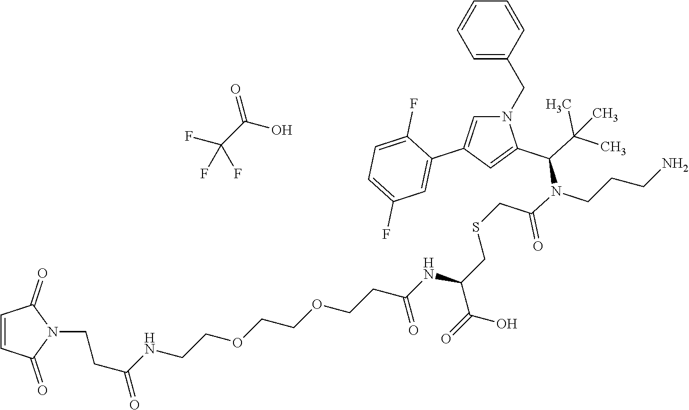 [SMILES: CC(C)(C)[C@H](c1cc(-c2cc(F)ccc2F)cn1Cc1ccccc1)N(CCCN)C(=O)CSC[C@H](NC(=O)CCOCCOCCNC(=O)CCN1C(=O)C=CC1=O)C(=O)O ; O=C(O)C(F)(F)F]

30 mg (36 μmol) of S-{2-[(3-aminopropyl){(1R)-1-[1-benzyl-4-(2,5-difluorophenyl)-1H-pyrrol-2-yl]-2,2-dimethylpropyl}amino]-2-oxoethyl}-L-cysteine/trifluoroacetic acid (1:1) (Intermediate C71) together with 16.9 mg (40 μmol) of 3-(2,5-dioxo-2,5-dihydro-1H-pyrrol-1-yl)-N-[2-(2-{3-[(2,5-dioxopyrrolidin-1-yl)oxy]-3-oxopropoxy}ethoxy)ethyl]propanamide were initially charged in 1.5 ml of DMF, and 10.9 mg (108 μmol) of 4-methylmorpholine were added. The reaction mixture was stirred at RT overnight, and 7.58 mg (0.13 mmol) of acetic acid were then added. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 33.4 mg (80% of theory) of the compound S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-{3-[2-(2-{[3-(2,5-dioxo-2,5-dihydro-1H-pyrrol-1-ylpropanoyl]amino}ethoxy)ethoxy]propanoyl}-L-cysteine.

LC-MS (Method 1): Rₜ=1.34 min; MS (ESIpos): m/z=1027 (M+H)⁺.

32.8 mg (32 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-{3-[2-(2-{[3-(2,5-dioxo-2,5-dihydro-1H-pyrrol-1-yl)propanoyl]amino}ethoxy)ethoxy]propanoyl}-L-cysteine were dissolved in 3.0 ml of trifluoroethanol, and 26.1 mg (192 μmol) of zinc dichloride were added. The reaction mixture was stirred at 50° C. for 2 h. 56.0 mg (0.192 mmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 250×30; 10μ, flow rate: 50 ml/min, MeCN/water, 0.1% TFA). The solvents were evaporated under reduced pressure and the residue was lyophilized. This gave 22.9 mg (71% of theory) of the title compound.

LC-MS (Method 1): Rₜ=0.88 min; MS (ESIpos): m/z=883 (M+H)⁺.

### Intermediate F263

### N-[(2,5-Dioxo-2,5-dihydro-1H-pyrrol-1-yl)acetyl]-beta-alanyl-S-{2-[(3-aminopropyl){(1R)-1-[1-benzyl-4-(2,5-difluorophenyl)-1H-pyrrol-2-yl]-2,2-dimethylpropyl}amino]-2-oxoethyl}-L-cysteine/trifluoroacetic acid (1:1)

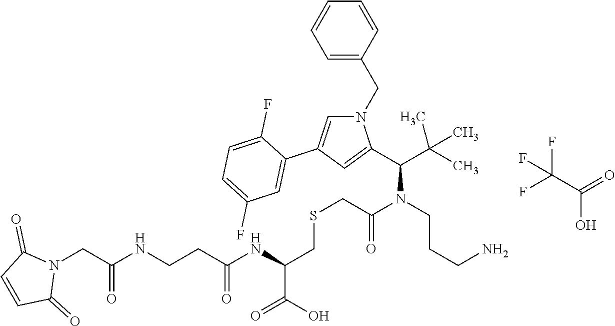 [SMILES: CC(C)(C)[C@H](c1cc(-c2cc(F)ccc2F)cn1Cc1ccccc1)N(CCCN)C(=O)CSC[C@H](NC(=O)CCNC(=O)CN1C(=O)C=CC1=O)C(=O)O ; O=C(O)C(F)(F)F]

30.0 mg (0.036 mmol) of R-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine/trifluoroacetic acid (1:1) (Intermediate C71) and 9.8 mg (0.04 mmol) of N-[(2,5-dioxo-2,5-dihydro-1H-pyrrol-1-ylacetyl]-beta-alanine (Intermediate L78) were dissolved in 1.0 ml of DMF, and 16.4 mg (0.04 mmol) of HATU and 14.0 mg (0.11 mmol) of N,N-diisopropylethylamine were added. The reaction mixture was stirred at RT for 2 hours. The reaction mixture was purified directly by preparative RP-HPLC (column: Reprosil 125×30; 10μ, flow rate: 50 ml/min, MeCN/water/0.1% TFA). The solvents were evaporated under reduced pressure and the residue was dried under high vacuum. This gave 4.2 mg (13%) of the compound N-[(2,5-dioxo-2,5-dihydro-1H-pyrrol-1-yl)acetyl]-beta-alanyl-S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine.

LC-MS (Method 6): Rₜ=1.31 min; MS (ESIpos): m/z=925 (M+H)⁺.

11.3 mg (0.011 mmol) of N-[(2,5-dioxo-2,5-dihydro-1H-pyrrol-1-ylacetyl]-beta-alanyl-S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine were dissolved in 2.0 ml of trifluoroethanol, and 5.0 mg (0.04 mmol) of zinc dichloride were added. The reaction mixture was stirred at 50° C. for 2 hours. 10.7 mg (0.04 mmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 4.4 mg (40%) of the title compound.

LC-MS (Method 1): Rₜ=0.91 min; MS (ESIpos): m/z=781 (M+H)⁺.

### Intermediate F264

### N-[6-(2,5-Dioxo-2,5-dihydro-1H-pyrrol-1-yl)hexanoyl]-beta-alanyl-S-{2-[(3-aminopropyl){(1R)-1-[1-benzyl-4-(2,5-difluorophenyl)-1H-pyrrol-2-yl]-2,2-dimethylpropyl}amino]-2-oxoethyl}-L-cysteine/trifluoroacetic acid (1:1)

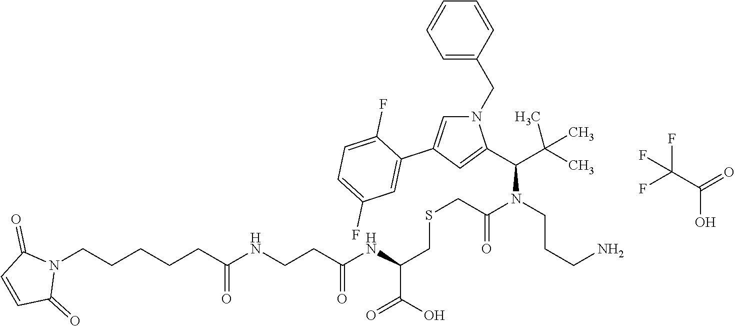 [SMILES: CC(C)(C)[C@H](c1cc(-c2cc(F)ccc2F)cn1Cc1ccccc1)N(CCCN)C(=O)CSC[C@H](NC(=O)CCNC(=O)CCCCCN1C(=O)C=CC1=O)C(=O)O ; O=C(O)C(F)(F)F]

30.0 mg (0.036 mmol) of R-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine/trifluoroacetic acid (1:1) (Intermediate C71) and 12.2 mg (0.04 mmol) of N-[6-(2,5-dioxo-2,5-dihydro-1H-pyrrol-1-yl)hexanoyl]-beta-alanine (Intermediate L79) were dissolved in 1.0 ml of DMF, and 16.4 mg (0.04 mmol) of HATU and 14.0 mg (0.11 mmol) of N,N-diisopropylethylamine were added. The reaction mixture was stirred at RT for 2 hours. The reaction mixture was purified directly by preparative RP-HPLC (column: Reprosil 125×30; 10μ, flow rate: 50 ml/min, MeCN/water/0.1% TFA). The solvents were evaporated under reduced pressure and the residue was dried under high vacuum. This gave 8.9 mg (24%) of the compound N-[6-(2,5-dioxo-2,5-dihydro-1H-pyrrol-1-yl)hexanoyl]-beta-alanyl-S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine.

LC-MS (Method 6): Rₜ=1.38 min; MS (ESIpos): m/z=981 (M+H)⁺.

15.3 mg (0.015 mmol) of N-[6-(2,5-dioxo-2,5-dihydro-1H-pyrrol-1-yl)hexanoyl]-beta-alanyl-S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine were dissolved in 2.0 ml of trifluoroethanol, and 6.3 mg (0.045 mmol) of zinc dichloride were added. The reaction mixture was stirred at 50° C. for 2 hours. 13.5 mg (0.045 mmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 9.1 mg (62%) of the title compound.

LC-MS (Method 1): Rₜ=0.92 min; MS (ESIpos): m/z=837 (M+H)⁺.

### Intermediate F265

### Trifluoroacetic acid/N-(3-aminopropyl)-N-{(1R)-1-[1-benzyl-4-(2,5-difluorophenyl)-1H-pyrrol-2-yl]-2,2-dimethylpropyl}-22-(2,5-dioxo-2,5-dihydro-1H-pyrrol-1-yl)-6,17-dioxo-10,13-dioxa-3-thia-7,16-diazadocosane-1-amide (1:1)

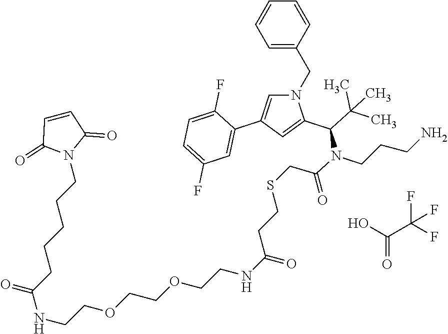 [SMILES: CC(C)(C)[C@H](c1cc(-c2cc(F)ccc2F)cn1Cc1ccccc1)N(CCCN)C(=O)CSCCC(=O)NCCOCCOCCNC(=O)CCCCCN1C(=O)C=CC1=O ; O=C(O)C(F)(F)F]

30.0 mg (42.7 μmol) of 11-{(1R)-1-[1-benzyl-4-(2,5-difluorophenyl)-1H-pyrrol-2-yl]-2,2-dimethylpropyl}-2,2-dimethyl-6,12-dioxo-5-oxa-14-thia-7,11-diaza-2-silaheptadecan-17-oic acid (Intermediate C69) and 25.3 mg (55.6 μmol) of trifluoroacetic acid/N-{2-[2-(2-aminoethoxy)ethoxy]ethyl}-6-(2,5-dioxo-2,5-dihydro-1H-pyrrol-1-ylhexanamide (1:1) (Intermediate L82) were initially charged in 1.9 ml of acetonitrile, and 60 μl (340 μmol) of N,N-diisopropylethylamine and 33 μl (56 μmol) of 2,4,6-tripropyl-1,3,5,2,4,6-trioxatriphosphinane 2,4,6-trioxide 50% in ethyl acetate were added. The reaction mixture was stirred at RT overnight. Water (2.0 ml) was added, and purification was carried out directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 26.7 mg (60% of theory) of the compound 2-(trimethylsilyl)ethyl [4-{(1R)-1-[1-benzyl-4-(2,5-difluorophenyl)-1H-pyrrol-2-yl]-2,2-dimethylpropyl}-26-(2,5-dioxo-2,5-dihydro-1H-pyrrol-1-yl)-5,10,21-trioxo-14,17-dioxa-7-thia-4,11,20-triazahexacos-1-yl]carbamate.

LC-MS (Method 1): Rₜ=1.40 min; MS (ESIpos): m/z=1025 (M+H)⁺.

25.3 mg (24.7 μmol) of 2-(trimethylsilyl)ethyl [4-{(1R)-1-[1-benzyl-4-(2,5-difluorophenyl)-1H-pyrrol-2-yl]-2,2-dimethylpropyl}-26-(2,5-dioxo-2,5-dihydro-1H-pyrrol-1-yl)-5,10,21-trioxo-14,17-dioxa-7-thia-4,11,20-triazahexacos-1-yl]carbamate were dissolved in 2.0 ml of trifluoroethanol, and 20.2 mg (148 μmol) of zinc dichloride were added. The reaction mixture was stirred at 50° C. for 1 h. 43.3 mg (148 μmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 23.4 mg (95% of theory) of the title compound.

LC-MS (Method 1): Rₜ=0.89 min; MS (ESIpos): m/z=881 (M+H)⁺.

### Intermediate F266

### Trifluoroacetic acid/N-(3-aminopropyl)-N-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-2,13-dioxo-6,9-dioxa-16-thia-3,12-diazaoctadecan-18-amide (1:1)

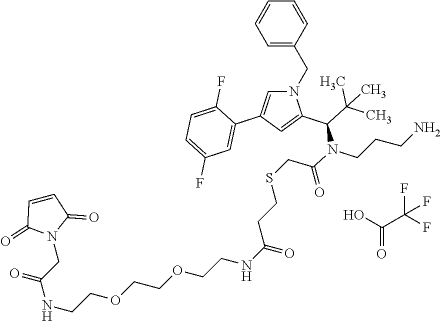 [SMILES: CC(C)(C)[C@H](c1cc(-c2cc(F)ccc2F)cn1Cc1ccccc1)N(CCCN)C(=O)CSCCC(=O)NCCOCCOCCNC(=O)CN1C(=O)C=CC1=O ; O=C(O)C(F)(F)F]

30.0 mg (0.043 mmol) of 11-{(1R)-1-[1-benzyl-4-(2,5-difluorophenyl)-1H-pyrrol-2-yl]-2,2-dimethylpropyl}-2,2-dimethyl-6,12-dioxo-5-oxa-14-thia-7,11-diaza-2-silaheptadecan-17-oic acid (Intermediate C69) were initially charged together with 22.2 mg (0.056 mmol) of trifluoroacetic acid/N-{2-[2-(2-aminoethoxy)ethoxy]ethyl}-2-(2,5-dioxo-2,5-dihydro-1H-pyrrol-1-ylacetamide (1:1) (Intermediate L83) in 1.9 ml of acetonitrile. 60 μl (0.34 mmol) of N,N-diisopropylethylamine were then added, and 33 μl (0.056 mmol) of T3P (50% in ethyl acetate) were added dropwise. The reaction mixture was stirred at RT overnight. Water (2.0 ml) was added. The reaction mixture was purified directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 20.5 mg (49% of theory) of the compound 2-(trimethylsilyl)ethyl [19-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-2,13,18-trioxo-6,9-dioxa-16-thia-3,12,19-triazadocosan-22-yl]carbamate.

LC-MS (Method 1): Rₜ=1.38 min; MS (ESIpos): m/z=969 (M+H)⁺.

19.1 mg (19.7 μmol) of 2-(trimethylsilyl)ethyl [19-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-2,13,18-trioxo-6,9-dioxa-16-thia-3,12,19-triazadocosan-22-yl]carbamate were dissolved in 2.0 ml of trifluoroethanol, and 16.1 mg (118 μmol) of zinc dichloride were added. The reaction mixture was stirred at 50° C. for 1 h. 34.6 mg (118 μmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 13.9 mg (75% of theory) of the title compound.

LC-MS (Method 1): Rₜ=0.86 min; MS (ESIpos): m/z=825 (M+H)⁺.

### Intermediate F267

### S-{2-[(3-Aminopropyl){(1R)-1-[1-benzyl-4-(2,5-difluorophenyl)-1H-pyrrol-2-yl]-2,2-dimethylpropyl}amino]-2-oxoethyl}-N-[1-(2,5-dioxo-2,5-dihydro-1H-pyrrol-1-yl)-2,18-dioxo-6,9,12,15-tetraoxa-3-azaoctadecan-18-yl]-L-cysteinyl-beta-alanine/trifluoroacetic acid (1:1)

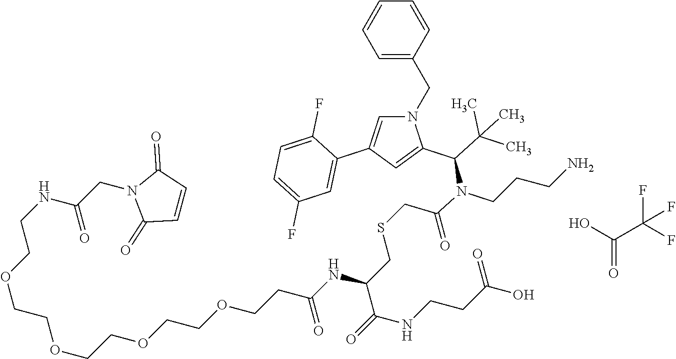 [SMILES: CC(C)(C)[C@H](c1cc(-c2cc(F)ccc2F)cn1Cc1ccccc1)N(CCCN)C(=O)CSC[C@H](NC(=O)CCOCCOCCOCCOCCNC(=O)CN1C(=O)C=CC1=O)C(=O)NCCC(=O)O ; O=C(O)C(F)(F)F]

Under argon, 13.4 mg (33.3 μmol) of 1-(2,5-dioxo-2,5-dihydro-1H-pyrrol-1-yl)-2-oxo-6,9,12,15-tetraoxa-3-azaoctadecan-18-oic acid (Intermediate L74) were initially charged in 1.0 ml of DMF, and 9.3 μl (54.4 μmol) of N,N-diisopropylethylamine and 12.6 mg (33.3 μmol) of HATU were added. The reaction mixture was stirred at RT for 10 min. 25.0 mg (27.7 μmol) of S-(11-{(1R)-1-[l-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteinyl-beta-alanine/trifluoroacetic acid (1:1) (see synthesis of Intermediate F216) dissolved in 4.7 μl (27.7 μmol) of N,N-diisopropylethy 1 amine and 1.0 ml of DMF were then added. The reaction mixture was stirred at RT for 90 minutes. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 6.90 mg (19% of theory) of the compound S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[1-(2,5-dioxo-2,5-dihydro-1H-pyrrol-1-yl)-2,18-dioxo-6,9,12,15-tetraoxa-3-azaoctadecan-18-yl]-L-cysteinyl-beta-alanine.

LC-MS (Method 5): Rₜ=4.44 min; MS (ESIpos): m/z=1172 (M+H)⁺.

6.70 mg (5.71 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[1-(2,5-dioxo-2,5-dihydro-1H-pyrrol-1-yl)-2,18-dioxo-6,9,12,15-tetraoxa-3-azaoctadecan-18-yl]-L-cysteinyl-beta-alanine were dissolved in 1.0 ml of trifluoroethanol, and 4.67 mg (34.3 μmol) of zinc dichloride were added. The reaction mixture was stirred at 50° C. for 1 h. 10 mg (34.3 μmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 4.4 mg (67% of theory) of the title compound.

LC-MS (Method 1): Rₜ=0.85 min; MS (ESIpos): m/z=1028 (M+H)⁺.

### Intermediate F268

### Trifluoroacetic acid/N-(3-aminopropyl)-N-{(1R)-1-[1-benzyl-4-(2,5-difluorophenyl)-1H-pyrrol-2-yl]-2,2-dimethylpropyl}-28-(2,5-dioxo-2,5-dihydro-1H-pyrrol-1-yl)-6,23-dioxo-10,13,16,19-tetraoxa-3-thia-7,22-diazaoctacosane-1-amide (1:1)

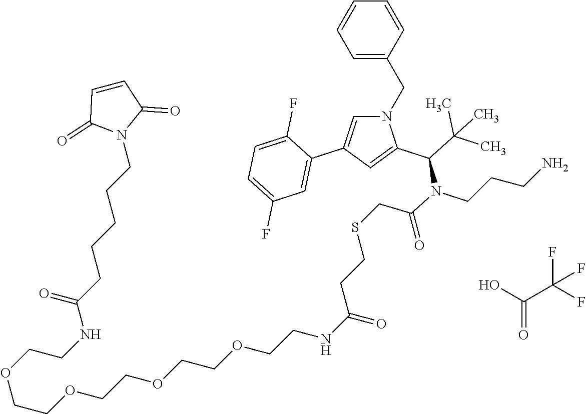 [SMILES: CC(C)(C)[C@H](c1cc(-c2cc(F)ccc2F)cn1Cc1ccccc1)N(CCCN)C(=O)CSCCC(=O)NCCOCCOCCOCCOCCNC(=O)CCCCCN1C(=O)C=CC1=O ; O=C(O)C(F)(F)F]

30.0 mg (0.043 mmol) of 11-{(1R)-1-[1-benzyl-4-(2,5-difluorophenyl)-1H-pyrrol-2-yl]-2,2-dimethylpropyl}-2,2-dimethyl-6,12-dioxo-5-oxa-14-thia-7,11-diaza-2-silaheptadecan-17-oic acid (Intermediate C69) were initially charged together with 30.2 mg (0.056 mmol) of trifluoroacetic acid/N-(14-amino-3,6,9,12-tetraoxatetradec-1-yl)-6-(2,5-dioxo-2,5-dihydro-1H-pyrrol-1-ylhexanamide (1:1) (Intermediate L84) in 2.0 ml of acetonitrile. 60 μl (0.34 mmol) of N,N-diisopropylethylamine were then added, and 33 μl (0.056 mmol) of T3P (50% in ethyl acetate) were added dropwise. The reaction mixture was stirred at RT overnight. Water (2.0 ml) was added. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 27.9 mg (59% of theory) of the compound 2-(trimethylsilyl)ethyl [4-{(1R)-1-[1-benzyl-4-(2,5-difluorophenyl)-1H-pyrrol-2-yl]-2,2-dimethylpropyl}-32-(2,5-dioxo-2,5-dihydro-1H-pyrrol-1-yl)-5,10,27-trioxo-14,17,20,23-tetraoxa-7-thia-4,11,26-triazadotriacont-1-yl]carbamate.

LC-MS (Method 1): Rₜ=1.41 min; MS (ESIpos): m/z=1114 (M+H)⁺.

25.6 mg (23.0 μmol) of 2-(trimethylsilyl)ethyl [4-{(1R)-1-[1-benzyl-4-(2,5-difluorophenyl)-1H-pyrrol-2-yl]-2,2-dimethylpropyl}-32-(2,5-dioxo-2,5-dihydro-1H-pyrrol-1-yl)-5,10,27-trioxotrioxo-14,17,20,23-tetraoxa-7-thia-4,11,26-triazadotriacont-1-yl]carbamate were dissolved in 2.5 ml of trifluoroethanol, and 18.8 mg (138 μmol) of zinc dichloride were added. The reaction mixture was stirred at 50° C. for 1 h. 40.3 mg (138 μmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 22.2 mg (88% of theory) of the title compound.

LC-MS (Method 1): Rₜ=0.94 min; MS (ESIpos): m/z=969 (M+H)⁺.

### Intermediate F269

### 4-{[(8R,14R)-13-(3-Aminopropyl)-14-[1-benzyl-4-(2,5-difluorophenyl)-1H-pyrrol-2-yl]-1-(2,5-dioxo-2,5-dihydro-1H-pyrrol-1-yl)-15,15-dimethyl-2,7,12-trioxo-10-thia-3,6,13-triazahexadecan-8-yl]amino}-4-oxobutanoic acid/trifluoroacetic acid (1:1)

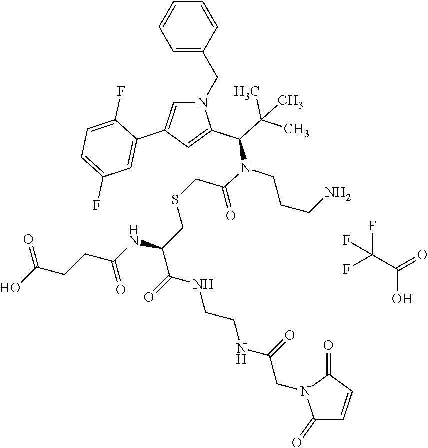 [SMILES: CC(C)(C)[C@H](c1cc(-c2cc(F)ccc2F)cn1Cc1ccccc1)N(CCCN)C(=O)CSC[C@H](NC(=O)CCC(=O)O)C(=O)NCCNC(=O)CN1C(=O)C=CC1=O ; O=C(O)C(F)(F)F]

17.0 mg (0.0195 mmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-(4-tert-butoxy-4-oxobutanoyl)-L-cysteine (Intermediate C77) were initially charged together with 4.99 mg (0.0253 mmol) of N-(2-aminoethyl)-2-(2,5-dioxo-2,5-dihydro-1H-pyrrol-1-yl)acetamide (Intermediate L1) in 1.0 ml of acetonitrile. 27 μl (0.16 mmol) of N,N-diisopropylethylamine were then added, and 15 μl (0.025 mmol) of T3P (50% in ethyl acetate) were added dropwise. The reaction mixture was stirred at RT overnight. Water (2.0 ml) was added. The reaction mixture was purified directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 9.5 mg (46% of theory) of the compound tert-butyl 4-{[(16R)-11-{(1R)-1-[1-benzyl-4-(2,5-difluorophenyl)-1H-pyrrol-2-yl]-2,2-dimethylpropyl}-23-(2,5-dioxo-2,5-dihydro-1H-pyrrol-1-yl)-2,2-dimethyl-6,12,17,22-tetraoxo-5-oxa-14-thia-7,11,18,21-tetraaza-2-silatricosan-16-yl]amino}-4-oxobutanoate.

LC-MS (Method 1): Rₜ=1.47 min; MS (ESIpos): m/z=1052 (M+H)⁺.

8.3 mg (7.89 μmol) of tert-butyl 4-{[(16R)-11-{(1R)-1-[1-benzyl-4-(2,5-difluorophenyl)-1H-pyrrol-2-yl]-2,2-dimethylpropyl}-23-(2,5-dioxo-2,5-dihydro-1H-pyrrol-1-yl)-2,2-dimethyl-6,12,17,22-tetraoxo-5-oxa-14-thia-7,11,18,21-tetraaza-2-silatricosan-16-yl]amino}-4-oxobutanoate were dissolved in 1.0 ml of trifluoroethanol, and 6.45 mg (47.3 μmol) of zinc dichloride were added. The reaction mixture was stirred at 50° C. for 6 h. 6.45 mg (47.3 μmol) of zinc dichloride were added and the reaction mixture was stirred at 50° C. overnight. 27.7 mg (94.6 μmol) of ethylenediamine-N,N,N′,N′-tetraacetic acid were added and the reaction mixture was stirred for 10 min, and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 1.10 mg (14% of theory) of the title compound.

LC-MS (Method 1): Rₜ=0.89 min; MS (ESIpos): m/z=852 (M+H)⁺.

### Intermediate F270

### Trifluoroacetic acid/N-(3-aminopropyl)-N-{(1R)-1-[1-benzyl-4-(2,5-difluorophenyl)-1H-pyrrol-2-yl]-2,2-dimethylpropyl}-N′-(2-{[(2,5-dioxo-2,5-dihydro-1H-pyrrol-1-yl) acetyl]amino}ethyl) succinamide (1:1)

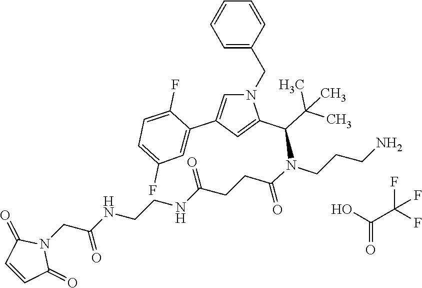 [SMILES: CC(C)(C)[C@H](c1cc(-c2cc(F)ccc2F)cn1Cc1ccccc1)N(CCCN)C(=O)CCC(=O)NCCNC(=O)CN1C(=O)C=CC1=O ; O=C(O)C(F)(F)F]

Under argon, 15.0 mg (22.9 μmol) of 11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silapentadecan-15-oic acid (Intermediate C78) were initially charged in 1.0 ml of DMF, and 8.0 μl (45.8 μmol) of N,N-diisopropylethylamine and 10.4 mg (27.4 μmol) of HATU were added. The reaction mixture was stirred at RT for 10 min. 8.54 mg (27.4 μmol) of trifluoroacetic acid/N-(2-aminoethyl)-2-(2,5-dioxo-2,5-dihydro-1H-pyrrol-1-yl)acetamide (1:1) (Intermediate L1) dissolved in 4.0 μl (22.9 μmol) of N,N-diisopropylethylamine and 1.0 ml of DMF were then added. The reaction mixture was stirred at RT for 1 h. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 14.7 mg (77% of theory) of the compound 2-(trimethylsilyl)ethyl [3-({(1R)-1-[1-benzyl-4-(2,5-difluorophenyl)-1H-pyrrol-2-yl]-2,2-dimethylpropyl}{4-[(2-{[(2,5-dioxo-2,5-dihydro-1H-pyrrol-1-yl)acetyl]amino}ethyl)amino]-4-oxobutanoyl}amino)propyl]carbamate.

LC-MS (Method 5): Rₜ=1.33 min; MS (ESIpos): m/z=835 (M+H)⁺.

13.2 mg (15.8 μmol) of 2-(trimethylsilyl)ethyl [3-({(1R)-1-[1-benzyl-4-(2,5-difluorophenyl)-1H-pyrrol-2-yl]-2,2-dimethylpropyl}{4-[(2-{[(2,5-dioxo-2,5-dihydro-1H-pyrrol-1-ylacetyl]amino}ethyl)amino]-4-oxobutanoyl}amino)propyl]carbamate were dissolved in 2.0 ml of trifluoroethanol, and 12.9 mg (94.8 μmol) of zinc dichloride were added. The reaction mixture was stirred at 50° C. for 1 h. 27.7 mg (94.6 μmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 10.9 mg (83% of theory) of the title compound.

LC-MS (Method 1): Rₜ=0.83 min; MS (ESIpos): m/z=691 (M+H)⁺.

### Intermediate F271

### 4-{[(20R,26R)-25-(3-Aminopropyl)-26-[1-benzyl-4-(2,5-difluorophenyl)-1H-pyrrol-2-yl]-1-(2,5-dioxo-2,5-dihydro-1H-pyrrol-1-yl)-27,27-dimethyl-2,19,24-trioxo-6,9,12,15-tetraoxa-22-thia-3,18,25-triazaoctacosan-20-yl]amino}-4-oxobutanoic acid/trifluoroacetic acid (1:1)

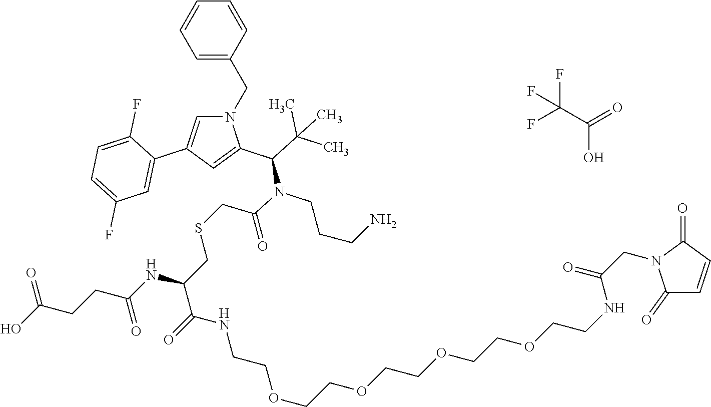 [SMILES: CC(C)(C)[C@H](c1cc(-c2cc(F)ccc2F)cn1Cc1ccccc1)N(CCCN)C(=O)CSC[C@H](NC(=O)CCC(=O)O)C(=O)NCCOCCOCCOCCOCCNC(=O)CN1C(=O)C=CC1=O ; O=C(O)C(F)(F)F]

Under argon, 19.4 mg (22.2 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-(4-tert-butoxy-4-oxobutanoyl)-L-cysteine (Intermediate C77) were initially charged in 2.0 ml of DMF, and 21.7 mg (44.4 μmol) of trifluoroacetic acid/N-(14-amino-3,6,9,12-tetraoxatetradec-1-yl)-2-(2,5-dioxo-2,5-dihydro-1H-pyrrol-1-yl)acetamide (1:1) (Intermediate L74), 12 μl (67 μmol) of N,N-diisopropylethylamine and 16.9 mg (44.4 μmol) of HATU were added. The reaction mixture was stirred at RT for 1 h. The reaction mixture was purified directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 18.1 mg (66% of theory) of the compound tert-butyl 4-{[(16R)-11-{(1R)-1-[1-benzyl-4-(2,5-difluorophenyl)-1H-pyrrol-2-yl]-2,2-dimethylpropyl}-35-(2,5-dioxo-2,5-dihydro-1H-pyrrol-1-yl)-2,2-dimethyl-6,12,17,34-tetraoxo-5,21,24,27,30-pentaoxa-14-thia-7,11,18,33-tetraaza-2-silapentatriacontan-16-yl]amino}-4-oxobutanoate.

LC-MS (Method 4): Rₜ=1.79 min; MS (ESIpos): m/z=1250 (M+Na)⁺.

18.1 mg (14.7 μmol) of tert-butyl 4-{[(16R)-11-{(1R)-1-[1-benzyl-4-(2,5-difluorophenyl)-1H-pyrrol-2-yl]-2,2-dimethylpropyl}-35-(2,5-dioxo-2,5-dihydro-1H-pyrrol-1-yl)-2,2-dimethyl-6,12,17,34-tetraoxo-5,21,24,27,30-pentaoxa-14-thia-7,11,18,33-tetraaza-2-silapentatriacontan-16-yl]amino}-4-oxobutanoate were dissolved in 2.0 ml of trifluoroethanol, and 12.0 mg (88.4 μmol) of zinc dichloride were added. The reaction mixture was stirred at 50° C. for 4 h. 25.8 mg (88.4 μmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 12.3 mg (73% of theory) of the title compound.

LC-MS (Method 1): Rₜ=0.87 min; MS (ESIpos): m/z=1028 (M+H)⁺.

### Intermediate F272

### Trifluoroacetic acid/N-(3-aminopropyl)-N-{(1R)-1-[1-benzyl-4-(2,5-difluorophenyl)-1H-pyrrol-2-yl]-2,2-dimethylpropyl}-N′-[17-(2,5-dioxo-2,5-dihydro-1H-pyrrol-1-yl)-16-oxo-3,6,9,12-tetraoxa-15-azaheptadec-1-yl]succinamide (1:1)

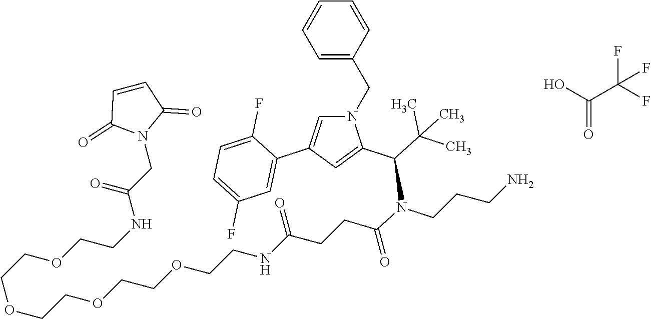 [SMILES: CC(C)(C)[C@H](c1cc(-c2cc(F)ccc2F)cn1Cc1ccccc1)N(CCCN)C(=O)CCC(=O)NCCOCCOCCOCCOCCNC(=O)CN1C(=O)C=CC1=O ; O=C(O)C(F)(F)F]

Under argon, 15.0 mg (22.9 μmol) of 11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silapentadecan-15-oic acid (Intermediate C78) were initially charged in 1.0 ml of DMF, and 8.0 μl (45.8 μmol) of N,N-diisopropylethylamine and 10.4 mg (27.4 μmol) of HATU were added. The reaction mixture was stirred at RT for 10 min. 13.4 mg (27.4 μmol) of trifluoroacetic acid/N-(14-amino-3,6,9,12-tetraoxatetradec-1-yl)-2-(2,5-dioxo-2,5-dihydro-1H-pyrrol-1-ylacetamide (1:1) (Intermediate L85) dissolved in 4.0 μl (22.9 μmol) of N,N-diisopropylethylamine and 1.0 ml of DMF were then added. The reaction mixture was stirred at RT for 1 h. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 15.8 mg (68% of theory) of the compound 2-(trimethylsilyl)ethyl [23-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-2,19,22-trioxo-6,9,12,15-tetraoxa-3,18,23-triazahexacosan-26-yl]carbamate.

LC-MS (Method 1): Rₜ=1.35 min; MS (ESIpos): m/z=1011 (M+H)⁺.

15.1 mg (14.9 μmol) of 2-(trimethylsilyl)ethyl [23-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-2,19,22-trioxotrioxo-6,9,12,15-tetraoxa-3,18,23-triazahexacosan-26-yl]carbamate were dissolved in 2.0 ml of trifluoroethanol, and 12.2 mg (89.6 μmol) of zinc dichloride were added. The reaction mixture was stirred at 50° C. for 1 h. 26.2 mg (89.6 μmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 10.3 mg (70% of theory) of the title compound.

LC-MS (Method 1): Rₜ=0.88 min; MS (ESIpos): m/z=867 (M+H)⁺.

### Intermediate F273

### Trifluoroacetic acid/N-(3-aminopropyl)-N-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-2,19-dioxo-6,9,12,15-tetraoxa-22-thia-3,18-diazatetracosane-24-amide (1:1)

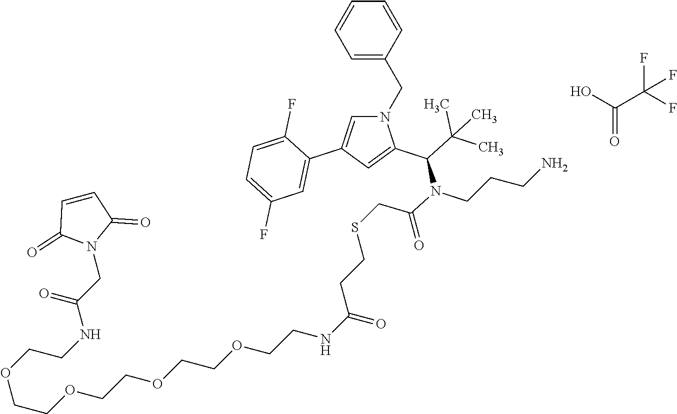 [SMILES: CC(C)(C)[C@H](c1cc(-c2cc(F)ccc2F)cn1Cc1ccccc1)N(CCCN)C(=O)CSCCC(=O)NCCOCCOCCOCCOCCNC(=O)CN1C(=O)C=CC1=O ; O=C(O)C(F)(F)F]

Under argon, 20.0 mg (28.5 μmol) of 11-{(1R)-1-[1-benzyl-4-(2,5-difluorophenyl)-1H-pyrrol-2-yl]-2,2-dimethylpropyl}-2,2-dimethyl-6,12-dioxo-5-oxa-14-thia-7,11-diaza-2-silaheptadecan-17-oic acid (Intermediate C69) were initially charged in 1.0 ml of DMF, and 10.0 μl (57.0 μmol) of N,N-diisopropylethylamine and 13.0 mg (34.2 μmol) of HATU were added. The reaction mixture was stirred at RT for 10 min. 16.7 mg (34.2 μmol) of trifluoroacetic acid/N-(14-amino-3,6,9,12-tetraoxatetradec-1-yl)-2-(2,5-dioxo-2,5-dihydro-1H-pyrrol-1-ylacetamide (1:1) (Intermediate L85) dissolved in 5.0 μl (28.5 μmol) of N,N-diisopropylethylamine and 1.0 ml of DMF were then added. The reaction mixture was stirred at RT for 1 h. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 18.6 mg (62% of theory) of the compound 2-(trimethylsilyl)ethyl [25-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-2,19,24-trioxo-6,9,12,15-tetraoxa-22-thia-3,18,25-triazaoctacosan-28-yl]-carbamate.

LC-MS (Method 1): Rₜ=1.37 min; MS (ESIpos): m/z=1057 (M+H)⁺.

17.1 mg (16.2 μmol) of 2-(trimethylsilyl)ethyl [25-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-2,19,24-trioxotrioxo-6,9,12,15-tetraoxa-22-thia-3,18,25-triazaoctacosan-28-yl]carbamate were dissolved in 2.0 ml of trifluoroethanol, and 13.2 mg (97.0 μmol) of zinc dichloride were added. The reaction mixture was stirred at 50° C. for 1 h. 28.4 mg (97.0 μmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 9.80 mg (59% of theory) of the title compound.

LC-MS (Method 1): Rₜ=0.88 min; MS (ESIpos): m/z=913 (M+H)⁺.

### Intermediate F274

### N-[(2,5-Dioxo-2,5-dihydro-1H-pyrrol-1-yl)acetyl]-L-valyl-L-alanyl-S-{2-[(3-aminopropyl){(1R)-1-[1-benzyl-4-(2,5-difluorophenyl)-1H-pyrrol-2-yl]-2,2-dimethylpropyl}amino]-2-oxoethyl}-L-cysteine/trifluoroacetic acid (1:1)

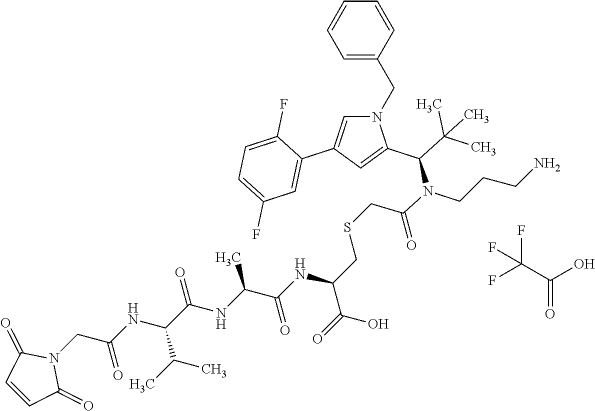

13.9 mg (0.0167 mmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine/trifluoroacetic acid (1:1) (Intermediate C71) were initially charged together with 7.07 mg (0.0217 mmol) of N-[(2,5-dioxo-2,5-dihydro-1H-pyrrol-1-yl)acetyl]-L-valyl-L-alanine (Intermediate L86) in 2.0 ml of acetonitrile. 23 μl (0.13 mmol) of N,N-diisopropylethylamine were then added, and 13 μl (0.022 mmol) of T3P (50% in ethyl acetate) were added dropwise. The reaction mixture was stirred at RT overnight. The reaction mixture was purified directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 3.70 mg (19% of theory) of the compound N-[(2,5-dioxo-2,5-dihydro-1H-pyrrol-1-yl)acetyl]-L-valyl-L-alanyl-S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine.

LC-MS (Method 1): Rₜ=1.34 min; MS (ESIpos): m/z=1024 (M+H)⁺.

10.6 mg (10.3 μmol) of N-[(2,5-dioxo-2,5-dihydro-1H-pyrrol-1-yl)acetyl]-L-valyl-L-alanyl-S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine were dissolved in 2.0 ml of trifluoroethanol, and 8.46 mg (62.1 μmol) of zinc dichloride were added. The reaction mixture was stirred at 50° C. for 1 h. 18.1 mg (62.1 μmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 5.60 mg (54% of theory) of the title compound.

LC-MS (Method 12): Rₜ=1.69 min; MS (ESIpos): m/z=880 (M+H)⁺.

### Intermediate F275

### N-[3-({2-[(3-Aminopropyl) {(1R)-1-[1-benzyl-4-(2,5-difluorophenyl)-1H-pyrrol-2-yl]-2,2-dimethylpropyl}amino]-2-oxoethyl}sulphanyl)propanoyl]-N-(2-{[(2,5-dioxo-2,5-dihydro-1H-pyrrol-1-yl)acetyl]amino}ethyl)-L-alpha-glutamine/trifluoroacetic acid (1:1)

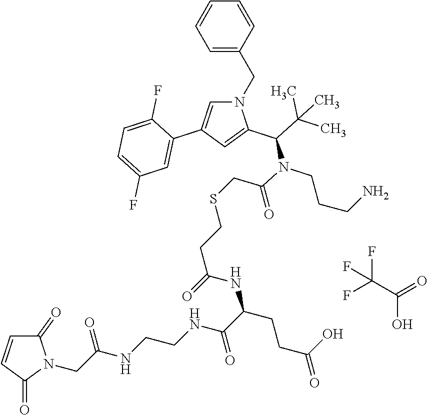 [SMILES: CC(C)(C)[C@H](c1cc(-c2cc(F)ccc2F)cn1Cc1ccccc1)N(CCCN)C(=O)CSCCC(=O)N[C@@H](CCC(=O)O)C(=O)NCCNC(=O)CN1C(=O)C=CC1=O ; O=C(O)C(F)(F)F]

39.0 mg (55.6 μmol) of 11-{(1R)-1-[1-benzyl-4-(2,5-difluorophenyl)-1H-pyrrol-2-yl]-2,2-dimethylpropyl}-2,2-dimethyl-6,12-dioxo-5-oxa-14-thia-7,11-diaza-2-silaheptadecan-17-oic acid (Intermediate C69) were initially charged in 4.0 ml of DMF, 41.6 mg (111 μmol) of 1-benzyl-5-[2-(trimethylsilyl)ethyl]-L-glutamate hydrochloride (1:1) (Intermediate L89), 29 μl (170 μmol) of N,N-diisopropylethylamine and 42.3 mg (111 μmol) of HATU were added and the mixture was stirred at RT for 1 hour. The reaction mixture was stirred at RT for 1 hour, quenched with acetic acid and purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 53.1 mg (93% of theory) of the compound 1-benzyl-5-[2-(trimethylsilyl)ethyl]-N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-L-glutamate.

LC-MS (Method 1): Rₜ=1.71 min; MS (ESIpos): m/z=1021 [M+H]⁺

Under argon, 7.60 mg (33.9 μmol) of palladium(II) acetate were initially charged in 3.0 ml of dichloromethane, and 14 μl (100 μmol) of triethylamine and 110 μl (680 μmol) of triethylsilane were added. The reaction mixture was stirred at RT for 5 min, and 69.2 mg (67.7 μmol) of 1-benzyl-5-[2-(trimethylsilyl)ethyl]-N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-L-glutamate dissolved in 3.0 ml of dichloromethane were added. The reaction mixture was stirred at RT overnight. The reaction mixture was filtered through a cardboard filter and the filter cake was washed with dichloromethane. The solvent was evaporated under reduced pressure. The residue was purified by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 38.4 mg (61% of theory) of the compound (19S)-11-{(1R)-1-[1-benzyl-4-(2,5-difluorophenyl)-1H-pyrrol-2-yl]-2,2-dimethylpropyl}-2,2-dimethyl-6,12,17-trioxo-19-{3-oxo-3-[2-(trimethylsilyl)ethoxy]propyl}-5-oxa-14-thia-7,11,18-triaza-2-silaicosan-20-oic acid.

LC-MS (Method 1): Rₜ=1.53 min; MS (ESIpos): m/z=931 (M+H)⁺.

10.0 mg (10.7 μmol) of (19S)-11-{(1R)-1-[1-benzyl-4-(2,5-difluorophenyl)-1H-pyrrol-2-yl]-2,2-dimethylpropyl}-2,2-dimethyl-6,12,17-trioxo-19-{3-oxo-3-[2-(trimethylsilyl)ethoxy]propyl}-5-oxa-14-thia-7,11,18-triaza-2-silaicosan-20-oic acid (Intermediate C69) were initially charged in 1.0 ml of DMF, 6.73 mg (21.5 μmol) of N-(2-aminoethyl)-2-(2,5-dioxo-2,5-dihydro-1H-pyrrol-1-yl)acetamide/2,2,2-trifluoroethane-1,1-diol (1:1) (Intermediate L1), 5.6 μl (32 μmol) of N,N-diisopropylethylamine and 8.17 mg (21.5 μmol) of HATU were added and the mixture was stirred at RT for 1 hour. The reaction mixture was stirred at RT for 3 hour, quenched with acetic acid and purified directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 6.90 mg (58% of theory) of the compound 2-(trimethylsilyl)ethyl N2-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-N-(2-{[(2,5-dioxo-2,5-dihydro-1H-pyrrol-1-yl)acetyl]amino}ethyl)-L-alpha-glutaminate.

LC-MS (Method 1): Rₜ=1.57 min; MS (ESIpos): m/z=1110 [M+H]⁺

6.90 mg (6.21 μmol) of 2-(trimethylsilyl)ethyl N²-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-N-(2-{[(2,5-dioxo-2,5-dihydro-1H-pyrrol-1-ylacetyl]amino}ethyl)-L-alpha-glutaminate were dissolved in 2.0 ml of trifluoroethanol, and 5.1 mg (37.2 μmol) zinc dichloride were added. The reaction mixture was stirred at 50° C. for 3 h. 5.1 mg (37.2 μmol) of zinc dichloride were added and the reaction mixture was stirred at 50° C. for 3 h. 5.1 mg (37.2 μmol) of zinc dichloride were added and the reaction mixture was stirred at 50° C. for 3 h. 10.1 mg (74.4 μmol) of zinc dichloride were added and the reaction mixture was stirred at 50° C. overnight and at RT for 72 h. 54.5 mg (186 μmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 2.4 mg (39% of theory) of the title compound.

LC-MS (Method 1): Rₜ=0.86 min; MS (ESIpos): m/z=866 (M+H)⁺.

### Intermediate F276

### S-{2-[(3-Aminopropyl){(1R)-1-[1-benzyl-4-(2,5-difluorophenyl)-1H-pyrrol-2-yl]-2,2-dimethylpropyl}amino]-2-oxoethyl}-N-{3-[2-(2-{[(2,5-dioxo-2,5-dihydro-1H-pyrrol-1-ylacetyl]amino}ethoxy)ethoxy]propanoyl}-L-cysteine/trifluoroacetic acid (1:1)

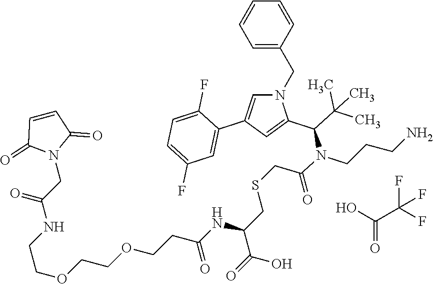

Under argon, 9.08 mg (28.9 μmol) of 3-[2-(2-{[(2,5-Dioxo-2,5-dihydro-1H-pyrrol-1-yl)acetyl]amino}ethoxy)ethoxy]propanoic acid (Intermediate L87) were initially charged in 1.0 ml of DMF, and 8.33 μl (48.2 μmol) of N,N-diisopropylethylamine and 11.0 mg (28.9 μmol) of HATU were added. The reaction mixture was stirred at RT for 10 min. 20.0 mg (27.7 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine/trifluoroacetic acid (1:1) (Intermediate C71) dissolved in 4.67 μl (24.1 μmol) of N,N-diisopropylethylamine and 1.0 ml of DMF were then added. The reaction mixture was stirred at RT for 1 h. The reaction mixture was purified directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 4.70 mg (19% of theory) of the compound S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-{3-[2-(2-{[(2,5-dioxo-2,5-dihydro-1H-pyrrol-1-yl)acetyl]amino}ethoxy)ethoxy]propanoyl}-L-cysteine.

LC-MS (Method 12): Rₜ=2.47 min; MS (ESIpos): m/z=1013 (M+H)⁺.

13.9 mg (13.7 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-{3-[2-(2-{[(2,5-dioxo-2,5-dihydro-1H-pyrrol-1-yl)acetyl]amino}ethoxy)ethoxy]propanoyl}-L-cysteine were dissolved in 2.0 ml of trifluoroethanol, and 5.6 mg (41.2 μmol) of zinc dichloride were added. The reaction mixture was stirred at 50° C. for 1 h. 5.6 mg (41.2 μmol) of zinc dichloride were added and the reaction mixture was stirred at 50° C. for 30 minutes. 24.1 mg (82.4 μmol) of ethylenediamine-N,N,N′,N′-tetraacetic acid were added and the reaction mixture was stirred for 10 min, and water (0.1% TFA) was then added. Purification was carried out directly by preparative RP-HPLC (column: Reprosil 250×30; 10μ, flow rate: 50 ml/min, MeCN/water, 0.1% TFA). The solvents were evaporated under reduced pressure and the residue was dried under high vacuum. This gave 10.8 mg (80% of theory) of the title compound.

LC-MS (Method 12): Rₜ=1.58 min; MS (ESIpos): m/z=869 (M+H)⁺.

### Intermediate F277

### N-[3-({2-[(3-Aminopropyl) {(1R)-1-[1-benzyl-4-(2,5-difluorophenyl)-1H-pyrrol-2-yl]-2,2-dimethylpropyl}amino]-2-oxoethyl}sulphanyl)propanoyl]-3-[(bromoacetyl)amino]-D-alanine/trifluoroacetic acid (1:1)

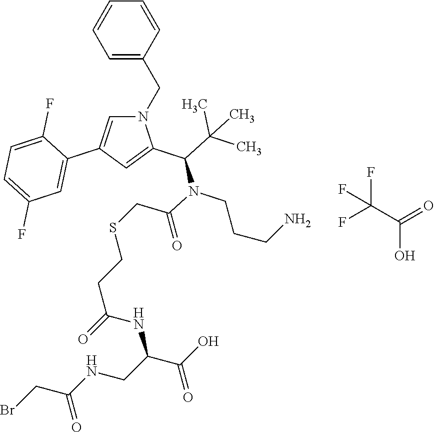 [SMILES: CC(C)(C)[C@H](c1cc(-c2cc(F)ccc2F)cn1Cc1ccccc1)N(CCCN)C(=O)CSCCC(=O)N[C@H](CNC(=O)CBr)C(=O)O ; O=C(O)C(F)(F)F]

8.90 mg (8.88 μmol) of trifluoroacetic acid/2-(trimethylsilyl)ethyl 3-amino-N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-D-alaninate (1:1) (Intermediate C80) and 2.31 mg (9.77 μmol) of 1-(2-bromoacetoxy)pyrrolidine-2,5-dione were dissolved in 1 ml of dimethylformamide, and 2.9 μl (27 μmol) of N-methylmorpholine were added. The reaction mixture was stirred at RT for 1 h. The reaction mixture was purified directly by preparative RP-HPLC (column: Reprosil 125×30; 10μ, flow rate: 50 ml/min, MeCN/water/0.1% TFA). The solvents were evaporated under reduced pressure and the residue was dried under high vacuum. This gave 5.80 mg (65% of theory) of the compound 2-(trimethylsilyl)ethyl N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-3-[(bromoacetyl)amino]-D-alaninate.

LC-MS (Method 1): Rₜ=1.57 min; MS (ESIpos): m/z=1008 (M+H)⁺.

5.80 mg (5.75 μmol) of 2-(trimethylsilyl)ethyl N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-3-[(bromoacetyl)amino]-D-alaninate were dissolved in 2.0 ml of trifluoroethanol, and 4.70 mg (34.5 μmol) of zinc dichloride were added. The reaction mixture was stirred at 50° C. for 3 h. 4.70 mg (34.5 μmol) of zinc dichloride were added and the reaction mixture was stirred at 50° C. for 5 h. 20.2 mg (69.0 μmol) of ethylenediamine-N,N,N′,N′-tetraacetic acid were added and the reaction mixture was stirred for 10 min, and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 1.70 mg (34% of theory) of the title compound.

LC-MS (Method 1): Rₜ=0.90 min; MS (ESIpos): m/z=764 (M+H)⁺.

### Intermediate F278

### N-[3-({2-[(3-Aminopropyl) {(1R)-1-[1-benzyl-4-(2,5-difluorophenyl)-1H-pyrrol-2-yl]-2,2-dimethylpropyl}amino]-2-oxoethyl}sulphanyl)propanoyl]-3-{[(2,5-dioxo-2,5-dihydro-1H-pyrrol-1-yl)acetyl]amino}-D-alanine/trifluoroacetic acid (1:1)

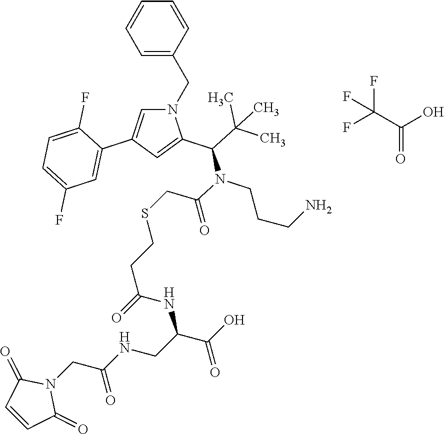 [SMILES: CC(C)(C)[C@H](c1cc(-c2cc(F)ccc2F)cn1Cc1ccccc1)N(CCCN)C(=O)CSCCC(=O)N[C@H](CNC(=O)CN1C(=O)C=CC1=O)C(=O)O ; O=C(O)C(F)(F)F]

10.0 mg (9.98 (0.1 mol) of trifluoroacetic acid/2-(trimethylsilyl)ethyl 3-amino-N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-D-alaninate (1:1) (Intermediate C80) and 2.77 mg (11.0 μmol) of 1-{2-[(2,5-dioxopyrrolidin-1-yl)oxy]-2-oxoethyl}-1H-pyrrole-2,5-dione were dissolved in 1 ml of dimethylformamide, and 3.3 μl (30 μmol) of N-methylmorpholine were added. The reaction mixture was stirred at RT overnight. 2.0 μl (35 μmol) of acetic acid were added, and the reaction mixture was purified directly by preparative RP-HPLC (column: Reprosil 125×30; 10μ, flow rate: 50 ml/min, MeCN/water/0.1% TFA). The solvents were evaporated under reduced pressure and the residue was dried under high vacuum. This gave 5.50 mg (54% of theory) of the compound 2-(trimethylsilyl)ethyl N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-3-{[(2,5-dioxo-2,5-dihydro-1H-pyrrol-1-yl)acetyl]amino}-D-alaninate.

LC-MS (Method 1): Rₜ=1.51 min; MS (ESIpos): m/z=1024 (M+H)⁺.

5.50 mg (5.36 μmol) of 2-(trimethylsilyl)ethyl N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-3-{[(2,5-dioxo-2,5-dihydro-1H-pyrrol-1-yl)acetyl]amino}-D-alaninate were dissolved in 1.0 ml of trifluoroethanol, and 4.39 mg (32.2 μmol) of zinc dichloride were added. The reaction mixture was stirred at 50° C. for 1 h. 4.39 mg (32.2 μmol) of zinc dichloride were added and the reaction mixture was stirred at 50° C. for 1 h. 4.39 mg (32.2 μmol) of zinc dichloride were added and the reaction mixture was stirred at 50° C. for 4 h. 28.2 mg (96.5 μmol) of ethylenediamine-N,N,N′,N′-tetraacetic acid were added and the reaction mixture was stirred for 10 min, and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 2.70 mg (56% of theory) of the title compound.

LC-MS (Method 1): Rₜ=0.89 min; MS (ESIpos): m/z=781 (M+H)⁺.

### Intermediate F279

### N-[6-(2,5-Dioxo-2,5-dihydro-1H-pyrrol-1-yl)hexanoyl]-L-valyl-N-[3-({(1R)-1-[1-benzyl-4-(2,5-difluorophenyl)-1H-pyrrol-2-yl]-2,2-dimethylpropyl}[({(2R)-2-carboxy-2-[(3-carboxypropanoyl)amino]ethyl}sulphanyl)acetyl]amino)propyl]-L- alaninamide

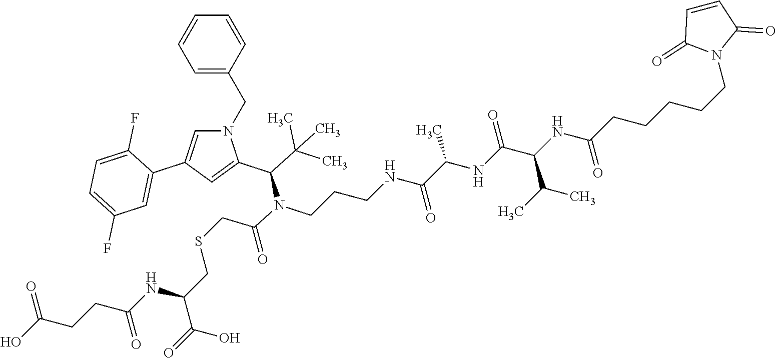 [SMILES: CC(C)[C@H](NC(=O)CCCCCN1C(=O)C=CC1=O)C(=O)N[C@@H](C)C(=O)NCCCN(C(=O)CSC[C@H](NC(=O)CCC(=O)O)C(=O)O)[C@@H](c1cc(-c2cc(F)ccc2F)cn1Cc1ccccc1)C(C)(C)C]

12.2 mg (14 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-(4-tert-butoxy-4-oxobutanoyl)-L-cysteine (Intermediate C77) were dissolved in 2.0 ml of trifluoroethanol, and 11.4 mg (83.8 μmol) of zinc dichloride were added. The reaction mixture was stirred at 50° C. for 3 h. 24.5 mg (83.8 μmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 4.60 mg (42% of theory) of the compound 4-{[(1R)-2-({2-[(3-aminopropyl){(1R)-1-[1-benzyl-4-(2,5-difluorophenyl)-1H-pyrrol-2-yl]-2,2-dimethylpropyl}amino]-2-oxoethyl}sulphanyl)-1-carboxyethyl]amino}-4-oxobutanoic acid/trifluoroacetic acid (1:1).

LC-MS (Method 1): Rₜ=0.88 min; MS (ESIpos): m/z=673 (M+H)⁺.

10.0 mg (12.7 μmol) of 4-{[(1R)-2-({2-[(3-aminopropyl){(1R)-1-[1-benzyl-4-(2,5-difluorophenyl)-1H-pyrrol-2-yl]-2,2-dimethylpropyl}amino]-2-oxoethyl}sulphanyl)-1-carboxyethyl]amino}-4-oxobutanoic acid/trifluoroacetic acid (1:1) and 7.41 mg (12.7 μmol) of 2,5-dioxopyrrolidin-1-yl N-[6-(2,5-dioxo-2,5-dihydro-1H-pyrrol-1-yl)hexanoyl]-L-valyl-L- alaninate (Intermediate L88) were dissolved in 1.5 ml of dimethylformamide, and 4.4 μl (25 μmol) of N,N-diisopropylethylamine were added. The reaction mixture was stirred at RT for 2 h. 2.0 μl (35 μmol) of acetic acid were added, and the reaction mixture was purified directly by preparative RP-HPLC (column: Reprosil 250×30; 10μ, flow rate: 50 ml/min, MeCN/water/0.1% TFA). The solvents were evaporated under reduced pressure and the residue was dried under high vacuum. This gave 5.20 mg (39% of theory) of the title compound.

LC-MS (Method 1): Rₜ=1.11 min; MS (ESIpos): m/z=1036 (M+H)⁺.

### Intermediate F280

### Trifluoroacetic acid/N-[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]-3-(2,5-dioxo-2,5-dihydro-1H-pyrrol-1-ylbenzamide (1:1)

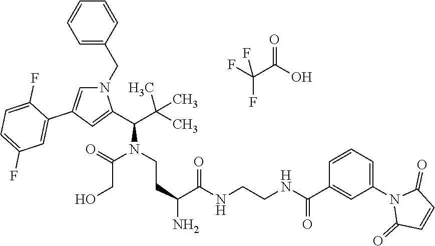 [SMILES: CC(C)(C)[C@H](c1cc(-c2cc(F)ccc2F)cn1Cc1ccccc1)N(CC[C@H](N)C(=O)NCCNC(=O)c1cccc(N2C(=O)C=CC2=O)c1)C(=O)CO ; O=C(O)C(F)(F)F]

The title compound was prepared from Intermediate C64 by coupling with commercially available 1-(3-{[(2,5-dioxopyrrolidin-1-yl)oxy]carbonyl}phenyl)-1H-pyrrole-2,5-dione and subsequent deprotection with zinc chloride.

LC-MS (Method 1): Rₜ=0.88 min; MS (ESIpos): m/z=755 (M+H)⁺.

### Intermediate F281

### N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-3-{[N-(bromoacetyl)-beta-alanyl]amino}-D-alanine/trifluoroacetic acid (1:1)

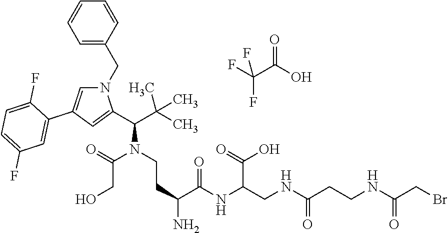 [SMILES: CC(C)(C)[C@H](c1cc(-c2cc(F)ccc2F)cn1Cc1ccccc1)N(CC[C@H](N)C(=O)NC(CNC(=O)CCNC(=O)CBr)C(=O)O)C(=O)CO ; O=C(O)C(F)(F)F]

First, the modified amino acid building blocks N-(bromoacetyl)-beta-alanine and 2-(trimethylsilyl)ethyl-3-amino-N-(tert-butoxycarbonyl)-D-alaninate were prepared by classical methods of peptide chemistry. These were then coupled in the presence of HATU and morpholine. The tert-butoxycarbonyl protective group was then removed using 10% strength trifluoroacetic acid in dichloromethane, giving the intermediate 2-(trimethylsilyl)ethyl 3-{[N-(bromoacetyl)-beta-alanyl]amino}-D-alaninate.

Finally, the title compound was prepared by coupling this intermediate with intermediate C58 in the presence of HATU and 4-methylmorpholine, followed by deprotection with zinc chloride.

LC-MS (Method 1): Rₜ=0.87 min; MS (ESIpos): m/z=791 and 793 (M+H)⁺.

### Intermediate F282

### Trifluoroacetic acid/(2S)-2-amino-4-[{(1R)-1-[1-benzyl-4-(2,5-difluorophenyl)-1H-pyrrol-2-yl]-2,2-dimethylpropyl}(glycoloyl)amino]-N-(3-{[N-(bromoacetyl)glycyl]amino}propylbutanamide (1:1)

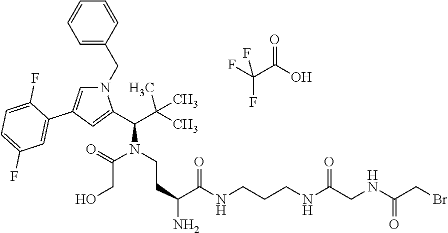 [SMILES: CC(C)(C)[C@H](c1cc(-c2cc(F)ccc2F)cn1Cc1ccccc1)N(CC[C@H](N)C(=O)NCCCNC(=O)CNC(=O)CBr)C(=O)CO ; O=C(O)C(F)(F)F]

First, the intermediate trifluoroacetic acid/N-(3-aminopropyl)-N2-(bromoacetyl)glycinamide (1:1) was prepared from tert-butyl glycinate and bromoacetic anhydride by classical methods of peptide chemistry.

Finally, the title compound was prepared by coupling this intermediate with intermediate C58 in the presence of HATU and 4-methylmorpholine, followed by deprotection with zinc chloride.

LC-MS (Method 1): Rₜ=0.83 min; MS (ESIpos): m/z=747 and 749 (M+H)⁺.

### Intermediate F283

### N-[(2R)-2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)-2-carboxyethyl]-N²-(bromoacetyl)-L-alpha-asparagine/trifluoroacetic acid (1:1)

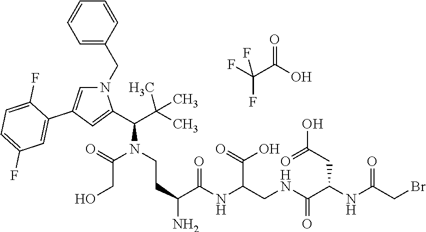 [SMILES: CC(C)(C)[C@H](c1cc(-c2cc(F)ccc2F)cn1Cc1ccccc1)N(CC[C@H](N)C(=O)NC(CNC(=O)[C@H](CC(=O)O)NC(=O)CBr)C(=O)O)C(=O)CO ; O=C(O)C(F)(F)F]

First, the modified amino acid building block (2S)-2-[(bromoacetyl)amino]-4-oxo-4-[2-(trimethylsilyl)ethoxy]butanoic acid and bromoacetic anhydride was prepared from (2S)-2-amino-4-oxo-4-[2-(trimethylsilyl)ethoxy]butanoic acid and bromoacetic anhydride and the amino acid building block 2-(trimethylsilyl)ethyl-3-amino-N-(tert-butoxycarbonyl)-D-alaninate was prepared from commercially available 3-{[(benzyloxy)carbonyl]amino}-N-(tert-butoxycarbonyl)-D-alanine/N-cyclohexylcyclohexanamine (1:1). Both building blocks were coupled in the presence of HATU and morpholine and the tert-butoxycarbonyl protective group was then removed using 5% strength trifluoroacetic acid in dichloromethane, giving the silylethyl ester protective groups and thus the intermediate trifluoroacetic acid/2-(trimethylsilyl)ethyl-N-{(2R)-2-amino-3-oxo-3-[2-(trimethylsilyl)ethoxy]propyl}-N2-(bromoacetyl)-L-alpha-asparaginate (1:1). Finally, the title compound was prepared by coupling this intermediate with intermediate C58 in the presence of HATU and 4-methylmorpholine, followed by deprotection with zinc chloride.

LC-MS (Method 1): Rₜ=0.84 min; MS (ESIpos): m/z=835 and 837 (M+H)⁺.

### Intermediate F284

### N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-3-{[1-(2,5-dioxo-2,5-dihydro-1H-pyrrol-1-yl)-2,18-dioxo-6,9,12,15-tetraoxa-3-azaoctadecan-18-yl]amino}-D-alanine/trifluoroacetic acid (1:1)

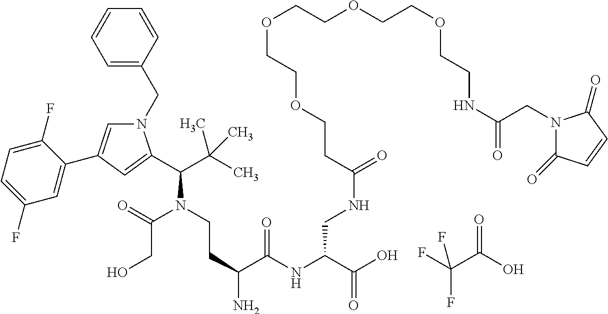

First, intermediate L80 was coupled with commercially available (2,5-dioxo-2,5-dihydro-1H-pyrrol-1-yl)acetic acid in the presence of HATU and N,N-diisopropylethylamine, and the tert-butoxycarbonyl protective group was then removed using 16% strength trifluoroacetic acid in dichloromethane, giving the silylethyl ester protective group.

Finally, the title compound was prepared by coupling this intermediate with intermediate C58 in the presence of HATU and N,N-diisopropylethylamine, followed by deprotection with zinc chloride.

LC-MS (Method 12): Rₜ=1.46 min; MS (ESIpos): m/z=984.45 (M+H)⁺.

### Intermediate F285

### N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-3-[(18-bromo-17-oxo-4,7,10,13-tetraoxa-16-azaoctadecan-1-oyl)amino]-D-alanine/trifluoroacetic acid (1:1)

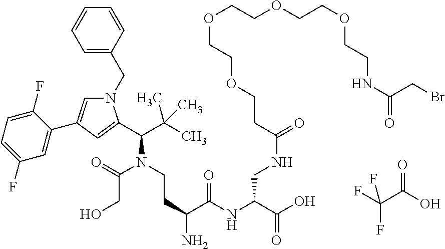 [SMILES: CC(C)(C)[C@H](c1cc(-c2cc(F)ccc2F)cn1Cc1ccccc1)N(CC[C@H](N)C(=O)N[C@H](CNC(=O)CCOCCOCCOCCOCCNC(=O)CBr)C(=O)O)C(=O)CO ; O=C(O)C(F)(F)F]

First, intermediate L80 was acylated with commercially available bromoacetic anhydride, and the tert-butoxycarbonyl protective group was then removed using 20% strength trifluoroacetic acid in dichloromethane, giving the silylethyl ester protective group.

Finally, the title compound was prepared by coupling this intermediate with intermediate C58 in the presence of HATU and N,N-diisopropylethylamine, followed by deprotection with zinc chloride.

LC-MS (Method 1): Rₜ=0.85 min; MS (ESIpos): m/z=967 and 969 (M+H)⁺.

### Intermediate F286

### 1-[(N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-3-{[(2,5-dioxo-2,5-dihydro-1H-pyrrol-1-yl) acetyl]amino}-D-alanyl)amino]-3,6,9,12-tetraoxapentadecan-15-oic acid/trifluoroacetic acid (1:1)

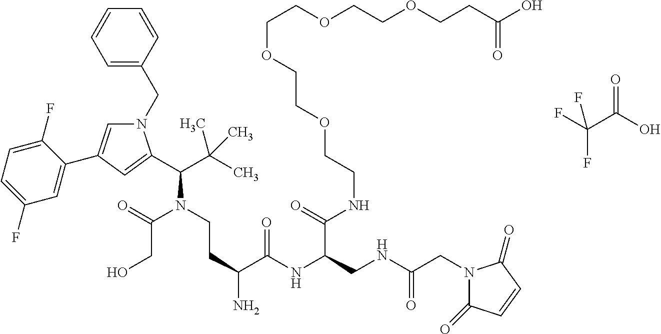 [SMILES: CC(C)(C)[C@H](c1cc(-c2cc(F)ccc2F)cn1Cc1ccccc1)N(CC[C@H](N)C(=O)N[C@H](CNC(=O)CN1C(=O)C=CC1=O)C(=O)NCCOCCOCCOCCOCCC(=O)O)C(=O)CO ; O=C(O)C(F)(F)F]

First, intermediate L91 was coupled with (2,5-dioxo-2,5-dihydro-1H-pyrrol-1-yl)acetic acid in the presence of HATU and N,N-diisopropylethylamine, and the Boc protective group was then removed using 12.5% strength TFA in DCM. The resulting intermediate was coupled with intermediate C58 in the presence of HATU and N,N-diisopropylethylamine and then converted into the title compound by deprotection with zinc chloride.

LC-MS (Method 1): Rₜ=0.84 min; MS (ESIpos): m/z=984 (M+H)⁺.

### Intermediate F288

### N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-3-({N-[(2,5-dioxo-2,5-dihydro-1H-pyrrol-1-yl) acetyl]-L- seryl}amino)-D-alanine/trifluoroacetic acid (1:1)

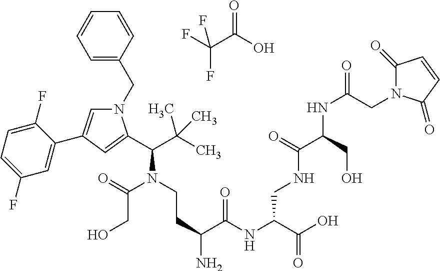 [SMILES: CC(C)(C)[C@H](c1cc(-c2cc(F)ccc2F)cn1Cc1ccccc1)N(CC[C@H](N)C(=O)N[C@H](CNC(=O)[C@H](CO)NC(=O)CN1C(=O)C=CC1=O)C(=O)O)C(=O)CO ; O=C(O)C(F)(F)F]

35 mg (39 (0.1 mol) of intermediate C74 were coupled in the presence of HATU and N,N-diisopropyethylamine with N-[(2,5-dioxo-2,5-dihydro-1H-pyrrol-1-yl)acetyl]-L-serine which had been prepared beforehand from tert-butyl O-tert-butyl-L-serinate and (2,5-dioxo-2,5-dihydro-1H-pyrrol-1-yl)acetic acid. Deprotection with zinc chloride and purification by HPLC gave 14 mg (38% of theory) of the title compound.

LC-MS (Method 12): Rₜ=1.43 min; MS (ESIpos): m/z=824.34 (M+H)⁺.

### Intermediate F289

### N²-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-N⁶—[(2,5-dioxo-2,5-dihydro-1H-pyrrol-1-yl)acetyl]-D-lysine/trifluoroacetate (1:1)

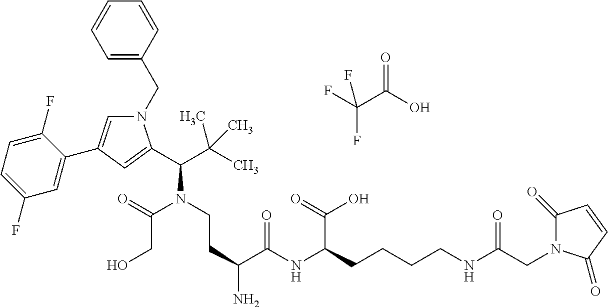 [SMILES: CC(C)(C)[C@H](c1cc(-c2cc(F)ccc2F)cn1Cc1ccccc1)N(CC[C@H](N)C(=O)N[C@H](CCCCNC(=O)CN1C(=O)C=CC1=O)C(=O)O)C(=O)CO ; O=C(O)C(F)(F)F]

First, trifluoroacetic acid/2-(trimethylsilyl)ethyl-N⁶-[(2,5-dioxo-2,5-dihydro-1H-pyrrol-1-yl)acetyl]-D-lysinate (1:1) was prepared by classical methods of peptide chemistry from N⁶-[(benzyloxy)carbonyl]-N²-(tert-butoxycarbonyl)-D-lysine.

12.5 mg (25 μmol) of this intermediate were then coupled in the presence of HATU and 4-methylmorpholine with 15 mg (23 μmol) of Intermediate C58. Deprotection with zinc chloride and purification by HPLC gave 14 mg (53% of theory) of the title compound.

LC-MS (Method 1): Rₜ=0.83 min; MS (ESIpos): m/z=779 (M+H)⁺.

### Intermediate F290

### N²-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-N⁶-(bromoacetyl)-D-lysine/trifluoroacetic acid (1:1)

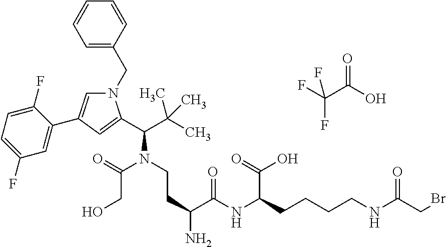 [SMILES: CC(C)(C)[C@H](c1cc(-c2cc(F)ccc2F)cn1Cc1ccccc1)N(CC[C@H](N)C(=O)N[C@H](CCCCNC(=O)CBr)C(=O)O)C(=O)CO ; O=C(O)C(F)(F)F]

First, trifluoroacetic acid/2-(trimethylsilyl)ethyl-N6-(bromoacetyl)-D-lysinate (1:1) was prepared by classical methods of peptide chemistry from N⁶-[(benzyloxy)carbonyl]-N²-(tert-butoxycarbonyl)-D-lysine.

12 mg (25 μmol) of this intermediate were then coupled in the presence of HATU and 4-methylmorpholine with 15 mg (23 μmol) of Intermediate C58. Deprotection with zinc chloride and purification by HPLC gave 7 mg (36% of theory) of the title compound.

LC-MS (Method 1): Rₜ=0.86 min; MS (ESIpos): m/z=762 and 764 (M+H)⁺.

### Intermediate F291

### N-[(2,5-Dioxo-2,5-dihydro-1H-pyrrol-1-yl)acetyl]-L-valyl-N-{3-[{(1R)-1-[1-benzyl-4-(2,5-difluorophenyl)-1H-pyrrol-2-yl]-2,2-dimethylpropyl}(glycoloyl)amino]propyl}-L-alaninamide

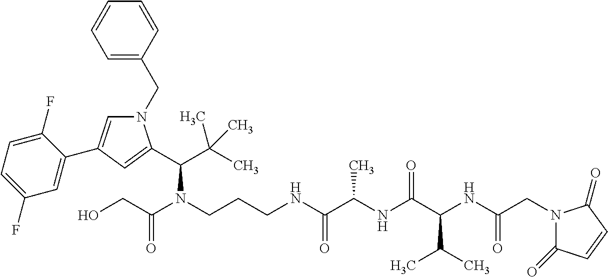 [SMILES: CC(C)[C@H](NC(=O)CN1C(=O)C=CC1=O)C(=O)N[C@@H](C)C(=O)NCCCN(C(=O)CO)[C@@H](c1cc(-c2cc(F)ccc2F)cn1Cc1ccccc1)C(C)(C)C]

The title compound was prepared from Example M9 first by coupling with N-[(benzyloxy)carbonyl]-L-valyl-L-alanine in the presence of HATU and N,N-diisopropylethylamine. In the next step, the Z protective group was removed by hydrogenating for 1 hour over 10% palladium on activated carbon at RT under hydrogen standard pressure and then converting the deprotected intermediate into the title compound by coupling with (2,5-dioxo-2,5-dihydro-1H-pyrrol-1-yl)acetic acid in the presence of HATU and N,N-diisopropylethylamine.

LC-MS (Method 1): Rₜ=1.21 min; MS (ESIpos): m/z=777 (M+H)⁺.

### Intermediate F293

### N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-3-{[(2,5-dioxo-2,5-dihydro-1H-pyrrol-1-yl)benzoyl]amino}-D-alanine/trifluoroacetic acid (1:1)

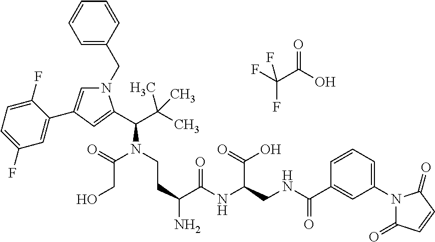 [SMILES: CC(C)(C)[C@H](c1cc(-c2cc(F)ccc2F)cn1Cc1ccccc1)N(CC[C@H](N)C(=O)N[C@H](CNC(=O)c1cccc(N2C(=O)C=CC2=O)c1)C(=O)O)C(=O)CO ; O=C(O)C(F)(F)F]

35 mg (39 μmol) of Intermediate C74 were dissolved in 4 ml of DMF and, in the presence of N,N-diisopropylethylamine, coupled with 13.5 mg (43 μmol) of commercially available 1-(3-{[(2,5-dioxopyrrolidin-1-yl)oxy]carbonyl}phenyl)-1H-pyrrole-2,5-dione. Deprotection with zinc chloride and purification by HPLC gave 12 mg (34% of theory) of the title compound.

LC-MS (Method 12): Rₜ=0.93 min; MS (ESIpos): m/z=799 (M+H)⁺.

### Intermediate F294

### N-{5-[(2,5-Dioxopyrrolidin-1-yl)oxy]-5-oxopentanoyl}-L-valyl-N-{(1S)-3-[{(1R)-1-[1-benzyl-4-(2,5-difluorophenyl)-1H-pyrrol-2-yl]-2,2-dimethylpropyl}(glycoloyl)amino]-1-carboxypropyl}-L-alaninamide

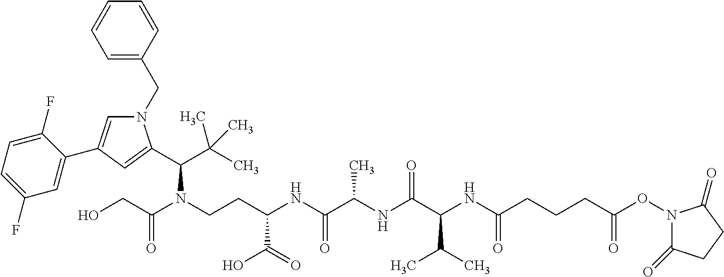 [SMILES: CC(C)[C@H](NC(=O)CCCC(=O)ON1C(=O)CCC1=O)C(=O)N[C@@H](C)C(=O)N[C@@H](CCN(C(=O)CO)[C@@H](c1cc(-c2cc(F)ccc2F)cn1Cc1ccccc1)C(C)(C)C)C(=O)O]

41 mg (0.05 mmol) of Intermediate C76 dissolved in 12 ml of methanol were hydrogenated over 10 mg of 10% palladium on activated carbon at RT for 1 h under hydrogen standard pressure. The catalyst was then filtered off and the solvent was removed under reduced pressure. This gave 32 mg (92% of theory) of the deprotected intermediate.

15 mg (0.022 mmol) of this intermediate were dissolved in DMF, and 13 mg (0.039 mmol) of 1,1′-[(1,5-dioxopentan-1,5-diyl)bis(oxy)]dipyrrolidine-2,5-dione and 7 μl of N,N-diisopropylethylamine were added. After 1 h of stirring at RT, the reaction was concentrated and the residue was purified by HPLC. This gave 9 mg (45% of theory) of the title compound.

LC-MS (Method 1): Rₜ=1.08 min; MS (ESIpos): m/z=895 (M+H)⁺.

### Intermediate F295

### N-[(2,5-Dioxo-2,5-dihydro-1H-pyrrol-1-yl)acetyl]-L-valyl-N-{(1S)-3-[{(1R)-1-[1-benzyl-4-(2,5-difluorophenyl)-1H-pyrrol-2-yl]-2,2-dimethylpropyl}(glycoloyl)amino]-1-carboxypropyl}-L-alaninamide

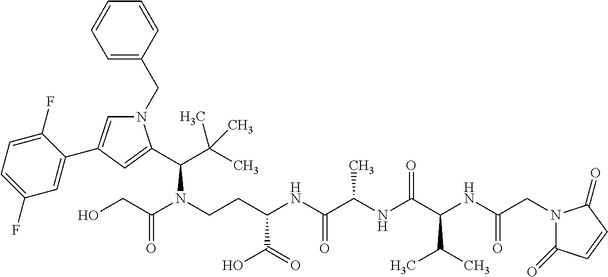 [SMILES: CC(C)[C@H](NC(=O)CN1C(=O)C=CC1=O)C(=O)N[C@@H](C)C(=O)N[C@@H](CCN(C(=O)CO)[C@@H](c1cc(-c2cc(F)ccc2F)cn1Cc1ccccc1)C(C)(C)C)C(=O)O]

41 mg (0.05 mmol) of Intermediate C76 dissolved in 12 ml of methanol were hydrogenated over 10 mg of 10% palladium on activated carbon at RT for 1 h under hydrogen standard pressure. The catalyst was then filtered off and the solvent was removed under reduced pressure. This gave 32 mg (92% of theory) of the deprotected intermediate.

15 mg (0.022 mmol) of this intermediate were dissolved in 4 ml of DMF, and 10 mg (0.039 mmol) of 1-{2-[(2,5-dioxopyrrolidin-1-yl)oxy]-2-oxoethyl}-1H-pyrrole-2,5-dione and 7 μl of N,N-diisopropylethylamine were added. After 2 h of stirring at RT, the reaction was concentrated and the residue was purified by HPLC. This gave 10 mg (56% of theory) of the title compound.

LC-MS (Method 1): Rₜ=1.08 min; MS (ESIpos): m/z=821 (M+H)⁺.

### Intermediate F296

### Trifluoroacetic acid/(2S)-2-amino-4-[{(1R)-1-[1-benzyl-4-(2,5-difluorophenyl)-1H-pyrrol-2-yl]-2,2-dimethylpropyl}(glycoloyl)amino]-N-{2-[(2-{[(2,5-dioxo-2,5-dihydro-1H-pyrrol-1-yl) acetyl]-amino}ethyl)sulphonyl]ethyl}butanamide (1:1)

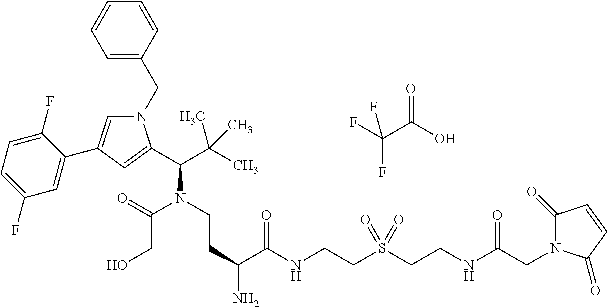 [SMILES: CC(C)(C)[C@H](c1cc(-c2cc(F)ccc2F)cn1Cc1ccccc1)N(CC[C@H](N)C(=O)NCCS(=O)(=O)CCNC(=O)CN1C(=O)C=CC1=O)C(=O)CO ; O=C(O)C(F)(F)F]

The title compound was prepared from Intermediate L81 by coupling with Intermediate C58 in the presence of HATU and N,N-diisopropylethylamine. In the next step, the Z protective group was removed by hydrogenation over 10% palladium on activated carbon in DCM/methanol 1:1 at RT under hydrogen standard pressure for 30 min. The deprotected intermediate was then converted by coupling with (2,5-dioxo-2,5-dihydro-1H-pyrrol-1-ylacetic acid in the presence of HATU and N,N-diisopropylethylamine and finally by deprotection with zinc chloride into the title compound.

LC-MS (Method 1): Rₜ=0.83 min; MS (ESIpos): m/z=785 (M+H)⁺.

### Intermediate F297

### S-{2-[{(1R)-1-[1-Benzyl-4-(2,5-difluorophenyl)-1H-pyrrol-2-yl]-2,2-dimethylpropyl}(pyrrolidin-3-ylmethyl)amino]-2-oxoethyl}-N-[6-(2,5-dioxo-2,5-dihydro-1H-pyrrol-1-yl)hexanoyl]-L-cysteine/trifluoroacetic acid (1:1) (Isomer 1)

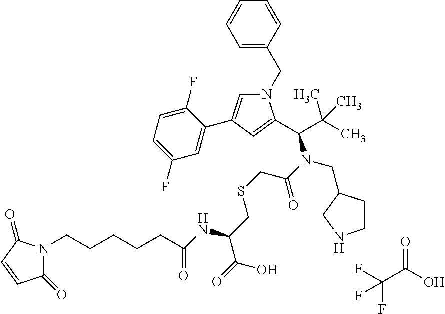

Under argon, 15 mg (0.11 mmol) of zinc chloride were added to a solution of 36 mg (0.03 mmol, 68% pure) of S-[2-({(1R)-1-[1-benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]-N-[6-(2,5-dioxo-2,5-dihydro-1H-pyrrol-1-yl)hexanoyl]-L-cysteine (Intermediate C92) in 0.74 ml of 2,2,2-trifluoroethanol, and the reaction mixture was stirred at 50° C. for 7 h. 32 mg (0.11 mmol) of EDTA were then added and the mixture was stirred for 15 minutes. Ethyl acetate was added to the reaction mixture and the organic phase was washed repeatedly with water and with saturated NaCl solution. The organic phase was dried over magnesium sulphate and the solvent was evaporated under reduced pressure. The residue was purified by preparative HPLC. This gave 6.4 mg (25% of theory) of the title compound.

LC-MS (Method 1): Rₜ=0.95 min; MS (ESIpos): m/z=792 (M+H—CF₃CO₂H)⁺.

### Intermediate F298

### S-{2-[{(1R)-1-[1-Benzyl-4-(2,5-difluorophenyl)-1H-pyrrol-2-yl]-2,2-dimethylpropyl}(pyrrolidin-3-ylmethyl)amino]-2-oxoethyl}-N-[6-(2,5-dioxo-2,5-dihydro-1H-pyrrol-1-yl)hexanoyl]-L-cysteine/trifluoroacetic acid (1:1) (isomer 2)

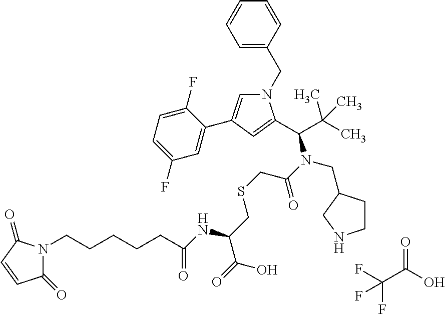

Under argon, 19 mg (0.14 mmol) of zinc chloride were added to a solution of 45 mg (0.04 mmol, 71% pure) of S-[2-({(1R)-1-[1-benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]-N-[6-(2,5-dioxo-2,5-dihydro-1H-pyrrol-1-yl)hexanoyl]-L-cysteine (intermediate C91) in 0.94 ml of 2,2,2-trifluoroethanol, and the reaction mixture was stirred at 50° C. for 3 h. 42 mg (0.14 mmol) of EDTA were then added, and the mixture was stirred for 15 minutes. Ethyl acetate was added to the reaction mixture and the organic phase was washed repeatedly with water and sat. NaCl solution. The organic phase was dried over magnesium sulphate and the solvent was evaporated under reduced pressure. The residue was purified by preparative HPLC. This gave 5.7 mg (18% of theory) of the title compound.

LC-MS (Method 1): Rₜ=0.96 min; MS (ESIpos): m/z=791 (M+H—CF₃CO₂H)⁺.

### Intermediate F299

### S-(2-{(3-Aminopropyl) [(R)-[1-benzyl-4-(2,5-difluorophenyl)-1H-pyrrol-2-yl](cyclohexyl) methyl]amino}-2-oxoethyl)-N-[6-(2,5-dioxo-2,5-dihydro-1H-pyrrol-1-yl)hexanoyl]-L-cysteine/trifluoroacetic acid (1:1)

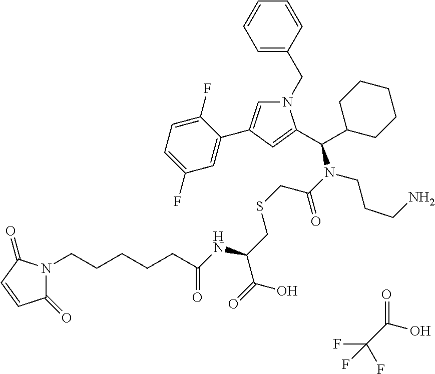 [SMILES: NCCCN(C(=O)CSC[C@H](NC(=O)CCCCCN1C(=O)C=CC1=O)C(=O)O)[C@@H](c1cc(-c2cc(F)ccc2F)cn1Cc1ccccc1)C1CCCCC1 ; O=C(O)C(F)(F)F]

76.8 mg (0.57 mmol) of zinc chloride were added to a solution of 88.0 mg (0.09 mmol) of S-{11-[(R)-[1-benzyl-4-(2,5-difluorophenyl)-1H-pyrrol-2-yl](cyclohexyl)methyl]-2,2-dimethyl-6,12-dioxo-5-oxa-7,11-diaza-2-silatridecan-13-yl}-N-[6-(2,5-dioxo-2,5-dihydro-1H-pyrrol-1-yl)hexanoyl]-L-cysteine (intermediate C84) in 1.88 ml of 2,2,2-trifluoroethanol, and the reaction mixture was stirred at 50° C. for 3 h. 164.6 mg (0.57 mmol) of EDTA were then added, and the mixture was stirred for 15 minutes. Ethyl acetate was added to the reaction mixture and the org. phase was washed repeatedly with water and with sat. NaCl solution. The organic phase was dried over sodium sulphate and the solvent was evaporated under reduced pressure. The residue was purified by preparative HPLC. This gave 31 mg (35% of theory) of the title compound.

LC-MS (Method 12): Rₜ=1.82 min; MS (ESIpos): m/z=792 (M+H)⁺.

### Intermediate F300

### (2S)-2-Amino-4-[{(1R)-1-[1-benzyl-4-(2,5-difluorophenyl)-1H-pyrrol-2-yl]-2,2-dimethylpropyl}(glycoloyl)amino]-N-(2-{[(2R)-2-(2,5-dioxo-2,5-dihydro-1H-pyrrol-1-yl)propanoyl]amino}ethylbutanamide

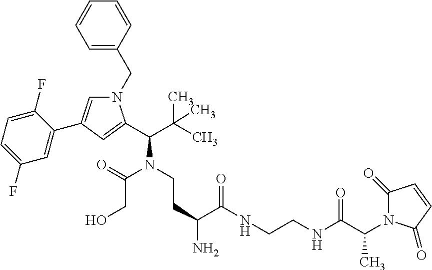 [SMILES: C[C@H](C(=O)NCCNC(=O)[C@@H](N)CCN(C(=O)CO)[C@@H](c1cc(-c2cc(F)ccc2F)cn1Cc1ccccc1)C(C)(C)C)N1C(=O)C=CC1=O]

Under argon, 11 mg (0.08 mmol) of zinc chloride were added to a solution of 7 mg (0.08 mmol) of 2-(trimethylsilyl)ethyl-{(2S)-4-[{(1R)-1-[1-benzyl-4-(2,5-difluorophenyl)-1H-pyrrol-2-yl]-2,2-dimethylpropyl}(glycoloyl)amino]-1-[(2-{[(2R)-2-(2,5-dioxo-2,5-dihydro-1H-pyrrol-1-yl)propanoyl]amino}ethylamino]-1-oxobutan-2-yl}carbamate (intermediate C100) in 0.2 ml of 2,2,2-trifluoroethanol, and the reaction mixture was stirred at 50° C. for 8 h. 14 mg (0.05 mmol) of EDTA were then added, and the mixture was stirred for 15 minutes. Ethyl acetate was added to the reaction mixture and the organic phase was washed repeatedly with water and with sat. NaCl solution. The organic phase was dried over magnesium sulphate and the solvent was evaporated under reduced pressure. The residue was purified by preparative HPLC. This gave 1.6 mg (27% of theory) of the title compound.

LC-MS (Method 1): Rₜ=0.88 min; MS (ESIpos): m/z=707 (M+H—CF₃CO₂H)⁺.

### Intermediate F302

### S-{2-[{(1R)-1-[1-Benzyl-4-(2,5-difluorophenyl)-1H-pyrrol-2-yl]-2,2-dimethylpropyl}(pyrrolidin-3-ylmethyl)amino]-2-oxoethyl}-N-[(2,5-dioxo-2,5-dihydro-1H-pyrrol-1-yl)acetyl]-L-cysteine/trifluoroacetate (1:1) (isomer 1)

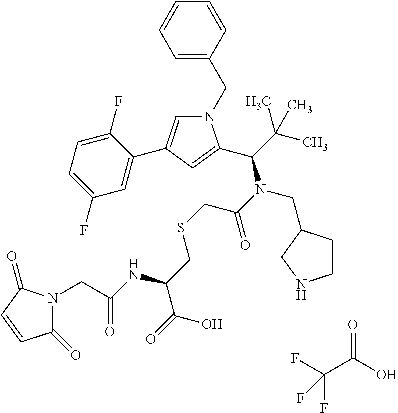 [SMILES: CC(C)(C)[C@H](c1cc(-c2cc(F)ccc2F)cn1Cc1ccccc1)N(CC1CCNC1)C(=O)CSC[C@H](NC(=O)CN1C(=O)C=CC1=O)C(=O)O ; O=C(O)C(F)(F)F]

Under argon, 31.7 mg (0.23 mmol) of zinc chloride were added to a mixture of 56.9 mg (58.2 mmol, 85% pure) of S-[2-({(1R)-1-[1-benzyl-4-(2,5-difluorophenyl)-1H-pyrrol-2-yl]-2,2-dimethylpropyl}{[(1-(tert-butoxycarbonyl)pyrrolidin-3-yl]methyl}amino)-2-oxoethyl]-N-[(2,5-dioxo-2,5-dihydro-1H-pyrrol-1-yl)acetyl]-L-cysteine (intermediate C94) in 1.4 ml of 2,2,2-trifluoroethanol, and the reaction mixture was stirred at 50° C. for 3 h. 68.0 mg (0.23 mmol) of EDTA were then added, and the mixture was stirred for 15 minutes. Ethyl acetate was added to the reaction mixture and the organic phase was washed repeatedly with water and with sat. NaCl solution. The organic phase was dried over magnesium sulphate and the solvent was evaporated under reduced pressure. The residue was purified by preparative HPLC. This gave 7 mg (13% of theory) of the title compound.

LC-MS (Method 1): Rₜ=0.91 min; MS (ESIpos): m/z=736 (M+H—CF₃CO₂H)⁺.

### Intermediate F304

### N-(2-{[3-({2-[{(1R)-1-[1-Benzyl-4-(2,5-difluorophenyl)-1H-pyrrol-2-yl]-2,2-dimethylpropyl}(pyrrolidin-3-ylmethyl)amino]-2-oxoethyl}sulphanyl)propanoyl]amino}ethyl)-6-(2,5-dioxo-2,5-dihydro-1H-pyrrol-1-ylhexanamide (1:1) trifluoroacetic acid (isomer 2)

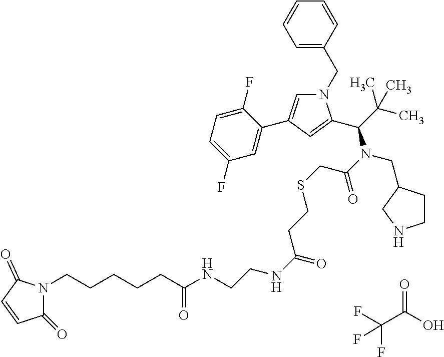 [SMILES: CC(C)(C)[C@H](c1cc(-c2cc(F)ccc2F)cn1Cc1ccccc1)N(CC1CCNC1)C(=O)CSCCC(=O)NCCNC(=O)CCCCCN1C(=O)C=CC1=O ; O=C(O)C(F)(F)F]

13.2 mg (0.10 mmol) of zinc chloride were added to a solution of 22.3 mg (0.02 mmol) of tert-butyl 3-[2-{(1R)-1-[1-benzyl-4-(2,5-difluorophenyl)-1H-pyrrol-2-yl]-2,2-dimethylpropyl}-18-(2,5-dioxo-2,5-dihydro-1H-pyrrol-1-yl)-3,8,13-trioxo-5-thia-2,9,12-triazaoctadec-1-yl]pyrrolidine-1-carboxylate (intermediate 98) in 0.64 ml of 2,2,2-trifluoroethanol, and the reaction mixture was stirred at 50° C. for 8 h. 28.36 mg (0.10 mmol) of EDTA were then added, and the mixture was stirred for 15 minutes. Ethyl acetate was added to the reaction mixture and the organic phase was washed repeatedly with water and with sat. NaCl solution. The organic phase was dried over magnesium sulphate and the solvent was evaporated under reduced pressure. The residue was purified by prep. HPLC. This gave 5 mg (24% of theory) of the title compound.

LC-MS (Method 5): Rₜ 3.05 min; MS (ESIpos): m/z=819 (M+H—CF₃CO₂H)⁺.

### Intermediate F305

### N-{(1R)-1-[1-Benzyl-4-(2,5-difluorophenyl)-1H-pyrrol-2-yl]-2,2-dimethylpropyl}-22-(2,5-dioxo-2,5-dihydro-1H-pyrrol-1-yl)-6,17-dioxo-N-(pyrrolidin-3-ylmethyl)-10,13-dioxa-3-thia-7,16-diazadocosane-1-amide (1:1) trifluoroacetic acid (isomer 2)

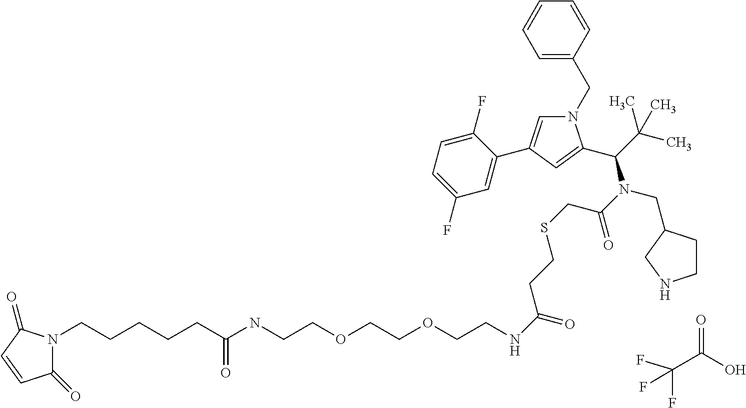

13.42 mg (0.10 mmol) of zinc chloride were added to a solution of 24.80 mg (0.02 mmol) of tert-butyl 3-[2-{(1R)-1-[1-benzyl-4-(2,5-difluorophenyl)-1H-pyrrol-2-yl]-2,2-dimethylpropyl}-24-(2,5-dioxo-2,5-dihydro-1H-pyrrol-1-yl)-3,8,19-trioxo-12,15-dioxa-5-thia-2,9,18-triazatetracos-1-yl]pyrrolidine-1-carboxylate (intermediate C99) in 0.65 ml of 2,2,2-trifluoroethanol, and the reaction mixture was stirred at 50° C. for 8 h. 28.78 mg (0.10 mmol) of EDTA were then added, and the mixture was stirred for 15 minutes. Ethyl acetate was added to the reaction mixture and the org. phase was washed repeatedly with water and with sat. NaCl solution. The organic phase was dried over magnesium sulphate and the solvent was evaporated under reduced pressure. The residue was purified by preparative HPLC. This gave 10 mg (44% of theory) of the title compound.

LC-MS (Method 5): Rₜ=3.11 min; MS (ESIpos): m/z=907 (M+H—CF₃CO₂H)⁺.

### Intermediate F306

### N⁶—(N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-beta-alanyl)-N²—{N-[(2,5-dioxo-2,5-dihydro-1H-pyrrol-1-yl)acetyl]-L-valyl-L-alanyl-beta-alanyl}-L-lysine/trifluoroacetic acid (1:1)

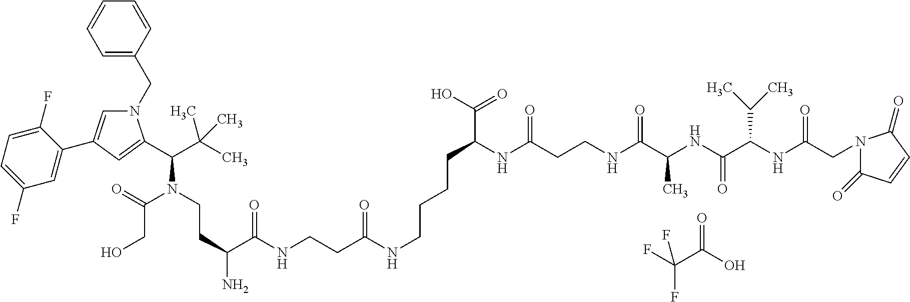

The title compound was prepared by coupling 24 mg (0.029 mmol) of the intermediate C61 with 30 mg (0.035 mmol) of intermediate L99 in the presence of 16.7 mg (0.044 mmol) of HATU and 15 μl of N,N-diisopropylethylamine and subsequent deprotection with zinc chloride in trifluoroethanol as described for intermediate F119. Purification by preparative HPLC gave 19 mg (52% of theory over 2 steps) of the title compound.

LC-MS (Method 1): Rₜ=0.84 min; MS (ESIpos): m/z=1091 (M+H)⁺.

### Intermediate F307

### N-[6-(2,5-Dioxo-2,5-dihydro-1H-pyrrol-1-yl)hexanoyl]-L-valyl-L-alanyl-S-{(5R,14R)-13-(3-aminopropyl)-14-[1-benzyl-4-(2,5-difluorophenyl)-1H-pyrrol-2-yl]-5-carboxy-15,15-dimethyl-2,7,12-trioxo-10-thia-3,6,13-triazahexadec-1-yl}-L-cysteine/trifluoroacetic acid (1:1)

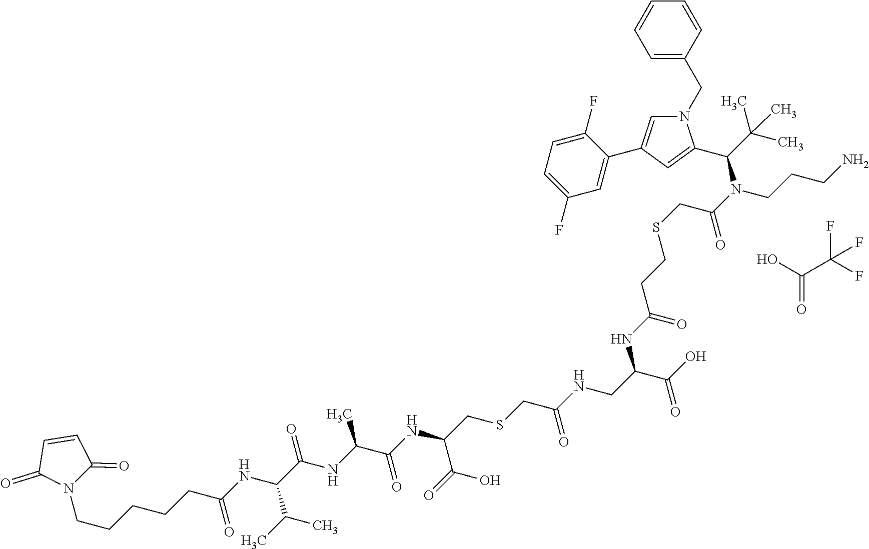

8.90 mg (8.88 μmol) of trifluoroacetic acid/2-(trimethylsilyl)ethyl 3-amino-N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-D-alaninate (1:1) (intermediate C80) and 2.31 mg (9.77 μmol) of 1-(2-bromoacetoxy)pyrrolidine-2,5-dione were dissolved in 1 ml of dimethylformamide, and 2.9 μl (27 μmol) of N-methylmorpholine were added. The reaction mixture was stirred at RT for 1 h. The reaction mixture was purified directly by prep. RP-HPLC (column: Reprosil 125×30; 10μ, flow rate: 50 ml/min, MeCN/water/0.1% TFA). The solvents were evaporated under reduced pressure and the residue was dried under high vacuum. This gave 5.80 mg (65% of theory) of the compound 2-(trimethylsilyl)ethyl N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-3-[(bromoacetyl)amino]-D-alaninate.

LC-MS (Method 1): Rₜ=1.57 min; MS (ESIpos): m/z=1008 (M+H)⁺.

2-(Trimethylsilyl)ethyl N-(11-{(1R)-1-[1-benzyl-4-(2,5-difluorophenyl)-1H-pyrrol-2-yl]-2,2-dimethylpropyl}-2,2-dimethyl-6,12,17-trioxo-5-oxa-14-thia-7,11-diaza-2-silaheptadecan-17-yl)-3-[(bromoacetyl)amino]-D-alaninate (31.9 mg, 31.6 μmol) and L-cysteine (7.66 mg, 63.2 μmol) were dissolved in 3.0 ml of DMF and stirred at RT overnight. The reaction mixture was purified directly by prep. RP-HPLC (column: Reprosil 250×30; 10μ, flow rate: 50 ml/min, MeCN/water/0.1% TFA). The solvents were evaporated under reduced pressure and the residue was dried under high vacuum. This gave 28.1 mg (76% of theory) of the compound S-[(19R)-11-{(1R)-1-[1-benzyl-4-(2,5-difluorophenyl)-1H-pyrrol-2-yl]-2,2-dimethylpropyl}-2,2-dimethyl-6,12,17,22-tetraoxo-19-{[2-(trimethylsilyl)ethoxy]carbonyl}-5-oxa-14-thia-7,11,18,21-tetraaza-2-silatricosan-23-yl]-L-cysteine/trifluoroacetic acid (1:1).

LC-MS (Method 12): Rₜ=2.52 min; MS (ESIpos): m/z=1049 [M+H]⁺

S-[(19R)-11-{(1R)-1-[1-Benzyl-4-(2,5-difluorophenyl)-1H-pyrrol-2-yl]-2,2-dimethylpropyl}-2,2-dimethyl-6,12,17,22-tetraoxo-19-{[2-(trimethylsilyl)ethoxy]carbonyl}-5-oxa-14-thia-7,11,18,21-tetraaza-2-silatricosan-23-yl]-L-cysteine/trifluoroacetic acid (1:1) (13.5 mg, 11.6 μmol) was dissolved in 1.0 ml of DMF, 2,5-dioxopyrrolidin-1-yl N-[6-(2,5-dioxo-2,5-dihydro-1H-pyrrol-1-yl)hexanoyl]-L-valyl-L-alaninate (6.76 mg, 11.6 μmol) (intermediate L88) and N,N-diisopropylethylamine (4.0 μl, 23 μmol) were added and the mixture was stirred at RT for 1 h. The reaction mixture was purified directly by prep. RP-HPLC (column: Reprosil 250×30; 10μ, flow rate: 50 ml/min, MeCN/water/0.1% TFA). The solvents were evaporated under reduced pressure and the residue was dried under high vacuum. This gave 11.1 mg (68% of theory) of the compound N-[6-(2,5-dioxo-2,5-dihydro-1H-pyrrol-1-yl)hexanoyl]-L-valyl-L-alanyl-S-[(19R)-11-{(1R)-1-[1-benzyl-4-(2,5-difluorophenyl)-1H-pyrrol-2-yl]-2,2-dimethylpropyl}-2,2-dimethyl-6,12,17,22-tetraoxo-19-{[2-(trimethylsilyl)ethoxy]carbonyl}-5-oxa-14-thia-7,11,18,21-tetraaza-2-silatricosan-23-yl]-L-cysteine.

LC-MS (Method 14): Rₜ=7.38 min; MS (ESIpos): m/z=1412 [M+H]⁺

N-[6-(2,5-Dioxo-2,5-dihydro-1H-pyrrol-1-yl)hexanoyl]-L-valyl-L-alanyl-S-[(19R)-11-{(1R)-1-[1-benzyl-4-(2,5-difluorophenyl)-1H-pyrrol-2-yl]-2,2-dimethylpropyl}-2,2-dimethyl-6,12,17,22-tetraoxo-19-{[2-(trimethylsilyl)ethoxy]carbonyl}-5-oxa-14-thia-7,11,18,21-tetraaza-2-silatricosan-23-yl]-L-cysteine (9.40 mg, 6.65 μmol) was dissolved in 2.0 ml of trifluoroethanol, and zinc dichloride (5.44 mg, 39.9 μmol) was added. The reaction mixture was stirred at 50° C. for 1 h. The zinc dichloride (5.44 mg, 39.9 μmol) was added, and the reaction mixture was stirred at 50° C. for 1 h. Ethylenediamine-N,N,N′,N′-tetraacetic acid (23.4 mg, 79.8 μmol) was added, the reaction mixture was stirred for 10 min and water (0.1% TFA) was then added. Purification was carried out directly by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 5.60 mg (66% of theory) of the title compound.

LC-MS (Method 1): Rₜ=0.93 min; MS (ESIpos): m/z=1168 (M+H)⁺.

### Intermediate F308

### N-[6-(2,5-Dioxo-2,5-dihydro-1H-pyrrol-1-yl)hexanoyl]-L-valyl-N-[(12R,19R)-19-amino-4-{(1R)-1-[1-benzyl-4-(2,5-difluorophenyl)-1H-pyrrol-2-yl]-2,2-dimethylpropyl}-12,19-dicarboxy-5,10,15-trioxo-7,17-dithia-4,11,14-triazanonadec-1-yl]-L-alaninamide/trifluoroacetic acid (1:1)

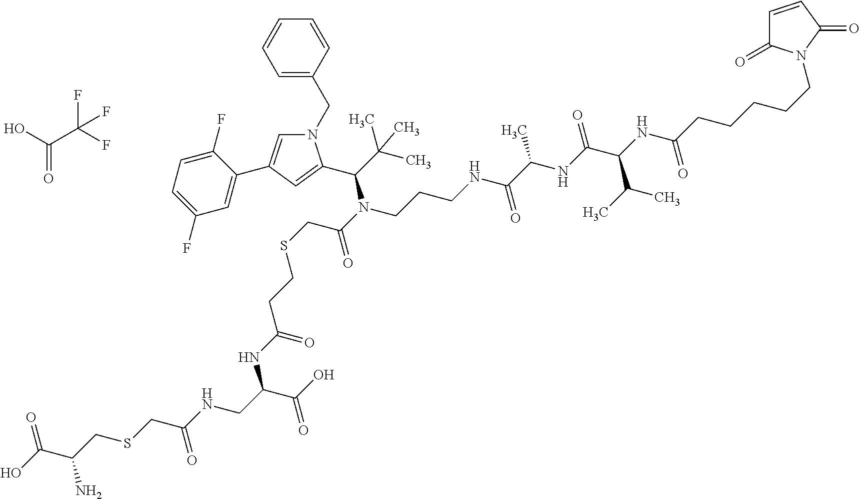 [SMILES: CC(C)[C@H](NC(=O)CCCCCN1C(=O)C=CC1=O)C(=O)N[C@@H](C)C(=O)NCCCN(C(=O)CSCCC(=O)N[C@H](CNC(=O)CSC[C@H](N)C(=O)O)C(=O)O)[C@@H](c1cc(-c2cc(F)ccc2F)cn1Cc1ccccc1)C(C)(C)C ; O=C(O)C(F)(F)F]

N-[3-({2-[(3-Aminopropyl) {(1R)-1-[1-benzyl-4-(2,5-difluorophenyl)-1H-pyrrol-2-yl]-2,2-dimethylpropyl}amino]-2-oxoethyl}sulphanyl)propanoyl]-3-[(bromoacetyl)amino]-D-alanine/trifluoroacetic acid (1:1) (12.7 mg, 14.5 μmol) and N-{[2-(trimethylsilyl)ethoxy]carbonyl}-L-cysteine (3.84 mg, 14.5 μmol) were dissolved in 1.5 ml of DMF, and the mixture was stirred at RT overnight.

N,N-Diisopropylethylamine (2.5 μl, 14 μmol) was then added. The reaction mixture was stirred at RT for 3 h, and water (0.1% TFA) was then added. Purification was carried out directly by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 7.40 mg (48% of theory) of the compound S-{(5R,14R)-13-(3-aminopropyl)-14-[1-benzyl-4-(2,5-difluorophenyl)-1H-pyrrol-2-yl]-5-carboxy-15,15-dimethyl-2,7,12-trioxo-10-thia-3,6,13-triazahexadec-1-yl}-N-{[2-(trimethylsilyl)ethoxy]carbonyl}-L- cysteine/trifluoro acetic acid (1:1).

LC-MS (Method 1): Rₜ=1.03 min; MS (ESIpos): m/z=949 [M+H]⁺

S-{(5R,14R)-13-(3-Aminopropyl)-14-[1-benzyl-4-(2,5-difluorophenyl)-1H-pyrrol-2-yl]-5-carboxy-15,15-dimethyl-2,7,12-trioxo-10-thia-3,6,13-triazahexadec-1-yl}-N-{[2-(trimethylsilyl)ethoxy]carbonyl}-L-cysteine/trifluoroacetic acid (1:1) (7.50 mg, 7.05 μmol) was dissolved in 1.0 ml of DMF, and 2,5-dioxopyrrolidin-1-yl N-[6-(2,5-dioxo-2,5-dihydro-1H-pyrrol-1-yl)hexanoyl]-L-valyl-L-alaninate (4.11 mg, 82% pure, 7.05 μmol) (intermediate L88) and N,N-diisopropylethylamine (2.5 μl, 14 μmol) were added. The reaction mixture was stirred at RT for 1 h and then purified directly by prep. RP-HPLC (column: Reprosil 250×30; 10μ, flow rate: 50 ml/min, MeCN/water/0.1% TFA). The solvents were evaporated under reduced pressure and the residue was dried under high vacuum. This gave 4.30 mg (46%) of the compound N-[6-(2,5-dioxo-2,5-dihydro-1H-pyrrol-1-yl)hexanoyl]-L-valyl-N-[(8R,15R)-23-{(1R)-1-[1-benzyl-4-(2,5-difluorophenyl)-1H-pyrrol-2-yl]-2,2-dimethylpropyl}-8,15-dicarboxy-2,2-dimethyl-6,12,17,22-tetraoxo-5-oxa-10,20-dithia-7,13,16,23-tetraaza-2-silahexacosan-26-yl]-L-alaninamide.

LC-MS (Method 14): Rₜ=6.47 min; MS (ESIpos): m/z=1312 [M+H]⁺

N-[6-(2,5-Dioxo-2,5-dihydro-1H-pyrrol-1-yl)hexanoyl]-L-valyl-N-[(8R,15R)-23-{(1R)-1-[1-benzyl-4-(2,5-difluorophenyl)-1H-pyrrol-2-yl]-2,2-dimethylpropyl}-8,15-dicarboxy-2,2-dimethyl-6,12,17,22-tetraoxo-5-oxa-10,20-dithia-7,13,16,23-tetraaza-2-silahexacosan-26-yl]-L-alaninamide (4.00 mg, 3.05 μmol) was dissolved in 1.0 ml of trifluoroethanol, and zinc dichloride (2.49 mg, 18.3 μmol) was added. The reaction mixture was stirred at 50° C. for 1 h, ethylenediamine-N,N,N′,N′-tetraacetic acid (5.34 mg, 18.3 μmol) was then added, the mixture was stirred for 10 min and water (0.1% TFA) was then added. Purification was carried out directly by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 2.50 mg (64% of theory) of the title compound.

LC-MS (Method 1): Rₜ=1.00 min; MS (ESIpos): m/z=1168 [M+H]⁺

### Intermediate F309

### 4-{[(11R,17R)-16-(3-Aminopropyl)-17-[1-benzyl-4-(2,5-difluorophenyl)-1H-pyrrol-2-yl]-1-(2,5-dioxo-2,5-dihydro-1H-pyrrol-1-yl)-18,18-dimethyl-6,6-dioxido-2,10,15-trioxo-6lambda⁶,13-dithia-3,9,16-triazanonadecan-11-yl]amino}-4-oxobutanoic acid/trifluoro acetic acid (1:1)

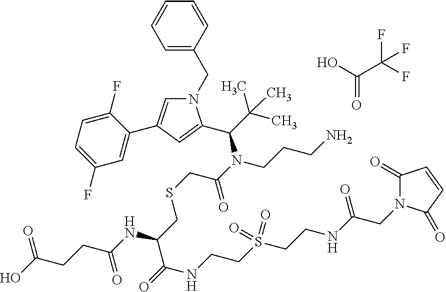 [SMILES: CC(C)(C)[C@H](c1cc(-c2cc(F)ccc2F)cn1Cc1ccccc1)N(CCCN)C(=O)CSC[C@H](NC(=O)CCC(=O)O)C(=O)NCCS(=O)(=O)CCNC(=O)CN1C(=O)C=CC1=O ; O=C(O)C(F)(F)F]

S-(11-{(1R)-1-[1-Benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-(4-tert-butoxy-4-oxobutanoyl)-L-cysteine (50.0 mg, 57.3 μmol) (intermediate C77) and trifluoroacetic acid/benzyl {2-[(2-aminoethyl)sulphonyl]ethyl}carbamate (1:1) (27.5 mg, 68.7 μmol) (intermediate L81) were initially charged in 4.0 ml of DMF, and HATU (26.1 mg, 68.7 μmol) and N,N-diisopropylethylamine: (30 μl, 170 μmol) were added. The reaction mixture was stirred at RT for 10 min and then purified directly by prep. RP-HPLC (column: Reprosil 250×30; 10μ, flow rate: 50 ml/min, MeCN/water/0.1% TFA). The solvents were evaporated under reduced pressure and the residue was dried under high vacuum. This gave 53.9 mg (81%) of the compound tert-butyl 4-{[(12R)-17-{(1R)-1-[1-benzyl-4-(2,5-difluorophenyl)-1H-pyrrol-2-yl]-2,2-dimethylpropyl}-26,26-dimethyl-7,7-dioxido-3,11,16,22-tetraoxo-1-phenyl-2,23-dioxa-7lambda6,14-dithia-4,10,17,21-tetraaza-26-silaheptacosan-12-yl]amino}-4-oxobutanoate.

LC-MS (Method 1): Rₜ=1.54 min; MS (ESIpos): m/z=1141 [M+H]⁺

Under argon, palladium(II) acetate (5.12 mg, 22.8 μmol) was initially charged in 3.0 ml of DCM, triethylamine (9.5 μl, 68 μmol) and triethylsilane (73 μl, 460 μmol) were added and the mixture was stirred for 5 min. tert-Butyl 4-{[(12R)-17-{(1R)-1-[1-benzyl-4-(2,5-difluorophenyl)-1H-pyrrol-2-yl]-2,2-dimethylpropyl}-26,26-dimethyl-7,7-dioxido-3,11,16,22-tetraoxo-1-phenyl-2,23-dioxa-7lambda⁶,14-dithia-4,10,17,21-tetraaza-26-silaheptacosan-12-yl]amino}-4-oxobutanoate (52.1 mg, 45.6 μmol) in 2.0 ml of DCM was then added. The reaction mixture was stirred at RT overnight, and 2.0 ml of water were added. The solvents were evaporated under reduced pressure. Acetonitrile was added to the residue, the mixture was filtered and the product was purified by prep. RP-HPLC (column: Reprosil 125×30; 10μ, flow rate: 50 ml/min, MeCN/water/0.1% TFA). The solvents were evaporated under reduced pressure and the residue was dried under high vacuum. This gave 43.4 mg (85%) of the compound trifluoroacetic acid/tert-butyl 4-{[(16R)-23-amino-11-{(1R)-1-[1-benzyl-4-(2,5-difluorophenyl)-1H-pyrrol-2-yl]-2,2-dimethylpropyl}-2,2-dimethyl-21,21-dioxido-6,12,17-trioxo-5-oxa-14,21lambda6-dithia-7,11,18-triaza-2-silatricosan-16-yl]amino}-4-oxobutanoate (1:1).

LC-MS (Method 1): Rₜ=1.21 min; MS (ESIpos): m/z=1007 [M+H]⁺

Trifluoroacetic acid/tert-butyl 4-{[(16R)-23-amino-11-{(1R)-1-[1-benzyl-4-(2,5-difluorophenyl)-1H-pyrrol-2-yl]-2,2-dimethylpropyl}-2,2-dimethyl-21,21-dioxido-6,12,17-trioxo-5-oxa-14,21lambda⁶-dithia-7,11,18-triaza-2-silatricosan-16-yl]amino}-4-oxobutanoate (1:1) (20.0 mg, 17.8 μmol) and (2,5-dioxo-2,5-dihydro-1H-pyrrol-1-ylacetic acid (3.32 mg, 21.4 μmol) were initially charged in 2.0 ml of DMF, and HATU (8.14 mg, 21.4 μmol) and N,N-diisopropylethylamine (9.3 μl, 54 μmol) were added.

The reaction mixture was stirred at RT for 10 min. The reaction mixture was purified directly by prep. RP-HPLC (column: Reprosil 250×30; 10μ, flow rate: 50 ml/min, MeCN/water/0.1% TFA). The solvents were evaporated under reduced pressure and the residue was dried under high vacuum. This gave 17.4 mg (85%) of the compound tert-butyl 4-{[(16R)-11-{(1R)-1-[1-benzyl-4-(2,5-difluorophenyl)-1H-pyrrol-2-yl]-2,2-dimethylpropyl}-26-(2,5-dioxo-2,5-dihydro-1H-pyrrol-1-yl)-2,2-dimethyl-21,21-dioxido-6,12,17,25-tetraoxo-5-oxa-14,21lambda6-dithia-7,11,18,24-tetraaza-2-silahexacosan-16-yl]amino}-4-oxobutanoate.

LC-MS (Method 1): Rₜ=1.46 min; MS (ESIpos): m/z=1144 [M+H]⁺

tert-Butyl 4-{[(16R)-11-{(1R)-1-[1-benzyl-4-(2,5-difluorophenyl)-1H-pyrrol-2-yl]-2,2-dimethylpropyl}-26-(2,5-dioxo-2,5-dihydro-1H-pyrrol-1-yl)-2,2-dimethyl-21,21-dioxido-6,12,17,25-tetraoxo-5-oxa-14,21lambda⁶-dithia-7,11,18,24-tetraaza-2-silahexacosan-16-yl]amino}-4-oxobutanoate (15.9 mg, 13.9 μmol) was dissolved in 2.0 ml of trifluoroethanol, and zinc dichloride (11.4 mg, 83.4 μmol) was added. The reaction mixture was stirred at 50° C. for 1 h. Zinc dichloride (11.4 mg, 83.4 μmol) was added and the reaction mixture was stirred at 50° C. for 1 h. Zinc dichloride (11.4 mg, 83.4 μmol) was added and the reaction mixture was stirred at 50° C. for 1 h. Ethylenediamine-N,N,N′,N′-tetraacetic acid (73.2 mg, 250 μmol) was added, the reaction mixture was stirred for 10 min and water (0.1% TFA) was then added. Purification was carried out directly by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 10 mg (68% of theory) of the title compound.

LC-MS (Method 12): Rₜ=1.45 min; MS (ESIpos): m/z=944 [M+H]⁺

### Intermediate F310

### Trifluoroacetic acid/N-[(8R,14R)-13-(3-aminopropyl)-14-[1-benzyl-4-(2,5-difluorophenyl)-1H-pyrrol-2-yl]-1-(2,5-dioxo-2,5-dihydro-1H-pyrrol-1-yl)-15,15-dimethyl-2,7,12-trioxo-10-thia-3,6,13-triazahexadecan-8-yl]-2,5,8,11-tetraoxatetradecane-14-amide (1:1)

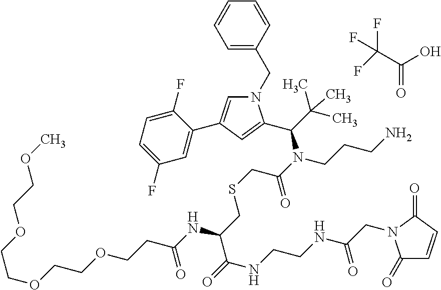

S-(11-{(1R)-1-[1-Benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine/trifluoroacetic acid (1:1) (100 mg, 120 μmol) (intermediate C70) and 1-[(14-oxo-2,5,8,11-tetraoxatetradecan-14-yl)oxy]pyrrolidine-2,5-dione (44.1 mg, 132 μmol) were initially charged in 3.0 ml of DMF, and 4-methylmorpholine (40 μl, 360 μmol) was added. The reaction mixture was stirred at RT overnight, quenched with acetic acid (420 μmol) and purified directly by prep. RP-HPLC (column: Reprosil 125×30; 10μ, flow rate: 50 ml/min, MeCN/water/0.1% TFA). The solvents were evaporated under reduced pressure and the residue was dried under high vacuum. This gave 69.4 mg (62% of theory) of the compound S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-(14-oxo-2,5,8,11-tetraoxatetradecan-14-yl)-L-cysteine.

LC-MS (Method 12): Rₜ=2.61 min; MS (ESIneg): m/z=933 [M−H]⁻

S-(11-{(1R)-1-[1-Benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-(14-oxo-2,5,8,11-tetraoxatetradecan-14-yl)-L-cysteine (27.0 mg, 28.9 μmol) was initially charged in 2.0 ml of DMF, and N-(2-aminoethyl)-2-(2,5-dioxo-2,5-dihydro-1H-pyrrol-1-yl)acetamide (11.4 mg, 57.7 μmol) (intermediate L1), N,N-diisopropylethylamine (15 μl, 87 μmol) and HATU (22.0 mg, 57.7 μmol) were added. The reaction mixture was stirred at RT for 3 h and purified directly by prep. RP-HPLC (column: Reprosil 250×30; 10μ, flow rate: 50 ml/min, MeCN/water/0.1% TFA). The solvents were evaporated under reduced pressure and the residue was dried under high vacuum. This gave 13.7 mg (43% of theory) of the compound 2-(trimethylsilyl)ethyl {(16R)-21-{(1R)-1-[1-benzyl-4-(2,5-difluorophenyl)-1H-pyrrol-2-yl]-2,2-dimethylpropyl}-16-[(2-{[(2,5-dioxo-2,5-dihydro-1H-pyrrol-1-yl)acetyl]amino}ethylcarbamoyl]-14,20-dioxo-2,5,8,11-tetraoxa-18-thia-15,21-diazatetracosan-24-yl}carbamate.

LC-MS (Method 12): Rₜ=2.54 min; MS (ESIpos): m/z=1114 [M+H]⁺

2-(Trimethylsilyl)ethyl {(16R)-21-{(1R)-1-[1-benzyl-4-(2,5-difluorophenyl)-1H-pyrrol-2-yl]-2,2-dimethylpropyl}-16-[(2-{[(2,5-dioxo-2,5-dihydro-1H-pyrrol-1-yl) acetyl]amino}ethylcarbamoyl]-14,20-dioxo-2,5,8,11-tetraoxa-18-thia-15,21-diazatetracosan-24-yl}carbamate (13.7 mg, 12.3 μmol) was dissolved in 2.0 ml of trifluoroethanol, and zinc dichloride (10.1 mg, 73.8 μmol) was added. The reaction mixture was stirred at 50° C. for 4 h. Ethylenediamine-N,N,N′,N′-tetraacetic acid (21.6 mg, 73.8 μmol) was added, the reaction mixture was stirred for 10 min and water (0.1% TFA) was then added. Purification was carried out directly by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 7.30 mg (47% of theory) of the title compound.

LC-MS (Method 1): Rₜ=1.01 min; MS (ESIpos): m/z=970 [M+H]⁺

### Intermediate F311

### S-{2-[(3-Aminopropyl){(1R)-1-[1-benzyl-4-(2,5-difluorophenyl)-1H-pyrrol-2-yl]-2,2-dimethylpropyl}amino]-2-oxoethyl}-N-[1-(2,5-dioxo-2,5-dihydro-1H-pyrrol-1-yl)-2,30-dioxo-6,9,12,15,18,21,24,27-octaoxa-3-azatriacontan-30-yl]-L-cysteine/trifluoroacetic acid (1:1)

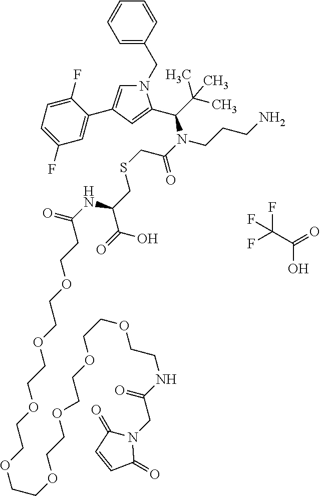 [SMILES: CC(C)(C)[C@H](c1cc(-c2cc(F)ccc2F)cn1Cc1ccccc1)N(CCCN)C(=O)CSC[C@H](NC(=O)CCOCCOCCOCCOCCOCCOCCOCCOCCNC(=O)CN1C(=O)C=CC1=O)C(=O)O ; O=C(O)C(F)(F)F]

1-(2,5-Dioxo-2,5-dihydro-1H-pyrrol-1-yl)-2-oxo-6,9,12,15,18,21,24,27-octaoxa-3-azatriacontan-30-oic acid (10.8 mg, 18.7 μmol) (intermediate L97) was initially charged in 1.0 ml of DMF, N,N-diisopropylethylamine (5.4 μl, 31.2 μmol) and HATU (7.10 mg, 18.7 μmol) were added and the mixture was stirred for 10 min. S-(11-{(1R)-1-[1-Benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine/trifluoroacetic acid (1:1) (12.9 mg, 15.6 μmol) (intermediate C71), dissolved in 1.0 ml of DMF and N,N-diisopropylethylamine (2.7 μl, 15.6 μmol), was then added. The reaction mixture was stirred at RT for 2 h and then purified directly by prep. RP-HPLC (column: Reprosil 125×30; 10μ, flow rate: 50 ml/min, MeCN/water/0.1% TFA). The solvents were evaporated under reduced pressure and the residue was dried under high vacuum. This gave 3.5 mg (18%) of the compound S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[1-(2,5-dioxo-2,5-dihydro-1H-pyrrol-1-yl)-2,30-dioxo-6,9,12,15,18,21,24,27-octaoxa-3-azatriacontan-30-yl]-L-cysteine.

LC-MS (Method 1): Rₜ=1.30 min; MS (ESIneg): m/z=1276 [M−H]⁻

S-(11-{(1R)-1-[1-Benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[1-(2,5-dioxo-2,5-dihydro-1H-pyrrol-1-yl)-2,30-dioxo-6,9,12,15,18,21,24,27-octaoxa-3-azatriacontan-30-yl]-L-cysteine (3.50 mg, 2.74 μmol) was dissolved in 1.0 ml of trifluoroethanol, and zinc dichloride (6.25 mg, 16.4 μmol) was added. The reaction mixture was stirred at 50° C. for 4 h. Ethylenediamine-N,N,N′,N′-tetraacetic acid (47 μl, 16 μmol) was added, the reaction mixture was stirred for 10 min and water (0.1% TFA) was then added. Purification was carried out directly by prep. RP-HPLC (column: Reprosil 125×30; 10μ, flow rate: 50 ml/min, MeCN/water, 0.1% TFA). The solvents were evaporated under reduced pressure and the residue was dried under high vacuum. This gave 2.0 mg (59% of theory) of the title compound.

LC-MS (Method 1): Rₜ=0.94 min; MS (ESIpos): m/z=1133 (M+H)⁺.

### Intermediate F312

### N-[(2,5-Dioxo-2,5-dihydro-1H-pyrrol-1-yl)acetyl]-L-valyl-N-[(2S)-4-[{(1R)-1-[1-benzyl-4-(2,5-difluorophenyl)-1H-pyrrol-2-yl]-2,2-dimethylpropyl}(glycoloyl)amino]-1-{[2-(L-gamma-glutamylamino)ethyl]amino}-1-oxobutan-2-yl]-L-alaninamide/trifluoroacetic acid (1:1)

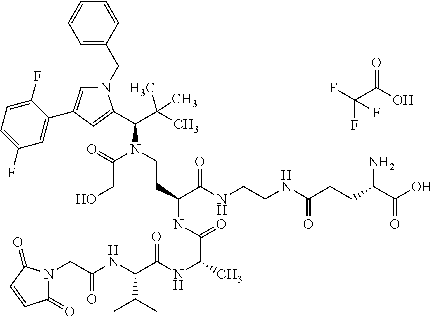 [SMILES: CC(C)[C@H](NC(=O)CN1C(=O)C=CC1=O)C(=O)N[C@@H](C)C(=O)N[C@@H](CCN(C(=O)CO)[C@@H](c1cc(-c2cc(F)ccc2F)cn1Cc1ccccc1)C(C)(C)C)C(=O)NCCNC(=O)CC[C@H](N)C(=O)O ; O=C(O)C(F)(F)F]

The title compound was prepared from intermediate C103 by coupling with N-[(benzyloxy)carbonyl]-L-valyl-L-alanine in the presence of HATU and N,N-diisopropylethylamine. In the next step, the Z protective group was removed by hydrogenating for 1 hour over 10% palladium on activated carbon in DCM/methanol 1:1 at RT under standard hydrogen pressure. The deprotected intermediate was then converted by coupling with (2,5-dioxo-2,5-dihydro-1H-pyrrol-1-ylacetic acid in the presence of HATU and N,N-diisopropylethylamine and finally by deprotection with zinc chloride and purification by preparative HPLC into the title compound.

LC-MS (Method 1): Rₜ=0.9 min; MS (ESIpos): m/z=992 (M+H)⁺.

### Intermediate F313

### S-[2-({(1R)-1-[1-Benzyl-4-(2,5-difluorophenyl)-1H-pyrrol-2-yl]-2,2-dimethylpropyl}{[(3R,4R)-4-fluoropyrrolidin-3-yl]methyl}amino)-2-oxoethyl]-N-[1-(2,5-dioxo-2,5-dihydro-1H-pyrrol-1-yl)-2,18-dioxo-6,9,12,15-tetraoxa-3-azaoctadecan-18-yl]-L-cysteine/trifluoroacetic acid (1:1)

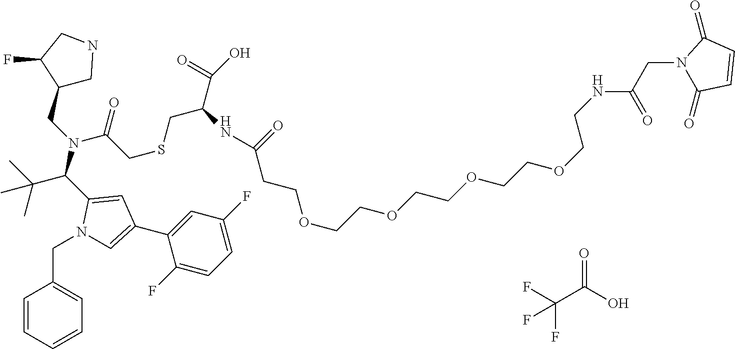 [SMILES: CC(C)(C)[C@H](c1cc(-c2cc(F)ccc2F)cn1Cc1ccccc1)N(C[C@@H]1CNC[C@@H]1F)C(=O)CSC[C@H](NC(=O)CCOCCOCCOCCOCCNC(=O)CN1C(=O)C=CC1=O)C(=O)O ; O=C(O)C(F)(F)F]

Under argon, 16.9 mg (0.13 mmol) of N,N-diisopropylethylamine and 50.0 mg (0.13 mmol) of HATU were added to a solution of 55.0 mg (0.14 mmol) of 1-(2,5-dioxo-2,5-dihydro-1H-pyrrol-1-yl)-2-oxo-6,9,12,15-tetraoxa-3-azaoctadecan-18-oic acid in 2.60 ml of DMF. The reaction mixture was stirred at RT for 10 minutes. A solution of 40.0 mg (0.05 mmol) of S-[2-({(1R)-1-[1-benzyl-4-(2,5-difluorophenyl)-1H-pyrrol-2-yl]-2,2-dimethylpropyl}{[(3R,4R)-4-fluoro-1-{[2-(trimethylsilyl)ethoxy]carbonyl}pyrrolidin-3-yl]methyl}amino)-2-oxoethyl]-L-cysteine (intermediate C107) was then added, and the mixture was stirred at RT overnight. Water was added and the mixture was extracted with dichloromethane. The organic phase was dried over magnesium sulphate and the solvent was evaporated under reduced pressure and the residue was dried under high vacuum. The residue was purified by prep. HPLC. This gave 10 mg (13% of theory, purity 82%) of the title compound.

LC-MS (Method 1): Rₜ=1.36 min; MS (ESIpos): m/z=1145 (M+H)⁺.

4.3 mg (0.03 mmol) of zinc chloride were added to a solution of 10.9 mg (7.8 mmol, 82% pure) of S-[2-({(1R)-1-[1-benzyl-4-(2,5-difluorophenyl)-1H-pyrrol-2-yl]-2,2-dimethylpropyl}{[(3R,4R)-4-fluoro-1-{[2-(trimethylsilyl)ethoxy]carbonyl}pyrrolidin-3-yl]methyl}amino)-2-oxoethyl]-N-[1-(2,5-dioxo-2,5-dihydro-1H-pyrrol-1-yl)-2,18-dioxo-6,9,12,15-tetraoxa-3-azaoctadecan-18-yl]-L-cysteine in 0.85 ml of 2,2,2-trifluoroethanol, and the reaction mixture was stirred at 50° C. for 2.5 h. 9.1 mg (0.03 mmol) of EDTA were then added, and the mixture was stirred for 15 minutes. The reaction mixture was purified by prep. HPLC. This gave 2.3 mg (26% of theory) of the title compound.

LC-MS (Method 1): Rₜ 0.89 min; MS (ESIpos): m/z=781 (M+H—CF₃CO₂H)⁺.

### Intermediate F314

### Trifluoroacetic acid/3-{[2-({(1R)-1-[1-benzyl-4-(2,5-difluorophenyl)-1H-pyrrol-2-yl]-2,2-dimethylpropyl}{[(3S,4R)-4-fluoropyrrolidin-3-yl]methyl}amino)-2-oxoethyl]sulphanyl}-N-(2-{[(2,5-dioxo-2,5-dihydro-1H-pyrrol-1-yl) acetyl]amino}ethylpropanamide

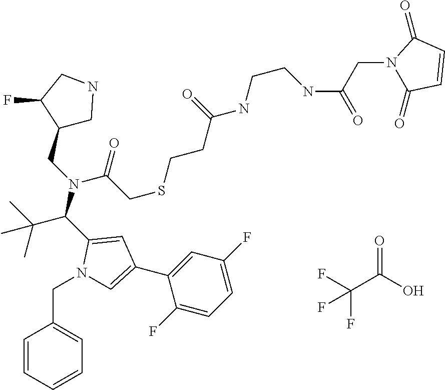 [SMILES: CC(C)(C)[C@H](c1cc(-c2cc(F)ccc2F)cn1Cc1ccccc1)N(C[C@@H]1CNC[C@@H]1F)C(=O)CSCCC(=O)NCCNC(=O)CN1C(=O)C=CC1=O ; O=C(O)C(F)(F)F]

Under argon, 16.89 mg (0.13 mmol) of N,N-diisopropylethylamine and 33.13 mg (0.087 mmol) of HATU were added to a solution of 50.0 mg (0.04 mmol) of 3-{[2-({(1R)-1-[1-benzyl-4-(2,5-difluorophenyl)-1H-pyrrol-2-yl]-2,2-dimethylpropyl}{[(3R,4R)-4-fluoro-1-{[2-(trimethylsilyl)ethoxy]carbonyl}pyrrolidin-3-yl]methyl}amino)-2-oxoethyl]sulphanyl}propanoic acid (intermediate C106) in 3.14 ml of DMF. The reaction mixture was stirred at RT for 10 minutes. A solution of 27.29 mg (0.09 mmol) of N-(2-aminoethyl)-2-(2,5-dioxo-2,5-dihydro-1H-pyrrol-1-yl)acetamide (1:1) trifluoroacetic acid (intermediate L1) was then added, and the mixture was stirred at RT for 15 minutes. Water was added and the mixture was extracted with dichloromethane. The organic phase was dried over magnesium sulphate and the solvent was evaporated under reduced pressure and the residue was dried under high vacuum. The residue was purified by prep. HPLC. This gave 41 mg (68% of theory, purity 66%) of the title compound.

LC-MS (Method 12): Rₜ=2.55 min; MS (ESIneg): m/z=959 (M−H+Na)⁻.

24.7 mg (0.18 mmol) of zinc chloride were added to a solution of 41.1 mg (0.03 mmol, purity 66%) of 2-(trimethylsilyl)ethyl-(3R,4R)-3-[2-{(1R)-1-[1-benzyl-4-(2,5-difluorophenyl)-1H-pyrrol-2-yl]-2,2-dimethylpropyl}-14-(2,5-dioxo-2,5-dihydro-1H-pyrrol-1-yl)-3,8,13-trioxo-5-thia-2,9,12-triazatetradec-1-yl]-4-fluoropyrrolidine-1-carboxylate in 2.54 ml of 2,2,2-trifluoroethanol, and the reaction mixture was stirred at 50° C. for 2.5 h. 53.0 mg (0.18 mmol) of EDTA were then added, and the mixture was stirred for 15 minutes. The reaction mixture was purified by prep. HPLC. This gave 10 mg (36% of theory) of the title compound.

LC-MS (Method 1): Rₜ 0.89 min; MS (ESIpos): m/z=781 (M+H—CF₃CO₂H)⁺.

### Intermediate F315

### S-{2-[(3-Aminopropyl){(1R)-1-[1-benzyl-4-(2,5-difluorophenyl)-1H-pyrrol-2-yl]-2,2-dimethylpropyl}amino]-2-oxoethyl}-N-{3-[5-(2-{[(2,5-dioxo-2,5-dihydro-1H-pyrrol-1-yl) acetyl]amino}ethyl)-1,2,4-oxadiazol-3-yl]propanoyl}-L-cysteine

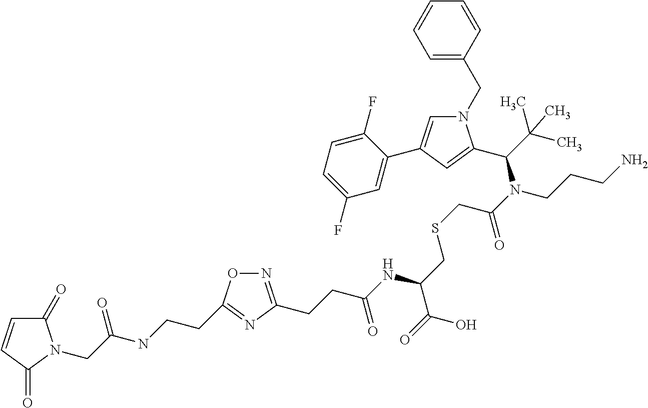 [SMILES: CC(C)(C)[C@H](c1cc(-c2cc(F)ccc2F)cn1Cc1ccccc1)N(CCCN)C(=O)CSC[C@H](NC(=O)CCc1noc(CCNC(=O)CN2C(=O)C=CC2=O)n1)C(=O)O]

Under argon, 18.02 mg (0.14 mmol) of N,N-diisopropylethylamine and 31.82 mg (0.09 mmol) of HATU were added to a solution of 50.0 mg (0.07 mmol) of 3-[5-(2-{[(2,5-dioxo-2,5-dihydro-1H-pyrrol-1-yl)acetyl]amino}ethyl)-1,2,4-oxadiazol-3-yl]propanoic acid (intermediate L100) in 3.5 ml of DMF. The reaction mixture was stirred at RT for 10 minutes. A solution of 50.0 mg (0.07 mmol) of N-(2-aminoethyl)-6-(2,5-dioxo-2,5-dihydro-1H-pyrrol-1-ylhexanamide acetate (1:1) (intermediate C107) was then added, and the mixture was stirred at RT for 2 h. Water was added and the mixture was extracted with dichloromethane. The organic phase was dried over magnesium sulphate and the solvent was evaporated under reduced pressure and the residue was dried under high vacuum. The residue was used further without purification. This gave 49 mg (21% of theory, purity 31%) of the title compound.

LC-MS (Method 1): Rₜ=1.30 min; MS (ESIpos): m/z=1022 (M+H)⁺.

8.0 mg (0.06 mmol) of zinc chloride were added to a solution of 49.0 mg (0.015 mmol, 31% pure) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-{3-[5-(2-{[(2,5-dioxo-2,5-dihydro-1H-pyrrol-1-ylacetyl]amino}ethyl)-1,2,4-oxadiazol-3-yl]propanoyl}-L-cysteine in 0.5 ml of 2,2,2-trifluoroethanol, and the reaction mixture was stirred at 50° C. for 2 h. 17.2 mg (0.06 mmol) of EDTA were then added, and the mixture was stirred for 15 minutes. The reaction mixture was purified by prep. HPLC. This gave 3 mg (21% of theory) of the title compound.

LC-MS (Method 1): Rₜ=0.91 min; MS (ESIpos): m/z=877 (M+H—CF₃CO₂H)⁺.

### Intermediate F316

### Trifluoroacetic acid/N-{2-[(3-{[2-({(1R)-1-[1-benzyl-4-(2,5-difluorophenyl)-1H-pyrrol-2-yl]-2,2-dimethylpropyl}{[(3S,4R)-4-fluoropyrrolidin-3-yl]methyl}amino)-2-oxoethyl]sulphanyl}propanoyl)amino]ethyl}-6-(2,5-dioxo-2,5-dihydro-1H-pyrrol-1-yl)hexan amide (1:1)

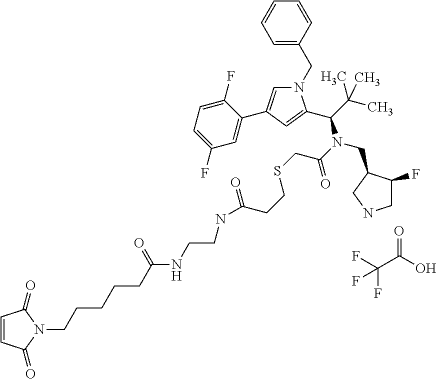

Under argon, 16.89 mg (0.13 mmol) of N,N-diisopropylethylamine and 33.13 mg (0.087 mmol) of HATU were added to a solution of 50.0 mg (0.04 mmol, 65% purity) of 3-{[2-({(1R)-1-[1-benzyl-4-(2,5-difluorophenyl)-1H-pyrrol-2-yl]-2,2-dimethylpropyl}{[(3R,4R)-4-fluoro-1-{[2-(trimethylsilyl)ethoxy]carbonyl}pyrrolidin-3-yl]methyl}amino)-2-oxoethyl]sulphanyl}propanoic acid (intermediate 106) in 3.0 ml of DMF. The reaction mixture was stirred at RT for 10 minutes. A solution of 37.2 mg (0.09 mmol, purity 70%) of N-(2-aminoethyl)-6-(2,5-dioxo-2,5-dihydro-1H-pyrrol-1-ylhexanamide acetate (1:1) (intermediate L73) was then added, and the mixture was stirred at RT for 7 minutes. Water was added and the mixture was extracted with dichloromethane. The organic phase was dried over magnesium sulphate and the solvent was evaporated under reduced pressure and the residue was dried under high vacuum. The residue was used further without purification. This gave 57 mg (77% of theory, purity 59%) of the title compound.

LC-MS (Method 12): Rₜ=2.60 min; MS (ESIpos): m/z=981 (M+H)⁺.

36.0 mg (0.27 mmol) of zinc chloride were added to a solution of 56.0 mg (0.03 mmol, 59% pure) of 2-(trimethylsilyl)ethyl (3R,4R)-3-[2-{(1R)-1-[1-benzyl-4-(2,5-difluorophenyl)-1H-pyrrol-2-yl]-2,2-dimethylpropyl}-18-(2,5-dioxo-2,5-dihydro-1H-pyrrol-1-yl)-3,8,13-trioxo-5-thia-2,9,12-triazaoctadec-1-yl]-4-fluoropyrrolidine-1-carboxylate in 2.8 ml of 2,2,2-trifluoroethanol, and the reaction mixture was stirred at 50° C. for 2 h. 78.3 mg (0.27 mmol) of EDTA were then added, and the mixture was stirred for 15 minutes. The reaction mixture was purified by prep. HPLC. This gave 16 mg (44% of theory, 85% pure) of the title compound.

LC-MS (Method 1): Rₜ=0.89 min; MS (ESIpos): m/z=837 (M+H-AcOH)⁺.

### Intermediate F317

### 1-[(S-{2-[(3-Aminopropyl){(1R)-1-[1-benzyl-4-(2,5-difluorophenyl)-1H-pyrrol-2-yl]-2,2-dimethylpropyl}amino]-2-oxoethyl}-N-[1-(2,5-dioxo-2,5-dihydro-1H-pyrrol-1-yl)-2,18-dioxo-6,9,12,15-tetraoxa-3-azaoctadecan-18-yl]-L-cysteinyl)amino]-3,6,9,12-tetraoxapentadecan-15-oic acid/trifluoro acetic acid (1:1)

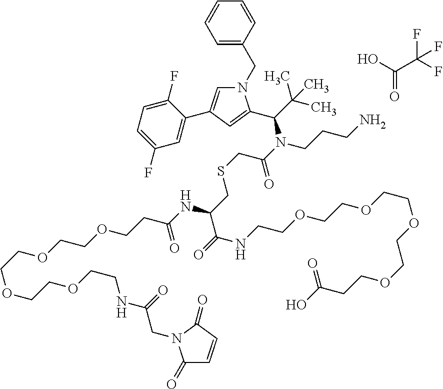 [SMILES: CC(C)(C)[C@H](c1cc(-c2cc(F)ccc2F)cn1Cc1ccccc1)N(CCCN)C(=O)CSC[C@H](NC(=O)CCOCCOCCOCCOCCNC(=O)CN1C(=O)C=CC1=O)C(=O)NCCOCCOCCOCCOCCC(=O)O ; O=C(O)C(F)(F)F]

Under argon, 30.2 mg (0.06 mmol) of N,N′-bis[(benzyloxy)carbonyl]-L-cystine were initially charged in 2.0 ml of water and 2.0 ml of isopropanol, and 56.7 mg (0.20 mmol) of TCEP were added. The reaction mixture was stirred at RT for 30 min. 50.0 mg (0.08 mmol) of 2-(trimethylsilyl)ethyl {3-[{(1R)-1-[1-benzyl-4-(2,5-difluorophenyl)-1H-pyrrol-2-yl]-2,2-dimethylpropyl}(chloroacetyl)amino]propyl}carbamate (intermediate C70), dissolved in 2.0 ml of isopropanol, and 122.2 mg (0.48 mmol) of 1,8-diazabicyclo[5.4.0]undec-7-ene were then added, and the reaction mixture was stirred at 50° C. for 7 h. Another 122.2 mg (0.48 mmol) of 1,8-diazabicyclo[5.4.0]undec-7-ene were then added, and the reaction mixture was stirred at 50° C. for 1 h. The mixture was diluted with ethyl acetate and the organic phase was extracted with water and sat. sodium bicarbonate solution and washed with sat. NaCl solution. The organic phase was dried over magnesium sulphate and the solvent was evaporated under reduced pressure. The residue was purified by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 43.1 mg (64% of theory) of the compound S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[(benzyloxy)carbonyl]-L-cysteine.

LC-MS (Method 1): Rₜ=1.46 min; MS (ESIpos): m/z=851 (M+H)⁺.

Under argon, S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[(benzyloxy)carbonyl]-L-cysteine (50.0 mg, 59 μmol) was dissolved in 1.0 ml of DMF, and N,N-diisopropylethylamine (20.5 μl, 117 μmol) and HATU (26.8 mg, 70 μmol) were added. The reaction mixture was stirred for 10 min. tert-Butyl 1-amino-3,6,9,12-tetraoxapentadecan-15-oate (22.6 mg, 70 μmol) was then added. The reaction mixture was stirred for 1 hour and then purified directly by prep. RP-HPLC (column: Reprosil 250×30; 10μ, flow rate: 50 ml/min, MeCN/water). The solvents were evaporated under reduced pressure and the residue was dried under high vacuum. This gave 59.3 mg (87.5% of theory) of the compound tert-butyl 1-({S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[(benzyloxy)carbonyl]-L-cysteinyl}amino)-3,6,9,12-tetraoxapentadecan-15-oate.

LC-MS (Method 12): Rt=2.97 min; MS (ESIpos): m/z=1154 [M+H]+

Under argon, palladium(II) acetate (6.74 mg, 30.0 μmol) was initially charged in 3.0 ml of dichloromethane, and triethylamine (13 μl, 90 μmol) and triethylsilane (96 μl, 600 μmol) were added. The reaction mixture was stirred for 5 min, and tert-butyl 1-({S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[(benzyloxy)carbonyl]-L-cysteinyl}amino)-3,6,9,12-tetraoxapentadecan-15-oate (69.3 mg, 60.0 μmol) in 1.0 ml of dichloromethane was then added. The reaction mixture was stirred at RT for 2 hours, and triethylsilane (48 μl, 300 μmol) was then added. The reaction mixture was stirred at RT for 2 hours, and 2.0 ml of water (0.1% TFA) were added. The solvent was evaporated under reduced pressure without heating. The residue was taken up in acetonitrile, filtered through a syringe filter and purified by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 65.9 mg (97% of theory) of the compound trifluoroacetic acid/tert-butyl 1-{[S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteinyl]amino}-3,6,9,12-tetraoxapentadecan-15-oate (1:1).

LC-MS (Method 1): Rₜ=1.22 min; MS (ESIpos): m/z=1020 [M+H]⁺

Under argon, 1-(2,5-dioxo-2,5-dihydro-1H-pyrrol-1-yl)-2-oxo-6,9,12,15-tetraoxa-3-azaoctadecan-18-oic acid (4.26 mg, 10.6 μmol) was initially charged in 1.0 ml of DMF, and N,N-diisopropylethylamine (3.2 μl, 18 μmol) and HATU (4.02 mg, 10.6 μmol) were added. The reaction mixture was stirred for 10 min, and trifluoroacetic acid/tert-butyl 1-{[S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteinyl]amino}-3,6,9,12-tetraoxapentadecan-15-oate (1:1) (10.0 mg, 8.82 μmol), dissolved in 1.0 ml of DMF and N,N-diisopropylethylamine (1.5 μl, 8.8 μmol), was then added. The reaction mixture was stirred at RT for 1 hour and purified directly by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 10.9 mg (93% of theory) of the compound tert-butyl 1-({S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[1-(2,5-dioxo-2,5-dihydro-1H-pyrrol-1-yl)-2,18-dioxo-6,9,12,15-tetraoxa-3-azaoctadecan-18-yl]-L-cysteinyl}amino)-3,6,9,12-tetraoxapentadecan-15-oate.

LC-MS (Method 1): Rₜ=1.44 min; MS (ESIpos): m/z=1404 [M+H]⁺

tert-Butyl 1-({S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[1-(2,5-dioxo-2,5-dihydro-1H-pyrrol-1-yl)-2,18-dioxo-6,9,12,15-tetraoxa-3-azaoctadecan-18-yl]-L-cysteinyl}amino)-3,6,9,12-tetraoxapentadecan-15-oate (8.20 mg, 5.84 μmol) was dissolved in 2.0 trifluoroethanol, and zinc chloride (4.77 mg, 35.0 μmol) was added. The reaction mixture was stirred at 50° C. for 1 hour.

Zinc chloride (4.77 mg, 35.0 μmol) was added and the reaction mixture was stirred at 50° C. for 1 hour. Ethylenediamine-N,N,N′,N′-tetraacetic acid (10.2 mg, 35.0 μmol) was added, the reaction mixture was stirred for 10 min and water (0.1% TFA) was then added. Purification was carried out directly by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 4.1 mg (53% of theory) of the title compound.

LC-MS (Method 1): Rₜ=0.90 min; MS (ESIpos): m/z=1204 [M+H]⁺

### Intermediate F318

### Trifluoroacetic acid/3-{[2-([3-amino-4-fluorobutyl]{(1R)-1-[1-benzyl-4-(2,5-difluorophenyl)-1H-pyrrol-2-yl]-2,2-dimethylpropyl}amino)-2-oxoethyl]sulphanyl}-N-(2-{[(2,5-dioxo-2,5-dihydro-1H-pyrrol-1-ylacetyl]amino}ethylpropanamide (1:1)

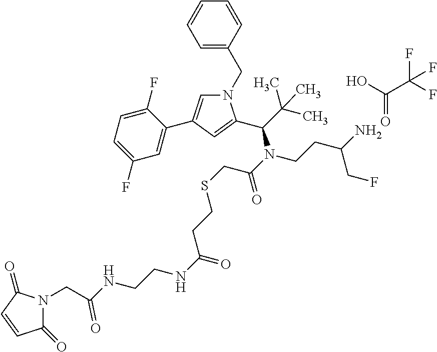

(1R)-1-[1-Benzyl-4-(2,5-difluorophenyl)-1H-pyrrol-2-yl]-2,2-dimethylpropane-1-amine (124 mg, 350 μmol) (intermediate C52) was initially charged in 5.0 ml of dichloromethane, and sodium triacetoxyborohydride (104 mg, 491 μmol) and acetic acid (23 μl, 400 μmol) were added. The reaction mixture was stirred at RT for 5 min, and tert-butyl [l-fluoro-4-oxobutan-2-yl]carbamate (82.7 mg, 403 μmol) (intermediate L123), dissolved in 3.0 ml of dichloromethane, was then added. The reaction mixture was stirred at RT overnight, and ethyl acetate was then added. The mixture was washed twice with saturated sodium bicarbonate solution and then with saturated sodium chloride solution. The organic phase was dried over magnesium sulphate and then concentrated. The residue was purified by column chromatography on Biotage/Isolera (SNAP 25 g) using the mobile phase cyclohexane/ethyl acetate 95:5. The solvents were evaporated under reduced pressure and the residue was dried under high vacuum. This gave 146 mg (77% of theory) of the compound tert-butyl [4-({(1R)-1-[1-benzyl-4-(2,5-difluorophenyl)-1H-pyrrol-2-yl]-2,2-dimethylpropyl}amino)-1-fluorobutan-2-yl]carbamate.

LC-MS (Method 13): Rₜ=2.57 min; MS (ESIneg): m/z=588 [M+CHOOH—H]⁻

tert-Butyl [4-({(1R)-1-[1-benzyl-4-(2,5-difluorophenyl)-1H-pyrrol-2-yl]-2,2-dimethylpropyl}amino)-1-fluorobutan-2-yl]carbamate (100 mg, 184 μmol) was dissolved in 6.0 ml of DCM, and triethylamine (85 μl, 610 μmol) and chloroacetyl chloride (47 μl, 590 μmol) were added at 0° C. The reaction mixture was stirred at RT overnight. The solvent was evaporated under reduced pressure. The residue was taken up in acetonitrile/water and purified by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 80 mg (70% of theory) of the compound tert-butyl-{4-[{(1R)-1-[1-benzyl-4-(2,5-difluorophenyl)-1H-pyrrol-2-yl]-2,2-dimethylpropyl}(chloroacetyl)amino]-1-fluorobutan-2-yl}carbamate.

LC-MS (Method 12): Rₜ=2.67 min; MS (ESIneg): m/z=664 [M−H+COOH]⁻

tert-Butyl {4-[{(1R)-1-[1-benzyl-4-(2,5-difluorophenyl)-1H-pyrrol-2-yl]-2,2-dimethylpropyl}(chloroacetyl)amino]-1-fluorobutan-2-yl}carbamate (79.2 mg, 128 μmol) and 3-sulphanylpropanoic acid (12 μl, 140 μmol) were initially charged in 3.0 ml of methanol with a drop of water. Potassium carbonate (61.8 mg, 447 μmol) was added and the reaction mixture was stirred at 50° C. for 4 h. Ethyl acetate was added and the mixture was washed repeatedly with water. The organic phase was washed with saturated sodium chloride solution, dried over magnesium sulphate and then concentrated. The residue was purified by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 68.6 mg (78% of theory) of the compound 9-{(1R)-1-[1-benzyl-4-(2,5-difluorophenyl)-1H-pyrrol-2-yl]-2,2-dimethylpropyl}-6-(fluoromethyl)-2,2-dimethyl-4,10-dioxo-3-oxa-12-thia-5,9-diazapentadecan-15-oic acid.

LC-MS (Method 12): Rₜ=2.46 min; MS (ESIneg): m/z=688 [M−H]⁻

9-{(1R)-1-[1-Benzyl-4-(2,5-difluorophenyl)-1H-pyrrol-2-yl]-2,2-dimethylpropyl}-6-(fluoromethyl)-2,2-dimethyl-4,10-dioxo-3-oxa-12-thia-5,9-diazapentadecan-15-oic acid (15.0 mg, 21.7 μmol) and trifluoroacetic acid/N-(2-aminoethyl)-2-(2,5-dioxo-2,5-dihydro-1H-pyrrol-1-yl)acetamide (1:1) (8.12 mg, 26.1 μmol) (intermediate L1) were initially charged in 1.6 ml of DMF. HATU (9.92 mg, 26.1 μmol) and N,N-diisopropylethylamine (11 μl, 65 μmol) were added and the reaction mixture was stirred at RT for 5 min. Water (0.1% TFA) was added and the reaction mixture was purified directly by prep. RP-HPLC (column: Reprosil 250×30; 10μ, flow rate: 50 ml/min, MeCN/water). The solvents were evaporated under reduced pressure and the residue was dried under high vacuum. This gave 18.6 mg (98% of theory) of the compound tert-butyl [13-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-17-fluoro-2,7,12-trioxo-10-thia-3,6,13-triazaheptadecan-16-yl]carbamate.

LC-MS (Method 12): Rₜ=2.36 min; MS (ESIpos): m/z=869 [M+H]⁺

tert-Butyl [13-{(1R)-1-[1-benzyl-4-(2,5-difluorophenyl)-1H-pyrrol-2-yl]-2,2-dimethylpropyl}-1-(2,5-dioxo-2,5-dihydro-1H-pyrrol-1-yl)-17-fluoro-2,7,12-trioxo-10-thia-3,6,13-triazaheptadecan-16-yl]carbamate (17.0 mg, 19.6 μmol) was dissolved in 2.0 ml of trifluoroethanol. Zinc chloride (16.0 mg, 117 μmol) was added and the reaction mixture was stirred at 50° C. for 1 hour. Once more, zinc chloride (16.0 mg, 117 μmol) was added and the reaction mixture was stirred at 50° C. for 1 hour. Ethylenediamine-N,N,N′,N′-tetraacetic acid (68.6 mg, 234 μmol) was added to the mixture, water (0.1% TFA) was then added and the mixture was subsequently concentrated under reduced pressure. The residue was purified by prep. RP-HPLC (column: Reprosil 250×30; 10μ, flow rate: 50 ml/min, MeCN/water, 0.1% TFA). The solvents were evaporated under reduced pressure and the residue was taken up in a little water and lyophilized. This gave 10.7 mg (60% of theory) of the title compound.

LC-MS (Method 14): Rₜ=5.51 min; MS (ESIpos): m/z=769 [M+H]⁺

### Intermediate F319

### N-(3-{[2-({(1R)-1-[1-Benzyl-4-(2,5-difluorophenyl)-1H-pyrrol-2-yl]-2,2-dimethylpropyl}[3-({N-[6-(2,5-dioxo-2,5-dihydro-1H-pyrrol-1-yl)hexanoyl]-L-valyl-L-alanyl}aminopropyl]amino)-2-oxoethyl]sulphanyl}propanoyl)-beta-alanyl-L-aspartic acid

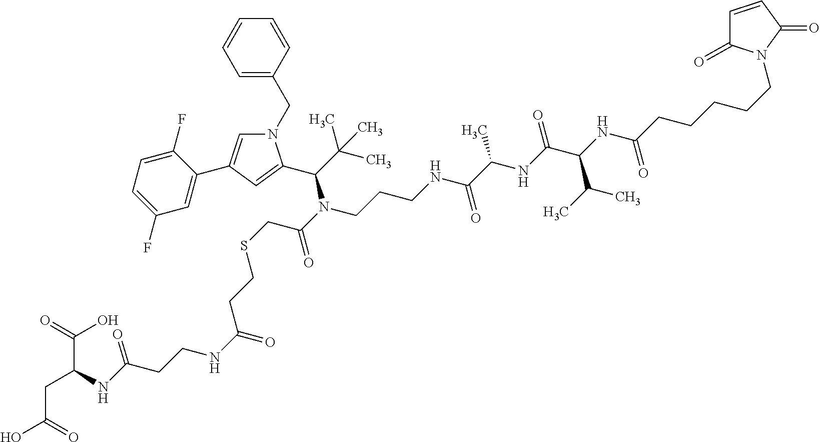 [SMILES: CC(C)[C@H](NC(=O)CCCCCN1C(=O)C=CC1=O)C(=O)N[C@@H](C)C(=O)NCCCN(C(=O)CSCCC(=O)NCCC(=O)N[C@@H](CC(=O)O)C(=O)O)[C@@H](c1cc(-c2cc(F)ccc2F)cn1Cc1ccccc1)C(C)(C)C]

Under argon, N-[6-(2,5-dioxo-2,5-dihydro-1H-pyrrol-1-yl)hexanoyl]-L-valyl-N-[3-({(1R)-1-[1-benzyl-4-(2,5-difluorophenyl)-1H-pyrrol-2-yl]-2,2-dimethylpropyl}[({3-[(2-carboxyethyl)amino]-3-oxopropyl}sulphanyl)acetyl]amino)propyl]-L-alaninamide (9.80 mg, 9.88 μmol) (intermediate 016) and di-tert-butyl L-aspartate hydrochloride (1:1) (3.34 mg, 11.9 μmol) were initially charged in 1.0 ml of DMF, and HATU (4.51 mg, 11.9 μmol) and N,N-diisopropylethylamine (5.2 μl, 30 μmol) were added. The reaction mixture was stirred at RT for 10 min and purified directly by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 10.5 mg (87% of theory) of the compound di-tert-butyl N-(3-{[2-({(1R)-1-[1-benzyl-4-(2,5-difluorophenyl)-1H-pyrrol-2-yl]-2,2-dimethylpropyl}[3-({N-[6-(2,5-dioxo-2,5-dihydro-1H-pyrrol-1-yl)hexanoyl]-L-valyl-L- alanyl}aminopropyl]amino)-2-oxoethyl]sulphanyl}propanoyl)-beta-alanyl-L-aspartate.

LC-MS (Method 1): Rₜ=1.33 min; MS (ESIpos): m/z=1219 [M+H]⁺

Di-tert-butyl N-(3-{[2-({(1R)-1-[1-benzyl-4-(2,5-difluorophenyl)-1H-pyrrol-2-yl]-2,2-dimethylpropyl}[3-({N-[6-(2,5-dioxo-2,5-dihydro-1H-pyrrol-1-yl)hexanoyl]-L-valyl-L-alanyl}amino)propyl]amino)-2-oxoethyl]sulphanyl}propanoyl)-beta-alanyl-L-aspartate (9.70 mg, 7.95 μmol) was dissolved in 1.5 ml of trifluoroethanol. Zinc chloride (6.50 mg, 47.7 μmol) was added and the reaction mixture was stirred at 50° C. for 1 hour. Once more, zinc chloride (6.50 mg, 47.7 μmol) was added and the reaction mixture was stirred at 50° C. for 1 hour. Ethylenediamine-N,N,N′,N′-tetraacetic acid (27.9 mg, 55.4 μmol) was added to the mixture, water (0.1% TFA) was then added and the mixture was subsequently concentrated under reduced pressure. The residue was purified by prep. RP-HPLC (column: Reprosil 250×30; 10μ, flow rate: 50 ml/min, MeCN/water, 0.1% TFA). The solvents were evaporated under reduced pressure and the residue was taken up in a little water and lyophilized. This gave 4.10 mg (47% of theory) of the title compound.

LC-MS (Method 1): Rₜ=1.07 min; MS (ESIpos): m/z=1107 [M+H]⁺

### Intermediate F320

### N-[6-(2,5-Dioxo-2,5-dihydro-1H-pyrrol-1-yl)hexanoyl]-L-valyl-N-[3-({(1R)-1-[1-benzyl-4-(2,5-difluorophenyl)-1H-pyrrol-2-yl]-2,2-dimethylpropyl}{[(3-{[(1S)-1,3-dicarboxypropyl]amino}-3-oxopropyl) sulphanyl]acetyl}aminopropyl]-L-alaninamide

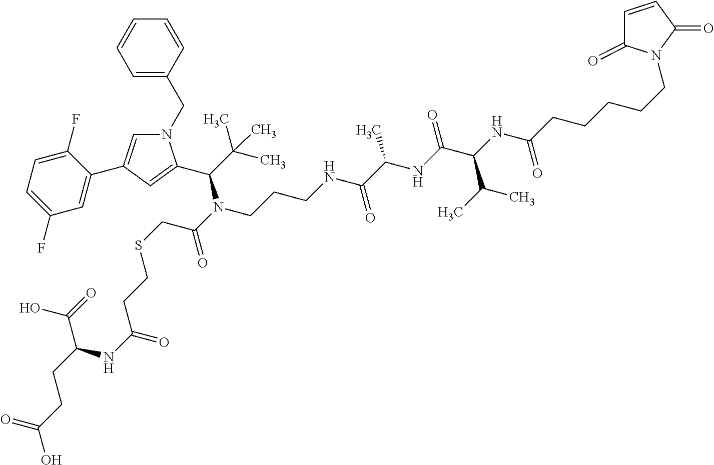 [SMILES: CC(C)[C@H](NC(=O)CCCCCN1C(=O)C=CC1=O)C(=O)N[C@@H](C)C(=O)NCCCN(C(=O)CSCCC(=O)N[C@@H](CCC(=O)O)C(=O)O)[C@@H](c1cc(-c2cc(F)ccc2F)cn1Cc1ccccc1)C(C)(C)C]

Under argon, N-[6-(2,5-dioxo-2,5-dihydro-1H-pyrrol-1-yl)hexanoyl]-L-valyl-N-[3-({(1R)-1-[1-benzyl-4-(2,5-difluorophenyl)-1H-pyrrol-2-yl]-2,2-dimethylpropyl}{[(2-carboxyethyl)sulphanyl]acetyl}amino)propyl]-L-alaninamide (20.0 mg, 21.7 μmol) (intermediate C115) and di-tert-butyl L-glutamate hydrochloride (1:1) (7.71 mg, 26.1 μmol) were initially charged in 2.0 ml of DMF, and HATU (9.91 mg, 26.1 μmol) and N,N-diisopropylethylamine (11 μl, 65 μmol) were added. The reaction mixture was stirred at RT for 10 min and then purified directly by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 16.4 mg (65% of theory) of the compound di-tert-butyl (2S)-2-{[(13S,16S)-7-{(1R)-1-[1-benzyl-4-(2,5-difluorophenyl)-1H-pyrrol-2-yl]-2,2-dimethylpropyl}-23-(2,5-dioxo-2,5-dihydro-1H-pyrrol-1-yl)-16-isopropyl-13-methyl-6,12,15,18-tetraoxo-4-thia-7,11,14,17-tetraazatricosan-1-oyl]amino}pentanedioate.

LC-MS (Method 1): Rₜ=1.40 min; MS (ESIpos): m/z=1162 [M+H]⁺

Di-tert-butyl (2S)-2-{[(13S,16S)-7-{(1R)-1-[1-benzyl-4-(2,5-difluorophenyl)-1H-pyrrol-2-yl]-2,2-dimethylpropyl}-23-(2,5-dioxo-2,5-dihydro-1H-pyrrol-1-yl)-16-isopropyl-13-methyl-6,12,15,18-tetraoxo-4-thia-7,11,14,17-tetraazatricosan-1-oyl]amino}pentanedioate (14.7 mg, 12.6 μmol) was dissolved in 1.5 ml of trifluoroethanol. Zinc chloride (10.3 mg, 75.9 μmol) was added and the reaction mixture was stirred at 50° C. for 1 hour. Once more, zinc chloride (10.3 mg, 75.9 μmol) was added and the reaction mixture was stirred at 50° C. for 1 hour. Ethylenediamine-N,N,N′,N′-tetraacetic acid (44.4 mg, 152 μmol) was added to the mixture, water (0.1% TFA) was then added and the mixture was subsequently concentrated under reduced pressure. The residue was purified by prep. RP-HPLC (column: Reprosil 250×30; 10μ, flow rate: 50 ml/min, MeCN/water, 0.1% TFA). The solvents were evaporated under reduced pressure and the residue was taken up in a little water and lyophilized. This gave 6.0 mg (45% of theory) of the title compound.

LC-MS (Method 1): Rₜ=1.10 min; MS (ESIneg): m/z=1048 [M−H]⁻

### Intermediate F321

### N-(3-{[2-({(1R)-1-[1-Benzyl-4-(2,5-difluorophenyl)-1H-pyrrol-2-yl]-2,2-dimethylpropyl}[3-({N-[6-(2,5-dioxo-2,5-dihydro-1H-pyrrol-1-yl)hexanoyl]-L-valyl-L-alanyl}aminopropyl]amino)-2-oxoethyl]sulphanyl}propanoyl)-beta-alanyl-D-glutamic acid

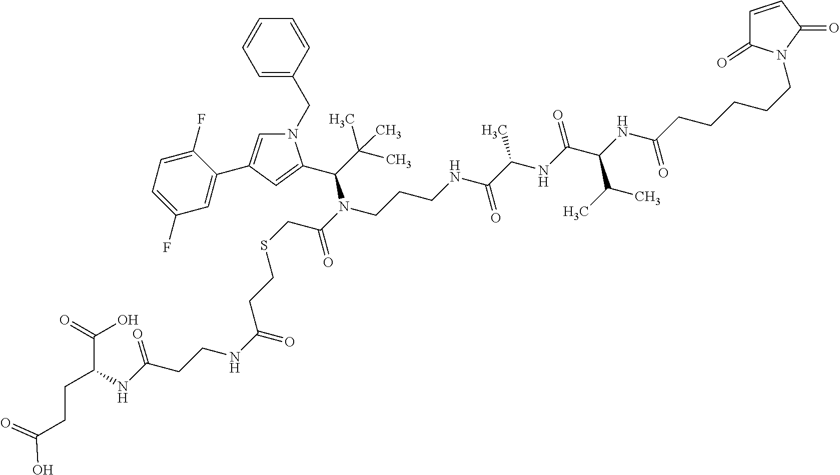 [SMILES: CC(C)[C@H](NC(=O)CCCCCN1C(=O)C=CC1=O)C(=O)N[C@@H](C)C(=O)NCCCN(C(=O)CSCCC(=O)NCCC(=O)N[C@H](CCC(=O)O)C(=O)O)[C@@H](c1cc(-c2cc(F)ccc2F)cn1Cc1ccccc1)C(C)(C)C]

Under argon, N-[6-(2,5-dioxo-2,5-dihydro-1H-pyrrol-1-yl)hexanoyl]-L-valyl-N-[3-({(1R)-1-[1-benzyl-4-(2,5-difluorophenyl)-1H-pyrrol-2-yl]-2,2-dimethylpropyl}[({3-[(2-carboxyethyl)amino]-3-oxopropyl}sulphanyl)acetyl]amino)propyl]-L-alaninamide (9.80 mg, 9.88 μmol) (intermediate 016) and di-tert-butyl D-glutamate hydrochloride (1:1) (3.51 mg, 11.9 μmol) were initially charged in 1.0 ml of DMF, and HATU (4.51 mg, 11.9 μmol) and N,N-diisopropylethylamine (5.2 μl, 30 μmol) were added. The reaction mixture was stirred at RT for 10 min and purified directly by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 11.7 mg (96% of theory) of the compound di-tert-butyl N-(3-{[2-({(1R)-1-[1-benzyl-4-(2,5-difluorophenyl)-1H-pyrrol-2-yl]-2,2-dimethylpropyl}[3-({N-[6-(2,5-dioxo-2,5-dihydro-1H-pyrrol-1-yl)hexanoyl]-L-valyl-L- alanyl}amino)propy 1]amino)-2-oxoethyl]sulphanyl}propanoyl)-beta-alanyl-D-glutamate.

LC-MS (Method 1): Rₜ=1.34 min; MS (ESIpos): m/z=1233 [M+H]⁺

Di-tert-butyl N-(3-{[2-({(1R)-1-[1-benzyl-4-(2,5-difluorophenyl)-1H-pyrrol-2-yl]-2,2-dimethylpropyl}[3-({N-[6-(2,5-dioxo-2,5-dihydro-1H-pyrrol-1-yl)hexanoyl]-L-valyl-L-alanyl}amino)propyl]amino)-2-oxoethyl]sulphanyl}propanoyl)-beta-alanyl-D-glutamate (11.5 mg, 9.32 μmol) was dissolved in 1.5 ml of trifluoroethanol. (7.62 mg, 55.9 μmol) was added and the reaction mixture was stirred at 50° C. for 1 hour. Once more, zinc chloride (7.62 mg, 55.9 μmol) was added and the reaction mixture was stirred at 50° C. for 1 hour. Ethylenediamine-N,N,N′,N′-tetraacetic acid (32.6 mg, 112 μmol) was added to the mixture, water (0.1% TFA) was then added and the mixture was subsequently concentrated under reduced pressure. The residue was purified by prep. RP-HPLC (column: Reprosil 250×30; 10μ, flow rate: 50 ml/min, MeCN/water, 0.1% TFA). The solvents were evaporated under reduced pressure and the residue was taken up in a little water and lyophilized. This gave 6.5 mg (62% of theory) of the title compound.

LC-MS (Method 1): Rₜ=1.07 min; MS (ESIpos): m/z=1121 [M+H]⁺

### Intermediate F322

### N-(3-{[2-({(1R)-1-[1-Benzyl-4-(2,5-difluorophenyl)-1H-pyrrol-2-yl]-2,2-dimethylpropyl}[3-({N-[6-(2,5-dioxo-2,5-dihydro-1H-pyrrol-1-yl)hexanoyl]-L-valyl-L-alanyl}aminopropyl]amino)-2-oxoethyl]sulphanyl}propanoyl)-beta-alanyl-L-glutamic acid

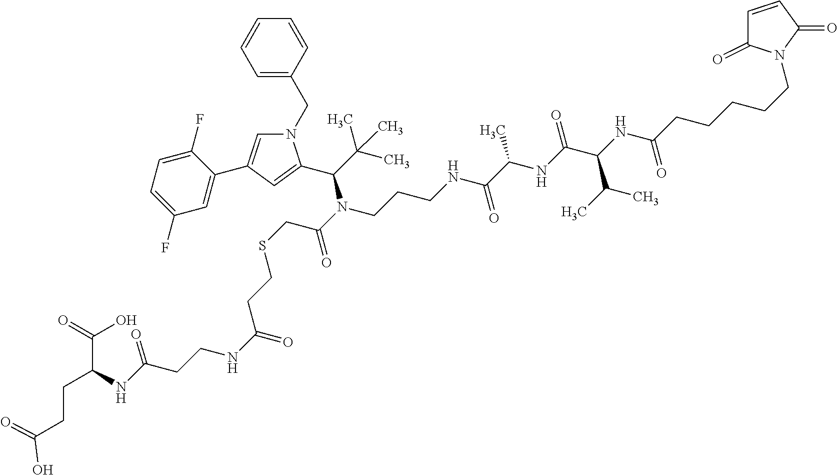 [SMILES: CC(C)[C@H](NC(=O)CCCCCN1C(=O)C=CC1=O)C(=O)N[C@@H](C)C(=O)NCCCN(C(=O)CSCCC(=O)NCCC(=O)N[C@@H](CCC(=O)O)C(=O)O)[C@@H](c1cc(-c2cc(F)ccc2F)cn1Cc1ccccc1)C(C)(C)C]

Under argon, N-[6-(2,5-dioxo-2,5-dihydro-1H-pyrrol-1-yl)hexanoyl]-L-valyl-N-[3-({(1R)-1-[1-benzyl-4-(2,5-difluorophenyl)-1H-pyrrol-2-yl]-2,2-dimethylpropyl}[({3-[(2-carboxyethyl)amino]-3-oxopropyl}sulphanyl)acetyl]amino)propyl]-L-alaninamide (9.80 mg, 9.88 μmol) (intermediate C116) and di-tert-butyl L-glutamate hydrochloride (1:1) (3.51 mg, 11.9 μmol) were initially charged in 1.0 ml of DMF, and HATU (4.51 mg, 11.9 μmol) and N,N-diisopropylethylamine (5.2 μl, 30 μmol) were added. The reaction mixture was stirred at RT for 10 min and purified directly by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 11.3 mg (93% of theory) of the compound of di-tert-butyl N-(3-{[2-({(1R)-1-[1-benzyl-4-(2,5-difluorophenyl)-1H-pyrrol-2-yl]-2,2-dimethylpropyl}[3-({N-[6-(2,5-dioxo-2,5-dihydro-1H-pyrrol-1-yl)hexanoyl]-L-valyl-L-alanyl}aminopropyl]amino)-2-oxoethyl]sulphanyl}propanoyl)-beta-alanyl-L-glutamate.

LC-MS (Method 1): Rₜ=1.34 min; MS (ESIpos): m/z=1233 [M+H]⁺

Di-tert-butyl N-(3-{[2-({(1R)-1-[1-benzyl-4-(2,5-difluorophenyl)-1H-pyrrol-2-yl]-2,2-dimethylpropyl}[3-({N-[6-(2,5-dioxo-2,5-dihydro-1H-pyrrol-1-yl)hexanoyl]-L-valyl-L-alanyl}amino)propyl]amino)-2-oxoethyl]sulphanyl}propanoyl)-beta-alanyl-L-glutamate (11.0 mg, 8.92 μmol) was dissolved in 1.5 ml of trifluoroethanol. Zinc chloride (7.29 mg, 53.5 μmol) was added and the reaction mixture was stirred at 50° C. for 1 hour. Once more, zinc chloride (7.29 mg, 53.5 μmol) was added and the reaction mixture was stirred at 50° C. for 1 hour. Ethylenediamine-N,N,N′,N′-tetraacetic acid (31.2 mg, 107 μmol) was added to the mixture, water (0.1% TFA) was then added and the mixture was subsequently concentrated under reduced pressure. The residue was purified by prep. RP-HPLC (column: Reprosil 250×30; 10μ, flow rate: 50 ml/min, MeCN/water, 0.1% TFA). The solvents were evaporated under reduced pressure and the residue was taken up in a little water and lyophilized. This gave 5.10 mg (51% of theory) of the title compound.

LC-MS (Method 1): Rₜ=1.07 min; MS (ESIpos): m/z=1121 [M+H]⁺

### Intermediate F323

### N-[6-(2,5-Dioxo-2,5-dihydro-1H-pyrrol-1-yl)hexanoyl]-L-valyl-N-[3-({[(3-{[(1R)-2-(L-beta-asparagylamino)-1-carboxyethyl]amino}-3-oxopropyl) sulphanyl]acetyl}{(1R)-1-[1-benzyl-4-(2,5-difluorophenyl)-1H-pyrrol-2-yl]-2,2-dimethylpropyl}amino)propyl]-L-alaninamide/trifluoroacetic acid (1:1)

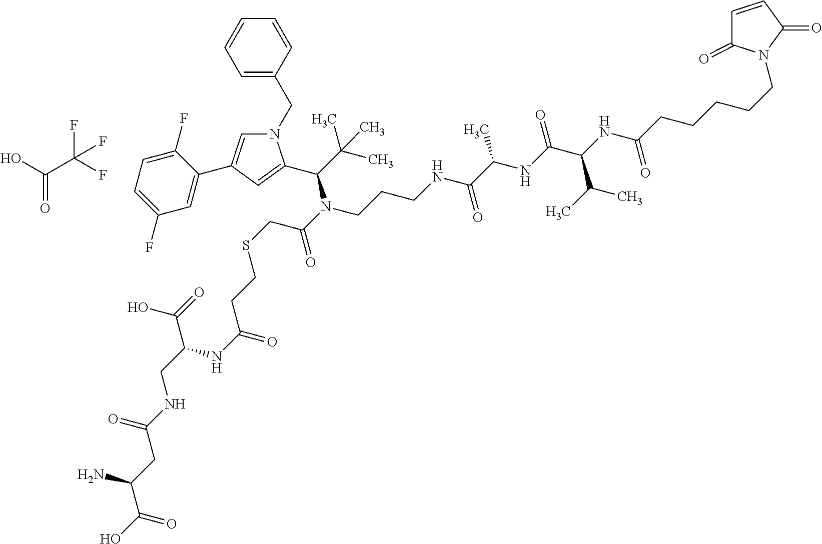

Under argon, N-[6-(2,5-dioxo-2,5-dihydro-1H-pyrrol-1-yl)hexanoyl]-L-valyl-N-[3-({(1R)-1-[1-benzyl-4-(2,5-difluorophenyl)-1H-pyrrol-2-yl]-2,2-dimethylpropyl}{[(2-carboxyethyl)sulphanyl]acetyl}amino)propyl]-L-alaninamide (10.0 mg, 7.05 μmol) (intermediate 015) and tert-butyl N-{(2R)-2-amino-3-oxo-3-[2-(trimethylsilyl)ethoxy]propyl}-N²-(tert-butoxycarbonyl)-L-aspartate (4.02 mg, 8.46 μmol) (intermediate L124) were initially charged in 2.0 ml of DMF, and HATU (3.22 mg, 8.46 μmol) and N,N-diisopropylethylamine (3.7 μl, 21 μmol) were added. The reaction mixture was stirred at RT for 10 min and purified directly by prep. RP-HPLC (column: Reprosil 250×30; 10μ, flow rate: 50 ml/min, MeCN/water, 0.1% TFA). The solvents were evaporated under reduced pressure and the residue was taken up in a little water and lyophilized. This gave 4.3 mg (32% of theory) of the compound 6-tert-butyl 11-[2-(trimethylsilyl)ethyl](6S,11R,25S,28S)-19-{(1R)-1-[1-benzyl-4-(2,5-difluorophenyl)-1H-pyrrol-2-yl]-2,2-dimethylpropyl}-35-(2,5-dioxo-2,5-dihydro-1H-pyrrol-1-yl)-28-isopropyl-2,2,25-trimethyl-4,8,13,18,24,27,30-heptaoxo-3-oxa-16-thia-5,9,12,19,23,26,29-heptaazapentatriacontane-6,11-dicarboxylate.

LC-MS (Method 5): Rₜ=5.32 min; MS (ESIpos): m/z=1379 [M+H]⁺

6-tert-Butyl 11-[2-(trimethylsilylethyl](6S,11R,25S,28S)-19-{(1R)-1-[1-benzyl-4-(2,5-difluorophenyl)-1H-pyrrol-2-yl]-2,2-dimethylpropyl}-35-(2,5-dioxo-2,5-dihydro-1H-pyrrol-1-yl)-28-isopropyl-2,2,25-trimethyl-4,8,13,18,24,27,30-heptaoxo-3-oxa-16-thia-5,9,12,19,23,26,29-heptaazapentatriacontane-6,11-dicarboxylate (4.10 mg, 73% pure, 2.17 μmol) was dissolved in 2.0 ml of trifluoroethanol. Zinc chloride (1.77 mg, 13.0 μmol) was added and the reaction mixture was stirred at 50° C. for 1 hour. Five more times, zinc chloride (1.77 mg, 13.0 μmol) was added and the reaction mixture was stirred at 50° C. for 1 hour. Ethylenedi amine-N,N,N′,N′-tetraacetic acid (22.0 mg, 78 μmol) was added to the mixture, water (0.1% TFA) was then added and the mixture was subsequently concentrated under reduced pressure. The residue was purified by prep. RP-HPLC (column: Reprosil 250×30; 10μ, flow rate: 50 ml/min, MeCN/water, 0.1% TFA). The solvents were evaporated under reduced pressure and the residue was taken up in a little water and lyophilized. This gave 2.1 mg (69% of theory) of the title compound.

LC-MS (Method 1): Rₜ=0.98 min; MS (ESIpos): m/z=1122 [M+H]⁺

### Intermediate F324

### S-[2-({(1R)-1-[1-Benzyl-4-(2,5-difluorophenyl)-1H-pyrrol-2-yl]-2,2-dimethylpropyl}[pyrrolidin-3-ylmethyl]amino)-2-oxoethyl]-N-[1-(2,5-dioxo-2,5-dihydro-1H-pyrrol-1-yl)-2,18-dioxo-6,9,12,15-tetraoxa-3-azaoctadecan-18-yl]-L-cysteine/trifluoroacetic acid (1:1) (isomer 1)

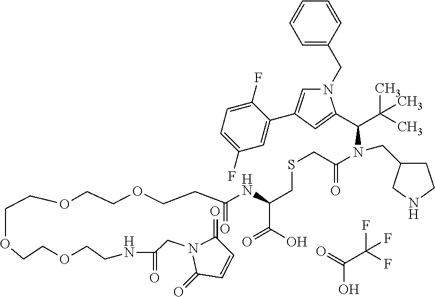

Under argon, 1-(2,5-dioxo-2,5-dihydro-1H-pyrrol-1-yl)-2-oxo-6,9,12,15-tetraoxa-3-azaoctadecan-18-oic acid (99.6 mg, 247 μmol) (intermediate L74) was initially charged in 1.4 ml of DMF, and HATU (90.4 mg, 238 μmol) and N,N-diisopropylethylamine (41 μl, 240 μmol) were added. The reaction mixture was stirred at RT for 10 min, and S-[2-({(1R)-1-[1-benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]-L-cysteine (70.0 mg, 95.2 μmol) (intermediate C90), dissolved in 1.4 ml of DMF, was added. The reaction mixture was stirred at RT overnight and purified directly by prep. RP-HPLC (column: Reprosil 250×30; 10μ, flow rate: 50 ml/min, MeCN/water, 0.1% TFA). The solvents were evaporated under reduced pressure and the residue was taken up in a little water and lyophilized. This gave 19.0 mg (18.4% of theory) of the compound S-[2-({(1R)-1-[1-benzyl-4-(2,5-difluorophenyl)-1H-pyrrol-2-yl]-2,2-dimethylpropyl}{[(3R)-1-(tert-butoxycarbonyl)pyrrolidin-3-yl]methyl}amino)-2-oxoethyl]-N-[1-(2,5-dioxo-2,5-dihydro-1H-pyrrol-1-yl)-2,18-dioxo-6,9,12,15-tetraoxa-3-azaoctadecan-18-yl]-L-cysteine.

LC-MS (Method 12): Rₜ=2.29 min; MS (ESIpos): m/z=1082 [M+H]⁺

S-[2-({(1R)-1-[1-Benzyl-4-(2,5-difluorophenyl)-1H-pyrrol-2-yl]-2,2-dimethylpropyl}{[1-(tert-butoxycarbonyl)pyrrolidin-3-yl]methyl}amino)-2-oxoethyl]-N-[1-(2,5-dioxo-2,5-dihydro-1H-pyrrol-1-yl)-2,18-dioxo-6,9,12,15-tetraoxa-3-azaoctadecan-18-yl]-L-cysteine (17.0 mg, 15.7 μmol) was dissolved in 2.0 ml of trifluoroethanol. Zinc chloride (8.56 mg, 62.8 μmol) was added and the reaction mixture was stirred at 50° C. for 1 hour. Once more, zinc chloride (8.56 mg, 62.8 μmol) was added and the reaction mixture was stirred at 50° C. for 2 hours. Ethylenediamine-N,N,N′,N′-tetraacetic acid (36.7 mg, 126 μmol) was added to the mixture, water (0.1% TFA) was then added and the mixture was subsequently concentrated under reduced pressure. The residue was purified by prep. RP-HPLC (column: Reprosil 250×30; 10μ, flow rate: 50 ml/min, MeCN/water, 0.1% TFA). The solvents were evaporated under reduced pressure and the residue was dried under high vacuum. This gave 3.90 mg (22% of theory) of the title compound.

LC-MS (Method 1): Rₜ=0.89 min; MS (ESIpos): m/z=983 [M+H]⁺

### Intermediate F325

### N-[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]-N²—[(2,5-dioxo-2,5-dihydro-1H-pyrrol-1-yl)acetyl]-D-alpha-glutamine/trifluoroacetic acid (1:1)

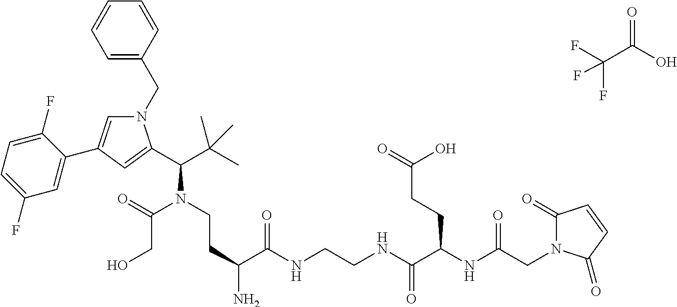 [SMILES: CC(C)(C)[C@H](c1cc(-c2cc(F)ccc2F)cn1Cc1ccccc1)N(CC[C@H](N)C(=O)NCCNC(=O)[C@@H](CCC(=O)O)NC(=O)CN1C(=O)C=CC1=O)C(=O)CO ; O=C(O)C(F)(F)F]

30 mg (0.046 mmol) of intermediate C58 were coupled with 29 mg (0.055 mmol) of trifluoroacetic acid/benzyl N-(2-aminoethyl)-N2-[(benzyloxy)carbonyl]-D-alpha-glutaminate (1:1) in the presence of 1.5 equiv, of HATU and 3 equiv, of N,N-diisopropylethylamine. Purification by preparative HPLC gave 39.5 mg (82% of theory) of the protected intermediate. First, the benzyl ester groups of this intermediate were removed hydrogenolytically. Subsequent coupling with 1-{2-[(2,5-dioxopyrrolidin-1-yl)oxy]-2-oxoethyl}-1H-pyrrole-2,5-dione in DMF in the presence of 3 equiv, of N,N-diisopropylethylamine and removal of the Teoc protective group with zinc chloride in trifluoroethanol as for intermediate F119 then gave, in 2 further steps, the title compound.

LC-MS (Method 12): Rₜ=1.44 min; MS (ESIpos): m/z=822 (M+H)⁺.

### Intermediate F326

### N-[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]-N²-(bromoacetyl)-D-alpha-glutamine/trifluoro acetic acid (1:1)

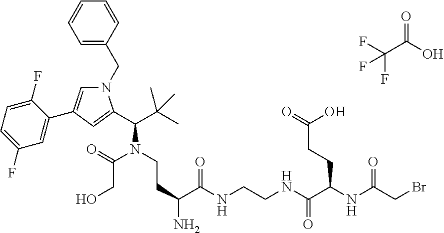 [SMILES: CC(C)(C)[C@H](c1cc(-c2cc(F)ccc2F)cn1Cc1ccccc1)N(CC[C@H](N)C(=O)NCCNC(=O)[C@@H](CCC(=O)O)NC(=O)CBr)C(=O)CO ; O=C(O)C(F)(F)F]

43 mg (0.066 mmol) of intermediate C58 were coupled with 57 mg (0.077 mmol) of intermediate L125 in the presence of 1.5 equiv, of HATU and 4 equiv, of 4-methylmorpholine. Purification by preparative HPLC gave 27 mg (34% of theory) of the protected intermediate. This was then converted into the title compound using zinc chloride in trifluoroethanol as described for intermediate F119.

LC-MS (Method 1): Rₜ=0.83 min; MS (ESIpos): m/z=805 and 807 (M+H)⁺.

### B: Preparation of Antibody Drug Conjugates (ADC)

### B-1. General Process for Generating ITEM-4 and Chimeric and Humanized Variants of ITEM-4

ITEM-4 is an anti-TWEAKR antibody described by Nakayama et al. (Nakayama, et al., 2003, Biochem Biophy Res Comm, 306:819-825). The sequences of the variable regions (VH and VL) of ITEM-4 were disclosed in Zhou et al. (Zhou et al., 2013, J Invest Dermatol. 133(4): 1052-62). Humanized variants of this antibody based on CDR grafting in human framework regions have been described by Zhou et al. (Zhou et al., 2013, J Invest Dermatol. 133(4): 1052-62) and in WO 2009/020933.

Based on the publication of the sequences of the variable regions (VH and VL) of ITEM-4, in Zhou et al. (Zhou et al., 2013, J Invest Dermatol. 133(4): 1052-62) and the publication of the humanized variants of this antibody based on CDR grafting in human framework regions Zhou et al. (Zhou et al., 2013, J Invest Dermatol. 133(4): 1052-62) and WO 2009/020933, the following antibody sequences were obtained: “TPP-7007”, “TPP-7053”, “TPP-7005”, “TPP-7073”, “TPP-7075” and “TPP-7076”.

Linking the variable regions VH and VL of ITEM-4 to the constant regions (CL, CH1, CH2, CH3) of a human IgG1 of the kappa subtype the sequences of the chimeric antibodies “TPP-7006” and “TPP-7074” are obtained.

Modification of a potential deamidation site in L-CDR1 of ITEM-4 gives the antibodies “TPP-7073”, “TPP-7074”, “TPP-7075” and “TPP-7076”.

Further humanized variants of the ITEM-4 antibody can be generated by humanization processes known in the art.

ITEM-4 was obtained commercially (inter alia from eBioscience® as 13-9018 (Ref 7016-9018 M010)

### B-2. General Process for Expressing Anti-TWEAKR Antibodies in Mammalian Cells

The antibodies can be produced in transient cultures of mammalian cells, as described by Tom et al., Chapter 12 in Methods Express: Expression Systems, edited by Micheal R. Dyson and Yves Durocher, Scion Publishing Ltd, 2007 (see AK—Example 1).

B-3. General Process for Purifying Antibodies from Cell Supernatants

The antibodies can be obtained from the cell culture supernatants. To this end, the cell supernatants are clarified by centrifugation of cells. The cell supernatant is then purified by affinity chromatography on a MabSelect Sure (GE Healthcare) chromatography column. To this end, the column is equilibrated in DPBS pH 7.4 (Sigma/Aldrich), the cell supernatant is applied and the column is washed with about 10 column volumes of DPBS pH 7.4+500 mM sodium chloride. The antibodies are eluted in 50 mM sodium acetate pH 3.5+500 mM sodium chloride and then purified further by gel filtration chromatography on a Superdex 200 column (GE Healthcare) in DPBS pH 7.4.

### B-4. General Process for Coupling to Cysteine Side Chains

The following antibodies were used for the coupling reactions:

anti-TWEAKR AK_{1A} (ITEM-4)

anti-TWEAKR AK_{1B} (TPP-7005)

anti-TWEAKR AK_{1C} (TPP-7006)

anti-TWEAKR AK_{1D} (TPP-7007)

Between 2 and 5 equivalents of tris(2-carboxyethyl)phosphine hydrochloride (TCEP), dissolved in PBS buffer, were added to a solution of the appropriate antibody in PBS buffer in the concentration range between 1 mg/ml and 20 mg/ml, preferably in the range of about 5 mg/ml to 15 mg/ml, and the mixture was stirred at RT for 1 h. For this purpose, the solution of the respective antibody used can be employed at the concentrations stated in the working examples, or it may optionally also be diluted with PBS buffer to about half of the stated starting concentrations in order to get into the preferred concentration range. Subsequently, depending on the intended loading, from 2 to 12 equivalents, preferably about 5-10 equivalents of the maleinimide precursor compound or halide precursor compound to be coupled were added as a solution in DMSO. Here, the amount of DMSO should not exceed 10% of the total volume. The reaction was stirred in the case of maleinimide precursors for 60-240 min at RT and in the case of halide precursors between 8 and 24 h at RT and then applied to PBS-equilibrated PD 10 columns (Sephadex® G-25, GE Healthcare) and eluted with PBS buffer. Generally, unless indicated otherwise, 5 mg of the antibody in question in PBS buffer were used for the reduction and the subsequent coupling. Purification on the PD 10 column thus in each case afforded solutions of the respective ADCs in 3.5 ml PBS buffer. The sample was then concentrated by ultracentrifugation and optionally rediluted with PBS buffer. If required, for better removal of low-molecular weight components, concentration by ultrafiltration was repeated after redilution with PBS buffer. For biological tests, if required, the concentrations of the final ADC samples were optionally adjusted to the range of 0.5-15 mg/ml by redilution. The respective protein concentrations, stated in the working examples, of the ADC solutions were determined. Furthermore, antibody loading (drug/mAb ratio) was determined using the methods described under B-7.

Depending on the linker, the ADCs shown in the examples may also be present to a lesser or higher degree in the form of the hydrolysed open-chain succinamides attached to the antibodies. In particular the KSP-I-ADCs attached though the linker substructure

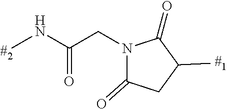

to thiol groups of the antibodies may optionally also be prepared in a targeted manner by rebuffering after the coupling and stirring at pH 8 for about 20-24 h according to Scheme 26 via the ADCs attached via open-chain succinamides.

#1 represents the sulphur bridge to the antibody, and #2 the point of attachment to the modified KSP inhibitor

Such ADCs where the linker is attached to the antibodies through hydrolysed open-chain succinamides may optionally also be prepared in a targeted manner by an exemplary procedure as follows:

### Small Scale Coupling:

From 2 to 5 equivalents of tris(2-carboxyethyl)phosphine hydrochloride (TCEP), dissolved in PBS buffer, were added to a solution of 2-5 mg of the antibody in question in PBS buffer in a concentration range of from 1 mg/ml to 20 mg/ml, preferably in the range from about 5 mg/ml to 15 mg/ml, and the mixture was stirred at RT for 30 min to 1 h. For this purpose, the solution of the respective antibody used can be employed in the concentration given in the working examples or optionally also be diluted with PBS buffer to about half of the stated starting concentration to get to the preferred concentration range. Subsequently, depending on the intended charging, from 2 to 12 equivalents, preferably about 5-10 equivalents, of the maleinimide precursor compound to be coupled were added as a solution in DMSO. Here, the amount of DMSO should not exceed 10% of the total volume. The mixture is stirred at RT for 60-240 min and then diluted with PBS buffer, which had been adjusted to pH 8 beforehand, to a volume of 3-7 ml and stirred under argon at RT overnight. This solution was then passed over a PD 10 column (Sephadex® G-25, GE Healthcare) equilibrated with PBS buffer pH 7.2 and eluted with PBS buffer pH 7.2. The eluate was subsequently concentrated by ultracentrifugation and back-diluted with PBS buffer (pH 7.2).

### Medium Scale Coupling:

Under argon, a solution of 0.344 mg TCEP in 100 μl of PBS buffer was added to 60 mg of the antibody in question in 5 ml of PBS buffer (c˜12 mg/ml). The reaction was stirred at RT for 30 min, and 0.003 mmol of a maleinimide precursor compound dissolved in 600 μl of DMSO was then added. After a further 1.5 h-2 h of stirring at RT, the reaction was diluted with 1075 μl of PBS buffer which had been adjusted to pH 8 beforehand.

This solution was then applied to PD 10 columns (Sephadex® G-25, GE Healthcare) which had been equilibrated with PBS buffer pH 8 and was eluted with PBS buffer pH 8. The eluate was diluted with PBS buffer pH 8 to a total volume of 14 ml. This solution was stirred at RT under argon overnight. If required, the solution was then rebuffered to pH 7.2. The ADC solution was concentrated by ultracentrifugation, rediluted with PBS buffer (pH 7.2) and then optionally concentrated again to a concentration of about 10 mg/ml.

Other potentially hydrolysis-sensitive thianylsuccinimide bridges to the antibody in the working examples contain the following linker substructures, where #1 represents the thioether linkage to the antibody and #2 the point of attachment to the modified KSP inhibitor:

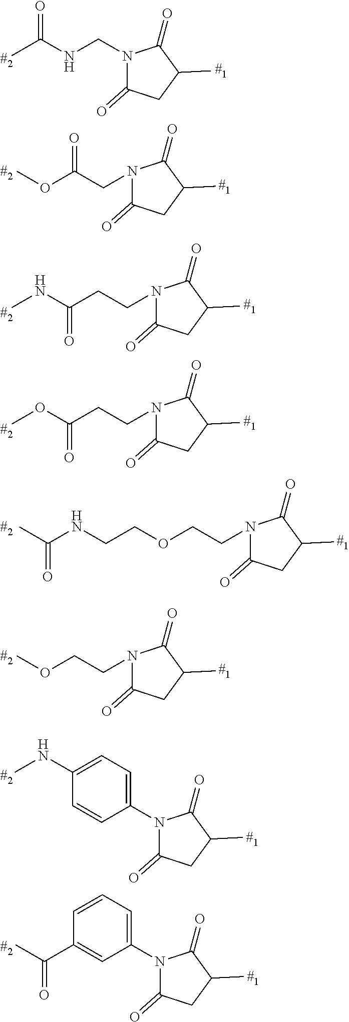

These linker substructures represent the linking unit to the antibody and have (in addition to the linker composition) a significant effect on the structure and the profile of the metabolites formed in the tumour cells.

In the structural formulae shown, AK₁ₐ, AK_{1b}, AK_{1C} and AK_{1D} have the meaning:

AK_{1A}=anti-TWEAKR AK_{1A} (ITEM-4) (partially reduced)-S§¹

AK_{1B}=anti-TWEAKR AK_{1B} (TPP-7005) (partially reduced)-S§¹

AK_{1C}=anti-TWEAKR AK_{1C} (TPP-7006) (partially reduced)-S§¹

AK_{1D}=anti-TWEAKR AK_{1D} (TPP-7007) (partially reduced)-S§¹

where
§¹ represents the linkage to the succinimide group or to any isomeric hydrolysed open-chain succinamides or the alkylene radical resulting therefrom,
and
S represents the sulphur atom of a cysteine residue of the partially reduced antibody.

### B-5. General Process for Coupling to Lysine Side Chains

These couplings are described for example in the Working examples 194k and 294k. Such linkages with the antibody can be used in ADCs with KSP inhibitors, in particular in connection with a 2-8 oligopeptide group SG1 cleavable in vivo, which is linked with R4 via CO.

The following antibodies were used for the coupling reactions:

anti-TWEAKR AK_{1A} (ITEM-4)

anti-TWEAKR AK_{1B} (TPP-7005)

anti-TWEAKR AK_{1C} (TPP-7006)

anti-TWEAKR AK_{1D} (TPP-7007)

From 2 to 8 equivalents of the precursor compound to be coupled were added as a solution in DMSO to a solution of the antibody in question in PBS buffer in a concentration range between 1 mg/ml and 20 mg/ml, preferably about 10 mg/ml, depending on the intended loading. After 30 min to 6 h of stirring at RT, the same amount of precursor compound in DMSO was added again. Here, the amount of DMSO should not exceed 10% of the total volume. After a further 30 min to 6 h of stirring at RT, the reaction was applied to PD 10 columns (Sephadex® G-25, GE Healthcare) equilibrated with PBS and eluted with PBS buffer. Generally, unless indicated otherwise, 5 mg of the antibody in question in PBS buffer were used for the reduction and the subsequent coupling. Purification on the PD 10 column thus in each case afforded solutions of the respective ADCs in 3.5 ml PBS buffer. The sample was then concentrated by ultracentrifugation and optionally rediluted with PBS buffer. If required, for better removal of low-molecular weight components, concentration by ultrafiltration was repeated after redilution with PBS buffer. For biological tests, if required, the concentrations of the final ADC samples were optionally adjusted to the range of 0.5-15 mg/ml by redilution.

The respective protein concentrations, stated in the working examples, of the ADC solutions were determined. Furthermore, antibody loading (drug/mAb ratio) was determined using the methods described under B-7.

In the structural formulae shown, AK₂ₐ, AK_{2b}, AK_{2C} and AK_{2D} have the meaning

AK_{2A}=anti-TWEAKR AK_{1A} (ITEM-4)-NH§²

AK_{2B}=anti-TWEAKR AK_{1B} (TPP-7005)-NH§²

AK_{2C}=anti-TWEAKR AK_{1C} (TPP-7006)-NH§²

AK_{2D}=anti-TWEAKR AK_{1D} (TPP-7007)-NH§²

where
§² represents the linkage to the carbonyl group
and
NH represents the side-chain amino group of a lysine residue of the antibody.
B-6a. General Process for Preparing Closed Succinimide-Cysteine Adducts:

In an exemplary embodiment, 10 μmol of the maleinimide precursor compounds described above were taken up in 3-5 ml of DMF, and 2.1 mg (20 μmol) of L-cysteine were added. The reaction mixture was stirred at RT for 2 h to 24 h, then concentrated under reduced pressure and then purified by preparative HPLC.

B-6aa. General Process for Preparing Isomeric Open Succinamide-Cysteine Adducts:

In an exemplary embodiment, 68 μmol of the maleinimide precursor compounds described above were taken up in 15 ml of DMF, and with 36 mg (136 μmol) of N-{[2-(trimethylsilyl)ethoxy]carbonyl}-L-cysteine were added. The reaction mixture was stirred at RT for 20 h, then concentrated under reduced pressure and then purified by preparative HPLC. The appropriate fractions were combined and the solvents were evaporated under reduced pressure, and the residue was then dissolved in 15 ml of THF/water 1:1. 131 μl of a 2M aqueous lithium hydroxide solution were added and the reaction was stirred at RT for 1 h. The reaction was then neutralized with a 1M hydrochloric acid, the solvent was evaporated under reduced pressure and the residue was purified by preparative HPLC. This gave ˜50% of theory of the regioisomeric protected intermediates as a colourless foam.

In the last step, 0.023 mmol of these regioisomeric hydrolysis products were dissolved in 3 ml of 2,2,2-trifluoroethanol. 12.5 mg (0.092 mmol) of zinc chloride were added, and the reaction was stirred at 50° C. for 4 h. 27 mg (0.092 mmol) of ethylenediamine-N,N,N′,N′-tetraacetic acid were then added, and the solvent was evaporated under reduced pressure. The residue was purified by preparative HPLC. Concentration of the appropriate fractions and lyophilization of the residue from acetonitrile/water gave the hydrolysed open sulphanylsuccinamides as a regioisomer mixture.

### Further Purification and Characterization of the Conjugates According to the Invention

After the reaction, in some instances the reaction mixture was concentrated, for example by ultrafiltration, and then desalted and purified by chromatography, for example using a Sephadex® G-25 column. Elution was carried out, for example, with phosphate-buffered saline (PBS). The solution was then sterile filtered and frozen. Alternatively, the conjugate can be lyophylized.

### B-7. Determination of the Antibody, the Toxophor Loading and the Proportion of Open Cysteine Adducts

For protein identification in addition to molecular weight determination after deglycosylation and/or denaturing, a tryptic digestion was carried out which, after denaturing, reduction and derivatization, confirms the identity of the protein via the tryptic peptides found.

The toxophor loading of the PBS buffer solutions obtained of the conjugates described in the working examples was determined as follows:

Determination of toxophor loading of lysine-linked ADCs was carried out by mass spectrometric determination of the molecular weights of the individual conjugate species. Here, the antibody conjugates were first deglycosylated with PNGaseF, and the sample was acidified and, after HPLC separation/desalting, analysed by mass spectrometry using ESI-MicroTofq (Bruker Daltonik). All spectra over the signal in the TIC (Total Ion Chromatogram) were added and the molecular weight of the different conjugate species was calculated based on MaxEnt deconvolution. The DAR (=drug/antibody ratio) was then calculated after signal integration of the different species.

The toxophor loading of cysteine-linked conjugates was determined by reversed-phase chromatography of the reduced and denatured ADCs. Guanidinium hydrochloride (GuHCl) (28.6 mg) and a solution of DL-dithiothreitol (DTT) (500 mM, 3 μl) were added to the ADC solution (1 mg/ml, 50 μl). The mixture was incubated at 55° C. for one hour and analysed by HPLC.

HPLC analysis was carried out on an Agilent 1260 HPLC system with detection at 220 nm. A Polymer Laboratories PLRP-S polymeric reversed-phase column (catalogue number PL1912-3802) (2.1×150 mm, 8 μm particle size, 1000 Å) was used at a flow rate of 1 ml/min with the following gradient: 0 min, 25% B; 3 min, 25% B; 28 min, 50% B. Mobile phase A consisted of 0.05% trifluoroacetic acid (TFA) in water, mobile phase B of 0.05% trifluoroacetic acid in acetonitrile.

The detected peaks were assigned by retention time comparison with the light chain (L0) and the heavy chain (H0) of the non-conjugated antibody. Peaks detected exclusively in the conjugated sample were assigned to the light chain with one toxophor (L1) and the heavy chains with one, two and three toxophors (H1, H2, H3).

Average loading of the antibody with toxophors was calculated from the peak areas determined by integration as double the sum of HC load and LC load, where LC load is calculated from the sum of the toxophor number-average weighed integration results of all LC peaks divided by the sum of the singly weighed integration results of all LC peaks, and where the HC-load is calculated from the sum of the toxophor number-average weighed integration results of all HC peaks divided by the sum of the singly weighed integration results of all HC peaks. In individual cases, it may not be possible to determine the toxophor load accurately owing to co-elutions of some peaks.

In the cases where light and heavy chains could not be separated sufficiently by HPLC, determination of toxophor loading of cysteine-linked conjugates was carried out by mass spectrometric determination of the molecular weights of the individual conjugate species at light and heavy chain.

Guanidinium hydrochloride (GuHCl) (28.6 mg) and a solution of DL-dithiothreitol (DTT) (500 mM, 3 μl) were added to the ADC solution (1 mg/ml, 50 μl). The mixture was incubated for one hour at 55° C. and analysed by mass spectrometry after online desalting using ESI-MicroTofq (Bruker Daltonik).

For the DAR determination, all spectra were added over the signal in the TIC (Total Ion Chromatogram), and the molecular weight of the different conjugate species at light and heavy chain was calculated based on MaxEnt deconvolution. Average loading of the antibody with toxophors was calculated from the peak areas determined by integration as double the sum of HC load and LC load, where LC load is calculated from the sum of the toxophor number-average weighed integration results of all LC peaks divided by the sum of the singly weighed integration results of all LC peaks, and where the HC-load is calculated from the sum of the toxophor number-average weighed integration results of all HC peaks divided by the sum of the singly weighed integration results of all HC peaks.

To determine the proportion of the open cysteine adduct, the molecular weight area ratio of closed to open cysteine adduct (molecular weight delta 18 Dalton) of all singly conjugated light and heavy chain variants was determined. The mean of all variants yielded the proportion of the open cysteine adduct.

### B-8. Checking the Antigen-Binding of the ADC

The capability of the binder of binding to the target molecule was checked after coupling had taken place. The person skilled in the art is familiar with multifarious methods which can be used for this purpose; for example, the affinity of the conjugate can be checked using ELISA technology or surface plasmon resonance analysis (BIAcore™ measurement). The conjugate concentration can be measured by the person skilled in the art using customary methods, for example for antibody conjugates by protein determination, (see also Doronina et al.; Nature Biotechnol. 2003; 21:778-784 and Poison et al., Blood 2007; 1102:616-623).

### METABOLITE EMBODIMENTS

### Example M1

### S-[1-(2-{[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)-2,5-dioxopyrrolidin-3-yl]-L-cysteine/trifluoroacetic acid (1:1)

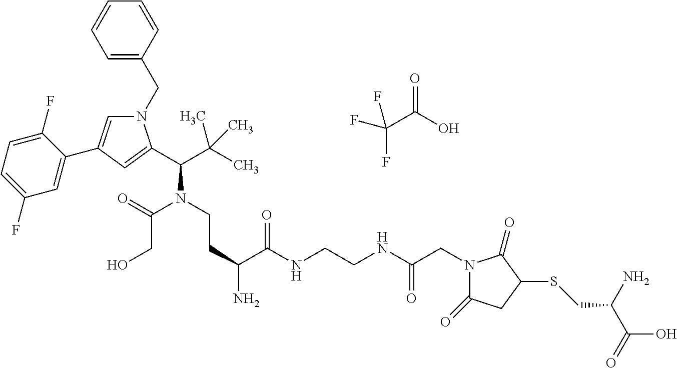 [SMILES: CC(C)(C)[C@H](c1cc(-c2cc(F)ccc2F)cn1Cc1ccccc1)N(CC[C@H](N)C(=O)NCCNC(=O)CN1C(=O)CC(SC[C@H](N)C(=O)O)C1=O)C(=O)CO ; O=C(O)C(F)(F)F]

1.8 mg (2 μmol) of Intermediate F104 were taken up in 1 ml of DMF, and 2.7 mg (22 μmol) of L-cysteine were added. The reaction mixture was stirred at RT for 20 h, then concentrated under reduced pressure and then purified by preparative HPLC. 0.6 mg (26% of theory) of the title compound remained as a colourless foam.

LC-MS (Method 1): Rₜ=0.80 min; MS (EIpos): m/z=814 [M+H]⁺.

### Example M2

### 4-[(2-{[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)amino]-3-{[(2R)-2-amino-2-carboxyethyl]sulphanyl}-4-oxobutanoic acid/trifluoroacetic acid (1:1)

### and

### 4-[(2-{[2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)amino]-2-{[(2R)-2-amino-2-carboxyethyl]sulphanyl}-4-oxobutanoic acid/trifluoroacetic acid (1:1)

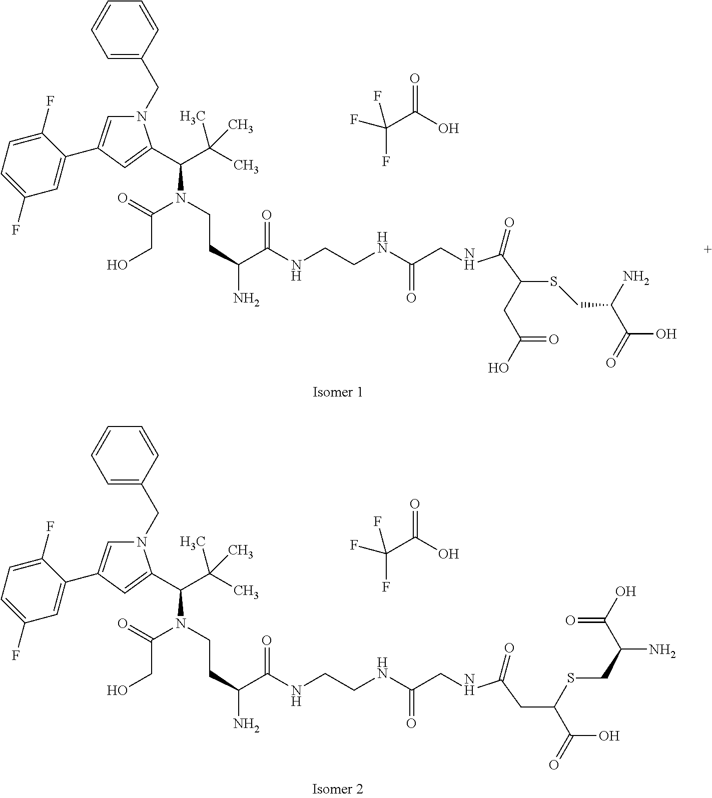 [SMILES: CC(C)(C)[C@H](c1cc(-c2cc(F)ccc2F)cn1Cc1ccccc1)N(CC[C@H](N)C(=O)NCCNC(=O)CNC(=O)C(CC(=O)O)SC[C@H](N)C(=O)O)C(=O)CO ; O=C(O)C(F)(F)F ; CC(C)(C)[C@H](c1cc(-c2cc(F)ccc2F)cn1Cc1ccccc1)N(CC[C@H](N)C(=O)NCCNC(=O)CNC(=O)CC(SC[C@H](N)C(=O)O)C(=O)O)C(=O)CO ; O=C(O)C(F)(F)F]

LC-MS (Method 1): Rₜ=0.80 min; MS (EIpos): m/z=814 [M+H]⁺.

First, L-cysteine was converted with 1-({[2-(trimethylsilyl)ethoxy]carbonyl}oxy)pyrrolidine-2,5-dione in DMF in the presence of N,N-diisopropylethylamine into N-{[2-(trimethylsilyl)ethoxy]carbonyl}-L-cysteine.

406 mg (1.53 mmol) of N-{[2-(trimethylsilyl)ethoxy]carbonyl}-L-cysteine were dissolved in 10 ml of DMF, 157.5 mg (1.606 mmol) of maleic anhydride were added and the reaction was stirred at RT for 1 hour. 7.5 mg (0.01 mmol) of intermediate C66 were added to 130 μl of this solution, and the reaction was stirred at RT for 5 min. The mixture was then concentrated under reduced pressure, and the residue was purified by preparative HPLC. The solvent was evaporated under reduced pressure and the residue was dried under high vacuum. This gave 10 mg (89%) of the protected intermediate; it was not possible to separate the regioisomers neither by HPLC nor by LC-MS.

LC-MS (Method 1): Rₜ=1.38 min; MS (EIpos): m/z=1120 [M+H]⁺.

In the last step, the 10 mg of this intermediate were dissolved in 2 ml of 2,2,2-trifluoroethanol. 12 mg (0.088 mmol) of zinc chloride were added, and the reaction was stirred at 50° C. for 30 min. 26 mg (0.088 mmol) of ethylenediamine-N,N,N′,N′-tetraacetic acid were then added, and the solvent was evaporated under reduced pressure. The residue was purified by preparative HPLC. Concentration of the appropriate fractions and lyophilization of the residue from acetonitrile/water gave 8.3 mg (99% of theory) of the title compound as a regioisomer mixture in a ratio of 87:13.

LC-MS (Method 5): Rₜ=2.3 min and 2.43 min; MS (ESIpos): m/z=832 (M+H)⁺.

¹H-NMR main regioisomer: (500 MHz, DMSO-d₆): δ=8.7 (m, 1H), 8.5 (m, 2H), 8.1 (m, 1H), 7.6 (m, 1H), 7.5 (s, 1H) 7.4-7.15 (m, 6H), 6.9-7.0 (m, 1H), 6.85 (s, 1H), 5.61 (s, 1H), 4.9 and 5.2 (2d, 2H), 4.26 and 4.06 (2d, 2H), 3.5-3.8 (m, 5H), 3.0-3.4 (m, 5H), 2.75-3.0 (m, 3H), 2.58 and 2.57 (dd, 1H), 0.77 and 1.5 (2m, 2H), 0.81 (s, 9H).

Alternatively, the regioisomeric title compounds were prepared as follows:

To this end, first L-cysteine was converted with 1-({[2-(trimethylsilyl)ethoxy]carbonyl}oxy)pyrrolidine-2,5-dione in DMF in the presence of N,N-diisopropylethylamine into N-{[2-(trimethylsilyl)ethoxy]carbonyl}-L-cysteine.

55 mg (0.068 mmol) of Intermediate F104 and 36 mg (0.136 mmol) of N-{[2-(trimethylsilyl) ethoxy]carbonyl}-L-cysteine were dissolved in 15 ml of DMF, and the mixture was stirred at RT for 20 h. The mixture was then concentrated and the residue was purified by preparative HPLC. The appropriate fractions were combined and the solvents were evaporated under reduced pressure, and the residue was then dissolved in 15 ml of THF/water 1:1. 131 μl of a 2M aqueous lithium hydroxide solution were added and the reaction was stirred at RT for 1 h. The reaction was then neutralized with a 1M hydrochloric acid, the solvent was evaporated under reduced pressure and the residue was purified by preparative HPLC. This gave 37 mg (50% of theory) of the regioisomeric protected intermediates as a colourless foam.

LC-MS (Method 5): Rₜ=3.33 min and 3.36 min; MS (ESIpos): m/z=976 (M+H)⁺.

In the last step, 25 mg (0.023 mmol) of this intermediate were dissolved in 3 ml of 2,2,2-trifluoroethanol. 12.5 mg (0.092 mmol) of zinc chloride were added, and the reaction was stirred at 50° C. for 4 h. 27 mg (0.092 mmol) of ethylenediamine-N,N,N′,N′-tetraacetic acid were then added, and the solvent was evaporated under reduced pressure. The residue was purified by preparative HPLC. Concentration of the appropriate fractions and lyophilization of the residue from acetonitrile/water gave 18.5 mg (85% of theory) of the title compound as a regioisomer mixture in a ratio of 21:79.

LC-MS (Method 5): Rₜ=2.37 min and 3.44 min; MS (ESIpos): m/z=832 (M+H)⁺.

### Example M3

### 4-[(2-{[(2R)-2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)-2-carboxyethyl]amino}-2-oxoethyl)amino]-3-{[(2R)-2-amino-2-carboxyethyl]sulphanyl}-4-oxobutanoic acid/trifluoroacetic acid (1:1)

### and

### 4-[(2-{[(2R)-2-({(2S)-2-amino-4-[{(1R)-1-[1-benzyl-4-(2,5-difluorophenyl)-1H-pyrrol-2-yl]-2,2-dimethylpropyl}(glycoloyl)amino]butanoyl}amino)-2-carboxyethyl]amino}-2-oxoethyl)amino]-2-{[(2R)-2-amino-2-carboxyethyl]sulphanyl}-4-oxobutanoic acid/trifluoroacetic acid (1:1)

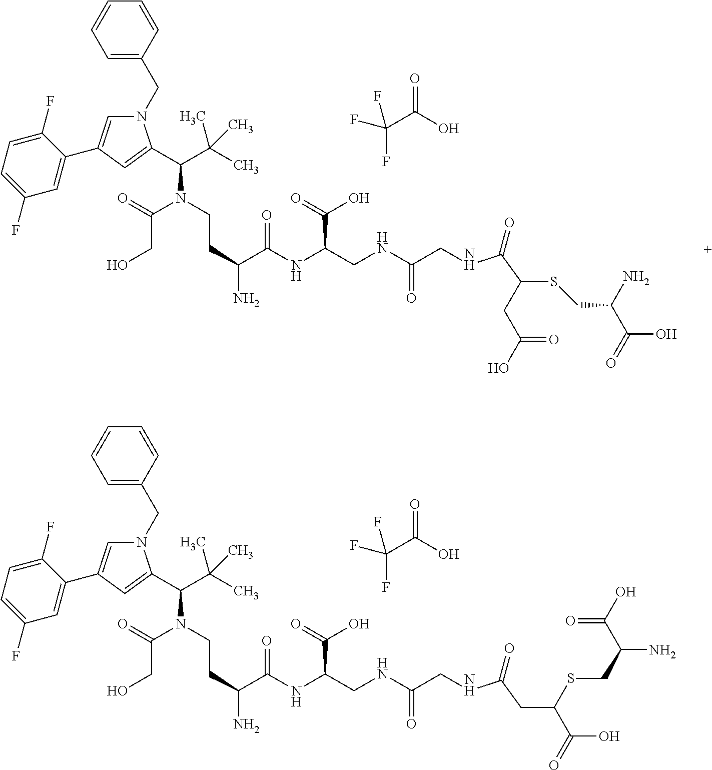 [SMILES: CC(C)(C)[C@H](c1cc(-c2cc(F)ccc2F)cn1Cc1ccccc1)N(CC[C@H](N)C(=O)N[C@H](CNC(=O)CNC(=O)C(CC(=O)O)SC[C@H](N)C(=O)O)C(=O)O)C(=O)CO ; O=C(O)C(F)(F)F ; CC(C)(C)[C@H](c1cc(-c2cc(F)ccc2F)cn1Cc1ccccc1)N(CC[C@H](N)C(=O)N[C@H](CNC(=O)CNC(=O)CC(SC[C@H](N)C(=O)O)C(=O)O)C(=O)O)C(=O)CO ; O=C(O)C(F)(F)F]

First, L-cysteine was converted with 1-({[2-(trimethylsilyl)ethoxy]carbonyl}oxy)pyrrolidine-2,5-dione in DMF in the presence of N,N-diisopropylethylamine into N-{[2-(trimethylsilyl)ethoxy]carbonyl}-L-cysteine.

11 mg (0.013 mmol) of Intermediate F193 and 8 mg (0.016 mmol) of N-{[2-(trimethylsilyl) ethoxy]carbonyl}-L-cysteine were dissolved in 3 ml of DMF, and the mixture was stirred at RT for 20 h. The mixture was then concentrated and the residue was purified by preparative HPLC.

The appropriate fractions were combined and the solvents were evaporated under reduced pressure, and the residue was then dissolved in 2 ml of THF/water 1:1. 19 μl of a 2M aqueous lithium hydroxide solution were added and the reaction was stirred at RT for 1 h. Another 19 μl of the 2M aqueous lithium hydroxide solution were then added and the reaction was stirred at RT overnight. The mixture was then neutralized with a 1M hydrochloric acid, the solvent was evaporated under reduced pressure and the residue was purified by preparative HPLC. This gave 4.1 mg (38% of theory) of the regioisomeric protected intermediates as a colourless foam.

LC-MS (Method 1): Rₜ=1.03 min (breit); MS (ESIpos): m/z=1020 (M+H)⁺.

In the last step, 4.1 mg (0.004 mmol) of this intermediate were dissolved in 3 ml of 2,2,2-trifluoroethanol. 3 mg (0.022 mmol) of zinc chloride were added, and the reaction was stirred at 50° C. for 1 h. 6 mg (0.022 mmol) of ethylenediamine-N,N,N′,N′-tetraacetic acid and 2 ml of a 0.1% strength aqueous trifluoroacetic acid were then added, and the solvent was evaporated under reduced pressure. The residue was purified by preparative HPLC. Concentration of the appropriate fractions and lyophilization of the residue from acetonitrile/water gave 5 mg (quant.) of the title compound as a regioisomer mixture in a ratio of 20:80.

LC-MS (Method 1): Rₜ=0.78 min (breit); MS (ESIpos): m/z=876 (M+H)⁺.

LC-MS (Method 5): Rₜ=2.36 min and 2.39 min; MS (ESIpos): m/z=876 (M+H)⁺.

### Example M4

### S-(1-{2-[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino) ethoxy]ethyl}-2,5-dioxopyrrolidin-3-yl)-L-cysteine/trifluoroacetic acid (1:1)

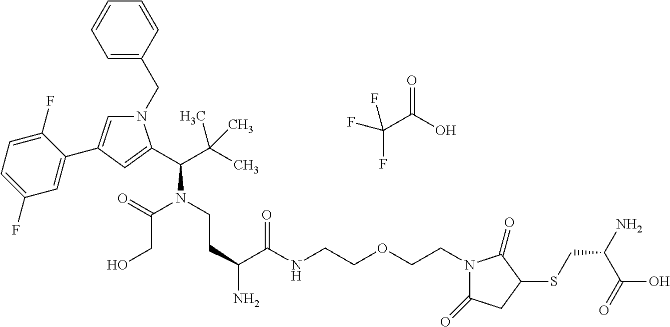 [SMILES: CC(C)(C)[C@H](c1cc(-c2cc(F)ccc2F)cn1Cc1ccccc1)N(CC[C@H](N)C(=O)NCCOCCN1C(=O)CC(SC[C@H](N)C(=O)O)C1=O)C(=O)CO ; O=C(O)C(F)(F)F]

3 mg (4 μmol) of Intermediate F248 were taken up in 2 ml of DMF, and 0.9 mg (8 μmol) of L-cysteine were added. The reaction mixture was stirred at RT for 18 h and then concentrated under reduced pressure. The residue was purified by preparative HPLC. The appropriate fractions were concentrated, giving, after lyophilization of the residue from acetonitrile/water, 1.1 mg (32% of theory) of the title compound as a white solid.

LC-MS (Method 1): Rₜ=0.78 min; MS (EIpos): m/z=801 [M+H]⁺.

### Example M5

### (3R,7S)-7-Amino-17-{[(2R)-2-amino-2-carboxyethyl]sulphanyl}-3-[1-benzyl-4-(2,5-difluorophenyl)-1H-pyrrol-2-yl]-4-glycoloyl-2,2-dimethyl-8,16-dioxo-12-oxa-4,9,15-triazanonadecan-19-oic acid/trifluoroacetic acid (1:1)

### and

### (3R,7S)-7-amino-18-{[(2R)-2-amino-2-carboxyethyl]sulphanyl}-3-[1-benzyl-4-(2,5-difluorophenyl)-1H-pyrrol-2-yl]-4-glycoloyl-2,2-dimethyl-8,16-dioxo-12-oxa-4,9,15-triazanonadecan-19-oic acid/trifluoroacetic acid (1:1)

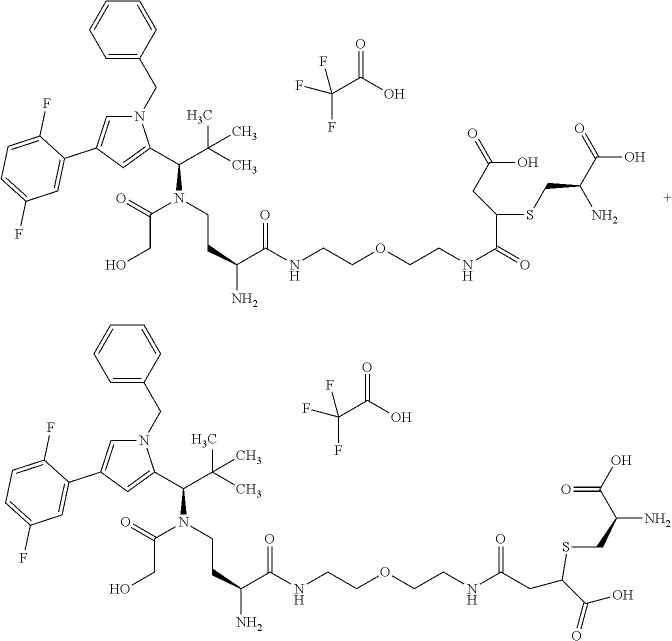 [SMILES: CC(C)(C)[C@H](c1cc(-c2cc(F)ccc2F)cn1Cc1ccccc1)N(CC[C@H](N)C(=O)NCCOCCNC(=O)C(CC(=O)O)SC[C@H](N)C(=O)O)C(=O)CO ; O=C(O)C(F)(F)F ; CC(C)(C)[C@H](c1cc(-c2cc(F)ccc2F)cn1Cc1ccccc1)N(CC[C@H](N)C(=O)NCCOCCNC(=O)CC(SC[C@H](N)C(=O)O)C(=O)O)C(=O)CO ; O=C(O)C(F)(F)F]

8 mg (0.010 mmol) of the protected intermediate of Intermediate F248 and 5.1 mg (0.02 mmol) of N-{[2-(trimethylsilyl) ethoxy]carbonyl}-L-cysteine were dissolved in 3 ml of DMF, and the mixture was stirred at RT for 18 h and then treated in an ultrasonic bath for 2 h. The mixture was then concentrated and the residue was purified by preparative HPLC. The appropriate fractions were combined and the solvents were evaporated under reduced pressure, and the residue was then dissolved in 2 ml of THF/water 1:1. 15 μl of a 2M aqueous lithium hydroxide solution were added and the reaction was stirred at RT for 15 min. The reaction was then adjusted to a pH of 3 with a 1M hydrochloric acid, diluted with 20 ml of sodium chloride solution and extracted twice with 20 ml of ethyl acetate. The organic phase was dried over magnesium sulphate and concentrated, and the residue was lyophilized from acetonitrile/water. This gave 8.4 mg (78% of theory over 2 steps) of the regioisomeric protected intermediates as a colourless foam.

LC-MS (Method 1): Rₜ=1.44 min and 3.43 min; MS (ESIpos): m/z=1107 (M+H)⁺.

In the last step, 8 mg (0.007 mmol) of this intermediate were dissolved in 5 ml of 2,2,2-trifluoroethanol. 9.8 mg (0.072 mmol) of zinc chloride were added, and the reaction was stirred at 50° C. for 1.5 h. Ethylenediamine-N,N,N′,N′-tetraacetic acid were then added, and the solvent was evaporated under reduced pressure. The residue was purified by preparative HPLC. Concentration of the appropriate fractions and lyophilization of the residue from acetonitrile/water gave 4 mg (59% of theory) of the title compound as a regioisomer mixture in a ratio of 31:67.

LC-MS (Method 1): Rₜ=0.79 min and 0.81 min; MS (ESIpos): m/z=819 (M+H)⁺.

### Example M6

### 2-{[(2R)-2-Amino-2-carboxyethyl]sulphanyl}-4-({(14R)-13-(3-aminopropyl)-14-[1-benzyl-4-(2,5-difluorophenyl)-1H-pyrrol-2-yl]-15,15-dimethyl-2,7,12-trioxo-10-thia-3,6,13-triaz ahexadec-1-yl}amino)-4-oxobutanoic acid/trifluoroacetic acid (1:2) and

### 3-{[(2R)-2-amino-2-carboxyethyl]sulphanyl}-4-({(14R)-13-(3-aminopropyl)-14-[1-benzyl-4-(2,5-difluorophenyl)-1H-pyrrol-2-yl]-15,15-dimethyl-2,7,12-trioxo-10-thia-3,6,13-triaz ahexadec-1-yl}amino)-4-oxobutanoic acid/trifluoroacetic acid (1:2)

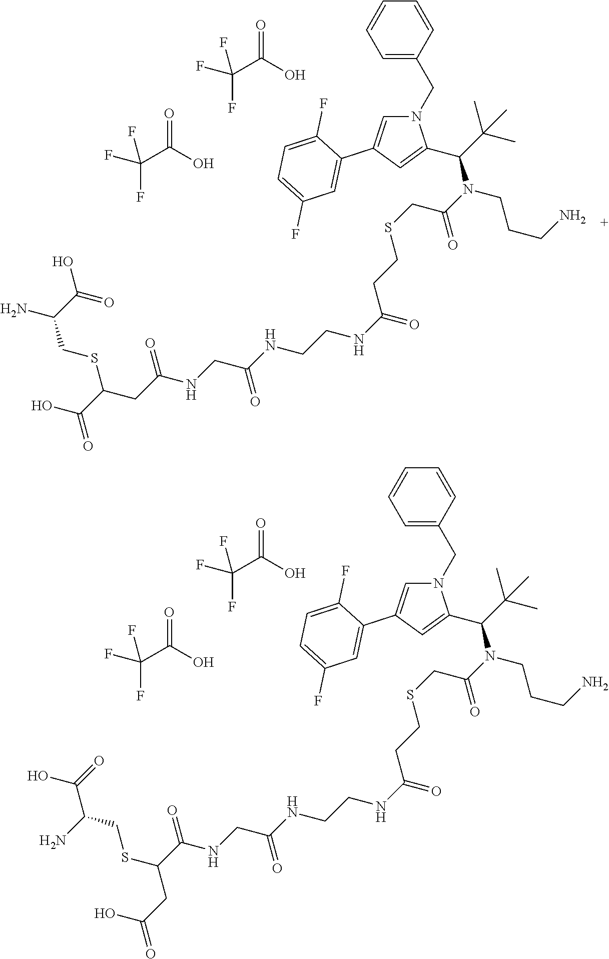

18 mg (0.021 mmol) of Intermediate F213 and 11.2 mg (0.04 mmol) of N-{[2-(trimethylsilyl) ethoxy]carbonyl}-L-cysteine were dissolved in 2 ml of DMF, and the mixture was stirred at RT for 18 h. The reaction mixture was concentrated under reduced pressure. The residue (21.2 mg) was dissolved in 3 ml of THF/water 1:1. 0.04 ml of a 2M aqueous lithium hydroxide solution were added and the reaction was stirred at RT for 3 hours. 0.02 ml of a 2M aqueous lithium hydroxide solution were added and the reaction was stirred at RT for 1 hour. The reaction was then adjusted to a pH of 7 using 7.2 mg (0.12 mmol) of acetic acid. The reaction mixture was purified directly by preparative RP-HPLC (column: Reprosil 125×30; 10μ, flow rate: 50 ml/min, MeCN/water; 0.1% TFA). The solvents were evaporated under reduced pressure and the residue was dried under high vacuum. This gave 13 mg (57% over 2 steps) of the regioisomeric protected intermediates.

LC-MS (Method 1): Rₜ=1.03 min; MS (ESIpos): m/z=1020 (M+H)⁺.

In the last step, 13 mg (0.01 mmol) of this intermediate were dissolved in 2 ml of 2,2,2-trifluoroethanol. 6.2 mg (0.05 mmol) of zinc chloride were added, and the reaction was stirred at 50° C. for 7 h. 13.3 mg (0.05 mmol) of ethylenediamine-N,N,N′,N′-tetraacetic acid were then added, and the product was purified by preparative HPLC. Concentration of the appropriate fractions and lyophilization of the residue from acetonitrile/water gave 10.3 mg (81.4%) of the title compound as a regioisomer mixture.

LC-MS (Method 1): Rₜ=1.03 min; MS (ESIpos): m/z=875 (M+H)⁺.

### Example M7

### S-(2-{[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)-L-cysteine/trifluoroacetic acid (1:1)

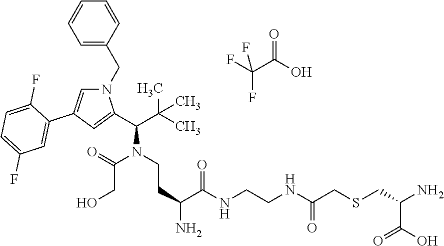 [SMILES: CC(C)(C)[C@H](c1cc(-c2cc(F)ccc2F)cn1Cc1ccccc1)N(CC[C@H](N)C(=O)NCCNC(=O)CSC[C@H](N)C(=O)O)C(=O)CO ; O=C(O)C(F)(F)F]

6 mg (8 μmol) of Intermediate F119 were taken up in 3 ml of DMF, and 1.8 mg (15 μmol) of L-cysteine were added. The reaction mixture was stirred at RT for 6 h and then allowed to stand at RT for 3 days. The reaction was then concentrated under reduced pressure, and the product was purified by preparative HPLC.

LC-MS (Method 1): Rₜ=0.81 min; MS (ESIpos): m/z=717 (M+H)⁺.

### Example M8

### (3R)-6-{(11S,15R)-11-Amino-15-[1-benzyl-4-(2,5-difluorophenyl)-1H-pyrrol-2-yl]-14-glycoloyl-16,16-dimethyl-2,5,10-trioxo-3,6,9,14-tetraazaheptadec-1-yl}-5-oxothiomorpholine-3-carboxylic acid/trifluoroacetic acid (1:1)

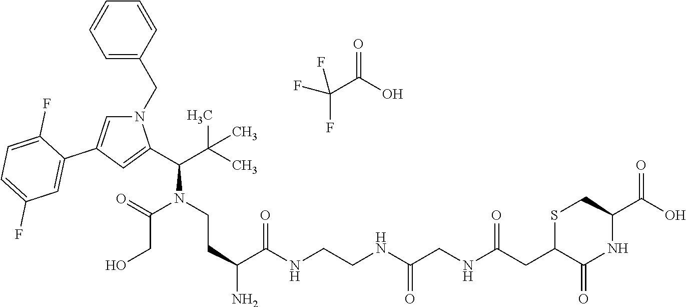

4 mg (0.004 mmol) of the compound from Example 135 were dissolved in 4 ml of THF/water, and 48 μl of a 2-molar aqueous lithium hydroxide solution were added. The reaction was stirred at RT for 1 h and then concentrated and purified by preparative HPLC. Combination, concentration and lyophilization of the appropriate fractions from acetonitrile/water gave 2.4 mg (60% of theory) of the title compound.

LC-MS (Method 1): Rₜ=0.86 min; MS (EIpos): m/z=814 [M+H]⁺.

### Example M9

### N-(3-Aminopropyl)-N-{(1R)-1-[1-benzyl-4-(2,5-difluorophenyl)-1H-pyrrol-2-yl]-2,2-dimethylpropyl}-2-hydroxyacetamide

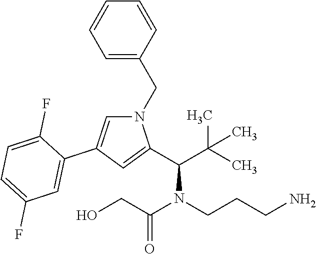 [SMILES: CC(C)(C)[C@H](c1cc(-c2cc(F)ccc2F)cn1Cc1ccccc1)N(CCCN)C(=O)CO]

150.0 mg (0.42 mmol) of (1R)-1-[1-benzyl-4-(2,5-difluorophenyl)-1H-pyrrol-2-yl]-2,2-dimethylpropan-1-amine (Intermediate C52) were initially charged in 2.0 ml of dichloromethane, and 29.2 mg (0.49 mmol) of HOAc and 125.6 mg (0.59 mmol) of sodium triacetoxyborohydride were added and the mixture was stirred at RT for 5 min. 98.9 mg (0.49 mmol) of 3-(1,3-dioxo-1,3-dihydro-2H-isoindol-2-yl)propanal were added. The reaction mixture was stirred at RT overnight. The reaction mixture was diluted with ethyl acetate and the organic phase was washed twice with saturated sodium carbonate solution and once with saturated NaCl solution. After drying over magnesium sulphate, the solvent was evaporated under reduced pressure and the residue was purified on silica gel (mobile phase: dichloromethane/methanol 100:1). The solvents were evaporated under reduced pressure and the residue was dried under high vacuum. This gave 188.6 mg (74%) of the compound 2-[3-({(1R)-1-[1-benzyl-4-(2,5-difluorophenyl)-1H-pyrrol-2-yl]-2,2-dimethylpropyl}amino)propyl]-1H-isoindole-1,3(2H)-dione.

LC-MS (Method 1): Rₜ=1.00 min; MS (ESIpos): m/z=541 [M+H]⁺.

171.2 mg (0.32 mmol) of 2-[3-({(1R)-1-[1-benzyl-4-(2,5-difluorophenyl)-1H-pyrrol-2-yl]-2,2-dimethylpropyl}aminopropyl]-1H-isoindole-1,3(2H)-dione were initially charged in 5.0 ml of dichloromethane, and 73.6 mg (0.73 mmol) of triethylamine were added. At 0° C., 94.9 mg (0.70 mmol) of acetoxyacetyl chloride were added, and the reaction mixture was stirred at RT overnight. The reaction mixture was diluted with ethyl acetate and the organic phase was washed twice with saturated sodium bicarbonate solution and once with sat. NaCl solution. After drying over magnesium sulphate, the solvent was evaporated under reduced pressure and the residue was purified using Biotage Isolera (silica gel, column 10 g SNAP, flow rate 12 ml/min, ethyl acetate/cyclohexane 1:3). The solvents were evaporated under reduced pressure and the residue was dried under high vacuum. This gave 159.0 mg (77%) of the compound 2-({(1R)-1-[1-benzyl-4-(2,5-difluorophenyl)-1H-pyrrol-2-yl]-2,2-dimethylpropyl}[3-(1,3-dioxo-1,3-dihydro-2H-isoindol-2-yl)propyl]amino)-2-oxoethyl acetate.

LC-MS (Method 1): Rₜ=1.35 min; MS (ESIpos): m/z=642 [M+H]⁺.

147.2 mg (0.23 mmol) of 2-({(1R)-1-[1-benzyl-4-(2,5-difluorophenyl)-1H-pyrrol-2-yl]-2,2-dimethylpropyl}[3-(1,3-dioxo-1,3-dihydro-2H-isoindol-2-yl)propyl]amino)-2-oxoethyl acetate were initially charged in 4.0 ml of ethanol, and 356.2 mg (4.59 mmol) of methanamine (40% in water) were added. The reaction mixture was stirred at 50° C. overnight. The solvent was evaporated under reduced pressure and the residue co-distilled three times with toluene. The residue was purified on silica gel (mobile phase: dichloromethane/methanol=10:1). The solvents were evaporated under reduced pressure and the residue was dried under high vacuum. This gave 67.4 mg (63%) of the title compound.

LC-MS (Method 1): Rₜ=0.91 min; MS (ESIpos): m/z=470 [M+H]⁺.

### Example M10

### (2R,28R)-28-Amino-2-[({2-[(3-aminopropyl){(1R)-1-[1-benzyl-4-(2,5-difluorophenyl)-1H-pyrrol-2-yl]-2,2-dimethylpropyl}amino]-2-oxoethyl}sulphanyl)methyl]-25-(carboxymethyl)-4,20,24-trioxo-7,10,13,16-tetraoxa-26-thia-3,19,23-triazanonacosan-1,29-dioic acid/trifluoroacetic acid (1:2) and

### (1R,28R,34R)-1-amino-33-(3-aminopropyl)-34-[1-benzyl-4-(2,5-difluorophenyl)-1H-pyrrol-2-yl]-35,35-dimethyl-6,10,26,32-tetraoxo-14,17,20,23-tetraoxa-3,30-dithia-7,11,27,33-tetraazahexatriacontane-1,4,28-tricarboxylic acid/trifluoroacetic acid (1:2)

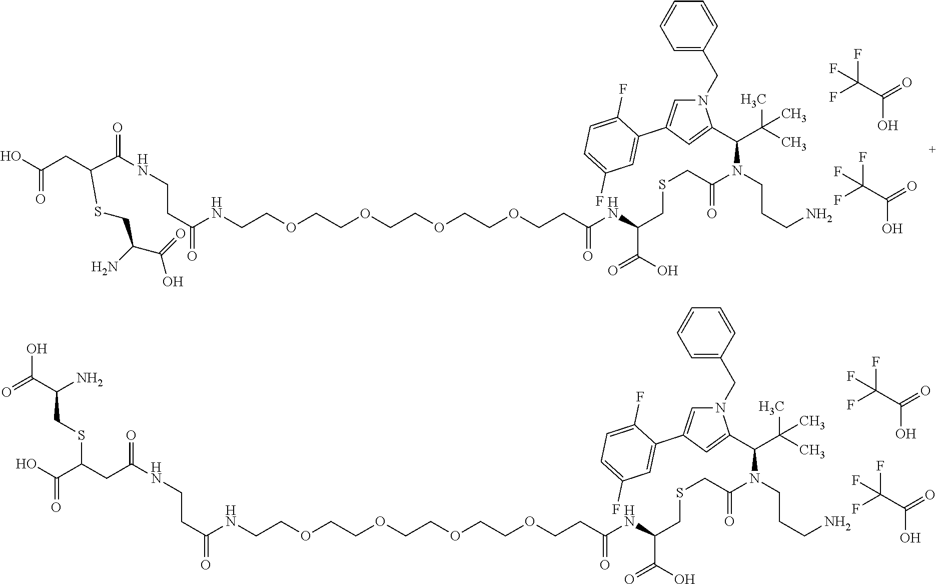

20 mg (0.018 mmol) of R-{2-[(3-aminopropyl) {(1R)-1-[1-benzyl-4-(2,5-difluorophenyl)-1H-pyrrol-2-yl]-2,2-dimethylpropyl}amino]-2-oxoethyl}-N-[19-(2,5-dioxo-2,5-dihydro-1H-pyrrol-1-yl)-17-oxo-4,7,10,13-tetraoxa-16-azanonadecan-1-oyl]-L-cysteine/trifluoroacetic acid (1:1) (Intermediate F209) and 9.78 mg (0.036 mmol) of N-{[2-(trimethylsilyl) ethoxy]carbonyl}-L-cysteine were dissolved in 2 ml of DMF, and the mixture was stirred at RT for 18 h. The reaction mixture was concentrated under reduced pressure. The residue (47.7 mg) was dissolved in 3 ml of THF/water 1:1. 0.08 ml of a 2M aqueous lithium hydroxide solution were added and the reaction was stirred at RT for 1 hour. The reaction was then adjusted to a pH of 7 using 9.26 mg (0.15 mmol) of acetic acid. The reaction mixture was purified directly by preparative RP-HPLC (column: Reprosil 125×30; 10μ, flow rate: 50 ml/min, MeCN/water; 0.1% TFA). The solvents were evaporated under reduced pressure and the residue was dried under high vacuum. This gave 15.3 mg (29% over 2 steps) of the regioisomeric protected intermediates.

LC-MS (Method 6): Rₜ=12.26 min and 12.30 min; MS (ESIpos): m/z=1254 (M+H)⁺.

In the last step, 15.3 mg (0.01 mmol) of this intermediate were dissolved in 2 ml of 2,2,2-trifluoroethanol. 6.1 mg (0.05 mmol) of zinc chloride were added, and the reaction was stirred at 50° C. for 2 h. 13.1 mg (0.05 mmol) of ethylenediamine-N,N,N′,N′-tetraacetic acid were then added, and the product was purified by preparative HPLC. Concentration of the appropriate fractions and lyophilization of the residue from acetonitrile/water gave 11.9 mg (79.5%) of the title compound as a regioisomer mixture.

LC-MS (Method 1): Rₜ=0.85 min; MS (ESIpos): m/z=1110 (M+H)⁺.

### Example M11

### S-{2-[(3-Aminopropyl){(1R)-1-[1-benzyl-4-(2,5-difluorophenyl)-1H-pyrrol-2-yl]-2,2-dimethylpropyl}amino]-2-oxoethyl}-L-cysteine/trifluoroacetic acid (1:2)

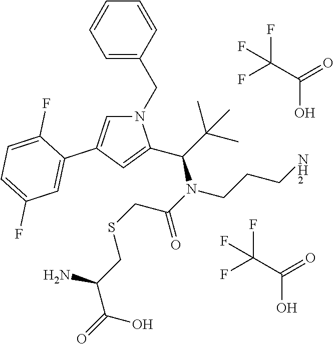

15.0 mg (0.018 mmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-L-cysteine/trifluoroacetic acid (1:1) (Intermediate C71) were dissolved in 1.0 ml of trifluoroethanol, and 7.4 mg (0.054 mmol) of zinc dichloride were added. The reaction mixture was stirred at 50° C. overnight. 15.8 mg (0.054 mmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 11.1 mg (77%) of the title compound.

LC-MS (Method 1): Rₜ=0.83 min; MS (ESIpos): m/z=573 (M+H)⁺.

### Example M12

### 4-{[(1R)-2-({2-[(3-Aminopropyl){(1R)-1-[1-benzyl-4-(2,5-difluorophenyl)-1H-pyrrol-2-yl]-2,2-dimethylpropyl}amino]-2-oxoethyl}sulphanyl)-1-carboxyethyl]amino}-4-oxobutanoic acid/trifluoroacetic acid (1:1)

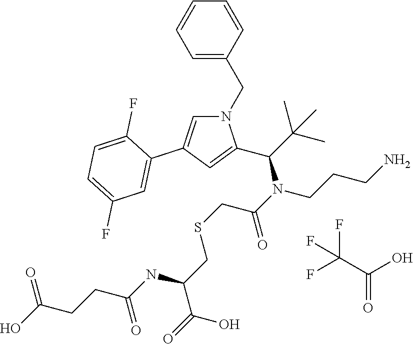

12.2 mg (0.014 mmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-(4-tert-butoxy-4-oxobutanoyl)-L-cysteine (Intermediate C77) were dissolved in 2.0 ml of trifluoroethanol, and 11.4 mg (0.084 mmol) of zinc dichloride were added. The reaction mixture was stirred at 50° C. for 3 h. 24.5 mg (0.084 mmol) of ethylenediamine-N,N,N′,N′-tetraacetic acid were added, the reaction mixture was stirred for 10 min and water (0.1% TFA) was then added. Purification was carried out directly by preparative RP-HPLC (column: Reprosil 125×30; 10μ, flow rate: 50 ml/min, MeCN/water, 0.1% TFA). The solvents were evaporated under reduced pressure and the residue was dried under high vacuum. This gave 4.6 mg (42%) of the title compound.

LC-MS (Method 1): Rₜ=0.88 min; MS (ESIpos): m/z=673 (M+H)⁺.

### Example M13

### 4-[(2-{[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)amino]-2-{[(2R)-2-amino-2-carboxyethyl]sulphanyl}-4-oxobutanoic acid/trifluoroacetic acid (1:1)

### Regioisomer 1, Epimer 1 (2R) or (2S)

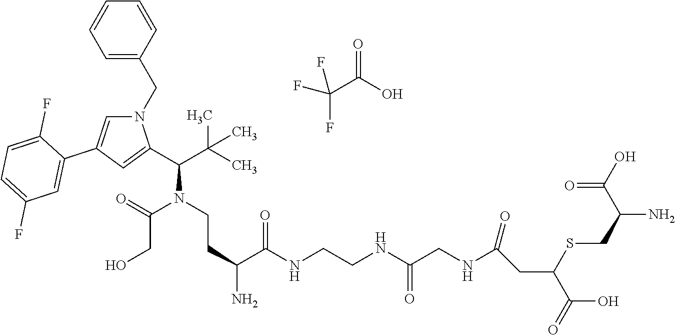 [SMILES: CC(C)(C)[C@H](c1cc(-c2cc(F)ccc2F)cn1Cc1ccccc1)N(CC[C@H](N)C(=O)NCCNC(=O)CNC(=O)CC(SC[C@H](N)C(=O)O)C(=O)O)C(=O)CO ; O=C(O)C(F)(F)F]

LC-MS (Method 5): Rₜ=2.44 min; MS (ESIpos): m/z=832 [M+H]⁺.

First, methyl L-cysteinate hydrochloride (1:1) was converted with 1-({[2-(trimethylsilyl)ethoxy]carbonyl}oxy)pyrrolidine-2,5-dione in DMF in the presence of N,N-diisopropylethylamine into methyl N-{[2-(trimethylsilyl)ethoxy]carbonyl}-L-cysteinate.

408 mg (1.93 mmol) of commercially available 3-bromo-4-methoxy-4-oxobutanoic acid and 180 mg (0.644 mmol) of methyl N-{[2-(trimethylsilyl)ethoxy]carbonyl}-L-cysteinate were dissolved in 8 ml of DMF, and 147 mg (0.97 mmol) of 1,8-diazabicyclo[5.4.0]undec-7-ene were added. After 18 h of stirring at RT, another 136 mg (0.64 mmol) of 3-bromo-4-methoxy-4-oxobutanoic acid and 147 mg (0.97 mmol) of 1,8-diazabicyclo[5.4.0]undec-7-ene were added, and the mixture was stirred at RT for a further 12 h and then concentrated under reduced pressure. The residue was purified by preparative HPLC. Combination of the appropriate fractions and evaporation of the solvents under reduced pressure gave 151 mg (57% of theory) of 4-methoxy-3-{[(2R)-3-methoxy-3-oxo-2-({[2-(trimethylsilyl)ethoxy]carbonyl}amino)propyl]sulphanyl}-4-oxobutanoic acid.

LC-MS (Method 12): Rₜ=1.74 min; MS (ESIneg): m/z=408 (M−H)⁻.

Of this intermediate, 145 mg were separated by supercritical fluid chromatography via chiral columns into the individual diastereomers (SFC; column: DAICEF, AD-H 5u 250×20 mm; flow rate: 80 ml/min; method: AD-25% ETOH-80 ml; pressure: 100 bar; wavelength: 210 nM), giving 63 mg (43%) of Epimer 1 and 58 mg (40%) of Epimer 2.

Epimer 1 was characterized as follows:

LC-MS (Method 5): Rₜ=2.94 min; MS (ESIneg): m/z=408 (M−H)⁻.

¹H-NMR: (400 MHz, DMSO-d₆): δ=7.57 (d, 1H), 4.24 (m, 1H), 4.05 (t, 2H), 3.67 (t, 1H), 3.65 (s, 3H), 3.62 (s, 3H), 3.05 (dd, 1H), 2.70-2.88 (m, 2H), 2.59 (dd, 1H), 0.93 (t, 2H), 0.02 (s, 9H).

Epimer 2 was characterized as follows:

LC-MS (Method 5): Rₜ=2.95 min; MS (ESIneg): m/z=408 (M−H)⁻.

¹H-NMR: (400 MHz, DMSO-d₆): δ=7.58 (d, 1H), 4.16-4.23 (m, 1H), 4.05 (t, 2H), 3.67 (dd, 1H), 3.65 (s, 3H), 3.64 (s, 3H), 3.04 (dd, 1H), 2.88 (dd, 1H), 2.77 (dd, 1H), 2.61 (dd, 1H), 0.92 (t, 2H), 0.02 (s, 9H).

32.5 mg (0.079 mmol) of Epimer 1 were coupled in the presence of 30 mg (0.079 mmol) of HATU and 13.4 mg (0.132 mmol) of 4-methylmorpholine with 50 mg (0.066 mmol) of Intermediate C66, giving, after HPLC purification, 43 mg (57% of theory) of the fully protected intermediate methyl 4-{[(8S)-8-{2-[{(1R)-1-[1-benzyl-4-(2,5-difluorophenyl)-1H-pyrrol-2-yl]-2,2-dimethylpropyl}(glycoloyl)amino]ethyl}-2,2-dimethyl-6,9,14-trioxo-5-oxa-7,10,13-triaza-2-silapentadecan-15-yl]amino}-2-{[(2R)-3-methoxy-3-oxo-2-({[2-(trimethylsilyl)ethoxy]carbonyl}amino)propyl]sulphanyl}-4-oxobutanoate.

40 mg (0.035 mmol) of this intermediate were then stirred at RT with 0.9 ml of a 2-molar lithium hydroxide solution in 11 ml of methanol for 20 min, resulting in the cleavage of both methyl ester groups. Purification by HPLC gave 12 mg (31% of theory) of the dicarboxylic acid derivative.

LC-MS (Method 5): Rₜ=4.74 min; MS (ESIpos): m/z=1120 [M+H]⁺.

Finally, 10 mg (0.009 mmol) of this intermediate were completely deprotected with zinc chloride in trifluoroethanol as described above. The residue was purified by preparative HPLC. Concentration of the appropriate fractions and lyophilization of the residue from acetonitrile/water gave 2.6 mg (30% of theory) of the title compound.

LC-MS (Method 5): Rₜ=2.44 min; MS (ESIpos): m/z=832 [M+H]⁺.

### Example M14

### 4-[(2-{[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)amino]-2-{[(2R)-2-amino-2-carboxyethyl]sulphanyl}-4-oxobutanoic acid/trifluoroacetic acid (1:1)

### Regioisomer 1, Epimer 2 (2R or 2S)

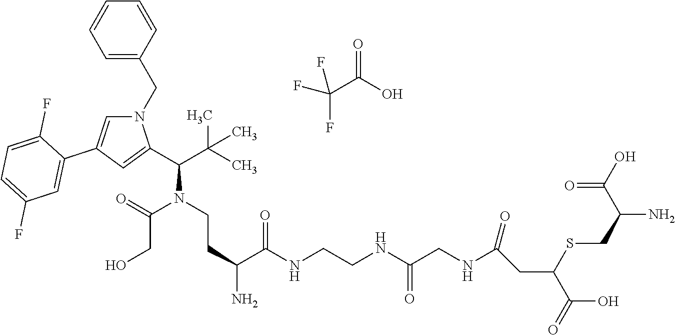 [SMILES: CC(C)(C)[C@H](c1cc(-c2cc(F)ccc2F)cn1Cc1ccccc1)N(CC[C@H](N)C(=O)NCCNC(=O)CNC(=O)CC(SC[C@H](N)C(=O)O)C(=O)O)C(=O)CO ; O=C(O)C(F)(F)F]

LC-MS (Method 5): Rₜ=2.44 min; MS (EIpos): m/z=832 [M+H]⁺.

The intermediate Epimer 2 described in Example M13 was reacted analogously to the description in Example M13:

32.5 mg (0.079 mmol) of Epimer 2 were coupled in the presence of 30 mg (0.079 mmol) of HATU and 13.4 mg (0.132 mmol) of 4-methylmorpholine with 50 mg (0.066 mmol) of Intermediate C66, giving, after HPLC purification, 43 mg (57% of theory) of the fully protected intermediate methyl 4-{[(8S)-8-{2-[{(1R)-1-[1-benzyl-4-(2,5-difluorophenyl)-1H-pyrrol-2-yl]-2,2-dimethylpropyl}(glycoloyl)amino]ethyl}-2,2-dimethyl-6,9,14-trioxo-5-oxa-7,10,13-triaza-2-silapentadecan-15-yl]amino}-2-{[(2R)-3-methoxy-3-oxo-2-({[2-(trimethylsilyl)ethoxy]carbonyl}amino)propyl]sulphanyl}-4-oxobutanoate. 40 mg (0.035 mmol) of this intermediate were then stirred at RT with 0.9 ml of a 2-molar lithium hydroxide solution in 11 ml of methanol for 20 min, resulting in the cleavage of both methyl ester groups. Purification by HPLC gave 11 mg (28% of theory) of the dicarboxylic acid derivative.

LC-MS (Method 5): Rₜ=4.74 min; MS (ESIpos): m/z=1120 [M+H]⁺.

Finally, 10 mg (0.009 mmol) of this intermediate were completely deprotected with zinc chloride in trifluoroethanol as described above. The residue was purified by preparative HPLC. Concentration of the appropriate fractions and lyophilization of the residue from acetonitrile/water gave 4.4 mg (52% of theory) of the title compound.

LC-MS (Method 5): Rₜ=2.44 min; MS (ESIpos): m/z=832 [M+H]⁺.

### Example M15

### 4-[(2-{[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)amino]-3-{[(2R)-2-amino-2-carboxyethyl]sulphanyl}-4-oxobutanoic acid/trifluoroacetic acid (1:1)

### Regioisomer 2, Epimer 1 (3R or 3S)

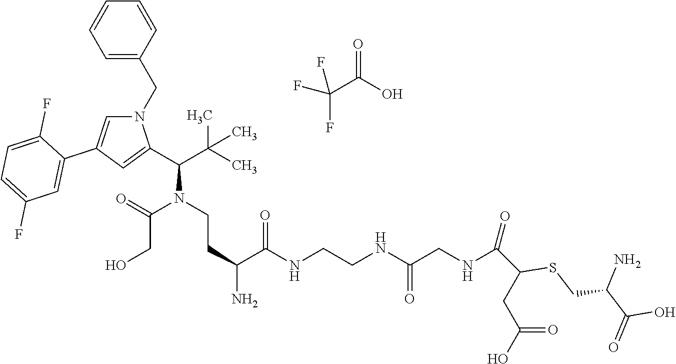 [SMILES: CC(C)(C)[C@H](c1cc(-c2cc(F)ccc2F)cn1Cc1ccccc1)N(CC[C@H](N)C(=O)NCCNC(=O)CNC(=O)C(CC(=O)O)SC[C@H](N)C(=O)O)C(=O)CO ; O=C(O)C(F)(F)F]

LC-MS (Method 5): Rₜ=2.45 min; MS (EIpos): m/z=832 [M+H]⁺.

742.8 mg (3.3 mmol) of commercially available 2-bromo-4-ethoxy-4-oxobutanoic acid and 802 mg (2.87 mmol) of methyl N-{[2-(trimethylsilyl)ethoxy]carbonyl}-L-cysteinate were dissolved in 32 ml of DMF, and 655.4 mg (4.31 mmol) of 1,8-diazabicyclo[5.4.0]undec-7-ene were added. After 20 h of stirring at RT, the reaction was concentrated under reduced pressure and the residue was purified by preparative HPLC. Combination of the appropriate fractions and evaporation of the solvents under reduced pressure gave 521 mg (43% of theory) of 4-ethoxy-2-{[(2R)-3-methoxy-3-oxo-2-({[2-(trimethylsilyl)ethoxy]carbonyl}amino)propyl]sulphanyl}-4-oxobutanoic acid.

LC-MS (Method 5): Rₜ=3.13 min; MS (ESIpos): m/z=424 (M+H)⁺.

Of this intermediate, 510 mg were separated by supercritical fluid chromatography via chiral columns into the individual diastereomers (SFC; column: DAICEL, AD-H 5u 250×20 mm; flow rate: 80 ml/min; method: AD-10% ETOH-80 ml; pressure: 100 bar; wavelength: 210 nM), giving 100 mg (20%) of Epimer 1 and 141 mg (28%) of Epimer 2.

Epimer 1 was characterized as follows:

LC-MS (Method 1): Rₜ=0.99 min; MS (ESIneg): m/z=422 (M−H)⁻.

¹H-NMR: (400 MHz, DMSO-d₆): δ=7.60 (d, 1H), 4.18-4.26 (m, 1H), 4.01-4.08 (m, 4H), 3.63 (s, 3H), 3.59 (dd, 1H), 3.04 (dd, 1H), 2.92 (dd, 1H), 2.80 (dd, 1H), 2.63 (dd, 1H), 1.17 (t, 3H), 0.92 (t, 2H), 0.02 (s, 9H).

Epimer 2 was characterized as follows:

LC-MS (Method 5): Rₜ=2.95 min; MS (ESIneg): m/z=408 (M−H)⁻.

¹H-NMR: (400 MHz, DMSO-d₆): δ=7.56 (d, 1H), 4.21-4.29 (m, 1H), 4.01-4.1 (m, 4H), 3.64 (s, 3H), 3.58 (dd, 1H), 3.08 (dd, 1H), 2.85 (dd, 1H), 2.78 (dd, 1H), 2.60 (dd, 1H), 1.17 (t, 3H), 0.93 (t, 2H), 0.02 (s, 9H).

33.6 mg (0.079 mmol) of Epimer 1 were coupled in the presence of 30 mg (0.079 mmol) of HATU and 13.4 mg (0.132 mmol) of 4-methylmorpholine with 50 mg (0.066 mmol) of Intermediate C66, giving, after HPLC purification, 51 mg (63% of theory) of the fully protected intermediate ethyl 4-{[(8S)-8-{2-[{(1R)-1-[1-benzyl-4-(2,5-difluorophenyl)-1H-pyrrol-2-yl]-2,2-dimethylpropyl}(glycoloyl)amino]ethyl}-2,2-dimethyl-6,9,14-trioxo-5-oxa-7,10,13-triaza-2-silapentadecan-15-yl]amino}-3-{[(2R)-3-methoxy-3-oxo-2-({[2-(trimethylsilyl)ethoxy]carbonyl}amino)propyl]sulphanyl}-4-oxobutanoate.

49 mg (0.042 mmol) of this intermediate were then stirred at RT with 0.5 ml of a 2-molar lithium hydroxide solution in 12 ml of THF/water 1:1 for 30 min, resulting in the cleavage of both methyl ester groups. Acidification and purification by HPLC gave 11 mg (24% of theory) of the dicarboxylic acid derivative.

LC-MS (Method 5): Rₜ=4.68 min; MS (ESIpos): m/z=1120 [M+H]⁺.

Finally, 11 mg (0.01 mmol) of this intermediate were completely deprotected with zinc chloride in trifluoroethanol as described above. The residue was purified by preparative HPLC. Concentration of the appropriate fractions and lyophilization of the residue from acetonitrile/water gave 3.7 mg (39% of theory) of the title compound.

LC-MS (Method 5): Rₜ=2.45 min; MS (ESIpos): m/z=832 [M+H]⁺.

### Example M16

### 4-[(2-{[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)amino]-3-{[(2R)-2-amino-2-carboxyethyl]sulphanyl}-4-oxobutanoic acid/trifluoroacetic acid (1:1)

### Regioisomer 2, Epimer 2 (3R or 3S)

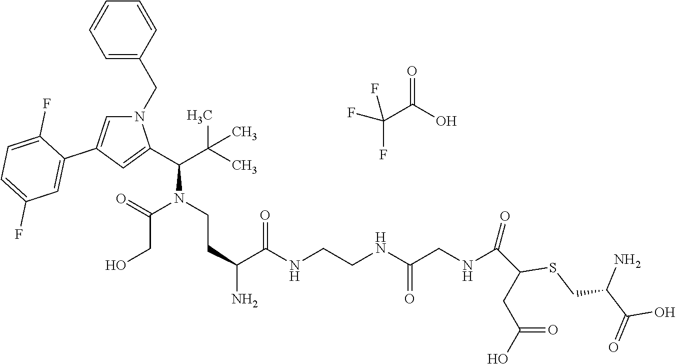 [SMILES: CC(C)(C)[C@H](c1cc(-c2cc(F)ccc2F)cn1Cc1ccccc1)N(CC[C@H](N)C(=O)NCCNC(=O)CNC(=O)C(CC(=O)O)SC[C@H](N)C(=O)O)C(=O)CO ; O=C(O)C(F)(F)F]

LC-MS (Method 5): Rₜ=2.44 min; MS (EIpos): m/z=832 [M+H]⁺.

The intermediate Epimer 2 described in Example M15 was reacted analogously to the description in Example M15:

33.6 mg (0.079 mmol) of Epimer 2 were coupled in the presence of 30 mg (0.079 mmol) of HATU and 13.4 mg (0.132 mmol) of 4-methylmorpholine with 50 mg (0.066 mmol) of Intermediate C66, giving, after HPLC purification, 51 mg (63% of theory) of the fully protected intermediate ethyl 4-{[(8S)-8-{2-[{(1R)-1-[1-benzyl-4-(2,5-difluorophenyl)-1H-pyrrol-2-yl]-2,2-dimethylpropyl}(glycoloyl)amino]ethyl}-2,2-dimethyl-6,9,14-trioxo-5-oxa-7,10,13-triaza-2-silapentadecan-15-yl]amino}-3-{[(2R)-3-methoxy-3-oxo-2-({[2-(trimethylsilyl)ethoxy]carbonyl}amino)propyl]sulphanyl}-4-oxobutanoate.

49 mg (0.042 mmol) of this intermediate were then stirred at RT with 0.5 ml of a 2-molar lithium hydroxide solution in 12 ml of THF/water 1:1 for 30 min, resulting in the cleavage of both methyl ester groups. Acidification and purification by HPLC gave 13.4 mg (28% of theory) of the dicarboxylic acid derivative.

LC-MS (Method 5): Rₜ=4.66 min; MS (ESIpos): m/z=1120 [M+H]⁺.

Finally, 13.4 mg (0.012 mmol) of this intermediate were completely deprotected with zinc chloride in trifluoroethanol as described above. The residue was purified by preparative HPLC. Concentration of the appropriate fractions and lyophilization of the residue from acetonitrile/water gave 7.5 mg (66% of theory) of the title compound.

LC-MS (Method 5): Rₜ=2.44 min; MS (ESIpos): m/z=832 [M+H]⁺.

### Example M17

### (2S)-2-Amino-4-[{(1R)-1-[1-benzyl-4-(2,5-difluorophenyl)-1H-pyrrol-2-yl]-2,2-dimethylpropyl}(glycoloyl)amino]butanoic acid hydrochloride (1:1)

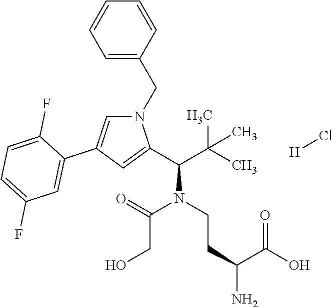 [SMILES: CC(C)(C)[C@H](c1cc(-c2cc(F)ccc2F)cn1Cc1ccccc1)N(CC[C@H](N)C(=O)O)C(=O)CO ; Cl]

150 mg (0.2 mmol) of Intermediate C53 were dissolved in 15 ml of DMF, and 2.29 g (20.39 mmol) of DABCO. The reaction was treated in an ultrasonic bath for 30 min. By addition of 1.17 ml of acetic acid, the reaction was then adjusted to pH 3-4, and the mixture was concentrated under reduced pressure. The residue was purified by preparative HPLC and the appropriate fractions were concentrated at RT under reduced pressure. The residue was taken up in acetonitrile/water (1:1), 5 ml of a 4N hydrochloric acid were added and the mixture was then lyophilized. This gave 81 mg (68% of theory) of the title compound.

LC-MS (Method 5): Rₜ=2.69 min; MS (EIpos): m/z=514 [M+H]⁺.

### Example M18

### N-[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]-L-glutamine/trifluoroacetic acid (1:1)

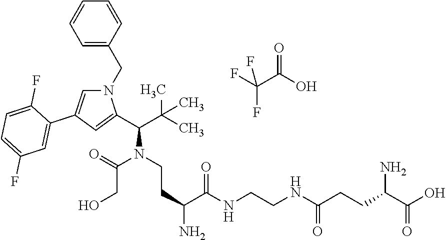 [SMILES: CC(C)(C)[C@H](c1cc(-c2cc(F)ccc2F)cn1Cc1ccccc1)N(CC[C@H](N)C(=O)NCCNC(=O)CC[C@H](N)C(=O)O)C(=O)CO ; O=C(O)C(F)(F)F]

First, trifluoroacetic acid/benzyl N-(2-aminoethyl)-N²-[(benzyloxy)carbonyl]-L-glutaminate (1:1) was prepared using classical methods of peptide chemistry. In the presence of HATU, this intermediate was then coupled with Intermediate C58. Subsequently, first the benzyloxycarbonyl protective group and the benzyl ester were removed by hydrogenolytic cleavage, and then the 2-(trimethylsilyl)ethoxycarbonyl protective group was removed using zinc chloride.

LC-MS (Method 6): Rₜ=1.91 min; MS (EIpos): m/z=685 [M+H]⁺.

### Example M19

### N⁶—(N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-beta-alanyl)-L-lysine/trifluoroacetic acid (1:1)

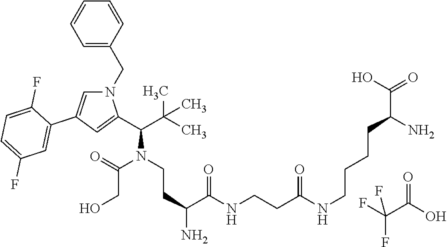

Initially, trifluoroacetic acid/2-(trimethylsilyl)ethyl-N2-[(benzyloxy)carbonyl]-L-lysinate (1:1) was prepared using classical protective group operations known in peptide chemistry. In the presence of HATU, this intermediate was then coupled with Intermediate C61. Subsequently, first the 2-(trimethylsilyl)ethoxycarbonyl protective group and the 2-(trimethylsilyl)ethyl ester were cleaved using zinc chloride. Finally, the title compound was obtained by hydrogenolytical cleavage of the benzyloxycarbonyl protective group and purification by preparative HPLC.

HPLC (Method 11): Rₜ=1.65 min;

### Example M20

### (1R,4R,27R,33R)-1-Amino-32-(3-aminopropyl)-33-[1-benzyl-4-(2,5-difluorophenyl)-1H-pyrrol-2-yl]-34,34-dimethyl-6,9,25,31-tetraoxo-13,16,19,22-tetraoxa-3,29-dithia-7,10,26,32-tetraazapentatriacontane-1,4,27-tricarboxylic acid/trifluoroacetic acid (1:2)

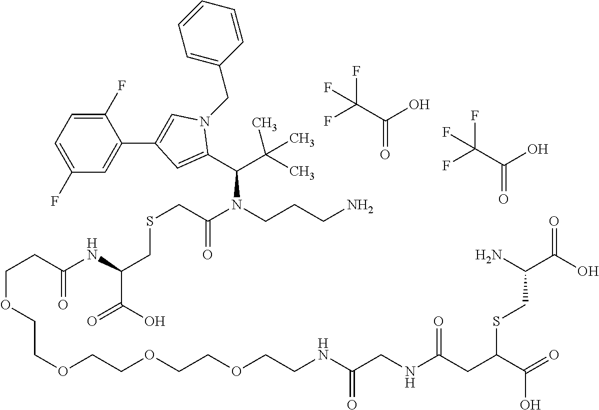

First, methyl L-cysteinate hydrochloride (1:1) was converted with 1-({[2-(trimethylsilyl)ethoxy]carbonyl}oxy)pyrrolidine-2,5-dione in DMF in the presence of N,N-diisopropylethylamine into methyl N-{[2-(trimethylsilyl)ethoxy]carbonyl}-L-cysteinate.

408 mg (1.93 mmol) of commercially available 3-bromo-4-methoxy-4-oxobutanoic acid and 180 mg (0.644 mmol) of methyl N-{[2-(trimethylsilyl)ethoxy]carbonyl}-L-cysteinate were dissolved in 8 ml of DMF, and 147 mg (0.97 mmol) of 1,8-diazabicyclo[5.4.0]undec-7-ene were added. After 18 h of stirring at RT, another 136 mg (0.64 mmol) of 3-bromo-4-methoxy-4-oxobutanoic acid and 147 mg (0.97 mmol) of 1,8-diazabicyclo[5.4.0]undec-7-ene were added, and the mixture was stirred at RT for a further 12 h and then concentrated under reduced pressure. The residue was purified by preparative HPLC. Combination of the appropriate fractions and evaporation of the solvents under reduced pressure gave 151 mg (57% of theory) of 4-methoxy-3-{[(2R)-3-methoxy-3-oxo-2-({[2-(trimethylsilyl)ethoxy]carbonyl}amino)propyl]sulphanyl}-4-oxobutanoic acid.

LC-MS (Method 12): Rₜ=1.74 min; MS (ESIneg): m/z=408 (M−H)⁻.

3.66 mg (8.93 μmol) of 4-methoxy-3-{[(2R)-3-methoxy-3-oxo-2-({[2-(trimethylsilyl)ethoxy]carbonyl}amino)propyl]sulphanyl}-4-oxobutanoic acid were coupled in the presence of 3.66 mg (8.93 μmol) of HATU and 1.6 μl (15 μmol) of 4-methylmorpholine with 13.0 mg (7.44 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[15-(glycylamino)-4,7,10,13-tetraoxapentadecan-1-oyl]-L-cysteine/trifluoroacetic acid (1:1) (Intermediate C80), giving, after HPLC purification, 3.9 mg (37% of theory) of the fully protected intermediate S-(11-{(1R)-1-[1-benzyl-4-(2,5-difluorphenyl)-1H-pyrrol-2-yl]-2,2-dimethylpropyl}-2,2-dimethyl-6,12-dioxo-5-oxa-7,11-diaza-2-silatridecan-13-yl)-N-[15-({N-[(8R,11R)-8,11-bis(methoxycarbonyl)-2,2-dimethyl-6,13-dioxo-5-oxa-10-thia-7-aza-2-silatridecan-13-yl]glycyl}amino)-4,7,10,13-tetraoxapentadecan-1-oyl]-L-cysteine.

3.90 mg (2.76 μmol) of this intermediate were then stirred at RT with 35 μl of a 2-molar lithium hydroxide solution in 1.0 ml of THF/water 3:1 for 15 min, resulting in the cleavage of both methyl ester groups. Purification by HPLC gave 3.60 mg (94% of theory) of the dicarboxylic acid derivative.

LC-MS (Method 5): Rₜ=4.83 min; MS (ESIpos): m/z=1385 [M+H]⁺.

Finally, 3.6 mg (2.6 μmol) of this intermediate were completely deprotected with zinc chloride in trifluoroethanol as described above. The residue was purified by preparative HPLC. Concentration of the appropriate fractions and lyophilization of the residue from acetonitrile/water gave 1.92 mg (55% of theory) of the title compound.

LC-MS (Method 5): Rₜ=2.72 min; MS (ESIneg): m/z=1094 [M−H]⁻.

### Example M21

### (2R,24S,27R)-27-Amino-2-[({2-[(3-aminopropyl) {(1R)-1-[1-benzyl-4-(2,5-difluorophenyl)-1H-pyrrol-2-yl]-2,2-dimethylpropyl}amino]-2-oxoethyl}sulphanyl)methyl]-24-(carboxymethyl)-4,20,23-trioxo-7,10,13,16-tetraoxa-25-thia-3,19,22-triazaoctacosane-1,28-dioic acid/trifluoroacetic acid (1:2)

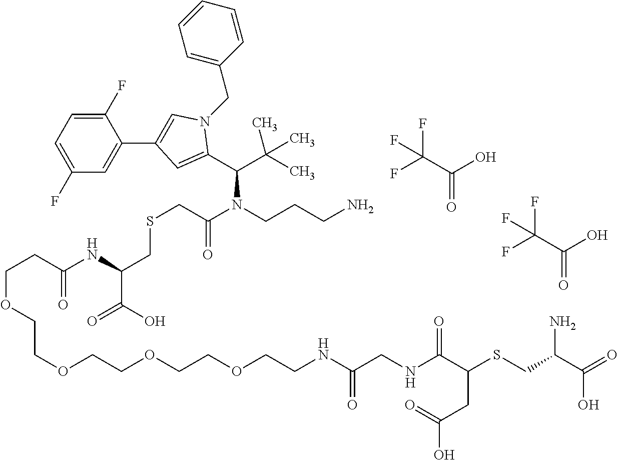

742.8 mg (3.3 mmol) of commercially available 2-bromo-4-ethoxy-4-oxobutanoic acid and 802 mg (2.87 mmol) of methyl N-{[2-(trimethylsilyl)ethoxy]carbonyl}-L-cysteinate were dissolved in 32 ml of DMF, and 655.4 mg (4.31 mmol) of 1,8-diazabicyclo[5.4.0]undec-7-ene were added. After 20 h of stirring at RT, the reaction was concentrated under reduced pressure and the residue was purified by preparative HPLC. Combination of the appropriate fractions and evaporation of the solvents under reduced pressure gave 521 mg (43% of theory) of 4-ethoxy-2-{[(2R)-3-methoxy-3-oxo-2-({[2-(trimethylsilyl)ethoxy]carbonyl}amino)propyl]sulphanyl}-4-oxobutanoic acid.

LC-MS (Method 5): Rₜ=3.13 min; MS (ESIpos): m/z=424 (M+H)⁺.

4.36 mg (10.3 μmol) of 4-ethoxy-2-{[(2R)-3-methoxy-3-oxo-2-({[2-(trimethylsilyl)ethoxy]carbonyl}amino)propyl]sulphanyl}-4-oxobutanoic acid were coupled in the presence of 3.92 mg (10.3 μmol) of HATU and 1.9 μl (17 μmol) of 4-methylmorpholine with 15.0 mg (8.59 μmol) of S-(11-{(1R)-1-[1-benzyl-4-(2,5-difluorophenyl)-1H-pyrrol-2-yl]-2,2-dimethylpropyl}-2,2-dimethyl-6,12-dioxo-5-oxa-7,11-diaza-2-silatridecan-13-yl)-N-[15-(glycylamino)-4,7,10,13-tetraoxapentadecan-1-oyl]-L-cysteine/trifluoroacetic acid (1:1) (Intermediate C80), giving, after HPLC purification, 3.6 mg (26% of theory) of the fully protected intermediate S-(11-{(1R)-1-[1-benzyl-4-(2,5-difluorphenyl)-1H-pyrrol-2-yl]-2,2-dimethylpropyl}-2,2-dimethyl-6,12-dioxo-5-oxa-7,11-diaza-2-silatridecan-13-yl)-N-[15-({N-[(8R,11S)-11-(2-ethoxy-2-oxoethyl)-8-(methoxycarbonyl)-2,2-dimethyl-6,12-dioxo-5-oxa-10-thia-7-aza-2-siladodecan-12-yl]glycyl}amino)-4,7,10,13-tetraoxapentadecan-1-oyl]-L-cysteine. 6.20 mg (2.82 μmol) of this intermediate were then stirred at RT with 35 μl of a 2-molar lithium hydroxide solution in 1.0 ml of THF/water 1:1 for 15 min, resulting in the cleavage of both ester groups. Acidification and purification by HPLC gave 3.60 mg (92% of theory) of the dicarboxylic acid derivative.

LC-MS (Method 5): Rₜ=4.71 min; MS (ESIpos): m/z=1385 [M+H]⁺.

Finally, 3.60 mg (1.69 μmol) of this intermediate were completely deprotected with zinc chloride in trifluoroethanol as described above. The residue was purified by preparative HPLC. Concentration of the appropriate fractions and lyophilization of the residue from acetonitrile/water gave 0.88 mg (39% of theory) of the title compound.

LC-MS (Method 5): Rₜ=2.72 min; MS (ESIneg): m/z=1094 [M−H]⁻.

### Example M22

### (2R,27R)-27-Amino-2-[({2-[(3-aminopropyl) {(1R)-1-[1-benzyl-4-(2,5-difluorophenyl)-1H-pyrrol-2-yl]-2,2-dimethylpropyl}amino]-2-oxoethyl}sulphanyl)methyl]-24-(carboxymethyl)-4,20,23-trioxo-7,10,13,16-tetraoxa-25-thia-3,19,22-triazaoctacosane-1,28-dioic acid/trifluoroacetic acid (1:2) and

### (1R,27R,33R)-1-amino-32-(3-aminopropyl)-33-[1-benzyl-4-(2,5-difluorophenyl)-1H-pyrrol-2-yl]-34,34-dimethyl-6,9,25,31-tetraoxo-13,16,19,22-tetraoxa-3,29-dithia-7,10,26,32-tetraazapentatriacontane-1 N,N-tricarboxylic acid/trifluoroacetic acid (1:2)

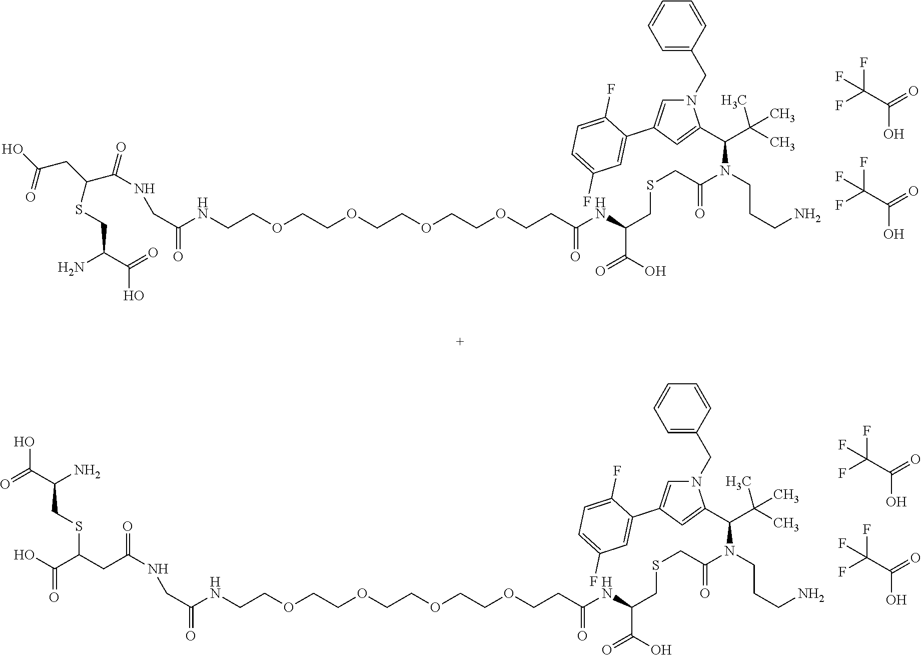

16.5 mg (0.015 mmol) of S-{2-[(3-aminopropyl) {(1R)-1-[1-benzyl-4-(2,5-difluorophenyl)-1H-pyrrol-2-yl]-2,2-dimethylpropyl}amino]-2-oxoethyl}-N-[1-(2,5-dioxo-2,5-dihydro-1H-pyrrol-1-yl)-2,18-dioxo-6,9,12,15-tetraoxa-3-azaoctadecan-18-yl]-L-cysteine/trifluoroacetic acid (1:1) (intermediate F257) and 8.18 mg (0.031 mmol) of N-{[2-(trimethylsilyl)ethoxy]carbonyl}-L-cysteine were dissolved in 2 ml of DMF, and the mixture was stirred at RT for 18 h. The reaction mixture was concentrated under reduced pressure. The residue (28.9 mg) was dissolved in 3 ml of THF/water 1:1. 0.046 ml of a 2M aqueous lithium hydroxide solution was added and the mixture was stirred at RT for 3 hours. The mixture was then adjusted to a pH of 7 using 5.2 μl (0.092 mmol) of acetic acid. The reaction mixture was purified directly by prep. RP-HPLC (column: Reprosil 125×30; 10μ, flow rate: 50 ml/min, MeCN/water; 0.1% TFA). The solvents were evaporated under reduced pressure and the residue was dried under high vacuum. This gave 12.1 mg (58% over 2 steps) of the regioisomeric protected intermediates.

LC-MS (Method 12): Rₜ=1.82 min; MS (ESIpos): m/z=1240 (M+H)⁺.

In the last step, 12.1 mg (0.009 mmol) of this intermediate were dissolved in 2 ml of 2,2,2-trifluoroethanol. 7.3 mg (0.054 mmol) of zinc chloride were added, and the mixture was stirred at 50° C. for 2 h. 15.7 mg (0.054 mmol) of ethylenediamine-N,N,N′,N′-tetraacetic acid were then added, and the solvent was purified by preparative HPLC. Concentration of the appropriate fractions and lyophilization of the residue from acetonitrile/water gave 6.4 mg (59%) of the title compound as a regioisomer mixture.

LC-MS (Method 1): Rₜ=0.86 min; MS (ESIpos): m/z=1096 (M+H)⁺.

### Example M23

### N-{(2S)-2-Amino-4-[{(1R)-1-[1-benzyl-4-(2,5-difluorophenyl)-1H-pyrrol-2-yl]-2,2-dimethyl propyl}(glycoloyl)amino]butanoyl}-beta-alanyl-L-glutamic acid/trifluoro acetic acid (1:1)

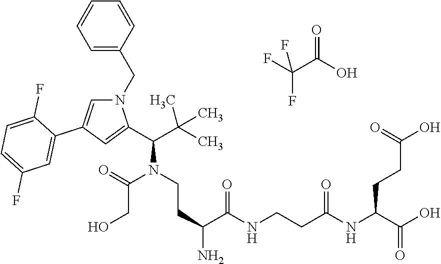 [SMILES: CC(C)(C)[C@H](c1cc(-c2cc(F)ccc2F)cn1Cc1ccccc1)N(CC[C@H](N)C(=O)NCCC(=O)N[C@@H](CCC(=O)O)C(=O)O)C(=O)CO ; O=C(O)C(F)(F)F]

First, di-tert-butyl L-glutamate hydrochloride (1:1) was coupled with intermediate C61 in the presence of HATU and N,N-diisopropylethylamine. The protected intermediate was then taken up in trifluoroethanol and, by stirring at 50° C. in the presence of zinc chloride overnight, deprotected completely. Work-up was carried out after addition of EDTA by purification by preparative HPLC.

LC-MS (Method 12): Rₜ=1.45 min; MS (ESIpos): m/z=714 [M+H]⁺.

### Example M24

### N-{(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}-beta-alanyl-D-glutamic acid/trifluoro acetic acid (1:1)

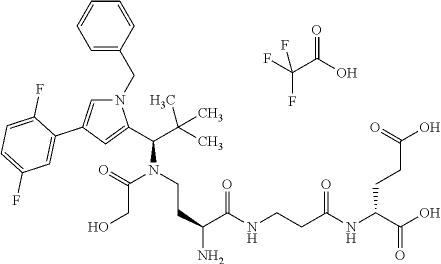 [SMILES: CC(C)(C)[C@H](c1cc(-c2cc(F)ccc2F)cn1Cc1ccccc1)N(CC[C@H](N)C(=O)NCCC(=O)N[C@H](CCC(=O)O)C(=O)O)C(=O)CO ; O=C(O)C(F)(F)F]

First, di-tert-butyl D-glutamate hydrochloride (1:1) was coupled with intermediate C61 in the presence of HATU and N,N-diisopropylethylamine. The protected intermediate was then taken up in trifluoroethanol and, by stirring at 50° C. in the presence of zinc chloride, deprotected completely. Work-up was carried out after addition of EDTA by purification by preparative HPLC.

LC-MS (Method 12): Rₜ=1.41 min; MS (ESIpos): m/z=714 [M+H]⁺.

### Example M25

### N-{(2S)-2-Amino-4-[{(1R)-1-[1-benzyl-4-(2,5-difluorophenyl)-1H-pyrrol-2-yl]-2,2-dimethyl propyl}(glycoloyl)amino]butanoyl}-L-glutamic acid/trifluoroacetic acid (1:1)

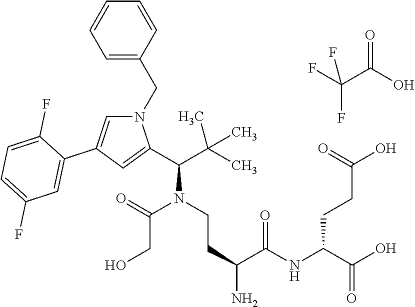

First, di-tert-butyl L-glutamate hydrochloride (1:1) was coupled with intermediate C61 in the presence of HATU and N,N-diisopropylethylamine. In the next step, the Z protective group was removed by hydrogenation over 10% palladium on activated carbon in methanol at RT under standard hydrogen pressure for 45 minutes. The partially protected intermediate was then taken up in trifluoroethanol and deprotected completely by stirring at 50° C. in the presence of zinc chloride for 7 hours. Work-up was carried out after addition of EDTA by purification by preparative HPLC.

LC-MS (Method 12): Rₜ=1.44 min; MS (ESIpos): m/z=643 [M+H]⁺.

### Example M26

### 4-[(2-{[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)amino]-2-{[(2R)-2-amino-2-carboxyethyl]sulphanyl}-4-oxobutanoic acid/trifluoro acetic acid (1:1)

### Regioisomer 1, Mixture of Epimers

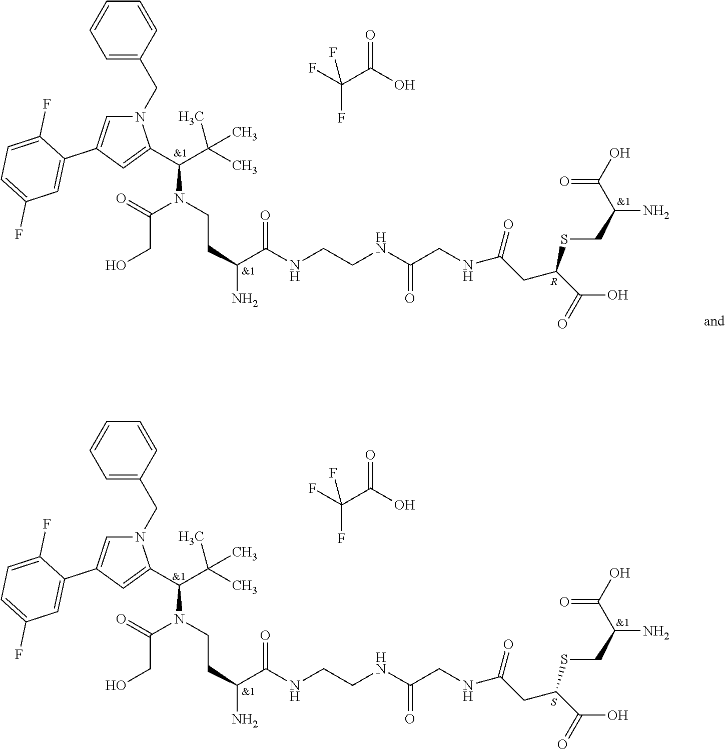

This example describes the epimer mixture of the compounds of Example 13 and Example 14. The synthesis was carried out analogously to Example 13, where the separation of the two epimers by supercritical fluid chromatography was dispensed with and the title compound was prepared as a mixture of epimers.

LC-MS (Method 5): Rₜ=2.43 min; MS (ESIpos): m/z=832 [M+H]⁺.

### Example M27

### 4-[(2-{[2-({(2S)-2-Amino-4-[{(1R)-1-[1-benzyl-4-(2,5-difluorophenyl)-1H-pyrrol-2-yl]-2,2-dimethylpropyl}(glycoloyl)amino]butanoyl}amino)ethyl]amino}-2-oxoethyl)amino]-3-{[(2R)-2-amino-2-carboxyethyl]sulphanyl}-4-oxobutanoic acid/trifluoro acetic acid (1:1)

### Regioisomer 2, Mixture of Epimers

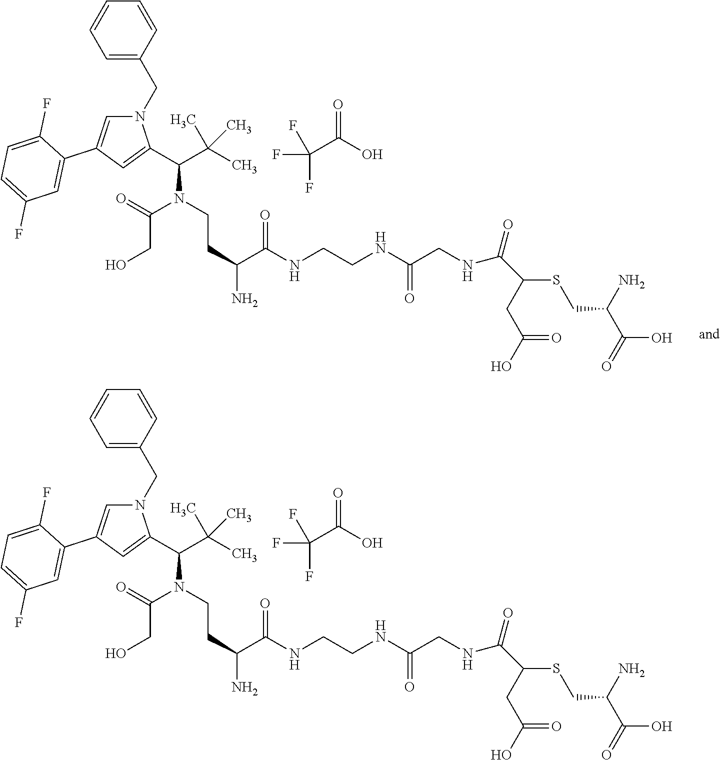

This example describes the epimer mixture of the compounds of Example 15 and Example 16. The synthesis was carried out analogously to Example 15, where the separation of the two epimers by supercritical fluid chromatography was dispensed with and the title compound was prepared as a mixture of epimers.

LC-MS (Method 5): Rₜ=2.45 min; MS (EIpos): m/z=832 [M+H]⁺.

### WORKING EXAMPLES ADCS

The ADCs shown in the structural formulae of the Working examples, which were coupled to the cystein side chains of the antibodies via maleimide radicals, are, depending on the linker and the coupling procedure, mainly present in the ring-opened or ring-closed forms shown in each case. However, the preparation may comprise a small proportion of the respective other form.

### Example 194N

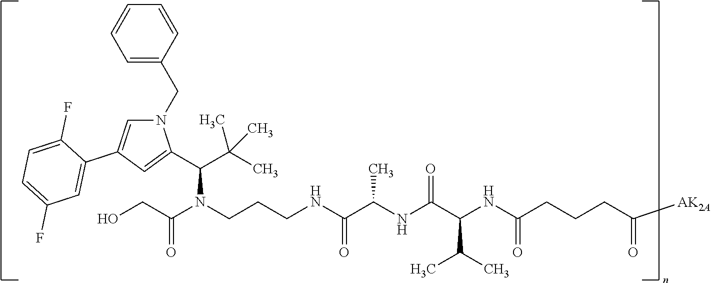

Here, 2.5 mg of anti-TWEAKR AK_{1A} (ITEM-4) in PBS (c=10 mg/ml) were used for coupling with Intermediate F194. First, 4 eq of Intermediate F194 dissolved in 25 μl of DMSO were added, and after 1 h of stirring at RT the same amount was added again and the reaction was stirred at RT for a further hour. The reaction was subsequently purified on a Sephadex column, then concentrated by ultracentrifugation and rediluted with PBS.

Protein concentration: 1.22 mg/ml

Drug/mAb ratio: 2.8

### Example 208N

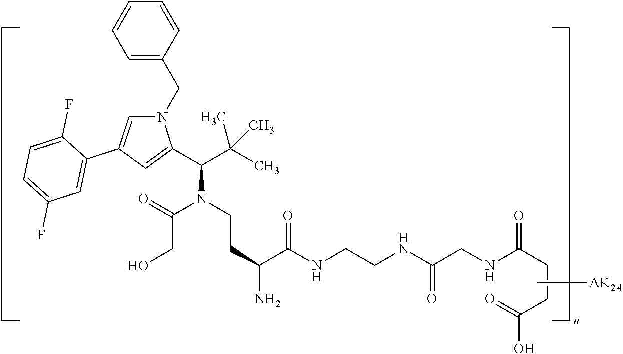

Under argon, a solution of 0.029 mg of TCEP in 50 μl of PBS buffer was added to 5 mg of anti-TWEAKR AK_{1A} (ITEM-4) in 0.450 ml of PBS (c=11.1 mg/ml). The reaction was stirred at RT for 30 min, and 0.19 mg (0.00023 mmol) of Intermediate F104 dissolved in 50 μl of DMSO were then added. After a further 90 min of stirring at RT, the reaction was made up to 2.5 ml with PBS buffer pH 8 and applied to a PD 10 column (Sephadex® G-25, GE Healthcare) which had been equilibrated with PBS buffer pH 8, eluted with PBS buffer pH 8 and then stirred under argon at RT overnight. The eluate was then concentrated by ultracentrifugation and rediluted with PBS buffer (pH 7.2). The ADC batch obtained was characterized as follows:

Protein concentration: 1.78 mg/ml

Drug/mAb ratio: 1.7

Under argon, a solution of 0.287 mg of TCEP in 0.5 ml of PBS buffer was added to 50 mg of anti-TWEAKR AK_{1A} (ITEM-4) in 5 ml of PBS (c=10 mg/ml). The reaction was stirred at RT for 30 min, and 2.15 mg (0.00267 mmol) of Intermediate F104 dissolved in 500 μl of DMSO were then added. After a further 90 min of stirring at RT, the reaction was diluted with 4 ml of PBS buffer which had been adjusted to pH 8 beforehand, and stirred under argon at RT overnight. The solution was then re-buffered with PBS buffer to a pH of 7.2 using PD-10 columns. The eluate was then concentrated by ultracentrifugation, rediluted with PBS buffer (pH 7.2), and concentrated again. The ADC batch obtained was characterized as follows:

Protein concentration: 16.2 mg/ml

Drug/mAb ratio: 1.4

### Example 208O

Under argon, a solution of 0.4 mg of TCEP in 0.6 ml of PBS buffer was added to 70 mg of anti-TWEAKR AK_{1C} (TPP-7006) in 7.4 ml of PBS (c=9.5 mg/ml). The mixture was stirred at RT for 30 min, and 2.64 mg (0.00327 mmol) of Intermediate F104, dissolved in 800 μl of DMSO, were then added. After a further 90 min of stirring at RT, the mixture was diluted with 1.2 ml of PBS buffer, which had been adjusted to pH 8 beforehand, and then rebuffered to pH 8 using PD-10 columns. The combined eluates were diluted with PBS buffer (pH 8) to a total volume of 15 ml and then stirred under argon at RT overnight.

The mixture was then rebuffered to a pH of 7.2 using PD-10 columns with PBS buffer. The eluate was then concentrated by ultracentrifugation, rediluted with PBS buffer (pH 7.2) and concentrated again. The ADC batch obtained was characterized as follows:

Protein concentration: 12.2 mg/ml

Drug/mAb ratio: 2.7

For this ADC preparation, the proportion of the ring-opened succinamide form was determined to be 84.1%.

### Example 208P

Under argon, a solution of 0.4 mg of TCEP in 0.6 ml of PBS buffer was added to 70 mg of anti-TWEAKR AK_{1B} (TPP-7005) in 7.4 ml of PBS (c=9.5 mg/ml). The mixture was stirred at RT for 30 min, and 2.64 mg (0.00327 mmol) of Intermediate F104, dissolved in 800 μl of DMSO, were then added. After a further 90 min of stirring at RT, the mixture was diluted with 1.2 ml of PBS buffer, which had been adjusted to pH 8 beforehand, and then rebuffered to pH 8 using PD-10 columns. The combined eluates were diluted with PBS buffer (pH 8) to a total volume of 15 ml and then stirred under argon at RT overnight.

The mixture was then rebuffered to a pH of 7.2 using PD-10 columns with PBS buffer. The eluate was then concentrated by ultracentrifugation, rediluted with PBS buffer (pH 7.2) and concentrated again. The ADC batch obtained was characterized as follows:

Protein concentration: 10.99 mg/ml

Drug/mAb ratio: 2.4

For this ADC preparation, the proportion of the ring-opened succinamide form was determined to be 84.4%.

### Example 208Q

Under argon, a solution of 0.4 mg of TCEP in 0.6 ml of PBS buffer was added to 70 mg of anti-TWEAKR AK_{1D} (TPP-7007) in 7.4 ml of PBS (c=9.5 mg/ml). The mixture was stirred at RT for 30 min, and 2.64 mg (0.00327 mmol) of Intermediate FI 04, dissolved in 800 μl of DMSO, were then added. After a further 90 min of stirring at RT, the mixture was diluted with 1.2 ml of PBS buffer, which had been adjusted to pH 8 beforehand, and then rebuffered to pH 8 using PD-10 columns. The combined eluates were diluted with PBS buffer (pH 8) to a total volume of 15 ml and then stirred under argon at RT overnight.

The mixture was then rebuffered to a pH of 7.2 using PD-10 columns with PBS buffer. The eluate was then concentrated by ultracentrifugation, rediluted with PBS buffer (pH 7.2) and concentrated again. The ADC batch obtained was characterized as follows:

Protein concentration: 12.01 mg/ml

Drug/mAb ratio: 2.8

For this ADC preparation, the proportion of the ring-opened succinamide form was determined to be 85.2%.

### Example 240O

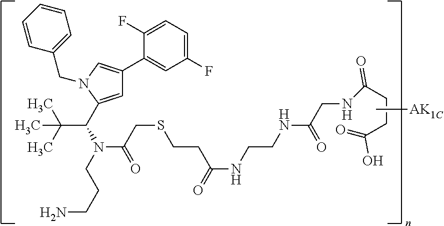

Here, 5 mg of anti-TWEAKR AK_{1C} (TPP-7006) in 450 μl of PBS were used for coupling with intermediate F240 at pH 7.2 (c=11.1 mg/ml). The reduction time of the antibody in the presence of 0.029 mg of TCEP was 30 min. 0.20 mg (0.23 μmol) of F240 in 50 μl of DMSO was then added, and the mixture was stirred at RT for a further 90 min. The mixture was then made up to 2.5 ml using PBS buffer (pH 8) and applied to a PD10 column equilibrated (Sephadex® G-25, GE Healthcare) with PBS buffer pH 8, was eluted with PBS buffer pH 8 and was then stirred under argon at RT overnight. The eluate was subsequently concentrated by ultracentifugation and rediluted with PBS buffer (pH 7.2). The ADC batch obtained was characterized as follows:

Protein concentration: 1.55 mg/ml

Drug/mAb ratio: 3.8

### Example 257N

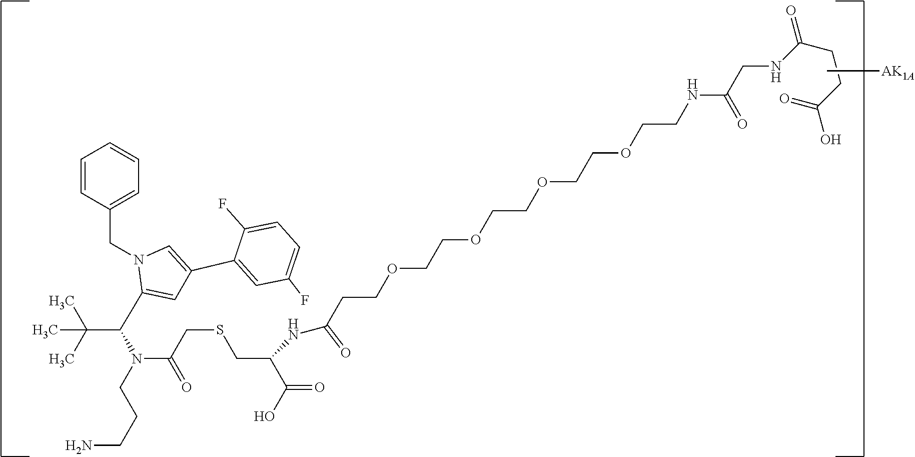

Under argon, a solution of 0.029 mg of TCEP in 50 μl of PBS buffer was added to 5 mg of anti-TWEAKR AK_{1A} (ITEM-4) in 319 μl of PBS (c=15.7 mg/ml). The reaction was diluted with 2031 μl of PBS buffer which had been adjusted to pH 8 beforehand and stirred at RT for 1 h. 0.250 mg (0.00023 mmol) of Intermediate F257 dissolved in 100 μl of DMSO were then added. After a further 90 min of stirring at RT, the reaction was applied to PD 10 columns (Sephadex® G-25, GE Healthcare) which had been equilibrated with PBS buffer pH 8 and was eluted with PBS buffer pH 8. The eluate was stirred under argon at RT overnight and then concentrated by ultracentrifugation and rediluted with PBS buffer (pH 7.2). Under these conditions, some of the ADCs may also be present in the ring-closed form. The ADC batch obtained was characterized as follows:

Protein concentration: 1.44 mg/ml

Drug/mAb ratio: 1.0

### Example 257P

Here, 5 mg of anti-TWEAKR AK_{1B} (TPP-7005) in 450 μl of PBS were used for coupling with intermediate F257 at pH 7.2 (c=11.1 mg/ml). The reduction time of the antibody in the presence of 0.029 mg of TCEP was 30 min. 0.20 mg (0.23 μmol) of F257 in 50 μl of DMSO was then added, and the mixture was stirred at RT for a further 90 min. The mixture was then made up to 2.5 ml using PBS buffer (pH 8) and applied to a PD10 column equilibrated (Sephadex® G-25, GE Healthcare) with PBS buffer pH 8, was eluted with PBS buffer pH 8 and was then stirred under argon at RT overnight. The eluate was subsequently concentrated by ultracentifugation and rediluted with PBS buffer (pH 7.2). The ADC batch obtained was characterized as follows:

Protein concentration: 1.67 mg/ml

Drug/mAb ratio: 4.7

### Example 260N

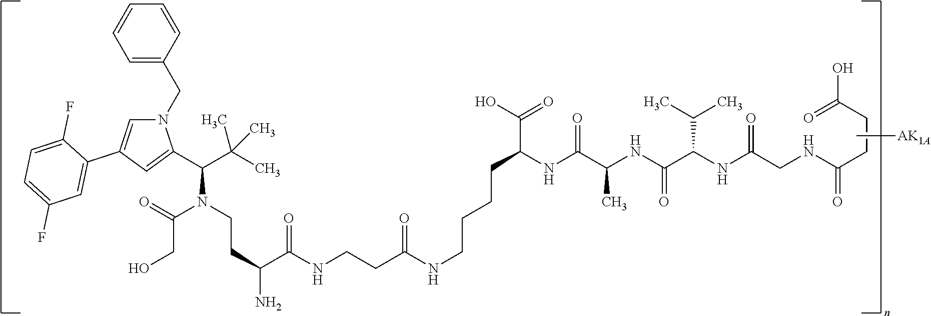

Under argon, a solution of 0.014 mg of TCEP in 25 μl of PBS buffer was added to 2.5 mg of anti-TWEAKR AK_{1A} (ITEM-4) in 250 μl of PBS (c=10 mg/ml). The reaction was stirred at RT for 30 min, and 0.151 mg (0.00013 mmol) of Intermediate F260 dissolved in 25 μl of DMSO were then added. After a further 90 min of stirring at RT, the reaction was diluted with 2200 μl of PBS buffer which had been adjusted to pH 8 beforehand.

This solution was then applied to PD 10 columns (Sephadex® G-25, GE Healthcare) which had been equilibrated with PBS buffer pH 8 and was eluted with PBS buffer pH 8. The eluate was stirred under argon at RT overnight and then concentrated by ultracentrifugation and rediluted with PBS buffer (pH 7.2). Under these conditions, some of the ADCs may also be present in the ring-closed form. The ADC batch obtained was characterized as follows:

Protein concentration: 1.49 mg/ml

Drug/mAb ratio: 2.2

### Example 274N

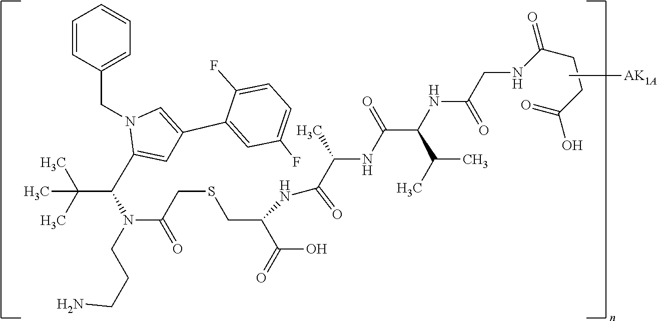

Under argon, a solution of 0.014 mg of TCEP in 25 μl of PBS buffer was added to 2.5 mg of anti-TWEAKR AK_{1A} (ITEM-4) in 208 μl of PBS (c=12.0 mg/ml). The reaction was diluted with 967 μl of PBS buffer which had been adjusted to pH 8 beforehand and stirred at RT for 1 h. 0.116 mg (0.00012 mmol) of Intermediate F274 dissolved in 50 μl of DMSO were then added. After a further 90 min of stirring at RT, the reaction was applied to PD 10 columns (Sephadex® G-25, GE Healthcare) which had been equilibrated with PBS buffer pH 8 and was eluted with PBS buffer pH 8. The eluate was stirred under argon at RT overnight and then concentrated by ultracentrifugation and rediluted with PBS buffer (pH 7.2). Under these conditions, some of the ADCs may also be present in the ring-closed form. The ADC batch obtained was characterized as follows:

Protein concentration: 1.31 mg/ml

Drug/mAb ratio: 1.5

### Example 275N

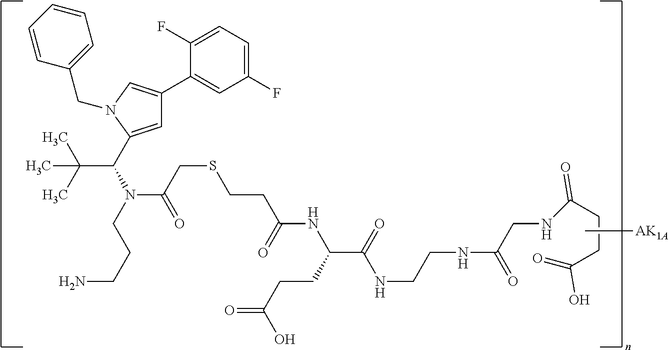

Under argon, a solution of 0.014 mg of TCEP in 25 μl of PBS buffer was added to 2.5 mg of anti-TWEAKR AK_{1A} (ITEM-4) in 208 μl of PBS (c=12.0 mg/ml). The reaction was diluted with 967 μl of PBS buffer which had been adjusted to pH 8 beforehand and stirred at RT for 1 h. 0.116 mg (0.00012 mmol) of Intermediate F275 dissolved in 50 μl of DMSO were then added. After a further 90 min of stirring at RT, the reaction was applied to PD 10 columns (Sephadex® G-25, GE Healthcare) which had been equilibrated with PBS buffer pH 8 and was eluted with PBS buffer pH 8. The eluate was stirred under argon at RT overnight and then concentrated by ultracentrifugation and rediluted with PBS buffer (pH 7.2). Under these conditions, some of the ADCs may also be present in the ring-closed form. The ADC batch obtained was characterized as follows:

Protein concentration: 1.29 mg/ml

Drug/mAb ratio: 1.8

### Example 281N

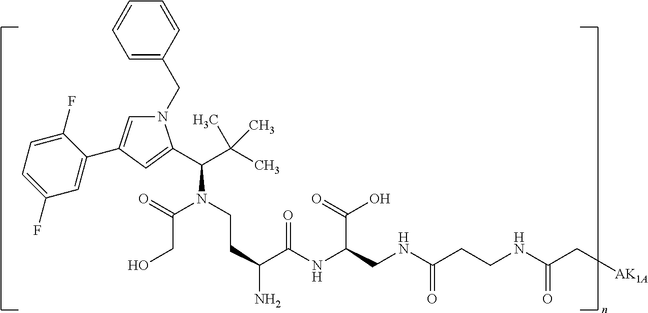

Here, 2.5 mg of anti-TWEAKR AK_{1A} (ITEM-4) in 208 μl of PBS at pH 7.2 (c=12 mg/ml) were used for coupling with Intermediate F281. The reduction time of the antibody in the presence of 0.014 mg of TCEP was 30 min. After addition of 0.11 mg (0.12 μmol) of F281 in 25 μl of DMSO, the reaction was then stirred at RT for 20 h and subsequently purified on Sephadex. The eluate was finally concentrated by ultracentrifugation and rediluted with PBS.

Protein concentration: 0.59 mg/ml

Drug/mAb ratio: 1.3

### Example 281P

Here, 5 mg of anti-TWEAKR AK_{1B} (TPP-7005) in 500 μl of PBS were used for coupling with intermediate F281 at pH 7.2 (c=10 mg/ml). The reduction time of the antibody in the presence of 0.029 mg of TCEP was 30 min. The mixture was then, after addition of 0.25 mg (0.27 μmol) of F281 in 50 μl of DMSO, stirred at RT for 20 h and subsequently purified on Sephadex. Finally, the mixture was concentrated by ultracentrifugation and rediluted with PBS.

Protein concentration: 1.9 mg/ml

Drug/mAb ratio: 3.4

### Example 281Q

Here, 5 mg of anti-TWEAKR AK_{1D} (TPP-7007) in 500 μl of PBS were used for coupling with intermediate F281 at pH 7.2 (c=10 mg/ml). The reduction time of the antibody in the presence of 0.029 mg of TCEP was 30 min. The mixture was then, after addition of 0.25 mg (0.27 μmol) of F281 in 50 μl of DMSO, stirred at RT for 20 h and subsequently purified on Sephadex. Finally, the reaction was concentrated by ultracentrifugation and rediluted with PBS.

Protein concentration: 2.55 mg/ml

Drug/mAb ratio: 2.8

### Example 284N

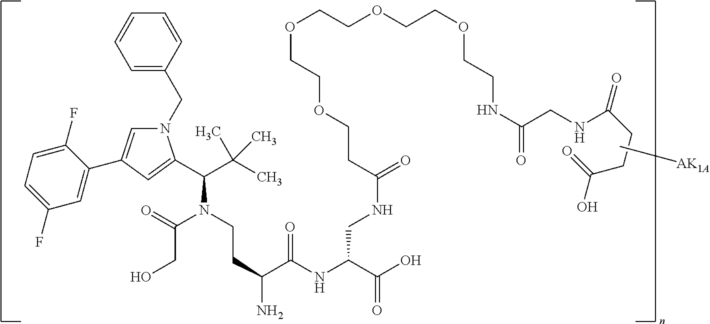

Under argon, a solution of 0.029 mg of TCEP in 50 μl of PBS buffer was added to 5 mg of anti-TWEAKR AK_{1A} (ITEM-4) in 417 μl of PBS (c=12 mg/ml), and the mixture was stirred at RT for 45 min. 0.29 mg (0.7 μmol) of Intermediate F284 dissolved in 50 μl of DMSO were then added. After a further 90 min of stirring at RT, the mixture was made up to 2.5 ml with PBS buffer pH 8 and passed through a PD 10 column (Sephadex® G-25, GE Healthcare) equilibrated with PBS buffer pH 8, eluted with PBS buffer pH 8 and then stirred under argon at RT overnight. The eluate was then concentrated by ultracentrifugation and rediluted with PBS buffer (pH 7.2). The ADC batch obtained was characterized as follows:

Protein concentration: 1.81 mg/ml

Drug/mAb ratio: 2.0

### Example 284O

Here, 5 mg of anti-TWEAKR AK_{1C} (TPP-7006) in 400 μl of PBS were used for coupling with intermediate F284 at pH 7.2 (c=12.5 mg/ml). The reduction time of the antibody in the presence of 0.029 mg of TCEP was 30 min. 0.26 mg (0.23 μmol) of F284 in 50 μl of DMSO were then added and the reaction was stirred at RT for a further 90 min. The mixture was then made up to 2.5 ml with PBS buffer (pH 8) and applied to a PD-10 column (Sephadex® G-25, GE Healthcare) equilibrated with PBS buffer pH 8, was eluted with PBS buffer pH 8 and was then stirred under argon at RT overnight. The eluate was then concentrated by ultracentrifugation and rediluted with PBS buffer (pH 7.2). The ADC batch obtained was characterized as follows:

Protein concentration: 1.88 mg/ml

Drug/mAb ratio: 3.1

### Example 284P

Here, 5 mg of anti-TWEAKR AK_{1B} (TPP-7005) in 450 μl of PBS were used for coupling with intermediate F284 at pH 7.2 (c=11.1 mg/ml). The reduction time of the antibody in the presence of 0.029 mg of TCEP was 30 min. 0.26 mg (0.23 μmol) of F284 in 50 μl of DMSO were then added and the mixture was stirred at RT for a further 90 min. The mixture was then made up to 2.5 ml with PBS buffer (pH 8) and applied to a PD-10 column (Sephadex® G-25, GE Healthcare) equilibrated with PBS buffer pH 8, was eluted with PBS buffer pH 8 and was then stirred under argon at RT overnight. The eluate was then concentrated by ultracentrifugation and rediluted with PBS buffer (pH 7.2). The ADC batch obtained was characterized as follows:

Protein concentration: 2.11 mg/ml

Drug/mAb ratio: 2.5

### Example 294N

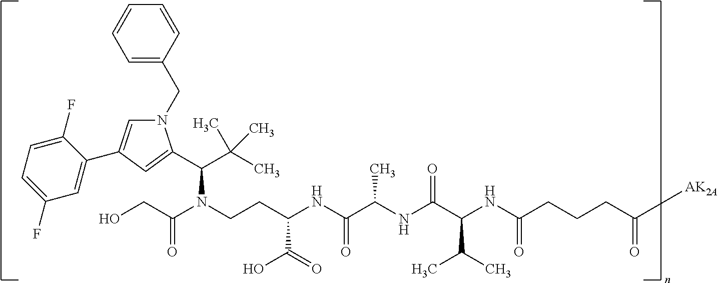

Here, 2.5 mg of anti-TWEAKR AK_{1A} (ITEM-4) in PBS (c=10 mg/ml) were used for coupling with Intermediate F294. First, 4 eq of Intermediate F294 dissolved in 25 μl of DMSO were added, and after 1 h of stirring at RT the same amount was added again and the reaction was stirred at RT for a further hour. The reaction was subsequently diluted to 2.5 ml with PBS buffer (pH 7.2), purified on a Sephadex column, then concentrated by ultracentrifugation and rediluted with PBS.

Protein concentration: 1.52 mg/ml

Drug/mAb ratio: 4.0

### Example 296N

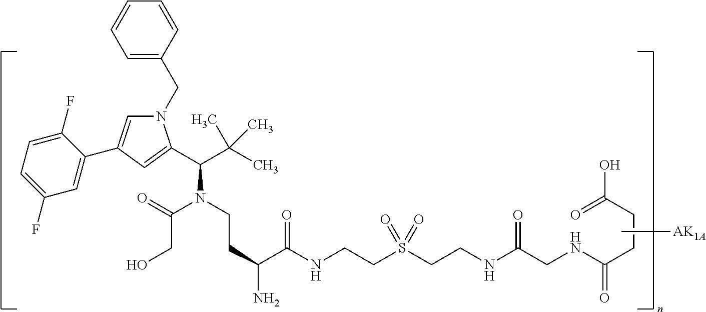

Under argon, a solution of 0.014 mg of TCEP in 25 μl of PBS buffer was added to 2.5 mg of anti-TWEAKR AK_{1A} (ITEM-4) in 250 μl of PBS (c=10 mg/ml), and the mixture was stirred at RT for 30 min. 0.105 mg (0.12 μmol) of Intermediate F296 dissolved in 25 μl of DMSO were then added. After a further 90 min of stirring at RT, the mixture was made up to 2.5 ml with PBS buffer pH 8 and passed through a PD 10 column (Sephadex® G-25, GE Healthcare) equilibrated with PBS buffer pH 8, eluted with PBS buffer pH 8 and then stirred under argon at RT overnight. The eluate was then concentrated by ultracentrifugation and rediluted with PBS buffer (pH 7.2). The ADC batch obtained was characterized as follows:

Protein concentration: 0.74 mg/ml

Drug/mAb ratio: 1.4

### Example 296O

Here, 5 mg of anti-TWEAKR AK_{1C} (TPP-7006) in 500 μl of PBS were used for coupling with intermediate F296 at pH 7.2 (c=10 mg/ml). The reduction time of the antibody in the presence of 0.029 mg of TCEP was 30 min. 0.21 mg (0.23 μmol) of F296 in 50 μl of DMSO were then added and the mixture was stirred at RT for a further 90 min. The mixture was then made up to 2.5 ml with PBS buffer (pH 8) and applied to a PD-10 column (Sephadex® G-25, GE Healthcare) equilibrated with PBS buffer pH 8, was eluted with PBS buffer pH 8 and was then stirred under argon at RT overnight. The eluate was then concentrated by ultracentrifugation and rediluted with PBS buffer (pH 7.2). The ADC batch obtained was characterized as follows:

Protein concentration: 1.74 mg/ml

Drug/mAb ratio: 3.0

### Example 296P

Here, 5 mg of anti-TWEAKR AK_{1B} (TPP-7005) in 500 μl of PBS were used for coupling with intermediate F296 at pH 7.2 (c=10 mg/ml). The reduction time of the antibody in the presence of 0.029 mg of TCEP was 30 min. 0.21 mg (0.23 μmol) of F296 in 50 μl of DMSO were then added and the mixture was stirred at RT for a further 90 min. The mixture was then made up to 2.5 ml with PBS buffer (pH 8) and applied to a PD-10 column (Sephadex® G-25, GE Healthcare) equilibrated with PBS buffer pH 8, was eluted with PBS buffer pH 8 and was then stirred under argon at RT overnight. The eluate was then concentrated by ultracentrifugation and rediluted with PBS buffer (pH 7.2). The ADC batch obtained was characterized as follows:

Protein concentration: 1.92 mg/ml

Drug/mAb ratio: 2.6

### Example 297N

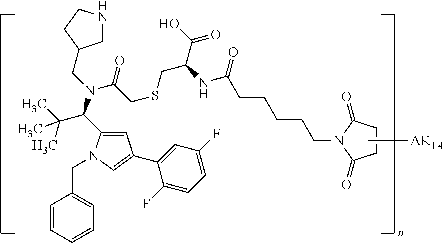

Here, 2.5 mg of anti-TWEAKR AK_{1A} (ITEM-4) in 208 μl of PBS at pH 7.2 (c=12.0 mg/ml) were used for coupling with Intermediate F297. The reduction time of the antibody in the presence of 0.014 mg of TCEP was 30 min. After addition of 0.12 mg (0.13 μmol) of F297 in 25 μl of DMSO, the reaction was then stirred at RT for 90 minutes and subsequently purified on Sephadex. The eluate was finally concentrated by ultracentrifugation and rediluted with PBS.

Protein concentration: 0.79 mg/ml

Drug/mAb ratio: 1.6

### Example 322Q

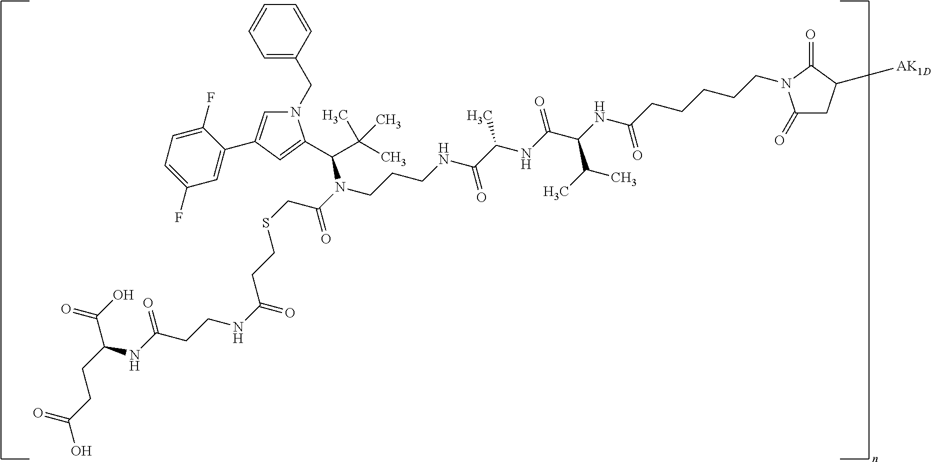

Here, 5 mg of anti-TWEAKR AK_{1D} (TPP-7007) in 450 μl of PBS were used for coupling with intermediate F322 at pH 7.2 (c=11.1 mg/ml). The reduction time of the antibody in the presence of 0.029 mg of TCEP was 30 min. After addition of 0.262 mg (0.23 μmol) of F322 in 50 μl of DMSO, the mixture was then stirred at RT for 90 min and subsequently purified on Sephadex. The mixture was then concentrated by ultracentrifugation and rediluted with PBS.

Protein concentration: 2.14 mg/ml

Drug/mAb ratio: 4.2

### Example 325P

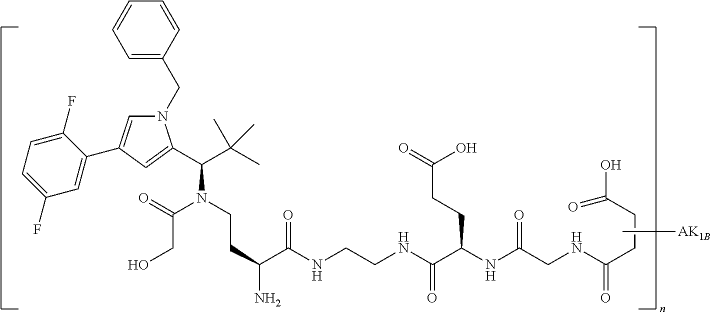

Under argon, a solution of 0.029 mg of TCEP in 50 μl of PBS buffer was added to 5 mg of anti-TWEAKR AK_{1B} (TPP-7005) in 0.450 ml of PBS (c=11.1 mg/ml). The mixture was stirred at RT for 30 min, and 0.22 mg (0.00023 mmol of Intermediate F325, dissolved in 50 μl of DMSO, was then added. After a further 90 min of stirring at RT, the mixture was made up to 2.5 ml with PBS buffer pH 8 and applied to a PD-10 column (Sephadex® G-25, GE Healthcare) equilibrated with PBS buffer pH 8, was eluted with PBS buffer pH 8 and was then stirred under argon at RT overnight. The eluate was then concentrated by ultracentrifugation and rediluted with PBS buffer (pH 7.2). The ADC batch obtained was characterized as follows:

Protein concentration: 2.09 mg/ml

Drug/mAb ratio: 3.3

### Example 326P

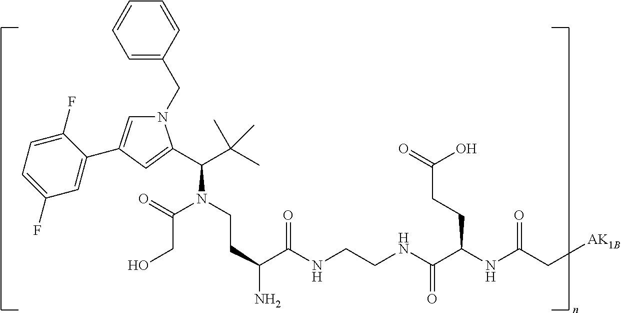

Under argon, a solution of 0.029 mg of TCEP in 50 μl of PBS buffer was added to 5 mg of anti-TWEAKR AK_{1B} (TPP-7005) in 0.450 ml of PBS (c=11.1 mg/ml). The mixture was stirred at RT for 30 min, and 0.22 mg (0.00023 mmol) of Intermediate F326, dissolved in 50 μl of DMSO, was then added. The mixture was then stirred under argon at RT overnight, made up to 2.5 ml with PBS buffer (pH 7.2) and passed over a PD-10 column (Sephadex® G-25, GE Healthcare) equilibrated with PBS buffer pH 7.2. The eluate was then concentrated by ultracentrifugation and rediluted with PBS buffer (pH 7.2). The ADC batch obtained was characterized as follows:

Protein concentration: 1.88 mg/ml

Drug/mAb ratio: 4.4

### C: Assessment of Biological Efficacy

The biological activity of the compounds according to the invention can be shown in the assays described below:

a. C-1a Determination of the Cytotoxic Effects of the ADCs Directed Against TWEAKR

The analysis of the cytotoxic effects of the ITEM-4 ADCs was carried out with various cell lines:

NCI-H₂₉₂: human mucoepidermoid lung carcinoma cells, ATCC-CRL-1848, standard medium: RPMI 1640 (Biochrom; #FG1215, stab, glutamine)+10% ECS (Sigma; #F2442), TWEAKR-positive, EGFR-positive. LoVo human colorectal cancer cells, ATCC No. CCL-229, cultivation for MTT assay: standard medium: Kaighn's+L-glutamine (Invitrogen 21127)+10% heat inactivated ECS (from Gibco, No. 10500-064). Cultivation for the CTG assay: RPMI 1640 (Biochrom; #FG1215, stab, glutamine)+10% ECS (Sigma #F2442). TWEAKR-positive.

BxPC3: human pancreas carcinoma cells, ATCC-CRL-1687, standard medium: RPMI 1640 (Biochrom; #FG1215, stab, glutamine)+10% ECS (Sigma #F2442), TWEAKR-positive.

KPL4: human breast cancer cell line, Bayer Pharma AG (identity checked and confirmed on 19.7.2012 at DSMZ), standard medium: RPMI 1640 (from Gibco; #21875-059, stab. L-glutamine)+10% heat inactivated ECS (from Gibco, No. 10500-064); HER2-positive.

A498: human kidney cancer cells, ATCC No. HTB-44, standard medium: MEM with Earle's salts+Glutamax I (Invitrogen 41090)+10% heat inactivated ECS (from Gibco, No. 10500-064), TWEAKR-positive.

786-O: human kidney cancer cells, ATCC No. CRL-1932, standard medium: RPMI 1640+Glutamax I (Invitrogen 61870)+10% heat inactivated ECS (from Gibco, No. 10500-064), TWEAKR-positive.

SK-HEP-1: human liver cancer cell line, ATCC No. HTB-52, standard medium: MEM with Earle's salts+Glutamax I (Invitrogen 41090)+10% heat inactivated ECS (from Gibco, No. 10500-064); TWEAKR-positive.

The cells were cultivated by the standard method as stated by the American Tissue Culture Collection (ATCC) for the cell lines in question.

### MTT Assay

The cells were cultivated according to the standard method using the growth media listed under C-1. The test is carried out by detaching the cells with a solution of Accutase in PBS (Biochrom AG #L2143), pelleting, resuspending in culture medium, counting and sowing into a 96-well culture plate with white bottom (Costar #3610) (NCI H292: 2500 cells/well, KPL-4: 1200 cells/well, SK-HEP-1: 1500 cells/well in a total volume of 100 μl). The cells were then incubated in an incubator at 37° C. and 5% carbon dioxide. After 48 h, the medium was replaced. The metabolites in 10 μl of culture medium in concentrations from 10⁻⁵M to 10⁻¹³M were then pipetted to the cells (in triplicate), and the assay was then incubated in an incubator at 37° C. and 5% carbon dioxide. After 96 h, the cell proliferation was detected using the MTT assay (ATCC, Manassas, Va., USA, catalogue No. 30-1010K). To this end, the MTT reagent was incubated with the cells for 4 h, followed by lysis of the cells overnight by addition of the detergent. The dye formed was detected at 570 nm (Infinite M1000 pro, Tec an). The measured data were used to calculate the IC₅₀ of the growth inhibition using the DRC (dose response curve). The proliferation of cells which were not treated with test substance but were otherwise identically treated was defined as the 100% figure.

### CTG Assay

The cells were cultivated according to the standard method using the growth media listed under C-1. A test was carried out by detaching the cells with a solution of trypsin (0.05%) and EDTA (0.02%) in PBS (Biochrom AG #L2143), pelleting, resuspending in culture medium, counting and sowing into a 96-well culture plate with white bottom (Costar #3610) (in 75 μl/well, the following cell numbers per well: NCI-H292: 2500 cells/well, BxPC3 2500 cells/well) and incubating in an incubator at 37° C. and 5% carbon dioxide. After 24 h, the antibody drug conjugates in 25 μl of culture medium (four-fold concentrated) were added to the cells such that final concentrations of antibody drug conjugates of 3×10⁻⁷ M to 3×10⁻¹¹ M on the cells were reached (in triplicate). The cells were then incubated in an incubator at 37° C. and 5% carbon dioxide. In a parallel plate, the cell vitality was determined at the start of the drug treatment (day 0) using the Cell Titer Glow (CTG) Luminescent Cell Viability Assay (Promega #G7573 and #G7571). To this end, 100 μl of the substrate were added per cell batch, the plates were then covered with aluminium foil, shaken on the plate shaker at 180 rpm for 2 minutes, allowed to stand on the laboratory bench for 8 minutes and then measured using a luminometer (Victor X2, Perkin Elmer). The substrate detects the ATP content in the living cells, generating a luminescence signal the height of which is directly proportional to the vitality of the cells. After 72 h of incubation with the antibody drug conjugates, in these cells, too, the vitality was determined using the Cell Titer Glow Luminescent Cell Viability Assay as described above. From the measured data, the IC₅₀ of the growth inhibition in comparison to day 0 was calculated using the DRC (dose response curve) analysis spreadsheets with a 4-parameter fit. The DRC analysis spreadsheet is a Biobook Spreadsheet developed by Bayer Pharma AG and Bayer Business Services on the IDBS E-WorkBook Suite platform (IDBS: ID Business Solutions Ltd., Guildford, UK).

Tables 1a and 1b below list the IC₅₀ values of representative working examples for the anti-TWEAKR antibodies:

TABLE 1a

| | BxPC3 | NCI-H292 | LoVo |
|---|---|---|---|
| | IC₅₀ [M] | IC₅₀ [M] | IC₅₀ [M] |
| Example | CTG | CTG | CTG |
| 194n | 1.16E−09 | 1.62E−09 | 1.57E−08 |
| 208n | 1.26E−09 | 4.46E−10 | 1.12E−10 |
| 257n | 1.37E−09 | 6.73E−10 | 1.47E−10 |
| 260n | 6.93E−09 | 3.27E−09 | 6.00E−07 |
| 274n | 6.00E−07 | 3.49E−08 | 6.00E−07 |
| 275n | 1.72E−08 | 1.07E−08 | 2.38E−10 |
| 281n | 6.00E−07 | 6.00E−07 | 8.71E−09 |
| 284n | 1.10E−09 | 5.59E−10 | 8.80E−11 |
| 294n | 2.13E−08 | 6.00E−07 | 6.00E−07 |
| 296n | 2.78E−09 | 1.36E−09 | 2.34E−10 |
| 297n | 3.10E−09 | 1.48E−09 | 2.30E−07 |

TABLE 1b

| | NCI-H292 | SK-Hep-1 |
|---|---|---|
| | IC₅₀ [M] | IC₅₀ [M] |
| Example | MTT | MTT |
| 208o | 7.25E−11 | 3.05E−11 |
| 208p | 1.25E−10 | 2.07E−11 |
| 208q | 1.14E−10 | 2.17E−11 |
| 240o | 5.66E−11 | 1.36E−11 |
| 257p | 2.43E−11 | 1.13E−11 |
| 281p | 1.87E−11 | 1.10E−11 |
| 281q | 4.31E−11 | 1.59E−11 |
| 284o | 2.26E−10 | 3.80E−11 |
| 284p | 2.16E−10 | 2.72E−11 |
| 296o | 2.80E−10 | 5.61E−11 |
| 296p | 2.58E−10 | 7.61E−11 |
| 322q | 2.62E−11 | 1.32E−11 |
| 325p | 2.47E−11 | 2.30E−11 |
| 326p | 8.81E−11 | 2.60E−11 |

The activity data reported relate to the working examples described in the present experimental section, with the drug/mAB ratios indicated. The values may possibly deviate for different drug/mAB ratios. The IC₅₀ values are means of several independent experiments or individual values. The action of the anti-TWEAKR antibody drug conjugates was selective versus the respective isotype control comprising the respective linker and toxophor.

### C-1b Determination of the Inhibition of the Kinesin Spindle Protein KSP/Eg5 by Selected Examples

The motor domain of the human kinesin spindle protein KSP/Eg5 (tebu-bio/Cytoskeleton Inc, No. 027EG01-XL) was incubated in a concentration of 10 nM with microtubuli (bovine or porcine, tebu-bio/Cytoskeleton Inc) stabilized with 50 μg/ml taxol (Sigma No. T7191-5MG) for 5 min at RT in 15 mM PIPES, pH 6.8 (5 mM MgCl₂ and 10 mM DTT, Sigma). The freshly prepared mixture was aliquoted into a 384 MTP (Corning). The inhibitors to be examined at concentrations of 1.0×10-6 M to 1.0×10-13 M and ATP (final concentration 500 μM, Sigma) were then added. Incubation was at RT for 2 h. ATPase activity was detected by detecting the inorganic phosphate formed using malachite green (Biomol). After addition of the reagent, the assay was incubated at RT for 50 min prior to detection of the absorption at a wavelength of 620 nm. The positive controls used were monastrol (Sigma, M8515-1mg) and ispinesib (AdooQ Bioscience A10486). The individual data of the dose-activity curve are eight-fold determinations. The IC₅₀ values are means of two independent experiments. The 100% control was the sample which had not been treated with inhibitors.

Table 2 below summarizes the IC₅₀ values of representative working examples from the assay described and the corresponding cytotoxicity data (MTT assay).

TABLE 2

| | | NCI-H292 | KPL4 |
|---|---|---|---|
| | KSP assay | IC₅₀ [M] | IC₅₀ [M] |
| Examples | IC₅₀ [M] | MTT assay | MTT assay |
| M1 | 2.01E−09 | 5.00E−07 | 5.00E−07 |
| M2 | 2.45E−09 | 2.04E−07 | 1.63E−07 |
| M3 | 1.52E−09 | 3.21E−08 | 9.00E−08 |
| M4 | 2.71E−10 | 4.43E−08 | 1.76E−07 |
| M5 | 4.57E−10 | 7.94E−08 | 2.22E−07 |
| M6 | 1.78E−09 | 4.63E−08 | 1.93E−07 |
| M7 | 6.21E−10 | 2.22E−08 | 9.25E−08 |
| M9 | 1.07E−09 | 7.74E−10 | 2.57E−10 |
| M10 | 4.70E−10 | 3.03E−07 | 2.26E−07 |
| M11 | 1.11E−09 | 4.32E−11 | |
| M12 | 4.46E−10 | 3.3E−08 | |
| M13 | 1.50E−09 | 1.52E−07 | 1.69E−07 |
| M14 | 2.16E−09 | 1.74E−07 | 1.82E−07 |
| M15 | 9.64E−10 | 1.33E−07 | 1.69E−07 |
| M16 | 1.48E−09 | 1.43E−07 | 1.95E−07 |
| M17 | 4.17E−09 | 7.35E−09 | |
| M18 | 5.17E−09 | 3.55E−08 | |
| M19 | 2.58E−09 | 1.21E−07 | |
| M20 | 1.50E−09 | 1.49E−07 | 2.13E−07 |
| M21 | 2.31E−09 | | |
| M22 | 8.27E−10 | 2.89E−08 | 1.82E−07 |
| M23 | 1.26E−09 | 5.00E−07 | 5.00E−07 |
| M24 | 2.90E−09 | 1.67E−07 | 5.00E−07 |
| M25 | 2.91E−09 | 5.00E−07 | 5.00E−07 |
| M26 | 9.441E−10 | 6.38E−08 | |
| M27 | 2.03E−09 | 2.76E−07 | |

The activity data reported relate to the working examples described in the present experimental section.

### C-2 Internalisation Assay

Internalisation is a key process which enables specific and efficient provision of the cytotoxic payload in antigen-expressing cancer cells via antibody drug conjugates (ADC). This process is monitored via fluorescent labelling of specific anti-TWEAKR antibodies and an isotype control antibody. First, the fluorescent dye was conjugated to lysines of the antibody. Conjugation was carried out using a two-fold molar excess of CypHer 5E mono NHS ester (Batch 357392, GE Healthcare) at pH 8.3. After the coupling, the reaction mixture was purified by gel chromatography (Zeba Spin Desalting Columns, 40K, Thermo Scientific, No. 87768; elution buffer: DULBECCO S PBS, Sigma-Aldrich, No. D8537), to eliminate excess dye and to adjust the pH. The protein solution was concentrated using VIVASPIN 500 columns (Sartorius stedim biotec). The dye load of the antibody was determined by means of spectrophotometric analysis (NanoDrop) and subsequent calculation (D: P=A_{dye}εₚᵣₒₜₑᵢₙ: (A₂₈₀−0.16A_{dye})ε_{dye}).

The dye load of the anti-TWEAKR antibody examined here and the isotype control were of a comparable order. In cell binding assays, it was confirmed that the conjugation did not lead to a change in the affinity of the antibody.

The labelled antibodies were used in the internalization assays.

Prior to the start of this treatment, cells 2×10⁴/well) in 100 μl of medium were sown in a 96-MTP (fat, black, clear bottom No 4308776, from Applied Biosystems). After 18 h of incubation at 37° C./5% CO₂, the medium was replaced and labelled anti-TWEAKR antibodies were added in various concentrations (10, 5, 2.5, 1, 0.1 μg/ml). The same treatment scheme was used for the labelled isotype control (negative control). The chosen incubation times were 0 h, 0.25 h, 0.5 h, 1 h, 1.5 h 2 h, 3 h, 6 h and 24 h. Fluorescence measurement was carried out using the InCellAnalyzer 1000 (from GE Healthcare). Kinetic evaluation was carried out via measurement of the parameters granule counts/cell and total granule intensity/cell.

After binding to TWEAKR, anti-TWEAKR antibodies were examined for their internalization ability. To this end, human tumour cells having various TWEAKR expression levels were chosen (e.g. NCI H292, 786-0, A498). Target-mediated specific internalization with the anti-TWEAKR antibodies were observable in the different cell lines, whereas the isotype control showed no internalization.

### C-3 In Vitro Tests for Determining Cell Permeability

The cell permeability of a substance can be investigated by means of in vitro testing in a flux assay using Caco-2 cells [M. D. Troutman and D. R. Thakker, Pharm. Res. 20 (8), 1210-1224 (2003)]. For this purpose, the cells were cultured for 15-16 days on 24-well filter plates. For the determination of permeation, the respective test substance was applied in a HEPES buffer to the cells either apically (A) or has ally (B) and incubated for 2 hours. After 0 hours and after 2 hours, samples were taken from the cis and trans compartments. The samples were separated by HPLC (Agilent 1200, Böblingen, Germany) using reverse phase columns. The HPLC system was coupled via a Turbo Ion Spray Interface to a Triple Quadropol mass spectrometer API 4000 (AB SCIEX Deutschland GmbH, Darmstadt, Germany). The permeability was evaluated on the basis of a Pₐₚₚ value, which was calculated using the formula published by Schwab et al. [D. Schwab et al., J. Med. Chem. 46, 1716-1725 (2003)]. A substance was classified as actively transported when the ratio of Pₐₚₚ (B-A) to Pₐₚₚ (A-B) (efflux ratio) was >2 or <0.5.

Of critical importance for toxophores which are released intracellularly is the permeability from B to A [Pₐₚₚ (B-A)] and the ratio of Pₐₚₚ (B-A) to Pₐₚₚ (A-B) (efflux ratio): the lower this permeability, the slower the active and passive transport processes of the substance through the monolayer of Caco-2 cells. If additionally the efflux ratio does not indicate any active transport, the substance may, following intracellular release, remain longer in the cell. Hence, there is also more time available for interaction with the biochemical target (in this case: kinesin spindle protein, KSP/Eg5).

Table 3 below sets out permeability data for representative working examples from this assay:

TABLE 3

| Working | Pₐₚₚ (B-A) | |
|---|---|---|
| Example | [nm/s] | Efflux ratio |
| M1 | 7.8 | 4 |
| M2 | 4.8 | 6.4 |
| M3 | 1.4 | 1.3 |
| M4 | 21.3 | 18.7 |
| M5 | 20.3 | 26.5 |
| M6 | 1.7 | 0.7 |
| M7 | 5.6 | 2.2 |
| M9 | 213 | 16 |
| M11 | 24.3 | 27.7 |
| M12 | 3.3 | 1.8 |
| M13 | 7.1 | 3.6 |
| M14 | 12.7 | 6.6 |
| M15 | 6.4 | 4.4 |
| M16 | 9.0 | 7.0 |
| M17 | 93.6 | 81.5 |
| M18 | 1.6 | 2.9 |
| M19 | 1.9 | 2.9 |
| M21 | 0.5 | 1.5 |
| M22 | 0.9 | 0.9 |
| M23 | 2.8 | 2.0 |
| M24 | 3.9 | 1.0 |
| M25 | 8.1 | 3.6 |
| M26 | 13.0 | 9.6 |
| M27 | 13.2 | 11.9 |

### C-4 In Vitro Tests for Determining the Substrate Properties for P-Glycoprotein (P-gp)

Many tumour cells express transporter proteins for drugs, and this frequently accompanies the development of resistance towards cytostatics. Substances which are not substrates of such transporter proteins, such as P-glycoprotein (P-gp) or BCRP, for example, could therefore exhibit an improved activity profile.

The substrate properties of a substance for P-gp (ABCB1) were determined by means of a flux assay using LLC-PK1 cells which overexpress P-gp (L-MDR1 cells) [A. H. Schinkel et al., J. Clin. Invest. 96, 1698-1705 (1995)]. For this purpose, the LLC-PK1 cells or L-MDR1 cells were cultured on 96-well filter plates for 3-4 days. For determination of the permeation, the respective test substance, alone or in the presence of an inhibitor (such as ivermectin or verapamil, for example), was applied in a HEPES buffer to the cells either apically (A) or has ally (B) and incubated for 2 hours. After 0 hours and after 2 hours, samples were taken from the cis and trans compartments. The samples were separated by HPLC using reverse phase columns. The HPLC system was coupled via a Turbo Ion Spray Interface to a Triple Quadropol mass spectrometer API 3000 (Applied Biosystems Applera, Darmstadt, Germany). The permeability was evaluated on the basis of a Pₐₚₚ value, which was calculated using the formula published by Schwab et al. [D. Schwab et al., J. Med. Chem. 46, 1716-1725 (2003)]. A substance was classified as P-gp substrate when the efflux ratio of Pₐₚₚ (B-A) to Pₐₚₚ (A-B) was >2.

As further criteria for the evaluation of the P-gp substrate properties, the efflux ratios in L-MDR1 and LLC-PK1 cells or the efflux ratio in the presence or absence of an inhibitor may be compared. If these values differ by a factor of more than 2, the substance in question is a P-gp substrate.

### C-5 Pharmacokinetics

After i.v. administration of 3-30 mg/kg of different ADCs, the plasma and tumour concentrations of the antibody parts of the ADCs can be measured by means of ELISA (see section: Analysis for quantification of antibodies) and the pharmacokinetic parameters such as clearance (CL), area under the curve (AUC) and half-times (t_{1/2}) can be calculated. In analogy thereto, the potentially occurring metabolite concentrations of the ADCs in plasma, tumour and tissue can be measured.

After administration of 5 mg/kg i.v. of the ADC of Example 208o in male rats, the following parameters for the ADC were able to be determined:

| Parameter | Example 208o |
|---|---|
| AUCnorm (kg × h/L) | 2313 |
| AUC (mg × h/l) | 11563 |
| Cmax, norm (kg/l) | 26 |
| Cmax (mg/l) | 131 |
| CL (ml/h/kg) | 0.43 |
| Vss (l/kg) | 0.098 |
| MRT (h) | 226 |
| T ½ (h) | 171 |

### Analysis for Quantification of the Antibodies Used

The antibody part of the ADCs was determined using a ligand binding assay (ELISA) as total IgG concentration in plasma samples and tumour lysates. Here, the sandwich ELISA format was used. This ELISA had been qualified and validated for the determination in plasma and tumour samples. The ELISA plates were coated with anti-human goat IgG Fc antibodies. After incubation with the sample, the plates were washed and incubated with a detector conjugate of simian anti-human IgG(H+L) antibody and horseradish peroxidase (HRP). After a further washing step, the HRP substrate was added to OPD and the colour development was monitored via absorption at 490 nm. Standard samples having a known IgG concentration were fitted using a 4-parameter equation. Within the lower (LLOQ) and upper (ULOQ) quantification limits, the unknown concentrations were determined by interpolation.

C5a: Identification of the ADC Metabolites after Internalisation In Vitro

### Description of the Method:

Internalisation studies with immunoconjugates were carried out to analyse metabolites formed intracellularly. To this end, human lung tumour cells NCI H292 (3×10⁵/well) were sown in 6-well plates and incubated overnight (37° C., 5% CO₂). The cells were treated with 10 μg/ml (66 nM) of the ADC to be examined. Internalisation was carried out at 37° C. and 5% CO₂. At various time points (0, 4, 24, 48, 72 h), cell samples were taken for further analysis. First, the supernatants (about 5 ml) were harvested and, after centrifugation (2 min, RT, 1000 rpm Heraeus Variofuge 3.0R), stored at −80° C. The cells were washed with PBS and detached with Accutase, and the cell number was determined. After another washing, a defined number of cells (2×10⁵) was treated with 100 ml of lysis buffer (Mammalian Cell Lysis Kit (Sigma MCL1) and incubated with continuous shaking (Thermomixer, 15 min, 4° C., 650 rpm) in Protein LoBind tubes (Eppendorf Cat. No. 0030 108.116). After the incubation, the lysate was centrifuged (10 min, 4° C., 12000 g, Eppendorf 5415R) and the supernatant was harvested. The supernatant obtained was stored at −80° C. All samples were then analysed as follows.

Measurement of the compounds in the culture supernatant or cell lysate was carried out after precipitation of the proteins with methanol or acetonitrile by high-pressure liquid chromatography (HPLC) coupled to a triple-quadrupole mass spectrometer (MS).

For work-up of 50 μl of culture supernatant/cell lysate, 150 μl of precipitation reagent (generally acetonitrile) were added and the mixture was shaken for 10 seconds. The precipitation reagent contained an internal standard (ISTD) in a suitable concentration (generally in the range of 20-100 ng/ml). After 3 minutes of centrifugation at 16000 g, the supernatant was transferred into an autosampler vial, made up with 500 μl of a buffer suitable for the mobile phase and shaken again. The two matrix samples were then measured using the HPLC-coupled triple-quadrupol mass spectrometer API6500 from AB SCIEX Deutschland GmbH.

For calibration, concentrations of 0.5-2000 μg/1 were added to plasma samples. The detection limit (LOQ) was about 2 μg/1. The linear range extended from 2 to 1000 μg/1.

For calibration of the tumour samples, concentrations of 0.5-200 μg/1 were added to the supernatant of untreated tumours. The detection limit was 4 μg/1. The linear range extended from 4 to 200 μg/1.

Quality controls for testing validity contained 5 and 50 μg/1.

### C5b: Identification of the ADC Metabolites In Vivo

As reference example (R10k), an ADC with the agonistic antibody TPP-2658 was produced:

### REFERENCE EXAMPLE R10K

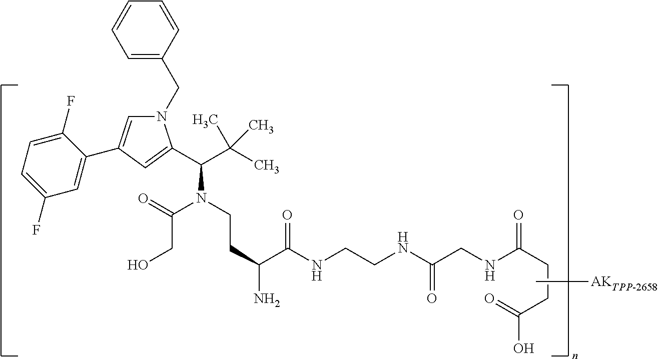

Under argon, a solution of 0.86 mg of TCEP in 2 ml of PBS buffer was added to 150 mg of anti-TWEAKR antibody TPP-2658 in 10.5 ml of PBS (c=14.28 mg/ml). The antibody TPP-2658 and its preparation are described in detail in WO 2015/189143 A1.

The mixture was stirred at RT for 30 min, and 6.63 mg (0.008 mmol) of Intermediate F104, dissolved in 1250 μl of DMSO, were then added. After a further 90 min of stirring at RT, the mixture was diluted with 1250 μl of PBS buffer which had been adjusted to pH 8 beforehand. This solution was then applied to PD-10 columns (Sephadex® G-25, GE Healthcare) equilibrated with PBS buffer pH 8 and was eluted with PBS buffer pH 8. The eluate was diluted to a total volume of 22.5 ml with PBS buffer pH 8. This solution was stirred under argon at RT overnight and then, once more using PD-10 columns, rebuffered to pH 7.2. The mixture was then concentrated by ultracentrifugation, rediluted with PBS buffer (pH 7.2) and concentrated again. The ADC batch obtained was characterized as follows:

Protein concentration: 14.06 mg/ml

Drug/mAb ratio: 3.4

### Analysis for Quantification of any Metabolites Occurring

Measurement of the compounds in plasma, tumour and tissue was carried out after precipitation of the proteins with methanol or acetonitrile by high-pressure liquid chromatography (HPLC) coupled to a triple-quadrupole mass spectrometer (MS).

For work-up of 50 μl of plasma, 250 μl of precipitation reagent (generally acetonitrile) were added and the mixture was shaken for 10 seconds. The precipitation reagent contained an internal standard (ISTD) in a suitable concentration (generally in the range of 20-100 ng/ml). After 3 minutes of centrifugation at 16000 g, the supernatant was transferred into an autosampler vial, made up with 500 μl of a buffer suitable for the mobile phase and shaken again.

During the work-up of a tumour or tissue, the tumour or tissue was treated with 3 times the amount of extraction buffer. The extraction buffer contained 50 ml of Tissue Protein Extraction Reagent (Pierce, Rockford, Ill.), two pellets of Complete-Protease-Inhibitor-Cocktail (Roche Diagnostics GmbH, Mannheim, Germany) and phenylmethylsulphonyl fluoride (Sigma, St. Louis, Mo.) in a final concentration of 1 mM. The sample was homogenized twice for 20 minutes in a Tissuelyser II (Qiagen), at maximum stroke number. 50 μl of the homogenate were transferred into an autosampler vial and made up with 150 μl of methanol including ISTD. After 3 minutes of centrifugation at 16000 g, 10 μl of the supernatant were made up with 180 μl of a buffer suitable for the mobile phase and shaken again. The tumour or tissue sample was then ready for measuring.

The matrix samples were then measured using the HPLC-coupled triple-quadrupol mass spectrometer API6500 from AB SCIEX Deutschland GmbH.

For calibration, concentrations of 0.5-2000 μg/1 were added to plasma samples. The detection limit (LOQ) was about 2 μg/1. The linear range extended from 2 to 1000 μg/1.

For calibration of the tumour and tissue samples, concentrations of 0.5-200 μg/1 were added to the supernatant of untreated tumours or tissue. The detection limit was between 3 and 6 μg/1. The linear range extended from 3 to 200 μg/1.

Quality controls for testing validity contained 5 and 50 μg/1, in plasma additionally 500 μg/1.

TABLE
Catabolite concentrations in NCI H292 Xenograft mouse
tumour, liver and kidney 24 h after administration of 10 mg/kg
of the ADC from Example 208o or of 10 mg/kg of the ADC from
Reference example R10k (measured catabolite in both cases: M26).

| | | M26 | M26 |
|---|---|---|---|
| | | Mean [μg/L] | SD [μg/L] |
| Tumour | R10k | 180.3 | 4.3 |
| | Example 208o | 214.1 | 29.1 |
| Liver | R10k | 90.7 | 18.2 |
| | Example 208o | 23.1 | 7.2 |
| Kidney | R10k | 81.6 | 12.6 |
| | Example 208o | 13.0 | 2.2 |

After administration of the ADC Example 208o according to the invention with a moderately agonistic antibody, in the tumour, the concentrations of active catabolite M26 measured were comparable to those after administration of the ADC R10k with the same payload and a very agonistic antibody (the agonistic activity of the antibody TPP-2658 is described in detail in WO 2015/189143 A1). In contrast, the measured concentrations of the active metabolites in liver and kidney were markedly lower after administration of the ADC from Example 208o compared to after the administration of the reference ADC R10k. There is a markedly more selective release of the drug at the target tissue (tumour) compared to other organs when a non-agonistically or only moderately agonistically acting anti-TWEAKR antibody is used in the conjugate.

### C-6 Efficacy Test In Vivo

The efficacy of the conjugates according to the invention can be tested in vivo using, for example, xenograft models. The person skilled in the art is familiar with methods from the prior art which can be used to test the efficacy of the compounds according to the invention (see, for example, WO 2005/081711; Poison et al., Cancer Res. 2009 Mar. 15; 69(6):2358-64). To this end, for example, a tumour cell line expressing the target molecule of the binder is implanted into rodents (e.g. mice). Subsequently, a conjugate according to the invention, an isotype antibody control conjugate or a control antibody or isotonic saline is then administered to the implant animals. Administration is carried out once or more than once. After an incubation time of several days, the tumour size is determined by comparing conjugate-treated animals and the control group. The conjugate-treated animals showed a smaller tumour size.

C-6a. Growth Inhibition/Regression of Experimental Tumours in Mice

Human tumour cells expressing the antigen for the antibody drug conjugate were inoculated subcutaneously into the flank of immunosuppressed mice, for example NMRi nude or SCID mice. 1-10 million cells were detached from the cell culture, centrifuged and resuspended in medium or medium/Matrigel. The cell suspension was injected under the skin of the mouse.

Within a number of days, a tumour grew. Treatment was initiated after the tumour had established, at a tumour size of approximately 40 mm². To examine the effect on larger tumours, it is also possible to initiate the treatment only at a tumour size of 50-100 mm².

The treatment of the mouse with the ADCs was carried out via the intravenous (i.v.) route in the tail vein of the mouse. The ADC was administered in a volume of 5 ml/kg.

The treatment protocol depended on the pharmacokinetics of the antibody conjugate. As a standard, there were three successive treatments on every seventh day. For a quick assessment, it is also possible to use a protocol with a single treatment. However, the treatment may also be continued, or a second cycle of three treatment days may follow at a later stage.

As standard, 8 animals were used per treatment group. In addition to the groups which received the active substances, one group, as control group, was treated only with buffer, following the same protocol.

During the course of the experiment, the tumour area was measured regularly in two dimensions (length/width) using a caliper. The tumour area was determined as length×width. The comparison of the mean tumour area of the treatment group to that of the control group was stated as T/C area.

If all groups of the experiment were terminated at the same time at the end of the treatment, the tumours could be removed and weighed. The comparison of the mean tumour weight of the treatment group to that of the control group was stated as T/C weight.

C-6b. Efficacy in Human Tumour Xenograft Models

The tumour cells in question were inoculated subcutaneously into the flank of female NMRI-nude mice (Janvier). At a tumour size of ˜40 mm², the animals were treated intravenously with the antibody drug conjugate. Subsequent to the treatment, tumour growth was optionally monitored further.

Treatment with the anti-TWEAKR antibody drug conjugates resulted in a marked and long-lasting growth inhibition of the tumours compared to the control group and the isotype drug conjugates (the inactivity of the latter had been demonstrated in previous experiments). The T/C values, determined over the tumour area on the day with the last measured value of the control group, calculated after inoculation, are stated in Table 8.

TABLE 8

| | | | Dosage | |
|---|---|---|---|---|
| Example | Tumour model | Dose | scheme | T/C area |
| 208-p | KU-19-19 (human | 5 mg/kg | Q7dx3 | 0.26 |
| | bladder carcinoma) | | | (Day 15) |
| 208-p | NCI-H292 (human | 5 mg/kg | Q7dx3 | 0.22 |
| | lung carcinoma) | | | (Day 19) |
| 208-o | KU-19-19 (human | 5 mg/kg | Q7dx3 | 0.27 |
| | bladder carcinoma) | | | (Day 15) |
| 208-o | NCI-H292 (human | 5 mg/kg | Q7dx3 | 0.25 |
| | lung carcinoma) | | | (Day 19) |

### WORKING EXAMPLES OF ANTI-TWEAKR ANTIBODIES

### Determination of the Binding Affinity of the Antibodies by Surface Plasmon Resonance:

Surface plasmon resonance experiments for quantitative binding analysis were carried out using a Biacore T200 instrument (GE Healthcare Biacore, Inc.). Here the antibodies to be examined were fixed with the aid of an anti-human Fc antibody (“Human Antibody Capture Kit”, BR-1008-39, GE Healthcare Biacore, Inc.) amine-coupled to the sensor chip surface. Amine coupling was performed according to the manufacturer's instructions using 1-ethyl-3-(3-dimethylaminopropyl)carbodiimide hydrochloride (EDC), N-hydroxysuccinimide (NHS) and ethanolamine HCl pH 8.5 (“Amine Coupling Kit” BR-1000-50, GE Healthcare Biacore, Inc.). For the analyses, Series S sensor chips CM5 (GE Healthcare Biacore, Inc.) were used with the mobile buffer HBS-EP+ (10 mM HEPES pH 7.4, 150 mM NaCl, 3 mM EDTA, 0.05% Surfactant P20). All experimental steps were carried out at 25° C. After fixation of the anti-TWEAKR antibodies to be examined, injections of the extracellular domain of TWEAKR (Analyt, 30R-AT080, Fitzgerald) in a concentration range of from 3.9 to 500 nM were carried out, and after each antigen injection the sensor surface was regenerated with glycine HCl pH 2.0. Prior to another analyte injection, antibodies were in each case fixed as above, under identical conditions. For all measurements, an upstream flow cell only containing immobilized amine-coupled anti-human Fc antibody was used as reference cell. Evaluation of the sensorgrams obtained was, after double referencing (subtraction of the reference flow cell signal and a buffer injection), carried out by the “steady-state” affinity evaluation method implemented in the Biacore T200 evaluation software (GE Healthcare Biacore, Inc.).

TABLE 5a
Recombinant antigen used for affinity determination

| | | | Cat. No. |
|---|---|---|---|
| Nomenclature | Description | Origin | (Fitzgerald Inc) |
| TPP-2305 | hTNFRSF12amino acids | human | 30R-AT080 |
| | a28-80 | | |

TABLE 5b
Commercially available antibody

| Nomenclature | Description | Cat. No. |
|---|---|---|
| ITEM-4 | Murine IgG2b | 13-9018 (eBioscience Ref |
| | | 7016-9018 M010) |

TABLE 5c
Monovalent K_{D} value of anti-TWEAKR antibody by means
of Biacore analysis

| | ka (1/Ms) | kd (1/s) | K_{D} (nM) |
|---|---|---|---|
| ITEM-4 | 2.80E+06 | 2.00E−03 | 7.0E−10 |
| TPP-7005 | 6.8E+06 | 1.8E−03 | 2.6E−10 |
| TPP-7006 | 7.4E+06 | 2.4E−03 | 3.3E−10 |
| TPP-7007 | 5.3E+06 | 7.5E−03 | 1.4E−09 |
| TPP-7075 | 4.7E+06 | 2.8E−02 | 6.0E−09 |

### Determination of the Binding Affinity of the Antibodies to TWEAKR-Expressing Cancer Cells by Means of FACS Analysis

Binding of the anti-TWEAKR antibodies was examined by flow cytometry using different human tumour cell lines. To this end, the cells (5×10⁵ cells/well) were incubated in FACS buffer (PBS without Ca/Mg, 3% ECS, Biochrom) with 10 μg/ml primary antibody solution (start concentration) on ice for 30-45 min protected from light. A dose activity curve (1:5 dilution) was plotted. After the incubation, 200 μl of ice-cold FACS buffer were added using a pipette, and the cell suspension was centrifuged at 4° C. 400 g for 4 min. The cell pellet was washed with 300 μl of ice-cold FACS buffer and the pellet obtained was then resuspended in 100 μl FACS buffer and incubated again with secondary antibody (monoclonal anti-kappa light chains-FITC antibody, Sigma, No. SAB4700605) in a 1:10 dilution on ice for 30 min. The cells were then washed with ice-cold FACS buffer and adjusted to a cell concentration of 0.5×10⁶ cells/ml prior to flow cytometry using a Guava flow cytometer (Millipore). Propidium iodide (final concentration 1 μg/ml) was used for live staining. The results were determined as background-corrected geo mean of the fluorescence of the antibody to be examined (Tab. 6a) or the EC50 value was determined by means of dose/effect curve (Tab. 6b).

TABLE 6a
FACS analysis: Binding of the anti-TWEAKR antibody to
different cancer cell lines.

| Cell Line | Source | ITEM-4 |
|---|---|---|
| NCI-H292 | ATCC No. CRL-1848 | ++ |
| 786-O | ATCC No. CRL-1932 | +++(+) |
| A498 | ATCC No. HTB-44 | +++ |
| SK-Hep1 | ATCC No. HTB-52 | ++(+) |

(Geo Mean-Geo Mean of the secondary antibody >5: +, >50: ++, >500: +++, >5000: ++++

TABLE 6b
FACS analysis: Binding of the humanized anti-TWEAKR
antibodies to the lung carcinoma cell line NCI-H292 and
liver cancer cell SK-HEP-1

| | NCI-H292 | SK-Hep-1 |
|---|---|---|
| | EC50 [M] | EC50 [M] |
| ITEM-4 | 1.9E−09 | n.d. |
| TPP-7005 | 5.0E−10 | 5.4E−10 |
| TPP-7006 | 5.4E−10 | 8.3E−10 |
| TPP-7007 | 8.0E−10 | 9.0E−10 |
| TPP-7075 | 8.9E−09 | 1.9E−09 |

### Determination of the Agonistic/Antagonistic Activity of the Anti-TWEAKR Antibodies by Means of NFkappaB Reporter Gene Assay

NF-kappaB reporter gene assays were carried out to assess the agonistic activity of the antibodies (human IgG1). HEK293 cells were transfected transiently with an NF-kappaB reporter construct (BioCat, cat. No. LR-0051-PA) using 293fectin according to the instructions of the manufacturer. White poly lysine-coated 384-well plates (BD) were sown with transfected cells in FI 7 media (serum-free; Invitrogen) at 37° C., 5% CO₂. The next day, the cells were stimulated with purified antibodies at different concentrations for 6 h, and subsequently a luciferase assay was carried out according to standard methods. A moderate agonistic activity e.g. for ITEM-4 was measured, which showed 14% of the agonism of the natural ligand (at 200 ng/ml) TWEAK. To examine the antagonistic activities, the assay was carried out in the presence of the natural ligand Tweak (200 ng/ml). In this assay design, a potent antagonistic activity was demonstrated for ITEM-4.

TABLE 7
Agonistic and antagonistic activity of the
anti-TWEAKR antibody

| | Agonistic activity [%] of | Antagonistic activity |
|---|---|---|
| | the TWEAK-ind. agonism | IC50 [M] |
| ITEM-4 | 14 | 2.8E−10 |
| TPP-7005 | 0.1 | 2.0E−11 |
| TPP-7006 | 3 | 1.7E−10 |
| TPP-7007 | 4 | 1.9E−09 |
| TPP-7075 | 15 | 2.4E−08 |

1: Conjugate of an antibody with one or more drug molecules of the formula below: 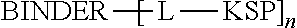
where
BINDER represents a moderately agonistically or non-agonistically acting anti-TWEAKR antibody or an antigen-binding fragment thereof,
L represents a linker,
n represents a number from 1 to 50, preferably from 1.2 to 20 and particularly preferably from 2 to 8, and
KSP represents a compound of the formula (I) below: 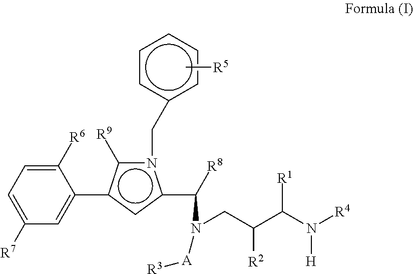
where
R¹ represents —H, -L-#1, -MOD or —(CH₂)₀₋₃Z,
where
Z represents —H, —NHY³, —OY³, —SY³, halogen, —C(═O)—NY¹Y² or —C(═O)—OY³,
Y¹ and Y² independently of one another represent —H, —NH₂, —(CH₂CH₂O)₀₋₃—(CH₂)₀₋₃Z′ (e.g. —(CH₂)₀₋₃Z′) or —CH(CH₂W)Z′,
Y³ represents —H or —(CH₂)₀₋₃Z′,
Z′ represents —H, —NH₂, —SO₃H, —COOH, —NH—C(═O)—CH₂—CH₂—CH(NH₂)COOH or —(CO—NH—CHY⁴)₁₋₃COOH;
W represents H or OH,
Y⁴ represents straight-chain or branched C₁₋₆ alkyl which is optionally substituted by —NH—C(═O)—NH₂, or represents aryl or benzyl which are optionally substituted by —NH₂;
R² represents H, -MOD, —C(═O)—CHY⁴—NHY⁵ or —(CH₂)₀₋₃Z,
where
Z represents —H, halogen, —OY³, —SY³, —NHY³, —C(═O)—NY¹Y² or —C(═O)—OY³,
Y¹ and Y² independently of one another represent —H, —NH₂ or —(CH₂)₀₋₃Z′,
Y³ represents —H or —(CH₂)₀₋₃Z′,
Z′ represents —H, —SO₃H, —NH₂ or —COOH;
Y⁴ represents straight-chain or branched C₁₋₆-alkyl which is optionally substituted by —NH—C(═O)—NH₂, or represents aryl or benzyl which are optionally substituted by —NH₂, and
Y⁵ represents —H or —C(═O)—CHY⁶—NH₂,
Y⁶ represents straight-chain or branched C₁₋₆-alkyl;
R⁴ represents —H, -L-#1, -SG_{lys}-(C═O)₀₋₁—R^{4′}, —C(═O)—CHY⁴—NHY⁵ or —(CH₂)₀₋₃Z,
where SG_{lys} represents a group which can be cleaved by lysosomal enzymes, in particular a group consisting of a di- or tripeptide,
where R^{4′} represents a C₁₋₁₀-alkyl, C₅₋₁₀-aryl or C₆₋₁₀-aralkyl, C₅₋₁₀-heteroalkyl, C₁₋₁₀-alkyl-O—C₆₋₁₀-aryl, C₅₋₁₀-heterocycloalkyl, heteroaryl, heteroarylalkyl, heteroarylalkoxy, C₁₋₁₀-alkoxy, C₆₋₁₀-aryloxy or C₆₋₁₀-aralkoxy, C₅₋₁₀-heteroaralkoxy, C₁₋₁₀-alkyl-O—C₆₋₁₀-aryloxy, C₅₋₁₀-heterocycloalkoxy group which may be mono- or polysubstituted by —NH₂,
—NH-alkyl, —N(alkyl)₂, —NH—C(═O)-alkyl,
—N(alkyl)-C(═O)-alkyl, —SO₃H, —S(═O)₂—NH₂,
—S(═O)₂—N(alkyl)₂, —COOH, —C(═O)—NH₂,
—C(═O)—N(Alkyl)₂, or —OH, —H or a group-Ox-(CH₂CH₂O)y-R^{4″},
where x represents 0 or 1,
where v represents a number from 1 to 10,
where R^{4″} represents —H, -alkyl (preferably
C1-12-alkyl), —CH2-COOH,
CH2-CH2-COOH, or
CH2-CH2-NH2;
where Z represents —H, halogen, —OY³, —SY³, NHY³, —C(═O)—NY¹Y² or —C(═O)—OY³,
where Y¹ and Y² independently of one another represent —H, —NH₂ or —(CH₂)₀₋₃Z′,
where Y³ represents —H or —(CH₂)₀₋₃Z′,
where Z′ represents —H, —SO₃H, —NH₂ or —COOH;
where Y⁴ represents straight-chain or branched C₁₋₆-alkyl which is optionally substituted by —NH—C(═O)—NH₂, or represents aryl or benzyl which are optionally substituted by —NH₂,
where Y⁵ represents —H or —C(═O)—CHY⁶—NH₂,
where Y⁶ represents straight-chain or branched C₁₋₆-alkyl;
or
R² and R⁴ together (with formation of a pyrrolidine ring) represent —CH₂—CHR¹¹— or —CHR¹¹—CH₂—,
where
R¹¹ represents —H, —NH₂, —SO₃H, —COOH, —SH, halogen (in particular F or Cl), C₁₋₄-alkyl, C₁₋₄-haloalkyl, C₁₋₄-alkoxy, hydroxyl-substituted C₁₋₄-alkyl, COO(C₁₋₄-alkyl) or —OH;
A represents —C(═O)—, —S(═O)—, —S(═O)₂—, —S(═O)₂—NH— or —C(═N—NH₂)—;
R³ represents -L-#1, -MOD or an optionally substituted alkyl, cycloalkyl, aryl, heteroaryl, heteroalkyl, heterocycloalkyl group, preferably -L-#1 or a C₁₋₁₀-alkyl, C₆₋₁₀-aryl or C₆₋₁₀-aralkyl, C₅₋₁₀-heteroalkyl, C₁₋₁₀-alkyl-O—C₆₋₁₀-aryl or C₅₋₁₀-heterocycloalkyl group which may in each case be substituted by 1-3 —OH groups, 1-3 halogen atoms, 1-3 halogenated alkyl groups (each having 1-3 halogen atoms), 1-3 O-alkyl groups, 1-3 —SH groups, 1-3 —S-alkyl groups, 1-3 —O—C(═O)-alkyl groups, 1-3 —O—C(═O)—NH-alkyl groups, 1-3 —NH—C(═O)-alkyl groups, 1-3 —NH—C(═O)—NH-alkyl groups, 1-3 —S(═O)ₙ-alkyl groups, 1-3 —S(═O)₂—NH-alkyl groups, 1-3 —NH-alkyl groups, 1-3 —N(alkyl)₂ groups, 1-3 —NH₂ groups or 1-3 —(CH₂)₀₋₃Z groups,
where
n represents 0, 1 or 2,
Z represents —H, halogen, —OY³, —SY³, —NHY³, —C(═O)—NY¹Y² or —C(═O)—OY³,
Y¹ and Y² independently of one another represent —H, —NH₂ or —(CH₂)₀₋₃Z′,
Y³ represents —H, —(CH₂)₀₋₃—CH(NH—C(═O)—CH₃)Z′, —(CH₂)₀₋₃—CH(NH₂)Z′ or —(CH₂)₀₋₃Z′,
Z′ represents —H, —SO₃H, —NH₂ or —COOH,
R⁵ represents —H, —NH₂, —NO₂, halogen (in particular F, Cl, Br), —CN, CF₃, —OCF₃, —CH₂F, —CH₂F, SH or —(CH₂)₀₋₃Z,
where
Z represents —H, —OY³, —SY³, halogen, —NHY³, —C(═O)—NY¹Y² or —CO—OY³,
Y¹ and Y² independently of one another represent —H, —NH₂ or —(CH₂)₀₋₃Z′,
Y³ represents —H or —(CH₂)₀₋₃Z′,
Z′ represents —H, —SO₃H, —NH₂ or —COOH;
R⁶ and R⁷ independently of one another represent —H, cyano, C₁₋₁₀-alkyl, fluoro-C₁₋₁₀-alkenyl, C₂₋₁₀-alkenyl, fluoro-C₂₋₁₀-alkenyl, C₂₋₁₀-alkynyl, fluoro-C₂₋₁₀-alkenyl, hydroxy, —NO₂,
NH₂, —COOH or halogen,
R⁸ represents C₁₋₁₀-alkyl, fluoro-C₁₋₁₀-alkenyl, C₂₋₁₀-alkenyl, fluoro-C₂₋₁₀-alkenyl, C₂₋₁₀-alkynyl, fluoru-C₂₋₁₀-alkenyl, C₄₋₁₀-cycloalkyl fluoro-C₄₋₁₀-cycloalkyl or —(CH₂)₀₋₂—(HZ²), which may be mono- or disubstituted, identically or differently, by —OH, —COOH or —NH2, and
where
HZ² represents a 4- to 7-membered heterocycle having up to two heteroatoms selected from N, O and S,
R⁹ represents —H, —F, —CH₃, —CF₃, —CH₂F or —CHF₂;
where
one of the substituents R¹, R³ and R⁴ represents -L-#1,
L represents the linker and #1 represents the bond to the antibody,
MOD represents —(NR¹⁰)ₙ-(G1)ₒ-G2-G3,
where
R¹⁰ represents H or C₁-C₃-alkyl;
G1 represents —NHC(═O)—, —C(═O)NH—
(where, if G1 represents —NHC(═O)—, R¹⁰ does not represent NH₂);
n represents 0 or 1;
o represents 0 or 1; and
G2 represents a straight-chain or branched hydrocarbon chain which has 1 to 10 carbon atoms and which may be interrupted once or more than once by one or more of the groups —O—, —S—, —S(═O)—, S(═O)₂, —NR^{y}—, —NR^{y}C(═O)—, C(═O)—NR^{y}—, —NR^{y}NR^{y}—, —S(═O)₂—NR^{y}NR^{y}—, —C(═O)—NR^{y}NR^{y}—
where
R^{y} represents —H, phenyl, C₁-C₁₀-alkyl, C₂-C₁₀-alkenyl or C₂-C₁₀-alkynyl, each of which may be mono- or disubstituted, identically or differently, by —NH—C(═O)—NH₂, —COOH, —OH, —NH₂, —NH—CNNH₂, sulphonamide, sulphone, sulphoxide or sulphonic acid, and/or which may be interrupted once or more than once, identically or differently, by —C(═O)—, —CR^{x}═N—O—
where
Rx represents —H, C₁-C₃-alkyl or phenyl, and
where
the hydrocarbon chain including a C₁-C₁₀-alkyl group optionally substituted on the hydrocarbon group as side chain may be substituted by —NH—C(═O)—NH₂, —COOH, —OH, —NH₂, —NH—CNNH₂, sulphonamide, sulphone, sulphoxide or sulphonic acid,
G3 represents —H or —COOH, and
where the group -MOD preferably has at least one group —COOH;
and the salts, solvates, salts of the solvates and epimers thereof.

2: Conjugate according to claim 1 where A represents —C(═O)—.

3: Conjugate according to claim 1 where R¹ represents —H, -L-#1, —COOH,
—C(═O)—NHNH₂, —(CH₂)₁₋₃NH₂, —C(═O)—NZ″(CH₂)₁₋₃ NH₂ or —C(═O)—NZ″CH₂COOH, wherein Z″ represents —H or —NH₂.

4: Conjugate according to claim 1 where R² and R⁴ represent —H or R² and R⁴ together (with formation of a pyrrolidine ring) represent —CHR¹¹—CH₂— or —CH₂—CHR¹¹—; where R¹¹ represents H, COOH, F, Me, CH₂F, OMe, CH₂OH, COO(C₁₋₄-alkyl) or OH.

5: Conjugate according to claim 1 where R³ represents -L-#1 or represents a phenyl group which may be mono- or polysubstituted by halogen, C₁₋₃-alkyl or fluoro-C₁₋₃-alkyl, or represents a C₁₋₁₀-alkyl group or fluoro-C₁₋₁₀-alkyl group which may optionally be substituted by —OY⁴, —SY⁴, —O—C(═O)—Y⁴, —O—C(═O)—NH—Y⁴, —NH—C(═O)—Y⁴, —NH—C(═O)—NH—Y⁴, —S(O)ₙ—Y⁴, —S(═O)₂—NH—Y⁴, —NH—Y⁴ or —N(Y⁴)₂,
where
n represents 0, 1 or 2,
Y⁴ represents —H, phenyl which is optionally mono- or polysubstituted by halogen, C₁₋₃-alkyl or fluoro-C₁₋₃-alkyl, or represents alkyl which may be substituted by —OH, —COOH, and/or —NH—C(═O)—C₁₋₃-alkyl.

6: Conjugate according to claim 5 where the conjugate has the formula (IIj) below: 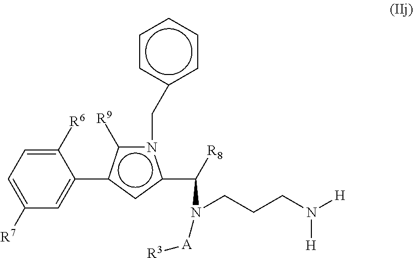
where
R³ represents-L-#1;
A represents —C(═O)—; and
R⁶, R⁷, R⁸ and R⁹ have the same meaning as in formula (I) in claim 1.

7: Conjugate according to claim 1 where the substituent R¹ represents -L-#1.

8: Conjugate according to claim 7 where the conjugate has the formula (IIk): 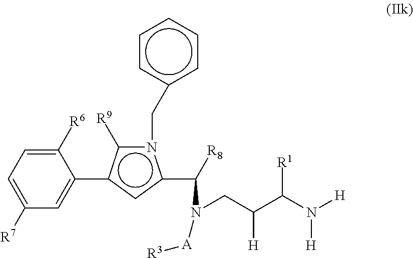
where
R¹ represents-L-#1;
A represents —C(═O)— and
R³ represents —CH₂OH—;
R⁶, R⁷, R⁸ and R⁹ have the same meaning as in formula (I) in claim 1.

9: Conjugate according to claim 1 where R⁵ represents —H or —F.

10: Conjugate according to claim 1 where R⁶ and R⁷ independently of one another represent —H, C₁₋₃-alkyl, fluoro-C₁₋₃-alkyl, C₂₋₄-alkenyl, fluoro-C₂₋₄-alkenyl, C₂₋₄-alkynyl, fluoro-C₂₋₄-alkynyl, hydroxy or halogen.

11: Conjugate according to claim 1 where R⁸ represents a branched C₁₋₅-alkyl group or cyclohexyl.

12: Conjugate according to claim 1 where R⁹ represents —H or fluorine.

13: Conjugate according to claim 1 where the linker -L- has one of the basic structures (i) to (iv) below:
(i) —(CO)ₘ—SG1-L1-L2-
(ii) —(CO)ₘ-L1-SG-L1-L2-
(iii) —(CO)ₘ-L1-L2-
(iv) —(CO)ₘ-L1-SG-L2
where m represents 0 or 1, SG and SG1 represent in vivo cleavable groups, L1 independently of one another represent organic groups not cleavable in vivo, and L2 represents a coupling group to the binder.

14: Conjugate according to claim 13 where the in vivo cleavable group SG is a 2-8 oligopeptide group, preferably a tri- or dipeptide group or a disulphide, a hydrazone, an acetal or an aminal and SG1 is a 2-8 oligopeptide group, preferably a dipeptide group.

15: Conjugate according to claim 1
where the linker L is attached to a cysteine side chain or a cysteine residue and has the formula below:
§—(C(═O)-)m-L1-L2-§§
where
m represents 0 or 1;
§ represents the bond to the active compound molecule and
§§ represents the bond to the antibody, and
L2-represents 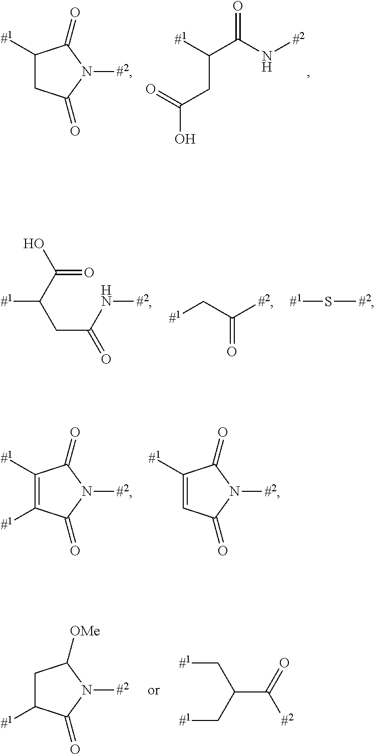
where
#¹ denotes the point of attachment to the sulphur atom of the antibody,
#² denotes the point of attachment to group L¹,
L¹ represents —(NR¹⁰)ₙ-(G1)ₒ-G2-,
where
R¹⁰ represents —H, —NH₂ or C₁-C₃-alkyl;
G1 represents —NH—C(═O)—;
n represents 0 or 1;
o represents 0 or 1; and
G2 represents a straight-chain or branched hydrocarbon chain having 1 to 100 (preferably 1 to 25) carbon atoms from aryl groups, and/or straight-chain and/or branched alkyl groups, and/or cyclic alkyl groups and which may be interrupted once or more than once, identically or differently by —O—, —S—, —S(═O)—, —S(═O)₂—, —NH—, —C(═O)—, —N—CH₃—, —NHNH—, —S(═O)₂—NHNH—, —NH—C(═O)—, —C(═O)—NH—, —C(═O)—NHNH— and a 5- to 10-membered aromatic or non-aromatic heterocycle having 1 to 4 identical or different heteroatoms and/or hetero groups, selected from the group consisting of N, O and S, —S(═O)— or —S(═O)₂—,
where straight chain or branched hydrocarbon chain may optionally be substituted by —NH—C(═O)—NH₂, —COOH, —OH, —NH₂, NH—CNNH₂, sulphonamide, sulphone, sulphoxide or sulphonic acid,
or represents one of the groups below: 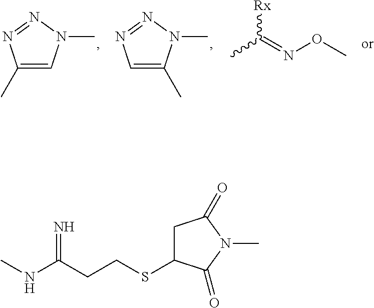
where R^{x} represents —H, C₁-C₃-alkyl or phenyl.

16: Conjugate according to claim 15 where L2 is represented by one or both of the formulae below: 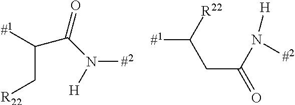
where
#¹ denotes the point of attachment to the sulphur atom of the binder,
#² denotes the point of attachment to group L¹,
R²² represents —COOH and
more than 80% (based on the total number of bonds of the linker to the binder) of the bonds to the sulphur atom of the binder are present in one of these two structures.

17: Conjugate according to claim 15 where L¹ has the formulae below: 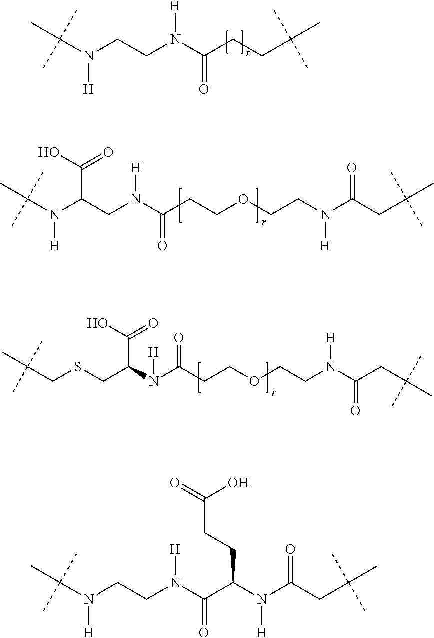
in which
r represents a number from 0 to 8.

18: Conjugate according to claim 1 where the linker -L- is attached to a cysteine side chain or a cysteine residue and has the formula below: 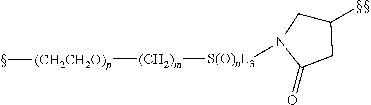
where
§ represents the bond to the active compound molecule and
§§ represents the bond to the antibody,
m represents 0, 1, 2 or 3;
n represents 0, 1 or 2;
p represents 0 to 20; and
L3 represents 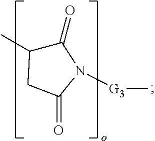
where
o represents 0 or 1; and
G3 represents a straight-chain or branched hydrocarbon chain having 1 to 100 (preferably 1 to 25) carbon atoms from aryl groups, and/or straight-chain and/or branched alkyl groups, and/or cyclic alkyl groups and which may be interrupted once or more than once, identically or differently by —O—, —S—, —S(═O)—, —S(═O)₂—, —NH—, —C(═O)—, —N—CH₃—, —NHNH—, —S(═O)₂—NHNH—, —NH—C(═O)—, —C(═O)—NH—, —C(═O)—NHNH— and a 5- to 10-membered aromatic or non-aromatic heterocycle having 1 to 4 identical or different heteroatoms and/or hetero groups, selected from the group consisting of N, O and S, —S(═O)— or —S(═O)₂—, where the straight-chain or branched hydrocarbon chain may optionally be substituted by —NH—C(═O)NH₂, —COOH, —OH, —NH₂, —NH—CNNH₂, sulphonamide, sulphone, sulphoxide or sulphonic acid.

19: Conjugate according to claim 1 preceding claims, where the conjugate has one of the formulae below: 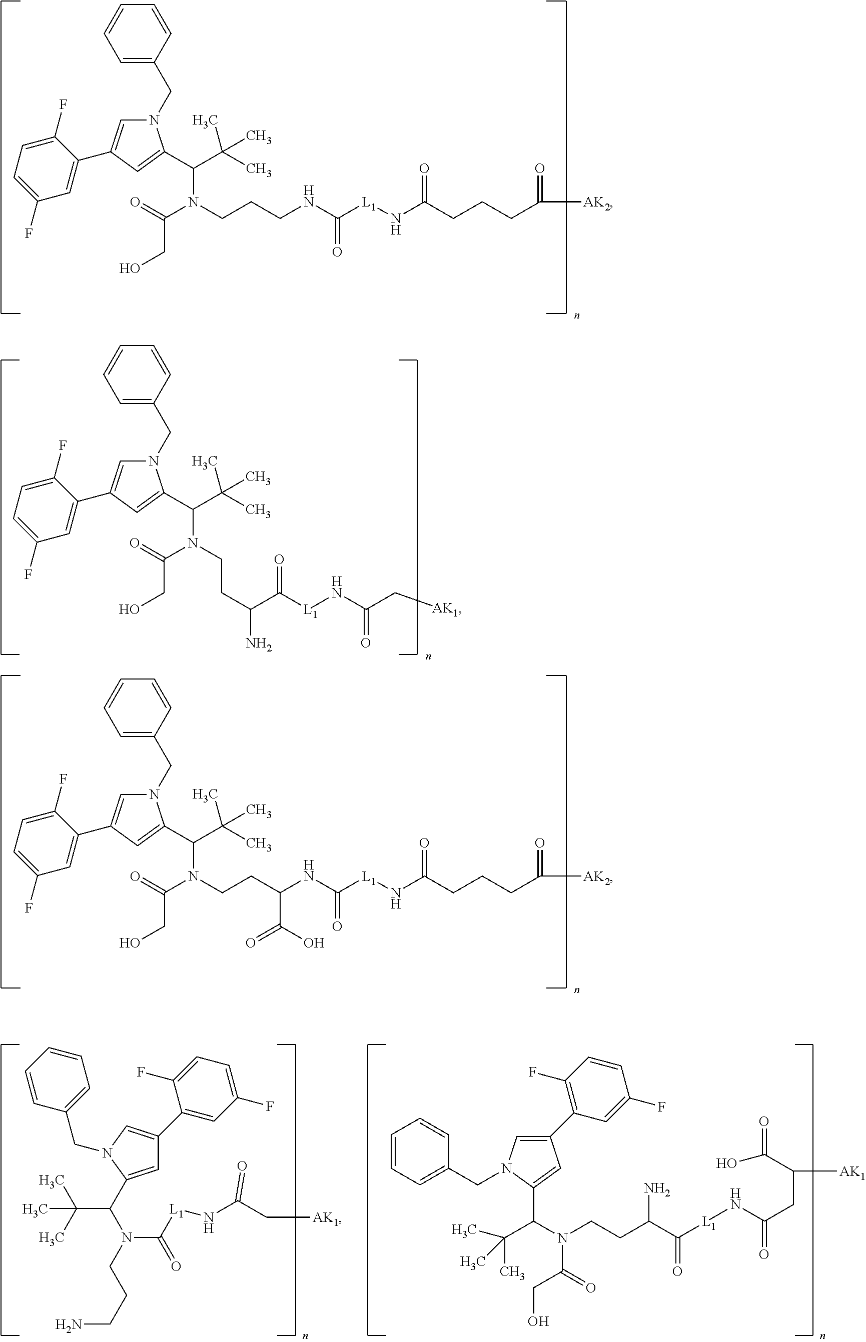 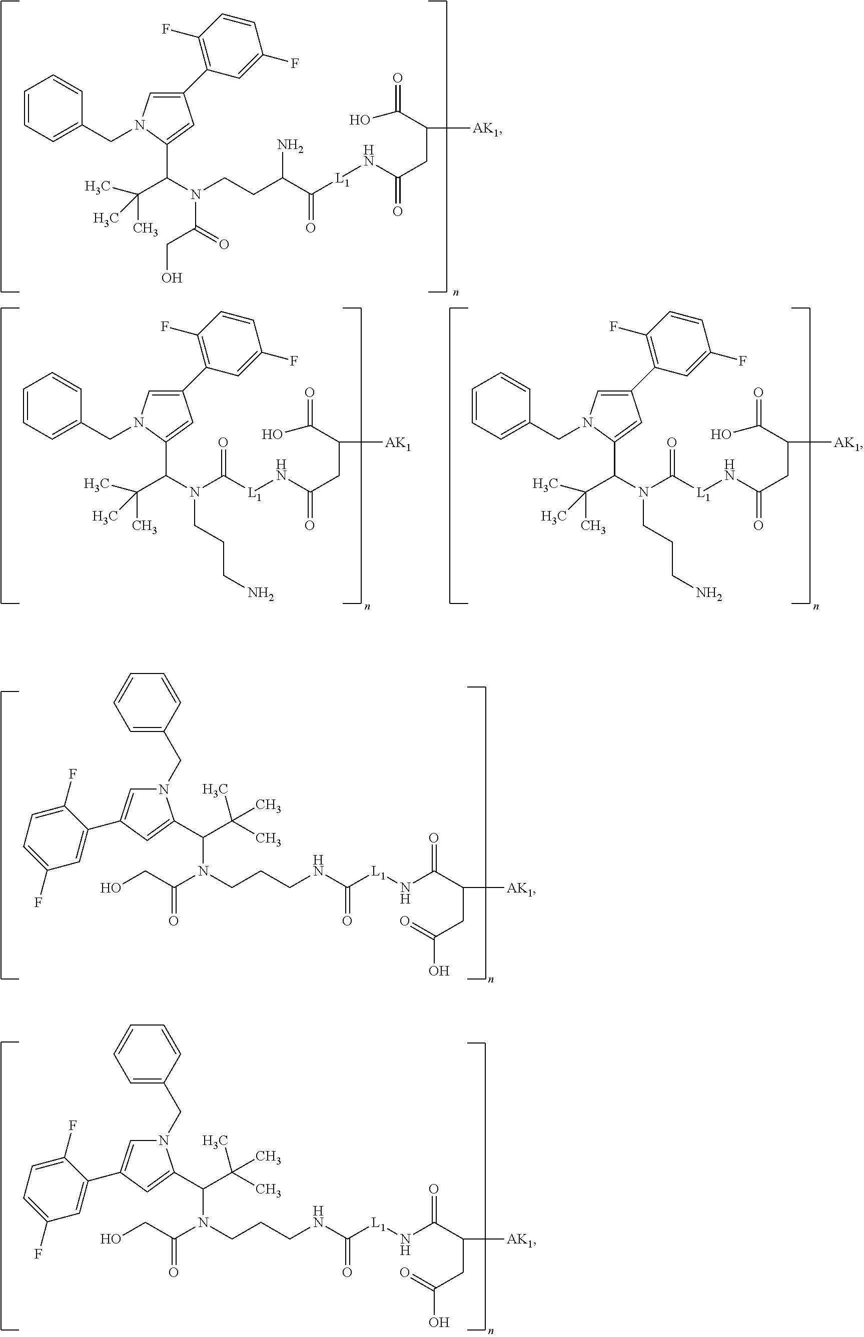 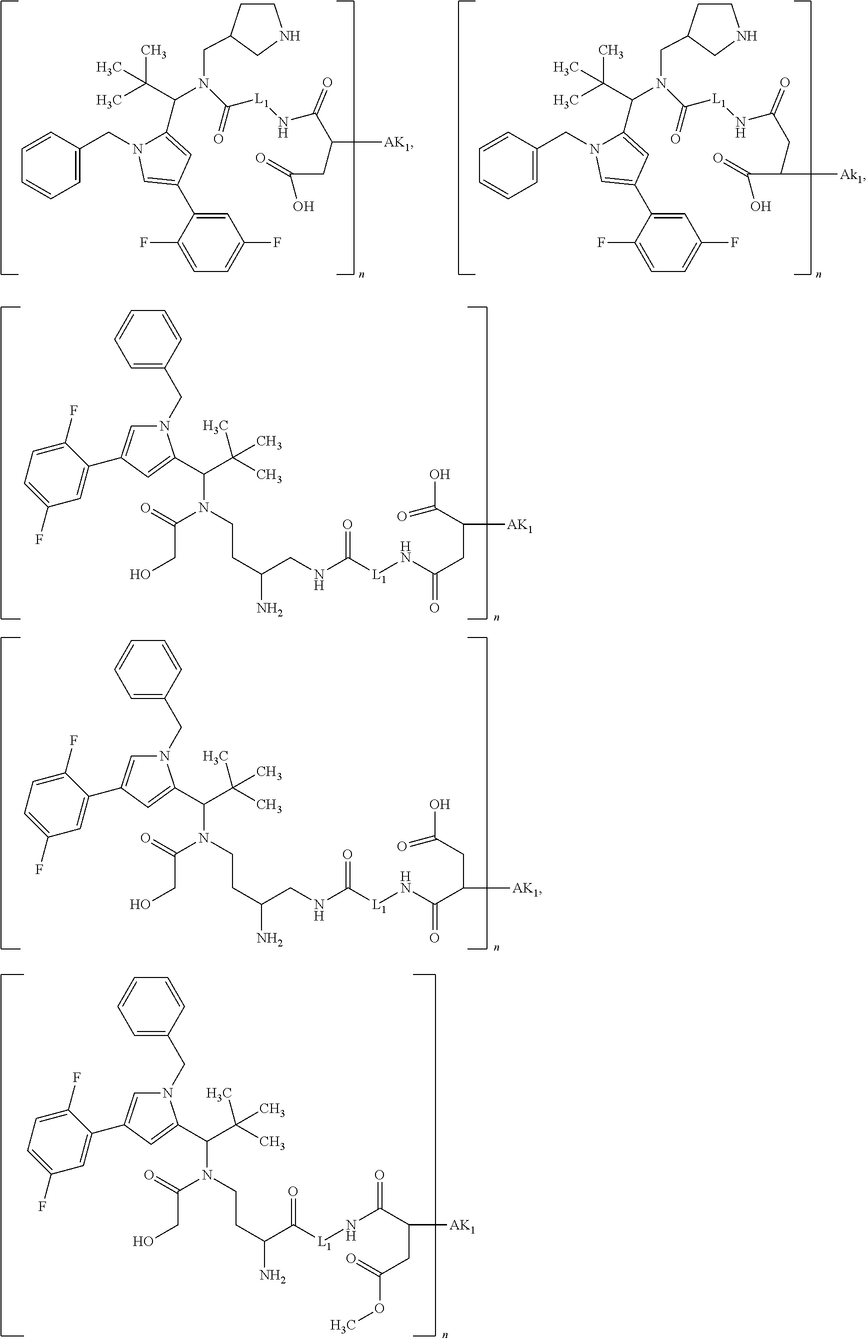 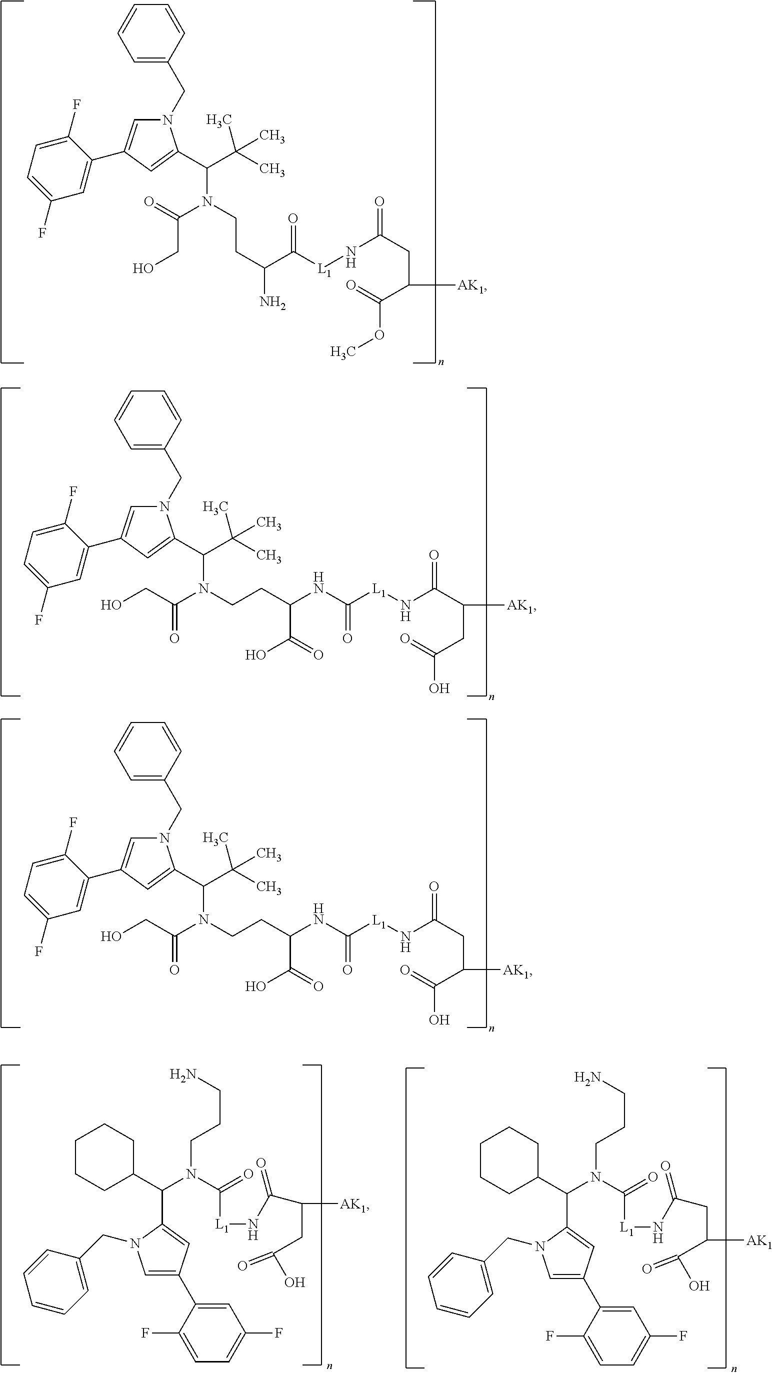 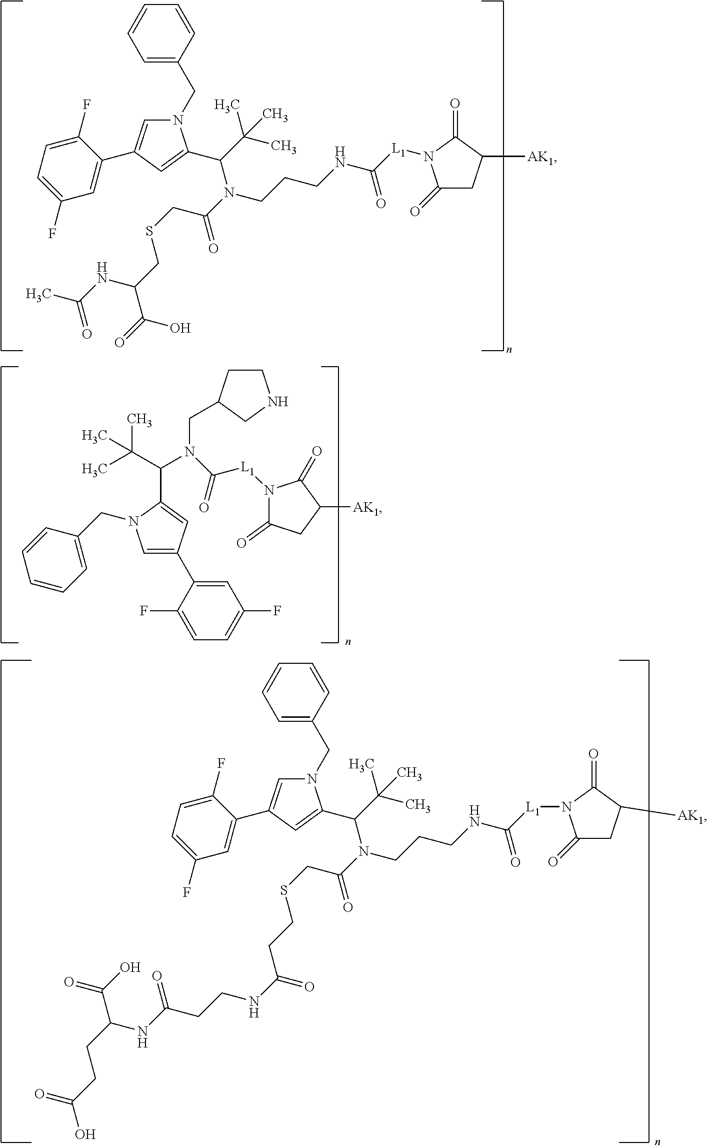 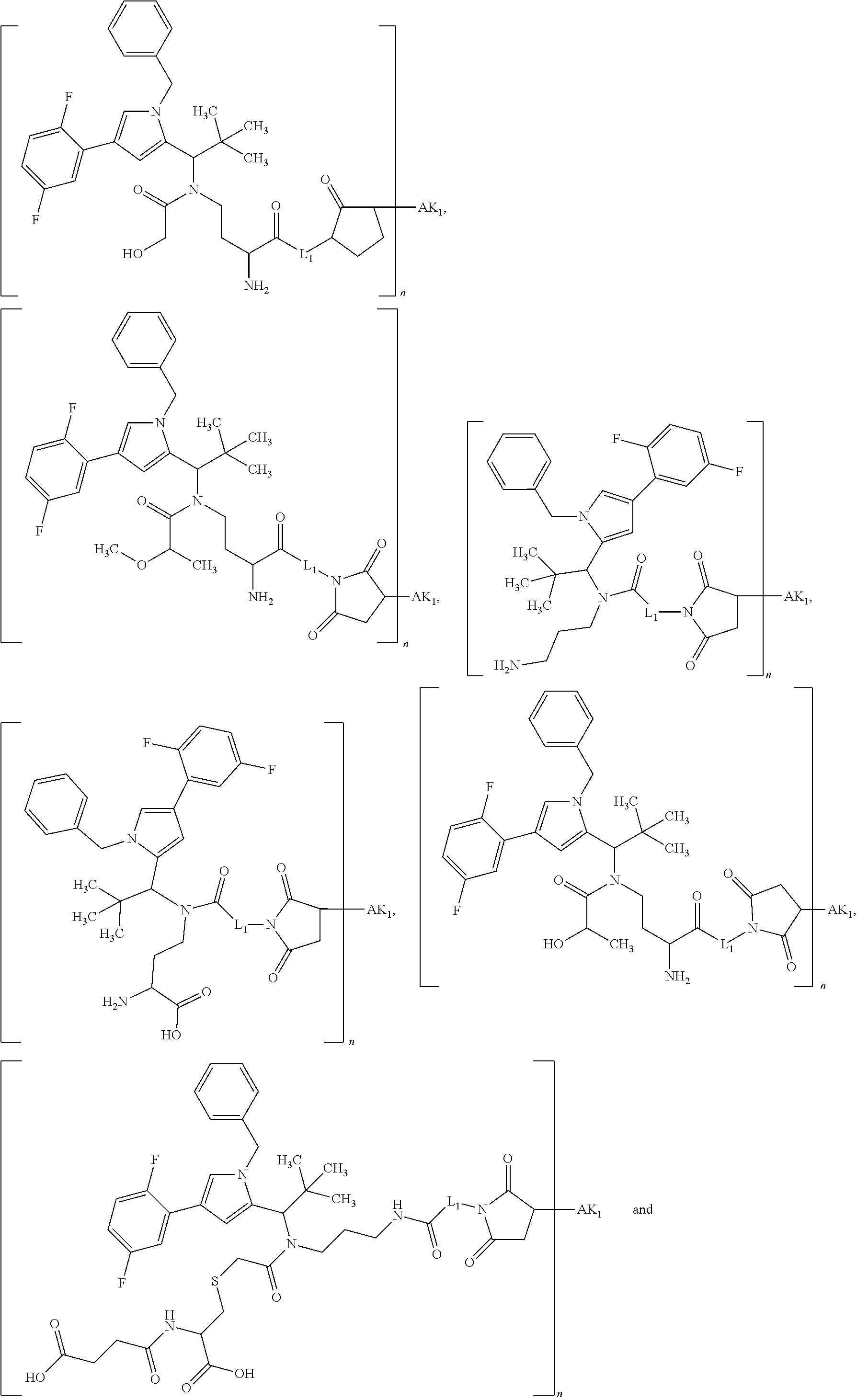 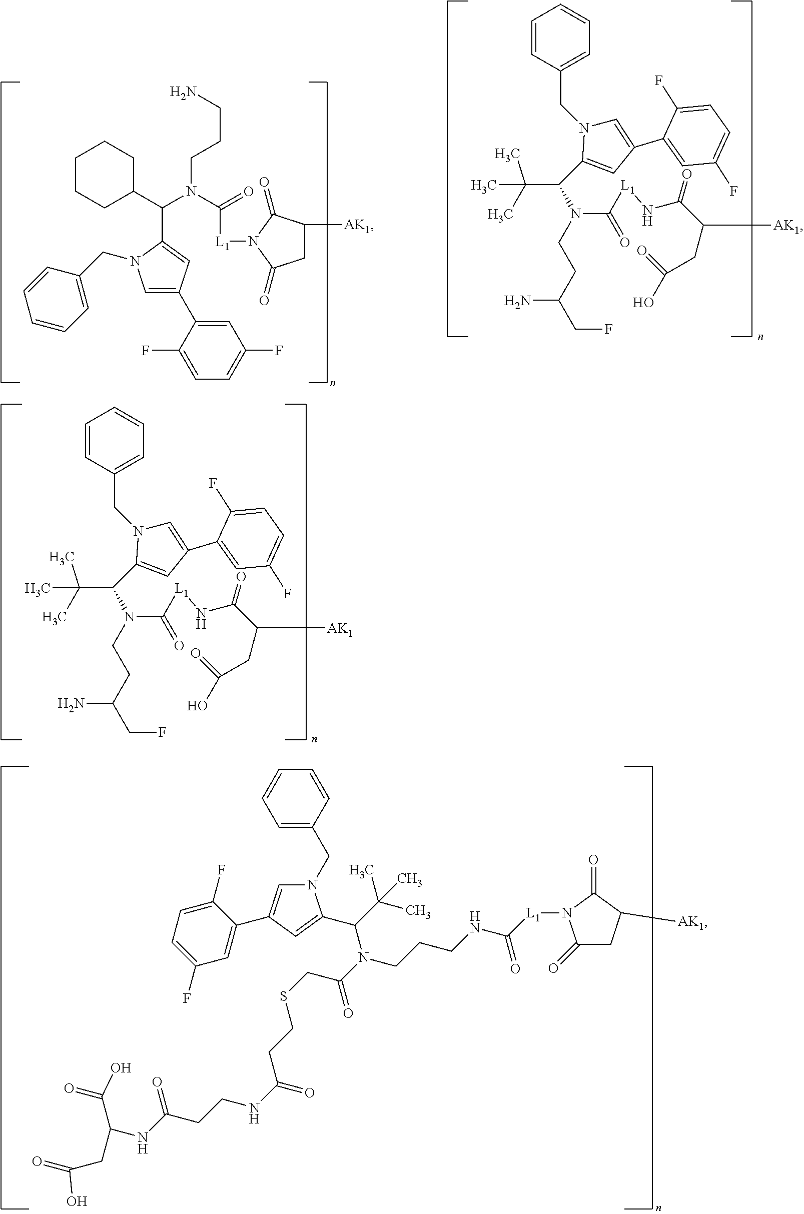 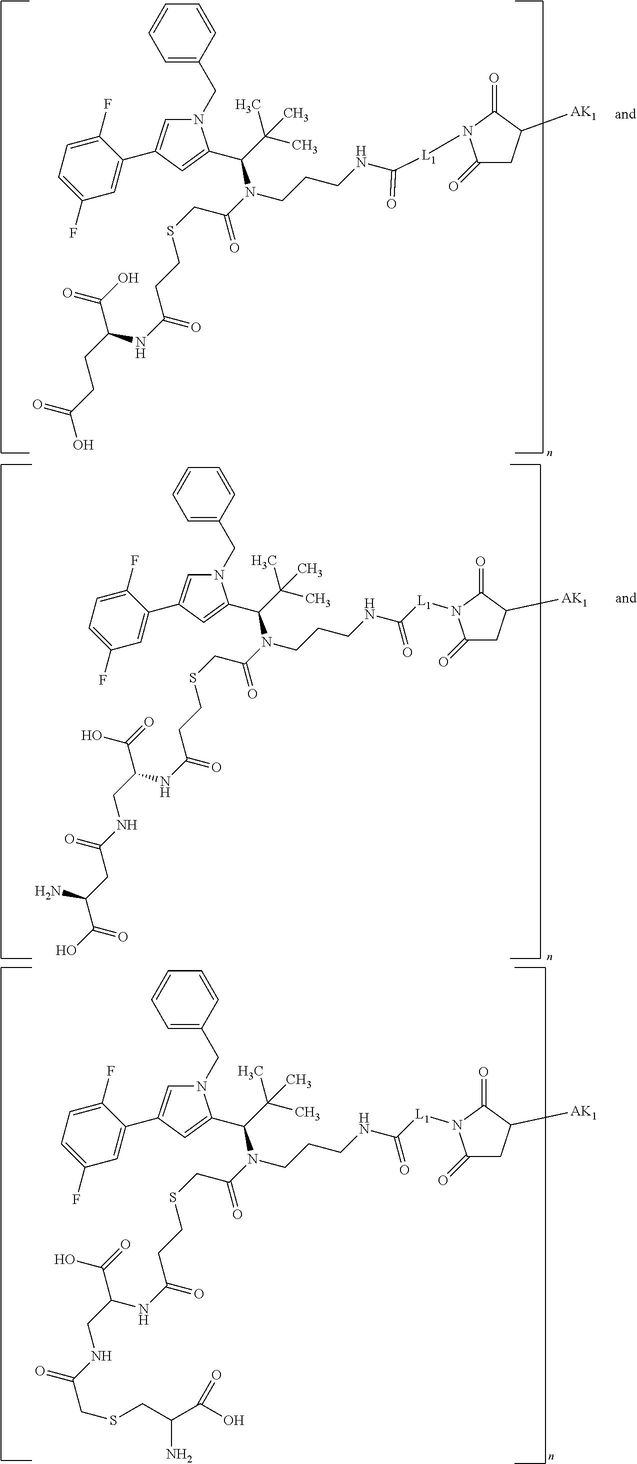
where
AK1 represents a moderately agonistically or non-agonistically acting anti-TWEAKR antibody which is attached via cystein and AK2 represents a moderately agonistically or non-agonistically acting anti-TWEAKR antibody which is attached via lysine,
n represents a number from 1 to 20; and
L₁ represents a straight-chain or branched hydrocarbon chain which has 1 to 30 carbon atoms and may be interrupted once or more than once, identicially or differently, by —O—, —S—, —C(═O)—, —S(═O)₂—, —NH—, cyclopentyl, piperidinyl, phenyl,
where the straight-chain or branched hydrocarbon chain may be substituted by —COOH or —NH₂,
and its salts, solvates, salts of the solvates and epimers.

20: Conjugate according to claim 19, where the linker L₁ represents the group
§—NH—(CH₂)₂-§§;
§—NH—(CH₂)₆-§§;
§—NH—(CH₂)₂—O—(CH₂)₂-§§;
§—NH—CH(COOH)—(CH₂)₄-§§
§—NH—NH—C(═O)—(CH₂)₅-§§;
§—NH—(CH₂)₂—C(═O)—O—(CH₂)₂-§§;
§—NH—(CH₂)₂—C(═O)—NH—(CH₂)₂-§§;
§—NH—(CH₂)₂—NH—C(═O)—CH₂-§§;
§—NH—(CH₂)₃—NH—C(═O)—CH₂-§§;
§—NH—(CH₂)₂—NH—C(═O)—(CH₂)₂-§§;
§—NH—(CH₂)₂—NH—C(═O)—(CH₂)₅-§§;
§—NH—(CH₂)₂—NH—C(═O)—CH(CH₃)—§§;
§—NH—(CH₂)₂—O—(CH₂)₂—NH—C(═O)—CH₂-§§;
§—NH—CH(COOH)—CH₂—NH—C(═O)—CH₂-§§;
§—NH—CH(COOH)—(CH₂)₂—NH—C(═O)—CH₂-§§;
§—NH—CH(COOH)—(CH₂)₄—NH—C(═O)—CH₂-§§;
§—NH—CH(COOH)—CH₂—NH—C(═O)—(CH₂)₂-§§;
§—NH—(CH₂)₂—NH—C(═O)—CH(C₂H₄COOH)—§§;
§—NH—(CH₂)₂—NH—C(═O)—((CH₂)₂—O)₃—(CH₂)₂-§§;
§—NH—(CH₂)₂—S(═O)₂—(CH₂)₂—NH—C(═O)—CH₂-§§;
§—NH—(CH₂)₂—NH—C(═O)—CH₂—NH—C(═O)—CH₂-§§;
§—NH—(CH₂)₃—NH—C(═O)—CH₂—NH—C(═O)—CH₂-§§;
§—NH—CH(COOH)—CH₂—NH—C(═O)—CH(CH₂COOH)—§§;
§—NH—(CH₂)₂—NH—C(═O)—CH(C₂H₄COOH)—NH—C(═O)—CH₂-§§;
§—NH—CH(COOH)—CH₂—NH—C(═O)—(CH₂)₂—NH—C(═O)—CH₂-§§;
§—NH—(CH₂)₂—NH—C(═O)—(CH₂)₂—CH(COOH)—NH—C(═O)—CH₂-§§;
§—NH—CH(COOH)—CH₂—NH—C(═O)—CH(CH₂OH)—NH—C(═O)—CH₂-§§;
§—NH—CH[C(═O)—NH—(CH₂)₂—O)₄—(CH₂)₂COOH]—CH₂—NH—C(═O)—CH₂-§§;
§—NH—CH(COOH)—CH₂—NH—C(═O)—((CH₂)₂—O)₄—(CH₂)₂—NH—C(═O)—CH₂-§§;
§—NH—(CH₂)₄—CH(COOH)—NH—C(═O)—CH(CH₃)—NH—C(═O)—CH(isoC₃H₇)—§§;
§—NH—(CH₂)₄—CH(COOH)—NH—C(═O)—CH(CH₃)—NH—C(═O)—CH(isoC₃H₇)—NH—C(═O)—(CH₂)₅-§§;
§—NH—(CH₂)₂—C(═O)—NH—(CH₂)₄—CH(COOH)—NH—C(═O)—CH(CH₃)—NH—C(═O)—CH(isoC₃H₇)—NH—C(═O)—CH₂-§§;
§—NH—(CH₂)₂—C(═O)—NH—(CH₂)₄—CH(COOH)—NH—C(═O)—CH(CH₃)—NH—C(═O)—CH(isoC₃H₇)—NH—C(═O)—(CH₂)₅-§§;
§—NH—(CH₂)₄—CH(COOH)—NH—C(═O)— CH[(CH₂)₃—NH—C(═O)—NH₂]—NH—C(═O)—CH(isoC₃H₇)—NH—C(═O)—(CH₂)₅-§§;
§—NH—(CH₂)₂—NH—C(═O)—(CH₂)₂—CH(COOH)—NH—C(═O)— CH(CH₃)—NH—C(═O)—CH(isoC₃H₇)—NH—C(═O)—(CH₂)₅-§§;
§—NH—CH(CH₃)—C(═O)—NH—(CH₂)₄—CH(COOH)—NH—C(═O)—CH(CH₃)—NH—C(═O)—CH(isoC₃H₇)—NH—C(═O)—(CH₂)₅-§§;
§—NH—(CH₂)₂—C(═O)—NH—(CH₂)₄—CH(COOH)—NH—C(═O)—CH[(CH₂)₃—NH—C(═O)—NH₂]—NH—C(═O)—CH(isoC₃H₇)—NH—C(═O)—(CH₂)₅-§§;
§—NH 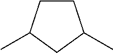
C(═O)—NH—(CH₂)₂-§§;
§—NH 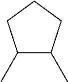
C(═O)—NH—(CH₂)₂—NH—C(═O)—CH₂-§§;
§—NH 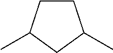
C(═O)—NH—(CH₂)₄—CH(COOH)—NH—C(═O)—CH[(CH₂)₃—NH—C(═O)—NH₂]—NH—C(═O)—CH(isoC₃H₇)—NH—C(═O)—(CH₂)₅-§§;
§—NH 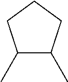
C(═O)—NH—(CH₂)₄—CH(COOH)—NH—C(═O)—CH[(CH₂)₃—NH—C(═O)—NH₂]—NH—C(═O)—CH(isoC₃Hᵥ)—NH—C(═O)—(CH₂)₅-§§;
§—NH 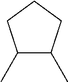
C(═O)—NH—(CH₂)₄—CH(COOH)—NH—C(═O)—CH(CH₃)—NH—C(═O)—CH(isoC₃H₇)—NH—C(═O)—(CH₂)₅-§§;
§—NH—(CH₂)₂—C(═O)—NH—CH(isoC₃H₇)—C(═O)—NH—CH[(CH₂)₃—NH—C(═O)—NH₂]—C(═O)—O 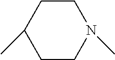
(═O)—CH₂-§§;
§—NH—(CH₂)₂—C(═O)—NH—CH(isoC₃H₇)—C(═O)—NH—CH(CH₃)—C(═O)—O 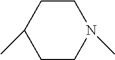
C(═O)—CH₂-§§;
§—NH—(CH₂)₂—NH—C(═O) 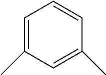
§§;
§—NH—CH(COOH)—CH₂—NH—C(═O 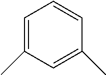
§§;
§—NH—(CH₂)₂—C(═O)—NH—CH(CH₃)—C(═O)—NH—CH[(CH₂)₃—NH—C(═O)—NH₂]—C(═O)—NH 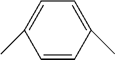
§§;
§—(CH₂)₂—C(═O)—NH—(CH₂)₂-§§;
§—(CH₂)₂—C(═O)—NH—(CH₂)₂—NH—C(═O)—CH₂-§§;
§—CH(CH₃)—NH—C(═O)—CH(isoC₃H₇)—§§;
§—CH(CH₃)—NH—C(═O)—CH(isoC₃H₇)—NH—C(═O)—CH₂-§§;
§—CH(CH₃)—NH—C(═O)—CH(isoC₃H₇)—NH—C(═O)—(CH₂)₅-§§;
§—(CH₂)₂—C(═O)—NH—((CH₂)₂—O)₄—(CH₂)₂—NH—C(═O)—CH₂-§§;
§—CH(CH₃)—NH—C(═O)—CH(isoC₃H₇)—NH—C(═O)—((CH₂)₂—O)₄—(CH₂)₂—NH—C(═O)—(CH₂)₂-§§;
§ 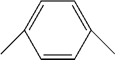
NH—C(═O)—CH(CH₃)—NH—C(═O)—CH(isoC₃H₇)—NH—C(═O)—((CH₂)₂—O)₄—(CH₂)₂—NH—C(═O)—(CH₂)₂-§§;
§—CH₂—S—(CH₂)₂—C(═O)—NH—(CH₂)₂-§§;
§—CH₂—S—(CH₂)₅—C(═O)—NH—(CH₂)₂-§§;
§—CH₂—S—CH₂CH(COOH)—NH—C(═O)—CH₂-§§;
§—CH₂—S—CH₂CH(COOH)—NH—C(═O)—(CH₂)₅-§§;
§—CH₂—S—(CH₂)₂—C(═O)—NH—((CH₂)₂—O)₂—(CH₂)₂-§§;
§—CH₂—S—(CH₂)₂—C(═O)—NH—((CH₂)₂—O)₂—(CH₂)₅-§§;
§—CH₂—S—(CH₂)₂—C(═O)—NH—(CH₂)₂—NH—C(═O)—CH₂-§§;
§—CH₂—S—(CH₂)₂—C(═O)—NH—(CH₂)₂—NH—C(═O)—CH₅-§§;
§—CH₂—S—(CH₂)₂—C(═O)—NH—CH(COOH)—CH₂—NH—C(═O)—CH₂-§§;
§—CH₂—S—CH₂CH(NH₂)—C(═O)—NH—(CH₂)₂—NH—C(═O)—(CH₂)₅-§§;
§—CH₂—S—(CH₂)₂—C(═O)—NH—((CH₂)₂—O)₂—(CH₂)₂—NH—C(═O)—CH₂-§§;
§—CH₂—S—(CH₂)₂—C(═O)—NH—((CH₂)₂—O)₄—(CH₂)₂—NH—C(═O)—CH₂-§§;
§—CH₂—S—(CH₂)₂—C(═O)—NH—((CH₂)₂—O)₂—(CH₂)₂—NH—C(═O)—(CH₂)₅-§§;
§—CH₂—S—(CH₂)₂—C(═O)—NH—((CH₂)₂—O)₄—(CH₂)₂—NH—C(═O)—(CH₂)₅-§§;
§—CH₂—S—CH₂CH(COOH)—NH—C(═O)—((CH₂)₂—O)₂—(CH₂)₂—NH—C(═O)—CH₂-§§;
§—CH₂—S—CH₂CH(COOH)—NH—C(═O)—((CH₂)₂—O)₄—(CH₂)₂—NH—C(═O)—CH₂-§§;
§—CH₂—S—CH₂CH(COOH)—NH—C(═O)—((CH₂)₂—O)₄—(CH₂)₂—NH—C(═O)—(CH₂)₂-§§;
§—CH₂—S—(CH₂)₂—CH(COOH)—NH—C(═O)—((CH₂)₂—O)₄—(CH₂)₂—NH—C(═O)—(CH₂)₂-§§;
§—CH₂—S—(CH₂)₂—C(═O)—NH—CH(C₂H₄COOH)—C(═O)—NH—(CH₂)₂—NH—C(═O)—CH₂-§§;
§—CH₂—S—CH₂CH[NH—C(═O)—(CH₂)₂—COOH]—C(═O)—NH—(CH₂)₂—NH—C(═O)—CH₂-§§;
§—CH₂—S—CH₂CH[NH—C(═O)—((CH₂)₂—O)₄—CH₃]—C(═O)—NH—(CH₂)₂—NH—C(═O)—CH₂-§§;
§—CH₂—S—CH₂CH(COOH)—NH—C(═O)—CH(CH₃)—NH—C(═O)—CH(isoC₃H₇)—NH—C(═O)—CH₂-§§;
§—CH₂—S—CH₂CH[NH—C(═O)—(CH₂)₂—COOH]—C(═O)—NH—(CH₂)₂—S(═O)₂—(CH2)2-NH—C(═O)—CH₂-§§;
§—CH₂—S—CH₂CH[NH—C(═O)—(CH₂)₂—COOH]—C(═O)—NH—((CH₂)₂—O)₄—(CH₂)₂—NH—C(═O)—CH₂-§§;
§—CH₂—S—CH₂CH[C(═O)—NH—(CH₂)₂—COOH]—NH—C(═O)—((CH₂)₂—O)₄—(CH₂)₂—NH—C(═O)—CH₂-§§;
§—CH₂—S—CH₂CH(COOH)—NH—C(═O)—(CH₂)₂CH(COOH)—NH—C(═O)—((CH₂)₂—O)₄—(CH₂)₂—NH—C(═O)—CH₂-§§
or
§—CH₂—S—CH₂CH(COOH)—NH—C(═O)—CH[(CH₂)₂—COOH]—NH—C(═O)—((CH₂)₂—O)₄—(CH₂)₂—NH—C(═O)—(CH₂)₂-§§,
where
§ represents the bond to the active component molecule and
§§ represents the bond to the antibody and
isoC₃H₇ represents an isopropyl radical,
and its salts, solvates, salts of the solvates and epimers.

21: Conjugate according to claim 1, where the conjugate has one of the formulae below: 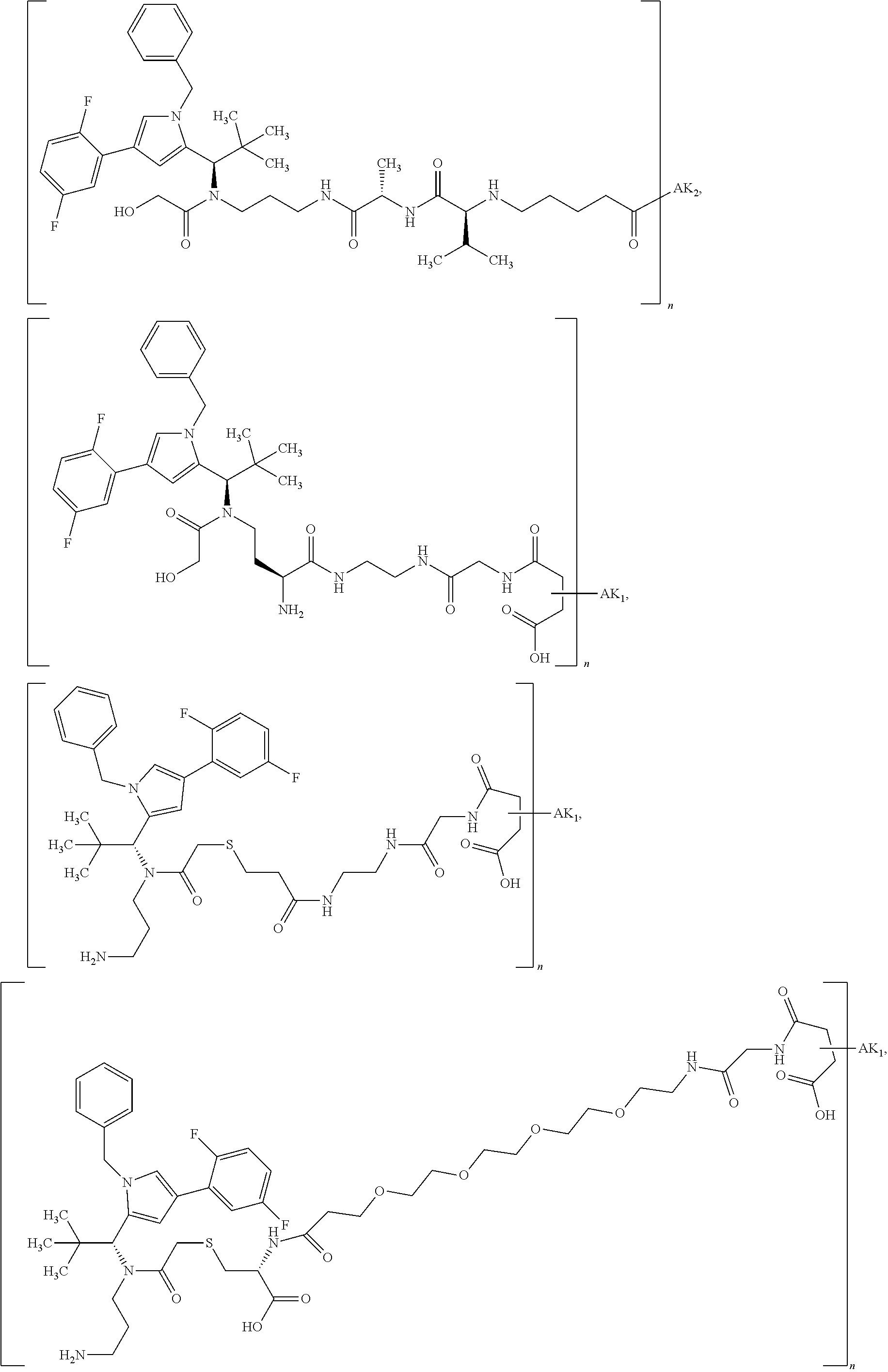 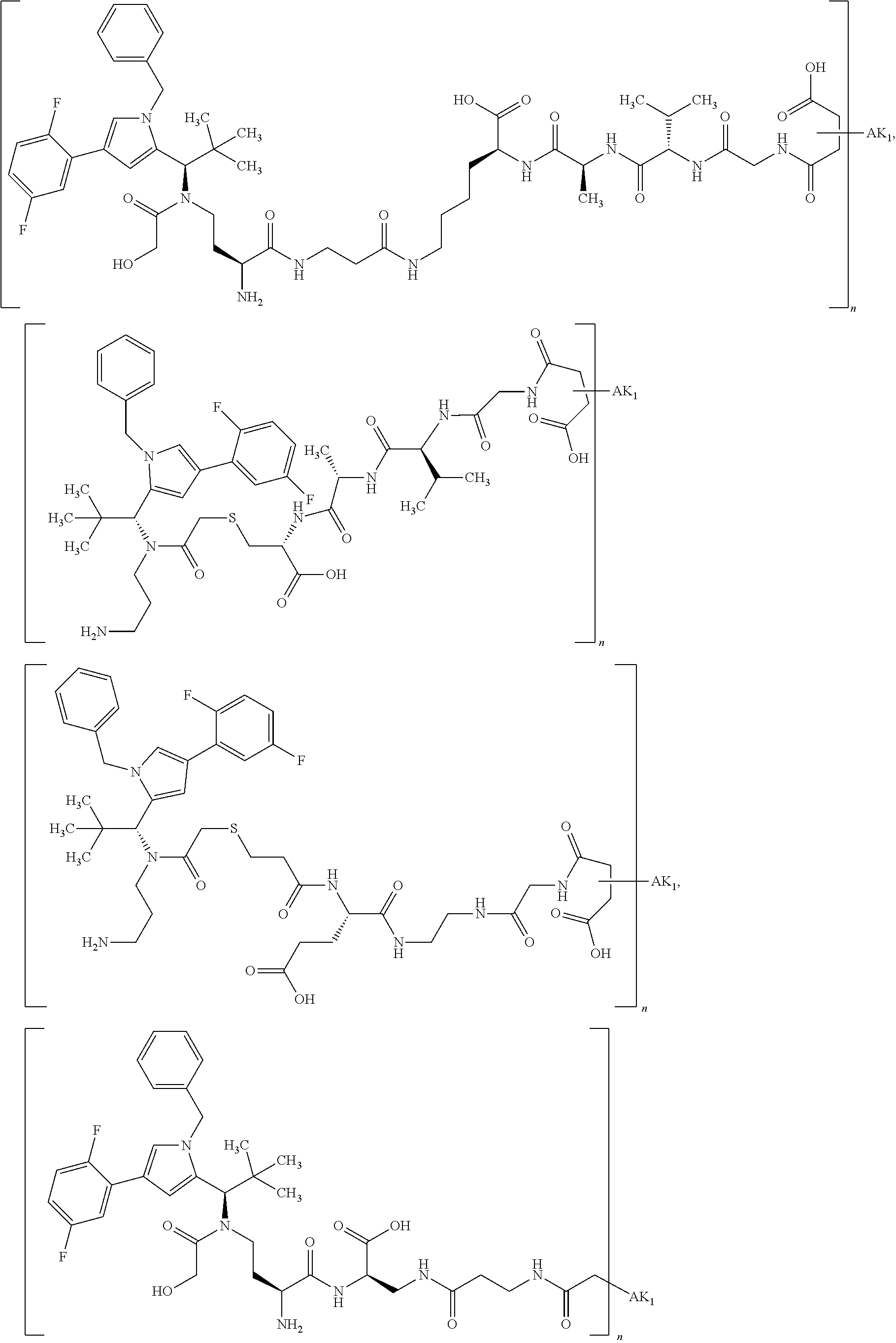 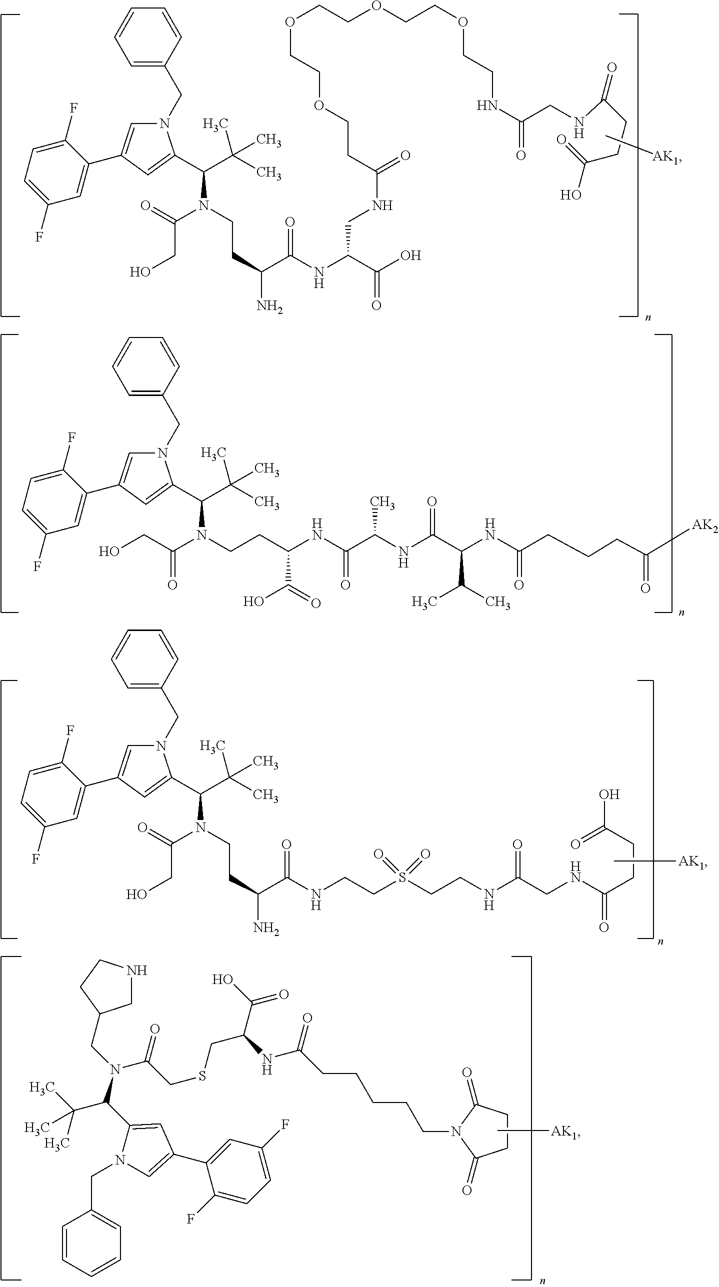 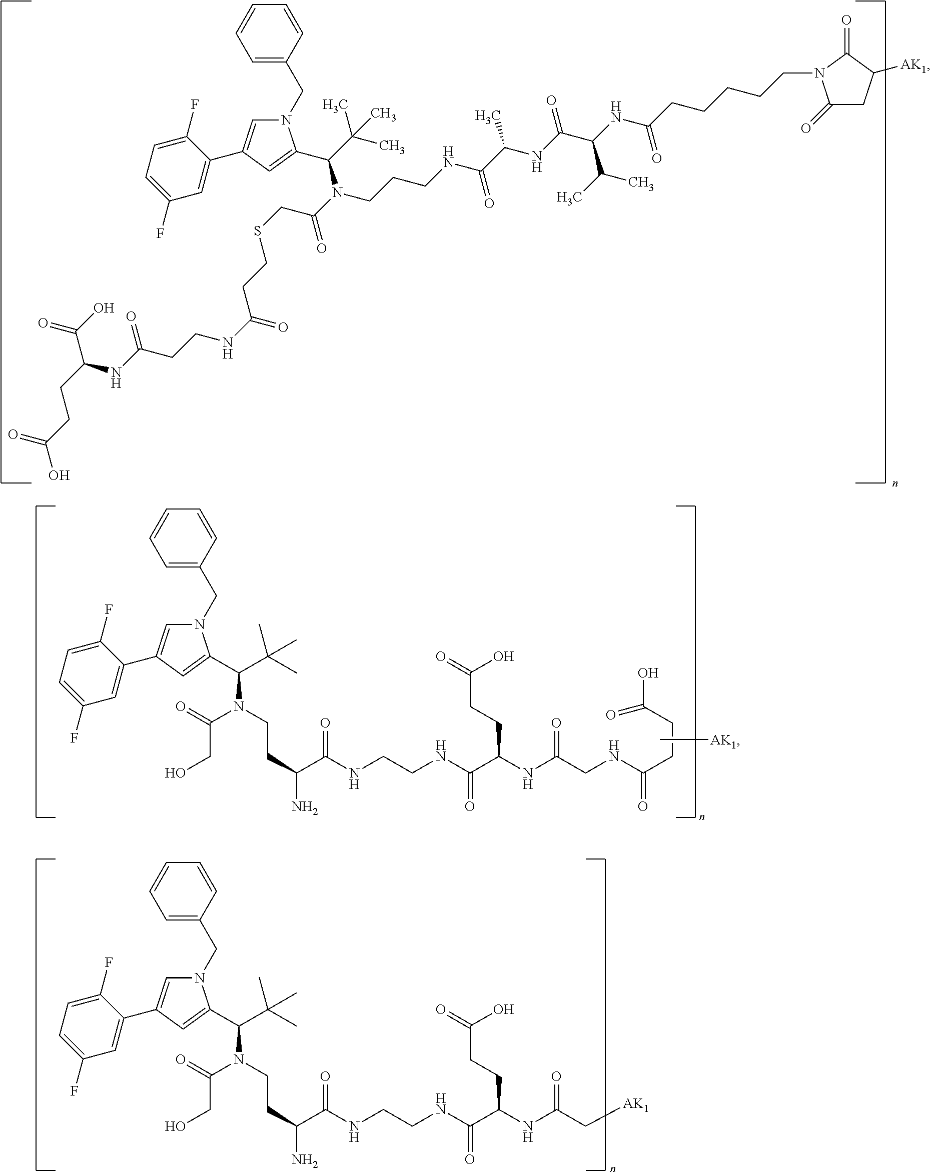
where
AK1 represents a moderately agonistically or non-agonistically acting anti-TWEAKR antibody attached via cystein and AK2 represents a moderately agonistically or non-agonistically acting anti-TWEAKR antibody attached via lysine and
n represents a number from 1 to 20.

22: Conjugate according to claim 1 where the moderately agonistically or non-agonistically acting anti-TWEAKR antibody is ITEM-4 or a chimeric or humanized variant of the ITEM-4 antibody or an antigen-binding fragment thereof.

23: Conjugate according to claim 1 where the moderately agonistically or non-agonistically acting anti-TWEAKR antibody or the antigen-binding fragment thereof comprises:
a variable heavy chain comprising the variable CDR1 sequence of the heavy chain, as shown in SEQ ID NO: 2, the variable CDR2 sequence of the heavy chain, as shown in SEQ ID NO: 3, and the variable CDR3 sequence of the heavy chain, as shown in SEQ ID NO: 4, and
a variable light chain comprising the variable CDR1 sequence of the light chain, as shown in SEQ ID NO: 6, the variable CDR2 sequence of the light chain, as shown in SEQ ID NO: 7, and the variable CDR3 sequence of the light chain, as shown in SEQ ID NO: 8, or
a variable heavy chain comprising the variable CDR1 sequence of the heavy chain, as shown in SEQ ID NO: 12, the variable CDR2 sequence of the heavy chain, as shown in SEQ ID NO: 13, and the variable CDR3 sequence of the heavy chain, as shown in SEQ ID NO: 14, and
a variable light chain comprising the variable CDR1 sequence of the light chain, as shown in SEQ ID NO: 16, the variable CDR2 sequence of the light chain, as shown in SEQ ID NO: 17, and the variable CDR3 sequence of the light chain, as shown in SEQ ID NO: 18, or
a variable heavy chain comprising the variable CDR1 sequence of the heavy chain, as shown in SEQ ID NO: 22, the variable CDR2 sequence of the heavy chain, as shown in SEQ ID NO: 23, and the variable CDR3 sequence of the heavy chain, as shown in SEQ ID NO: 24, and
a variable light chain comprising the variable CDR1 sequence of the light chain, as shown in SEQ ID NO: 26, the variable CDR2 sequence of the light chain, as shown in SEQ ID NO: 27, and the variable CDR3 sequence of the light chain, as shown in SEQ ID NO: 28, or
a variable heavy chain comprising the variable CDR1 sequence of the heavy chain, as shown in SEQ ID NO: 32, the variable CDR2 sequence of the heavy chain, as shown in SEQ ID NO: 33, and the variable CDR3 sequence of the heavy chain, as shown in SEQ ID NO: 34, and
a variable light chain comprising the variable CDR1 sequence of the light chain, as shown in SEQ ID NO: 36, the variable CDR2 sequence of the light chain, as shown in SEQ ID NO: 37, and the variable CDR3 sequence of the light chain, as shown in SEQ ID NO: 38, or
a variable heavy chain comprising the variable CDR1 sequence of the heavy chain, as shown in SEQ ID NO: 52, the variable CDR2 sequence of the heavy chain, as shown in SEQ ID NO: 53, and the variable CDR3 sequence of the heavy chain, as shown in SEQ ID NO: 54, and
a variable light chain comprising the variable CDR1 sequence of the light chain, as shown in SEQ ID NO: 56, the variable CDR2 sequence of the light chain, as shown in SEQ ID NO: 57, and the variable CDR3 sequence of the light chain, as shown in SEQ ID NO: 58, or
a variable heavy chain comprising the variable CDR1 sequence of the heavy chain, as shown in SEQ ID NO: 62, the variable CDR2 sequence of the heavy chain, as shown in SEQ ID NO: 63, and the variable CDR3 sequence of the heavy chain, as shown in SEQ ID NO: 64, and
a variable light chain comprising the variable CDR1 sequence of the light chain, as shown in SEQ ID NO: 66, the variable CDR2 sequence of the light chain, as shown in SEQ ID NO: 67, and the variable CDR3 sequence of the light chain, as shown in SEQ ID NO: 68, or
a variable heavy chain comprising the variable CDR1 sequence of the heavy chain, as shown in SEQ ID NO: 72, the variable CDR2 sequence of the heavy chain, as shown in SEQ ID NO: 73, and the variable CDR3 sequence of the heavy chain, as shown in SEQ ID NO: 74, and
a variable light chain comprising the variable CDR1 sequence of the light chain, as shown in SEQ ID NO: 76, the variable CDR2 sequence of the light chain, as shown in SEQ ID NO: 77, and the variable CDR3 sequence of the light chain, as shown in SEQ ID NO: 78, or
a variable heavy chain comprising the variable CDR1 sequence of the heavy chain, as shown in SEQ ID NO: 82, the variable CDR2 sequence of the heavy chain, as shown in SEQ ID NO: 83, and the variable CDR3 sequence of the heavy chain, as shown in SEQ ID NO: 84, and
a variable light chain comprising the variable CDR1 sequence of the light chain, as shown in SEQ ID NO: 86, the variable CDR2 sequence of the light chain, as shown in SEQ ID NO: 87, and the variable CDR3 sequence of the light chain, as shown in SEQ ID NO: 88.

24: Conjugate according to claim 1 where the moderately agonistically or non-agonistically acting anti-TWEAKR antibody or the antigen-binding fragment thereof comprises:
a variable sequence of the heavy chain, as shown in SEQ ID NO: 1, and also
a variable sequence of the light chain, as shown in SEQ ID NO: 5, or
a variable sequence of the heavy chain, as shown in SEQ ID NO: 11, and also
a variable sequence of the light chain, as shown in SEQ ID NO: 15, or
a variable sequence of the heavy chain, as shown in SEQ ID NO: 21, and also
a variable sequence of the light chain, as shown in SEQ ID NO: 25, or
a variable sequence of the heavy chain, as shown in SEQ ID NO: 31, and also
a variable sequence of the light chain, as shown in SEQ ID NO: 35, or
a variable sequence of the heavy chain, as shown in SEQ ID NO: 51, and also
a variable sequence of the light chain, as shown in SEQ ID NO: 55, or
a variable sequence of the heavy chain, as shown in SEQ ID NO: 61, and also
a variable sequence of the light chain, as shown in SEQ ID NO: 65, or
a variable sequence of the heavy chain, as shown in SEQ ID NO: 71, and also
a variable sequence of the light chain, as shown in SEQ ID NO: 75, or
a variable sequence of the heavy chain, as shown in SEQ ID NO: 81, and also
a variable sequence of the light chain, as shown in SEQ ID NO: 85.

25: Conjugate according to claim 1 where the moderately agonistically or non-agonistically acting anti-TWEAKR antibody is an IgG antibody.

26: Conjugate according to claim 1 where the moderately agonistically or non-agonistically acting anti-TWEAKR antibody comprises:
a sequence of the heavy chain, as shown in SEQ ID NO: 9, and also a sequence of the light chain, as shown in SEQ ID NO: 10, or
a sequence of the heavy chain, as shown in SEQ ID NO: 19, and also a sequence of the light chain, as shown in SEQ ID NO: 20, or
a sequence of the heavy chain, as shown in SEQ ID NO: 29, and also a sequence of the light chain, as shown in SEQ ID NO: 30, or
a sequence of the heavy chain, as shown in SEQ ID NO: 39, and also a sequence of the light chain, as shown in SEQ ID NO: 40, or
a sequence of the heavy chain, as shown in SEQ ID NO: 59, and also a sequence of the light chain, as shown in SEQ ID NO: 60, or
a sequence of the heavy chain, as shown in SEQ ID NO: 69, and also a sequence of the light chain, as shown in SEQ ID NO: 70, or
a sequence of the heavy chain, as shown in SEQ ID NO: 79, and also a sequence of the light chain, as shown in SEQ ID NO: 80, or
a sequence of the heavy chain, as shown in SEQ ID NO: 89, and also a sequence of the light chain, as shown in SEQ ID NO: 90.

27: Pharmaceutical composition comprising a conjugate according to claim 1 in combination with an inert non-toxic pharmaceutically suitable auxiliary.

28: A method for the treatment or prophylaxis of diseases, the method comprising administering to a patient in need thereof a conjugate according to claim 1.

29: A method for the treatment or prophylaxis of hyperproliferative or angiogenic disorders, the method comprising administering to a patient in need thereof a conjugate according to claim 1.


SEQUENCE LISTING (102 sequences)

SEQ ID NO: 1 - protein, 120 aa, Artificial Sequence
  TPP-7005 -  VH
QVQLVQSGAEVKKPGASVKISCKVSGYTFTDFIIAWVKQAPGKGLEWIGEIYPGTGRTYYSEKFRGKATLTADKSTSTAYMELSSLRSEDTAVYYCARRTIYYDYDGDYWGQGTTVTVSS

SEQ ID NO: 2 - protein, 5 aa, Artificial Sequence
  TPP-7005 -  HCDR1
DFIIA

SEQ ID NO: 3 - protein, 17 aa, Artificial Sequence
  TPP-7005 -  HCDR2
EIYPGTGRTYYSEKFRG

SEQ ID NO: 4 - protein, 11 aa, Artificial Sequence
  TPP-7005 -  HCDR3
RTIYYDYDGDY

SEQ ID NO: 5 - protein, 112 aa, Artificial Sequence
  TPP-7005 -  VL
DIVMTQSPLSLPVTPGEPASISCRSSKSLLHSNGITYLYWYLQKPGQSPQLLIYQMSNLASGVPDRFSSSGSGTDFTLKISRVEAEDVGVYYCAHNLELPWTFGGGTKVEIK

SEQ ID NO: 6 - protein, 16 aa, Artificial Sequence
  TPP-7005 -  LCDR1
RSSKSLLHSNGITYLY

SEQ ID NO: 7 - protein, 7 aa, Artificial Sequence
  TPP-7005 -  LCDR2
QMSNLAS

SEQ ID NO: 8 - protein, 9 aa, Artificial Sequence
  TPP-7005 -  LCDR3
AHNLELPWT

SEQ ID NO: 9 - protein, 449 aa, Artificial Sequence
  TPP-7005 -  Heavy Chain
QVQLVQSGAEVKKPGASVKISCKVSGYTFTDFIIAWVKQAPGKGLEWIGEIYPGTGRTYYSEKFRGKATLTADKSTSTAYMELSSLRSEDTAVYYCARRTIYYDYDGDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 219 aa, Artificial Sequence
  TPP-7005 -  Light Chain
DIVMTQSPLSLPVTPGEPASISCRSSKSLLHSNGITYLYWYLQKPGQSPQLLIYQMSNLASGVPDRFSSSGSGTDFTLKISRVEAEDVGVYYCAHNLELPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 120 aa, Artificial Sequence
  TPP-7006 -  VH
QVQLQQSGPELVKPGASVKMSCKASGYTFTDFIIAWVKQRTGQGLEWIGEIYPGTGRTYYSEKFRGKATLTADKSSNTAYMQLSSLTSVDSAVYFCARRTIYYDYDGDYWGQGTTLTVSS

SEQ ID NO: 12 - protein, 5 aa, Artificial Sequence
  TPP-7006 -  HCDR1
DFIIA

SEQ ID NO: 13 - protein, 17 aa, Artificial Sequence
  TPP-7006 -  HCDR2
EIYPGTGRTYYSEKFRG

SEQ ID NO: 14 - protein, 11 aa, Artificial Sequence
  TPP-7006 -  HCDR3
RTIYYDYDGDY

SEQ ID NO: 15 - protein, 112 aa, Artificial Sequence
  TPP-7006 -  VL
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAHNLELPWTFGGGTKLELK

SEQ ID NO: 16 - protein, 16 aa, Artificial Sequence
  TPP-7006 -  LCDR1
RSSKSLLHSNGITYLY

SEQ ID NO: 17 - protein, 7 aa, Artificial Sequence
  TPP-7006 -  LCDR2
QMSNLAS

SEQ ID NO: 18 - protein, 9 aa, Artificial Sequence
  TPP-7006 -  LCDR3
AHNLELPWT

SEQ ID NO: 19 - protein, 449 aa, Artificial Sequence
  TPP-7006 -  Heavy Chain
QVQLQQSGPELVKPGASVKMSCKASGYTFTDFIIAWVKQRTGQGLEWIGEIYPGTGRTYYSEKFRGKATLTADKSSNTAYMQLSSLTSVDSAVYFCARRTIYYDYDGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 219 aa, Artificial Sequence
  TPP-7006 -  Light Chain
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAHNLELPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 120 aa, Artificial Sequence
  TPP-7007 -  VH
QVQLVQSGAEVKKPGASVKVSCKASGYTFTDFIIAWVKQAPGQGLEWIGEIYPGTGRTYYSEKFRGKATLTADTSTSTAYMELSSLRSEDTAVYFCARRTIYYDYDGDYWGQGTLVTVSS

SEQ ID NO: 22 - protein, 5 aa, Artificial Sequence
  TPP-7007 -  HCDR1
DFIIA

SEQ ID NO: 23 - protein, 17 aa, Artificial Sequence
  TPP-7007 -  HCDR2
EIYPGTGRTYYSEKFRG

SEQ ID NO: 24 - protein, 11 aa, Artificial Sequence
  TPP-7007 -  HCDR3
RTIYYDYDGDY

SEQ ID NO: 25 - protein, 112 aa, Artificial Sequence
  TPP-7007 -  VL
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAHNLELPWTFGQGTKVEIK

SEQ ID NO: 26 - protein, 16 aa, Artificial Sequence
  TPP-7007 -  LCDR1
RSSKSLLHSNGITYLY

SEQ ID NO: 27 - protein, 7 aa, Artificial Sequence
  TPP-7007 -  LCDR2
QMSNLAS

SEQ ID NO: 28 - protein, 9 aa, Artificial Sequence
  TPP-7007 -  LCDR3
AHNLELPWT

SEQ ID NO: 29 - protein, 449 aa, Artificial Sequence
  TPP-7007 -  Heavy Chain
QVQLVQSGAEVKKPGASVKVSCKASGYTFTDFIIAWVKQAPGQGLEWIGEIYPGTGRTYYSEKFRGKATLTADTSTSTAYMELSSLRSEDTAVYFCARRTIYYDYDGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 219 aa, Artificial Sequence
  TPP-7007 -  Light Chain
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAHNLELPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 120 aa, Artificial Sequence
  TPP-7053 -  VH
QVQLVQSGAEVKKPGASVKVSCKASGYTFTDFIIAWVKQAPGQGLEWKGEIYPGTGITYYSEKFRGKATKAALTSTSTAYMELSSLRSEDTAVYFCARRTFYYDYDGDYWGQGTLVTVSS

SEQ ID NO: 32 - protein, 5 aa, Artificial Sequence
  TPP-7053 -  HCDR1
DFIIA

SEQ ID NO: 33 - protein, 17 aa, Artificial Sequence
  TPP-7053 -  HCDR2
EIYPGTGITYYSEKFRG

SEQ ID NO: 34 - protein, 11 aa, Artificial Sequence
  TPP-7053 -  HCDR3
RTFYYDYDGDY

SEQ ID NO: 35 - protein, 112 aa, Artificial Sequence
  TPP-7053 -  VL
DIVMTQSPLSLPVTPGEPASISCRSSKSLLHSNGITYLYWYLQKPGQSYQLLIYQMSNLALGVPDRFSGSGSGTDFTLKISRVEAEDVGVYYCAHNLELPWTFGQGTKVEIK

SEQ ID NO: 36 - protein, 16 aa, Artificial Sequence
  TPP-7053 -  LCDR1
RSSKSLLHSNGITYLY

SEQ ID NO: 37 - protein, 7 aa, Artificial Sequence
  TPP-7053 -  LCDR2
QMSNLAL

SEQ ID NO: 38 - protein, 9 aa, Artificial Sequence
  TPP-7053 -  LCDR3
AHNLELPWT

SEQ ID NO: 39 - protein, 449 aa, Artificial Sequence
  TPP-7053 -  Heavy Chain
QVQLVQSGAEVKKPGASVKVSCKASGYTFTDFIIAWVKQAPGQGLEWKGEIYPGTGITYYSEKFRGKATKAALTSTSTAYMELSSLRSEDTAVYFCARRTFYYDYDGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 219 aa, Artificial Sequence
  TPP-7053 -  Light Chain
DIVMTQSPLSLPVTPGEPASISCRSSKSLLHSNGITYLYWYLQKPGQSYQLLIYQMSNLALGVPDRFSGSGSGTDFTLKISRVEAEDVGVYYCAHNLELPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 120 aa, Artificial Sequence; identical to SEQ ID NO: 11
  TPP-7065 -  VH

SEQ ID NO: 42 - protein, 5 aa, Artificial Sequence
  TPP-7065 -  HCDR1
DFIIA

SEQ ID NO: 43 - protein, 17 aa, Artificial Sequence
  TPP-7065 -  HCDR2
EIYPGTGRTYYSEKFRG

SEQ ID NO: 44 - protein, 11 aa, Artificial Sequence
  TPP-7065 -  HCDR3
RTIYYDYDGDY

SEQ ID NO: 45 - protein, 112 aa, Artificial Sequence; identical to SEQ ID NO: 15
  TPP-7065 -  VL

SEQ ID NO: 46 - protein, 16 aa, Artificial Sequence
  TPP-7065 -  LCDR1
RSSKSLLHSNGITYLY

SEQ ID NO: 47 - protein, 7 aa, Artificial Sequence
  TPP-7065 -  LCDR2
QMSNLAS

SEQ ID NO: 48 - protein, 9 aa, Artificial Sequence
  TPP-7065 -  LCDR3
AHNLELPWT

SEQ ID NO: 49 - protein, 450 aa, Artificial Sequence
  TPP-7065 -  Heavy Chain
QVQLQQSGPELVKPGASVKMSCKASGYTFTDFIIAWVKQRTGQGLEWIGEIYPGTGRTYYSEKFRGKATLTADKSSNTAYMQLSSLTSVDSAVYFCARRTIYYDYDGDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 50 - protein, 219 aa, Artificial Sequence
  TPP-7065 -  Light Chain
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAHNLELPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 51 - protein, 120 aa, Artificial Sequence; identical to SEQ ID NO: 1
  TPP-7073 -  VH

SEQ ID NO: 52 - protein, 5 aa, Artificial Sequence
  TPP-7073 -  HCDR1
DFIIA

SEQ ID NO: 53 - protein, 17 aa, Artificial Sequence
  TPP-7073 -  HCDR2
EIYPGTGRTYYSEKFRG

SEQ ID NO: 54 - protein, 11 aa, Artificial Sequence
  TPP-7073 -  HCDR3
RTIYYDYDGDY

SEQ ID NO: 55 - protein, 112 aa, Artificial Sequence
  TPP-7073 -  VL
DIVMTQSPLSLPVTPGEPASISCRSSKSLLHSSGITYLYWYLQKPGQSPQLLIYQMSNLASGVPDRFSSSGSGTDFTLKISRVEAEDVGVYYCAHNLELPWTFGGGTKVEIK

SEQ ID NO: 56 - protein, 16 aa, Artificial Sequence
  TPP-7073 -  LCDR1
RSSKSLLHSSGITYLY

SEQ ID NO: 57 - protein, 7 aa, Artificial Sequence
  TPP-7073 -  LCDR2
QMSNLAS

SEQ ID NO: 58 - protein, 9 aa, Artificial Sequence
  TPP-7073 -  LCDR3
AHNLELPWT

SEQ ID NO: 59 - protein, 449 aa, Artificial Sequence; identical to SEQ ID NO: 9
  TPP-7073 -  Heavy Chain

SEQ ID NO: 60 - protein, 219 aa, Artificial Sequence
  TPP-7073 -  Light Chain
DIVMTQSPLSLPVTPGEPASISCRSSKSLLHSSGITYLYWYLQKPGQSPQLLIYQMSNLASGVPDRFSSSGSGTDFTLKISRVEAEDVGVYYCAHNLELPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 120 aa, Artificial Sequence; identical to SEQ ID NO: 11
  TPP-7074 -  VH

SEQ ID NO: 62 - protein, 5 aa, Artificial Sequence
  TPP-7074 -  HCDR1
DFIIA

SEQ ID NO: 63 - protein, 17 aa, Artificial Sequence
  TPP-7074 -  HCDR2
EIYPGTGRTYYSEKFRG

SEQ ID NO: 64 - protein, 11 aa, Artificial Sequence
  TPP-7074 -  HCDR3
RTIYYDYDGDY

SEQ ID NO: 65 - protein, 112 aa, Artificial Sequence
  TPP-7074 -  VL
DIVMTQAAFSNPVTLGTSASISCRSSKSLLHSSGITYLYWYLQKPGQSPQLLIYQMSNLASGVPDRFSSSGSGTDFTLRISRVEAEDVGVYYCAHNLELPWTFGGGTKLELK

SEQ ID NO: 66 - protein, 16 aa, Artificial Sequence
  TPP-7074 -  LCDR1
RSSKSLLHSSGITYLY

SEQ ID NO: 67 - protein, 7 aa, Artificial Sequence
  TPP-7074 -  LCDR2
QMSNLAS

SEQ ID NO: 68 - protein, 9 aa, Artificial Sequence
  TPP-7074 -  LCDR3
AHNLELPWT

SEQ ID NO: 69 - protein, 449 aa, Artificial Sequence; identical to SEQ ID NO: 19
  TPP-7074 -  Heavy Chain

SEQ ID NO: 70 - protein, 219 aa, Artificial Sequence
  TPP-7074 -  Light Chain
DIVMTQAAFSNPVTLGTSASISCRSSKSLLHSSGITYLYWYLQKPGQSPQLLIYQMSNLASGVPDRFSSSGSGTDFTLRISRVEAEDVGVYYCAHNLELPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 120 aa, Artificial Sequence; identical to SEQ ID NO: 21
  TPP-7075 -  VH

SEQ ID NO: 72 - protein, 5 aa, Artificial Sequence
  TPP-7075 -  HCDR1
DFIIA

SEQ ID NO: 73 - protein, 17 aa, Artificial Sequence
  TPP-7075 -  HCDR2
EIYPGTGRTYYSEKFRG

SEQ ID NO: 74 - protein, 11 aa, Artificial Sequence
  TPP-7075 -  HCDR3
RTIYYDYDGDY

SEQ ID NO: 75 - protein, 112 aa, Artificial Sequence
  TPP-7075 -  VL
DIVMTQSPLSLPVTPGEPASISCRSSKSLLHSSGITYLYWYLQKPGQSPQLLIYQMSNLASGVPDRFSGSGSGTDFTLKISRVEAEDVGVYYCAHNLELPWTFGQGTKVEIK

SEQ ID NO: 76 - protein, 16 aa, Artificial Sequence
  TPP-7075 -  LCDR1
RSSKSLLHSSGITYLY

SEQ ID NO: 77 - protein, 7 aa, Artificial Sequence
  TPP-7075 -  LCDR2
QMSNLAS

SEQ ID NO: 78 - protein, 9 aa, Artificial Sequence
  TPP-7075 -  LCDR3
AHNLELPWT

SEQ ID NO: 79 - protein, 449 aa, Artificial Sequence; identical to SEQ ID NO: 29
  TPP-7075 -  Heavy Chain

SEQ ID NO: 80 - protein, 219 aa, Artificial Sequence
  TPP-7075 -  Light Chain
DIVMTQSPLSLPVTPGEPASISCRSSKSLLHSSGITYLYWYLQKPGQSPQLLIYQMSNLASGVPDRFSGSGSGTDFTLKISRVEAEDVGVYYCAHNLELPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 120 aa, Artificial Sequence; identical to SEQ ID NO: 31
  TPP-7076 -  VH

SEQ ID NO: 82 - protein, 5 aa, Artificial Sequence
  TPP-7076 -  HCDR1
DFIIA

SEQ ID NO: 83 - protein, 17 aa, Artificial Sequence
  TPP-7076 -  HCDR2
EIYPGTGITYYSEKFRG

SEQ ID NO: 84 - protein, 11 aa, Artificial Sequence
  TPP-7076 -  HCDR3
RTFYYDYDGDY

SEQ ID NO: 85 - protein, 112 aa, Artificial Sequence
  TPP-7076 -  VL
DIVMTQSPLSLPVTPGEPASISCRSSKSLLHSSGITYLYWYLQKPGQSYQLLIYQMSNLALGVPDRFSGSGSGTDFTLKISRVEAEDVGVYYCAHNLELPWTFGQGTKVEIK

SEQ ID NO: 86 - protein, 16 aa, Artificial Sequence
  TPP-7076 -  LCDR1
RSSKSLLHSSGITYLY

SEQ ID NO: 87 - protein, 7 aa, Artificial Sequence
  TPP-7076 -  LCDR2
QMSNLAL

SEQ ID NO: 88 - protein, 9 aa, Artificial Sequence
  TPP-7076 -  LCDR3
AHNLELPWT

SEQ ID NO: 89 - protein, 449 aa, Artificial Sequence; identical to SEQ ID NO: 39
  TPP-7076 -  Heavy Chain

SEQ ID NO: 90 - protein, 219 aa, Artificial Sequence
  TPP-7076 -  Light Chain
DIVMTQSPLSLPVTPGEPASISCRSSKSLLHSSGITYLYWYLQKPGQSYQLLIYQMSNLALGVPDRFSGSGSGTDFTLKISRVEAEDVGVYYCAHNLELPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 120 aa, Artificial Sequence; identical to SEQ ID NO: 11
  TPP-7077 -  VH

SEQ ID NO: 92 - protein, 5 aa, Artificial Sequence
  TPP-7077 -  HCDR1
DFIIA

SEQ ID NO: 93 - protein, 17 aa, Artificial Sequence
  TPP-7077 -  HCDR2
EIYPGTGRTYYSEKFRG

SEQ ID NO: 94 - protein, 11 aa, Artificial Sequence
  TPP-7077 -  HCDR3
RTIYYDYDGDY

SEQ ID NO: 95 - protein, 112 aa, Artificial Sequence; identical to SEQ ID NO: 65
  TPP-7077 -  VL

SEQ ID NO: 96 - protein, 16 aa, Artificial Sequence
  TPP-7077 -  LCDR1
RSSKSLLHSSGITYLY

SEQ ID NO: 97 - protein, 7 aa, Artificial Sequence
  TPP-7077 -  LCDR2
QMSNLAS

SEQ ID NO: 98 - protein, 9 aa, Artificial Sequence
  TPP-7077 -  LCDR3
AHNLELPWT

SEQ ID NO: 99 - protein, 450 aa, Artificial Sequence; identical to SEQ ID NO: 49
  TPP-7077 -  Heavy Chain

SEQ ID NO: 100 - protein, 219 aa, Artificial Sequence
  TPP-7077 -  Light Chain
DIVMTQAAFSNPVTLGTSASISCRSSKSLLHSSGITYLYWYLQKPGQSPQLLIYQMSNLASGVPDRFSSSGSGTDFTLRISRVEAEDVGVYYCAHNLELPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 101 - protein, 129 aa, Homo sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 102 - DNA, 959 bp, Homo sapiens
atggctcggggctcgctgcgccggttgctgcggctcctcgtgctggggctctggctggcgttgctgcgctccgtggccggggagcaagcgccaggcaccgccccctgctcccgcggcagctcctggagcgcggacctggacaagtgcatggactgcgcgtcttgcagggcgcgaccgcacagcgacttctgcctgggctgcgctgcagcacctcctgcccccttccggctgctttggcccatccttgggggcgctctgagcctgaccttcgtgctggggctgctttctggctttttggtctggagacgatgccgcaggagagagaagttcaccacccccatagaggagaccggcggagagggctgcccagctgtggcgctgatccagtgacaatgtgccccctgccagccggggctcgcccactcatcattcattcatccattctagagccagtctctgcctcccagacgcggcgggagccaagctcctccaaccacaaggggggtggggggcggtgaatcacctctgaggcctgggcccagggttcaggggaaccttccaaggtgtctggttgccctgcctctggctccagaacagaaagggagcctcacgctggctcacacaaaacagctgacactgactaaggaactgcagcatttgcacaggggaggggggtgccctccttccttaggacctgggggccaggctgacttggggggcagacttgacactaggccccactcactcagatgtcctgaaattccaccacgggggtcaccctggggggttagggacctatttttaacactaggggctggcccactaggagggctggccctaagatacagacccccccaactccccaaagcggggaggagatatttattttggggagagtttggaggggagggagaatttattaataaaagaatctttaactttaaaaaaaaaaaaaaaaa